(19) United States
(12) Patent Application Publication
(10) Pub. No.: US 2025/0336533 A1
(43) Pub. Date: Oct. 30, 2025

(54) Methods and Systems for Evaluation of Lupus Based on Ancestry-Associated Molecular Pathways

(71) Applicant: AMPEL BioSolutions, LLC, Charlottesville, VA (US)

(72) Inventors: Katherine A. OWEN, Charlottesville, VA (US); Amrie C. GRAMMER, Charlottesville, VA (US); Peter E. LIPSKY, Charlottesville, VA (US)

(21) Appl. No.: 19/199,682

(22) Filed: May 6, 2025

Related U.S. Application Data
(63) Continuation of application No. PCT/US2023/032947, filed on Sep. 15, 2023.
(60) Provisional application No. 63/424,420, filed on Nov. 10, 2022.

(51) Int. Cl. G16H 50/20; G16B 20/10; G16B 20/20; G16B 40/20; G16H 10/40

(52) U.S. Cl. CPC G16H 50/20; G16B 20/10; G16B 20/20; G16B 40/20; G16H 10/40

(57) ABSTRACT

Methods and systems for diagnosis and treatment of lupus in a patient is disclosed. The method can include analyzing a data set comprising or derived from gene expression measurements of at least 2 genes selected from the genes listed in each of one or more Tables selected from Tables: 1 to 11 to determine a set of genes enriched in a biological sample obtained or derived from the patient, and diagnosing lupus in the patient based on enrichment of the set of genes, wherein the gene expression measurements are obtained from the biological sample.

### CROSS-REFERENCE

This application is a continuation of PCT/US2023/032947, filed on Sep. 15, 2023, which claims the benefit of U.S. Provisional Patent Application No. 63/424,420, filed on Nov. 10, 2022, the contents of which are incorporated herein by reference in their entirety.

### BACKGROUND

Lupus, including Systemic Lupus Erythematosus (SLE), is heterogeneous in nature, and has variable causation, course and responsiveness to therapy. Genetics plays a role in both SLE susceptibility and severity, however molecular pathways contributing to SLE disease pathogenesis remains poorly understood. Individuals of East Asian ancestry (AsA) have a greater prevalence of renal involvement, infections and cardiovascular complications compared to individuals of European ancestry (EA). In particular, lupus nephritis and end stage renal disease (LN/ESRD) are severe complications of SLE that are more prevalent in patients of AsA ancestry than patients of EA ancestry. Whereas some of this variation may be accounted for by confounding environmental and/or socioeconomic factors, it is unclear why AsA ancestry remains associated with clinical severity and sub-phenotypes in SLE. There is a need for understanding molecular pathways involved in the pathogenesis of these conditions to allow identification and optimization of therapies.

### SUMMARY

Methods of the current disclosure can determine molecular pathways involved in development of lupus in a patient. Based on enrichment of genes associated with specific molecular pathways, methods of the current invention can diagnose lupus in a patient, and can provide optimized therapy to the patient.

The following Aspects are disclosed.

Aspect 1 is directed to a method for diagnosis of lupus in a patient, the method comprising:
a) analyzing a data set comprising or derived from gene expression measurements of at least 2 genes selected from the genes listed in each of one or more Tables selected from Tables: 1 to 11 to determine one or more sets of genes enriched in a biological sample obtained or derived from the patient; and
b) diagnosing lupus in the patient based on enrichment of the one or more sets of genes,
   wherein the gene expression measurements are obtained from the biological sample.

Aspect 2 is directed to the method of aspect 1, wherein the data set comprises or is derived from gene expression measurements of effective number of genes selected from the genes listed in each of the one or more Tables selected from Tables: 1 to 11.

Aspect 3 is directed to the method of aspect 1, wherein the data set comprises or is derived from gene expression measurements of all genes listed in each of the one or more Tables selected from Tables: 1 to 11.

Aspect 4 is directed to the method of any one of aspects 1 to 3, wherein Tables: 1 to 11 are selected.

Aspect 5 is directed to the method of any one of aspects 1 to 4, wherein the data set is derived from the gene expression measurements using GSVA, gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.

Aspect 6 is directed to the method of any one of aspects 1 to 5, wherein the data set is derived from the gene expression measurements using GSVA.

Aspect 7 is directed to the method of aspect 6, wherein the data set comprises one or more GSVA scores of the patient, each GSVA score generated based on one of the one or more selected Tables, wherein for each selected Table, the genes selected from the selected Table forms the input gene set for generating the GSVA score based on the selected Table, using GSVA.

Aspect 8 is directed to the method of any one of aspects 1 to 7, further comprising administering a treatment to the patient based on the enrichment of the set of genes.

Aspect 9 is directed to the method of aspect 8, wherein the treatment is configured to treat lupus.

Aspect 10 is directed to the method aspect 8, wherein the treatment is configured to reduce severity of lupus.

Aspect 11 is directed to the method aspect 8, wherein the treatment is configured to reduce risk of having lupus.

Aspect 12 is directed to the method of any one of aspects 8 to 11, wherein: the one or more sets of genes comprise a set of genes selected from Table 1, and the treatment targets a JAK signaling pathway; the one or more sets of genes comprise a set of genes selected from Table 2, and the treatment targets an oxidative phosphorylation pathway; the one or more sets of genes comprise a set of genes selected from Table 3, and the treatment targets a sirtuin signaling pathway; the one or more sets of genes comprise a set of genes selected from Table 4, and the treatment targets a mitochondrial dysfunction pathway; the one or more sets of genes comprise a set of genes selected from Table 5, and the treatment targets a glycolysis pathway; the one or more sets of genes comprise a set of genes selected from Table 6, and the treatment targets a reactive oxygen species (ROS) protection pathway; the one or more sets of genes comprise a set of genes selected from Table 7, and the treatment targets an MTOR signaling pathway; the one or more sets of genes comprise a set of genes selected from Table 8, and the treatment targets a JAK signaling pathway; the one or more sets of genes comprise a set of genes selected from Table 9, and the treatment targets a microRNA processing pathway; the one or more sets of genes comprise a set of genes selected from Table 10, and the treatment targets a mitochondrial dysfunction pathway; the one or more sets of genes comprise a set of genes selected from Table 11, and the treatment targets a TNF signaling pathway; or any combination thereof.

Aspect 13 is directed to the method of aspect 12, wherein the treatment targeting the JAK signaling pathway comprises baricitinib, carfilzomib, curcumol, decernotinib, delgocitinib, ruxolitinib, solicitinib, tofacitinib, upadacitinib, bortezomib, densosumab, filgotinib, idelalisib, KZR-616, peficitinib, or any combination thereof; the treatment targeting the oxidative phosphorylation pathway comprises metformin, phenformin, BAY84-2243, CAI, ME344, fenofibrate, lonidamine, arsenic trioxide, atovaquone, hydrocortisone, a-TOS, thapsigargin, or any combination thereof; the treatment targeting the sirtuin signaling pathway comprises resveratrol, and/or cyclosporin A; the treatment targeting the mitochondrial dysfunction pathway comprises resveratrol, N-acetyl L-cysteine, SKQ1, ubiquinone, mitoVitE, mitoTEMPO, vitamin E, vitamin C, or any combination thereof; the treatment targeting the glycolysis pathway comprises Cylcosporin A; the treatment targeting the reactive oxygen species (ROS) protection pathway comprises resveratrol, N-acetyl L-cysteine, SKQ1, ubiquinone, mito VitE, mitoTEMPO, vitamin E, vitamin C, ALT-2074, Ebselen, GC4419, or any combination thereof; the treatment targeting the MTOR signaling pathway comprises sirolimus, everolimus, temsirolimus, or any combination thereof; the treatment targeting microRNA processing pathway comprises cyclosporin A, and/or thapsigargin; treatment targeting the TNF signaling pathway comprises adalimumab, AMG-811, baricitinib, BMS-986165, certolizumab, dacomitinib, etanercept, filgotinib, iguratimod, infliximab, ruxolitinib, solicitinib, tabalumab, trofinetide, upadacitinib, or any combination thereof.

Aspect 14 is directed to the method of any one of aspects 1 to 13, wherein the biological sample comprises a blood sample, isolated peripheral blood mononuclear cells (PBMCs), a tissue biopsy sample, or any derivative thereof.

Aspect 15 is directed to the method of any one of aspects 1 to 13, wherein the biological sample comprises a blood sample, isolated peripheral blood mononuclear cells (PBMCs), or any derivative thereof.

Aspect 16 is directed to the method of any one of aspects 1 to 15, wherein the patient has lupus.

Aspect 17 is directed to the method of any one of aspects 1 to 15, wherein the patient is at elevated risk of having lupus.

Aspect 18 is directed to the method of any one of aspects 1 to 15, wherein the patient is suspected of having lupus.

Aspect 19 is directed to the method of any one of aspects 1 to 15, wherein the patient is asymptomatic for lupus.

Aspect 20 is directed to the method of any one of aspects 1 to 19, wherein the patient is of Asian ancestry and/or European ancestry.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent application file contains at least one drawing executed in color. Copies of this patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-F: Key pathways determined by EA and AsA-associated genes. Venn diagrams depicting the ancestral overlap of all SLE-associated Immunochip SNPs (FIG. 1A) and the overlap between all EA- and AsA-SNP predicted genes (FIG. 1B). Cluster metastructures for EA (FIG. 1C), AsA (FIG. 1D) and the shared gene cohort (FIG. 1E) were generated based on protein-protein interaction (PPI) networks, clustered using MCODE and visualized in Cytoscape. Cluster size indicates the number of genes per cluster, edge weight indicates the number of inter-cluster connections and color indicates the number of intra-cluster connections. Enrichment for each cluster was determined by BIG-C and IPA; clusters were then grouped and categorized according to overall function (immune, tissue repair, metabolic, motility or general). Grey boxes indicate categories lacking relevant clusters. FIG. 1F. Venn diagram showing the number of overlapping pathways motivated by EA or AsA predicted genes. Representative pathways are listed. Node size, node color, edge weight, and edge color scale for FIGS. 1C, 1D and 1E is shown in FIG. 1D.

FIGS. 2A-C: AsA Immunochip-based pathways are supported by summary GWAS from AsA SLE patients. Using SNP-predicted genes from the AsA GWAS validation SNP-set (FIG. 2A) or an equivalently sized cohort of random genes (FIG. 2B) metastructures were generated based on PPI networks, clustered using MCODE and visualized in Cytoscape. Cluster size indicates the number of genes per cluster, edge weight indicates the number of inter-cluster connections and color indicates the number of intra-cluster connections. Enrichment for each cluster was determined by BIG-C and IPA; clusters were then grouped and categorized according to overall function Grey boxes indicate categories lacking relevant clusters. (FIG. 2C) Quantitation of AsA GWAS (black bars/upper bar in each category) and random (red bars/lower bar in each category) genes falling into each BIG-C category and grouped by overall functionality. Node size, node color, edge weight, and edge color scale for FIGS. 2A, and 2B is shown in FIG. 2B.

FIGS. 3A-F: SNP-associated pathways inform gene signatures for GSVA analysis in patient PBMC datasets. FIGS. 3A-B. GSVA enrichment scores for metabolic processes were generated for PBMCs in EA and AsA SLE patients and healthy controls from FDAPBMC1 (EA-only patients and controls) and GSE81622 (AsA-only patients and controls). Asterisks (*) indicate a p-value <0.05 using Welch's t-test comparing SLE to control. (FIGS. 3C-D) Using gene expression from purified CD14+ monocytes (GSE164457), linear regression was used to examine the relationship between cellular processes and SLEDAI and anti-dsDNA titers in active EA and AsA patients (SLEDAI≥6) (FIGS. 3E-F).

FIG. 4: A schematic of non-limiting pathways involved in development of lupus in patients of Asian and European ancestry, and non-limiting examples of treatments associated with the pathways.

FIGS. 5A-E: Mapping the functional genes associated with SLE-Immunochip SNPs. (FIG. 5A) Venn diagram depicting the ancestral overlap of all SLE-associated Immunochip SNPs. (FIG. 5B) Distribution of genomic functional categories for all EA and AsA non-HLA associated SLE SNPs. Genomic category comparisons between ancestral groups were performed using a 2-proportion z test. P values were 2-tailed, and asterisks indicate a significance threshold of p<0.05. For, FIG. 5B left bar diagram, the coding, Non-coding, regulatory and ncRNA SNPs are represented from bottom to top. For, FIG. 5B right bar diagram, the 3′UTR, 5′UTR, synonomous and Mis/nonsense coding regions SNPs are represented from bottom to top. (FIG. 5C) Functional SNP-associated genes are derived from 4 sources, including eQTL analysis (E-Genes), regulatory regions (T-Genes), coding regions (C-Genes) and proximal gene-SNP annotation (P-Genes). (FIGS. 5D and E) Venn diagrams showing the overlap of all EA (FIG. 5D) and AsA (FIG. 5E) associated E-, T-, C- and P-Genes.

FIG. 6: Immunochip SNPs exhibiting eQTL effects are more frequent in Asian Ancestry. EA and AsA Immunochip SNPs designated as eQTL via the GTEx and Blood eQTL browser databases were distributed into their genomic functional categories. Numbers above each bar indicate the total number of SNPs in each category. Bottom (dark shading), eQTL; Top (light shading), non-eQTL.

FIGS. 7A-E: Functional characterization of SNP-associated genes. (FIG. 7A) Venn diagram depicting the overlap between all EA- and AsA-SNP associated genes. (FIGS. 7B, 7C) Bubble plots depict ancestry-dependent and independent SNP-associated genes analyzed to determine enrichment using functional definitions from the BIG-C (Biologically Informed Gene Clustering) annotation library and I-Scope for hematopoietic cell enrichment. Enrichment was defined as any category with an odds ratio (OR)>1 and a −log (p-value)>1.33. (FIG. 7D) Heatmap (generated by GraphPad Prism 8.3; www.graphpad.com) visualization of the top five significant IPA canonical pathways and (FIG. 7E) bubble plot showing gene ontogeny (GO) terms for each gene list organized by ancestry. Top pathways with OR>1 and −log (p-value)>1.33 are listed.

FIGS. 8A-E: Functional characterization of SNP-associated E-T-C-Genes. (FIG. 8A) Venn diagram depicting the overlap between SNP associated E-T-C EA- and AsA genes (excluding P-Genes). (FIG. 8B-8C) Bubble plots depict E-T-C ancestry-dependent and independent SNP-associated genes analyzed to determine enrichment using functional definitions from the BIG-C (Biologically Informed Gene Clustering) annotation library and I-Scope for hematopoietic cell enrichment. Enrichment was defined as any category with an odds ratio (OR)>1 and a −log (p-value)>1.33. EA and AsA P-Genes were analyzed separately. (FIG. 8D) Heatmap visualization of the top three significant IPA canonical pathways and (FIG. 8E) bubble plot showing gene ontogeny (GO) terms for each gene list organized by ancestry. Top pathways with OR>1 and −log (p-value)>1.33 are listed.

FIGS. 9A-D: Key pathways determined by EA and AsA-associated genes. Cluster metastructures for EA (FIG. 9A), AsA (FIG. 9B) and the shared gene cohort (FIG. 9C) were generated based on PPI networks, clustered using MCODE and visualized in Cytoscape. Cluster size indicates the number of genes per cluster, edge weight indicates the number of inter-cluster connections and color indicates the number of intra-cluster connections. Enrichment for each cluster was determined by BIG-C and IPA; clusters were then grouped and categorized according to overall function (immune, tissue repair, metabolic, motility or general), Grey boxes indicate categories lacking relevant clusters. (FIG. 9D) Venn diagram showing the number of overlapping pathways motivated by EA or AsA predicted genes. Representative pathways are listed.

FIGS. 10A-B: Key pathways determined by all EA and AsA-associated genes. Cluster metastructures using the full cohort of EA (FIG. 10A) and AsA (FIG. 10B) genes were generated based on PPI networks, clustered using MCODE and visualized in Cytoscape. Cluster size indicates the number of genes per cluster, edge weight indicates the number of inter-cluster connections and color indicates the number of intra-cluster connections. Enrichment for each cluster was determined by BIG-C and IPA; clusters were then grouped and categorized according to overall function (immune, tissue repair, metabolic, motility or general). Grey boxes indicate categories lacking relevant clusters.

FIGS. 11A-C: Distribution of genomic functional categories for GWAS validation cohort SNPs. (FIG. 11A) The genomic functional categories for all GWAS validation SLE SNPs was determined. Coding region SNPs were further broken down based on their location. Numbers above each bar indicate the total number of SNPs in each category. In FIG. 11A left bar diagram, the coding, Non-coding, regulatory and ncRNA SNPs are represented from bottom to top. In FIG. 11A right bar diagram, the 3′UTR, 5′UTR, synonomous and Mis/nonsense coding regions SNPs are represented from bottom to top. (FIGS. 11B-1C) Venn diagrams depicting the ancestral overlap of all Immunochip and GWAS SNPs and predicted genes.

FIGS. 12A-D: AsA Immunochip-based pathways are supported by summary GWAS from AsA SLE patients. Using SNP-predicted genes from the AsA GWAS validation SNP-set (FIG. 12A) or an equivalently sized cohort of random genes (FIG. 12B) metastructures were generated based on PPI networks, clustered using MCODE and visualized in Cytoscape. Cluster size indicates the number of genes per cluster, edge weight indicates the number of inter-cluster connections and color indicates the number of intra-cluster connections. Enrichment for each cluster was determined by BIG-C and IPA; clusters were then grouped and categorized according to overall function (immune, tissue repair, metabolic, motility or general). Grey boxes indicate categories lacking relevant clusters. (FIG. 12C) Quantitation of cluster size, intra-cluster connections and inter-cluster connections network is displayed. Error bars represent the 95% confidence interval; asterisks (***) indicate a p-value <0.001 using Welch's t-test. (FIG. 12D) Quantitation of AsA GWAS (black bars/upper bar in each category) and random (red/lower bar in each category) genes falling into each BIG-C category and grouped by overall functionality.

FIGS. 13A-B: Key pathways determined by AsA differentially expressed genes. (FIG. 13A) Differentially expressed AsA genes were examined for functional and cellular enrichment using BIG-C and I-Scope, respectively. Bubble plot depicts significantly enriched categories (−log (pvalue)>1.33; OR>1). (FIG. 13B) Top GO Biological and IPA canonical pathways (−log (p-value)>1.33) for all AsA DEGs.

FIGS. 14A-B: Key overlapping pathways determined by SNP-predicted and differentially expressed genes. (FIG. 14A) Venn diagram depicting the numerical overlap between AsA SNPpredicted genes (SPGs), EA SPGs and AsA DEGs. (FIG. 14B) Top GO Biological pathways determined by each group of overlapping genes (−log(p-value)>1.33).

FIG. 15A-B: Asian-associated pathways are validated with gene expression data from AsA SLE patients. (FIG. 15A) Using differentially expressed (DE) genes from AsA whole blood samples (E-MTAB-11191), metastructures were generated based on PPI networks, clustered using MCODE and visualized in Cytoscape. Cluster size indicates the number of genes per cluster, edge weight indicates the number of inter-cluster connections and color indicates the number of intra-cluster connections. Enrichment for each cluster was determined by BIG-C and IPA; clusters were then grouped and categorized according to overall function (immune, tissue repair, metabolic, motility [Continued from previous page] or genera cell funtion1. (FIG. 15B) Bar graph showing the precent of associated (EA/AsA immunochip and AsA GWAS), differentially expressed and random genes falling into each overall functional category.

FIGS. 16A-H: SNP-associated pathways inform gene signatures for GSVA analysis in patient PBMC datasets. GSVA enrichment scores were generated for PBMCs in EA and AsA SLE patients and healthy controls from FDAPBMC1 (EA-only patients and controls) and GSE81622 (AsA-only patients and controls). GSVA scores for type I and type II interferon-based gene signatures (FIGS. 16A, 16B), metabolic gene signatures (FIGS. 16C, 16D), cellular processes (FIGS. 16E, 16F) and individual cell type signatures (FIGS. 16G, 16H) are shown. Asterisks (*) indicate a p-value <0.05 using Welch's t-test comparing SLE to control.

FIGS. 17A-E: Linear regression to examine the relationship between cell types, biological processes and inflammatory cytokines. (FIG. 17A) Linear regression analysis showing the relationship between GSVA scores for glycolysis, oxidative phosphorylation or oxidative stress and individual cell types (pDCs, monocyte/myeloid, B cells, T cells and NK cells) for FDAPBMC1 (EA, upper panels) and GSE81622 (AsA, lower panels). FIG. 17A top left figure, at the highest shown GSVA score, the lines positioned from top to bottom are NK cell, pDC, T cell, Mono/mye, B cell. FIG. 17A top middle figure, at the highest shown GSVA score, the lines positioned from top to bottom are T cell, NK cell, B cell, pDC, Mono/mye. FIG. 17A top right figure, at the highest shown GSV A score, the lines positioned from top to bottom are NK cell, Mono/mye, pDC, B cell, T cell. FIG. 17A bottom left figure, at the highest shown GSVA score, the lines positioned from top to bottom are Mono/mye, B cell, pDC, T cell, NK cell. FIG. 17A bottom middle figure, at the highest shown GSVA score, the lines positioned from top to bottom are Mono/mye, B cell, T cell, NK cell, pDC. FIG. 17A bottom right figure, at the highest shown GSVA score, the lines positioned from top to bottom are Mono/mye, NK cell, B cell, T cell, pDC. In (FIG. 17B), GSVA enrichment scores for the indicated cellular processes were generated for purified CD14+ monocytes from EA and AsA SLE patients (GSE164457). Using GSE164457, linear regression was used to examine the relationship between cellular processes and SLEDAI (FIG. 17C), anti-dsDNA titers in active patients (SLEDAI≥6) (FIG. 17D) and GSVA scores for IFNA2 (FIG. 17E). FIG. 17E top figure, at the highest shown GSVA score, the lines positioned from top to bottom are DNA/RNA (0.62*), TLR (0.17*), Mito. dys. (0.01). FIG. 17E bottom figure, at the highest shown GSVA score, the lines positioned from top to bottom are DNA/RNA (0.73*), TLR (0.43*), Mito. dys. (0.07*). Categories with linear regression p values <0.05 are in bold; R² predictive values are listed after the GSVA enrichment category. * Asterisks indicate significant relationship between functional categories. N.s., not significant.

FIGS. 18A-C: Complement depletion is associated with anti-dsDNA titers and SLEDAI in AsA SLE patients. (FIG. 18A) Comparison of complement C3 levels in EA and AsA SLE patients (GSE164457). Asterisks (*) indicate a p-value <0.05 using Welch's t-test. (FIG. 18B-18C) Linear regression demonstrating the relationship between complement C3 levels and anti-dsDNA titers and disease activity as measured by SLE disease activity index (SLEDAI). R² predictive values and p-values are listed. N.s., not significant.

### DETAILED DESCRIPTION

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Many complex and multi-systematic diseases and conditions currently pose major diagnostic and therapeutic challenges. Despite the wealth of records from, for example, genetic, epigenetic, and gene expression data that has emerged in the past few years, physicians often still rely on clinical evaluation and laboratory tests, including measurement of autoantibodies and complement levels.

Successful relation of records (e.g., gene expression records) to a specific disease phenotype activity has been attempted, including efforts to identify individual genes that predicted subsequent flares, and through the determination of a discrete group of differentially expressed (DE) genes that may be found in a particular record. Despite these advances, however, no such approach is available with sufficient predictive value to utilize in evaluation and treatment.

As such, there is a need for a predictive tool for evaluating patient at both the chemical and cellular levels to advance personalized treatment. Data analytical techniques such as machine learning enable proper correlation between genetic records and phenotypes.

The methods described herein provide the basis of personalized medicine. Integration of the methods herein with emerging high-throughput record sampling technologies may unlock the potential to develop a simple blood test to predict phenotypic activity. The disclosures herein may be generalized to predict other manifestations, such as organ involvement. A better understanding of the cellular processes that drive pathogenesis may eventually lead to customized therapeutic strategies based on records' unique patterns of cellular activation.

One aspect of the present disclosure is directed to a method for diagnosis of lupus in a patient. The method can include, analyzing a data set comprising or derived from gene expression measurements of at least 2 genes. The data set can be analyzed to determine a set of genes enriched in a biological sample obtained or derived from the patient. The method can diagnose whether the patient has lupus based on enrichment of the sets of genes. In some embodiments, the at least 2 genes are selected from the genes listed in each of one or more Tables selected from Tables: 1 to 11, 14, 15, 16, 17, 19, 20, 21 and 22. In some embodiments, the at least 2 genes are selected from the genes listed in each of one or more Tables selected from Tables: 1 to 11. In some embodiments, the at least 2 genes are selected from the genes listed in each of one or more Tables selected from Tables: 1 to 11, to determine the set of genes enriched in the biological sample obtained or derived from the patient. The method can include diagnosing lupus in the patient based on enrichment of the set of genes. As a non-limiting example, Tables 1, 2 and 3 can be selected from Tables: 1 to 11, wherein the dataset comprises or is derived from gene expression measurements of at least 2 genes selected from the genes listed in each of the selected Tables, i.e., the dataset comprises or is derived from gene expression measurements of at least 2 genes selected from the genes listed in Table 1, at least 2 genes selected from the genes listed in Table 2, and at least 2 genes selected from the genes listed in Table 3. In certain embodiments, the data set comprises or is derived from gene expression measurements of effective number of genes selected from the genes listed in each of the one or more Tables selected from Tables: 1 to 11, wherein a different or identical number of genes are selected from the genes listed in each selected table. In certain embodiments, the data set comprises or is derived from gene expression measurements of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all, or any range or value there between genes selected from the genes listed in each of the one or more Tables selected from Tables: 1 to 11, wherein a different or identical number of genes are selected from the genes listed in each selected table. In certain embodiments, the data set comprises or is derived from gene expression measurements of all genes listed in each of the one or more Tables selected from Tables: 1 to 11, as a non-limiting examples, Tables 1, and 2 can be selected from Tables: 1 to 11, wherein the dataset can comprise or be derived from gene expression measurements of all the genes listed in each of the selected Tables, i.e., the dataset can comprises or be derived from gene expression measurements of all genes listed in Table 1, and all genes listed in Table 2. In certain embodiments, the one or more Tables comprise 1 to 11 Tables, i.e., 1 to 11 Tables are selected from Tables: 1 to 11. In certain embodiments, the one or more Tables comprise 1 to 2, 1 to 3, 1 to 4, 1 to 5, 1 to 6, 1 to 7, 1 to 8, 1 to 9, 1 to 10, 1 to 11, 2 to 3, 2 to 4, 2 to 5, 2 to 6, 2 to 7, 2 to 8, 2 to 9, 2 to 10, 2 to 11, 3 to 4, 3 to 5, 3 to 6, 3 to 7, 3 to 8, 3 to 9, 3 to 10, 3 to 11, 4 to 5, 4 to 6, 4 to 7, 4 to 8, 4 to 9, 4 to 10, 4 to 11, 5 to 6, 5 to 7, 5 to 8, 5 to 9, 5 to 10, 5 to 11, 6 to 7, 6 to 8, 6 to 9, 6 to 10, 6 to 11, 7 to 8, 7 to 9, 7 to 10, 7 to 11, 8 to 9, 8 to 10, 8 to 11, 9 to 10, 9 to 11, or 10 to 11 Tables. In certain embodiments, the one or more Tables comprise 1, 2, 3, 4, 5, 6, 7, 8, 9, 10, or 11 Tables. In certain embodiments, the one or more Tables comprise at least 1, 2, 3, 4, 5, 6, 7, 8, 9, or 10 Tables. In certain embodiments, Tables: 1 to 11 are selected. In certain embodiments, Tables: 1 to 11 are selected, and for each selected Table all genes listed in the selected Table are selected.

In some embodiments, the at least 2 genes are selected from the genes listed in Table 14. In some embodiments, the at least 2 genes are selected from the genes listed in Table 15. In some embodiments, the at least 2 genes are selected from the genes listed in Table 16. In some embodiments, the at least 2 genes are selected from the genes listed in Table 17. In some embodiments, the at least 2 genes are selected from the genes listed in Table 18. In some embodiments, the at least 2 genes are selected from the genes listed in Table 19. In some embodiments, the at least 2 genes are selected from the genes listed in Table 20. In some embodiments, the at least 2 genes are selected from the genes listed in Table 21. In some embodiments, the at least 2 genes are selected from the genes listed in Table 22. In some embodiments, the at least 2 genes are selected from each of one or more gene clusters selected from the gene clusters (e.g., MCODE clusters) listed in Table 15. In some embodiments, the at least 2 genes are selected from each of one or more gene clusters selected from the gene clusters listed in Table 16. In some embodiments, the at least 2 genes are selected from each of one or more gene clusters selected from the gene clusters listed in Table 17. In some embodiments, the at least 2 genes are selected from each of one or more gene clusters selected from the gene clusters listed in Table 20. In some embodiments, the at least 2 genes are selected from each of one or more gene clusters selected from the gene clusters listed in Table 21. In some embodiments, the at least 2 genes are selected from each of one or more gene clusters selected from the gene clusters listed in Table 22. Each gene clusters listed in Tables 14, 15, 16, 17, 19, 20, 21 and 22, can be effective biomarkers for lupus. One or more gene clusters selected from Table 15, 16, 17, 20, 21 or 22, can include at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or all genes clusters listed in the respective Table. In certain embodiments, the data set comprises or is derived from gene expression measurements of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, all, or any range or value therebetween, genes selected from the genes listed in each of the one or more gene clusters selected from Table 15, 16, 17, 20, 21 or 22, wherein a different or identical number of genes are selected from the genes listed in each selected table. In certain embodiments, the data set comprises or is derived from gene expression measurements of effective number of genes selected from the genes listed in each of the one or more gene clusters selected from Table 15, wherein a different or identical number of genes are selected from the genes listed in each selected gene cluster. In certain embodiments, the data set comprises or is derived from gene expression measurements of effective number of genes selected from the genes listed in each of the one or more gene clusters selected from Table 16, wherein a different or identical number of genes are selected from the genes listed in each selected gene cluster. In certain embodiments, the data set comprises or is derived from gene expression measurements of effective number of genes selected from the genes listed in each of the one or more gene clusters selected from Table 17, wherein a different or identical number of genes are selected from the genes listed in each selected gene cluster. In certain embodiments, the data set comprises or is derived from gene expression measurements of effective number of genes selected from the genes listed in each of the one or more gene clusters selected from Table 20, wherein a different or identical number of genes are selected from the genes listed in each selected gene cluster. In certain embodiments, the data set comprises or is derived from gene expression measurements of effective number of genes selected from the genes listed in each of the one or more gene clusters selected from Table 21, wherein a different or identical number of genes are selected from the genes listed in each selected gene cluster. In certain embodiments, the data set comprises or is derived from gene expression measurements of effective number of genes selected from the genes listed in each of the one or more gene clusters selected from Table 22, wherein a different or identical number of genes are selected from the genes listed in each selected gene cluster. In certain embodiments, the data set comprises or is derived from gene expression measurements of all the genes listed in Table 14. In certain embodiments, the data set comprises or is derived from gene expression measurements of all the genes listed in each of the one or more gene clusters selected from Table 15. In certain embodiments, the data set comprises or is derived from gene expression measurements of all the genes listed in each of the one or more gene clusters selected from Table 16. In certain embodiments, the data set comprises or is derived from gene expression measurements of all the genes listed in each of the one or more gene clusters selected from Table 17. In certain embodiments, the data set comprises or is derived from gene expression measurements of all the genes listed in Table 19. In certain embodiments, the data set comprises or is derived from gene expression measurements of all the genes listed in each of the one or more gene clusters selected from Table 20. In certain embodiments, the data set comprises or is derived from gene expression measurements of all the genes listed in each of the one or more gene clusters selected from Table 21. In certain embodiments, the data set comprises or is derived from gene expression measurements of all the genes listed in each of the one or more gene clusters selected from Table 22. In some embodiments, the patient is of European ancestry, and the one or more clusters selected from Table 15 includes clusters listed in Table 15G. In some embodiments, the patient is of Asian ancestry, and the one or more clusters selected from Table 15 includes clusters listed in Table 15H.

The data set can be generated from the biological sample obtained or derived from the patient. For example, nucleic acid molecules of the patient in the biological sample can be assessed to obtain the data set. In certain embodiments, the gene expression measurements of the biological sample of the selected genes can be performed using any suitable method known to those of skill in the art including but not limited to DNA sequencing, RNA sequencing, microarray, RNA-Seq, qPCR, northern blotting, fluorescent in situ hybridization, serial analysis of gene expression, tiling arrays or any combination thereof, to obtain the data set. In certain embodiments, the gene expression measurements of the biological sample of the selected genes can be performed using RNA-Seq. In certain embodiments, the gene expression measurements of the biological sample of the selected genes can be performed using microarray. In certain embodiments, the data set can be derived from the gene expression measurements of the biological sample, wherein the gene expression measurements is analyzed using a suitable data analysis tool including but not limited to a BIG-C™ big data analysis tool, an I-Scope™ big data analysis tool, a T-Scope™ big data analysis tool, a CellScan big data analysis tool, an MS (Molecular Signature) Scoring™ analysis tool, gene set variation analysis (GSVA), Z-score, gene set enrichment analysis (GSEA), enrichment algorithm, multiscale embedded gene co-expression network analysis (MEGENA), weighted gene co-expression network analysis (WGCNA), differential expression analysis, log 2 expression analysis, or any combination thereof, to obtain the dataset. In certain embodiments, the gene expression measurements of the biological sample can be analyzed using GSVA, to obtain the data set. In certain embodiments, the method comprises obtaining and/or deriving the biological sample from the patient. In certain embodiments, the method comprises analyzing the biological sample to obtain the gene expression measurements of the biological sample. In certain embodiments, the method comprises analyzing the gene expression measurements to obtain the dataset. In certain embodiments, the method comprises obtaining and/or deriving the biological sample from the patient, and/or analyzing the biological sample to obtain the gene expression measurement of the biological sample. In certain embodiments, the method comprises obtaining and/or deriving the biological sample from the patient, analyzing the biological sample to obtain the gene expression measurement of the biological sample, and/or analyzing the gene expression measurements to obtain the dataset.

In certain embodiments, the data set is derived from the gene expression measurements using GSVA, gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof. In certain embodiments, the data set is derived from the gene expression measurements using GSVA. In certain embodiments, the data set is derived from the gene expression measurements using GSVA, wherein the data set comprises one or more GSVA scores of the patient, wherein each GSVA score is generated based on one of the one or more Tables selected from Tables 1 to 11, wherein for each selected Table, the genes selected from the selected Table forms the input gene set for generating the GSVA score based on the selected Table, using GSVA. In certain embodiments, the data set is derived from the gene expression measurements using GSVA, wherein the data set comprises one or more GSVA scores of the patient, wherein each GSVA score is generated based on one of the one or more gene clusters selected from Tables 15, 16, 17, 20, 21, or 22, wherein for each selected cluster, the genes selected from the selected cluster forms the input gene set for generating the GSVA score based on the selected Table, using GSVA. Enrichment of an input gene set based on a gene Table/cluster in the biological sample using GSVA can be determined to obtain the GSVA score based on the gene Table/cluster. In some embodiments, the GSVA score based on a selected Table can be generated based on enrichment of the genes selected from the selected Table (e.g., input gene set based on the selected Table) in the biological sample. In some embodiments, the GSVA score based on a selected cluster can be generated based on enrichment of the genes selected from the selected cluster (e.g., input gene set based on the selected cluster) in the biological sample. In a non-limiting example, Table 1, Table 2, and Table 3 are selected, the dataset comprises 3 or more GSVA scores, e.g., the dataset comprises a GSVA score generated based on Table 1, a GSVA score generated based on Table 2, and a GSVA score generated based on Table 3, wherein the GSVA score generated based on Table 1 is generated based on enrichment of the genes selected from the Table 1 (e.g., input gene set based on Table 1) in the biological sample, the GSVA score generated based on Table 2 is generated based on enrichment of the genes selected from the Table 2 in the biological sample, and the GSVA score generated based on Table 3 is generated based on enrichment of the genes selected from the Table 3 in the biological sample. The one or more Tables selected (e.g., based on which the one or more GSVA of the patient scores are generated) can comprise the Tables as described herein. For a selected Table the genes selected (e.g., that forms the input gene set for generating the GSVA score based on the selected Table) from the selected Table can comprise the selected genes as described herein, such as at least 2 genes, effective number of genes, and/or all genes from the selected Table. The GSVA scores can be GSVA enrichment scores, and can be generated using GSVA using the respective input gene sets. In certain embodiments, for each selected Table the genes selected (e.g., that forms the input gene set for generating the GSVA score based on the selected Table) comprise at least 2 genes selected from the genes listed in the selected Table, wherein a different or identical number of genes are selected from the genes listed in each selected table. In certain embodiments, for each selected Table the genes selected (e.g., that forms the input gene set for generating the GSVA score based on the selected Table) comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or all genes selected from the genes listed in the selected Table, wherein a different or identical number of genes are selected from the genes listed in each selected table. In certain embodiments, for each selected Table the genes selected (e.g., that forms the input gene set for generating the GSVA score based on the selected Table) comprise an effective number of genes selected from the genes listed in the selected Table, wherein a different or identical number of genes are selected from the genes listed in each selected table. In certain embodiments, for each selected Table the genes selected (e.g., that forms the input gene set for generating the GSVA score based on the selected Table) comprise all genes listed in the selected Table.

In certain embodiments, the effective number of genes for a Table can be determined using adjusted rand index (ARI) method. The ARI method can include performing k-Means clustering on randomly selected gene subsets by standard interval based on the total number of genes of a Table. Similarity between two clustering can be measured by adjusted rand index (ARI). As a non-limiting example, the adjusted rand index (ARI) can be calculated between k-Means cluster memberships from the randomly selected gene subsets to the cluster memberships obtained using total number of genes of the Table. The higher the ARI, the similar the cluster memberships and lower the ARI the weaker the cluster memberships, suggesting more genes may be required. The ARI can be calculated to determine the effective number of genes for each module. In certain embodiments, selecting effective number of genes from a Table (e.g., one of Tables 1 to 11) can include selecting at least 60%, 65%, 70%, 75%, 80%, 85%, 90%, 95%, or all genes from the Table. In certain embodiments, selecting effective number of genes from a Table (e.g., one of Tables 1 to 11) can include selecting at least 60% of the genes from the Table. In certain embodiments, selecting effective number of genes from a Table (e.g., one of Tables 1 to 11) can include selecting at least 70% of the genes from the Table. In certain embodiments, selecting effective number of genes from a Table (e.g., one of Tables 1 to 11) can include selecting at least 80% of the genes from the Table. In certain embodiments, selecting effective number of genes from a Table (e.g., one of Tables 1 to 11) can include selecting at least 90% of the genes from the Table. In certain embodiments, selecting effective number of genes from a Table (e.g., one of Tables 1 to 11) can include selecting all the genes from the Table.

In certain embodiments, Tables 1 to 11 are selected, wherein the dataset comprises a GSVA score based on Table 1, a GSVA score based on Table 2, a GSVA score based on Table 3, a GSVA score based on Table 4, a GSVA score based on Table 5, a GSVA score based on Table 6, a GSVA score based on Table 7, a GSVA score based on Table 8, a GSVA score based on Table 9, a GSVA score based on Table 10, and a GSVA score based on Table 11, and wherein the GSVA score based on Table 1 is generated based on enrichment of the genes selected from Table 1 (e.g., at least 2 genes, effective number of genes, and/or all genes selected from the genes listed in Table 1) in the biological sample, the GSVA score based on Table 2 is generated based on enrichment of the genes selected from Table 2 (e.g., at least 2 genes, effective number of genes, and/or all genes selected from the genes listed in Table 2) in the biological sample, the GSVA score based on Table 3 is generated based on enrichment of the genes selected from Table 3 (e.g., at least 2 genes, effective number of genes, and/or all genes selected from the genes listed in Table 3) in the biological sample, the GSVA score based on Table 4 is generated based on enrichment of the genes selected from Table 4 (e.g., at least 2 genes, effective number of genes, and/or all genes selected from the genes listed in Table 4) in the biological sample, the GSVA score based on Table 5 is generated based on enrichment of the genes selected from Table 5 (e.g., at least 2 genes, effective number of genes, and/or all genes selected from the genes listed in Table 5) in the biological sample, the GSVA score based on Table 6 is generated based on enrichment of the genes selected from Table 6 (e.g., at least 2 genes, effective number of genes, and/or all genes selected from the genes listed in Table 6) in the biological sample, the GSVA score based on Table 7 is generated based on enrichment of the genes selected from Table 7 (e.g., at least 2 genes, effective number of genes, and/or all genes selected from the genes listed in Table 7) in the biological sample, the GSVA score based on Table 8 is generated based on enrichment of the genes selected from Table 8 (e.g., at least 2 genes, effective number of genes, and/or all genes selected from the genes listed in Table 8) in the biological sample, the GSVA score based on Table 9 is generated based on enrichment of the genes selected from Table 9 (e.g., at least 2 genes, effective number of genes, and/or all genes selected from the genes listed in Table 9) in the biological sample, the GSVA score based on Table 10 is generated based on enrichment of the genes selected from Table 10 (e.g., at least 2 genes, effective number of genes, and/or all genes selected from the genes listed in Table 10) in the biological sample, and the GSVA score based on Table 11 is generated based on enrichment of the genes selected from Table 11 (e.g., at least 2 genes, effective number of genes, and/or all genes selected from the genes listed in Table 11) in the biological sample. In certain embodiments, Tables 1 to 11 are selected, and for each selected Tables all genes listed in the selected Table are selected, wherein the dataset comprises a GSVA score based on Table 1, a GSVA score based on Table 2, a GSVA score based on Table 3, a GSVA score based on Table 4, a GSVA score based on Table 5, a GSVA score based on Table 6, a GSVA score based on Table 7, a GSVA score based on Table 8, a GSVA score based on Table 9, a GSVA score based on Table 10, and a GSVA score based on Table 11, and wherein the GSVA score based on Table 1 is generated based on enrichment of the genes listed in Table 1 in the biological sample, the GSVA score based on Table 2 is generated based on enrichment of the genes listed in Table 2 in the biological sample, the GSVA score based on Table 3 is generated based on enrichment of the genes listed in Table 3 in the biological sample, the GSVA score based on Table 4 is generated based on enrichment of the genes listed in Table 4 in the biological sample, the GSVA score based on Table 5 is generated based on enrichment of the genes listed in Table 5 in the biological sample, the GSVA score based on Table 6 is generated based on enrichment of the genes listed in Table 6 in the biological sample, the GSVA score based on Table 7 is generated based on enrichment of the genes listed in Table 7 in the biological sample, the GSVA score based on Table 8 is generated based on enrichment of the genes listed in Table 8 in the biological sample, the GSVA score based on Table 9 is generated based on enrichment of the genes listed in Table 9 in the biological sample, the GSVA score based on Table 10 is generated based on enrichment of the genes listed in Table 10 in the biological sample, and the GSVA score based on Table 11 is generated based on enrichment of the genes listed in Table 11 in the biological sample.

The one or more GSVA scores of the patient, can be generated based on comparing gene expression measurements of the biological sample obtained and/or derived from the patient, with gene expression measurements from a reference dataset. The reference data set can comprise and/or be derived from gene expression measurements from a plurality of reference biological samples. The plurality of reference biological samples can be obtained or derived from a plurality of reference subjects. In certain embodiments, at least a portion of the reference subjects have lupus. In certain embodiments, at least a first portion of the reference subjects have lupus, and is of Asian ancestry, and at least a second portion of the reference subjects have lupus, and is of European ancestry. In certain embodiments, at least a first portion of the reference subjects have lupus, and is of East Asian (e.g., Chinese) ancestry, and at least a second portion of the reference subjects have lupus, and is of European ancestry. In certain embodiments, the plurality of reference biological samples comprise a first plurality of the reference biological samples obtained or derived from reference subjects having lupus, and/or a second plurality of the reference biological samples obtained or derived from reference subjects not having lupus. In certain embodiments, the plurality of reference biological samples comprise a first plurality of the reference biological samples obtained or derived from reference subjects having lupus and is of Asian ancestry, a second plurality of the reference biological samples obtained or derived from reference subjects having lupus and is of European ancestry, and/or a third plurality of reference subjects not having lupus. In certain embodiments, the plurality of reference biological samples comprise a first plurality of the reference biological samples obtained or derived from reference subjects having lupus and is of East Asian ancestry, a second plurality of the reference biological samples obtained or derived from reference subjects having lupus and is of European ancestry, and/or a third plurality of reference subjects not having lupus. In certain embodiments, the reference data set comprise and/or is derived from gene expression measurements from the plurality of reference biological samples of at least 2 genes selected from the genes listed in each of one or more Tables selected from Tables: 1 to 11. In certain embodiments, the reference data set comprise and/or is derived from gene expression measurements from the plurality of reference biological samples of all the genes listed in each of one or more Tables selected from Tables: 1 to 11. The selected genes of the dataset (e.g., gene expression measurements of which the dataset is comprised of or derived from), and the selected genes of the reference data set (e.g., gene expression measurements of which the reference dataset is comprised of or derived from) can at least partially overlap (e.g., one or more of the selected genes can be the same). In certain embodiments, selected genes of the dataset, and selected genes of the reference data are same. In certain embodiments, selected genes of the dataset, and selected genes of the reference data are same, and can be any selected gene set, e.g., of the data set, as described herein. The enrichment of the input gene sets in the biological sample can be determined (e.g., for determining the one or more GSVA scores of the patient) based on comparing the gene expression measurements from the biological sample obtained and/or derived from the patient, with the gene expression measurements from the plurality of reference biological samples of the reference dataset. In certain embodiments, the reference data set can be a reference data set as described in the Example.

Analyzing the data set can include determining whether a set of genes selected from a selected Table, are enriched in the biological sample, wherein the one or more sets of genes enriched in the biological sample can comprise the sets of genes that are enriched in the biological sample. The genes selected from each selected Table can form a set of genes selected from the selected Table, wherein genes selected from same selected Table can be part of a same set of genes, and genes selected from different selected Tables can form different sets of genes. As a non-limiting example, Table 1 and Table 2 can be selected from Tables 1 to 11, and genes selected from Table 1 can form a set of genes, and genes selected from Table 2 can form another set of genes.

The patient may be diagnosed with lupus if a set of genes selected from any of the selected Tables or clusters are enriched in the biological sample, e.g., the one or more sets of genes comprises a set of gene selected from a selected Table or cluster. In some embodiments, the patient is diagnosed with lupus if a set of genes selected from any of the selected Tables from Tables 1 to 11 are enriched in the biological sample, e.g., the one or more sets of genes comprises a set of gene selected from a selected Table. In some embodiments, the patient is diagnosed with lupus if a set of genes selected from any of the selected clusters from Table 15G and/or 15H are enriched in the biological sample, e.g., the one or more sets of genes comprises a set of genes selected from a selected cluster. Enrichment can be relative to, e.g., a non-lupus control. A set of genes selected from a selected Table can be considered enriched if the set of genes as a group is enriched in the biological sample from the patient relative to non-lupus control reference subjects. Enrichment of the set of genes as a group in the biological sample can be measured using GSVA, GSEA, enrichment algorithm, MEGENA, WGCNA, differential expression analysis, Z-score, log 2 expression analysis, or any combination thereof. In certain embodiments, the enrichment of a set of genes can be measured using a Z-score. In certain embodiments, a set of genes can be considered enriched in the biological sample from the patient, when Z-score of the patient for the set of genes, is greater than 0.1, 0.5, 1, 1.5, 2, 2.5, or 3. In certain embodiments, a set of genes can be considered enriched in the biological sample from the patient, when the Z-score of the patient for the gene feature, is greater than 2. The Z-score of the patient for a gene feature can be calculated as, =(GSVA score of the set of genes of the patient-mean GSVA score of the set of genes for non-lupus controls)/standard deviation of the GSVA scores of the set of genes for non-lupus controls. GSVA score of the set of genes of the patient, can be a GSVA score generated using the set of genes as input gene set for GSVA, e.g., a GSVA score generated based on enrichment of the set of genes in the biological sample from the patient. Mean GSVA score and the standard deviation for non-lupus controls can be calculated based on gene expressions measurements from reference samples from non-lupus controls reference subjects of a reference dataset described herein. The reference dataset based on which the GSVA score of the patient is determined, and reference dataset based on which the mean GSVA score and the standard deviation for non-lupus controls are calculated can be the same.

In certain embodiments, analyzing the data set comprises providing the data set as an input to a trained machine-learning model trained to generate an inference of whether the data set is indicative of the patient having lupus. The inference can be indicative of the one or more sets of genes enriched in the biological sample. In certain embodiments, the method further comprises receiving, as an output of the trained machine-learning model, the inference; and/or electronically outputting a report classifying the lupus disease state of a patient.

The trained machine-learning model can be trained using linear regression, logistic regression (LOG), Ridge regression, Lasso regression, an elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

The trained machine-learning model can generate the inference, based on comparing the data set to a reference data set. The trained machine-learning model can be trained using the reference dataset. The reference data set can comprise and/or be derived from gene expression measurements from a plurality of reference biological samples. The plurality of reference biological samples can be obtained or derived from a plurality of reference subjects. In some embodiments, the plurality of reference subjects comprise a first plurality of reference subjects having lupus, and second plurality of reference subjects not having lupus. The one or more GSVA scores of the patient, can be generated based on comparing gene expression measurements of the biological sample obtained and/or derived from the patient, with the gene expression measurements of the plurality reference biological samples, of the reference dataset. The enrichment of the input gene sets in the biological sample can be determined (e.g., for determining the one or more GSVA scores of the patient) based on comparing the gene expression measurements from the biological sample obtained and/or derived from the patient, with the gene expression measurements from the reference biological samples of the reference dataset.

In certain embodiments, the method further comprises recommending, selecting, and/or administering a treatment to the patient based on the enrichment of the one or more sets of genes. In certain embodiments, the method further comprises administering a treatment to the patient based on the enrichment of the one or more sets of genes. In certain embodiments, the treatment is configured to treat lupus. In certain embodiments, the treatment is configured to reduce severity of lupus. In certain embodiments, the treatment is configured to reduce risk of having lupus. In certain embodiments, the treatment can be based on a functional annotation of a Table selected from Tables 1 to 11, wherein the set of genes selected from the Table is enriched in the biological sample, e.g., the one or more sets of genes comprise the set of genes selected from the selected Table. In certain embodiments, the treatment can be based on a functional annotation of a gene cluster selected from the gene clusters listed in Tables 15, 16, 17, 20, 21, or 22, wherein the set of genes selected from the gene cluster is enriched in the biological sample, e.g., the one or more sets of genes comprise the set of genes selected from the selected gene cluster. The functional annotations of the Tables/clusters may be determined using a functional annotation method as described in WO2021/231713, “Methods and Systems for Machine Learning Analysis of Single Nucleotide Polymorphisms in Lupus,” which is incorporated herein by reference in its entirety. As a non-limiting example only: Tables 1 to 11 are selected, and all genes listed in each of the selected Tables are selected, i.e., the dataset comprises or is derived from gene expression measurements of all the genes from each of Tables 1 to 11; analysis of the data set according to the method may determine genes selected from Table 1 are enriched in the biological sample, i.e., the set of genes enriched in a biological sample can comprise genes selected from Table 1; and the treatment administered can target the JAK signaling pathway. The treatment may or may not target all the genes enriched in the biological sample, for example the set of genes enriched in a biological sample may comprise genes selected from Table 1, and Table 2, wherein the treatment may target the JAK signaling pathway, the oxidative phosphorylation pathway, or both. A treatment targeting a pathway may down regulate genes associated with and/or downstream of the pathway.

In certain embodiments, the treatment targets the JAK signaling pathway, the oxidative phosphorylation pathway, the sirtuin signaling pathway, the mitochondrial dysfunction pathway, the glycolysis pathway, the reactive oxygen species (ROS) protection pathway, the MTOR signaling pathway, the microRNA processing pathway, the TNF signaling pathway, or any combination thereof.

In certain embodiments, the treatment comprises baricitinib, carfilzomib, curcumol, decernotinib, delgocitinib, ruxolitinib, solicitinib, tofacitinib, upadacitinib, bortezomib, densosumab, filgotinib, idelalisib, KZR-616, peficitinib, metformin, phenformin, BAY84-2243, CAI, ME344, fenofibrate, lonidamine, arsenic trioxide, atovaquone, hydrocortisone, a-TOS, thapsigargin, resveratrol, cyclosporin A, N-acetyl L-cysteine, SKQ1, ubiquinone, mito VitE, mitoTEMPO, vitamin E, vitamin C, ALT-2074, Ebselen, GC4419, sirolimus, everolimus, temsirolimus, thapsigargin, adalimumab, AMG-811, baricitinib, BMS-986165, certolizumab, dacomitinib, etanercept, filgotinib, iguratimod, infliximab, ruxolitinib, solicitinib, tabalumab, trofinetide, upadacitinib, or any combinations thereof.

In certain embodiments, the treatment for enrichment of the genes selected from the Table 1, targets JAK signaling pathway; treatment for enrichment of the genes selected from the Table 2, targets oxidative phosphorylation pathway; treatment for enrichment of the genes selected from the Table 3, targets sirtuin signaling pathway; treatment for enrichment of the genes selected from the Table 4, targets mitochondrial dysfunction pathway; treatment for enrichment of the genes selected from the Table 5, targets glycolysis pathway; treatment for enrichment of the genes selected from the Table 6, targets reactive oxygen species (ROS) protection pathway, treatment for enrichment of the genes selected from the Table 7, targets MTOR signaling pathway; treatment for enrichment of the genes selected from the Table 8, targets JAK signaling pathway; treatment for enrichment of the genes selected from the Table 9, targets microRNA processing pathway; treatment for enrichment of the genes selected from the Table 10, targets mitochondrial dysfunction pathway; and/or treatment for enrichment of the genes selected from the Table 11, targets TNF signaling pathway. In certain embodiments, the treatment targeting the JAK signaling pathway comprises a JAK inhibitor. In certain embodiments, the treatment targeting the MTOR signaling pathway comprises a MTOR inhibitor. In certain embodiments, the treatment targeting the TNF signaling pathway comprises a TNF inhibitor. In certain embodiments, the treatment targeting the JAK signaling pathway comprises baricitinib, carfilzomib, curcumol, decernotinib, delgocitinib, ruxolitinib, solicitinib, tofacitinib, upadacitinib, bortezomib, densosumab, filgotinib, idelalisib, KZR-616, peficitinib, or any combination thereof. In certain embodiments, the treatment targeting the oxidative phosphorylation pathway comprises metformin, phenformin, BAY84-2243, CAI, ME344, fenofibrate, lonidamine, arsenic trioxide, atovaquone, hydrocortisone, a-TOS, thapsigargin, or any combination thereof. In certain embodiments, the treatment targeting the sirtuin signaling pathway comprises resveratrol, and/or cyclosporin A. In certain embodiments, the treatment targeting the mitochondrial dysfunction pathway comprises resveratrol, N-acetyl L-cysteine, SKQ1, ubiquinone, mitoVitE, mitoTEMPO, vitamin E, vitamin C, or any combination thereof. In certain embodiments, the treatment targeting the glycolysis pathway comprises Cylcosporin A. In certain embodiments, the treatment targeting the reactive oxygen species (ROS) protection pathway comprises resveratrol, N-acetyl L-cysteine, SKQ1, ubiquinone, mito VitE, mitoTEMPO, vitamin E, vitamin C, ALT-2074, Ebselen, GC4419, or any combination thereof. In certain embodiments, the treatment targeting the MTOR signaling pathway comprises sirolimus, everolimus, temsirolimus, or any combination thereof. In certain embodiments, the treatment targeting the microRNA processing pathway comprises cyclosporin A, and/or thapsigargin. In certain embodiments, the treatment targeting the TNF signaling pathway comprises adalimumab, AMG-811, baricitinib, BMS-986165, certolizumab, dacomitinib, etanercept, filgotinib, iguratimod, infliximab, ruxolitinib, solicitinib, tabalumab, trofinetide, upadacitinib, or any combinations thereof.

The biological sample can comprises a blood sample, isolated peripheral blood mononuclear cells (PBMCs), a tissue biopsy sample, or any derivative thereof. In certain embodiments, the biological sample comprise a blood sample, or any derivative thereof. In certain embodiments, the biological sample comprise a PBMCs, or any derivative thereof. In certain embodiments, the biological sample comprise a tissue biopsy sample, or any derivative thereof. In certain embodiments, the patient has lupus. In certain embodiments, the patient is at elevated risk of having lupus. In certain embodiments, the patient is suspected of having lupus. In certain embodiments, the patient is asymptomatic for lupus. In certain embodiments, the patient is of Asian ancestry. In certain embodiments, the patient is of European ancestry.

In certain embodiments, the method further comprises monitoring the lupus disease state of the patient, wherein the monitoring comprises assessing the lupus disease state of the patient at a plurality of different time points. A difference in the assessment of the lupus disease state of the patient among the plurality of time points can be indicative of one or more clinical indications selected from the group consisting of: (i) a diagnosis of the lupus disease state of the patient, (ii) a prognosis of the lupus disease state of the patient, and (iii) an efficacy or non-efficacy of a course of treatment for treating the lupus disease state of the patient. In certain embodiments, the patient has been administered a treatment, and the method can assess an efficacy or non-efficacy of the treatment, for treating the lupus disease state of the patient.

Lupus can be any type of lupus including but not limited to systemic lupus erythematosus (SLE), cutaneous lupus erythematosus, drug-induced lupus, and neonatal lupus. In certain embodiments lupus can be SLE.

Certain aspects, are directed to a biomarker assay developed according to a method described herein. Certain aspects, are directed to a kit comprising the biomarker assay developed according to a method described herein, and/or a biomarker assay of described herein.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a head-mounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for identifying one or more records having a specific phenotype. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

### Biological Data Analysis

Certain embodiments, of the present disclosure provides systems and methods to perform data analysis using drug or target scoring algorithms and/or big data analysis tools. In various aspects, such drug or target scoring algorithms and/or big data analysis tools may be used to perform analysis of data sets including, for example, mRNA gene expression or transcriptome data, DNA genomic data, proteomic data, metabolomic data, other types of “-omic” data, or a combination thereof.

In an aspect, the present disclosure provides a computer-implemented method for assessing a condition of a subject, comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject.

In some embodiments, the dataset comprises mRNA gene expression or transcriptome data, DNA genomic data, proteomic data, metabolomic data, or a combination thereof. In some embodiments, the biological sample is selected from the group consisting of: a whole blood (WB) sample, a PBMC sample, a tissue sample, and a cell sample. In some embodiments, assessing the condition of the subject comprises identifying a disease or disorder of the subject.

In some embodiments, the method further comprises identifying a disease or disorder of the subject at a sensitivity or specificity of at least about 70%. In some embodiments, the method further comprises determining a likelihood of the identification of the disease or disorder of the subject. In some embodiments, the method further comprises providing a therapeutic intervention for the disease or disorder of the subject. In some embodiments, the method further comprises monitoring the disease or disorder of the subject, wherein the monitoring comprises assessing the disease or disorder of the subject at a plurality of time points, wherein the assessing is based at least on the disease or disorder identified at each of the plurality of time points.

In some embodiments, selecting the one or more data analysis tools comprises receiving a user selection of the one or more data analysis tools. In some embodiments, selecting the one or more data analysis tools is automatically performed by the computer without receiving a user selection of the one or more data analysis tools.

In another aspect, the present disclosure provides a computer system for assessing a condition of a subject, comprising: a database that is configured to store a dataset of a biological sample of the subject; and one or more computer processors operatively coupled to the database, wherein the one or more computer processors are individually or collectively programmed to: (i) select one or more data analysis tools comprising: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, a Target Scoring analysis tool, or a combination thereof; (ii) process the dataset using the one or more data analysis tools to generate a data signature of the biological sample of the subject; and (iii) based at least in part on the data signature generated in (ii), assess the condition of the subject.

In another aspect, the present disclosure provides a non-transitory computer readable medium comprising machine-executable code that, upon execution by one or more computer processors, implements a method for assessing a condition of a subject, the method comprising: (a) receiving a dataset of a biological sample of the subject; (b) selecting one or more data analysis tools, wherein the one or more data analysis tools comprise an analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool; (c) processing the dataset using the one or more data analysis tools to generate a data signature of the biological sample of the subject; and (d) based at least in part on the data signature generated in (c), assessing the condition of the subject. In any embodiment described herein, the one or more data analysis tools may be a plurality of data analysis tools each independently selected from a BIG-C™ big data analysis tool, an I-Scope™ big data analysis tool, a T-Scope™ big data analysis tool, a CellScan big data analysis tool, an MS (Molecular Signature) Scoring™ analysis tool, a Gene Set Variation Analysis (GSVA) tool (e.g., P-Scope), a CoLTs® (Combined Lupus Treatment Scoring) analysis tool, and a Target Scoring analysis tool.

To obtain a blood sample, various techniques may be used, e.g., a syringe or other vacuum suction device. A blood sample may be optionally pre-treated or processed prior to use. A sample, such as a blood sample, may be analyzed under any of the methods and systems herein within 4 weeks, 2 weeks, 1 week, 6 days, 5 days, 4 days, 3 days, 2 days, 1 day, 12 hr, 6 hr, 3 hr, 2 hr, or 1 hr from the time the sample is obtained, or longer if frozen. When obtaining a sample from a subject (e.g., blood sample), the amount may vary depending upon subject size and the condition being screened. In some embodiments, at least 10 mL, 5 mL, 1 mL, 0.5 mL, 250, 200, 150, 100, 50, 40, 30, 20, 10, 9, 8, 7, 6, 5, 4, 3, 2, or 1 μL of a sample is obtained. In some embodiments, 1-50, 2-40, 3-30, or 4-20 μL of sample is obtained. In some embodiments, more than 5, 10, 15, 20, 25, 30, 35, 40, 45, 50, 55, 60, 65, 70, 75, 80, 85, 90, 95 or 100 μL of a sample is obtained.

The sample may be taken before and/or after treatment of a subject with a disease or disorder. Samples may be obtained from a subject during a treatment or a treatment regime. Multiple samples may be obtained from a subject to monitor the effects of the treatment over time. The sample may be taken from a subject known or suspected of having a disease or disorder for which a definitive positive or negative diagnosis is not available via clinical tests. The sample may be taken from a subject suspected of having a disease or disorder. The sample may be taken from a subject experiencing unexplained symptoms, such as fatigue, nausea, weight loss, aches and pains, weakness, or bleeding. The sample may be taken from a subject having explained symptoms. The sample may be taken from a subject at risk of developing a disease or disorder due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

In some embodiments, a sample may be taken at a first time point and assayed, and then another sample may be taken at a subsequent time point and assayed. Such methods may be used, for example, for longitudinal monitoring purposes to track the development or progression of a disease. In some embodiments, the progression of a disease may be tracked before treatment, after treatment, or during the course of treatment, to determine the treatment's effectiveness.

For example, a method as described herein may be performed on a subject prior to, and after, treatment with a first, second, and/or third disease condition therapy to measure the disease's progression or regression in response to the first, second, and/or third disease condition therapy. The first, second, and/or third disease can be as described above.

After obtaining a sample from the subject, the sample may be processed to generate datasets indicative of a disease or disorder of the subject. For example, a presence, absence, or quantitative assessment of nucleic acid molecules of the sample from a panel of condition-associated genomic loci or nucleotide polymorphism may be indicative of first, second, and/or third disease condition of the subject. Processing the sample obtained from the subject may comprise (i) subjecting the sample to conditions that are sufficient to isolate, enrich, or extract a plurality of nucleic acid molecules, and (ii) assaying the plurality of nucleic acid molecules to generate the dataset (e.g., microarray data, nucleic acid sequences, or quantitative polymerase chain reaction (qPCR) data). Methods of assaying may include any assay known in the art or described in the literature, for example, a microarray assay, a sequencing assay (e.g., DNA sequencing, RNA sequencing, or RNA-Seq), or a quantitative polymerase chain reaction (qPCR) assay.

In some embodiments, a plurality of nucleic acid molecules is extracted from the sample and subjected to sequencing to generate a plurality of sequencing reads. The nucleic acid molecules may comprise ribonucleic acid (RNA) or deoxyribonucleic acid (DNA). The extraction method may extract all RNA or DNA molecules from a sample. Alternatively, the extraction method may selectively extract a portion of RNA or DNA molecules from a sample. Extracted RNA molecules from a sample may be converted to cDNA molecules by reverse transcription (RT).

The sample may be processed without any nucleic acid extraction. For example, the disease or disorder may be identified or monitored in the subject by using probes configured to selectively enrich nucleic acid (e.g., RNA or DNA) molecules corresponding to a panel of condition-associated genomic loci. The probes may be nucleic acid primers. The probes may have sequence complementarity with nucleic acid sequences from one or more of the panel of condition-associated genomic loci. The panel of condition-associated genomic loci may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more condition-associated genomic loci.

The probes may be nucleic acid molecules (e.g., RNA or DNA) having sequence complementarity with nucleic acid sequences (e.g., RNA or DNA) of one or more genomic loci (e.g., condition-associated genomic loci). These nucleic acid molecules may be primers or enrichment sequences. The assaying of the sample using probes that are selective for the one or more genomic loci (e.g., condition-associated genomic loci) may comprise use of array hybridization, polymerase chain reaction (PCR), or nucleic acid sequencing (e.g., RNA sequencing or DNA sequencing, such as RNA-Seq).

The assay readouts may be quantified at one or more genomic loci (e.g., condition-associated genomic loci) to generate the data indicative of the disease or disorder. For example, quantification of array hybridization or polymerase chain reaction (PCR) corresponding to a plurality of genomic loci (e.g., condition-associated genomic loci) may generate data indicative of the disease or disorder. Assay readouts may comprise quantitative PCR (qPCR) values, digital PCR (dPCR) values, digital droplet PCR (ddPCR) values, fluorescence values, etc., or normalized values thereof.

The BIG-C (Biologically Informed Gene Clustering) tool may be configured to sort large groups of genes into a set of functional groups (e.g., 53 functional groups). The functional groups are created utilizing publicly available information from online tools and databases including UniProtKB/Swiss-Prot, GO Terms, KEGG pathways, NCBI PubMed, and the Interactome. The functional groups may include one or more of: Active RNA, Anti-apoptosis, anti-proliferation, autophagy, chromatin remodeling, cytoplasm and biochemistry, cytoskeleton, DNA repair, endocytosis, endoplasmic reticulum, endosome and vesicles, fatty acid biosynthesis, cell surface, transcription, glycolysis and gluconeogenesis, golgi, immune cell surface, immune secreted, immune signaling, integrin pathway, interferon stimulated genes, intracellular signaling, lysosome, melanosome, MHC class I, MHC class II, microRNA processing, microRNA, mitochondrial transcription, mitochondria, mitochondria oxidative phosphorylation, mitochondrial TCA cycle, mRNA processing, mRNA splicing, non-coding RNA, nuclear receptor, nucleus and nucleolus, palmitoylation, pattern recognition receptors, peroxisomes, pro-apoptosis, pro-cell cycle, proteasome, pseudogenes, RAS superfamily, reactive oxygen species protection, secreted and extracellular matrix, transcription factors, transporters, transposon control, ubiquitylation and sumoylation, unfolded protein and stress, and unknown. Enrichment scores for each group are calculated based on an overlap p value to determine the functional groups over or under-expressed in the gene expression dataset. The BIG-C may be configured such that each gene is sorted into only one of the 53 functional groups, allowing for a quick and relatively simple understanding of types of genes enriched and co-expressed in a big dataset.

The I-Scope™ tool may be configured to identify immune infiltrates. Hematopoietic cells are unique in that they move throughout the body patrolling for threats to the host, and may infiltrate tissue sites not normally home to immune cells. I-Scope™ may be configured to identify hematopoietic cells through an iterative search of more than 17,000 genes identified in more than 50 microarray datasets. From this search, 1226 candidate genes are identified and researched for restriction in hematopoietic cells as determined by the HPA, GTEx and FANTOM5 datasets (e.g., available at proteinatlas.org). 926 genes meet the criteria for being mainly restricted to hematopoietic lineages (brain, reproductive organ exclusions were permitted). These genes are researched for immune cell specific expression in 27 hematopoietic sub-categories: alpha beta T cell, T cell, regulatory T Cell, activated T cell, anergic T cell, gamma delta T cells, CD8 T, NK/NKT cell, NK cell, T & B cells, B cells, germinal center B cells, B cell and plasmacytoid dendritic cell, T &B & myeloid, B & myeloid, T & myeloid, MHC Class II expressing cell, monocyte, dendritic cell, plasmacytoid dendritic cells, myeloid cell, plasma cell, erythrocyte, neutrophil, low density granulocyte, granulocyte, and platelet. Transcripts are entered into I-Scope™ and the number of transcripts in each category determined. Odd's ratios are calculated with confidence intervals using the Fisher's exact test in R.

The T-Scope™ tool may be configured to help identify types of non-hematopoietic cells in gene expression datasets. T-Scope™ may be configured by downloading approximately 10,000 tissue enriched and 8,000 cell line enriched genes from the human protein atlas along with their tissue or cell line designation (e.g., available at proteinatlas.org). Genes found in more than four tissues are eliminated. Housekeeping genes described in the gene expression study by She et al. are also removed (e.g., as described by She et al., “Definition, conservation and epigenetics of housekeeping and tissue-enriched genes,” BMC Genomics 2009, 10:269, which is incorporated herein by reference in its entirety). This list is further curated by removing genes differentially expressed in 34 hematopoietic cell gene expression datasets and adding kidney specific genes from datasets downloaded from the GEO repository and processed by Ampel BioSolutions. The resulting categories of genes represent genes enriched in the following 42 tissue/cell specific categories: adrenal gland, breast, cartilage, cerebral cortex, uterine cervix, chondrocyte, colon, duodenum, endometrium, epididymis, esophagus fallopian tube, esophagus, fibroblast, heart muscle, keratinocyte, kidney, liver, lung, melanocyte, ovary pancreas, parathyroid gland, placenta, podocyte, prostrate, rectum, salivary gland, seminal vesicle, skeletal muscle, skin, small intestine, smooth muscle, stomach, synoviocyte, testis, kidney loop of henle, kidney proximal tubule, kidney distal tubule, and kidney collecting duct.

The CellScan tool may be a combination of I-Scope™ and T-Scope™, and may be configured to analyse tissues with suspected immune infiltrations that may also have tissue specific genes. CellScan may potentially be more stringent than either I-Scope™ or T-Scope™ because it may be used to distinguish resident tissue cells from non-resident hematopoietic cells.

The MS (Molecular Signature) Scoring tool may be configured to assess specific pathways in a disease state. Information on genes that encode for proteins that participate in a specific signaling pathway, and whether the gene product promotes or inhibits the pathway, are compiled and curated through literature mining. Curated pathways presented by the company include CD40-CD40ligand, IL-6, IL-12/23, TNF, IL-17, IL-21, SIP1, IL-13 and PDE4, but this method may be used for any known signaling pathway with available data. To determine if a signaling pathway is over or under-expressed in a microarray dataset, the gene list for each signaling pathway may be queried against the limma differentially expressed genes from a disease state compared to healthy controls, and the differentially expressed genes in the signaling pathway may be identified for each set. The fold changes for genes that promoted the pathway may be added together and the fold changes for genes that inhibited the pathway may be subtracted from the score. This total score may be normalized based on the number of genes that may be detected on the specific microarray platform used for the experiment. Activation scores of −100 to +100 may be determined using this method with negative scores indicating an inhibition of the specific pathway in the disease state and positive scores indicating an up-regulation of a specific pathway in the disease state. The Fischer's exact test may be performed to determine if there was sufficient overlap of genes between the experimental differentially expressed genes and the genes in the signaling pathway.

Gene Set Variation Analysis (GSVA) may be performed (for example, as described in Catalina et al. (2019, Communications Biology, “Gene expression analysis delineates the potential roles of multiple interferons in systemic lupus erythematosus”, which is incorporated herein by reference in its entirety) to determine enrichment of signaling pathways in individual patient samples. Gene set variation analysis may be performed using an open source software package for the coding language R available at the R Bioconductor (bioconductor.org), e.g., as described by Hanzelman et al., (“GSVA: gene set variation analysis for microarray and RNA-Seq data,” BMC Bioinformatics, 2013, which is incorporated herein by reference in its entirety). The modules of genes to interrogate the datasets may be developed. Modules of genes determined to represent a specific signaling pathway or process may be identified (e.g., using publicly available datasets). For example, the IFNB1 signaling pathway is taken from a publicly available gene expression dataset of peripheral blood cells treated with IFNB1 in vitro. Genes co-expressed in this dataset (genes either all increased or decreased compared to control treated peripheral blood) are used to create modules of genes representing the IFNB1 signaling pathway, and GSVA is used to determine the enrichment of this set of genes and hence the IFNB1 signaling pathway in individual patient and control samples.

The CoLTs®, or Combined Lupus Treatment Scoring, may be configured to rank identified drugs or therapies by a number of essential characteristics, including scientific rationale, experience in lupus mice/human cells (preclinical), previous clinical experience in autoimmunity, drug properties, and safety profile, including adverse events. Face and test validities may be established by scoring SOC medications and confirming the scores with a panel of lupus clinicians. The final result may be the CoLTs® score. A CoLTs® algorithm may also be configured for drugs in development (DID), which typically do not have drug metabolism and adverse event information available.

The target scoring algorithm may be configured to prioritize a specific gene or protein that is potentially a good choice to target with a drug in first, second and/or third disease patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

### Big-C™ Big Data Analysis Tool

BIG-CR is a fast and efficient cloud-based tool to functionally categorize gene products. With coverage of over 80% of the genome, BIG-CR leverages publicly available databases such as UniProtKB/Swiss-Prot, GO terms, KEGG pathways, NCBI PubMed and Interactome to place genes into 53 functional categories. The sorting into only one of 53 functional groups allows for a quick and relatively simple understanding of types of genes enriched and co-expressed in a big dataset. This assists in deriving further insights from genes expressed for a given disease state in human or pre-clinical mouse models.

BIG-CR may be used to functionally categorize immunological genes that are not covered in cancer databases such as GO and KEGG (e.g., as described by Grammer et al. 2016, “Drug repositioning in SLE: crowd-sourcing, literature-mining and Big Data analysis,” Lupus, 25(10), 1150-1170, which is incorporated herein by reference in its entirety). Using a knowledge base of over 5000 patients with systemic lupus erythematosus (SLE), over 16432 genes are each placed into one of 53 BIG-C® functional categories, and statistical analysis is performed to identify enriched categories. BIG-CR categories are cross-examined with the GO and KEGG terms to obtain additional information and insights.

A sample BIG-CR workflow may comprise the following steps. First, SLE genomic datasets are derived from whole blood, peripheral blood mononuclear cells, affected tissues, and purified immune cells. Second, datasets are analyzed using DE analysis (as shown by a differential expression heatmap) or Weighted Gene Coexpression Network Analysis (WGCNA) (as shown by a gene coexpression plot). Third, expressed genes are annotated using publicly available databases (e.g., UniProtKB/Swiss-Prot database, Human Immunodeficiencies database, Mouse MGI database, Entrez Molecular Sequence database, PubMed, and the Human Tissue Atlas). Fourth, signatures are cross-referenced with purified single-cell microarray datasets and RNAseq experiments. Fifth, BIG-CR is leveraged to separate the individual annotated genes into one of 53 functional categories (e.g., as described by Labonte et al. 2018, “Identification of alterations in macrophage activation associated with disease activity in systemic lupus erythematosus,” PloS one, 13 (12), e0208132, which is incorporated herein by reference in its entirety). Sixth, chi-squared analysis is used to determine enriched categories of interest from overlap p-values. Seventh, enriched categories are cross-examined with GO and KEGG terms to derive key insights for further analysis.

### I-Scope™ Big Data Analysis Tool

I-Scope™ may be a tool configured for cross-examining the presence and activity of varying types of immune cell infiltrates with observed gene expression patterns. It may take annotated gene expression data and analyze it for hematopoietic cell lineage. I-Scope™ may be used downstream of the BIG-CR (Biologically Informed Gene-Clustering) tool in that it helps to provide even more insight into the nature of the genes being expressed after categorization.

I-Scope™ addresses the need to understand the involvement of specific cells for a given disease state. While it is helpful to understand the relative up-regulation and down-regulation at the gene expression level, it is even more informative to understand specifically in which cells this is occurring. I-Scope™ may be configured to identify hematopoietic cells through an iterative search of more than 17,000 genes identified in more than 50 microarray datasets (e.g., as described by Hubbard et al., “Analysis of Lupus Synovitis Gene Expression Reveals Dysregulation of Pathogenic Pathways Activated within Infiltrating Immune Cells,” Arthritis Rheumatol, 2018; 70 (suppl 10), which is incorporated herein by reference in its entirety). I-Scope™ may function by restricting the analysis to genes of hematopoietic cell heritage and allow for cross-checking against purified single-cell experiments or datasets. The cross-check confirms and categorizes specific transcript signatures to the 28 hematopoietic cell sub-categories, ultimately allowing for cellular activity analysis across multiple samples and disease states. When combined with BIG-C® categories, the cellular activity may be correlated to specific functions within a given cell type.

A sample I-Scope™ workflow may comprise the following steps. First, candidate genes are identified from SLE (systemic lupus erythematosus) datasets potentially associated with immune cell expression. Second, using HPA, GTEx, and FANTOM5 datasets, expression signatures associated with hematopoietic cell lineage are identified. Third, signatures are cross-referenced with purified single-cell microarray datasets and RNAseq experiments. Fourth, transcripts are categorized into 28 hematopoietic cell sub-categories and assess cellular expression across different samples and disease states. Odd's ratios are calculated with confidence intervals using the Fisher's exact test in R. An I-Scope™ signature analysis for a given sample may lead to the I-Scope™ signature analysis across multiple samples and disease states.

### T-Scope™ Big Data Analysis Tool

The T-Scope™ tool may be configured for cross-examining gene expression signatures of a given sample with a database of non-hematopoietic cell types (e.g., as described by Hubbard et al., “Analysis of Gene Expression from Systemic Lupus Erythematosus Synovium Reveals Unique Pathogenic Mechanisms [Abstract], Annual Meeting of the American College of Rheumatology; June 2019; Chicago, IL, which is incorporated herein by reference in its entirety). T-Scope™ may comprise a database of 704 transcripts allocated to 45 independent categories. Transcripts detected in the sample are matched to one of the cellular categories within the T-Scope™ tool to derive further insights on tissue cell activity. T-Scope™ may be used downstream of the BIG-CR (Biologically Informed Gene-Clustering) tool to understand which tissue cell types are present. In conjunction with I-Scope™ (which provides information related to immune cells), T-Scope™ may be performed to provide a complete view of all possible cell activity in a given sample.

T-Scope™ addresses the need to understand the involvement of specific tissue cells for a given disease state. While it is helpful to understand the relative up-regulation and down-regulation at the gene expression level, it is even more informative to understand specifically in which cells this is occurring. T-Scope™ may be configured by downloading a set of approximately 10,000 tissue enriched and 8,000 cell line enriched genes from the Human Protein Atlas along with their tissue or cell line designation. Genes differentially expressed in hematopoietic cell datasets are removed and kidney specific genes are added from the GEO repository. T-Scope™ may function by restricting the analysis to genes of known tissue cell heritage and allow for cross-checking against purified single-cell experiments or datasets. The cross-check confirms and categorizes specific transcript signatures to the 45 tissue cell sub-categories, ultimately allowing for cellular activity analysis across multiple samples and disease states. When combined with BIG-CR categories, the cellular activity may be correlated to specific functions within a given tissue cell type.

A sample T-Scope™ workflow may comprise the following steps. First, candidate genes are identified from SLE (systemic lupus erythematosus) differential expression datasets potentially associated with tissue cell expression. Second, using publicly available databases, expression signatures associated with potential tissue cell activity are identified. Third, signatures are cross-referenced with microarray, scRNAseq or RNAseq experiments. Fourth, transcripts are categorized into 45 tissue cell sub-categories and cellular expression is assessed across different samples and disease states. Results may be obtained using T-Scope™ in combination with I-Scope™ for identification of cells post-DE-analysis.

CellScan big data analysis tool

A cloud-based genomic platform may be configured to provide users with access to CellScan™, which comprises a suite of tools for the identification, analysis, and prioritization of targets for drug development and/or repositioning. This platform is powered by a database containing the genomic information gathered from 5000+ autoimmune patients. The cloud-based genomic platform may leverage results from RNAseq and microarray experiments in conjunction with clinical information, such as medication and lab tests, to provide undiscovered insights.

CellScan™ may go beyond typical ‘omics analysis by performing one or more of the following: functionally categorizing genes and their products (e.g., using BIG-CR); deconvolving gene expression data to identify unique immunological cell types from blood or biopsy samples (e.g., using I-Scope™); identifying tissue specific cell from biopsy samples (e.g., using T-Scope™); identifying receptor-ligand interactions and subsequent signaling pathways (e.g., using MS-Scoring™); ranking genes and their products for targeting by drugs and miRNA mimetics (e.g., using Target-Scoring™); and prioritizing FDA-approved drugs and drugs-in-development for treatment in patients or pre-clinical models (e.g., using CoLTs®).

CellScan™ applications may include one or more of: Biomarker Discovery, Disease Mechanisms, Drug Mechanism of Action, Drug Mechanism of Toxicity, and Target Identification and Validation. Experimental approaches supported by CellScan™ may include one or more of: lncRNA, Metabolomics, MicroArray, miRNA, mRNA, qPCR, Proteomics, and RNAseq.

Data analysis and interpretation with CellScan™ may build on comprehensive, manually curated content of a knowledge base. Powerful, quick, and efficient tools may be used to perform deep analysis of NGS and miRNA data to identify gene function, immunological and tissue cell type, pathways, and target/drug appropriate for a specific disease state.

CellScan™ features may be configured to optimize or maximize the impact of information that surfaces in an analysis so that interpretation of a dataset is comprehensive and elucidates actionable insights. These features may include one or more of: NGS RNAseq data analysis, biomarker scoring, and prioritizing targets and drugs for human clinical trials and/or pre-clinical models. The NGS RNAseq data analysis may comprise interrogating RNA and miRNA data for function, cell-type (immunological or tissue) and pathways. The biomarker scoring may comprise using a knowledge base and gene expression data to assess and prioritize biomarkers associated with a target disease or phenotype. The target/drug prioritization may comprise leveraging objective scoring of targets and drugs based on parameters such as scientific rationale, evidence in mouse/human cells, prior clinical data, overall drug properties, and the risk of adverse events.

The knowledge base may be a repository created from millions of individual pieces of information gathered about genes, cells, tissues, drugs, and diseases, and manually reviewed for accuracy and includes rich contextual details and links to original publications. The knowledge base may enable access to relevant and substantiated knowledge from primary literature as well as public and private databases for comprehensive interpretation of NGS/RNAseq data elucidating function/pathways and prioritize targets/drugs for given disease states. An example list of reference databases for the content in CellScan™, with both human and mouse species-specific identifiers supported.

### Ms (Molecular Signature) Scoring™ Analysis Tool

MS-Scoring™ may be configured to identify receptor-ligand interactions and predict ongoing signaling pathways. In addition, MS-Scoring™ may be used to validate molecular pathways as potential targets for new or repurposed drug therapies. The specificity of next-generation drug therapies requires a way to understand the potential of a given therapy to act on the intended biochemical target. Moreover, a potential application of this is the repositioning of drug therapies that may have the correct biochemical targeting to address multiple clinical needs beyond the initial intended therapeutic value.

MS-Scoring™ may be specifically developed to address gaps in the QIAGEN IPA® (Ingenuity Pathway Analysis) tool that does not contain many immunologically relevant pathways. Similar to IPAR, MS-Scoring™ 1 may use log-fold change information to score the target and its signaling pathway to verify the viability of the targets. If the fold-change of the genes of a signaling pathway appears to be upregulated or inhibitors appear to be downregulated, MS-Scoring™ 1 may provide a score of +1. Conversely if the genes of a signaling pathway appear downregulated or the inhibitors upregulated, MS-Scoring™ 1 may provide a score of −1. A score of zero may be provided if no fold-change is observed. The scores may then be summed and normalized across the entire pathway to yield a final % score between-100 (inhibition) and +100 (up-regulation). Higher absolute magnitude scores, scores that are close to −100 or +100, may indicate a high potential for therapeutic targeting. The Fischer's exact test may be performed to determine if there is sufficient overlap of genes between the experimental differentially expressed genes and the genes in the signaling pathway.

A sample MS-Scoring™ 1 workflow may comprise the following steps. First, potential drugs and pathways are identified by LINCS (Library of Integrated Network-Based Cellular Signatures) as candidates for therapeutic intervention. Second, MS-Scoring™ 1 is used to evaluate individual transcript elements of the target pathway. Third, signatures are cross-referenced with purified single-cell microarray datasets and RNAseq experiments. Fourth, scores are compiled and normalized to provide an overall % score for the pathway and higher absolute magnitude scores indicate a higher potential for therapeutic targeting.

MS-Scoring™ 1 may be performed of IL-12 and IL-23 related pathways for targeting using ustekinumab for SLE (systemic lupus erythematosus) drug repositioning (e.g., as described by Grammer et al., 2016, “Drug repositioning in SLE: crowd-sourcing, literature-mining and Big Data analysis,” Lupus, 25 (10), 1150-1170, which is incorporated herein by reference in its entirety).

MS-Scoring™ 2 may utilize custom-defined gene modules that represent a signaling pathway or process and is particularly useful for gene expression datasets from microarray or RNAseq. The MS-Scoring™ 2 tool may be configured to take a deeper look at signaling pathways analyzed using the MS-Scoring™ 1. The tool may analyze raw gene expression data and assess enrichment by the Gene Set Variation Analysis (as described herein), which assigns an indexed score to the individual co-expressed pathways between-1 and +1 indicating levels of down-regulation and up-regulation respectively.

A sample MS-Scoring™ 2 workflow may comprise the following steps. First, a signaling pathway of interest is selected from the MS-Scoring™ 2 menu. Second, a raw gene expression data is inputted into the MS-Scoring™ 2 tool. Third, enrichment of signaling pathway(s) is assessed on a patient by patient basis. Fourth, the data may then be used to drive insight for the target signaling pathways in individual patient samples.

Results from GSVA Analysis on SLE (systemic lupus erythematosus) signaling pathways may be, e.g., as described by Hänzelmann et al., “GSVA: Gene Set Variation Analysis for Microarray and RNA-Seq Data,” BMC Bioinformatics, vol. 14, no. 1, 2013, p. 7., which is incorporated herein by reference in its entirety.

### Colts® (Combined Lupus Treatment Scoring) Analysis Tool

A scoring method called CoLTs®, or Combined Lupus Treatment Scoring, may be configured to assessing and prioritizing the repositioning potential of drug therapies. CoLTs® may rank identified drugs/therapies by a number of essential characteristics, including scientific rationale, experience in lupus mice/human cells (preclinical), previous clinical experience in autoimmunity, drug properties, and safety profile, including adverse events. Face and test validities may be established by scoring standard of care (SOC) medications and confirming the scores with a panel of lupus clinicians. The final result may be the CoLTs® score. A CoLTs® algorithm may also be configured for drugs in development (DID) since they typically do not have drug metabolism and adverse event information available.

CoLTs® may be configured to perform objective scoring of drug molecules based on a hypothesis-based literature search of publicly available databases. The tool has the ability to rank drug molecules from both FDA-approved and non-approved classes and ranked based upon parameters such as scientific rationale, evidence in mouse/human cells, prior clinical data, overall drug properties, and the risk of adverse events. The parameters are used within five independent drug therapy categories: small molecules, biologics, complementary and alternative therapies, and drugs in development.

CoLTs® may address the need for a systematic and objective way to evaluate the potential of drug therapies to be repositioned for treatment of autoimmune diseases, initially within SLE (systemic lupus erythematosus). The composite score may embody all the accessible information in literature databases, inclusive of efficacy and adverse reactions, to be able to assist in the prioritization of drug development. While the composite score takes into account many aspects of a drug, it may heavily weigh the risk of adverse events and ranges from −16 to +11. CoLT Scoring® may be validated through repeated scoring of 215 potential therapies using a total of over 5000 reference data points as well as by clinicians specializing in the field of rheumatology. Specifically, CoLTs®' prediction of Stelara/Ustekinumab to be a top priority biologic for lupus drug repositioning is validated by a successful Phase 2 clinical trial (e.g., as described by Vollenhoven et al., “Efficacy and Safety of Ustekinumab, an IL-12 and IL-23 Inhibitor, in Patients with Active Systemic Lupus Erythematosus: Results of a Multicentre, Double-Blind, Phase 2, Randomised, Controlled Study.” The Lancet, vol. 392, no. 10155, 2018, pp. 1330-1339, which is incorporated herein by reference in its entirety). CoLTs® may be calibrated on SoC (Standard of Care) therapies for the individual autoimmune disease being assessed.

Within the ten major categories, rationale ranges from 0 to +3, mouse/human in vitro experience ranges from −1 to +1, clinical properties are on a scale of −3 to +3, the adverse effect of inducing lupus ranges from −1 to 0, metabolic properties range from −2 to 0, and finally adverse events (such as toxicity, infection, carcinogenic, etc.) were given a score of −5 to 0 (e.g., as described by Grammer et al., 2016, “Drug repositioning in SLE: crowd-sourcing, literature-mining and Big Data analysis,” Lupus, 25 (10), 1150-1170, which is incorporated herein by reference in its entirety). For example, CoLT Scoring® of SOC Therapies in Lupus (Belimumab, HCQ, and Rituximab) may be performed.

### Target Scoring Analysis Tool

The Target scoring algorithm may be configured to prioritize a specific gene or protein that would potentially be a good choice to target with a drug in lupus patients. It may be utilized even if there is currently no drug available to the target gene or protein. The algorithm may be based on the addition of 18 data based determinations plus the overall scientific rationale and generates scores from −13 (not a good target in SLE) to 27 (very promising target in SLE).

Target-Scoring™ may be configured to assessing and prioritizing the potential of molecular targets for further development of drug therapies. The Target-Scoring™ tool is very similar to CoLTs® except it approaches the need for new SLE therapies from a different angle. Target Scoring may be configured to perform an objective assessment of molecular targets for the development of new or repurposed drug therapies. Like CoLTs®, it also derives data from a hypothesis-based literature search and generates a composite score based on the publicly available information. Leveraging the composite score, researchers may better prioritize the development of novel drug therapies addressing the assessed targets of interest.

Target-Scoring™ may utilize 19 different scoring categories to derive a composite score that ranges from −13 to +27 for the suitability of a gene target for SLE therapy development. Target-Scoring™ may be validated through repeated scoring of potential therapies as well as by clinicians (e.g., clinicians specializing in the field of immunology).

### Classifiers

In some embodiments, the present disclosure provides a system, method, or kit having data analysis realized in software application, computing hardware, or both. In various embodiments, the analysis application or system includes at least a data receiving module, a data pre-processing module, a data analysis module, a data interpretation module, or a data visualization module. In one embodiment, the data receiving module may comprise computer systems that connect laboratory hardware or instrumentation with computer systems that process laboratory data. In one embodiment, the data pre-processing module may comprise hardware systems or computer software that performs operations on the data in preparation for analysis. Examples of operations that may be applied to the data in the pre-processing module include affine transformations, denoising operations, data cleaning, reformatting, or subsampling. A data analysis module, which may be specialized for analyzing genomic data from one or more genomic materials, can, for example, take assembled genomic sequences and perform probabilistic and statistical analysis to identify abnormal patterns related to a disease, pathology, state, risk, condition, or phenotype. A data interpretation module may use analysis methods, for example, drawn from statistics, mathematics, or biology, to support understanding of the relation between the identified abnormal patterns and health conditions, functional states, prognoses, or risks. A data visualization module may use methods of mathematical modeling, computer graphics, or rendering to create visual representations of data that may facilitate the understanding or interpretation of results.

Feature sets may be generated from datasets obtained using one or more assays of a biological sample obtained or derived from a subject, and a trained algorithm may be used to process one or more of the feature sets to identify or assess a condition (e.g., a disease or disorder, such as first, second, and/or third disease condition) of a subject. For example, the trained algorithm may be used to apply a machine learning classifier to a plurality of condition-associated genomic loci that are associated with two or more classes of individuals inputted into a machine learning model, in order to classify a subject into one of the two or more classes of individuals. For example, the trained algorithm may be used to apply a machine learning classifier to a plurality of condition-associated that are associated with individuals with known conditions (e.g., a disease or disorder, such as first, second, and/or third disease condition) and individuals not having the condition (e.g., healthy individuals, or individuals who do not have first, second, and/or third disease condition), in order to classify a subject as having the condition (e.g., positive test outcome) or not having the condition (e.g., negative test outcome).

The trained algorithm may be configured to identify the presence (e.g., positive test result) or absence (e.g., negative test result) of one or more conditions (e.g., a disease or disorder, such as first, second, and/or third disease condition) with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%. This accuracy may be achieved for a set of at least about 25, at least about 50, at least about 100, at least about 150, at least about 200, at least about 250, at least about 300, at least about 350, at least about 400, at least about 450, at least about 500, at least about 1,000, or more than about 1,000 independent samples.

The trained algorithm may comprise a machine learning algorithm, such as a supervised machine learning algorithm. The supervised machine learning algorithm may comprise, for example, a Random Forest, a support vector machine (SVM), a neural network, or a deep learning algorithm. The trained algorithm may comprise a classification and regression tree (CART) algorithm. The trained algorithm may comprise an unsupervised machine learning algorithm.

The trained algorithm may comprise a classifier configured to accept as input a plurality of input variables or features (e.g., condition-associated genomic loci) and to produce or output one or more output values based on the plurality of input variables or features (e.g., condition-associated genomic loci). The plurality of input variables or features may comprise one or more datasets indicative of the presence (e.g., positive test result) or absence (e.g., negative test result) of one or more conditions (e.g., a disease or disorder, such as first, second, and/or third disease condition). For example, an input variable or feature may comprise a number of sequences corresponding to or aligning to each of the plurality of condition-associated genomic loci.

The plurality of input variables or features may also include clinical information of a subject, such as health data. For example, the health data of a subject may comprise one or more of: a diagnosis of one or more conditions (e.g., a disease or disorder, such as first, second, and/or third disease condition), a prognosis of one or more conditions (e.g., a disease or disorder, such as first, second, and/or third disease condition), a risk of having one or more conditions (e.g., a disease or disorder, such as first, second, and/or third disease condition), a treatment history of one or more conditions (e.g., a disease or disorder, such as first, second, and/or third disease condition), a history of previous treatment for one or more conditions (e.g., a disease or disorder, such as first, second, and/or third disease condition), a history of prescribed medications, a history of prescribed medical devices, age, height, weight, sex, smoking status, and one or more symptoms of the subject.

For example, the disease or disorder may comprise one or more of: lupus, coronary artery disease (CAD), myocardial infraction, ischemic stroke, coronary atherosclerosis, cardiomyopathy, depression, asthma, chronic obstructive pulmonary disease (COPD), diabetes mellitus, nonalcoholic fatty liver disease, metabolic disorder inflammatory bowel disease, or glomerulonephritis. As another example, the symptoms may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. As another example, the prescribed medications or drugs may include one or more of: antimalarials, corticosteroids, immunosuppressants, and nonsteroidal anti-inflammatory drugs (NSAIDs).

The trained algorithm may comprise a classifier, such that each of the one or more output values comprises one of a fixed number of possible values (e.g., a linear classifier, a logistic regression classifier, etc.) indicating a classification of the sample by the classifier. The trained algorithm may comprise a binary classifier, such that each of the one or more output values comprises one of two values (e.g., {0, 1}, {positive, negative}, or {high-risk, low-risk}) indicating a classification of the sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the sample by the classifier.

The classifier may be configured to classify samples by assigning output values, which may comprise descriptive labels, numerical values, or a combination thereof. Some of the output values may comprise descriptive labels. Such descriptive labels may provide an identification or indication of the presence (e.g., positive test result) or absence (e.g., negative test result) of one or more conditions (e.g., a disease or disorder, such as first, second, and/or third disease condition) of the subject, and may comprise, for example, positive, negative, high-risk, intermediate-risk, low-risk, or indeterminate. Such descriptive labels may provide an identification of a treatment for the one or more conditions of the subject, and may comprise, for example, a therapeutic intervention, a duration of the therapeutic intervention, and/or a dosage of the therapeutic intervention suitable to treat the one or more conditions of the subject. Such descriptive labels may provide an identification of secondary clinical tests that may be appropriate to perform on the subject, and may comprise, for example, an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof. For example, such descriptive labels may provide a prognosis of the one or more conditions of the subject. As another example, such descriptive labels may provide a relative assessment of the one or more conditions of the subject. Some descriptive labels may be mapped to numerical values, for example, by mapping “positive” to 1 and “negative” to 0.

The classifier may be configured to classify samples by assigning output values that comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the one or more conditions (e.g., a disease or disorder, such as first, second, and/or third disease condition) of the subject. Some numerical values may be mapped to descriptive labels, for example, by mapping 1 to “positive” and 0 to “negative.”

The classifier may be configured to classify samples by assigning output values based on one or more cutoff values. For example, a binary classification of samples may assign an output value of “positive” or 1 if the sample indicates that the subject has at least a 50% probability of having one or more conditions (e.g., a disease or disorder, such as first, second, and/or third disease condition), thereby assigning the subject to a class of individuals receiving a positive test result. As another example, a binary classification of samples may assign an output value of “negative” or 0 if the sample indicates that the subject has less than a 50% probability of having one or more conditions (e.g., a disease or disorder), thereby assigning the subject to a class of individuals receiving a negative test result. In this case, a single cutoff value of 50% is used to classify samples into one of the two possible binary output values or classes of individuals (e.g., those receiving a positive test result and those receiving a negative test result). Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, the classifier may be configured to classify samples by assigning an output value of “positive” or 1 if the sample indicates that the subject has a probability of having one or more conditions (e.g., a disease or disorder, such as first, second, and/or third disease condition) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having one or more conditions (e.g., a disease or disorder, such as first, second, and/or third disease condition) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classifier may be configured to classify samples by assigning an output value of “negative” or 0 if the sample indicates that the subject has a probability of having one or more conditions (e.g., a disease or disorder, such as first, second, and/or third disease condition) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having one or more conditions (e.g., a disease or disorder, such as first, second, and/or third disease condition) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classifier may be configured to classify samples by assigning an output value of “indeterminate” or 2 if the sample is not classified as “positive”, “negative”, 1, or 0. In this case, a set of two cutoff values is used to classify samples into one of the three possible output values or classes of individuals (e.g., corresponding to outcome groups of individuals having “low risk,” “intermediate risk,” and “high risk” of having one or more conditions, such as a disease or disorder). Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values or classes of individuals, where n is any positive integer.

The trained algorithm may be trained with a plurality of independent training samples. Each of the independent training samples may comprise a sample from a subject, associated datasets obtained by assaying the sample (as described elsewhere herein), and one or more known output values or classes of individuals corresponding to the sample (e.g., a clinical diagnosis, prognosis, absence, or treatment efficacy of a condition of the subject). Independent training samples may comprise samples and associated datasets and outputs obtained or derived from a plurality of different subjects. Independent training samples may comprise samples and associated datasets and outputs obtained at a plurality of different time points from the same subject (e.g., on a regular basis such as weekly, biweekly, or monthly), as part of a longitudinal monitoring of a subject before, during, and after a course of treatment for one or more conditions of the subject. Independent training samples may be associated with presence of the condition (e.g., training samples comprising samples and associated datasets and outputs obtained or derived from a plurality of subjects known to have the condition). Independent training samples may be associated with absence of the condition (e.g., training samples comprising samples and associated datasets and outputs obtained or derived from a plurality of subjects who are known to not have a previous diagnosis of the condition or who have received a negative test result for the condition).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise samples associated with presence of the condition and/or samples associated with absence of the condition. The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with presence of the condition (e.g., a disease or disorder, such as first, second, and/or third disease condition). The trained algorithm may be trained with no more than about 500, no more than about 450, no more than about 400, no more than about 350, no more than about 300, no more than about 250, no more than about 200, no more than about 150, no more than about 100, or no more than about 50 independent training samples associated with absence of the condition (e.g., a disease or disorder, such as first, second, and/or third disease condition). In some embodiments, the sample is independent of samples used to train the trained algorithm.

The trained algorithm may be trained with a first number of independent training samples associated with a presence of the condition (e.g., a disease or disorder, such as first, second, and/or third disease condition) and a second number of independent training samples associated with an absence of the condition (e.g., a disease or disorder, such as first, second, and/or third disease condition). The first number of independent training samples associated with presence of the condition (e.g., a disease or disorder, such as first, second, and/or third disease condition) may be no more than the second number of independent training samples associated with absence of the condition (e.g., a disease or disorder, such as first, second, and/or third disease condition). The first number of independent training samples associated with a presence of the condition (e.g., a disease or disorder) may be equal to the second number of independent training samples associated with an absence of the condition (e.g., a disease or disorder, such as first, second, and/or third disease condition). The first number of independent training samples associated with a presence of the condition (e.g., a disease or disorder, such as first, second, and/or third disease condition) may be greater than the second number of independent training samples associated with an absence of the condition (e.g., a disease or disorder, such as first, second, and/or third disease condition).

The trained algorithm may comprise a classifier configured to identify the presence (e.g., positive test result) or absence (e.g., negative test result) of one or more conditions (e.g., a disease or disorder, such as first, second, and/or third disease condition) at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of identifying the presence (e.g., positive test result) or absence (e.g., negative test result) of the one or more conditions by the trained algorithm may be calculated as the percentage of independent test samples (e.g., subjects known to have the condition or subjects with negative clinical test results for the condition) that are correctly identified or classified as having or not having the condition.

The trained algorithm may comprise a classifier configured to identify one or more conditions (e.g., a disease or disorder, such as first, second, and/or third disease condition) with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the condition using the trained algorithm may be calculated as the percentage of samples identified or classified as having the condition that correspond to subjects that truly have the condition.

The trained algorithm may comprise a classifier configured to identify one or more conditions (e.g., a disease or disorder, such as first, second, and/or third disease condition) with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the condition using the trained algorithm may be calculated as the percentage of samples identified or classified as not having the condition that correspond to subjects that truly do not have the condition.

The trained algorithm may comprise a classifier configured to identify one or more conditions (e.g., a disease or disorder, such as first, second, and/or third disease condition) with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the condition using the trained algorithm may be calculated as the percentage of independent test samples associated with presence of the condition (e.g., subjects known to have the condition) that are correctly identified or classified as having the condition.

The trained algorithm may comprise a classifier configured to identify one or more conditions (e.g., a disease or disorder, such as first, second, and/or third disease condition) with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the condition using the trained algorithm may be calculated as the percentage of independent test samples associated with absence of the condition (e.g., subjects with negative clinical test results for the condition) that are correctly identified or classified as not having the condition.

The trained algorithm may comprise a classifier configured to identify the presence (e.g., positive test result) or absence (e.g., negative test result) of one or more conditions (e.g., a disease or disorder, such as first, second, and/or third disease condition) with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the Receiver Operator Characteristic (ROC) curve (e.g., the area under the ROC curve) associated with the trained algorithm in classifying samples as having or not having the condition.

Classifiers of the trained algorithm may be adjusted or tuned to improve or optimize one or more performance metrics, such as accuracy, PPV, NPV, clinical sensitivity, clinical specificity, AUC, or a combination thereof (e.g., a performance index incorporating a plurality of such performance metrics, such as by calculating a weight sum therefrom), of identifying the presence (e.g., positive test result) or absence (e.g., negative test result) of the condition. The classifiers may be adjusted or tuned by adjusting parameters of the classifiers (e.g., a set of cutoff values used to classify a sample as described elsewhere herein, or weights of a neural network) to improve or optimize the performance metrics. The one or more classifiers may be adjusted or tuned so as to reduce an overall classification error (e.g., an “out-of-bag” or oob error rate for a Random Forest classifier). The one or more classifiers may be adjusted or tuned continuously during the training process (e.g., as sample datasets are added to the training set) or after the training process has completed.

The trained algorithm may comprise a plurality of classifiers (e.g., an ensemble) such that the plurality of classifications or outcome values of the plurality of classifiers may be combined to produce a single classification or outcome value for the sample. For example, a sum or a weighted sum of the plurality of classifications or outcome values of the plurality of classifiers may be calculated to produce a single classification or outcome value for the sample. As another example, a majority vote of the plurality of classifications or outcome values of the plurality of classifiers may be identified to produce a single classification or outcome value for the sample. In this manner, a single classification or outcome value may be produced for the sample having greater confidence or statistical significance than the individual classifications or outcome values produced by each of the plurality of classifiers.

After the trained algorithm is initially trained, a subset of the inputs may be identified as most influential or most important to be included for making high-quality classifications (e.g., having highest permutation feature importance). For example, a subset of the panel of condition-associated genomic loci may be identified as most influential or most important to be included for making high-quality classifications or identifications of conditions (or sub-types of conditions). The panel of condition-associated genomic loci, or a subset thereof, may be ranked based on classification metrics indicative of each influence or importance of each individual condition-associated genomic locus toward making high-quality classifications or identifications of conditions (or sub-types of conditions). Such metrics may be used to reduce, in some cases significantly, the number of input variables (e.g., predictor variables) that may be used to train the one or more classifiers of the trained algorithm to a desired performance level (e.g., based on a desired minimum accuracy, PPV, NPV, clinical sensitivity, clinical specificity, AUC, or a combination thereof).

For example, if training a classifier of the trained algorithm with a plurality comprising several dozen or hundreds of input variables to the classifier results in an accuracy of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

As another example, if training a classifier of the trained algorithm with a plurality comprising several dozen or hundreds of input variables to the classifier results in a sensitivity or specificity of classification of more than 99%, then training the classifier of the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality may yield decreased but still acceptable sensitivity or specificity of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%).

The subset of the plurality of input variables (e.g., the panel of condition-associated genomic loci) to the classifier of the trained algorithm may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics (e.g., permutation feature importance).

Upon identifying the subject as having one or more conditions (e.g., a disease or disorder, such as first, second, and/or third disease condition), the subject may be optionally provided with a therapeutic intervention (e.g., prescribing an appropriate course of treatment to treat the one or more conditions of the subject). The therapeutic intervention may comprise a prescription of an effective dose of a drug, a further testing or evaluation of the condition, a further monitoring of the condition, or a combination thereof. If the subject is currently being treated for the condition with a course of treatment, the therapeutic intervention may comprise a subsequent different course of treatment (e.g., to increase treatment efficacy due to non-efficacy of the current course of treatment).

The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

The therapeutic intervention may comprise recommending the subject for a secondary clinical test to confirm a diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

The feature sets (e.g., comprising quantitative measures of a panel of condition-associated genomic loci) may be analyzed and assessed (e.g., using a trained algorithm comprising one or more classifiers) over a duration of time to monitor a patient (e.g., subject who has a condition or who is being treated for a condition). In such cases, the feature sets of the patient may change during the course of treatment. For example, the quantitative measures of the feature sets of a patient with decreasing risk of the condition due to an effective treatment may shift toward the profile or distribution of a healthy subject (e.g., a subject without the condition). Conversely, for example, the quantitative measures of the feature sets of a patient with increasing risk of the condition due to an ineffective treatment may shift toward the profile or distribution of a subject with higher risk of the condition or a more advanced stage or severity of the condition.

The condition of the subject may be monitored by monitoring a course of treatment for treating the condition of the subject. The monitoring may comprise assessing the condition of the subject at two or more time points. The assessing may be based at least on the feature sets (e.g., quantitative measures of a panel of condition-associated genomic loci) determined at each of the two or more time points. The therapeutic intervention may include prescribed medications or drugs, which may include one or more of: antimalarials, corticosteroids, immunosuppressants, and nonsteroidal anti-inflammatory drugs (NSAIDs). The therapeutic intervention may be effective to alleviate or decrease one or more symptoms, which may include one or more of: alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof. The assessing may be based at least on the presence, absence, or severity of one or more symptoms, such as alopecia, anti-dsDNA seropositivity, arthritis, fever, hematuria, leukopenia, low serum complement, mucosal ulcer, myositis, pericarditis, pleurisy, proteinuria, pyuria, rash, thrombocytopenia, urinary cast, vasculitis, visual disturbance, or a combination thereof.

In some embodiments, a difference in the feature sets (e.g., quantitative measures of a panel of condition-associated genomic loci) determined between the two or more time points may be indicative of one or more clinical indications, such as (i) a diagnosis of the condition of the subject, (ii) a prognosis of the condition of the subject, (iii) an increased risk of the condition of the subject, (iv) a decreased risk of the condition of the subject, (v) an efficacy of the course of treatment for treating the condition of the subject, and (vi) a non-efficacy of the course of treatment for treating the condition of the subject.

In some embodiments, a difference in the feature sets (e.g., quantitative measures of a panel of condition-associated genomic loci) determined between the two or more time points may be indicative of a diagnosis of the condition of the subject. For example, if the condition was not detected in the subject at an earlier time point but was detected in the subject at a later time point, then the difference is indicative of a diagnosis of the condition of the subject. A clinical action or decision may be made based on this indication of diagnosis of the condition of the subject, such as, for example, prescribing a new therapeutic intervention for the subject. The clinical action or decision may comprise recommending the subject for a secondary clinical test to confirm the diagnosis of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a prognosis of the condition of the subject.

In some embodiments, a difference in the feature sets (e.g., quantitative measures of a panel of condition-associated genomic loci) determined between the two or more time points may be indicative of the subject having an increased risk of the condition. For example, if the condition was detected in the subject both at an earlier time point and at a later time point, and if the difference is a negative difference (e.g., the quantitative measures of a panel of condition-associated genomic loci increased from the earlier time point to the later time point), then the difference may be indicative of the subject having an increased risk of the condition. A clinical action or decision may be made based on this indication of the increased risk of the condition, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of the subject having a decreased risk of the condition. For example, if the condition was detected in the subject both at an earlier time point and at a later time point, and if the difference is a positive difference (e.g., the quantitative measures of a panel of condition-associated genomic loci decreased from the earlier time point to the later time point), then the difference may be indicative of the subject having a decreased risk of the condition. A clinical action or decision may be made based on this indication of the decreased risk of the condition (e.g., continuing or ending a current therapeutic intervention) for the subject. The clinical action or decision may comprise recommending the subject for a secondary clinical test to confirm the decreased risk of the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of an efficacy of the course of treatment for treating the condition of the subject. For example, if the condition was detected in the subject at an earlier time point but was not detected in the subject at a later time point, then the difference may be indicative of an efficacy of the course of treatment for treating the condition of the subject. A clinical action or decision may be made based on this indication of the efficacy of the course of treatment for treating the condition of the subject, e.g., continuing or ending a current therapeutic intervention for the subject. The clinical action or decision may comprise recommending the subject for a secondary clinical test to confirm the efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In some embodiments, a difference in the feature sets (e.g., quantitative measures of a panel of condition-associated genomic loci) determined between the two or more time points may be indicative of a non-efficacy of the course of treatment for treating the condition of the subject. For example, if the condition was detected in the subject both at an earlier time point and at a later time point, and if the difference is a negative or zero difference (e.g., the quantitative measures of a panel of condition-associated genomic loci increased or remained at a constant level from the earlier time point to the later time point), and if an efficacious treatment was indicated at an earlier time point, then the difference may be indicative of a non-efficacy of the course of treatment for treating the condition of the subject. A clinical action or decision may be made based on this indication of the non-efficacy of the course of treatment for treating the condition of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the condition. This secondary clinical test may comprise an imaging test, a blood test, a computed tomography (CT) scan, a magnetic resonance imaging (MRI) scan, an ultrasound scan, a chest X-ray, a positron emission tomography (PET) scan, a PET-CT scan, or any combination thereof.

In various embodiments, machine learning methods are applied to distinguish samples in a population of samples.

### Kits

The present disclosure provides kits for identifying or monitoring a disease or disorder (e.g., first, second, and/or third disease condition) of a subject. A kit may comprise probes for identifying a quantitative measure (e.g., indicative of a presence, absence, or relative amount) of sequences at each of a panel of condition-associated genomic loci in a sample of the subject. A quantitative measure (e.g., indicative of a presence, absence, or relative amount) of sequences at each of a panel of condition-associated genomic loci in the sample may be indicative of the disease or disorder (e.g., first, second, and/or third disease condition) of the subject. The probes may be selective for the sequences at the panel of condition-associated genomic loci in the sample. A kit may comprise instructions for using the probes to process the sample to generate datasets indicative of a quantitative measure (e.g., indicative of a presence, absence, or relative amount) of sequences at each of the panel of condition-associated genomic loci in a sample of the subject.

The probes in the kit may be selective for the sequences at the panel of condition-associated genomic loci in the sample. The probes in the kit may be configured to selectively enrich nucleic acid (e.g., RNA or DNA) molecules corresponding to the panel of condition-associated genomic loci. The probes in the kit may be nucleic acid primers. The probes in the kit may have sequence complementarity with nucleic acid sequences from one or more of the panel of condition-associated genomic loci. The panel of condition-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more distinct condition-associated genomic loci.

The instructions in the kit may comprise instructions to assay the sample using the probes that are selective for the sequences at the panel of condition-associated genomic loci in the cell-free biological sample. These probes may be nucleic acid molecules (e.g., RNA or DNA) having sequence complementarity with nucleic acid sequences (e.g., RNA or DNA) from one or more of the plurality of panel of condition-associated genomic loci. These nucleic acid molecules may be primers or enrichment sequences. The instructions to assay the cell-free biological sample may comprise introductions to perform array hybridization, polymerase chain reaction (PCR), or nucleic acid sequencing (e.g., DNA sequencing or RNA sequencing) to process the sample to generate datasets indicative of a quantitative measure (e.g., indicative of a presence, absence, or relative amount) of sequences at each of the panel of condition-associated genomic loci in the sample. A quantitative measure (e.g., indicative of a presence, absence, or relative amount) of sequences at each of a panel of condition-associated genomic loci in the sample may be indicative of a disease or disorder (e.g., first, second, and/or third disease condition).

The instructions in the kit may comprise instructions to measure and interpret assay readouts, which may be quantified at one or more of the panel of condition-associated genomic loci to generate the datasets indicative of a quantitative measure (e.g., indicative of a presence, absence, or relative amount) of sequences at each of the panel of condition-associated genomic loci in the sample. For example, quantification of array hybridization or polymerase chain reaction (PCR) corresponding to the panel of condition-associated genomic loci may generate the datasets indicative of a quantitative measure (e.g., indicative of a presence, absence, or relative amount) of sequences at each of the panel of condition-associated genomic loci in the sample. Assay readouts may comprise quantitative PCR (qPCR) values, digital PCR (dPCR) values, digital droplet PCR (ddPCR) values, fluorescence values, etc., or normalized values thereof.

In some embodiments, the dataset comprises RNA gene expression or transcriptome data, DNA genomic data, or a combination thereof. In some embodiments, the biological sample is selected from the group consisting of: a whole blood (WB) sample, a PBMC sample, a tissue sample, and a cell sample. In some embodiments, assessing the SLE condition of the subject comprises determining a diagnosis of the SLE condition, a prognosis of the SLE condition, a susceptibility of the SLE condition, a treatment for the SLE condition, or an efficacy or non-efficacy of a treatment for the SLE condition.

In some embodiments, the method further comprises determining a diagnosis of the SLE condition with a sensitivity of at least about 70%. In some embodiments, the method further comprises determining a diagnosis of the SLE condition with a specificity of at least about 70%. In some embodiments, the method further comprises determining a diagnosis of the SLE condition with a positive predictive value of at least about 70%. In some embodiments, the method further comprises determining a diagnosis of the SLE condition with a negative predictive value of at least about 70%. In some embodiments, the method further comprises determining a diagnosis of the SLE condition with an Area Under Curve (AUC) of at least about 70%. In some embodiments, the method further comprises determining a likelihood of the diagnosis of the SLE condition of the subject.

In some embodiments, the method further comprises generating a plurality of drug candidates for the SLE condition of the subject. In some embodiments, the method further comprises evaluating or predicting a relative efficacy of the plurality of drug candidates for the SLE condition of the subject. In some embodiments, the method further comprises providing a therapeutic intervention comprising one or more of the plurality of drug candidates for the SLE condition of the subject.

In some embodiments, the method further comprises monitoring the SLE condition of the subject, wherein the monitoring comprises assessing the SLE condition of the subject at each of a plurality of time points, and processing the plurality of assessments of the SLE condition of the subject at each of the plurality of time points.

### EXAMPLES

The following illustrative examples are representative of embodiments of the software applications, systems, and methods described herein and are not meant to be limiting in any way.

### Example 1: Molecular Pathways Identified from Risk Alleles Demonstrate Mechanistic Differences in Systemic Lupus Erythematosus Patients of East Asian and European Ancestry

Systemic lupus erythematosus (SLE) (OMIM: 152700) is a complex autoimmune disease characterized by clinical and genetic heterogeneity. Individuals of East Asian ancestry (AsA) have a greater prevalence of renal involvement, infections and cardiovascular complications compared to individuals of European ancestry (EA). In particular, lupus nephritis and end stage renal disease (LN/ESRD) are severe complications of SLE that are more prevalent in patients of AsA ancestry than patients of EA ancestry. Whereas some of this variation may be accounted for by confounding environmental and/or socioeconomic factors, it is unclear why AsA ancestry remains associated with clinical severity and sub-phenotypes in SLE. Immunochip-based and genome-wide association (GWA) studies have revealed important ancestry-specific and trans-ancestral risk associations predisposing to SLE. Recent meta-analyses of European and Chinese GWAS data suggest that the greater disease burden evident in East Asian populations is at least partially a consequence of different risk variant frequencies. Although these and other studies enable a better understanding of the genetic architecture of SLE, they tend to focus on only the most significant associations and linked genes, and thereby do not capture the totality of genetic variation. Critically, genetic analyses to date have been unable to provide a clear path toward novel therapeutic development.

### Results:

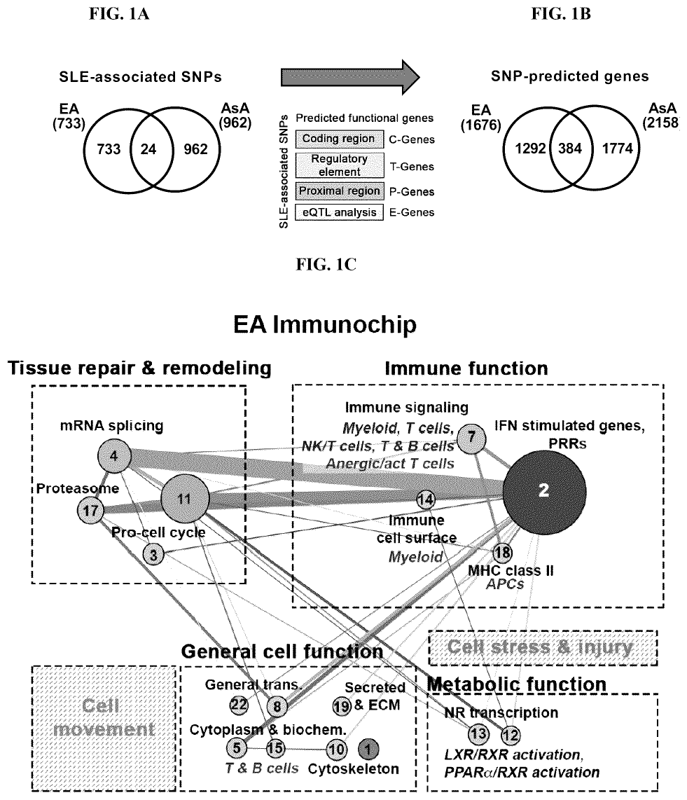

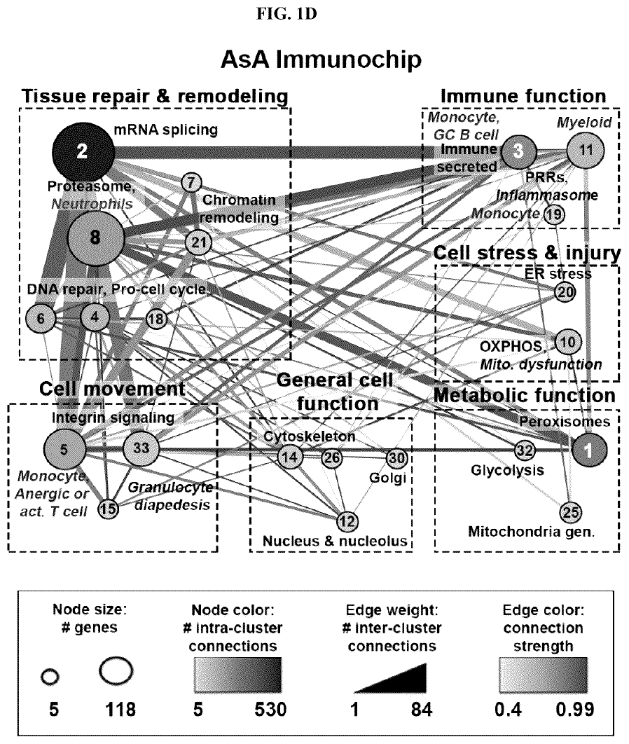

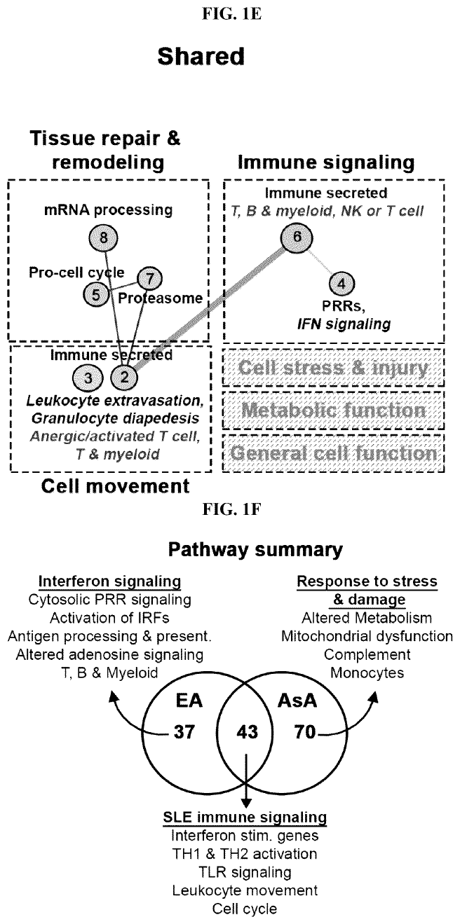

Delineation of signaling pathways identified by ancestry-specific SNP-associated genes. Immunochip-based association analyses identified 986 SNPs reported as significantly associated with SLE in AsA patients and 757 SNPs associated in EA populations (FIG. 1A). A bioinformatics-based approach was used to identify the most plausible genes affected by the SLE-SNP association, revealing 1,676 EA and 2,158 AsA predicted genes (FIG. 1B). EA-associated genes were dominated by the functional category for interferon stimulated genes, along with multiple canonical pathways related to the activation of pattern recognition receptors and downstream type I interferon signaling (FIGS. 1C, F). Pathways associated with SLE in AsA were indicative of a diverse range of biological processes, many related to protein metabolic functions (FIGS. 1D, F). The data sets used are listed in Table 12.

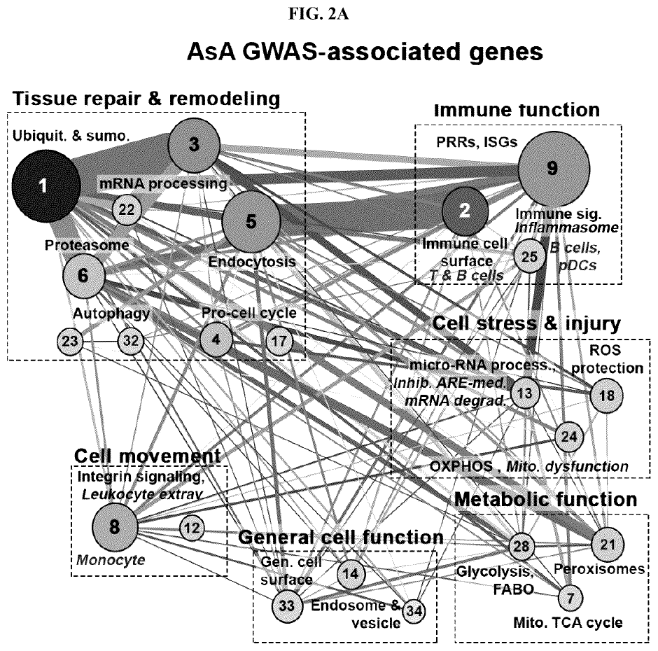

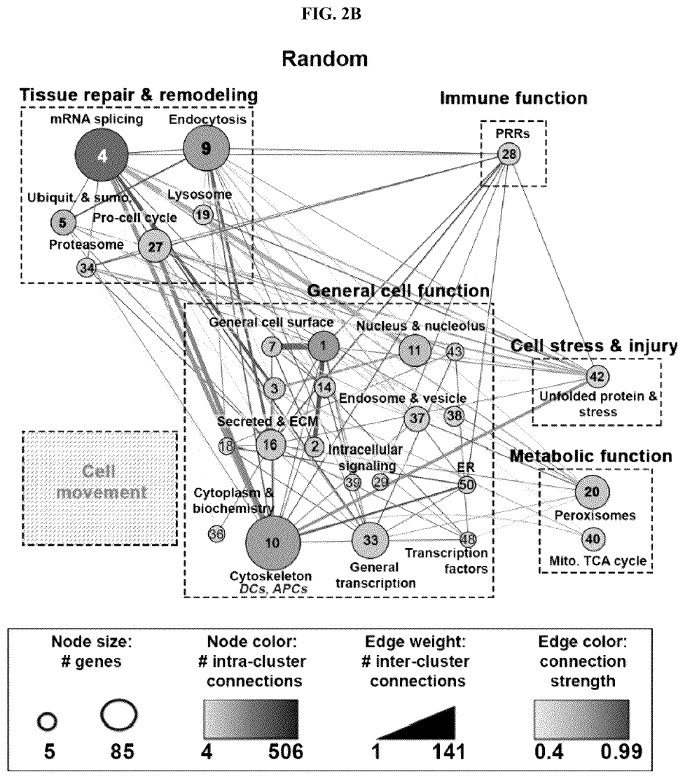

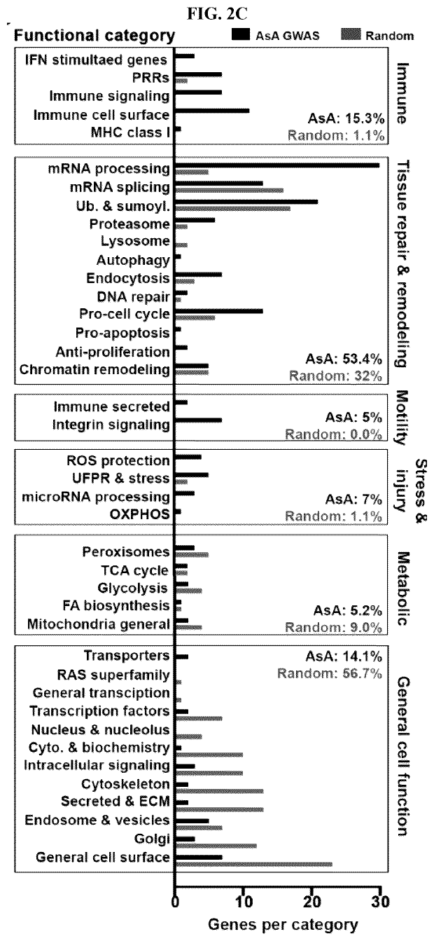

Validation of AsA-enriched molecular pathways using summary GWAS data. Summary data combined from GWAS (Lessard et al., 2016; Morris et al., 2016) identified 1350 SNPs significantly associated with SLE in AsA patients that predicted a validation gene cohort of over 2000 genes used for connectivity mapping. Functional annotation and pathway analysis of each cluster revealed striking similarity to those derived from the AsA Immunochip-associated genes (FIG. 2A). An equivalent cohort of random genes generally formed smaller clusters, exhibited fewer intra- and inter-cluster connections, and were primarily enriched in functional categories mostly related to basic cell function (general cell surface, secreted and ECM) (FIGS. 2B-C).

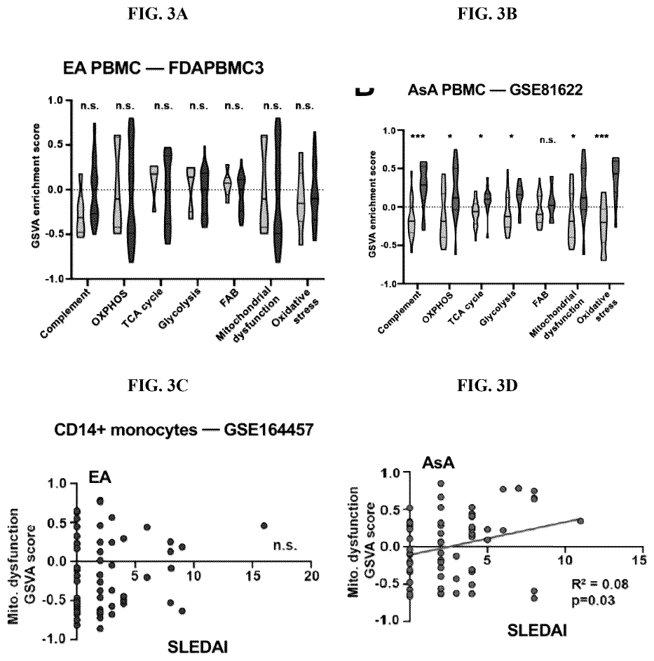

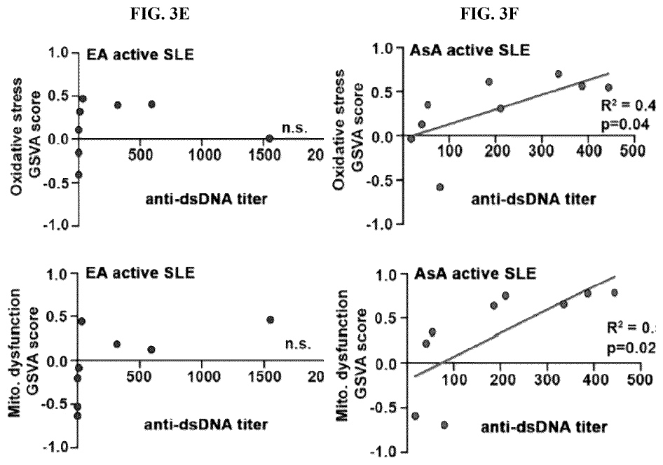

SNP-associated pathways inform gene signatures for GSVA analysis in gene expression datasets. To test the pathway predictions, Gene Set Variation Analysis (GSVA) was applied to determine the relative enrichment of gene signatures identified in peripheral blood mononuclear cell (PBMC) samples from SLE patients (EA and AsA) and controls. Consistent with our predictions, AsA SLE patients exhibited significant enrichment in signatures for mitochondrial dysfunction, oxidative stress metabolic processes (FIGS. 3A-B). The gene signatures are listed in Tables 1 to 11.

Using linear regression, GSVA scores for mitochondrial dysfunction demonstrated a significant positive relationship with SLEDAI in AsA but not EA SLE monocytes (FIGS. 3 C-D). Similarly, enrichment scores for oxidative stress and mitochondrial dysfunction showed a significant positive correlation with anti-dsDNA titers among AsA SLE patients with active disease (SLEDAI≥6) compared to EA patients (FIGS. 3 E-F).

### CONCLUSIONS

FIG. 4 shows the dominant molecular pathways involved in development of lupus in patients of Asian and European Ancestry, and possible treatments associated with the pathways. Genes linked to SNPs in AsA cohorts were enriched in processes related to translation/mRNA processing, metabolism, cell stress and mitochondrial dysfunction. EA tended to include immune processes and IFN signaling.

TABLE 1
Immunochip Cluster 11 (gene symbols)

| | Functional | Predicted |
|---|---|---|
| | annotation | drugs |
| STX3, SHPK, SNRNP35, ZMAT5TXK, PSMG1, | JAK signaling | Baricitinib, |
| WIPF2, MTMR2, MANBA, LIMD1, SV2C, SH2B3, | | carfilzomib, |
| IL18RAP, IL21R, VPS28, PINLYP, GGA2, LAT2, | | curcumol, |
| MTMR3, PRKCI, PTPRK, HCFC1, EFNA5, EEA1 | | decernotinib, |
| GFRA4, TCERG1, PLOD1.LIF, IL4, FHL2, | | delgocitinib, |
| QRICH1, SF3B2, U2AF1L4, CHERP, PGM1, | | ruxolitinib, solicitinib, |
| DNASE1L1, CNN2, PGLYRP1, CAMP, SNRPA1, | | tofacitinib, |
| RUNX3, MYSM1, E2F2, UBLCP1, BAIAP2L1, | | upadacitinib, |
| CDC25A, FUS, ARMC1, NCKIPSD, EDNRB, NMB, | | bortezomib, |
| GPR65, UTS2ARHGEF25 | | densosumab, |
| SYT1, RAB5B, WASL, EPHB2, RAB5C, HIP1R, | | filgotinib, idelalisib, |
| AP4B1, ANAPC16, EHMT2, PSMB9, PSME3, | | KZR-616, peficitinib |
| CBX7, ALDH1A3, CDKN1A, TNFSF11, PIK3CG, | | |
| EGFR, PIK3R2, JAK2, NFKB1, CCNT2, P2RY11 | | |

TABLE 2

| | Functional | |
|---|---|---|
| Immunochip Cluster 10 (gene symbols) | annotation | Predicted drugs |
| PDCD5, NDUFAF2, IDH3B, TMA7, NIT1, VBP1, | Oxidative | Metformin, |
| NDUFA5, NDUFA9, COX6B1, UQCR10, COX17, | phosphorylation | phenformin, BAY84- |
| NDUFAB1, COX6A2, HINT1, UQCRQ | | 2243, CAI, ME344, |
| PSMA6, ATP2A2 | | fenofibrate, |
| | | lonidamine, arsenic |
| | | trioxide, atovaquone, |
| | | hydrocortisone, α- |
| | | TOS, thapsigargin |

TABLE 3
Immunochip Cluster 25 (gene symbols)

| | Functional | Predicted |
|---|---|---|
| | annotation | drugs |
| SLC25A6, TAZ, TIMM17A, CISD2, | Sirtuin signaling | Resveratrol, |
| CISD1, PPIF, VDAC3, C6 | pathway | cyclosporin A |

TABLE 4
Immunochip Cluster 20 (gene symbols)

| | Functional | Predicted |
|---|---|---|
| | annotation | drugs |
| CALR, MSR1, APOM, | Mitochondrial | Resveratrol, N-acetyl |
| VIMP, PEX3, COL4A2 | dysfunction | L-cysteine, SKQ1, |
| | | ubiquinone, mitoVitE, |
| | | mitoTEMPO, vitamin |
| | | E, vitamin C |

TABLE 5
Immunochip Cluster 32 (gene symbols)

| | Functional | Predicted |
|---|---|---|
| | annotation | drugs |
| MOBP, AMT, ALDH9A1, | Glycolysis | Cylcosporin A |
| CNDP1, GAD1, ADSSL1 | | |

TABLE 6
GWAS Cluster 18 (gene symbols)

| | Functional | Predicted |
|---|---|---|
| | annotation | drugs |
| PFKM, PDXK, NEU1, MGST3, GBE1, UGDH, | Reactive oxygen | Resveratrol, N-acetyl |
| ERO1LB, PRDX1, TNFAIP6, ASAH1, GUSB, | species (ROS) | L-cysteine, SKQ1, |
| ARSG, PRDX6, GGH, HYAL4, PYGB, ARSB, | protection | ubiquinone, mitoVitE, |
| TXNDC5, GNS, GPX5, GPX6 | | mitoTEMPO, vitamin |
| | | E, vitamin C, ALT- |
| | | 2074, Ebselen, |
| | | GC4419 |

TABLE 7
GWAS Cluster 1 (gene symbols)

| | Functional | Predicted |
|---|---|---|
| | annotation | drugs |
| ZYG11A, ZYG11B, SERP2, SPCS3, RNF5, RNF169, | MTOR signaling | Sirolimus, |
| RNF11, USP25, ZFAND5, UBQLNL, PPP1R9A, | | everolimus, |
| SPCS2, PARP11, NPAS3, SLIT2, KCNQ5, KCNG3, | | temsirolimus |
| RAD23B, GFM1, SRP54, FRRS1, USP47, | | |
| FAM168A, EPM2A, MRPL13, WNT3A, HBS1L, | | |
| DCAF12, UROD, DCAF6, , RPS27L, RSRC1, | | |
| SKIV2L, RPL9, RPL6, SSR1, DCUN1D3, PPP2CA, | | |
| RPS10, RPS25, RPS18, RPL3, RPL10A, RPS6, RPS5 | | |
| SEC61A1, ARCN1, CERKL, TCEB1, GAN, ASB14, | | |
| ANAPC7, HERC1, UBE2G1, CUL5, HECTD3, | | |
| FBXL22, FBXW5, RNF25, KCTD7, SKP2, CBLB, | | |
| UBE2R2, HACE1, RNF114, SKP1, ANAPC13, RPL8 | | |
| UBA52, PHC3, RPS8 | | |

TABLE 8
GWAS Cluster 6 (gene symbols)

| | Functional | Predicted |
|---|---|---|
| | annotation | drugs |
| USP37, TAP2, TAP1, UBLCP1, JAZF1, SOD1, | JAK signaling | Baricitinib, |
| TNKS, PELP1, STAMBP, CDKL1, NAA10, | | carfilzomib, |
| TUBAL3, TUBA3E, FKBP9, RUVBL1, TTLL3, | | curcumol, |
| MARS, COPRS, DDX39B, CHTOP, BCLAF1, | | decernotinib, |
| HSP90AB1, PSMB11, PSMB8, PSMB9, CCNE2, | | delgocitinib, |
| CCT6A, AHCY, NOTCH4, CCND1, JAK2, PSMB5, | | ruxolitinib, solicitinib, |
| ABCB5 | | tofacitinib, |
| | | upadacitinib, |
| | | bortezomib, |
| | | densosumab, |
| | | filgotinib, idelalisib, |
| | | KZR-616, peficitinib, |
| | | ribociclib, |
| | | palbociclib, sirolimus |

TABLE 9
GWAS Cluster 13 (gene symbols)

| | Functional | Predicted |
|---|---|---|
| | annotation | drugs |
| WWC1, MPP5, LRP1, AGO4, ETS1, ERN1, | microRNA | Cyclosporin A, |
| KIAA0391, AGO3, NOTCH3, AGO1, H2AFX, | processing | thapsigargin |
| HIST1H2BL, HIST1H2BK, ATP2A2, ATF6B, | | |
| HSPA1A, HSPA1B, PPID | | |

TABLE 10
GWAS Cluster 24 (gene) symbols

| | Functional | Predicted |
|---|---|---|
| | annotation | drugs |
| MRPL5, MRPL48, DMXL2, C14orf2, C17orf80, | Mitochondrial | Resveratrol, N-acetyl |
| ATP6V0B, MRPS21, ATP6V1G2, ATP6V1C2, | dysfunction | L-cysteine, SKQ1, |
| CCDC115, COX6B1, ATP5L, ACADVL | | ubiquinone, mitoVitE, |
| | | mitoTEMPO, vitamin |
| | | E, vitamin C |

TABLE 11
GWAS Cluster 21 (gene symbols)

| | Functional | Predicted |
|---|---|---|
| | annotations | drugs |
| TNIP1, LTB, TNF, LTA, TNFAIP3, EPHX2, | TNF signaling | Adalimumab, AMG- |
| MPV17, PEX13, CROT, CDYL2, STAT1, NOS2, | | 811, baricitinib, |
| ASL, TAB1, PSMA6, CALML6, ACSL1, SCP2, | | BMS-986165, |
| ABCD2 | | certolizumab, |
| | | dacomitinib, |
| | | etanercept, filgotinib, |
| | | iguratimod, |
| | | infliximab, |
| | | ruxolitinib, solicitinib, |
| | | tabalumab, |
| | | trofinetide, |
| | | upadacitinib |

TABLE 12
SLE datasets used

| Dataset | Sample | Sex | Ancestry | SLEDAI | SLE | Control |
|---|---|---|---|---|---|---|
| FDAPBMC 3 | PBMC | Female | EA | 0-8 | 12 | 5 |
| GSE81622 | PBMC | Mixed | AsA | Unknown | 12 | 21 |
| GSE164457 | CD14 | Mixed | EA & AsA | 0-16 | 56 EA; | 0 |
| | mono. | | | | 61 AsA | |

### Example 2: Molecular Pathways Identified from Single Nucleotide Polymorphisms Demonstrate Mechanistic Differences in Systemic Lupus Erythematosus Patients of Asian and European Ancestry

Systemic lupus erythematosus (SLE) is a multi-organ autoimmune disorder with a prominent genetic component. Individuals of Asian-Ancestry (AsA) disproportionately experience more severe SLE compared to individuals of European-Ancestry (EA), including increased renal involvement and tissue damage. However, the mechanisms underlying elevated severity in the AsA population remain unclear. Here, we utilized available gene expression data and genotype data based on all non-HLA SNP associations in EA and AsA SLE patients detected using the Immunochip genotyping array. We identified 2778 ancestry-specific and 327 trans-ancestry SLE-risk polymorphisms. Genetic associations were examined using connectivity mapping and gene signatures based on predicted biological pathways and were used to interrogate gene expression datasets. SLE-associated pathways in AsA patients included elevated oxidative stress, altered metabolism and mitochondrial dysfunction, whereas SLE-associated pathways in EA patients included a robust interferon response (type I and II) related to enhanced cytosolic nucleic acid sensing and signaling. An independent dataset derived from summary genome-wide association data in an AsA cohort was interrogated and identified similar molecular pathways. Finally, gene expression data from AsA SLE patients corroborated the molecular pathways predicted by SNP associations. Identifying ancestry-related molecular pathways predicted by genetic SLE risk may help to disentangle the population differences in clinical severity that impact AsA and EA individuals with SLE.

Systemic lupus erythematosus (SLE) (OMIM: 152700) is a complex autoimmune disease characterized by clinical and genetic heterogeneity. Individuals of East Asian ancestry (AsA) have a greater prevalence of renal involvement, infections and cardiovascular complications compared to individuals of European ancestry (EA) (1). In particular, lupus nephritis and end stage renal disease (LN/ESRD) are severe complications of SLE that are more prevalent in patients of AsA ancestry than patients of EA ancestry (2,3,4). Whereas some of this variation may be accounted for by confounding environmental and/or socioeconomic factors (5), it is unclear why AsA ancestry remains associated with clinical severity and sub-phenotypes in SLE.

Immunochip-based and genome-wide association (GWA) studies have revealed important ancestry-specific and trans-ancestral risk associations predisposing to SLE (6,7,8,9,10). Recent meta-analyses of European and Chinese GWAS data suggest that the greater disease burden evident in East Asian populations is at least partially a consequence of different risk variant frequencies (9). Although these and other studies enable a better understanding of the genetic architecture of SLE, they tend to focus on only the most significant associations and linked genes, and thereby do not capture the totality of genetic variation. Critically, genetic analyses to date have been unable to provide a clear path toward novel therapeutic development. This shortcoming is of particular concern with respect to AsA patients, where the control of disease activity remains suboptimal (5). Here, we undertook a bioinformatics-driven approach to identify a comprehensive list of ancestry-specific and shared SLE-associated genes, using eQTL mapping, the identification of functional variants in coding regions and variants impacting transcription factor binding site occupancy, as well as SNP-gene proximity. Together, this approach identified 3105 potential SLE-associated genes in one or more ancestral groups (1349 EA, 1429 AsA and 327 trans-ancestral). Connectivity mapping and network analysis were used to identify ancestry-enriched biological pathways and inform ancestry-specific pharmacological targets. This trans-ancestral analysis strategy not only identified additional SLE-associated molecular pathways but, due to the underlying differences between AsA and EA in risk-allele frequencies, may enable a deeper understanding of the differences in the prevalence of SLE risk, severity, and clinical phenotypes. Such an understanding may motivate population-specific clinical trials and interventions.

### Results

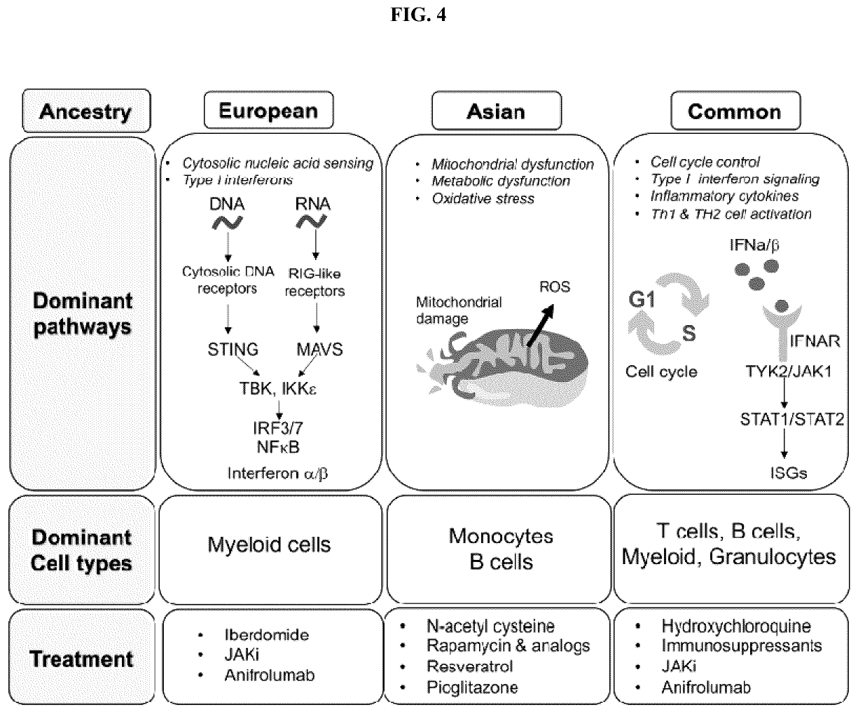

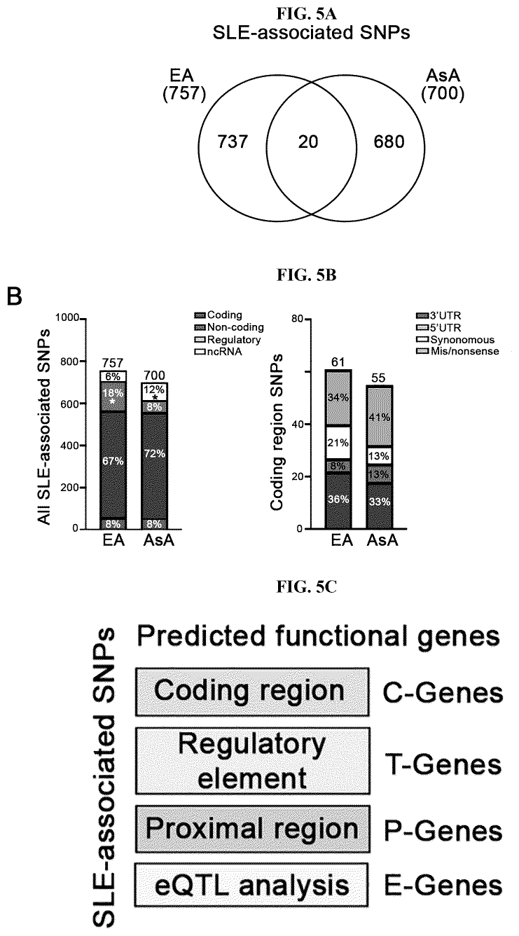

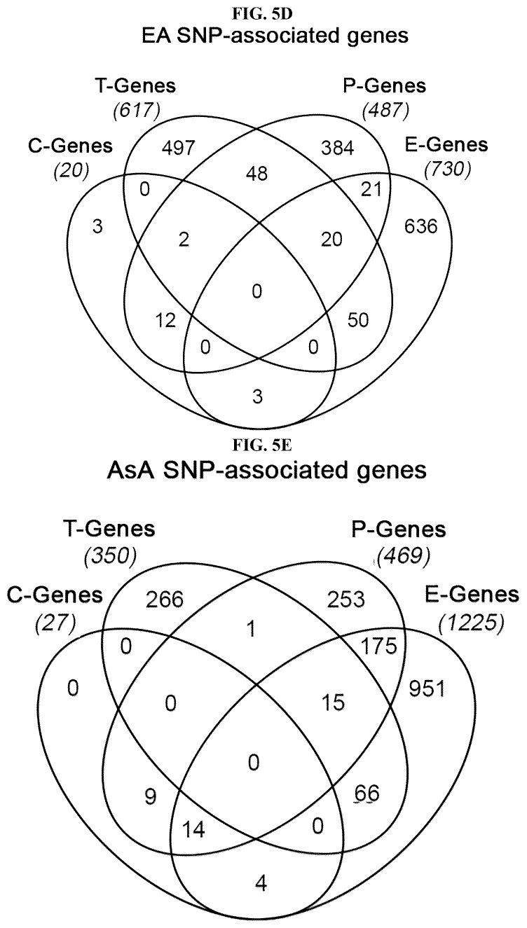

Identification of ancestry-dependent and independent non-HLA SLE-associated variants and downstream target genes. Despite the success achieved by GWAS in mapping polygenic disease risk loci in SLE, the biological implications of the majority of identified variants has remained unknown. To gain a broader view of how inherited genetic variation impacts disease risk, we took the global approach of integrating SNPs with a range of association significance to generate a cohort of predicted genes that could ultimately be pruned and mapped to functional pathways for analysis. Immunochip-based association analyses have identified 700 single-nucleotide polymorphisms (SNPs) reported as significantly associated with SLE in patients of East Asian (AsA) ancestry (6) and 757 SNPs associated with disease in European (EA) populations (FIG. 5A) (8). Twenty SNP associations (<1.5%) were shared between ancestries. In both ancestries, approximately 70% of SNPs were found in non-coding regions (intergenic and intronic), and 8% of SNPs were in coding regions (3′UTRs, 5′UTRs, synonymous and non-synonymous) (FIG. 5B and Table 13). AsA populations had a significantly higher percentage of SLE-associated SNPs in non-coding (nc)RNAs (lncRNA and miRNA), whereas EA populations had more SLE-associated SNPs located within regulatory regions, including enhancers, promoters, open chromatin, and transcription factor binding sites (FIG. 5B).

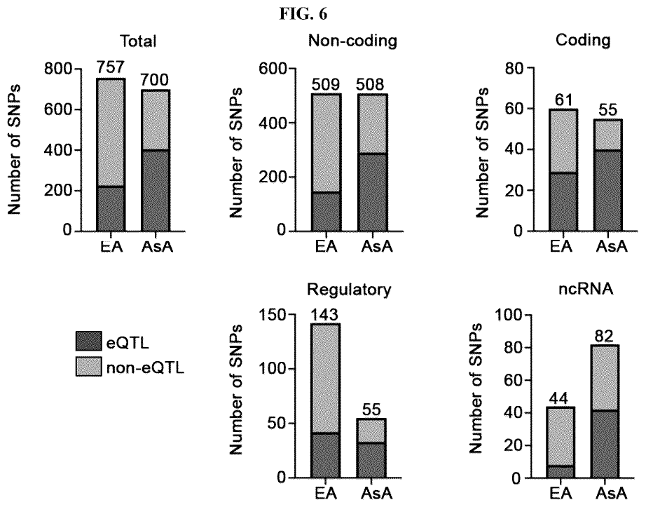

We used a bioinformatics-based approach to identify the most plausible genes affected by the SLE-SNP association. As previously described (11, 12), we first determined whether there was evidence that the SNP was an expression quantitative trait loci (eQTL) using the GTEx database (version 8) and the Blood eQTL browser (13). 226 EA- and 405 AsA-SLE-associated eQTLs linked to 730 and 1225 expression genes (E-Genes), respectively (FIG. 5C-E and Table 14). eQTLs were identified for nearly 60% of the total number of AsA Immunochip SNPs, compared to 29% of SLE-associated SNPs in the EA data (FIG. 6). Whereas the number of AsA eQTLs was higher across all genomic categories (coding, non-coding and regulatory regions as well as ncRNAs), the proportion of AsA SLE-associated SNPs linked to ncRNAs (51%; 42/82) was nearly three times higher than that observed in EA populations (18%; 8/44) (FIG. 6 and Table 13). Next, we sought to identify SNPs within distal and cis regulatory elements (e.g., enhancers and promoters), using the computational tools GeneHancer and HACER (Human ACtive Enhancers to interpret Regulatory variants), both of which connect regulatory SNPs with downstream target genes (T-Genes) (14,15). Together, GeneHancer and HACER identified 105 SLE-associated SNPs (59 EA, 36 AsA) overlapping distal regulatory elements or promoters predicted to impact the expression of 964 T-Genes (617 EA, 350 AsA) (FIG. 5C-E and Table 14). For variants located in coding regions, 44 SNPs (21 EA, 23 AsA) were associated with either non-synonymous or nonsense changes in 47 genes (C-Genes; 20 EA, 27 AsA) (Table 14). The remaining SNPs that were not linked to E-, T- or C-Genes were assigned to the closest proximal gene (P-Gene), identifying an additional 956 P-Genes (487 EA, 469 AsA) (Table 14).

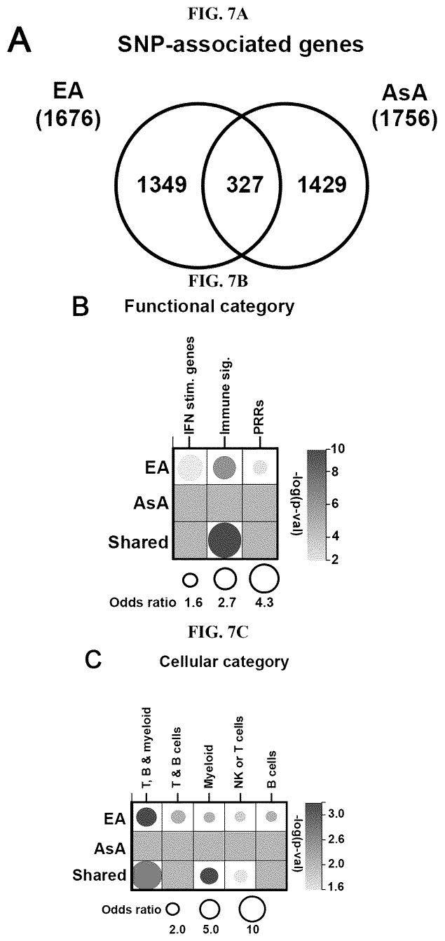

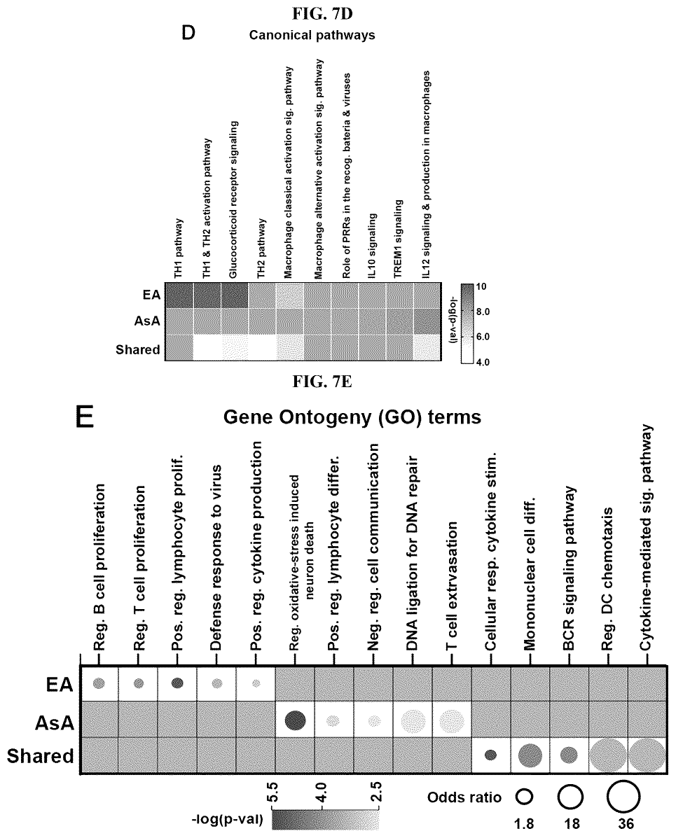

Overlapping EA and AsA SNP-linked E-, T-, C- and P-Genes are depicted in FIGS. 5D, and E, respectively. No genes were shared within all four groups within either ancestry, and we observed limited commonality between T-, P- and E-Genes, with only 20 genes shared among the three groups in EA and 15 genes shared in AsA. It is notable that of the total of 3,432 SNP-linked genes, <10% (327) overlapped between AsA and EA lupus cohorts (FIG. 7A).

Characterization of gene signatures: We next completed a series of bioinformatic analyses to determine the overall biological function of the 1349 genes associated with SLE in EA and 1429 genes associated with SLE in AsA, as well as the 327 SLE-associated genes common to both ancestries. Analysis of genes linked through EA revealed enrichment in processes related primarily to adaptive immune function, including the functional category for interferon stimulated genes, canonical pathways for TH1 and TH2 activation pathway and Macrophage classical activation signaling pathway, and GO terms for the Regulation of B cell proliferation (GO:0030888) and the Regulation of T cell proliferation (GO:0042129). Genes linked to SNPs associated with SLE in the AsA cohort were enriched in categories related to pathogen-influenced signaling, such as Role of PRRs in the recognition of bacteria and viruses, and the Positive regulation of lymphocyte differentiation (GO:0045621), as well as those representing more diverse biological functions, such as Regulation of oxidative stress-induced neuron death (GO:1903203) and DNA ligation involved in DNA repair (0051103). Shared genes were distributed in a range of adaptive and innate immune gene categories (FIGS. 7B, D, and E).

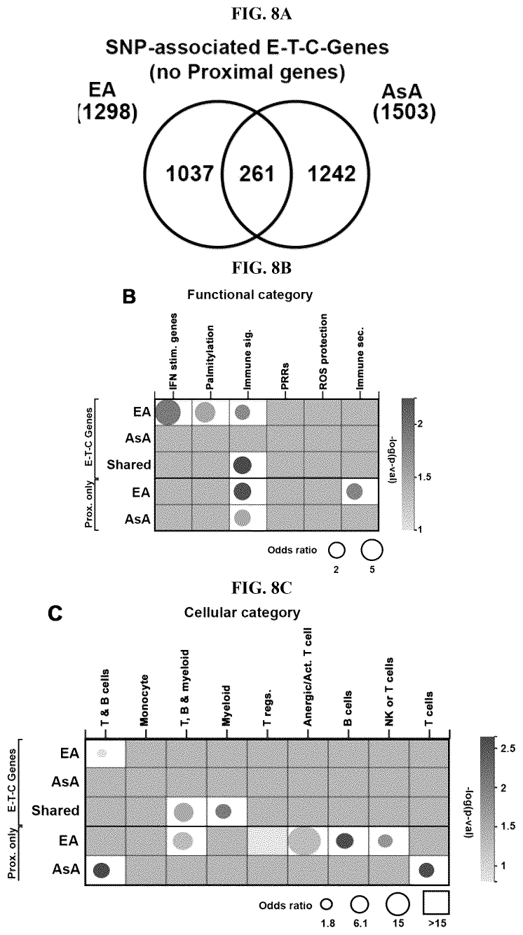

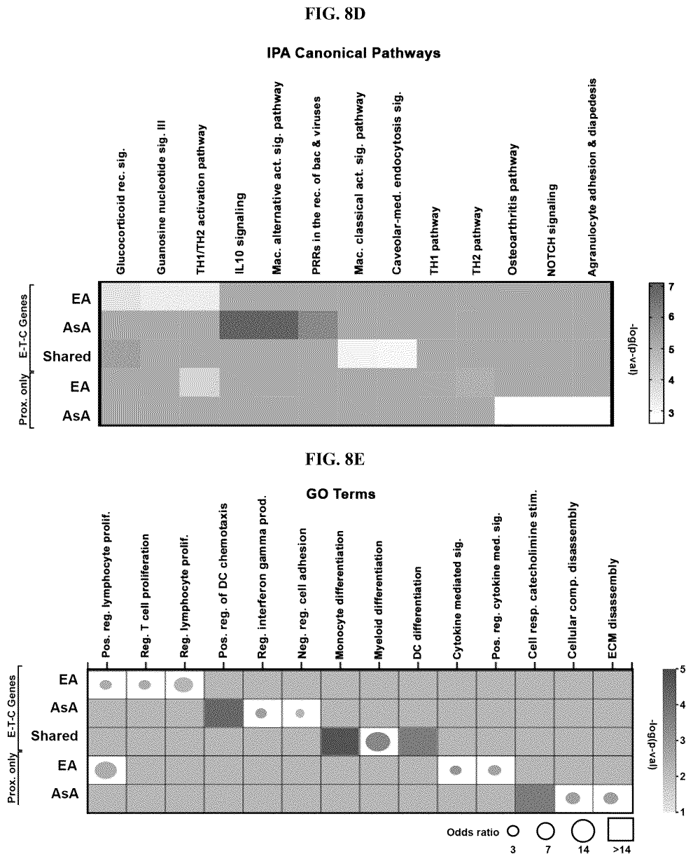

In addition, EA- and AsA-derived gene sets were examined using a clustering program that detects immune and inflammatory cell type signatures within large gene lists to identify dominant immune cell populations driving disease pathology within each ancestry (16). Consistent with our pathway analysis, EA exhibited strong enrichment in cellular categories for myeloid, T, and B cells, whereas SLE-associated genes in AsA were not enriched in any cellular category (FIG. 7C). Independent analysis of shared genes revealed enrichment in the T, B and myeloid, and the NK or T cell categories. Finally, parallel analyses examining P-Genes separately from E-, T-, and C-Genes were conducted to assess the potential overrepresentation of immune-based processes because of the Immunochip design bias (17). As expected, P-Genes (384 EA, 253 AsA) were enriched in immunologically-driven functional categories and pathways; exclusion of P-Genes resulted in only minor alterations to overall categorization in either ancestral background (FIGS. 8A-8E).

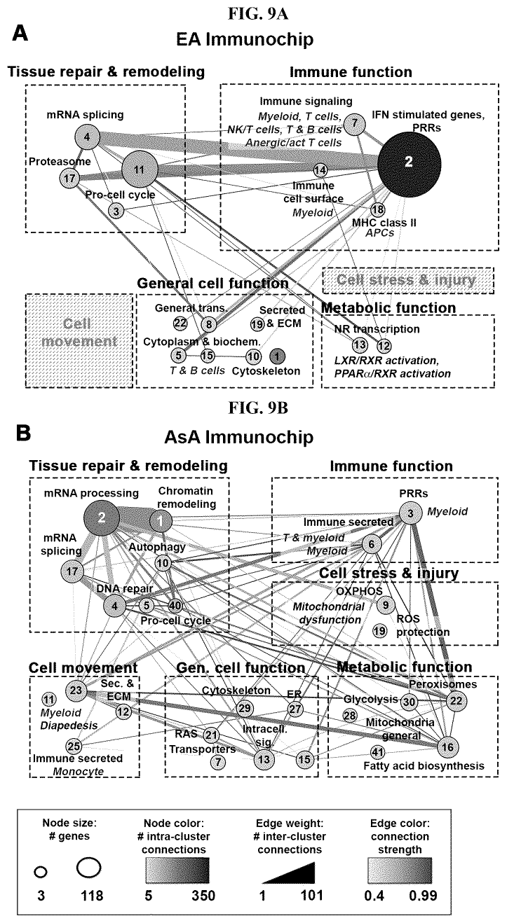

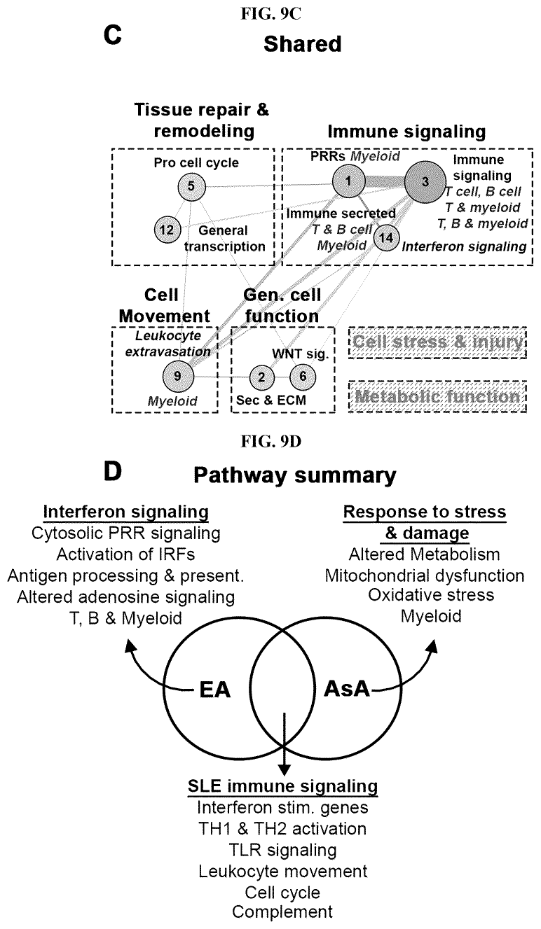

Delineation of signaling pathways identified by ancestry-specific SNP-associated genes. To assess ancestry-driven key signaling pathways in greater detail, ancestry-based protein-protein interaction (PPI) networks consisting of EA-associated, AsA-associated, or ancestry-independent genes were constructed using STRING, visualized in Cytoscape and clustered using MCODE (Table 15A-H). To provide an additional level of functional annotation, clusters contributing to overall immune function, tissue repair, mechanisms of cellular stress, cell motility, metabolic function or general cell function were grouped together. EA-associated genes were dominated by the functional category for interferon stimulated genes observed in cluster 2 (118 genes) (FIG. 9A), along with multiple canonical pathways related to the activation of pattern recognition receptors and downstream type I interferon signaling (Table 16). Cluster 7 revealed additional enrichment in lymphocyte activation and differentiation, such as the TH1 and TH2 activation pathway that was also represented in the shared gene network, and cellular enrichment for cells of myeloid and/or lymphoid origin. Notably, the EA-associated network lacked evidence of cell motility and cell stress/injury, whereas metabolic function was represented by clusters 12 and 13 enriched in retinoid X receptor activation (LXR/RXR activation, PPARα/RXRα activation) involved in the regulation of lipid metabolism, inflammation, and cholesterol bile acid catabolism. Pathways associated with SLE in AsA were indicative of a diverse range of biological processes with protein metabolic functions dominating clusters 2 and 17 (FIG. 9B), whereas clusters 3 and 6 were enriched in multiple canonical pathways related to cytokine production and signaling (Table 16). Interestingly, genes linked to SNPs associated in the AsA cohort did not include a unique interferon signature, but instead coalesced into multiple small clusters related to mitochondrial dysfunction (clusters 9 and 19) and metabolism, evident in clusters 16, 22 and 30. Additionally, AsA-associated gene clusters were enriched in chromatin remodeling found in cluster 1, along with evidence of cell motility (clusters 11, 12, 23 and 25). AsA cellular enrichment was dominated by monocytes and myeloid lineage cells.

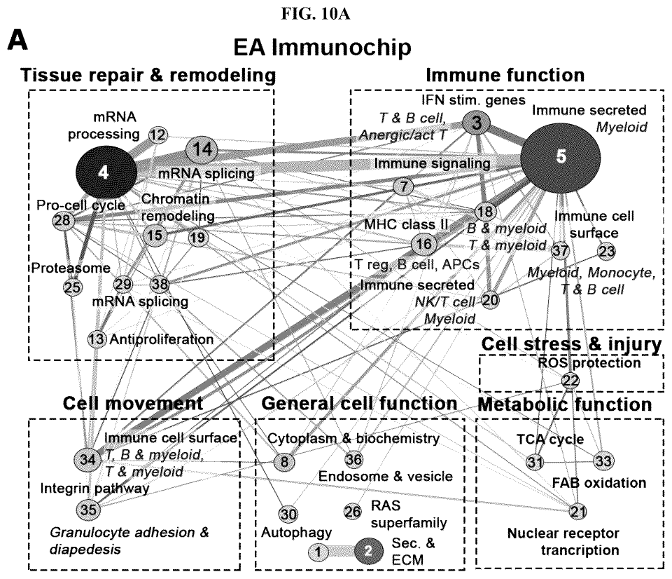

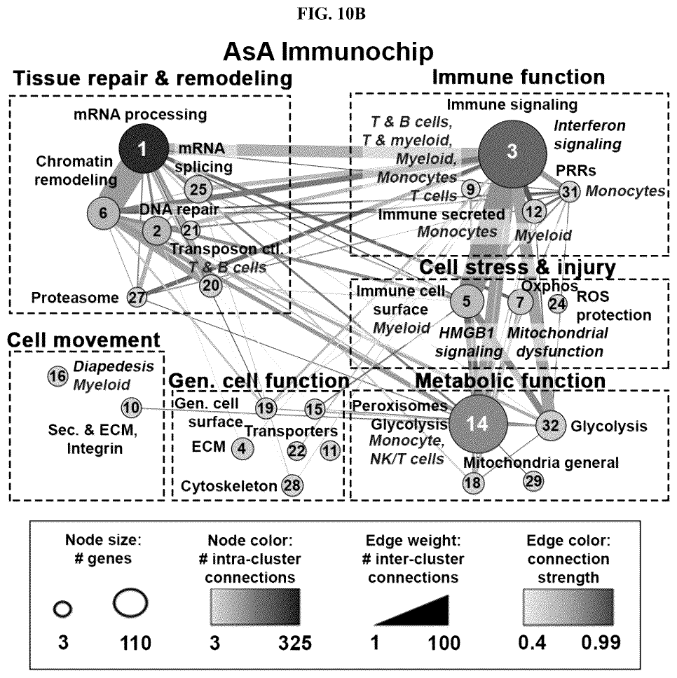

Pathways exemplified by SLE-linked genes in both EA and AsA appear to be a blend of the pathways enriched within each ancestry. Common pathways included Interferon signaling, TH1 and TH2 activation pathway, Complement system and Leukocyte extravasation (FIG. 9C and Table 17). FIG. 9D depicts a selection of both the unique and overlapping canonical pathways motivated by the EA-associated and AsA-associated gene sets. We also carried out a parallel series of bioinformatics analyses to determine the biological function of the full array of EA (1676) and AsA (1756) SNP-predicted genes, including those associated with both ancestries (FIG. 10 and Table 17). As expected, shared genes were evenly distributed throughout each large network and subsequent connectivity mapping revealed the addition of several new clusters to both the EA and AsA networks. For example, the full EA network gained several clusters contributing to cell motility enriched in integrin signaling and granulocyte diapedesis (clusters 34 and 35), whereas the enlarged AsA network gained multiple clusters enriched in immune function (clusters 9, 12 and 31) and interferon signaling (cluster 3), as well as enrichment in a more diverse array of cell types, including T and, B cells, neutrophils and NK/T cells. Both networks acquired a small cluster enriched in the functional category for reactive oxygen species (ROS) protection (EA cluster 22, AsA cluster 24) driven by the glutathione peroxidases GPX4, an antioxidant enzyme involved in ferroptosis (18), and GPX3 an ROS scavenger. Overall, the addition of the shared gene cohort to each network highlights many common pathways and biological functions, but still revealed ancestry-driven differences. Taken together, unique pathways identified via EA analyses appear dominated by immune-related signaling as well as T, B and myeloid cells, whereas those in AsA analyses are dominated by biological processes related to altered metabolism and mitochondrial dysfunction.

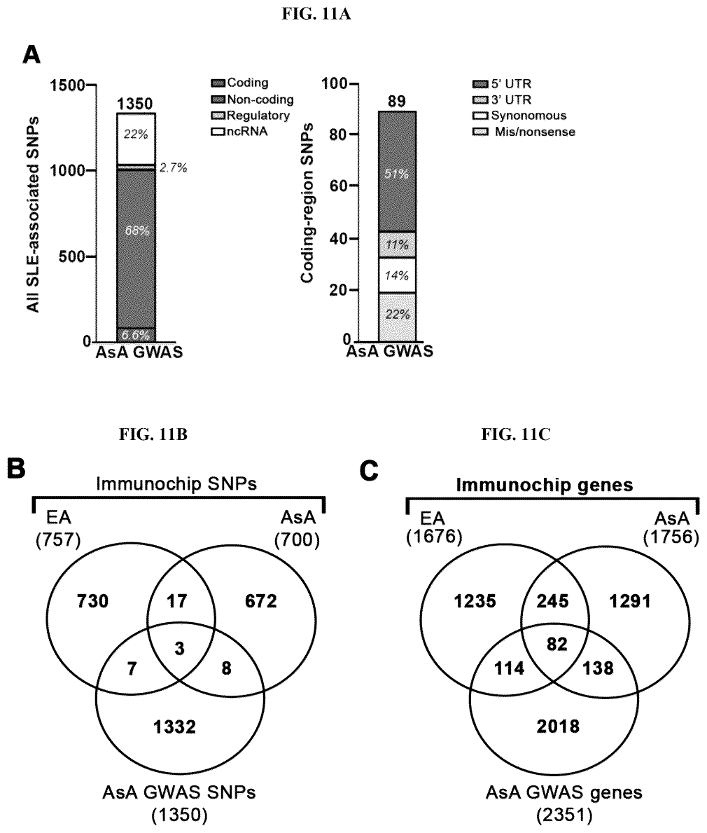

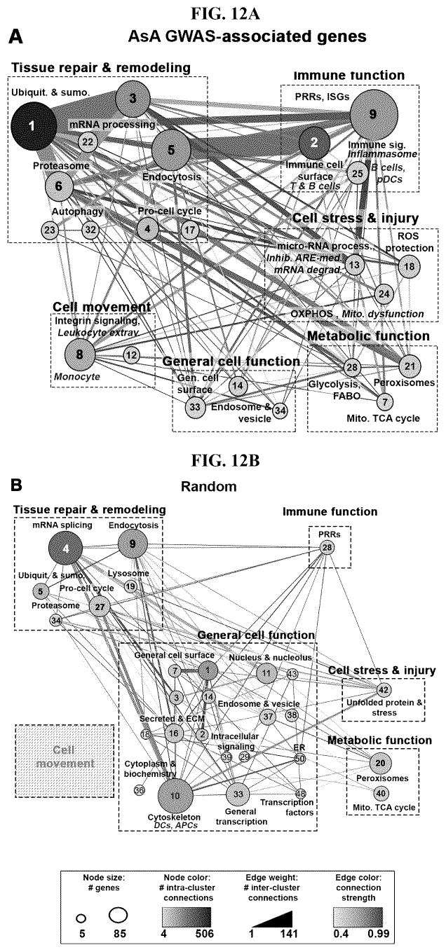

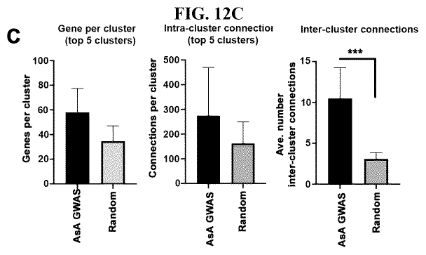

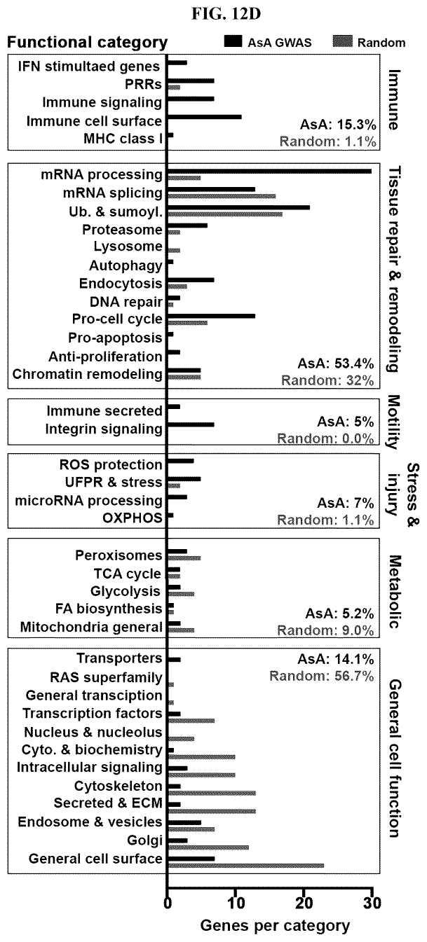

Validation of AsA-enriched molecular pathways using summary GWAS data. To test our pathway predictions, we combined summary data from previously reported GWA studies (7, 9) that identified 1350 SNPs associated with SLE in patients of East AsA ancestry (Table 18). Of these SNPs, 68% were located in non-coding regions, 6.5% were in coding regions, 2.7% were in regulatory regions and 22% were located within or proximal to non-coding RNAs (FIG. 11). Validation AsA-associated GWAS SNPs exhibited limited commonality when compared to Immunochip SNPs, with <1% of either EA- or AsA-associated Immunochip SNPs overlapping GWAS SNPs, and only 3 SNPs common to all 3 datasets (FIG. 11). We next applied our same bioinformatics-driven methodology to generate a validation gene cohort composed of 1321 E-Genes, 307 T-Genes, 17 C-Genes and 974 P-Genes (Table 19). Connectivity mapping of all validation genes were used to create PPI networks that were clustered as described above (FIG. 12A). Examination of each cluster revealed functional similarity to those derived from AsA Immunochip-associated genes. For example, clusters 1, 3, 4, 5 and 6 share hallmarks of tissue repair and remodeling exemplified by categories for mRNA processing, pro-cell cycle and protein degradation (proteasome, lysosome, endocytosis). Additionally, we observed smaller clusters (21, 27 and 28) representative of processes involved in metabolic function, and clusters (13, 18 and 24) characteristic of cell stress and injury, including the Inhibition of ARE-mediated degradation pathway and Mitochondrial dysfunction canonical pathways (Table 20). Cluster 9 contained a small interferon-stimulated gene signature consisting of IF127, IF144 and RSAD2 (Table 15A-H). Cellular categories were again dominated by monocytes, T cells, NK cells, B cells and plasmacytoid (p) DCs and are consistent with findings observed with AsA Immunochip-associated genes. In contrast to AsA-associated genes, where we observed large, highly connected clusters, an equivalent cohort of apparently random genes generally formed smaller clusters, exhibited fewer intra- and inter-cluster connections, and were primarily enriched in functional categories mostly related to basic cell function (general cell surface, secreted and ECM) (FIGS. 12B and C). As shown in FIG. 12D, which displays the number of genes (and percentage of total genes) assigned to each functional category, random genes are skewed toward general cell function, whereas AsA-associated genes are more prevalent in the overall immune (15.3% of genes), tissue repair (53.4%) and cell stress (7%) categories. The random gene network also lacked evidence of cell movement and the diversity of cellular enrichment identified from AsA SNP-associated genes (FIG. 12B).

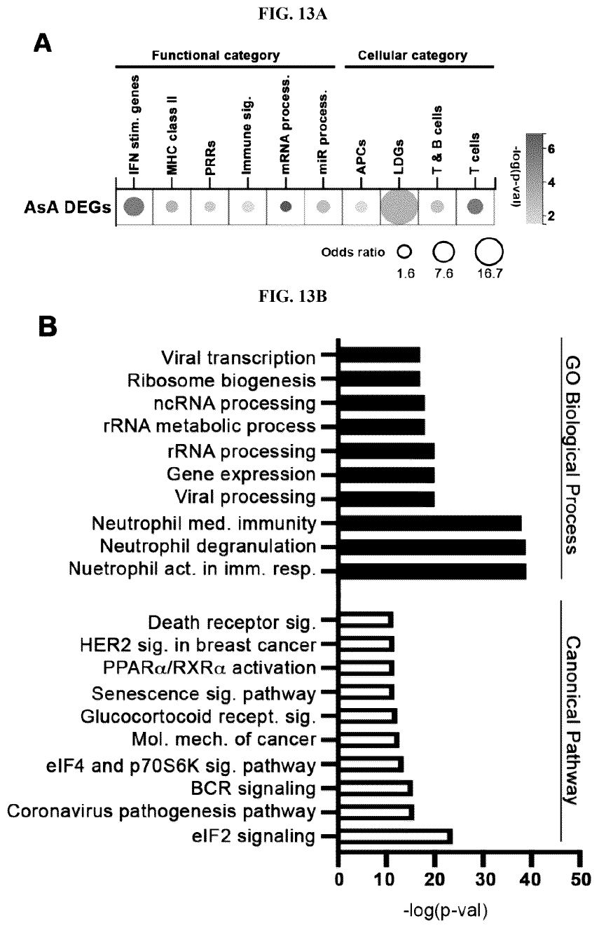

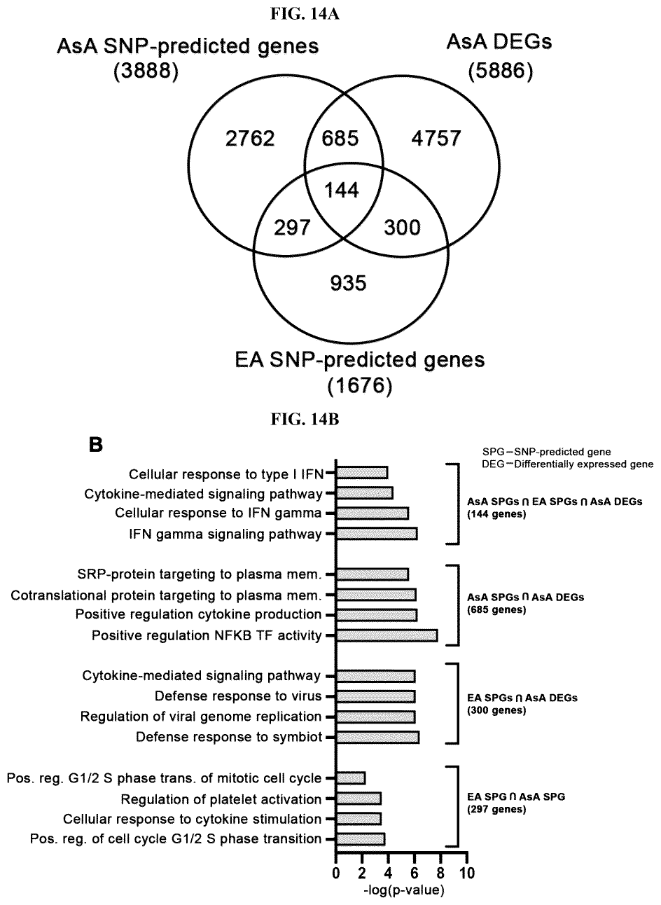

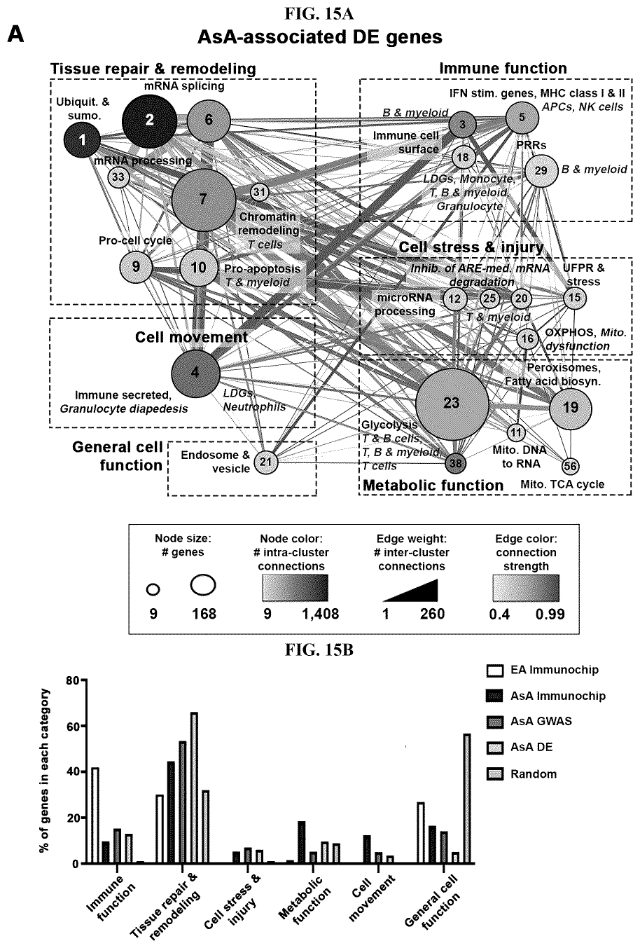

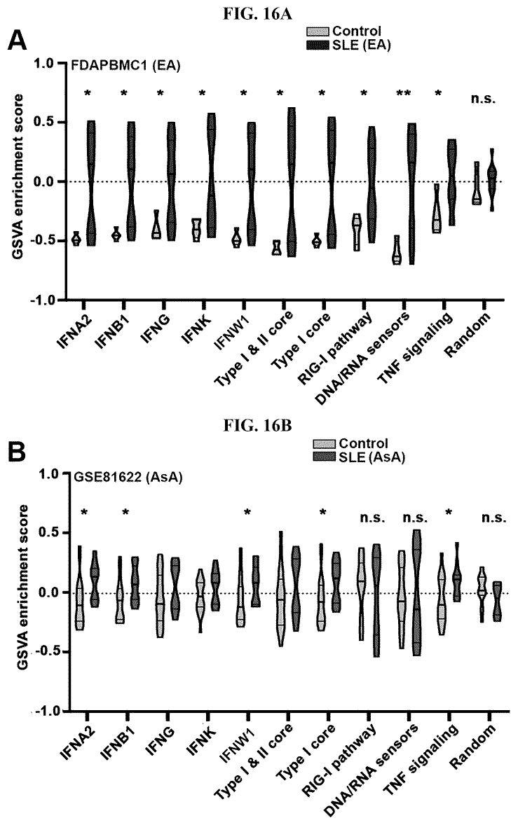

Validation of AsA-enriched molecular pathways using gene expression data. SNP-based pathway predictions were also tested in differential gene expression analyses from whole blood samples collected from AsA SLE patients with active disease and renal involvement compared with healthy controls (E-MTAB-11191, Table 23). Overall, differential expression (DE) analysis revealed 5886 DE genes (DEGs) enriched in functional categories for interferon stimulated genes, gene expression, RNA processing and metabolism (FIGS. 12A-D and FIGS. 13A-B). A total of 685 AsA and 300 EA SNP-predicted genes were shared with AsA SLE DEGs, and 144 genes, representative of type I and type II interferon signaling, were shared among all three groups (FIG. 14). Genes common to AsA DEGs and AsA SNP-predicted genes were enriched in RNA processing and translation, whereas DEGs shared with EA SNP-predicted genes were specifically enriched in type I interferon/cytokine signaling. Connectivity mapping and pathway analysis of AsA DEGs again revealed striking commonality with AsA SNP-associated genes from both the Immunochip and summary GWAS, exemplified by cluster enrichment in RNA splicing/processing, ubiquitylation, chromatin remodeling and metabolic function (glycolysis, TCA cycle, fatty acid biosynthesis and peroxisomes), and canonical pathways for Spliceosome cycle (cluster 2), Inhibition of ARE-mediated RNA degradation (clusters 12, 20 and 25), and Mitochondrial dysfunction (cluster 16) (FIG. 15A and Table 21). The distribution of DE genes in AsA for each overall category were also similar to that observed with AsA-associated genes (Immunochip and GWAS) but differed from both the EA-Immunochip predicted or random genes (FIG. 15B).

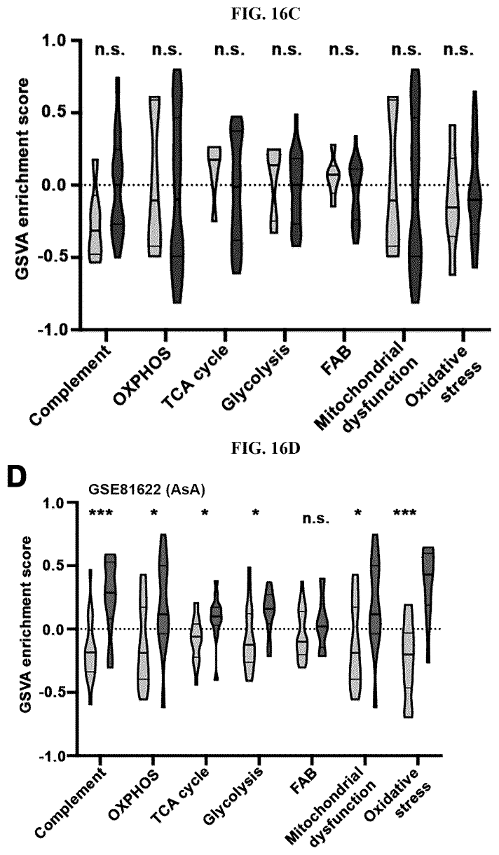

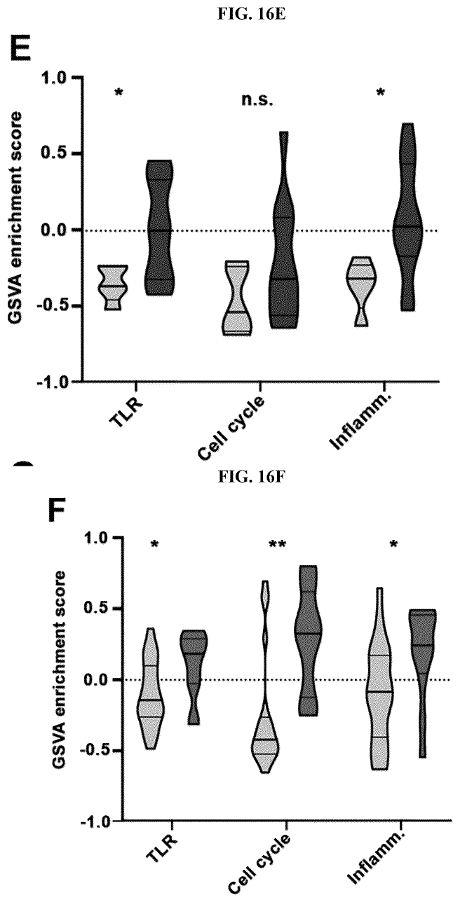

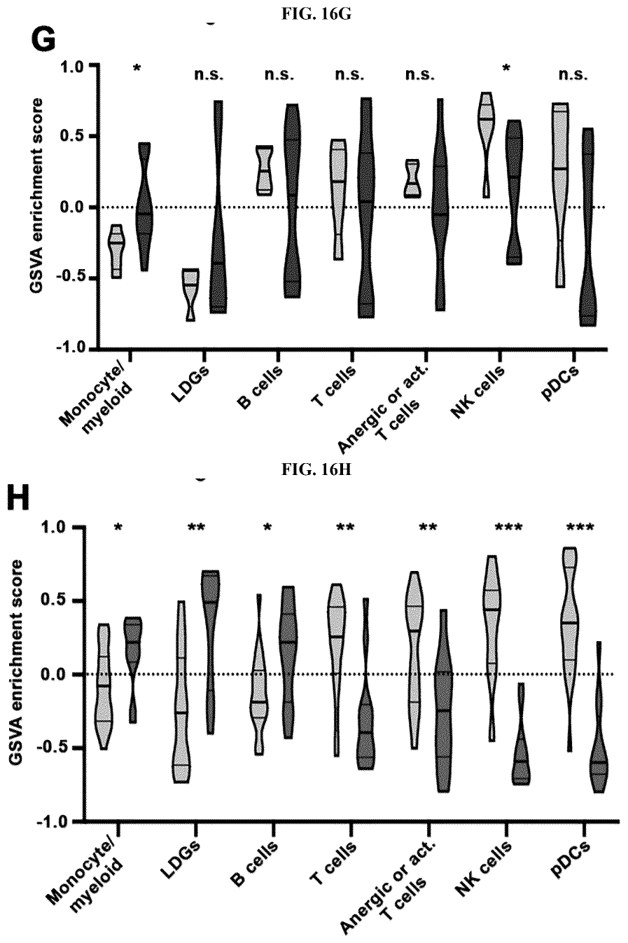

To test the pathway predictions, Gene Set Variation Analysis (GSVA) (19) was applied to determine the relative enrichment of gene signatures identified in peripheral blood mononuclear cell (PBMC) samples from SLE patients (EA and AsA) and controls (Table 22). In FDAPBMC1, a dataset composed of EA patients (Table 23), all 7 IFN gene signatures (IGS) and signatures for the RIG-I pathway and DNA/RNA sensors were strongly enriched in SLE PBMCs compared to controls (FIG. 16A). In contrast, only the signatures for IFNA2, IFNB1, IFNW1 and the Type I core were enriched in SLE PBMCs from AsA patients in GSE81622 (FIG. 16B). GSVA using a random group of genes did not separate SLE from controls in either dataset.

GSVA enrichment scores using signatures for complement activation and metabolic pathways, including mitochondrial oxidative phosphorylation (OXPHOS), the TCA cycle and glycolysis were able to separate AsA SLE patients, but not EA patients, from healthy controls (FIGS. 16C, and D). Consistent with our predictions, AsA SLE patients exhibited significant enrichment in signatures for mitochondrial dysfunction and oxidative stress. As shown in FIG. 16E-H, enrichment of a number of pathways and cell types in both EA and AsA SLE cohorts were noted, including TLR signaling, inflammasome, TNF signaling and monocyte/myeloid lineage cells, whereas AsA patients exhibited additional enrichment in pro-cell cycle, low density granulocytes (LDGs) and B cells. Varying degrees of T cell/NK cell lymphopenia were evident in both ancestral populations.

### Differential Inflammatory Cell Metabolism and Activation Status by Ancestry.

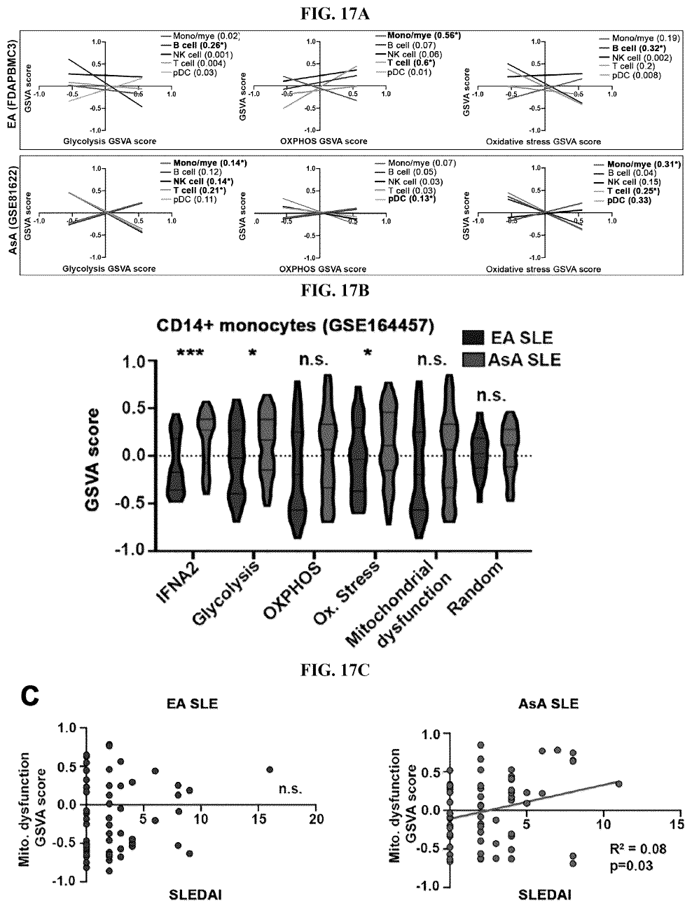

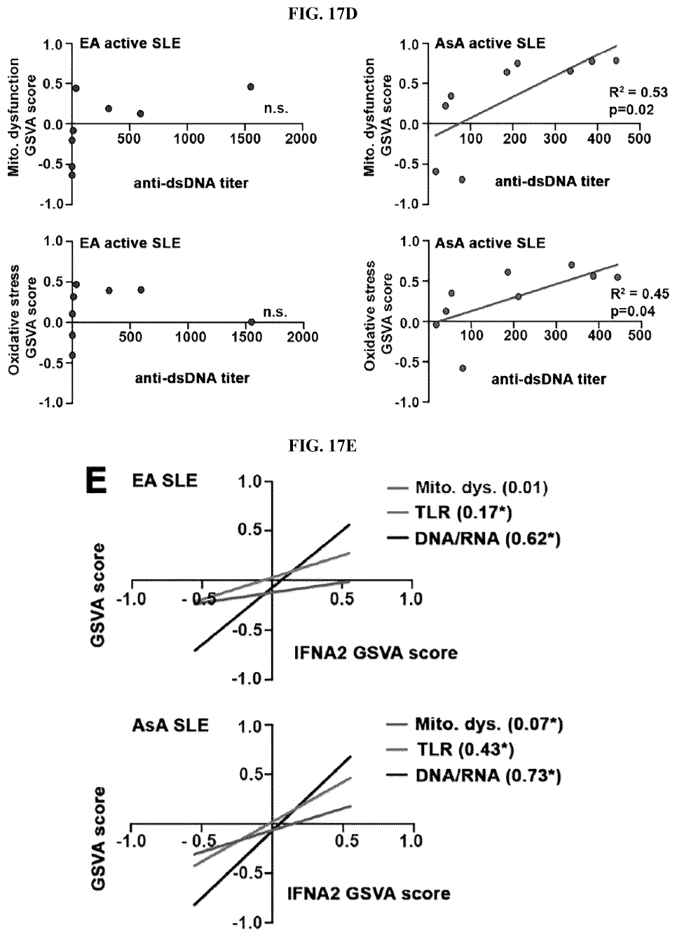

Because dysfunctional metabolic reprogramming can directly influence and exacerbate defective immune responses, we carried out linear regression analysis between the GSVA scores for individual cell signatures and the glycolysis and oxidative phosphorylation gene signatures to interrogate the metabolic status of immune cells from EA and AsA PBMC datasets. Enrichment scores for the glycolysis gene signature in AsA SLE patients exhibited positive correlation with both the monocyte/myeloid (R2=0.14, p=0.03) and B cell signatures (R2=0.12, p=0.051) (FIG. 17A). In contrast, glycolysis was not associated with gene signatures representing monocyte/myeloid cells, T cells or NK cells from EA patients, whereas the B cell category exhibited significant negative correlation (R2=0.26, p=0.03). The gene signature for oxidative phosphorylation lacked positive correlation with all cell types in AsA patients, but did exhibit significant correlation (R2=0.6, p=0.0003) with T cells in individuals of EA ancestry (FIG. 17A).

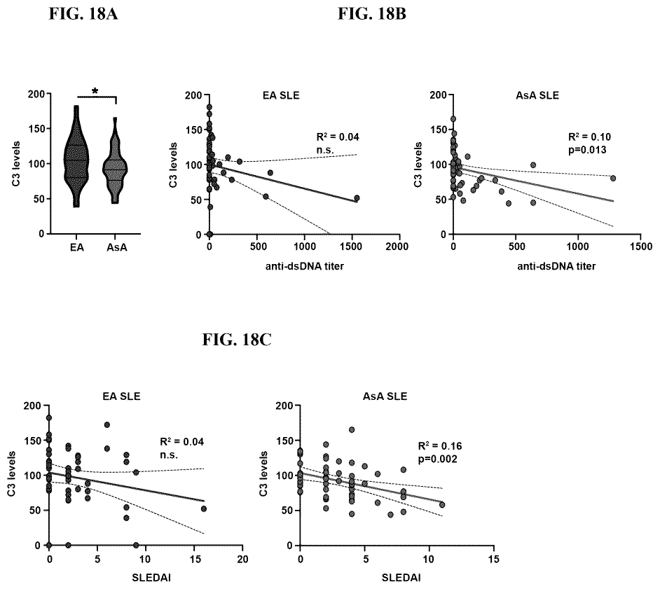

Since altered immune cell metabolism is associated with heightened oxidative cell stress responses in SLE20, we used the same linear regression approach to test the relationship between different immune cell types and GSVA enrichment scores for oxidative stress (FIG. 17A). Monocyte/myeloid cells from AsA patients were the only cell type demonstrating a significant positive relationship with the gene signature for oxidative stress (R2=0.3, p=0.0008). These results were confirmed in purified CD14+ monocytes isolated from EA and AsA SLE patients (California Lupus Epidemiology Study, CLUES; GSE16445721) (Table 23) showing significant enrichment of gene signatures for oxidative stress, glycolysis and IFNA2, along with a trend toward elevated OXPHOS and mitochondrial dysfunction, specifically in AsA SLE patients (FIG. 17B). Given that enhanced oxidative stress and concomitant mitochondrial dysfunction have been shown to correlate with disease activity, DNA damage, and autoantibody production (22,23,24), we next examined the potential link between enrichment scores for mitochondrial dysfunction and SLE disease activity index (SLEDAI) score in these patients. GSVA scores for mitochondrial dysfunction demonstrated a significant positive relationship with SLEDAI in AsA but not EA SLE monocytes (FIG. 17C). Similarly, enrichment scores for oxidative stress and mitochondrial dysfunction showed a significant positive correlation with anti-dsDNA titers among AsA SLE patients with active disease (SLEDAI≥6) compared to EA patients (FIG. 7D). In addition, overall complement C3 levels were lower in AsA patients and demonstrated a significant negative correlation with both anti-dsDNA and SLEDAI (FIG. 18) in accordance with the observation that complement depletion and anti-dsDNA antibodies are often associated with elevated disease activity (25).

Finally, given that mitochondrial dysfunction and nucleic acid sensing are potent inducers of interferons (26), we tested for a relationship between IFNA2 enrichment scores and enrichment scores for the mitochondrial dysfunction, DNA/RNA sensors and TLR pathways (FIG. 18E). In EA patients, GSVA scores for the DNA/RNA sensors (R2=0.62, p<0.0001) and TLR pathway (R2=0.17, p=0.0013) signatures, but not mitochondrial dysfunction, were associated with IFNA2. In contrast, all three signatures exhibited a positive, significant correlation with IFNA2 in AsA patients.

### Discussion

SLE is a multisystem autoimmune disorder with a strong genetic contribution. The incidence of SLE varies widely across populations, with individuals of Asian, Hispanic and African ancestry demonstrating a three- to four-fold increase in disease prevalence compared to their European counterparts (27). The advent of candidate gene, Immunochip and genome wide association studies (GWAS) has transformed our understanding of SLE genetics. However, it remains unclear how genetic ancestry contributes to the incidence, clinical heterogeneity and variation in disease outcomes among SLE patients. Specifically, AsA patients develop SLE at a younger age and with more severe manifestations, including lupus nephritis (LN) (28). Whereas increased genetic risk burden in AsA individuals has been hypothesized to account for the increased prevalence of SLE in this population (9), it does not provide adequate explanation for accelerated disease progression, variation in treatment response, or more extensive organ damage, especially with regard to the development of LN. Our observations suggest that, in addition to higher risk load, underlying differences at the genetic level may significantly influence the dominant biological pathways operative within each ancestry. Here, we show that by identifying a comprehensive list of genes implicated by GWAS and linking them to biologic pathways, we can provide a broader perspective on the genetic influences of SLE. It may also inform on health disparities particular to specific ancestries.

To accomplish this, we adopted a similar strategy to that recently described (11), employing statistical and computational analyses along with data acquired from functional genomic assays to map the overall gene expression landscape of SLE and further define the disease-associated pathways. It is important to highlight that the SNPs examined here are generally present in both EA and AsA populations, but differences in allele frequencies suggest some SNP-predicted genes and pathways may be more influential in one ancestry over another. Rather than focusing on only a very limited number of top associations, this approach used a less stringent significance threshold to allow for the integration of more SNPs that could ultimately be pruned and mapped to a larger cohort of genes and pathways. Using SNPs discovered via Immunochip-based association studies, eQTL analysis identified 631 SNPs associated with 1955 E-Genes (730 EA, 1225 AsA). In the Asian ancestry studies, nearly 60% of SNPs were eQTLs, compared to 29% in EA. While eQTLs represented a higher proportion of SNPs associated with SLE in AsA across all genomic functional categories, it was of note that eQTLs linked to ncRNAs were nearly 3 times as frequent in AsA compared to EA patients. The disparity in distribution likely represents the heterogeneous genetic disposition uniquely affecting EA and AsA SLE patients. ncRNAs are a class of mRNA-like transcripts, typically >200 nucleotides in length, that lack protein coding potential and serve as important regulators of gene expression by actions at the transcriptional, post-transcriptional and post-translational levels (29). Several ncRNA eQTLs identified here were associated with anti-sense RNA E-Genes, including IFNG-AS1 and IL12A-AS1, both of which are involved in the regulation of their cognate sense protein-coding genes (30, 31). Increasing evidence points to an important role for ncRNAs in the differentiation, polarization and activation of both myeloid and lymphoid lineage immune cell types (32). Furthermore, abnormal ncRNA expression is associated with mitochondrial dysfunction-induced oxidative stress in a number of pathological conditions, including SLE (33, 34, 35).

Overall, genes linked to SNPs associated with SLE in AsA cohorts were enriched in processes related to leukocyte migration, PRR signaling and RNA processing, and further detail provided by protein-protein interaction network and pathway analysis revealed multiple clusters enriched in translation/RNA processing, metabolic function, chromatin remodeling, cell stress and mitochondrial dysfunction. In contrast, these pathways, particularly mitochondrial dysfunction and cellular stress responses, were absent from the network analysis of EA SNP-associated genes. Instead, SLE-associated genes in EA data tended to be heavily influenced by immune processes, including the Role of RIG-I in antiviral innate immunity, Antigen presentation, and the SLE in T cell signaling pathway, as well as the functional category for interferon stimulated genes. Cellular enrichment categories were overwhelmingly dominated by T cells, B cells and myeloid cells, and is consistent with previous findings showing increased myeloid/monocyte gene signatures in EA ancestry independent of medication usage (i.e. SLE standard of care drugs) and autoantibody production (36). SNP-predicted pathways common to both EA and AsA were also enriched for myeloid and lymphoid lineage signaling (i.e. TH1/TH2 activation signaling and IL12 signaling and production in macrophages), along with the well described role of interferons in SLE.

To test our Immunochip SNP-predicted pathway-based findings, we turned to summary GWAS data from AsA SLE patients to create a second “validation” cohort of genes. While little numerical overlap between Immunochip SNPs and those derived from the AsA validation meta-analysis GWAS was observed, network-based analysis of SNP-predicted genes demonstrated a striking resemblance between pathways predicted by the Immunochip and those from the validation gene set, with multiple large clusters enriched in mRNA processing, degradation and nucleic acid sensing, along with smaller clusters enriched in metabolic activity and mitochondrial dysfunction. Conversely, network generation from an equivalently-sized random gene cohort contained smaller clusters, fewer intra- and inter cluster connections and exhibited little functional similarity with pathways predicted by AsA- or EA-associated genes. Nonetheless, consistent with our AsA Immunochip findings, SNPs within or proximal to ncRNAs were also highly prevalent in the validation dataset, accounting for 22% (299/1350) of total SNPs.

Recently, Wang and colleagues used ancestry-dependent and trans-ancestry meta-analyses to identify 38 novel non-HLA SLE loci (37). Six disease variants were associated with SLE exclusively in East Asian populations of which 5 (HIP1, TNFRSF13B, PRKCB, DSE and PLD4) were linked to genes also identified here. Two of these genes are involved in antibody production, including TNFRSF13B that encodes the receptor for BAFF and plays a critical role in B cell development and survival, and PRKCB, a protein kinase C family member that regulates B cell activation via BCR-induced NF-κB activation (38). These data suggest that different mechanisms may exist for antibody regulation between AsA and EA populations, whereby pathogenic antibody levels observed more frequently in non-Europeans may contribute to the higher prevalence of SLE in these populations. In fact, data presented here showing enrichment of B cell signatures and positive correlation between B cells and glycolysis may be indicative of increased B cell activation specifically in SLE patients of Asian ancestry. Nonetheless, by integrating all SLE SNP association-predicted genes into functional pathways, we have been able to detect additional differences contributed by ancestry such as those related to stress responses and metabolic dysfunction.

Pathway observations based on genetic findings were also tested in differential gene expression data. The biological pathways determined by AsA-associated DEGs from whole blood samples exhibited impressive similarity to those coupled to AsA SNP-associated genes and included evidence of metabolic and mitochondrial dysfunction, oxidative stress, cell death and gene expression/chromatin remodeling related processes. Furthermore, these results were validated via GSVA analysis in a second expression dataset from PBMCs showing that gene signatures for these processes were specifically enriched in samples from AsA, but not EA SLE patients. Despite the differences in EA and AsA revealed by network pathway analysis, SNP-predicted cellular enrichment common to both ancestral backgrounds included a wide variety of immune cell types, including APCs, T cells, B cell, myeloid, monocytes, LDGs and neutrophils. Additionally, while B cells and LDGs were enriched specifically in AsA SLE patients, the GSVA-based expression pattern demonstrating enrichment in monocyte/myeloid cells and reduced expression of T/NK and APCs was similar between EA and AsA SLE patients and is consistent with the involvement of these cell types in lupus pathogenesis.

Studies examining whether ancestry affects the clinical phenotype of SLE are complicated by the overwhelming heterogeneity of disease manifestations, especially with respect to organ involvement. Nonetheless, many genetic polymorphisms have been associated with specific phenotypes, autoantibody profiles, and/or clinical outcomes in SLE. For example, FcG receptor subtypes, such as FCGR3B have been significantly associated with LN and the presence of pathogenic autoantibodies, although it remains unclear whether there is a genetic basis for end-organ involvement based on ancestry (39). FCGR3B is almost exclusively expressed by neutrophils and low copy number is associated with glomerulonephritis (39). The SNP-predicted pathways described here suggest the presence of different biological mechanisms driving SLE. Importantly, we observed differential enrichment of these pathways in EA and AsA SLE data and thus these pathways may help explain some of the heterogeneity in SLE prevalence and severity across ancestral populations. This is not without precedent, as SNP-predicted genes from African Ancestry (AA) SLE patients identified pathways related to B cell signaling11 and these findings are consistent with reports showing that AA patients experience increased B cell activation and plasma cells compared to their EA counterparts (36, 40, 41). Dominant pathways identified by AsA-associated SNPs and verified by gene expression data include those linked to mitochondrial and/or metabolic dysfunction and subsequent oxidative injury. Mitochondrial components can directly stimulate immune receptors by acting as damage-associated molecular patterns (DAMPs) (42). They also function as an important source for reactive oxygen species (ROS), as well as facilitate inflammasome activation (42). In SLE patients, defective mitochondrial function can increase oxidative stresses characterized by increased lipid peroxidation, elevated ROS production and decreased levels of antioxidant enzymes, such as superoxide dismutase (SOD), catalase (CAT) and glutathione peroxidase (GPx) (22). In fact, enhanced mitochondrial ROS synthesis in SLE has been shown to directly contribute to type I IFN responses (43), findings that are consistent with our results showing a significant correlation between the gene signatures for mitochondrial dysfunction and IFNA2 driven by the AsA analyses.

Importantly, mitochondrial dysfunction and associated oxidative damage has also been shown to be a leading factor in the development of chronic and acute renal diseases (44). Lupus nephritis (LN) is a major risk factor for overall morbidity and mortality in SLE. Previous work has shown that SLE patients of Asian descent are at significantly higher risk for the development of LN (45), whereas European genetic ancestry was found to be protective against renal disease (46). In accordance with these findings, enrichment scores for mitochondrial dysfunction and oxidative stress significantly correlated with anti-dsDNA titers in AsA SLE patients with active disease compared to EA patients. These observations are intriguing in light of recent findings by Tsai and colleagues indicating cross-talk between oxidative stress and ncRNAs can trigger autoimmune reactions and perpetuate tissue damage in SLE patients (33). Although the reason for the disparity in renal involvement between ancestral backgrounds has not been fully delineated, we could posit that differential expression of ncRNAs in kidney tissues may contribute to the significant immune dysregulation affecting Asian SLE patients. Whether this might be related to expression of specific regulatory ncRNAs or is a consequence of overall ncRNA burden will require further examination. However, in support of the former, several groups have reported aberrant cell type specific activation linked to the altered expression of non-coding microRNAs, including miR-31, miR145 and miR224 involved in T cell activation, that may be participating in LN pathophysiology (47). Although these and other studies examining ncRNAs were conducted using samples from patients primarily of Asian ancestry, their results do not obviate the possibility that similar effects occur in EA SLE patients. This highlights a major limitation of the current study: there is no direct evidence that Asian patients experience a higher degree of mitochondrial dysfunction and oxidative stress-induced tissue damage compared to other ancestral backgrounds. As such, the relationship between altered ncRNA expression, immune dysfunction and tissue injury remains unclear. Nonetheless, genetic and gene expression evidence provided here suggests that AsA ancestral populations may be predisposed to altered cell stress responses where excessive mitochondrial oxidative stress derived ROS and RNS may trigger autoimmune reactivity, increasing cell death and promoting destructive inflammatory reactions in susceptible individuals.

Our genetic and gene expression analyses suggest that metabolic dysfunction is a key feature more prevalent in individuals of Asian compared to European ancestry. Reprogramming of immune cell metabolism is required to sustain the energy demands of effector functions, such as differentiation, clonal expansion, secretion of proinflammatory mediators, phagocytosis, and chemotaxis (48). Metabolic dysfunction is common in kidney disease and recent work by our group has demonstrated that altered metabolic function in lupus-affected tissues (kidneys and skin) reflect damage induced by myeloid cell infiltration (16). In myeloid lineage cells (monocyte/macrophages), enhanced glucose metabolism, either via glycolysis (characteristic of M1 macrophages) or OXPHOS (characteristic of M2 macrophages) is essential for cell survival, proliferation and to sustain various effector responses (49). Regression analysis using PBMC and purified CD14+ monocytes isolated from SLE patients revealed a significant positive correlation between monocyte signatures from AsA subjects and glycolysis, but not OXPHOS, suggesting they are likely to be metabolically M1 in nature. Glycolysis was also correlated with B cells in AsA individuals suggesting that B cells, along with monocyte/myeloid cells in this patient population, maintain an activated phenotype.

The computational and experimental approaches are inferential. By attempting to provide a comprehensive translation of all GWAS findings, it remains challenging to determine those genes that are causal and those that may be considered false-positive associations. To address this, PPI networks and unsupervised MCODE clustering based on interaction strength help exclude those genes lacking strong connections within or between similarly functioning clusters to uncover biologically-relevant pathways. In comparison to networks generated from random SNPs and genes, the densely connected PPIs and highly significant gene-set annotation enrichments displayed in the current manuscript are clearly distinct from background noise and often implicate unexpected molecular pathways that may be ancestrally-motivated.

Initial genetic findings were based on the Immunochip which was specifically constructed for deep replication of major autoimmune and inflammatory diseases and fine-mapping of established GWAS loci (17). As this platform was designed for use in European populations, it may have introduced considerable bias, especially with respect to the dominance of immune and interferon-mediated signaling pathways observed in our EA analyses. Given that type I IFN is a primary pathogenic factor in SLE regardless of ancestral background, additional analyses, including an examination of pathways predicted from summary AsA GWAS, differential gene expression from AsA whole-blood samples and GSVA enrichment of interferon gene signatures in AsA patients confirmed the well-established association between SLE and IFN across ancestral populations. Nonetheless, the specific triggers of IFN production and the mechanisms by which interferon signaling perpetuate the cycle of autoreactive cells and autoreactive antibody production are not completely clear, and evidence presented here suggest that different mechanisms exist for autoantibody and IFN regulation between EA and AsA populations. Finally, the ability to map SNPs to implicated genes is limited to known SNP-to-gene relationships included in Ensembl's variant effect predictor (VEP), Genotype-Tissue Expression (GTEx) and Human ACtive Enhacer to interpret Regulatory variants (HACER) databases. However, of all SNP-predicted genes, only those encoding proteins included on the STRINGdb platform were incorporated into PPI interaction networks. While this is useful for filtering out unrelated genes, it excludes the large number of non-coding genes implicated in our SNP-to-gene predictions, an important consideration given the growing evidence highlighting the contributions of long non-coding RNAs and microRNAs in SLE34.47. Similarly, the ability to annotate gene clusters functionally is limited and potentially biased by the data underlying the numerous enrichment platforms used in our pathway analyses. It is for this reason that multiple platforms were used, including Ingenuity Pathway Analysis (IPA), EnrichR, which pools a myriad of public databases, and cell (IScope) and functional (BIG-C) analytic tools, to obtain orthogonal and reproducible annotations.

In conclusion, the SNP-associated predicted genes and gene expression profiles outlined here implicate fundamental differences in lupus molecular pathways enriched in EA and AsA ancestral populations. Our findings suggest that while certain pathways may be enriched in one ancestral population over another, it is important to note that those pathways may not be active within every patient of a given ancestry and may be active in a patient from different ancestry. Systems bioinformatics and assessment using gene signature enrichment analyses revealed alterations in cellular metabolism and cell stress signatures that may be more prevalent in patients of Asian ancestry. Whereas treatment strategies aimed at restoration of metabolic and/or antioxidant pathways are not straightforward, the current findings suggest that targeting metabolic dysfunction may hold promise for AsA patients who respond poorly to conventional therapies. Indeed, mTOR pathway modulators such as N-acetyl cysteine and rapamycin appear to be viable therapies for reducing disease activity (50,51). Recently, pioglitazone, a peroxisome proliferator-activated receptor (PPARg) agonist, was found to ameliorate nephritis symptoms in lupus-prone animals (52). Together, our study demonstrates that multilevel analysis is capable of defining gene regulation that not only reflects differences in EA and AsA populations, but also represents candidate pathways that may be the target of ancestry-specific therapies.

### Material and Methods

Genomic functional categories. The Variant Effect Predictor (VEP) tool available on the Ensembl genome browser 93 was used for annotation information to specify SNPs located within non-coding regions, including micro (mi) RNAs, long non-coding (Inc) RNAs, splice region variants, non-coding transcript exon variants, introns and intergenic regions. Regulatory regions include transcription factor binding sites (TFBS), promoters, enhancers, repressors, promoter flanking regions (PFRs) and open chromatin (OCRs). Coding regions were broken down further and include 5′UTRs, 3′UTRs, synonymous and nonsynonymous (missense and nonsense) mutations. The online resource tool HaploReg (version 4.153; was also used to identify DNA features, regulatory elements and assess regulatory potential.

Identification of SLE-associated SNPs and predicted genes. SLE Immunochip studies identified single nucleotide polymorphisms (SNPs) significantly associated with SLE in EA (6748 cases; 11,516 controls, p<1×10−6) (8). Because of the lower power of the East Asian Immunochip analysis reported in Sun et al. (6) (2485 cases and 3947 controls from Koreans (KR), Han Chinese (HC) and Malaysian Chinese (MC)), we identified 700 SNPs from 578 associated regions using a significance threshold of p<5×10−3). Because of the extensive linkage disequilibrium in the HLA region, SNPs in the region spanning chr6: 28014374-33683352 were omitted from the analysis. Asian validation SNPs were previously described (7,9). Expression quantitative trait loci (eQTLs) were then identified using GTEx version (8) (GTEXportal.org54) and the Blood eQTL browser database (13) and mapped to their associated eQTL expression genes (E-Genes). To find SNPs in enhancers and promoters, especially in intergenic regions, and their associated transcription factors and downstream target genes (T-Genes), we queried the atlas of Human Active Enhancers to interpret Regulatory variants (HACER) (15) and the GeneHancer database (14). To find structural SNPs in protein-coding genes (C-Genes), we queried the human Ensembl genome browser (GRCh38.p12) and dbSNP. Several additional databases were used to generate loss-of-function prediction scores, including SIFT4G55,56 and PolyPhen-257. All other SNPs were linked to the most proximal gene (P-Gene) or gene region as previously detailed (8). Predicted genes were examined as equal entities; no gene, regardless of provenance, was given more weight or importance over another type. For overlap studies, Venn diagrams were computed and visualized using InteractiVenn58. All predicted genes were divided into an EA, AsA or shared group depending on the ancestral designation of the original SLE-associated SNP. The random gene cohort was generated using the Random Gene Set Generator.

Differential gene expression analysis. For DEG analysis of E-MTAB-11191, affymetrix probes were annotated with custom BrainArray (BA) chip definition files (CDFs) (59) as previously described (60). Any probes with different Affymetrix and BA gene annotations were excluded. GCRMA-normalized expression values were variance corrected using local empirical Bayesian shrinkage before calculation of differentially expressed genes (DEGs) using the ebayes function in the BioConductor LIMMA package. P-values were adjusted for multiple hypothesis testing using the Benjamini-Hochberg False discovery rate (FDR). Significant Affymetrix and BA probes within each study were merged and filtered to retain probes with a pre-set FDR <0.2 which were considered statistically significant. This FDR threshold was employed to avoid falsely excluding genes of interest. This list was further filtered to retain only the most significant probe per gene in order to remove duplicate genes.

Functional gene set analysis. For both ancestral groups, predicted gene lists were examined using Biologically Informed Gene Clustering (BIG-C; version 4.4.). BIG-C is a custom functional clustering tool developed to annotate the biological meaning of large lists of genes. Genes are sorted into 54 categories based on their most likely biological function and/or cellular localization based on information from multiple online tools and databases including UniProtKB/Swiss-Prot, gene ontology (GO) Terms, MGI database, KEGG pathways61, NCBI, PubMed, and the Interactome, and has been previously described (62,63). I-Scope is a custom clustering tool used to identify immune infiltrates in large gene datasets, and has been described previously (64). Briefly, I-Scope was created through an iterative search of more than 17,000 genes identified in more than 50 microarray datasets. These genes were researched for immune cell specific expression in 30 hematopoietic sub-categories: T cells, regulatory T cells, activated T cells, anergic T cells, CD4 T cells, CD8 T cells, gamma-delta T cells, NK/NKT cells, T & B cells, B cells, activated B cells, T, B & myeloid, monocytes, monocytes & B cells, MHC Class II expressing cells, monocyte dendritic cells, dendritic cells, plasmacytoid dendritic cells, Langerhans cells, myeloid cells, plasma cells, erythrocytes, neutrophils, low density granulocytes, granulocytes, platelets, and all hematopoietic stem cells. Enrichment of GO Biological Processes (BP) using Enrichr65 and the Ingenuity Pathway Analysis (IPA) platform provided additional genetic pathway identification.

Network analysis and visualization. For SNP-predicted genes, only those encoding proteins included on the STRINGdb (version 1.3.2) platform were incorporated into protein-protein interaction (PPI) networks. To provide a quality control checkpoint, the significance of network connectivity was assessed, and we only moved forward with pathway analysis if the PPI enrichment p-value was <1×10−16 (lowest p-value, highest possible probability estimate). Visualization of protein-protein interaction and relationships between genes within datasets was done using Cytoscape (version 3.6.1) software. Briefly, STRINGdb generated networks were imported into Cytoscape and partitioned with MCODE via the clusterMaker2 (version 1.2.1) plugin.

Gene set variation analysis (GSVA). The GSVA (V1.25.0) software package for R/Bioconductor and has been described previously. Briefly, GSVA is a nonparametric, unsupervised method for estimating the variation of pre-defined gene sets in patient and control samples of microarray expression datasets. The input for the GSVA algorithm was a gene expression matrix of log 2 microarray of expression values and a collection of pre-defined gene signatures. Enrichment scores (GSVA scores) were calculated non-parametrically using a Kolmogorov-Smirnoff (KS)-like random walk statistic and a negative value for each gene set. GSVA gene signatures using official gene symbols are listed in Table S7). All interferon and cytokine signatures (core IFN, IFNB1, IFNA2, IFNW, IFNG and TNF) have been described previously. Metabolic signatures were based on literature mining and established IPA canonical pathways. Enrichment of each signature was examined in EA and AsA SLE patients and healthy control PBMCs from FDAPBMC1 for EA or GSE81622 for AsA. Differences between controls and SLE patient GSVA enrichment scores were determined using the Welch's t-test for unequal variances in Graphpad PRISM 8.0.

Regression models For all linear models, GSVA scores for cell type and/or pathway in each sample were used as input. Simple linear regression was performed in Graphpad PRISM 8.0.

### REFERENCES

1. Li, P. H. & Lau, C. S. Lupus in the far East: A modern epidemic. Int. J. Rheum. Dis. 20, 523-525 (2017).
2. Li, M. et al. Chinese SLE Treatment and Research group (CSTAR) registry: I. Major clinical characteristics of Chinese patients with systemic lupus erythematosus. Lupus 22, 1192-1199 (2013).
3. Yap, D. Y. H. & Chan, T. M. Lupus nephritis in Asia: Clinical features and management. Kidney Dis. 1, 100-109 (2015).
4. Lanata, C. M. et al. Genetic contributions to lupus nephritis in a multi-ethnic cohort of systemic lupus erythematous patients. PLOS ONE 13, e0199003 (2018).
5. Mok, M. Y. & Li, W. L. Do Asian patients have worse lupus?. Lupus 19, 1384-1390 (2010).
6. Sun, C. et al. High-density genotyping of immune-related loci identifies new SLE risk variants in individuals with Asian ancestry. Nat. Genet. 48, 323-330 (2016).
7. Lessard, C. J. et al. Identification of a systemic lupus erythematosus risk locus spanning ATG16L2, FCHSD2, and P2RY2 in Koreans. Arthritis Rheumatol. 68, 1197-1209 (2016).
8. Langefeld, C. D. et al. Transancestral mapping and genetic load in systemic lupus erythematosus. Nat. Commun. 8, 16021 (2017).
9. Morris, D. L. et al. Genome-wide association meta-analysis in Chinese and European individuals identifies ten new loci associated with systemic lupus erythematosus. Nat. Genet. 48, 940-946 (2016).
10. Bentham, J. et al. Genetic association analyses implicate aberrant regulation of innate and adaptive immunity genes in the pathogenesis of systemic lupus erythematosus. Nat. Genet. https://doi.org/10.1038/ng.3434 (2015).
11. Owen, K. A. et al. Analysis of trans-ancestral SLE risk loci identifies unique biologic networks and drug targets in African and European Ancestries. Am. J. Hum. Genet. 107, 864-881 (2020).
12. Kain, J. et al. Mendelian randomization and pathway analysis demonstrate shared genetic associations between lupus and coronary artery disease. Cell Rep. Med. 3, 100805 (2022).
13. Westra, H. J. et al. Systematic identification of trans eQTLs as putative drivers of known disease associations. Nat. Genet. 45, 1238-1243 (2013).
14. Fishilevich, S. et al. GeneHancer: Genome-wide integration of enhancers and target genes in GeneCards. Database (Oxford). 2017, bax028 (2017).
15. Wang, J. et al. HACER: An atlas of human active enhancers to interpret regulatory variants. Nucleic Acids Res. 47, D106-D112 (2019).
16. Kingsmore, K. M. et al. Altered expression of genes controlling metabolism characterizes the tissue response to immune injury in lupus. Sci. Rep. 11, 14789 (2021).
17. Cortes, A. & Brown, M. A. Promise and pitfalls of the Immunochip. Arthritis Res. Ther. 13, 101 (2011).
18. Li, P. et al. Glutathione peroxidase 4-regulated neutrophil ferroptosis induces systemic autoimmunity. Nat. Immunol. 22, 1107-1117 (2021).
19. Hänzelmann, S., Castelo, R. & Guinney, J. GSVA: Gene set variation analysis for microarray and RNA-Seq data. BMC Bioinform. 14, 7 (2013).
20. Lightfoot, Y. L., Blanco, L. P. & Kaplan, M. J. Metabolic abnormalities and oxidative stress in lupus. Curr. Opin. Rheumatol. 29, 442-449 (2017).
21. Andreoletti, G. et al. Transcriptomic analysis of immune cells in a multi-ethnic cohort of systemic lupus erythematosus patients identifies ethnicity- and disease-specific expression signatures. Commun. Biol. 4, 488 (2021).
22. Shah, D., Mahajan, N., Sah, S., Nath, S. K. & Paudyal, B. Oxidative stress and its biomarkers in systemic lupus erythematosus. J. Biomed. Sci. 21, 23 (2014).
23. Namas, R., Renauer, P., Ognenovski, M., Tsou, P. S. & Sawalha, A. H. Histone H2AX phosphorylation as a measure of DNA double-strand breaks and a marker of environmental stress and disease activity in lupus. Lupus Sci. Med. 3, e000148 (2016).
24. Zhang, H. et al. Anti-dsDNA antibodies bind to TLR4 and activate NLRP3 inflammasome in lupus monocytes/macrophages. J. Transl. Med. 14, 156 (2016).
25. Giles, B. M. & Boackle, S. A. Linking complement and anti-dsDNA antibodies in the pathogenesis of systemic lupus erythematosus. Immunol. Res. 55, 10 (2013).
26. Caielli, S. et al. Erythroid mitochondrial retention triggers myeloid-dependent type I interferon in human SLE. Cell 184, 4464-4479.e19 (2021).
27. Lewis, M. J. & Jawad, A. S. The effect of ethnicity and genetic ancestry on the epidemiology, clinical features and outcome of systemic lupus erythematosus. Rheumatology (Oxford) 56, 167-177 (2017).
28. Feldman, C. H. et al. Epidemiology and sociodemographics of systemic lupus erythematosus and lupus nephritis among US adults with Medicaid coverage, 2000-2004. Arthritis Rheum. 65, 753-763 (2013).
29. Robinson, E. K., Covarrubias, S. & Carpenter, S. The how and why of lncRNA function: An innate immune perspective. Biochimica et Biophysica Acta Gene Regulat. Mech. 1863, 194419 (2020).
30. Riege, K. et al. Massive effect on LncRNAs in human monocytes during fungal and bacterial infections and in response to vitamins A and D. Sci. Rep. 7, 40598 (2017).
31. Stein, N. et al. IFNG-AS1 enhances interferon gamma production in human natural killer cells. iScience 11, 466-473 (2019).
32. Dai, R. & Ahmed, S. A. microRNA, a new paradigm for understanding immunoregulation, inflammation, and autoimmune diseases. Transl. Res. 157, 163 (2011).
33. Tsai, C-Y. et al. Cross-talk between mitochondrial dysfunction-provoked oxidative stress and aberrant noncoding RNA expression in the pathogenesis and pathophysiology of SLE. Int. J. Mol. Sci. 20, 5183 (2019).
34. Tsai, C.-Y. et al. Aberrant non-coding RNA expression in patients with systemic lupus erythematosus: Consequences for immune dysfunctions and tissue damage. Biomolecules 10, 1-24 (2020).
35. Wang, X. et al. Long noncoding RNAs in the regulation of oxidative stress. Oxid. Med. Cell. Longev. 2019, U.S. Pat. No. 1,318,795 (2019).
36. Catalina, M. D. et al. Patient ancestry significantly contributes to molecular heterogeneity of systemic lupus erythematosus. JCI Insight. 5, e140380 (2020).
37. Wang, Y-F. et al. Identification of 38 novel loci for systemic lupus erythematosus and genetic heterogeneity between ancestral groups. Nat. Commun. 12, 772 (2021).
38. Griss, J. et al. B cells sustain inflammation and predict response to immune checkpoint blockade in human melanoma. Nat. Commun. 10, 4186 (2019).
39. Nossent, J. C., Becker-Merok, A., Rischmueller, M. & Lester, S. Susceptibility for lupus nephritis by low copy number of the FCGR3B gene is linked to increased levels of pathogenic autoantibodies. Autoimmune Dis. 2013, 750814 (2013).
40. Banchereau, R. et al. Personalized immunomonitoring uncovers molecular networks that stratify lupus patients. Cell 165, 551-565 (2016).
41. Menard, L. C. et al. B cells from African American lupus patients exhibit an activated phenotype. JCI Insight 1, e87310 (2016).
42. Cruz, C. S. D. & Kang, M.-J. Mitochondrial dysfunction and damage associated molecular patterns (DAMPs) in chronic inflammatory diseases. Mitochondrion 41, 37 (2018).
43. Buskiewicz, I. A. et al. Reactive oxygen species induce virus-independent MAVS oligomerization in systemic lupus erythematosus. Sci. Signal. 9, ra115 (2016).
44. Ratliff, B. B., Abdulmahdi, W., Pawar, R. & Wolin, M. S. Oxidant mechanisms in renal injury and disease. Antioxid. Redox Signal. 25, 119 (2016).
45. Jakes, R. W. et al. Systematic review of the epidemiology of systemic lupus erythematosus in the Asia-Pacific region: Prevalence, incidence, clinical features, and mortality. Arthritis Care Res. 64, 159-168 (2012).
46. Richman, I. B. et al. European genetic ancestry is associated with a decreased risk of lupus nephritis. Arthritis Rheum. 64, 3374-3382 (2012).
47. Honarpisheh, M., Köhler, P., Rauchhaupt, E. von & Lech, M. The involvement of microRNAs in modulation of innate and adaptive immunity in systemic lupus erythematosus and lupus nephritis. J. Immunol. Res. 2018, U.S. Pat. No. 4,126,106 (2018).
48. Zhang, C., Wang, H., Yin, L., Mao, Y. & Zhou, W. Immunometabolism in the pathogenesis of systemic lupus erythematosus. J. Transl. Autoimmun. 3, 100046 (2020).
49. Grayson, P. C. et al. Metabolic pathways and immunometabolism in rare kidney diseases. Ann. Rheum. Dis. 77, 1226 (2018).
50. Fernandez, D., Bonilla, E., Mirza, N., Niland, B. & Perl, A. Rapamycin reduces disease activity and normalizes T cell activation-induced calcium fluxing in patients with systemic lupus erythematosus. Arthritis Rheum. 54, 2983-2988 (2006).
51. Lai, Z.-W. et al. N-Acetylcysteine reduces disease activity by blocking mammalian target of rapamycin in T cells from systemic lupus erythematosus patients: A randomized, double-blind, placebo-controlled trial. Arthritis Rheum. 64, 2937-2946 (2012).
52. Liu, D. & Zhang, W. Pioglitazone attenuates lupus nephritis symptoms in mice by modulating miR-21-5p/TIMP3 Axis: The key role of the activation of peroxisome proliferator-activated receptor-γ. Inflammation 44, 1416-1425 (2021).
53. Ward, L. D. & Kellis, M. HaploReg v4: Systematic mining of putative causal variants, cell types, regulators and target genes for human complex traits and disease. Nucleic Acids Res. 44, D877-D881 (2016).
54. The Genotype-Tissue Expression (GTEx) project. https://www.ncbi.nlm.nih.gov/pmc/articles/PMC4010069/. (2013).
55. Vaser, R., Adusumalli, S., Leng, S. N., Sikic, M. & Ng, P. C. SIFT missense predictions for genomes. Nat. Protoc. 11, 1-9 (2016).
56. Sim, N. L. et al. SIFT web server: Predicting effects of amino acid substitutions on proteins. Nucleic Acids Res. 40, W452-7 (2012).
57. Adzhubei, I., Jordan, D. M. & Sunyaev, S. R. Predicting functional effect of human missense mutations using PolyPhen-2. Curr. Protoc. Hum. Genet. https://doi.org/10.1002/0471142905.hg0720s76 (2013).
58. Heberle, H., Meirelles, V. G., da Silva, F. R., Telles, G. P. & Minghim, R. InteractiVenn: A web-based tool for the analysis of sets through Venn diagrams. BMC Bioinform. 16, 169 (2015).
59. Dai, M. et al. Evolving gene/transcript definitions significantly alter the interpretation of GeneChip data. Nucleic Acids Res. 33, e175 (2005).
60. Labonte, A. C. et al. Identification of alterations in macrophage activation associated with disease activity in systemic lupus erythematosus. PLOS ONE 13, e0208132 (2018).
61. Kanehisa, M., Furumichi, M., Sato, Y., Kawashima, M. & Ishiguro-Watanabe, M. KEGG for taxonomy-based analysis of pathways and genomes. Nucleic Acids Res. 51, D587-D592 (2023).
62. Catalina, M. D., Bachali, P., Geraci, N. S., Grammer, A. C. & Lipsky, P. E. Gene expression analysis delineates the potential roles of multiple interferons in systemic lupus erythematosus. Commun. Biol. 2, 140 (2019).
63. Catalina, M. D., Owen, K. A., Labonte, A. C., Grammer, A. C. & Lipsky, P. E. The pathogenesis of systemic lupus erythematosus: Harnessing big data to understand the molecular basis of lupus. J. Autoimmun. https://doi.org/10.1016/j.jaut.2019.102359 (2019).
64. Ren, J. et al. Selective histone deacetylase 6 inhibition normalizes B cell activation and Germinal Center Formation in a model of systemic lupus erythematosus. Front. Immunol. 10, 2512 (2019).
65. Xie, Z. et al. Gene set knowledge discovery with Enrichr. Curr. Protoc. 1, e90 (2021).

TABLE 13
Immunochip SNPs and their location and ancestral, genomic, functional and discovery
method/source designation. Listed as: SNP, Chromosome:position, Ancestry, Genomic annotation,
Functional category, eQTL designation, Regulatory element;. The absence of any of these
seven items is indicated by a space (e.g., “; ,” or “, ,” or “, ;”).

rs10179521, 2:68316162, AsA, 3′UTR, Coding, , ; rs10194, X:155122975, AsA, 3′UTR, Coding,
eQTL, ; rs1048055, 20:1629416, AsA, 3′UTR, Coding, eQTL, ; rs1059702, X:154018741, AsA,
Missense, Coding, eQTL, ; rs10782546, 1:85251124, AsA, 3′UTR, Coding, eQTL, ; rs10936599,
3:169774313, AsA, synonomous, Coding, eQTL, ; rs11596235, 10:102631277, AsA, 3′UTR, Coding,
eQTL, ; rs1167827, 7:75533848, AsA, 3′UTR, Coding, eQTL, ; rs1169310, 12:121001630, AsA,
3′UTR, Coding, eQTL, ; rs11718350, 3:48319502, AsA, Missense, Coding, eQTL, ; rs11748,
11:403980, AsA, synonomous, Coding, eQTL, ; rs1217397, 1:113904943, AsA, 5′UTR, Coding,
eQTL, ; rs12619169, 2:102394624, AsA, Missense, Coding, , ; rs12795576, 11:119335120, AsA,
5′UTR, Coding, eQTL, ; rs13306575, 1:183563302, AsA, Missense, Coding, , ; rs138410054,
X:338627, AsA, synonomous, Coding, , ; rs1433770, 11:74719140, AsA, 5′UTR, Coding, eQTL, ;
rs146688502, X:1389431, AsA, synonomous, Coding, , ; rs1545620, 19:17192965, AsA, Missense,
Coding, eQTL, ; rs16959572, 16:57946398, AsA, 5′UTR, Coding, , ; rs1800385, 12:6018725, AsA,
Missense, Coding, , ; rs1872691, 16:50316299, AsA, 3′UTR, Coding, eQTL, ; rs2227902,
4:56930934, AsA, Missense, Coding, eQTL, ; rs2236259, 6:154157758, AsA, 3′UTR, Coding, , ;
rs2240188, 12:112965879, AsA, Missense, Coding, eQTL, ; rs2287717, 19:46755585, AsA,
synonomous, Coding, eQTL, ; rs2305115, 19:37344756, AsA, synonomous, Coding, eQTL, ;
rs249496, 5:102865886, AsA, 5′UTR, Coding, eQTL, ; rs2564097, 2:61050917, AsA, 3′UTR, Coding,
eQTL, ; rs26506, 5:96743698, AsA, Missense, Coding, eQTL, ; rs26722, 5:33963765, AsA, Missense,
Coding, , ; rs34193571, 5:81113707, AsA, Missense, Coding, , ; rs35211634, 11:59845386, AsA,
Missense, Coding, eQTL, ; rs3746522, 20:46051773, AsA, synonomous, Coding, eQTL, ; rs3747333,
X:5893491, AsA, Missense, Coding, , ; rs3767747, 1:198694973, AsA, 3′UTR, Coding, , ; rs3820632,
1:226603250, AsA, 3′UTR, Coding, eQTL, ; rs4359426, 16:57358821, AsA, Missense, Coding, eQTL,
; rs4723641, 7:37353647, AsA, 5′UTR, Coding, , ; rs4842838, 15:83913372, AsA, Missense, Coding,
eQTL, ; rs489574, 11:65775268, AsA, 3′UTR, Coding, eQTL, ; rs5744168, 1:223111858, AsA,
nonsense, Coding, eQTL, ; rs61743146, 5:110420735, AsA, nonsense, Coding, , ; rs61803003,
1:161623678, AsA, 3′UTR, Coding, eQTL, ; rs633924, 20:3659410, AsA, 3′UTR, Coding, eQTL, ;
rs6713162, 2:151640012, AsA, Missense, Coding, eQTL, ; rs697839, 1:226608274, AsA, 3′UTR,
Coding, eQTL, ; rs7097397, 10:48817351, AsA, Missense, Coding, eQTL, ; rs75175362,
19:46411670, AsA, Missense, Coding, eQTL, ; rs7783, 19:16518378, AsA, 3′UTR, Coding, eQTL, ;
rs7916697, 10:68232096, AsA, 5′UTR, Coding, eQTL, ; rs8065080, 17:3577153, AsA, Missense,
Coding, eQTL, ; rs808119, X:8536792, AsA, Missense, Coding, eQTL, ; rs8572, 14:22767315, AsA,
Missense, Coding, eQTL, ; rs885458, 1:167424398, AsA, 3′UTR, Coding, , ; rs1001487
4:140947361, AsA, intron, Non-coding, eQTL, ; rs10040412, 5:4417489, AsA, intergenic, Non-
coding, , ; rs10043548, 5:34536951, AsA, intergenic, Non-coding, eQTL, HACER/GeneHancer;
rs10049445, 3:40144932, AsA, intron, Non-coding, eQTL, ; rs10084421, 2:238413624, AsA,
intergenic, Non-coding, eQTL, ; rs1012053, 13:42079301, AsA, intron, Non-coding, eQTL, ;
rs10149918, 14:35163281, AsA, intron, Non-coding, , ; rs10180024, 2:61563929, AsA, intergenic,
Non-coding, eQTL, ; rs10185029, 2:190683501, AsA, intron, Non-coding, eQTL, ; rs10185928,
2:171634004, AsA, intergenic, Non-coding, eQTL, ; rs10189117, 2:33463583, AsA, intron, Non-
coding, , ; rs10280281, 7:16365684, AsA, intron, Non-coding, , ; rs1034796, 7:79474220, AsA,
intergenic, Non-coding, eQTL, ; rs1038390, 6:143505965, AsA, splice region variant, Non-coding,
eQTL, ; rs10411210, 19:33041394, AsA, intron, Non-coding, eQTL, HACER/GeneHancer;
rs10412569, 19:54986487, AsA, intron, Non-coding, eQTL, ; rs10422892, 19:55211473, AsA,
intergenic, Non-coding, eQTL, ; rs10451576, 2:206491609, AsA, intron, Non-coding, eQTL, ;
rs10473594, 5:22475991, AsA, intron, Non-coding, , ; rs10486304, 7:18351934, AsA, intron, Non-
coding, eQTL, ; rs10493830, 1:89757037, AsA, intron, Non-coding, , ; rs10504885, 8:89938825, AsA,
intron, Non-coding, , ; rs10506220, 12:42913602, AsA, intergenic, Non-coding, , ; rs10508470,
10:13979822, AsA, intron, Non-coding, eQTL, ; rs10512374, 9:107973283, AsA, intergenic, Non-
coding, , ; rs10514192, 18:75888292, AsA, intergenic, Non-coding, , ; rs10518784, 15:54829321,
AsA, intergenic, Non-coding, , ; rs10735599, 9:70588696, AsA, intron, Non-coding, eQTL, ;
rs10759236, 9:107448440, AsA, non coding transcript exon variant, Non-coding, , ; rs10771919,
12:9594018, AsA, intron, Non-coding, eQTL, ; rs1077989, 14:67509105, AsA, intron, Non-coding,
eQTL, ; rs10790077, 11:115471174, AsA, intron, Non-coding, eQTL, ; rs10795942, 10:12233537,
AsA, intron, Non-coding, eQTL, ; rs10803271, 1:14374333, AsA, intron, Non-coding, , ; rs10804839
3:170353995, AsA, intergenic, Non-coding, eQTL, ; rs10821699, 10:60197410, AsA, intron, Non-
coding, eQTL, ; rs10826721, 10:29883961, AsA, intergenic, Non-coding, , ; rs10831497,
11:96214246, AsA, intron, Non-coding, , ; rs10834134, 11:23551175, AsA, intergenic, Non-coding, , ;
rs10847864, 12:122842051, AsA, intron, Non-coding, eQTL, ; rs10860755, 12:101615828, AsA,
intron, Non-coding, , ; rs10863389, 1:206482950, AsA, intron, Non-coding, eQTL, ; rs10886880,
10:121254407, AsA, intergenic, Non-coding, eQTL, ; rs10937658, 4:5681084, AsA, intron, Non-
coding, , ; rs10967292, 9:26294089, AsA, intergenic, Non-coding, , ; rs11048030, 12:25437666, AsA,
intron, Non-coding, , ; rs11065976, 12:111609767, AsA, intergenic, Non-coding, eQTL, ; rs11071720,
15:63049797, AsA, intron, Non-coding, eQTL, ; rs11109229, 12:97814372, AsA, intergenic, Non-
coding, , ; rs11118092, 1:206483428, AsA, intron, Non-coding, eQTL, ; rs11128551, 3:11442381,
AsA, intron, Non-coding, , ; rs11132321, 4:185547838, AsA, intergenic, Non-coding, eQTL, ;
rs11150604, 16:31025699, AsA, intergenic, Non-coding, eQTL, HACER/GeneHancer; rs11221442,
11:128707729, AsA, intron, Non-coding, , ; rs11229889, 11:59252946, AsA, intergenic, Non-coding, ,
; rs11234164, 11:71557208, AsA, intergenic, Non-coding, eQTL, ; rs11235460, 11:70163614, AsA,
intron, Non-coding, eQTL, ; rs11244506, 10:125458167, AsA, intergenic, Non-coding, eQTL, ;
rs11264750, 1:157527370, AsA, intron, Non-coding, eQTL, ; rs11265489, 1:160807758, AsA, intron,
Non-coding, eQTL, ; rs11575587, 9:34656036, AsA, intron, Non-coding, , ; rs11585739,
1:230110153, AsA, intron, Non-coding, eQTL, ; rs11636932, 15:38561434, AsA, intron, Non-coding,
eQTL, ; rs11644322, 16:79005703, AsA, intron, Non-coding, , ; rs11650535, 17:77815179, AsA,
intergenic, Non-coding, , ; rs11676956, 2:185359937, AsA, intergenic, Non-coding, , ; rs11691193,
2:111096947, AsA, intron, Non-coding, eQTL, ; rs116986260, 16:75192544, AsA, non coding
transcript exon variant, Non-coding, eQTL, ; rs11727622, 4:130802636, AsA, intergenic, Non-coding,
, ; rs11753660, 6:125011030, AsA, intron, Non-coding, , ; rs11759658, 6:25359317, AsA, intron,
Non-coding, eQTL, ; rs11889341, 2:191079016, AsA, intron, Non-coding, , ; rs11910104,
21:42446059, AsA, intron, Non-coding, eQTL, ; rs11950355, 5:115270237, AsA, intron, Non-coding,
eQTL, ; rs12041565, 1:245609739, AsA, intron, Non-coding, eQTL, ; rs12045223, 1:5135322, AsA,
intergenic, Non-coding, , ; rs12068756, 1:233807282, AsA, intergenic, Non-coding, , ; rs12089677,
1:22356537, AsA, intergenic, Non-coding, eQTL, HACER/GeneHancer; rs12139150, 1:161500715,
AsA, intergenic, Non-coding, eQTL, ; rs12163856, 4:101826770, AsA, intron, Non-coding, eQTL, ;
rs12189864, 6:170376449, AsA, intron, Non-coding, eQTL, ; rs12235514, 9:114795706, AsA, intron,
Non-coding, , ; rs12247536, 10:12159852, AsA, intron, Non-coding, eQTL, ; rs12330287,
3:45170640, AsA, intergenic, Non-coding, eQTL, ; rs1233650, 17:34314911, AsA, intergenic, Non-
coding, , ; rs12355138, 10:58380949, AsA, intergenic, Non-coding, eQTL, ; rs12356376,
10:79302350, AsA, intron, Non-coding, , ; rs12439525, 15:74795064, AsA, intron, Non-coding,
eQTL, ; rs12444713, 16:31366914, AsA, intron, Non-coding, eQTL, ; rs12480543, 20:361302, AsA,
intergenic, Non-coding, , ; rs12481660, 20:40418676, AsA, intergenic, Non-coding, eQTL, ;
rs12525855, 6:167066525, AsA, intron, Non-coding, eQTL, ; rs12529149, 6:19321070, AsA,
intergenic, Non-coding, eQTL, ; rs1253687, 14:51914526, AsA, intron, Non-coding, , ; rs12548516,
8:12844415, AsA, intergenic, Non-coding, eQTL, ; rs12551314, 9:93617515, AsA, intron, Non-
coding, eQTL, ; rs12564478, 1:101198024, AsA, intergenic, Non-coding, , ; rs12575600*,
11:128454974, AsA, intergenic, Non-coding, , ; rs12576753, 11:128434246, AsA, intergenic, Non-
coding, , ; rs12603019, 17:16948308, AsA, intron, Non-coding, , ; rs12642398, 4:88402062, AsA,
intron, Non-coding, eQTL, ; rs1266574, 11:95766271, AsA, intergenic, Non-coding, eQTL, ;
rs12666501, 7:37386191, AsA, intron, Non-coding, eQTL, ; rs12744299, 1:160422364, AsA, intron,
Non-coding, eQTL, ; rs12869389, 13:42499323, AsA, intergenic, Non-coding, eQTL,
HACER/GeneHancer; rs13005185, 2:206439865, AsA, intergenic, Non-coding, eQTL, ; rs13015447,
2:166521468, AsA, intron, Non-coding, , ; rs13020141, 2:100037380, AsA, intron, Non-coding, , ;
rs13097583, 3:95640443, AsA, intergenic, Non-coding, , ; rs1315128, 14:33369154, AsA, intron,
Non-coding, , ; rs13176209, 5:96725995, AsA, intron, Non-coding, eQTL, ; rs13195040, 6:27446145,
AsA, intergenic, Non-coding, eQTL, ; rs13219796, 6:89947302, AsA, intron, Non-coding, , ;
rs13240375, 7:123806334, AsA, intergenic, Non-coding, eQTL, ; rs13403657, 2:241004033, AsA,
intron, Non-coding, , ; rs1340589, 1:67458708, AsA, intergenic, Non-coding, eQTL, ; rs13425999*,
2:33477136, AsA, intron, Non-coding, , ; rs13433939, 3:151990625, AsA, intergenic, Non-coding, , ;
rs1345389, 16:52413502, AsA, intergenic, Non-coding, , ; rs1350617, 4:117805467, AsA, intergenic,
Non-coding, eQTL, ; rs1363556, 5:145526893, AsA, intron, Non-coding, eQTL, ; rs1369320,
11:38382098, AsA, intergenic, Non-coding, , ; rs1372942, 18:30470231, AsA, intergenic, Non-coding,
, ; rs1405209, 9:99823263, AsA, intron, Non-coding, eQTL, ; rs1419559, 7:125933992, AsA,
intergenic, Non-coding, , ; rs1432079, 18:72581758, AsA, intron, Non-coding, , ; rs146535458,
X:2726261, AsA, splice region variant, Non-coding, , ; rs1465876, 2:4849337, AsA, intergenic, Non-
coding, , ; rs1485693, 7:68990513, AsA, intergenic, Non-coding, , ; rs1526602, 17:40721620, AsA,
non coding transcript exon variant, Non-coding, , ; rs1533298, 2:110858727, AsA, intron, Non-coding,
, ; rs1535, 11:61830500, AsA, intron, Non-coding, , ; rs1546913*, 20:57578979, AsA, intergenic,
Non-coding, , ; rs1551845, 12:78921018, AsA, intron, Non-coding, , ; rs1566028, 19:18147788, AsA,
intron, Non-coding, eQTL, ; rs1575335, 6:19095231, AsA, intergenic, Non-coding, eQTL, ;
rs1576168, 13:26928142, AsA, intergenic, Non-coding, eQTL, ; rs1599552, 18:45337821, AsA,
intron, Non-coding, , ; rs1600184, 5:35983479, AsA, intron, Non-coding, eQTL, ; rs1635239,
X:3314658, AsA, intron, Non-coding, eQTL, ; rs167392, 9:121334389, AsA, intergenic, Non-coding,
eQTL, ; rs16885587, 8:36936940, AsA, intergenic, Non-coding, , ; rs16897274, 5:67996054, AsA,
intergenic, Non-coding, , ; rs16923431, 8:58396199, AsA, intergenic, Non-coding, , ; rs16951783,
16:22829534, AsA, intron, Non-coding, eQTL, ; rs16963815, 16:61683457, AsA, intron, Non-coding,
, ; rs16971851, 15:33520308, AsA, intron, Non-coding, , ; rs16993330, 22:33947708, AsA, intergenic,
Non-coding, , ; rs17003642, 22:43308186, AsA, intron, Non-coding, , ; rs17016070, 2:128277509,
AsA, intron, Non-coding, eQTL, ; rs17037388, 1:11797979, AsA, intron, Non-coding, eQTL, ;
rs17040590, 2:78614652, AsA, intergenic, Non-coding, , ; rs17044912, 3:6445940, AsA, intergenic,
Non-coding, , ; rs170551, 1:7895834, AsA, intergenic, Non-coding, eQTL, ; rs17075364, 8:5959664,
AsA, intergenic, Non-coding, eQTL, ; rs17077968, 6:116448435, AsA, intron, Non-coding, eQTL, ;
rs17081657, 18:69813498, AsA, intron, Non-coding, , ; rs17082777, 4:62495609, AsA, intergenic,
Non-coding, , ; rs17089390, 18:74559622, AsA, intron, Non-coding, , ; rs17111124, 14:80821467,
AsA, intron, Non-coding, , ; rs17112138, 12:73207684, AsA, non coding transcript exon variant, Non-
coding, , ; rs17147106, 7:76585480, AsA, intron, Non-coding, eQTL, ; rs17152902, 8:11134361, AsA,
intron, Non-coding, eQTL, ; rs17161411, 7:98561594, AsA, intergenic, Non-coding, eQTL,
HACER/GeneHancer; rs17307029, 21:15345025, AsA, intergenic, Non-coding, , ; rs17321800,
9:83588127, AsA, intergenic, Non-coding, eQTL, ; rs1735937, 21:21706019, AsA, intergenic, Non-
coding, , ; rs17385169, 1:156014078, AsA, intron, Non-coding, eQTL, ; rs175100, 11:61041608, AsA,
intergenic, Non-coding, , ; rs17519442, 7:11632507, AsA, intron, Non-coding, , ; rs1754244,
10:30464782, AsA, intergenic, Non-coding, , ; rs17643558, 3:43376300, AsA, intron, Non-coding,
eQTL, ; rs1768198, 3:39474994, AsA, intron, Non-coding, eQTL, ; rs1769299, 1:53120051, AsA,
intron, Non-coding, eQTL, ; rs17703668, 13:59268925, AsA, intergenic, Non-coding, , ; rs17777179,
13:77744254, AsA, intron, Non-coding, eQTL, ; rs17800858, 12:1171224, AsA, intron, Non-coding, ,
; rs17806132, 2:189843561, AsA, intron, Non-coding, eQTL, ; rs17836364, 19:54336316, AsA,
intron, Non-coding, , ; rs17881940, 17:42324649, AsA, intron, Non-coding, eQTL, ; rs1795306
3:105286009, AsA, intergenic, Non-coding, , ; rs179570, 19:35760425, AsA, intron, Non-coding,
eQTL, ; rs1807017, 5:60831897, AsA, intron, Non-coding, eQTL, ; rs1811340, 5:76380833, AsA,
intergenic, Non-coding, , ; rs1812389, 1:97534131, AsA, intron, Non-coding, , ; rs182320,
6:129752146, AsA, intergenic, Non-coding, , ; rs1823373, 8:75744710, AsA, intergenic, Non-coding,
eQTL, ; rs1861757, 16:50718904, AsA, intron, Non-coding, eQTL, ; rs1862028, 12:105887577, AsA,
intergenic, Non-coding, eQTL, ; rs1865761, 18:69872156, AsA, intron, Non-coding, eQTL, ;
rs1888822, 6:35215372, AsA, intron, Non-coding, eQTL, HACER/GeneHancer; rs1899878,
13:21618965, AsA, intergenic, Non-coding, , ; rs1927005, X:92994625, AsA, intergenic, Non-coding,
, ; rs1949453, 2:181046047, AsA, intron, Non-coding, eQTL, ; rs1981458, 15:90873375, AsA, intron,
Non-coding, , ; rs198806, 6:26133388, AsA, intron, Non-coding, eQTL, ; rs1993007, 14:80533369,
AsA, intron, Non-coding, , ; rs1999080, 13:114221580, AsA, intron, Non-coding, eQTL,
HACER/GeneHancer; rs2003422, 8:11445628, AsA, intron, Non-coding, eQTL, ; rs2014519,
21:20818971, AsA, intergenic, Non-coding, , ; rs2023051, 6:162130067, AsA, intron, Non-coding, , ;
rs2037054, 12:101250739, AsA, intergenic, Non-coding, eQTL, ; rs2039124, 9:117671239, AsA,
intergenic, Non-coding, eQTL, ; rs2065333, 21:42459636, AsA, intergenic, Non-coding, eQTL, ;
rs2066190, 1:236876158, AsA, intron, Non-coding, eQTL, ; rs21140, 20:14872404, AsA, intron, Non-
coding, , ; rs2159272, 3:45788503, AsA, intron, Non-coding, eQTL, ; rs218311, 2:171201354, AsA,
intron, Non-coding, eQTL, ; rs2188355, 16:23856455, AsA, intron, Non-coding, eQTL,
HACER/GeneHancer; rs2205960*, 1:173222336, AsA, intergenic, Non-coding, , ; rs221899,
14:71138551, AsA, intergenic, Non-coding, eQTL, ; rs2222996, 21:39657904, AsA, intron, Non-
coding, eQTL, ; rs2236949, 3:50372610, AsA, intron, Non-coding, eQTL, ; rs223881*, 16:57352654,
AsA, intergenic, Non-coding, eQTL, ; rs2243263, 5:132677607, AsA, intron, Non-coding, eQTL, ;
rs2255852, 1:160957255, AsA, intergenic, Non-coding, eQTL, HACER/GeneHancer; rs2267546,
12:4636147, AsA, intron, Non-coding, eQTL, ; rs2269239, 1:63643688, AsA, intron, Non-coding,
eQTL, ; rs2269848, 19:1076064, AsA, intron, Non-coding, eQTL, ; rs2276122, 11:118094815, AsA,
splice region variant, Non-coding, eQTL, ; rs2285110, 22:37232105, AsA, intron, Non-coding, eQTL,
; rs228614, 4:102657480, AsA, intron, Non-coding, eQTL, ; rs2286439, 17:34272341, AsA,
intergenic, Non-coding, , ; rs2295591, 6:136845333, AsA, intron, Non-coding, , ; rs2300603,
14:75539214, AsA, intron, Non-coding, eQTL, ; rs232771, 1:58626087, AsA, intergenic, Non-coding,
eQTL, ; rs2330523, 7:53475580, AsA, intergenic, Non-coding, , ; rs2344173, 18:73819381, AsA,
intergenic, Non-coding, , ; rs2393372, 12:117980886, AsA, intergenic, Non-coding, , ; rs2404581,
12:40246099, AsA, intron, Non-coding, , ; rs2405523, 1:224724232, AsA, intron, Non-coding, eQTL,
; rs2456449*, 8:127180736, AsA, intron, Non-coding, , ; rs2479406, 1:55036999, AsA, intergenic,
Non-coding, eQTL, ; rs2488326, 10:32918569, AsA, intron, Non-coding, eQTL, ; rs2498644,
6:139048457, AsA, intergenic, Non-coding, eQTL, ; rs252813, 5:107398325, AsA, intron, Non-
coding, , ; rs2559658, 10:77985511, AsA, intron, Non-coding, eQTL, ; rs2569016, 5:103267431, AsA,
intron, Non-coding, eQTL, ; rs2583523, 4:115007036, AsA, intron, Non-coding, eQTL, ; rs266433*,
5:72875888, AsA, intron, Non-coding, eQTL, ; rs2685723, 2:81939591, AsA, intergenic, Non-coding,
, ; rs268706, 5:33405491, AsA, intergenic, Non-coding, eQTL, ; rs2693889, 2:6114620, AsA,
intergenic, Non-coding, , ; rs2704517, 10:69762122, AsA, intergenic, Non-coding, eQTL, ; rs2705635,
2:81764462, AsA, intergenic, Non-coding, , ; rs2706682, 17:67558173, AsA, intron, Non-coding, , ;
rs2728538, X:154796890, AsA, intron, Non-coding, eQTL, ; rs2735839, 19:50861367, AsA,
intergenic, Non-coding, , ; rs2754244, 6:87671503, AsA, intergenic, Non-coding, eQTL, ; rs2754986,
X:92424130, AsA, intron, Non-coding, , ; rs2819376, 1:201962091, AsA, intron, Non-coding, eQTL, ;
rs28376696, 12:123365316, AsA, intron, Non-coding, eQTL, HACER/GeneHancer; rs2853676,
5:1288432, AsA, intron, Non-coding, eQTL, ; rs2876900, 7:50856998, AsA, intergenic, Non-coding, ,
; rs2883927, 1:240444999, AsA, intron, Non-coding, , ; rs2887945, 3:27794436, AsA, intergenic,
Non-coding, , ; rs2889962, 9:84747124, AsA, intron, Non-coding, eQTL, ; rs2968222, 5:57632265,
AsA, intergenic, Non-coding, , ; rs3007738, 1:241557243, AsA, intron, Non-coding, eQTL, ;
rs304723, 19:43598186, AsA, intron, Non-coding, eQTL, ; rs3110697, 7:45915430, AsA, intron, Non-
coding, eQTL, ; rs3116521, 2:203916118, AsA, intergenic, Non-coding, eQTL, ; rs3125002,
9:136510909, AsA, intron, Non-coding, eQTL, ; rs3134883, 10:6058762, AsA, intron, Non-coding, , ;
rs3135695, 7:74236235, AsA, intron, Non-coding, eQTL, ; rs336286, 7:35260201, AsA, intergenic,
Non-coding, eQTL, ; rs337514, 12:62858411, AsA, intron, Non-coding, , ; rs35099199, 20:49791466,
AsA, intergenic, Non-coding, , ; rs35439826, 3:27797796, AsA, non coding transcript exon variant,
Non-coding, eQTL, ; rs35481031, 3:50997194, AsA, intron, Non-coding, eQTL, ; rs35741664,
16:11370918, AsA, intron, Non-coding, eQTL, ; rs360183, 14:46633274, AsA, intergenic, Non-
coding, , ; rs370971, 20:43742455, AsA, intergenic, Non-coding, , ; rs3759095, 12:56104154, AsA,
intron, Non-coding, , ; rs3761527, X:153903170, AsA, Intron, Non-coding, eQTL, ; rs3767502
1:201053970, AsA, intron, Non-coding, eQTL, ; rs3775536, 4:47093696, AsA, intron, Non-coding, , ;
rs3785794, 17:7102596, AsA, intron, Non-coding, , ; rs3789602, 1:113797129, AsA, intron, Non-
coding, , ; rs3795985, 2:218400673, AsA, Intron, Non-coding, eQTL, ; rs3796531, 4:56984680, AsA,
intron, Non-coding, eQTL, ; rs3885668, 2:10038352, AsA, intergenic, Non-coding, eQTL, ;
rs3905634, 10:11347401, AsA, intergenic, Non-coding, , ; rs399867, 7:159117938, AsA, intron, Non-
coding, eQTL, ; rs4075359, 8:9630303, AsA, intron, Non-coding, eQTL, ; rs4130885, 1:73109392,
AsA, intergenic, Non-coding, eQTL, ; rs413756, 13:110491408, AsA, intron, Non-coding, , ;
rs424012, 8:108142424, AsA, intergenic, Non-coding, eQTL, ; rs4273807, 7:51327163, AsA,
intergenic, Non-coding, eQTL, HACER/GeneHancer; rs4294053, 7:156058930, AsA, intergenic, Non-
coding, , ; rs4312730, 4:48180416, AsA, intron, Non-coding, eQTL, ; rs4323356, 6:451373, AsA,
intergenic, Non-coding, , ; rs433875, 20:44631646, AsA, intron, Non-coding, , ; rs4375232,
1:215097060, AsA, intron, Non-coding, eQTL, ; rs4395027, 15:93110051, AsA, intergenic, Non-
coding, , ; rs4440829, 1:4020720, AsA, intergenic, Non-coding, , ; rs4440941, 10:53154667, AsA,
intergenic, Non-coding, , ; rs4444271, 14:104695125, AsA, intron, Non-coding, eQTL, ; rs4499095,
12:124900968, AsA, intergenic, Non-coding, eQTL, ; rs4534498, 10:132146918, AsA, intron, Non-
coding, eQTL, ; rs455030, 7:159225515, AsA, intergenic, Non-coding, , ; rs455247, 6:111346596,
AsA, intron, Non-coding, eQTL, ; rs4558091, 10:49113873, AsA, intron, Non-coding, eQTL, ;
rs4621545, 5:79777389, AsA, intron, Non-coding, eQTL, ; rs4648892*, 1:23393692, AsA, intron,
Non-coding, eQTL, ; rs4671642, 2:65179343, AsA, intergenic, Non-coding, , ; rs4678000*
3:122171888, AsA, intergenic, Non-coding, eQTL, ; rs4680870, 3:27789604, AsA, intergenic, Non-
coding, , ; rs4682953, 3:42184459, AsA, intron, Non-coding, eQTL, ; rs4690055*, 4:2746936, AsA,
intron, Non-coding, eQTL, ; rs4728142, 7:128933913, AsA, intergenic, Non-coding, eQTL,
HACER/GeneHancer; rs4731532*, 7:128932712, AsA, intergenic, Non-coding, eQTL,
HACER/GeneHancer; rs4737532, 8:59025084, AsA, intron, Non-coding, , ; rs4746554, 10:66200705,
AsA, intron, Non-coding, , ; rs4750065, 10:6187726, AsA, intron, Non-coding, , ; rs4755422,
11:35662336, AsA, intergenic, Non-coding, eQTL, ; rs4755450, 11:36342025, AsA, intron, Non-
coding, eQTL, ; rs4756119, 11:34234830, AsA, intron, Non-coding, , ; rs4760497, 12:92789490, AsA,
intron, Non-coding, , ; rs4760593, 12:128802675, AsA, intron, Non-coding, eQTL, ; rs4766489,
12:109868540, AsA, intron, Non-coding, eQTL, ; rs4785326, 16:49581676, AsA, intron, Non-coding,
, ; rs4821116, 22:21619030, AsA, intron, Non-coding, eQTL, ; rs4833808, 4:122071344, AsA,
intergenic, Non-coding, , ; rs4840565, 8:11488036, AsA, intron, Non-coding, eQTL, ; rs4870268,
6:154151192, AsA, intron, Non-coding, , ; rs487642, 6:52953255, AsA, intron, Non-coding, eQTL, ;
rs4910170, 11:10691885, AsA, intron, Non-coding, eQTL, ; rs4930043, 11:2168753, AsA, intron,
Non-coding, eQTL, ; rs4939486, 11:61016637, AsA, intron, Non-coding, , ; rs495558, 6:70277497,
AsA, intron, Non-coding, eQTL, ; rs4957300, 5:40463637, AsA, non coding transcript exon variant,
Non-coding, eQTL, ; rs4963768, 12:24501342, AsA, intron, Non-coding, , ; rs4977520, 9:18655963,
AsA, intron, Non-coding, eQTL, ; rs498679, 6:106122200, AsA, intron, Non-coding, , ; rs5020219,
4:73170449, AsA, intron, Non-coding, eQTL, ; rs512535, 2:21044910, AsA, intergenic, Non-coding,
eQTL, ; rs515754, 18:79586682, AsA, non coding transcript exon variant, Non-coding, , ; rs515818,
3:183134639, AsA, intron, Non-coding, eQTL, ; rs530389, 3:119606705, AsA, intron, Non-coding,
eQTL, ; rs550640, X:123394952, AsA, intron, Non-coding, , ; rs55780475, X:89275066, AsA,
intergenic, Non-coding, , ; rs55859785, 6:106162903, AsA, intron, Non-coding, , ; rs566412,
7:154743801, AsA, intron, Non-coding, , ; rs57310779, 2:100264155, AsA, intergenic, Non-coding, , ;
rs57348955, 16:31174561, AsA, intergenic, Non-coding, eQTL, HACER/GeneHancer; rs57500105,
14:87911680, AsA, intron, Non-coding, eQTL, ; rs5751621, 22:23293187, AsA, intron, Non-coding,
eQTL, ; rs5753220, 22:30590363, AsA, intron, Non-coding, eQTL, ; rs57585717, 7:28109636, AsA,
intron, Non-coding, eQTL, ; rs5763688, 22:29997514, AsA, intron, Non-coding, eQTL, ; rs587862,
11:128725278, AsA, intron, Non-coding, , ; rs5920818, X:100314162, AsA, intron, Non-coding,
eQTL, ; rs5927375, X:35235002, AsA, intergenic, Non-coding, eQTL, ; rs5973024, X:29799493,
AsA, intron, Non-coding, , ; rs598786, 2:140297511, AsA, intron, Non-coding, , ; rs601356,
1:207579205, AsA, intron, Non-coding, eQTL, ; rs606076, 2:134536511, AsA, intron, Non-coding,
eQTL, ; rs6062501, 20:63710579, AsA, intron, Non-coding, eQTL, ; rs6072081, 20:40632414, AsA,
intergenic, Non-coding, , ; rs6074046, 20:46156292, AsA, intergenic, Non-coding, eQTL, ; rs6074973,
20:1665745, AsA, intron, Non-coding, , ; rs6094661, 20:47325548, AsA, intron, Non-coding, , ;
rs61150131, 11:118881469, AsA, intergenic, Non-coding, , ; rs61334194, 2:33477419, AsA, intron,
Non-coding, eQTL, ; rs613791, 11:118893342, AsA, intron, Non-coding, eQTL, ; rs61616683,
22:39359768, AsA, intron, Non-coding, eQTL, ; rs62266700, 3:119396685, AsA, intron, Non-coding,
eQTL, ; rs630044, 5:131922685, AsA, intron, Non-coding, eQTL, ; rs6437119, 2:157898812, AsA,
intron, Non-coding, , ; rs6452345, 5:26568134, AsA, intergenic, Non-coding, , ; rs6461513,
7:20724284, AsA, intron, Non-coding, , ; rs6480640, 10:72770449, AsA, intron, Non-coding, eQTL, ;
rs6499640, 16:53735765, AsA, intron, Non-coding, eQTL, ; rs6507823, 18:48186667, AsA, intron,
Non-coding, , ; rs6517385, 21:36861972, AsA, intron, Non-coding, eQTL, ; rs652944, 12:9944193,
AsA, intergenic, Non-coding, eQTL, ; rs6593993, 1:204093864, AsA, intron, Non-coding, , ;
rs6642096, X:3047125, AsA, intron, Non-coding, , ; rs6656609, 1:114002809, AsA, intron, Non-
coding, eQTL, ; rs6675994, 1:42860418, AsA, intergenic, Non-coding, eQTL, ; rs6690453,
1:242746191, AsA, intergenic, Non-coding, , ; rs671188, 11:102958726, AsA, intergenic, Non-coding,
, ; rs6731993, 2:65414963, AsA, intron, Non-coding, , ; rs6732469, 2:128249887, AsA, intron, Non-
coding, eQTL, ; rs6733999, 2:105642841, AsA, intergenic, Non-coding, , ; rs6741204, 2:140216589,
AsA, intergenic, Non-coding, , ; rs6743559, 2:4259627, AsA, intergenic, Non-coding, , ; rs6806528,
3:69203748, AsA, intron, Non-coding, eQTL, ; rs68171887, 2:184526158, AsA, intergenic, Non-
coding, , ; rs6824845, 4:164048536, AsA, intron, Non-coding, , ; rs6842303, 4:17852432, AsA, intron,
Non-coding, eQTL, ; rs6861906, 5:37075834, AsA, intergenic, Non-coding, eQTL, ; rs687670,
2:135983330, AsA, intron, Non-coding, eQTL, ; rs6876997, 5:134142020, AsA, intron, Non-coding,
eQTL, ; rs6898651, 5:159086823, AsA, intron, Non-coding, eQTL, ; rs6910968, 6:27936403, AsA,
intergenic, Non-coding, eQTL, ; rs6916301, 6:27074479, AsA, intergenic, Non-coding, eQTL,
HACER/GeneHancer; rs6934794, 6:27551566, AsA, intergenic, Non-coding, eQTL, ; rs6940226,
6:124520025, AsA, intron, Non-coding, , ; rs6964720, 7:75551049, AsA, intron, Non-coding, eQTL, ;
rs6984782, 8:56223330, AsA, intergenic, Non-coding, eQTL, ; rs6987649, 8:42424116, AsA, intron,
Non-coding, eQTL, ; rs7005823, 8:25398068, AsA, intron, Non-coding, eQTL, ; rs7023146,
9:5040163, AsA, intron, Non-coding, eQTL, ; rs7024096, 9:22399694, AsA, intergenic, Non-coding,
eQTL, ; rs7037277, 9:21772065, AsA, intergenic, Non-coding, , ; rs704409, 3:64261153, AsA, intron,
Non-coding, , ; rs7062732, X:7680936, AsA, intron, Non-coding, , ; rs70692, 6:36736441, AsA,
intergenic, Non-coding, eQTL, ; rs7108178, 11:75833253, AsA, intron, Non-coding, eQTL, ;
rs713743, 22:27292374, AsA, intergenic, Non-coding, , ; rs7184802, 16:50322085, AsA, intron, Non-
coding, eQTL, ; rs719927, 11:91910941, AsA, intergenic, Non-coding, , ; rs7218952, 17:27869146,
AsA, intron, Non-coding, eQTL, ; rs7224733, 17:83046894, AsA, intron, Non-coding, eQTL, ;
rs7225332, 17:5821921, AsA, intron, Non-coding, eQTL, ; rs723586, 16:11004522, AsA, intron, Non-
coding, eQTL, ; rs7275515, 21:44275860, AsA, intergenic, Non-coding, eQTL, ; rs72758110,
9:120776068, AsA, intron, Non-coding, eQTL, ; rs72928026, 6:90252386, AsA, intron, Non-coding, ,
; rs72974161, 2:136044886, AsA, intergenic, Non-coding, eQTL, ; rs7300860, 12:111316793, AsA,
intron, Non-coding, eQTL, ; rs7306706, 12:6106468, AsA, intron, Non-coding, eQTL, ; rs7321872,
13:37672470, AsA, intron, Non-coding, eQTL, ; rs73296396, 20:44557843, AsA, intron, Non-coding,
, ; rs73510898, 19:10305768, AsA, intron, Non-coding, eQTL, ; rs73800142, 6:159038898, AsA,
intron, Non-coding, eQTL, ; rs7384058, 7:129811104, AsA, intergenic, Non-coding, eQTL, ;
rs74556031, 6:20728142, AsA, intron, Non-coding, , ; rs746098, 2:52711977, AsA, intergenic, Non-
coding, , ; rs752280, 2:173688707, AsA, intergenic, Non-coding, , ; rs7536191, 1:152811084, AsA,
intergenic, Non-coding, eQTL, ; rs7568275*, 2:191101726, AsA, intron, Non-coding, eQTL, ;
rs7569284, 2:113045986, AsA, intron, Non-coding, eQTL, ; rs757725, 7:149812893, AsA, intron,
Non-coding, eQTL, ; rs7579944, 2:30222160, AsA, intergenic, Non-coding, , ; rs75829476,
1:192349154, AsA, intron, Non-coding, , ; rs7588196, 2:43133823, AsA, intergenic, Non-coding, , ;
rs7607205, 2:210624379, AsA, intron, Non-coding, eQTL, ; rs7629068, 3:60234758, AsA, intron,
Non-coding, , ; rs7653027, 3:16978365, AsA, intron, Non-coding, eQTL, ; rs7678588, 4:117742166,
AsA, intergenic, Non-coding, eQTL, ; rs7699654, 4:10711145, AsA, intergenic, Non-coding, , ;
rs7725218, 5:1282299, AsA, intron, Non-coding, , ; rs7726414*, 5:134096143, AsA, intergenic, Non-
coding, eQTL, HACER/GeneHancer; rs77278484, 9:122997743, AsA, intron, Non-coding, , ;
rs77279327, 6:127913847, AsA, intron, Non-coding, , ; rs7740927, 6:146677098, AsA, intron, Non-
coding, , ; rs77552606, 20:63679776, AsA, intron, Non-coding, eQTL, ; rs7761159, 6:167106419,
AsA, intron, Non-coding, eQTL, ; rs7779475, 7:149236851, AsA, intergenic, Non-coding, eQTL, ;
rs78146216, 11:118887205, AsA, intron, Non-coding, , ; rs7851766, 9:653505, AsA, intron, Non-
coding, eQTL, ; rs7858600, 9:102764080, AsA, intergenic, Non-coding, , ; rs7928668, 11:117929208,
AsA, intron, Non-coding, eQTL, ; rs79490407, 5:142086559, AsA, intergenic, Non-coding, eQTL, ;
rs7965927, 12:118571553, AsA, intergenic, Non-coding, , ; rs7977617, 12:104354059, AsA,
intergenic, Non-coding, eQTL, ; rs798000, 1:116738074, AsA, intergenic, Non-coding, eQTL, ;
rs7993355, 13:66749396, AsA, intron, Non-coding, , ; rs8005252, 14:35356230, AsA, intergenic,
Non-coding, eQTL, ; rs80120393, 1:198689353, AsA, intron, Non-coding, , ; rs8012888,
14:106218971, AsA, intergenic, Non-coding, , ; rs803125, 17:40581127, AsA, intergenic, Non-coding,
eQTL, HACER/GeneHancer; rs8031532, 15:88815098, AsA, intron, Non-coding, , ; rs8042800,
15:59558601, AsA, intergenic, Non-coding, , ; rs8060368, 16:27401093, AsA, intergenic, Non-coding,
eQTL, ; rs8064192, 16:55132009, AsA, intergenic, Non-coding, , ; rs8080957, 17:80608893, AsA,
intron, Non-coding, eQTL, ; rs814586, 5:10531886, AsA, intergenic, Non-coding, , ; rs876109,
1:24946797, AsA, intron, Non-coding, eQTL, ; rs884708, 13:111362756, AsA, intergenic, Non-
coding, eQTL, ; rs888989, 5:151051469, AsA, intron, Non-coding, eQTL, ; rs898329, 10:60287524,
AsA, intron, Non-coding, eQTL, ; rs902169, 12:124098900, AsA, intron, Non-coding, eQTL, ;
rs908858, 1:221709883, AsA, intron, Non-coding, , ; rs9291304, 4:47127439, AsA, intron, Non-
coding, eQTL, ; rs9291474, 4:10969187, AsA, intergenic, Non-coding, eQTL, ; rs929192,
7:89405629, AsA, intergenic, Non-coding, , ; rs9367646*, 6:55601574, AsA, intergenic, Non-coding,
eQTL, ; rs9381707, 6:48949646, AsA, intergenic, Non-coding, eQTL, ; rs9385445, 6:127994206,
AsA, intron, Non-coding, , ; rs9405680, 6:449281, AsA, intergenic, Non-coding, , ; rs9465874,
6:20737123, AsA, intron, Non-coding, , ; rs9469857, 6:34752016, AsA, intergenic, Non-coding,
eQTL, HACER/GeneHancer; rs9494894, 6:137907383, AsA, intergenic, Non-coding, eQTL, ;
rs9516021, 13:92226403, AsA, intron, Non-coding, , ; rs9534986, 13:48193196, AsA, intergenic,
Non-coding, , ; rs9581788, 13:26934001, AsA, intergenic, Non-coding, , ; rs9585057, 13:99431405,
AsA, non coding transcript exon variant, Non-coding, eQTL, HACER/GeneHancer; rs960709,
5:151081488, AsA, intron, Non-coding, eQTL, ; rs961253, 20:6423634, AsA, intergenic, Non-coding,
, ; rs9614123, 22:29975361, AsA, intron, Non-coding, eQTL, ; rs9630200, 11:120705136, AsA,
intron, Non-coding, eQTL, ; rs9644595, 8:16554275, AsA, intron, Non-coding, , ; rs9651118*,
1:11802157, AsA, intron, Non-coding, eQTL, ; rs971779, 12:65788675, AsA, intron, Non-coding,
eQTL, ; rs978639, 8:94214465, AsA, intron, Non-coding, eQTL, ; rs9815073, 3:188397894, AsA,
intron, Non-coding, , ; rs9832001, 3:178643995, AsA, intron, Non-coding, , ; rs9851386,
3:175047120, AsA, intron, Non-coding, , ; rs9855065, 3:119411294, AsA, intron, Non-coding, eQTL,
; rs9901869, 17:47497840, AsA, intergenic, Non-coding, eQTL, ; rs9905728, 17:17661209, AsA,
intergenic, Non-coding, eQTL, ; rs990887, 12:59152231, AsA, intergenic, Non-coding, , ; rs9923967,
16:75254286, AsA, intron, Non-coding, eQTL, ; rs999605, 7:51022368, AsA, intron, Non-coding,
eQTL, ; rs10052539, 5:92459013, AsA, lncRNA, Non-coding RNA, , ; rs10203477, 2:60877850,
AsA, lncRNA, Non-coding RNA, eQTL, ; rs10460003, 18:12747013, AsA, lncRNA, Non-coding
RNA, eQTL, ; rs10801129, 1:192552461, AsA, lncRNA, Non-coding RNA, eQTL,
HACER/GeneHancer; rs10878749, 12:68113359, AsA, lncRNA, Non-coding RNA, eQTL, ;
rs10991725, 9:90958576, AsA, lncRNA, Non-coding RNA, eQTL, HACER/GeneHancer;
rs10995309, 10:62801118, AsA, lncRNA, Non-coding RNA, eQTL, ; rs11030875, 11:29924160, AsA,
lncRNA, Non-coding RNA, , ; rs1111693, 13:40453366, AsA, lncRNA, Non-coding RNA, , ;
rs112021726, 5:40502876, AsA, lncRNA, Non-coding RNA, eQTL, ; rs11586962, 1:244024853, AsA,
lncRNA, Non-coding RNA, , ; rs11753807, 6:90321486, AsA, lncRNA, Non-coding RNA, , ;
rs11764334, 7:106606698, AsA, lncRNA, Non-coding RNA, , ; rs11773945, 8:128461433, AsA,
lncRNA, Non-coding RNA, , ; rs11789884, 9:14448319, AsA, lncRNA, Non-coding RNA, , ;
rs11847111, 14:30216302, AsA, lncRNA, Non-coding RNA, eQTL, ; rs11952467, 5:39912522, AsA,
lncRNA, Non-coding RNA, eQTL, ; rs12096737*, 1:226088853, AsA, lncRNA, Non-coding RNA,
eQTL, ; rs12261850, 10:6914224, AsA, lncRNA, Non-coding RNA, , ; rs12269979, 11:87960152,
AsA, lncRNA, Non-coding RNA, , ; rs12573430, 10:79041189, AsA, lncRNA, Non-coding RNA,
eQTL, ; rs12750006, 1:38066073, AsA, lncRNA, Non-coding RNA, , ; rs12963692, 18:73923961,
AsA, lncRNA, Non-coding RNA, , ; rs1459042, 6:21236316, AsA, lncRNA, Non-coding RNA, , ;
rs1484481, 2:66009628, AsA, lncRNA, Non-coding RNA, , ; rs1585576, 4:178004870, AsA, lncRNA,
Non-coding RNA, eQTL, ; rs1666795, 5:8539701, AsA, lncRNA, Non-coding RNA, eQTL, ;
rs16822996, 3:153671981, AsA, lncRNA, Non-coding RNA, eQTL, ; rs16830407, 2:198760429, AsA,
lncRNA, Non-coding RNA, , ; rs16905838, 8:136412224, AsA, lncRNA, Non-coding RNA, , ;
rs17005992, 4:122662460, AsA, lncRNA, Non-coding RNA, eQTL, ; rs17124028, 14:88078808, AsA,
lncRNA, Non-coding RNA, eQTL, ; rs17249487, 8:74149115, AsA, lncRNA, Non-coding RNA,
eQTL, ; rs17585578, 8:16877938, AsA, lncRNA, Non-coding RNA, , ; rs17645346, 5:135904064,
AsA, lncRNA, Non-coding RNA, eQTL, ; rs17744605, 18:32538805, AsA, lncRNA, Non-coding
RNA, , ; rs1857437, 9:19881866, AsA, lncRNA, Non-coding RNA, , ; rs1887250, 1:82488677, AsA,
lncRNA, Non-coding RNA, , ; rs1978421, 5:160456210, AsA, lncRNA, Non-coding RNA, eQTL,
HACER/GeneHancer; rs2051275, 21:25357150, AsA, lncRNA, Non-coding RNA, eQTL, ; rs224121,
10:62687592, AsA, lncRNA, Non-coding RNA, , HACER/GeneHancer; rs2280381, 16:85985027,
AsA, lncRNA, Non-coding RNA, eQTL, HACER/GeneHancer; rs2340713, 16:5021669, AsA,
lncRNA, Non-coding RNA, eQTL, ; rs2371516, 12:97051539, AsA, lncRNA, Non-coding RNA, , ;
rs241001, 6:111258795, AsA, lncRNA, Non-coding RNA, eQTL, ; rs2431697*, 5:160452971, AsA,
lncRNA, Non-coding RNA, eQTL, HACER/GeneHancer; rs2556560, 15:44529645, AsA, lncRNA,
Non-coding RNA, eQTL, ; rs2697325, 2:145198076, AsA, lncRNA, Non-coding RNA, eQTL, ;
rs285476, 1:165513934, AsA, lncRNA, Non-coding RNA, eQTL, ; rs305094, 16:85944499, AsA,
lncRNA, Non-coding RNA, , ; rs372813, 10:130442933, AsA, lncRNA, Non-coding RNA, , ;
rs414031, 5:174933214, AsA, lncRNA, Non-coding RNA, , ; rs4275832, 15:97390145, AsA, lncRNA,
Non-coding RNA, , ; rs4419828, 8:125513821, AsA, lncRNA, Non-coding RNA, , ; rs4422297,
3:64719184, AsA, lncRNA, Non-coding RNA, eQTL, ; rs4979521, 9:115360349, AsA, lncRNA, Non-
coding RNA, eQTL, ; rs4983191, 14:26748337, AsA, lncRNA, Non-coding RNA, , ; rs5022837,
4:132617351, AsA, lncRNA, Non-coding RNA, , ; rs522833, 3:187959592, AsA, lncRNA, Non-
coding RNA, , ; rs6134366, 20:11812314, AsA, lncRNA, Non-coding RNA, eQTL, ; rs620438,
13:63253067, AsA, lncRNA, Non-coding RNA, eQTL, ; rs6808518, 3:160012951, AsA, lncRNA,
Non-coding RNA, eQTL, ; rs6869688*, 5:159456019, AsA, lncRNA, Non-coding RNA, , ; rs692890,
3:159981785, AsA, lncRNA, Non-coding RNA, eQTL, ; rs7102093, 11:115584367, AsA, lncRNA,
Non-coding RNA, , ; rs7135334, 12:70039414, AsA, lncRNA, Non-coding RNA, eQTL, ; rs7175787,
15:100858595, AsA, lncRNA, Non-coding RNA, , HACER/GeneHancer; rs72976788, 6:126845098,
AsA, lncRNA, Non-coding RNA, eQTL, ; rs749305, 15:24810131, AsA, lncRNA, Non-coding RNA,
eQTL, ; rs7675104, 4:130419213, AsA, lncRNA, Non-coding RNA, , ; rs7969592, 12:68185869,
AsA, lncRNA, Non-coding RNA, , ; rs7988075, 13:42371436, AsA, lncRNA, Non-coding RNA,
eQTL, ; rs806321, 13:50267187, AsA, lncRNA, Non-coding RNA, eQTL, HACER/GeneHancer;
rs849330, 7:28220693, AsA, lncRNA, Non-coding RNA, eQTL, ; rs871982, 8:16741131, AsA,
lncRNA, Non-coding RNA, , ; rs919037, 8:63934853, AsA, lncRNA, Non-coding RNA, , ; rs9310860,
3:28022297, AsA, lncRNA, Non-coding RNA, , ; rs9494892, 6:137902352, AsA, lncRNA, Non-
coding RNA, eQTL, ; rs9533618, 13:43798977, AsA, lncRNA, Non-coding RNA, eQTL,
HACER/GeneHancer; rs9533737, 13:43994752, AsA, lncRNA, Non-coding RNA, eQTL, ; rs954431,
15:38674564, AsA, lncRNA, Non-coding RNA, , ; rs9603697, 13:40209186, AsA, lncRNA, Non-
coding RNA, , ; rs1005283, 20:45348974, AsA, promoter, Regulatory, eQTL, ; rs1029561,
7:26593409, AsA, PFR, Regulatory, eQTL, ; rs10762485, 10:71859671, AsA, PFR, Regulatory,
eQTL, HACER/GeneHancer; rs11033004, 11:35106031, AsA, PFR, Regulatory, , ; rs11069363,
13:99434025, AsA, promoter, Regulatory, eQTL, ; rs11071657, 15:62141763, AsA, PFR, Regulatory,
eQTL, ; rs11085824, 19:12890733, AsA, TFBS, Regulatory, eQTL, ; rs1160119, 2:134018384, AsA,
enhancer, Regulatory, , ; rs11668429, 19:10505627, AsA, PFR, Regulatory, eQTL, ; rs11785067,
8:10564457, AsA, PFR, Regulatory, eQTL, ; rs12087257, 1:197909571, AsA, CTCF binding site,
Regulatory, eQTL, ; rs12574073, 11:128449583, AsA, PFR, Regulatory, , ; rs13012920, 2:180943401,
AsA, enhancer, Regulatory, , ; rs13252899*, 8:144350943, AsA, PFR, Regulatory, eQTL, ; rs137685,
22:39343623, AsA, TFBS, Regulatory, eQTL, HACER/GeneHancer; rs1471400, 4:87853095, AsA,
CTCP binding site, Regulatory, eQTL, ; rs16967039, 15:38588219, AsA, PFR, Regulatory, , ;
rs1796013, 12:116441671, AsA, PFR, Regulatory, , ; rs1864174, 5:83034532, AsA, PFR, Regulatory,
eQTL, ; rs1932648, 9:84255158, AsA, PFR, Regulatory, eQTL, ; rs1957108, 14:35377057, AsA, PFR,
Regulatory, eQTL, ; rs2065796, 13:20219730, AsA, PFR, Regulatory, , ; rs2128406, 3:65114322,
AsA, OCR, Regulatory, , ; rs2178835, 21:39112030, AsA, PFR, Regulatory, , ; rs223889,
16:57358329, AsA, promoter, Regulatory, eQTL, ; rs2357570, 8:66005760, AsA, PFR, Regulatory,
eQTL, ; rs2640607, 12:57637249, AsA, CTCF binding site, Regulatory, eQTL, ; rs2736335,
8:11483978, AsA, TFBS, Regulatory, eQTL, HACER/GeneHancer; rs2785202, 11:35063288, AsA,
PFR, Regulatory, eQTL, HACER/GeneHancer; rs2882969, 2:203781465, AsA, PFR, Regulatory,
eQTL, ; rs353589, 11:35099679, AsA, PFR, Regulatory, eQTL, HACER/GeneHancer; rs35373757,
4:26028739, AsA, TFBS, Regulatory, eQTL, ; rs3754094, 1:198638198, AsA, promoter, Regulatory, ,
; rs4285698, 1:206740107, AsA, PFR, Regulatory, , ; rs4385425, 7:50267738, AsA, promoter,
Regulatory, eQTL, ; rs4680899, 3:28078149, AsA, PFR, Regulatory, , ; rs4921293, 5:160501869,
AsA, PFR, Regulatory, , ; rs56377341, 11:2187438, AsA, TFBS, Regulatory, , ; rs5750398,
22:37219171, AsA, promoter, Regulatory, eQTL, ; rs59998610, 2:68419639, AsA, PFR, Regulatory,
eQTL, HACER/GeneHancer; rs6429366, 1:242603703, AsA, enhancer, Regulatory, , ; rs6509546,
19:51515220, AsA, CTCF binding site, Regulatory, eQTL, ; rs6548238, 2:634905, AsA, TFBS,
Regulatory, eQTL, HACER/GeneHancer; rs67482294, 19:18294806, AsA, promoter, Regulatory,
eQTL, ; rs6856121, 4:12118236, AsA, PFR, Regulatory, , ; rs6966824, 7:15158964, AsA, enhancer,
Regulatory, , ; rs73191659, 7:107835346, AsA, PFR, Regulatory, eQTL, ; rs73366469*, 7:74619286,
AsA, enhancer, Regulatory, eQTL, ; rs74060136, 14:68782412, AsA, promoter, Regulatory, , ;
rs743636, X:152914199, AsA, CTCF binding site, Regulatory, eQTL, ; rs844649, 1:173255204, AsA,
PFR, Regulatory, , ; rs854666, 17:36030108, AsA, PFR, Regulatory, eQTL, ; rs9315093,
13:31011791, AsA, PFR, Regulatory, , ; rs9323913, 14:94680973, AsA, TFBS, Regulatory, , ;
rs9328529, 9:131367703, AsA, PFR, Regulatory, , ; rs2476601, 1:113834946, EA, missense, Coding,
eQTL, ; rs12142199, 1:1313807, BA, 3′UTR, Coding, , ; rs928596, 1:155978084, EA, 5′UTR, Coding,
, ; rs1801274, 1:161509955, EA, missense, Coding, , ; rs1053093, 1:183935939, EA, 3′UTR, Coding, ,
; rs10916668, 1:19906593, EA, missense, Coding, eQTL, ; rs17617, 1:207480050, EA, missense,
Coding, , ; rs4844600, 1:207505962, EA, missense, Coding, , ; rs196432, 1:24535214, EA, missense,
Coding, eQTL, ; rs989767, 10:19817007, EA, 5′UTR, Coding, , ; rs2228054, 11:117993398, EA,
3′UTR, Coding, , ; rs7939069, 11:47666907, EA, Stop gained/nonsense, Coding, , ; rs968567,
11:61828092, EA, 5′UTR, Coding, eQTL, ; rs4944946, 11:71448383, EA, 5′UTR, Coding, , ;
rs11235715, 11:73296809, EA, synonymous, Coding, , ; rs11612569, 12:101407031, EA, 5′UTR,
Coding, , ; rs2230776, 12:121263878, EA, synonomous, Coding, eQTL, ; rs1059312, 12:128794319,
EA, synonomous, Coding, eQTL, ; rs1990313, 12:4626920, EA, missense, Coding, , ; rs7787,
13:20723059, EA, 3′UTR, Coding, eQTL, ; rs11647067, 16:23079657, EA, 3′UTR, Coding, eQTL, ;
rs7500321, 16:28965699, EA, 3′UTR, Coding, eQTL, ; rs1143679, 16:31265490, EA, missense,
Coding, , ; rs238224, 17:4960115, EA, 3′UTR, Coding, eQTL, ; rs3785437, 17:75910141, EA, 3′UTR,
Coding, eQTL, ; rs9303891, 18:213151, EA, 3′UTR, Coding, , ; rs7241191, 18:69856217, EA, 3′UTR,
Coding, eQTL, ; rs34536443, 19:10352442, EA, missense, Coding, , ; rs7249065, 19:1112944, EA,
synonomous, Coding, eQTL, ; rs34006827, 19:35858834, EA, synonymous, Coding, , ; rs230261,
19:35872568, EA, synonomous, Coding, eQTL, ; rs7252175, 19:45629998, EA, missense, Coding, , ;
rs281392, 19:48661695, EA, synonomous, Coding, eQTL, ; rs724710, 2:111150114, EA, 3′UTR,
Coding, , ; rs35667974, 2:162268127, EA, missense, Coding, , ; rs7566538, 2:240715541, EA, 3′UTR,
Coding, , ; rs10933559, 2:241468331, EA, synonomous, Coding, eQTL, ; rs2287339, 2:53765456, EA,
synonymous, Coding, , ; rs6032198, 20:45447208, EA, 3′UTR, Coding, , ; rs7679, 20:45947863, EA,
3′UTR, Coding, eQTL, ; rs464694, 22:21447200, EA, 3′UTR, Coding, eQTL, ; rs1132200,
3:119431989, EA, missense, Coding, eQTL, ; rs1131265, 3:119503609, EA, synonymous, Coding, , ;
rs35370245, 3:197044683, EA, synonymous, Coding, , ; rs11539148, 3:49101377, EA, missense,
Coding, , ; rs10516487, 4:101829919, EA, missense, Coding, , ; rs3733345, 4:960459, EA, 3′UTR,
Coding, eQTL, ; rs79074797, 6:111268561, EA, 3′UTR, Coding, eQTL, ; rs2230926, 6:137874929,
EA, missense, Coding, , ; rs11755393, 6:34856859, EA, missense, Coding, eQTL, ; rs13205210,
6:34864079, EA, missense, Coding, eQTL, ; rs1194, 6:35295778, EA, 3′UTR, Coding, eQTL, ;
rs2229524, 6:85489525, EA, missense, Coding, eQTL, ; rs11171, 7:100888489, EA, synonymous,
Coding, , ; rs10271373, 7:139045049, EA, 3′UTR, Coding, eQTL, ; rs2953898, 8:56068244, EA,
3′UTR, Coding, eQTL, ; rs1051624, 8:94130944, EA, missense, Coding, , ; rs2241003, 9:120904499,
EA, 3′UTR, Coding, eQTL, ; rs3739817, 9:127824409, EA, synonymous, Coding, , ; rs11145974,
9:136422292, EA, 3′UTR, Coding, eQTL, ; rs1052690, 9:83643770, EA, missense, Coding, , ;
rs2095731, 1:111491854, EA, Intron, Non-coding, , ; rs4644492, 1:112478596, EA, intron, Non-
coding, eQTL, ; rs1237290, 1:113592525, EA, intron, Non-coding, eQTL, ; rs6679677, 1:113761186,
EA, intergenic, Non-coding, eQTL, ; rs9887785, 1:116714075, EA, Intron, Non-coding, , ; rs835574,
1:119920607, EA, Intron, Non-coding, , ; rs4579751, 1:14027816, EA, Intron, Non-coding, , ;
rs17362529, 1:147765830, EA, Intron, Non-coding, , ; rs6663448, 1:152922151, EA, intergenic, Non-
coding, eQTL, ; rs11804305, 1:154452021, EA, intron, Non-coding, eQTL, ; rs4971066, 1:155133406,
EA, Intron, Non-coding, , ; rs9427315, 1:157801238, EA, Intron, Non-coding, , ; rs3766374,
1:160750764, EA, Intron, Non-coding, , ; rs116660017, 1:161499981, EA, intergenic, Non-coding,
eQTL, HACER; rs10800309, 1:161502368, EA, intergenic, Non-coding, eQTL, ; rs7540556,
1:169562204, EA, intron, Non-coding, eQTL, ; rs10798176, 1:172706385, EA, Intergenic, Non-
coding, , HACER; rs2205960, 1:173222336, EA, Intergenic, Non-coding, , ; rs10753074,
1:173377204, EA, Intergenic, Non-coding, , ; rs10922144, 1:196854170, EA, intron, Non-coding,
eQTL, ; rs12408855, 1:19958381, EA, Intergenic, Non-coding, , ; rs10800816, 1:202156235, EA,
intron, Non-coding, eQTL, ; rs12145347, 1:204275345, EA, Intron, Non-coding, , ; rs3122605,
1:206781696, EA, Intergenic, Non-coding, , ; rs17546779, 1:215058065, EA, Intron, Non-coding, , ;
rs6700189, 1:226072106, EA, Intron, Non-coding, , ; rs6673129, 1:2271335, EA, Intron, Non-coding,
, ; rs16858884, 1:233223838, EA, Intron, Non-coding, , ; rs2891663, 1:235870974, EA, Intron, Non-
coding, , ; rs6673121, 1:235877648, EA, Intron, Non-coding, , ; rs9428886, 1:241691507, EA, Intron,
Non-coding, , ; rs12743484, 1:26009438, EA, intergenic, Non-coding, eQTL, HACER; rs114800103,
1:2861494, EA, Intergenic, Non-coding, , ; rs1372372, 1:29648100, EA, Intergenic, Non-coding, , ;
rs10889449, 1:38185936, EA, Intergenic, Non-coding, , ; rs2982846, 1:51813978, EA, intron, Non-
coding, eQTL, ; rs34743561, 1:51869424, EA, Intron, Non-coding, , ; rs4927193, 1:55044199, EA,
Intron, Non-coding, , ; rs10889681, 1:67333487, EA, intron, Non-coding, eQTL, ; rs3790564,
1:67340771, EA, intron, Non-coding, eQTL, ; rs10889687, 1:67444099, EA, intergenic, Non-coding,
eQTL, ; rs7522008, 1:77119161, EA, intron, Non-coding, eQTL, ; rs2044264, 1:88500115, EA,
Intron, Non-coding, , ; rs11803383, 1:92445799, EA, Intergenic, Non-coding, , ; rs6662618,
1:92469854, EA, intergenic, Non-coding, eQTL, ; rs12738833, 1:92653561, EA, intron, Non-coding,
eQTL, ; rs11164848, 1:92955553, EA, Intron, Non-coding, , ; rs17100664, 1:98118847, EA,
Intergenic, Non-coding, , ; rs116818286, 1:98130791, EA, Intergenic, Non-coding, , ; rs1350176,
1:99029579, EA, Intron, Non-coding, , ; rs12248123, 10:102975609, EA, intron, Non-coding, eQTL, ;
rs9783215, 10:11223393, EA, Intron, Non-coding, , ; rs11598094, 10:122347530, EA, intergenic,
Non-coding, eQTL, ; rs7916519, 10:23177805, EA, Intergenic, Non-coding, , ; rs2928403,
10:48810374, EA, intron, Non-coding, eQTL, ; rs2663054, 10:48893932, EA, Intron, Non-coding, , ;
rs1913517, 10:48911009, EA, Intron, Non-coding, , ; rs1670807, 10:54922465, EA, Intron, Non-
coding, , ; rs2891551, 10:54927865, EA, Intron, Non-coding, , ; rs17805636, 10:60206610, EA,
Intron, Non-coding, , ; rs2787733, 10:61488649, EA, Intergenic, Non-coding, , ; rs2256597,
10:61503536, EA, intergenic, Non-coding, eQTL, ; rs9415635, 10:61993723, EA, Intron, Non-coding,
, ; rs12764378, 10:62040245, EA, Intron, Non-coding, , ; rs116967545, 10:62744071, EA, Intron,
Non-coding, , ; rs17813747, 10:63406910, EA, Intron, Non-coding, , ; rs112123005, 10:6430530, EA,
Intron, Non-coding, , ; rs10761901, 10:65045258, EA, Intergenic, Non-coding, , ; rs113304138,
10:6522315, EA, Intron, Non-coding, , ; rs7915387, 10:71250267, EA, intron, Non-coding, eQTL, ;
rs4363498, 10:7783567, EA, intron, Non-coding, eQTL, ; rs7900818, 10:82334296, EA, Intron, Non-
coding, , ; rs3862550, 10:82335015, EA, Intron, Non-coding, , ; rs6586128, 10:88374504, EA, Intron,
Non-coding, , ; rs115333320, 11:103505267, EA, Intergenic, Non-coding, , ; rs501192, 11:105029658,
EA, Intron, Non-coding, , ; rs526151, 11:116366058, EA, Intergenic, Non-coding, , ; rs6589566,
11:116781707, EA, intron, Non-coding, eQTL, ; rs688161, 11:118807001, EA, Intergenic, Non-
coding, , HACER; rs12575600, 11:128454974, EA, Intron, Non-coding, , ; rs633040, 11:131861532,
EA, Intron, Non-coding, , ; rs7114131, 11:14708787, EA, Intron, Non-coding, , ; rs11024600,
11:18274263, EA, intergenic, Non-coding, eQTL, ; rs7104239, 11:2217575, EA, Intergenic, Non-
coding, , ; rs12290504, 11:25988309, EA, Intergenic, Non-coding, , ; rs500600, 11:30459510, EA,
intron, Non-coding, eQTL, ; rs507230, 11:35107625, EA, Intergenic, Non-coding, , ; rs7114911,
11:42568107, EA, Intergenic, Non-coding, , ; rs4547082, 11:42602645, EA, Intergenic, Non-coding, ,
; rs4963128, 11:589564, EA, intron, Non-coding, eQTL, ; rs2396545, 11:601785, EA, intron, Non-
coding, eQTL, ; rs511580, 11:61068124, EA, Intergenic, Non-coding, , ; rs11246217, 11:623765, EA,
Intron, Non-coding, , ; rs645078, 11:64367826, EA, intron, Non-coding, eQTL, ; rs4930194,
11:64542619, EA, Intergenic, Non-coding, , ; rs12801636, 11:65623846, EA, intergenic, Non-coding,
eQTL, ; rs12417865, 11:69546618, EA, Intergenic, Non-coding, , ; rs497356, 11:69552407, EA,
Intergenic, Non-coding, , ; rs12422045, 11:71443774, EA, intron, Non-coding, eQTL, ; rs4459332,
11:72797222, EA, Intron, Non-coding, , ; rs7946044, 11:78579392, EA, Intron, Non-coding, , ;
rs61883873, 11:78580147, EA, Intron, Non-coding, , ; rs2156832, 11:81551992, EA, Intron, Non-
coding, , ; rs1894147, 11:81566879, EA, Intergenic, Non-coding, , ; rs12281485, 11:95603652, EA,
Intron, Non-coding, , ; rs10735418, 12:106949598, EA, intergenic, Non-coding, eQTL, ; rs17630235,
12:112153882, EA, intergenic, Non-coding, eQTL, ; rs7970893, 12:112952874, EA, intron, Non-
coding, eQTL, ; rs16946868, 12:116629982, EA, Intergenic, Non-coding, , ; rs113420846,
12:116632853, EA, Intergenic, Non-coding, , ; rs2541117, 12:11678274, EA, Intron, Non-coding, , ;
rs904661, 12:118144055, EA, intron, Non-coding, eQTL, ; rs11065513, 12:121262302, EA, Intron,
Non-coding, , ; rs116912920, 12:123215264, EA, Intron, Non-coding, , ; rs11059927, 12:128809788,
EA, intron, Non-coding, eQTL, ; rs1385374, 12:128816149, EA, intron, Non-coding, eQTL, ;
rs10773782, 12:130425392, EA, Intron, Non-coding, , ; rs2220168, 12:26505546, EA, Intron, Non-
coding, , ; rs11050953, 12:30559037, EA, Intergenic, Non-coding, , ; rs12322502, 12:30568907, EA,
Intergenic, Non-coding, , ; rs7311756, 12:39976371, EA, Intron, Non-coding, , ; rs3925535,
12:40088092, EA, intron, Non-coding, eQTL, ; rs11181779, 12:42888706, EA, Intergenic, Non-
coding, , ; rs17105765, 12:53992208, EA, Intron, Non-coding, , ; rs116844988, 12:57554511, EA,
Intron, Non-coding, , ; rs1393988, 12:60593582, EA, Intergenic, Non-coding, , ; rs2654676,
12:60607200, EA, Intergenic, Non-coding, , ; rs6581431, 12:61775905, EA, Intron, Non-coding, , ;
rs7486855, 12:68044153, EA, Intron, Non-coding, , ; rs11177019, 12:68048711, EA, Intron, Non-
coding, , ; rs12298456, 12:96897861, EA, intergenic, Non-coding, eQTL, ; rs11616341,
13:101068677, EA, Intron, Non-coding, , ; rs12865863, 13:101069350, EA, Intron, Non-coding, , ;
rs9520836, 13:108304715, EA, Intron, Non-coding, , ; rs7982251, 13:28335698, EA, Intron, Non-
coding, , ; rs7982957, 13:28335733, EA, Intron, Non-coding, , ; rs238353, 13:42270188, EA, Intron,
Non-coding, , ; rs9568353, 13:49907577, EA, Intergenic, Non-coding, , ; rs4942903, 13:50132066,
EA, Intron, Non-coding, , ; rs4885144, 13:74010603, EA, Intergenic, Non-coding, , ; rs696780,
13:77067114, EA, Intron, Non-coding, , ; rs1751036, 13:95066411, EA, Intron, Non-coding, , ;
rs1189433, 13:95086069, EA, Intron, Non-coding, , ; rs285049, 13:98108888, EA, Intergenic, Non-
coding, , ; rs285050, 13:98110199, EA, Intergenic, Non-coding, , ; rs7325747, 13:99377678, EA,
Intron, Non-coding, , ; rs12148050, 14:102797451, EA, Intron, Non-coding, , ; rs2015407,
14:102808210, EA, Intron, Non-coding, , ; rs12588573, 14:20678425, EA, Intergenic, Non-coding, ,
HACER; rs10483515, 14:39986367, EA, Intergenic, Non-coding, , ; rs6574349, 14:77031323, EA,
Intron, Non-coding, , HACER; rs12050151, 14:80902473, EA, intron, Non-coding, eQTL, ;
rs1952537, 14:83787402, EA, Intergenic, Non-coding, , ; rs1958442, 14:83797557, EA, Intergenic,
Non-coding, , ; rs454425, 14:87938371, EA, intron, Non-coding, eQTL, ; rs7150563, 14:98296008,
EA, Intergenic, Non-coding, , ; rs7179733, 15:32081490, EA, Intron, Non-coding, , ; rs6492983,
15:41143851, EA, intergenic, Non-coding, eQTL, HACER; rs965355, 15:59767625, EA, Intron, Non-
coding, , HACER; rs1869486, 15:60502608, EA, Intron, Non-coding, , ; rs12916690, 15:60692071,
EA, Intron, Non-coding, , ; rs35281701, 15:69699715, EA, Intron, Non-coding, , ; rs16953685
15:69741749, EA, Intron, Non-coding, , ; rs1378942, 15:74785026, EA, intron, Non-coding, eQTL,
HACER; rs117626207, 15:78905081, EA, Intron, Non-coding, , ; rs2732159, 15:83585510, EA,
intron, Non-coding, eQTL, ; rs11074015, 15:91727107, EA, Intergenic, Non-coding, , ; rs9652601,
16:11080508, EA, intron, Non-coding, eQTL, ; rs12599402, 16:11096031, EA, intron, Non-coding,
eQTL, ; rs78318981, 16:11249165, EA, Intron, Non-coding, , ; rs4513082, 16:12784735, EA, Intron,
Non-coding, , ; rs4369667, 16:25859468, EA, Intron, Non-coding, , ; rs62057174, 16:30655482, EA,
Intron, Non-coding, , ; rs8048448, 16:30680887, EA, Intergenic, Non-coding, , ; rs6565201,
16:30735870, EA, Intron, Non-coding, , ; rs7186889, 16:50058380, EA, intron, Non-coding, eQTL, ;
rs223881, 16:57352654, EA, intergenic, Non-coding, eQTL, ; rs223883, 16:57354818, EA, Intron,
Non-coding, , ; rs12444634, 16:5737586, EA, Intron, Non-coding, , ; rs1974876, 16:58251299, EA,
Intron, Non-coding, , ; rs11076230, 16:58255081, EA, Intron, Non-coding, , ; rs17335437,
16:62292810, EA, Intergenic, Non-coding, , ; rs12918180, 16:6514547, EA, Intron, Non-coding, , ;
rs8061145, 16:65747835, EA, Intergenic, Non-coding, , ; rs4783754, 16:67324913, EA, Intron, Non-
coding, , ; rs2271293, 16:67868167, EA, intron, Non-coding, eQTL, ; rs1170436, 16:68573583, EA,
intron, Non-coding, eQTL, ; rs11640251, 16:70708633, EA, Intron, Non-coding, , ; rs12597935,
16:73113585, EA, Intron, Non-coding, , ; rs4888360, 16:75220686, EA, intron, Non-coding, eQTL, ;
rs2136349, 16:79327592, EA, Intergenic, Non-coding, , ; rs11117431, 16:85981710, EA, Intron, Non-
coding, , HACER; rs72807059, 16:85988491, EA, Intron, Non-coding, , ; rs1024580, 17:12951199,
EA, Intron, Non-coding, , ; rs55873962, 17:22176898, EA, Intron, Non-coding, , ; rs113815962,
17:27738430, EA, Intergenic, Non-coding, , ; rs56114296, 17:27783722, EA, Intron, Non-coding, , ;
rs8066255, 17:38254553, EA, intron, Non-coding, eQTL, ; rs72832915, 17:39678555, EA, Intron,
Non-coding, , ; rs4252665, 17:39729130, EA, intergenic, Non-coding, eQTL, ; rs8079075,
17:39854562, EA, Intron, Non-coding, , ; rs726848, 17:40719432, EA, intron, Non-coding, eQTL,
HACER; rs12150495, 17:42236412, EA, intron, Non-coding, eQTL, ; rs56228409, 17:42767578, EA,
intergenic, Non-coding, eQTL, ; rs74597067, 17:43315891, EA, Intergenic, Non-coding, , HACER;
rs4474733, 17:43356292, EA, intergenic, Non-coding, eQTL, ; rs183211, 17:46710944, EA, intron,
Non-coding, eQTL, ; rs7214635, 17:4697725, EA, intron, Non-coding, eQTL, ; rs203102,
17:51943839, EA, Intron, Non-coding, , ; rs1292043, 17:59856808, EA, intergenic, Non-coding,
eQTL, ; rs930297, 17:75408456, EA, intergenic, Non-coding, eQTL, ; rs894956, 17:76930620, EA,
Intron, Non-coding, , ; rs17681910, 18:13406060, EA, Intron, Non-coding, , ; rs7240797,
18:30213665, EA, Intergenic, Non-coding, , ; rs8085075, 18:35760285, EA, Intergenic, Non-coding, ,
; rs2959219, 18:38418662, EA, Intergenic, Non-coding, , ; rs9952980, 18:45308832, EA, Intron, Non-
coding, , ; rs1472788, 18:5209021, EA, Intron, Non-coding, , ; rs73440898, 18:58113886, EA, Intron,
Non-coding, , ; rs631574, 18:7812194, EA, Intron, Non-coding, , ; rs364418, 18:7813241, EA, Intron,
Non-coding, , ; rs74908652, 19:10312296, EA, intron, Non-coding, eQTL, ; rs34725611,
19:10366391, EA, Intron, Non-coding, , ; rs147622113, 19:10661265, EA, Intron, Non-coding, , ;
rs2304087, 19:10996457, EA, intron, Non-coding, eQTL, ; rs11673460, 19:18080811, EA, Intron,
Non-coding, , ; rs425648, 19:18091302, EA, intron, Non-coding, eQTL, HACER; rs12971295,
19:18406521, EA, Intergenic, Non-coding, , ; rs13344313, 19:18406957, EA, intergenic, Non-coding,
eQTL, ; rs12986413, 19:2170955, EA, intron, Non-coding, eQTL, ; rs12978179, 19:2172379, EA,
Intron, Non-coding, , ; rs10411749, 19:29615062, EA, Intron, Non-coding, , ; rs11671087,
19:39271150, EA, intergenic, Non-coding, eQTL, ; rs7359953, 19:39273073, EA, Intron, Non-coding,
, ; rs2082418, 19:39274426, EA, Intron, Non-coding, , ; rs16979595, 19:44974124, EA, intron, Non-
coding, eQTL, ; rs7248181, 19:47084945, EA, Intron, Non-coding, , ; rs2303108, 19:47086638, EA,
Intron, Non-coding, , ; rs113867041, 19:49384562, EA, Intron, Non-coding, , ; rs58089667,
19:54424853, EA, Intron, Non-coding, , ; rs73066671, 19:55185316, EA, intron, Non-coding, eQTL, ;
rs56154925, 19:55226430, EA, intergenic, Non-coding, eQTL, ; rs2061776, 19:8137489, EA, Intron,
Non-coding, , ; rs2238573, 19:945013, EA, Intron, Non-coding, , ; rs73966422, 2:100137627, EA,
Intron, Non-coding, , ; rs12712067, 2:100147438, EA, Intron, Non-coding, , ; rs1369481,
2:100895497, EA, Intron, Non-coding, , ; rs116101946, 2:101778406, EA, Intron, Non-coding, , ;
rs12613330, 2:102047999, EA, Intergenic, Non-coding, , ; rs11681718, 2:102434684, EA, intron,
Non-coding, eQTL, ; rs3769503, 2:105860829, EA, intron, Non-coding, eQTL, ; rs2723196,
2:112932500, EA, intergenic, Non-coding, eQTL, ; rs3109154, 2:132584956, EA, Intron, Non-coding,
, ; rs2460382, 2:134256545, EA, Intron, Non-coding, , ; rs10496726, 2:134287679, EA, Intron, Non-
coding, , ; rs11887156, 2:134308905, EA, Intron, Non-coding, , ; rs12992815, 2:135805678, EA,
intron, Non-coding, eQTL, ; rs55634455, 2:135836124, EA, Intron, Non-coding, , ; rs12464087,
2:15762788, EA, Intergenic, Non-coding, , ; rs7580177, 2:1689263, EA, intron, Non-coding, eQTL, ;
rs746179, 2:173161596, EA, intron, Non-coding, eQTL, ; rs2600694, 2:174732104, EA, Intron, Non-
coding, , ; rs7606861, 2:18302778, EA, Intron, Non-coding, , ; rs10931167, 2:185276686, EA,
Intergenic, Non-coding, , ; rs11685321, 2:188532344, EA, intron, Non-coding, eQTL, ; rs4476350,
2:18919100, EA, Intergenic, Non-coding, , ; rs11096566, 2:18921106, EA, Intergenic, Non-coding, , ;
rs932169, 2:191064552, EA, Intron, Non-coding, , ; rs7568275, 2:191101726, EA, Intron, Non-
coding, , ; rs6738825, 2:198032171, EA, intron, Non-coding, eQTL, ; rs3769433, 2:200468278, EA,
intron, Non-coding, eQTL, ; rs16837131, 2:201309089, EA, intron, Non-coding, eQTL, ; rs2019097,
2:201328740, EA, Intron, Non-coding, , ; rs4666298, 2:20220971, EA, Intron, Non-coding, , ;
rs6756819, 2:20371912, EA, Intergenic, Non-coding, , ; rs10197010, 2:203875448, EA, Intergenic,
Non-coding, , ; rs1012525, 2:206672949, EA, Intron, Non-coding, , ; rs10197379, 2:213196515, EA,
Intergentic, Non-coding, , ; rs13001747, 2:213215069, EA, Intergenic, Non-coding, , ; rs57545959,
2:218182995, EA, Intergenic, Non-coding, , ; rs13394788, 2:228235251, EA, Intergenic, Non-coding,
, ; rs1609265, 2:228276234, EA, Intergenic, Non-coding, , ; rs2675954, 2:232885917, EA, intron,
Non-coding, eQTL, ; rs12692144, 2:234539945, EA, Intergenic, Non-coding, , ; rs7566072,
2:235294498, EA, Intergenic, Non-coding, , ; rs12474299, 2:238765748, EA, Intergenic, Non-coding,
, ; rs114568253, 2:25237494, EA, Intron, Non-coding, , ; rs1355208, 2:30222456, EA, Intergenic,
Non-coding, , ; rs2161070, 2:41003158, EA, Intergenic, Non-coding, , ; rs6756736, 2:43331604, EA,
Intron, Non-coding, , ; rs1470475, 2:4776483, EA, Intergenic, Non-coding, , ; rs75096128,
2:53758705, EA, Intron, Non-coding, , ; rs10209543, 2:557205, EA, Intergenic, Non-coding, , ;
rs16864190, 2:5778284, EA, Intergenic, Non-coding, , ; rs62149377, 2:60759441, EA, intron, Non-
coding, eQTL, ; rs2600669, 2:61174161, EA, intergenic, Non-coding, eQTL, ; rs10197217,
2;62269532, EA, intergenic, Non-coding, eQTL, ; rs2576837, 2:64112143, EA, Intron, Non-coding, , ;
rs6743585, 2:65328586, EA, Intron, Non-coding, , ; rs2244364, 2:65483194, EA, intron, Non-coding,
eQTL, HACER; rs55869446, 2:68359287, EA, Intron, Non-coding, , HACER; rs1016350,
2:68374684, EA, intron, Non-coding, eQTL, ; rs13000817, 2:70046575, EA, intron, Non-coding,
eQTL, ; rs17759986, 2:84675559, EA, Intron, Non-coding, , ; rs6133922, 20:10420425, EA, Intron,
Non-coding, , ; rs750402, 20:32658764, EA, intron, Non-coding, eQTL, ; rs209665, 20:33541083, EA,
intron, Non-coding, eQTL, ; rs209677, 20:33552481, EA, Intron, Non-coding, , ; rs6088765,
20:35211477, EA, intron, Non-coding, eQTL, HACER; rs2425223, 20:36373117, EA, Intron, Non-
coding, , ; rs8116375, 20:43388433, EA, Intron, Non-coding, , ; rs6073958, 20:45923216, EA, Intron,
Non-coding, , ; rs4810485, 20:46119308, EA, intron, Non-coding, eQTL, ; rs562954, 20:49475539,
EA, intron, Non-coding, eQTL, ; rs6126200, 20:51263325, EA, Intergenic, Non-coding, , ; rs1546913,
20:57578979, EA, Intergenic, Non-coding, , ; rs7264711, 20:8650297, EA, Intron, Non-coding, , ;
rs2277802, 21:42409237, EA, intron, Non-coding, eQTL, ; rs9784212, 21:44279896, EA, Intergenic,
Non-coding, , ; rs17413405, 22:28454628, EA, Intron, Non-coding, , ; rs73161005, 22:29747251, EA,
intron, Non-coding, eQTL, ; rs11705183, 22:39352366, EA, intron, Non-coding, eQTL, ; rs2958647,
22:39895135, EA, Intron, Non-coding, , ; rs533852, 3:101275653, EA, intron, Non-coding, eQTL, ;
rs1707603, 3:101928244, EA, Intergenic, Non-coding, , HACER; rs9861188, 3:117731108, EA,
Intron, Non-coding, , ; rs1534154, 3:119592183, EA, intergenic, Non-coding, eQTL, ; rs4678000,
3:122171888, EA, Intergenic, Non-coding, , ; rs17036143, 3:12253228, EA, intergenic, Non-coding,
eQTL, ; rs78481160, 3:159980382, EA, Intron, Non-coding, , ; rs1983208, 3:162036321, EA,
Intergenic, Non-coding, , ; rs9884026, 3:162043990, EA, Intergenic, Non-coding, , ; rs7639882,
3:16943780, EA, Intron, Non-coding, , ; rs1997392, 3:169791864, EA, Intron, Non-coding, , ;
rs559311, 3:170733798, EA, Intron, Non-coding, , ; rs9880289, 3:171213910, EA, Intron, Non-
coding, , ; rs13095186, 3:187976979, EA, Intergenic, Non-coding, , ; rs4686912, 3:187984423, EA,
Intergenic, Non-coding, , ; rs9827931, 3:21257148, EA, Intergenic, Non-coding, , ; rs6780105,
3:25262214, EA, Intron, Non-coding, , ; rs376072, 3:28034792, EA, Intron, Non-coding, , ;
rs9821576, 3:29886828, EA, Intron, Non-coding, , ; rs9855205, 3:45894206, EA, Intron, Non-coding,
, ; rs9823523, 3:45895625, EA, Intron, Non-coding, , ; rs62262116, 3:49963719, EA, intron, Non-
coding, eQTL, ; rs79821347, 3:50494404, EA, Intron, Non-coding, , ; rs7625006, 3:51335509, EA,
intron, Non-coding, eQTL, ; rs357999, 3:55082476, EA, Intergenic, Non-coding, , ; rs4283545,
3:57166730, EA, Intron, Non-coding, , ; rs9311929, 3:65009882, EA, intron, Non-coding, eQTL, ;
rs10510388, 3:8215887, EA, Intron, Non-coding, , ; rs4426778, 4:101859567, EA, Intron, Non-
coding, , ; rs4355385, 4:10565335, EA, Intron, Non-coding, , ; rs61794854, 4:10620968, EA, Intron,
Non-coding, , ; rs56042874, 4:113795208, EA, Intergenic, Non-coding, , ; rs71606243, 4:121949298,
EA, Intron, Non-coding, , ; rs1121242, 4:122406061, EA, Intron, Non-coding, , ; rs11724582,
4:122470309, EA, Intergenic, Non-coding, , ; rs907715, 4:122613898, EA, Intron, Non-coding, , ;
rs4580644, 4:15783578, EA, intron, Non-coding, eQTL, ; rs1860598, 4:17655737, EA, Intron, Non-
coding, , ; rs13123614, 4:23636476, EA, intron, Non-coding, eQTL, ; rs4293757, 4:30941587, EA,
Intron, Non-coding, , ; rs4974949, 4:39812444, EA, Intergenic, Non-coding, , ; rs73245892,
4:46914781, EA, Intron, Non-coding, , ; rs7439087, 4:47394983, EA, Intron, Non-coding, , ;
rs2055802, 4:48208988, EA, Intron, Non-coding, , ; rs7692950, 4:48217324, EA, Intron, Non-coding,
, ; rs4234726, 4:6271161, EA, intron, Non-coding, eQTL, ; rs11722709, 4:64910111, EA, Intergenic,
Non-coding, , ; rs907526, 4:7083108, EA, intergenic, Non-coding, eQTL, ; rs1441904, 4:76230727,
EA, intron, Non-coding, eQTL, ; rs4859711, 4:76744126, EA, Intron, Non-coding, , ; rs1072626,
4:99486110, EA, intron, Non-coding, eQTL, ; rs7442295, 4:9964756, EA, Intron, Non-coding, , ;
rs6886392, 5:100800161, EA, Intergenic, Non-coding, , ; rs255920, 5:112048742, EA, Intergenic,
Non-coding, , ; rs255915, 5:112051805, EA, Intergenic, Non-coding, , ; rs7726414, 5:134096143, EA,
Intergenic, Non-coding, , HACER; rs1012172, 5:150334831, EA, Intron, Non-coding, , ; rs6579789,
5:150335701, EA, Intron, Non-coding, , ; rs7712962, 5:157709899, EA, intergenic, Non-coding,
eQTL, ; rs80179578, 5:160436302, EA, Intergenic, Non-coding, , ; rs2431098, 5:160460329, EA,
Intron, Non-coding, , HACER; rs11750159, 5:172488680, EA, Intergenic, Non-coding, , ; rs10085092
5:172489579, EA, Intergenic, Non-coding, , ; rs7714375, 5:3056900, EA, Intergenic, Non-coding, , ;
rs1496453, 5:55500967, EA, intron, Non-coding, eQTL, ; rs424561, 5:56118682, EA, Intron, Non-
coding, , ; rs415407, 5:56119129, EA, Intron, Non-coding, , ; rs11134150, 5:6223920, EA, Intergenic,
Non-coding, , ; rs2455331, 5:643478, EA, intron, Non-coding, eQTL, ; rs422258, 5:72537896, EA,
Intergenic, Non-coding, , ; rs33421, 5:72551450, EA, Intergenic, Non-coding, , ; rs266433,
5:72875888, EA, intron, Non-coding, eQTL, ; rs268794, 5:72940941, EA, Intergenic, Non-coding, ,
HACER; rs3864261, 5:73062427, EA, intron, Non-coding, eQTL, ; rs13172944, 5:8031286, EA,
Intergenic, Non-coding, , ; rs2624217, 5:87149815, EA, Intergenic, Non-coding, , ; rs12526490,
6:106115679, EA, Intron, Non-coding, , ; rs548234, 6:106120159, EA, Intron, Non-coding, , ;
rs9373839, 6:106207742, EA, Intron, Non-coding, , ; rs2299864, 6:106220119, EA, Intron, Non-
coding, , ; rs436872, 6:109231870, EA, Intron, Non-coding, , ; rs1360328, 6:126143090, EA, Intron,
Non-coding, , ; rs11154801, 6:135418217, EA, intron, Non-coding, eQTL, ; rs2327832, 6:137651931,
EA, Intergenic, Non-coding, , ; rs77000060, 6:137916852, EA, Intergenic, Non-coding, , ; rs7770235,
6:141201299, EA, Intergenic, Non-coding, , ; rs7764429, 6:14595206, EA, Intergenic, Non-coding, , ;
rs1267489, 6:14719479, EA, Intron, Non-coding, , ; rs17079296, 6:150090941, EA, intergenic, Non-
coding, eQTL, ; rs138584427, 6:151460644, EA, Intron, Non-coding, , ; rs6557142, 6:151462914, EA,
intron, Non-coding, eQTL, ; rs9476757, 6:15157058, EA, Intergenic, Non-coding, , ; rs12211203,
6:153695801, EA, Intergenic, Non-coding, , ; rs12207692, 6:153697279, EA, Intergenic, Non-coding,
, ; rs11756587, 6:158151943, EA, intron, Non-coding, eQTL, ; rs909788, 6:16636230, EA, Intron,
Non-coding, , ; rs78209349, 6:167021118, EA, Intron, Non-coding, , ; rs6912394, 6:16945766, EA,
Intergenic, Non-coding, , ; rs910425, 6:170343103, EA, Intron, Non-coding, , ; rs6913643,
6:170503067, EA, Intergenic, Non-coding, , ; rs9366394, 6:21343804, EA, Intergenic, Non-coding, , ;
rs150173969, 6:21345451, EA, Intergenic, Non-coding, , ; rs926571, 6:22673224, EA, Intergenic,
Non-coding, , ; rs10498722, 6:25186284, EA, intron, Non-coding, eQTL, ; rs4712969, 6:25763964,
EA, intron, Non-coding, eQTL, ; rs2499714, 6:34104438, EA, Intron, Non-coding, , ; rs34840245,
6:34844924, EA, intron, Non-coding, eQTL, ; rs6938946, 6:35312920, EA, intron, Non-coding,
eQTL, ; rs6920432, 6:35330885, EA, intergenic, Non-coding, eQTL, ; rs916287, 6:36383081, EA,
Intron, Non-coding, , ; rs12524486, 6:445982, EA, Intergenic, Non-coding, , ; rs9367646, 6:55601574,
EA, intergenic, Non-coding, eQTL, ; rs4573125, 6:5590468, EA, Intron, Non-coding, , ; rs12664535,
6:57312868, EA, intergenic, Non-coding, eQTL, HACER; rs7759743, 6:64676842, EA, Intron, Non-
coding, , ; rs4708114, 6:74065242, EA, Intergenic, Non-coding, , ; rs9353321, 6:85551038, EA,
Intron, Non-coding, , ; rs4707585, 6:89997311, EA, intron, Non-coding, eQTL, ; rs72928038,
6:90267049, EA, Intron, Non-coding, , ; rs2325399, 6:91740418, EA, Intergenic, Non-coding, , ;
rs7810922, 7:101954184, EA, Intron, Non-coding, , ; rs4731532, 7:128932712, EA, intergenic, Non-
coding, eQTL, HACER; rs7808907, 7:128944030, EA, intron, Non-coding, eQTL, ; rs35000415,
7:128945562, EA, intron, Non-coding, eQTL, ; rs12706861, 7:128976528, EA, Intron, Non-coding, , ;
rs11763959, 7:129001852, EA, intron, Non-coding, eQTL, ; rs56272032, 7:150512210, EA, Intron,
Non-coding, , HACER; rs1981601, 7:22162867, EA, Intron, Non-coding, , ; rs73683966, 7:22720392,
EA, Intron, Non-coding, , ; rs4719714, 7:22721094, EA, Intron, Non-coding, , ; rs763616,
7:26744981, EA, intron, Non-coding, eQTL, ; rs10225904, 7:26830269, EA, intron, Non-coding,
eQTL, ; rs3801814, 7:26841683, EA, Intron, Non-coding, , ; rs12531540, 7:28123055, EA, intron,
Non-coding, eQTL, ; rs849142, 7:28146272, EA, Intron, Non-coding, , ; rs17160068, 7:31304379,
EA, Intergenic, Non-coding, , ; rs2109842, 7:31304682, EA, Intergenic, Non-coding, , ; rs2726101,
7:36195490, EA, Intron, Non-coding, , ; rs2083146, 7:38517832, EA, Intron, Non-coding, , ;
rs6945400, 7:51012153, EA, Intron, Non-coding, , ; rs2110258, 7:56126416, EA, intergenic, Non-
coding, eQTL, ; rs73137125, 7:74604624, EA, intergenic, Non-coding, eQTL, ; rs73366469,
7:74619286, EA, intergenic, Non-coding, eQTL, ; rs1922240, 7:87554038, EA, Intron, Non-coding, , ;
rs7831557, 8:10422718, EA, intron, Non-coding, eQTL, ; rs13277113, 8:11491677, EA, Intron, Non-
coding, , ; rs1133950, 8:116771675, EA, Intron, Non-coding, , ; rs867709, 8:120023715, EA, Intron,
Non-coding, , ; rs12156002, 8:128178298, EA, Intergenic, Non-coding, , ; rs11166634, 8:136133917,
EA, Intron, Non-coding, , ; rs34099611, 8:143804458, EA, intron, Non-coding, eQTL, ; rs1193329,
8:19296217, EA, Intergenic, Non-coding, , ; rs351758, 8:28171501, EA, intron, Non-coding, eQTL, ;
rs7829816, 8:55936827, EA, Intron, Non-coding, , ; rs75926644, 8:57395924, EA, Intron, Non-
coding, , ; rs4552888, 8:57403043, EA, Intergenic, Non-coding, , ; rs7823506, 8:59138525, EA,
Intron, Non-coding, , ; rs7826510, 8:5935362, EA, Intergenic, Non-coding, , ; rs17075185, 8:5936095,
EA, Intergenic, Non-coding, , ; rs1966115, 8:78644656, EA, intergenic, Non-coding, eQTL, HACER;
rs12114284, 8:78646206, EA, intergenic, Non-coding, eQTL, HACER; rs2955587, 8:8240557, EA,
intron, Non-coding, eQTL, ; rs13272330, 8:88603626, EA, intron, Non-coding, eQTL, ; rs1039917,
8:8861340, EA, intron, Non-coding, eQTL, ; rs6601327, 8:9538022, EA, intergenic, Non-coding,
eQTL, ; rs11791699, 9:107381080, EA, Intergenic, Non-coding, , ; rs11792269, 9:107382048, EA,
Intergenic, Non-coding, , ; rs6477694, 9:109170062, EA, intergenic, Non-coding, eQTL, ;
rs10118244, 9:114934787, EA, Intron, Non-coding, , ; rs12551731, 9:118200004, EA, Intergenic,
Non-coding, , ; rs10818375, 9:119749136, EA, Intergenic, Non-coding, , ; rs10760059, 9:119752571,
EA, Intergenic, Non-coding, , ; rs2489159, 9:123801631, EA, Intron, Non-coding, , ; rs944333,
9:124324723, EA, Intron, Non-coding, , ; rs3780205, 9:124328889, EA, Intron, Non-coding, , ;
rs1410454, 9:16042195, EA, intron, Non-coding, eQTL, ; rs11788947, 9:266708, EA, Intron, Non-
coding, , ; rs1361925, 9:30085412, EA, Intergenic, Non-coding, , ; rs2380925, 9:34249206, EA,
intron, Non-coding, eQTL, ; rs7872388, 9:4302614, EA, Intron, Non-coding, , ; rs12683801,
9:5272381, EA, Intergenic, Non-coding, , ; rs2804278, 9:582485, EA, Intron, Non-coding, , ;
rs1975197, 9:8846955, EA, Intron, Non-coding, , ; rs874610, 9:98020413, EA, Intron, Non-coding, , ;
rs6478522, 9:98028265, EA, Intron, Non-coding, , ; rs11788118, 9:99575049, EA, Intergenic, Non-
coding, , HACER; rs79399781, 1:172793701, EA, lncRNA, Non-coding RNA, , ; rs2227203,
1:172909883, EA, lncRNA, Non-coding RNA, , ; rs12741962, 1:182287387, EA, lncRNA, Non-
coding RNA, , ; rs12742128, 1:182287457, EA, lncRNA, Non-coding RNA, , ; rs2816317,
1:192567255, EA, lncRNA, Non-coding RNA, eQTL, HACER; rs12096737, 1:226088853, EA,
lncRNA, Non-coding RNA, eQTL, ; rs4948334, 10:59579389, EA, lncRNA, Non-coding RNA,
eQTL, ; rs7121755, 11:115584918, EA, lncRNA, Non-coding RNA, , ; rs560596, 11:28721688, EA,
lncRNA, Non-coding RNA, , ; rs948018, 11:82600972, EA, mIR, Non-coding RNA, , ; rs10850369,
12:114719804, EA, lncRNA, Non-coding RNA, , ; rs12307984, 12:115787613, EA, lncRNA, Non-
coding RNA, , ; rs4242897, 12:8565540, EA, lncRNA, Non-coding RNA, , HACER; rs78506915,
13:44014181, EA, lncRNA, Non-coding RNA, eQTL, ; rs3818246, 14:26775363, EA, lncRNA, Non-
coding RNA, , ; rs882829, 15:40315488, EA, lncRNA, Non-coding RNA, , ; rs11070264,
15:40319660, EA, lncRNA, Non-coding RNA, eQTL, ; rs3743408, 15:80397993, EA, lncRNA, Non-
coding RNA, eQTL, ; rs11635120, 15:97393999, EA, lncRNA, Non-coding RNA, , ; rs905443,
18:77623631, EA, lncRNA, Non-coding RNA, , ; rs16867384, 2:181246479, EA, lncRNA, Non-
coding RNA, , ; rs13410482, 2:181301517, EA, lncRNA, Non-coding RNA, , ; rs7601533,
2:52091336, EA, lncRNA, Non-coding RNA, , ; rs115291397, 2:60832908, EA, lncRNA, Non-coding
RNA, , ; rs2823288, 21:15448569, EA, lncRNA, Non-coding RNA, eQTL, ; rs78712140,
3:101996985, EA, lncRNA, Non-coding RNA, , ; rs564976, 3:160011272, EA, lncRNA, Non-coding
RNA, , ; rs1435649, 3:177877150, EA, lncRNA, Non-coding RNA, , ; rs4375990, 3:24733421, EA,
lncRNA, Non-coding RNA, , ; rs463149, 3:6780173, EA, lncRNA, Non-coding RNA, , ; rs6857709,
4:64918527, EA, lncRNA, Non-coding RNA, , ; rs461193, 5:1368882, EA, lncRNA, Non-coding
RNA, , ; rs4921317, 5:159111269, EA, lncRNA, Non-coding RNA, , ; rs6869688, 5:159456019, EA,
lncRNA, Non-coding RNA, , ; rs7720046, 5:159457527, EA, lncRNA, Non-coding RNA, , ;
rs2431697, 5:160452971, EA, lncRNA, Non-coding RNA, , HACER; rs888656, 5:160476863, EA,
mIR, Non-coding RNA, , ; rs9283855, 6:169378334, EA, lncRNA, Non-coding RNA, , ; rs760583,
6:169380665, EA, lncRNA, Non-coding RNA, , ; rs2456449, 8:127180736, EA, lncRNA, Non-coding
RNA, eQTL, ; rs6651252, 8:128554935, EA, lncRNA, Non-coding RNA, , HACER; rs2026995,
9:129337112, EA, lncRNA, Non-coding RNA, , ; rs816685, 9:95821144, EA, lncRNA, Non-coding
RNA, , ; rs10819689, 9:99637981, EA, lncRNA, Non-coding RNA, , ; rs9651118, 1:11802157, EA,
promoter, Regulatory, , ; rs45444697, 1:155062156, EA, promoter, Regulatory, , ; rs9887904,
1:159008806, EA, promoter, Regulatory, , ; rs1061511, 1:160219710, EA, PFR, Regulatory, eQTL, ;
rs12129787, 1:161522797, EA, PFR, Regulatory, eQTL, ; rs164178, 1:162387863, EA, PFR,
Regulatory, , ; rs17484292, 1:183330915, EA, PFR, Regulatory, , ; rs41263646, 1:183597997, EA,
CTCF binding site, Regulatory, eQTL, HACER; rs1539414, 1:197774376, EA, promoter, Regulatory,
eQTL, ; rs17610618, 1:198640778, EA, promoter, Regulatory, , ; rs6680666, 1:198960312, EA, PFR,
Regulatory, , ; rs1340237, 1:203227153, EA, PFR, Regulatory, , ; rs7533588, 1:227755141, EA, PFR,
Regulatory, eQTL, ; rs4648892, 1:23393692, EA, PFR, Regulatory, , GeneHancer; rs72952888,
1:91948449, EA, promoter, Regulatory, eQTL, ; rs11578098, 1:92653853, EA, CTCF binding site,
Regulatory, , ; rs771459, 1:93355031, EA, PFR, Regulatory, eQTL, ; rs117297579, 10:58065900, EA,
CTCF binding site, Regulatory, , ; rs12722558, 10:6028313, EA, PFR, Regulatory, , ; rs10905718,
10:6072893, EA, promoter, Regulatory, , GeneHancer; rs2688608, 10:73898591, EA, PFR,
Regulatory, eQTL, ; rs1473176, 10:99563915, EA, promoter, Regulatory, , ; rs2122775,
11:103528536, EA, PFR, Regulatory, , ; rs501089, 11:111631755, EA, PFR, Regulatory, eQTL, ;
rs513425, 11:111634137, EA, PFR, Regulatory, , GeneHancer; rs662799, 11:116792991, EA, OCR,
Regulatory, eQTL, ; rs600751, 11:126189565, EA, TFBS, Regulatory, eQTL, GeneHancer;
rs73029013, 11:128447862, EA, PFR, Regulatory, , ; rs4936059, 11:128632601, EA, CTCF binding
site, Regulatory, , ; rs1647960, 11:131051857, EA, Enhancer, Regulatory, , ; rs2732549, 11:35066852,
EA, PFR, Regulatory, eQTL, HACER; rs6598011, 11:577809, EA, promoter, Regulatory, eQTL, ;
rs11231814, 11:64542178, EA, OCR, Regulatory, , ; rs2298455, 11:71999432, EA, promoter,
Regulatory, , GeneHancer; rs653178, 12:111569952, EA, PFR, Regulatory, eQTL, ; rs2283293,
12:2311441, EA, PFR, Regulatory, , ; rs1565123, 12:24665378, EA, PFR, Regulatory, , ; rs2456973,
12:56023144, EA, promoter, Regulatory, , ; rs6581430, 12:61766297, EA, Enhancer, Regulatory, , ;
rs10506577, 12:69973634, EA, CTCF binding site, Regulatory, , ; rs10492224, 12:96167963, EA,
PFR, Regulatory, , ; rs117836922, 12:9745556, EA, Enhancer, Regulatory, , ; rs2244272,
15:52868417, EA, Enhancer, Regulatory, eQTL, ; rs4777427, 15:71637548, EA, CTCF binding site,
Regulatory, , ; rs1351161, 15:73724562, EA, promoter, Regulatory, , ; rs243323, 16:11267345, EA,
TFBS, Regulatory, eQTL, ; rs72799341, 16:30925422, EA, promoter, Regulatory, eQTL, ;
rs34572943, 16:31261032, EA, promoter, Regulatory, , ; rs6499034, 16:65749038, EA, PFR,
Regulatory, , ; rs1453560, 17:39867188, EA, TFBS, Regulatory, , GeneHancer; rs962885,
17:45858265, EA, TFBS, Regulatory, , ; rs3829578, 17:55304149, EA, CTCF binding site,
Regulatory, , ; rs4796445, 17:6232520, EA, CTCF binding site, Regulatory, , ; rs9894206
17:75364073, EA, Enhancer, Regulatory, eQTL, ; rs1463485, 17:75855710, EA, Promoter,
Regulatory, eQTL, ; rs12946196, 17:77811828, EA, PFR, Regulatory, , ; rs11658698, 17:80544527,
EA, Promoter, Regulatory, eQTL, ; rs62097857, 18:12857759, EA, PFR, Regulatory, , GeneHancer;
rs9965182, 18:58110469, EA, Enhancer, Regulatory, , GeneHancer; rs62136099, 19:46344644, EA,
Enhancer, Regulatory, , ; rs4802307, 19:46346549, EA, promoter, Regulatory, eQTL, GeneHancer;
rs10419198, 19:49534760, EA, CTCF binding site, Regulatory, eQTL, ; rs169080, 19:4980853, EA,
CTCF binding site, Regulatory, , ; rs11681903, 2:118116060, EA, promoter, Regulatory, , ; rs936126,
2:128366761, EA, Enhancer, Regulatory, , GeneHancer; rs2111485, 2:162254026, EA, Enhancer,
Regulatory, , ; rs13023380, 2:162297853, EA, TFBS, Regulatory, , ; rs13383488, 2:163755144, EA,
TFBS, Regulatory, , ; rs6715106, 2:191048308, EA, Enhancer, Regulatory, , GeneHancer, rs7582694,
2:191105394, EA, OCR, Regulatory, , ; rs3731570, 2:197453059, EA, promoter, Regulatory, ,
GeneHancer; rs13384308, 2:205931103, EA, PFR, Regulatory, , ; rs10202434, 2:237342392, EA,
PFR, Regulatory, , ; rs13026988, 2:241467764, EA, CTCF binding site, Regulatory, , ; rs13425999,
2:33477136, EA, promoter, Regulatory, , ; rs6705304, 2:43369607, EA, PFR, Regulatory, , ; rs653215,
20:13307724, EA, TFBS, Regulatory, , GeneHancer; rs6131014, 20:46108744, EA, TFBS,
Regulatory, eQTL, GeneHancer; rs913678, 20:50338887, EA, CTCF binding site, Regulatory, ,
HACER; rs4811174, 20:51431596, EA, Enhancer, Regulatory, , ; rs2235947, 20:8648049, EA, PFR,
Regulatory, , ; rs7280984, 21:36859568, EA, PFR, Regulatory, eQTL, ; rs11089629, 22:21604583,
EA, PFR, Regulatory, eQTL, ; rs17483887, 22:28027856, EA, PFR, Regulatory, , ; rs16991802,
22:33116757, EA, Enhancer, Regulatory, , ; rs4482697, 3:121817650, EA, Enhancer, Regulatory,
eQTL, ; rs7633180, 3:18657320, EA, PFR, Regulatory, , ; rs3773377, 3:377460, EA, OCR,
Regulatory, , ; rs180977001, 3:58332737, EA, TFBS, Regulatory, , GeneHancer; rs6445975,
3:58384450, EA, PFR, Regulatory, eQTL, ; rs11130633, 3:58385065, EA, PFR, Regulatory, eQTL, ;
rs11705804, 3:66741261, EA, OCR, Regulatory, , ; rs1499077, 3:7382653, EA, OCR, Regulatory, ,
GeneHancer; rs13126505, 4:101944147, EA, PFR, Regulatory, , ; rs4515165, 4:113812714, EA, PFR,
Regulatory, , ; rs4690055, 4:2746936, EA, PFR, Regulatory, eQTL, ; rs1565901, 4:61541609, EA,
TFBS, Regulatory, , GeneHancer; rs4610334, 4:7256775, EA, Enhancer, Regulatory, , ; rs6449173,
4:9964481, EA, Enhancer, Regulatory, , ; rs72768698, 5:103255478, EA, PFR, Regulatory, , ;
rs6595788, 5:127651369, EA, PFR, Regulatory, , ; rs73286503, 5:134211525, EA, PFR, Regulatory, ,
; rs7708392, 5:151077924, EA, promoter, Regulatory, , ; rs6889239, 5:151078210, EA, promoter,
Regulatory, , GeneHancer; rs57095329, 5:160467840, EA, TFBS, Regulatory, , GeneHancer;
rs1864975, 5:167460624, EA, OCR, Regulatory, , ; rs59494035, 5:174082936, EA, CTCF binding
site, Regulatory, , ; rs4869426, 5:35825471, EA, promoter, Regulatory, , ; rs11134275, 5:8024710,
EA, PFR, Regulatory, , ; rs11738358, 5:96722187, EA, PFR, Regulatory, eQTL, ; rs72941674,
6:106074335, EA, Enhancer, Regulatory, , ; rs6923608, 6:106089915, EA, promoter, Regulatory, , ;
rs4946810, 6:107099066, EA, PFR, Regulatory, , GeneHancer; rs352834, 6:109202618, EA, CTCF
binding site, Regulatory, eQTL, HACER; rs1413753, 6:130829364, EA, Promoter, Regulatory, eQTL,
; rs17779870, 6:137835288, EA, Enhancer, Regulatory, , ; rs5029939, 6:137874586, EA, promoter,
Regulatory, , GeneHancer; rs6923307, 6:159044592, EA, promoter, Regulatory, eQTL, ; rs2057016,
6:22656099, EA, Enhancer, Regulatory, , ; rs9462027, 6:34829464, EA, OCR, Regulatory, EQTL, ;
rs7764323, 6:36378063, EA, PFR, Regulatory, eQTL, ; rs1983891, 6:41568689, EA, TFBS,
Regulatory, eQTL, ; rs79558495, 7:100767812, EA, CTCF binding site, Regulatory, , ; rs10486135,
7:11488873, EA, PFR, Regulatory, , ; rs3807307, 7:128939148, EA, Promoter, Regulatory, eQTL, ;
rs10952261, 7:150481940, EA, Promoter, Regulatory, eQTL, ; rs6950918, 7:155141302, EA, PFR,
Regulatory, , ; rs12666575, 7:1964786, EA, Enhancer, Regulatory, , ; rs702814, 7:28133113, EA,
PFR, Regulatory, eQTL, ; rs4917014, 7:50266267, EA, promoter, Regulatory, , HACER; rs6945305,
7:8136157, EA, OCR, Regulatory, , ; rs7812879, 8:11482672, EA, PFR, Regulatory, eQTL, HACER;
rs11998187, 8:13252295, EA, PFR, Regulatory, , ; rs6980682, 8:133375064, EA, PFR, Regulatory,
eQTL, ; rs13252899, 8:144350943, EA, PFR, Regulatory, eQTL, ; rs4469464, 8:23694138, EA, PFR,
Regulatory, , ; rs11136577, 8:3103568, EA, Enhancer, Regulatory, , ; rs4298479, 8:58260802, EA,
OCR, Regulatory, , ; rs7821169, 8:58275241, EA, OCR, Regulatory, , ; rs7011788, 8:60981364, EA,
PFR, Regulatory, , HACER; rs452999, 9:133967406, EA, PFR, Regulatory, , GeneHancer;
rs16935357, 9:38708762, EA, OCR, Regulatory, , ; rs10868841, 9:70503748, EA, PFR, Regulatory, , ;
;

TABLE 14
List of all Immunochip SNP-predicted genes by discovery method/source
and ancestral designation. Listed by: Gene, Source, Ancestry;

ADAMTSL3, C-Gene, AsA; ANOS1, C-Gene, AsA; CAST, C-Gene, AsA; CBLIF, C-Gene, AsA;
CCDC116, C-Gene, AsA; CCDC8, C-Gene, AsA; CCL22, C-Gene, AsA; CTNS, C-Gene, AsA;
ERAP1, C-Gene, AsA; IGFBP3, C-Gene, AsA; IGSF3, C-Gene, AsA; IL18R1, C-Gene, AsA; IRAK1,
C-Gene, AsA; MYO9B, C-Gene, AsA; NCF2, C-Gene, AsA; NEB, C-Gene, AsA; NLGN4X, C-Gene,
AsA; OAS3, C-Gene, AsA; OXA1L, C-Gene, AsA; RASGRF2, C-Gene, AsA; REST, C-Gene, AsA;
SLC45A2, C-Gene, AsA; SPINK8, C-Gene, AsA; TNFRSF13B, C-Gene, AsA; TRPV1, C-Gene,
AsA; VWF, C-Gene, AsA; WDFY4, C-Gene, AsA; RP11-284E5.1, E-Gene, AsA; RP11-63G10.4, E-
Gene, AsA; RP11-122G18.11, E-Gene, AsA; RP11-263J14.1, E-Gene, AsA; RASSF1-AS1, E-Gene,
AsA; AC010872.1, E-Gene, AsA; CTD-2013N17.6, E-Gene, AsA; RP3-333H23.9, E-Gene, AsA;
XX-FW83563B9.5, E-Gene, AsA; RP11-196G11.3, E-Gene, AsA; RP11-331F4.5, E-Gene, AsA;
RP11-385H1.1, E-Gene, AsA; AL022393.9, E-Gene, AsA; CTC-463A16.1, E-Gene, AsA; DNM1P41,
E-Gene, AsA; CTD-3222D19.9, E-Gene, AsA; RP11-44F14.6, E-Gene, AsA; RP1-102K2.9, E-Gene,
AsA; RP11-215P8.2, E-Gene, AsA; RP11-1072A3.3, E-Gene, AsA; RP3-394A18.1, E-Gene, AsA;
AC002310.14, E-Gene, AsA; AP000560.3, E-Gene, AsA; RP11-458J1.1, E-Gene, AsA; HIST1H3H,
E-Gene, AsA; CTD-2318O12.1, E-Gene, AsA; RP11-95I16.6, E-Gene, AsA; PMS2P2, E-Gene, AsA;
SSTR3, E-Gene, AsA; RP11-196G11.5, E-Gene, AsA; F8A1, E-Gene, AsA; STAG3L2, E-Gene,
AsA; GTF2IP1, E-Gene, AsA; RP11-290H9.5, E-Gene, AsA; HIST1H2BB, E-Gene, AsA; HIST1H4I,
E-Gene, AsA; IKBKGP1, E-Gene, AsA; CCL15, E-Gene, AsA; RP11-1072A3.4, E-Gene, AsA;
RP11-309L24.10, E-Gene, AsA; RP1-292L20.3, E-Gene, AsA; HIST1H3E, E-Gene, AsA; RCC1L, E-
Gene, AsA; H2AFB1, E-Gene, AsA; GATSL2, E-Gene, AsA; LCA10, E-Gene, AsA; LINC02019, E-
Gene, AsA; KB-1440D3.14, E-Gene, AsA; RP11-448A19.1, E-Gene, AsA; RP11-493E12.2, E-Gene,
AsA; RP11-455J20.3, E-Gene, AsA; RP11-479G22.8, E-Gene, AsA; RP11-336K24.12, E-Gene, AsA;
RP11-647K16.1, E-Gene, AsA; RP11-47A8.5, E-Gene, AsA; U91328.22, E-Gene, AsA; RP11-
390E23.6, E-Gene, AsA; CFAP206, E-Gene, AsA; RP11-981G7.6, E-Gene, AsA; U91328.19, E-
Gene, AsA; POM121C, E-Gene, AsA; RP5-1112D6.8, E-Gene, AsA; KMT2B, E-Gene, AsA; RP11-
140K17.3, E-Gene, AsA; RP11-375N15.2, E-Gene, AsA; GS1-393G12.14, E-Gene, AsA; CTD-
2260A17.3, E-Gene, AsA; CTD-2376I4.2, E-Gene, AsA; CTD-3025N20.3, E-Gene, AsA; RP1-
313I6.12, E-Gene, AsA; RP11-188P20.3, E-Gene, AsA; RP11-493E12.1, E-Gene, AsA; RP5-
894D12.5, E-Gene, AsA; RP5-1112D6.7, E-Gene, AsA; RP1-153G14.4, E-Gene, AsA; RP11-
110I1.14, E-Gene, AsA; NAMA, E-Gene, AsA; RP11-355B11.2, E-Gene, AsA; RP11-327F22.6, E-
Gene, AsA; RP11-380L11.4, E-Gene, AsA; RP11-148O21.6, E-Gene, AsA; RP11-738E22.3, E-Gene,
AsA; RP6-91H8.3, E-Gene, AsA; AF131215.9, E-Gene, AsA; RP11-542M13.2, E-Gene, AsA; CTB-
174O21.2, E-Gene, AsA; CTD-3032J10.3, E-Gene, AsA; CTC-429P9.3, E-Gene, AsA; CTC-429P9.2,
E-Gene, AsA; LIN37, E-Gene, AsA; FDX2, E-Gene, AsA; CTD-2554C21.2, E-Gene, AsA; CTD-
2162K18.3, E-Gene, AsA; NAGPA-AS1, E-Gene, AsA; AC002398.13, E-Gene, AsA; RP11-
138P22.1, E-Gene, AsA; RASSF5, E-Gene, AsA; IKBKE, E-Gene, AsA; THCAT158, E-Gene, AsA;
HNRNPCP4, E-Gene, AsA; RP11-485G7.5, E-Gene, AsA; RP11-396B14.2, E-Gene, AsA; RP11-
876N24.5, E-Gene, AsA; GTF2I, E-Gene, AsA; RP11-196G11.4, E-Gene, AsA; RP11-876N24.4, E-
Gene, AsA; AC144831.1, E-Gene, AsA; RP11-252K23.2, E-Gene, AsA; RP11-327F22.1, E-Gene,
AsA; TMEM249, E-Gene, AsA; RP11-981G7.1, E-Gene, AsA; RP11-120K18.3, E-Gene, AsA; RP11-
534L20.5, E-Gene, AsA; RP11-731J8.2, E-Gene, AsA; RP11-196G11.2, E-Gene, AsA; RP11-
367F23.2, E-Gene, AsA; RP13-735L24.1, E-Gene, AsA; RP11-384K6.6, E-Gene, AsA; HSPB9, E-
Gene, AsA; RP11-388M20.6, E-Gene, AsA; RP11-973H7.1, E-Gene, AsA; SSSCA1-AS1, E-Gene,
AsA; RP11-254F7.2, E-Gene, AsA; CTD-2270L9.2, E-Gene, AsA; RP11-182J1.13, E-Gene, AsA;
LINC00933, E-Gene, AsA; CSPG4P11, E-Gene, AsA; RP11-299H22.1, E-Gene, AsA; RP11-
671M22.4, E-Gene, AsA; RP11-244F12.3, E-Gene, AsA; RP11-321G12.1, E-Gene, AsA; EFTUD1P1,
E-Gene, AsA; RP1-261D10.2, E-Gene, AsA; RP11-507K2.2, E-Gene, AsA; SRP54-AS1, E-Gene,
AsA; PTTG4P, E-Gene, AsA; RTEL1, E-Gene, AsA; LINC01481, E-Gene, AsA; RP3-473L9.4, E-
Gene, AsA; RP1-71H24.1, E-Gene, AsA; RP11-320P7.2, E-Gene, AsA; RP11-705C15.3, E-Gene,
AsA; RP11-525E9.1, E-Gene, AsA; AP000462.3, E-Gene, AsA; RP11-500M8.6, E-Gene, AsA; RP11-
599J14.2, E-Gene, AsA; CLEC12B, E-Gene, AsA; RP11-705C15.2, E-Gene, AsA; RP11-118B22.4,
E-Gene, AsA; HMBS, E-Gene, AsA; RP11-324E6.6, E-Gene, AsA; RP13-942N8.1, E-Gene, AsA;
A2MP1, E-Gene, AsA; IFNG-AS1, E-Gene, AsA; RAB44, E-Gene, AsA; RP11-770G2.2, E-Gene,
AsA; RP11-351I21.6, E-Gene, AsA; RP4-607I7.1, E-Gene, AsA; RP11-148O21.4, E-Gene, AsA;
RP11-535A19.2, E-Gene, AsA; AC145124.2, E-Gene, AsA; HCAR3, E-Gene, AsA; C8orf49, E-
Gene, AsA; C6orf3, E-Gene, AsA; RP11-148O21.2, E-Gene, AsA; AF131215.2, E-Gene, AsA;
TMPRSS4-AS1, E-Gene, AsA; RP11-481A20.4, E-Gene, AsA; EID3, E-Gene, AsA; AP000442.1, E-
Gene, AsA; RP11-110I1.12, E-Gene, AsA; OVOL1-AS1, E-Gene, AsA; AF131216.5, E-Gene, AsA;
KRTAP5-9, E-Gene, AsA; AF131215.3, E-Gene, AsA; DEFB109P3, E-Gene, AsA; MPV17L2, E-
Gene, AsA; CTD-2530H12.2, E-Gene, AsA; RP11-252C15.1, E-Gene, AsA; RP11-148O21.3, E-
Gene, AsA; ENPP7P12, E-Gene, AsA; SIGLEC12, E-Gene, AsA; RP11-481A20.10, E-Gene, AsA;
RP11-522I20.3, E-Gene, AsA; AP5B1, E-Gene, AsA; RP11-351I21.7, E-Gene, AsA; RP11-10A14.3,
E-Gene, AsA; RP11-131N11.4, E-Gene, AsA; RP11-115J16.2, E-Gene, AsA; PCDHGB7, E-Gene,
AsA; LYN, E-Gene, AsA; IGHEP2, E-Gene, AsA; ZNF260, E-Gene, AsA; CLDN23, E-Gene, AsA;
RP11-115J16.3, E-Gene, AsA; OSGEPL1-AS1, E-Gene, AsA; LINC01099, E-Gene, AsA; RP11-
369K16.1, E-Gene, AsA; FAM90A25P, E-Gene, AsA; RP11-10L12.2, E-Gene, AsA; LINC00491, E-
Gene, AsA; CTA-963H5.5, E-Gene, AsA; DNAH10OS, E-Gene, AsA; LINC00603, E-Gene, AsA;
TMEM158, E-Gene, AsA; ALGIL11P, E-Gene, AsA; AC011330.5, E-Gene, AsA; CTD-2227C6.2, E-
Gene, AsA; CTD-2203K17.1, E-Gene, AsA; HAUS5, E-Gene, AsA; ZNF436-AS1, E-Gene, AsA;
KRT8P46, E-Gene, AsA; CTD-2260A17.1, E-Gene, AsA; RP11-10A14.5, E-Gene, AsA; ABHD14A,
E-Gene, AsA; RP11-10L12.4, E-Gene, AsA; RP11-158I9.5, E-Gene, AsA; FCGR2C, E-Gene, AsA;
RP4-800G7.2, E-Gene, AsA; RN7SL130P, E-Gene, AsA; P2RY11, E-Gene, AsA; LCE3C, E-Gene,
AsA; TNFRSF6B, E-Gene, AsA; NAT6, E-Gene, AsA; MCCC1-AS1, E-Gene, AsA; GSTA9P, E-
Gene, AsA; RP11-469J4.3, E-Gene, AsA; NPPA-AS1, E-Gene, AsA; RPL10P7, E-Gene, AsA;
RPSAP52, E-Gene, AsA; GUSBP2, E-Gene, AsA; KRTAP5-8, E-Gene, AsA; RP11-147I3.1, E-Gene,
AsA; ARHGEF25, E-Gene, AsA; RPS6P25, E-Gene, AsA; RPL5P23, E-Gene, AsA; RP1-93H18.1, E-
Gene, AsA; MEIKIN, E-Gene, AsA; AC007405.6, E-Gene, AsA; AC068196.1, E-Gene, AsA; RP11-
305M3.2, E-Gene, AsA; SIRPG-AS1, E-Gene, AsA; P4HA2-AS1, E-Gene, AsA; C2orf74, E-Gene,
AsA; HLCS-IT1, E-Gene, AsA; RP11-356I2.4, E-Gene, AsA; RP11-280O1.2, E-Gene, AsA; RP11-
554F20.1, E-Gene, AsA; RP11-165J3.5, E-Gene, AsA; CDKN2AIPNL, E-Gene, AsA; MCFD2P1, E-
Gene, AsA; RP11-78B10.2, E-Gene, AsA; RP11-446E9.1, E-Gene, AsA; GSN-AS1, E-Gene, AsA;
RP11-282O18.3, E-Gene, AsA; DNM1P51, E-Gene, AsA; RP11-575L7.8, E-Gene, AsA; ZSCAN31,
E-Gene, AsA; RP11-140A10.3, E-Gene, AsA; MIR155HG, E-Gene, AsA; LINC01239, E-Gene, AsA;
LINC01135, E-Gene, AsA; RP11-445L6.3, E-Gene, AsA; SNRK-AS1, E-Gene, AsA; MAPKAPK5-
AS1, E-Gene, AsA; ACBD3-AS1, E-Gene, AsA; PINLYP, E-Gene, AsA; MAGI2-AS3, E-Gene,
AsA; JAZF1-AS1, E-Gene, AsA; AC116366.6, E-Gene, AsA; RP11-168O16.2, E-Gene, AsA;
LINC01360, E-Gene, AsA; HIST1H2BN, E-Gene, AsA; AC093388.3, E-Gene, AsA; RPL10P19, E-
Gene, AsA; RP11-153K11.3, E-Gene, AsA; RP11-500B12.1, E-Gene, AsA; HOTAIRM1, E-Gene,
AsA; MROH3P, E-Gene, AsA; AC034220.3, E-Gene, AsA; AC067959.1, E-Gene, AsA; RP11-
544M22.3, E-Gene, AsA; LYRM9, E-Gene, AsA; RP11-196G11.6, E-Gene, AsA; RP5-1011O1.2, E-
Gene, AsA; RP4-730K3.3, E-Gene, AsA; IGHV3-43, E-Gene, AsA; TMA7, E-Gene, AsA; DARS-
AS1, E-Gene, AsA; RP11-686D16.1, E-Gene, AsA; DLEU2, E-Gene, AsA; RP11-187A9.3, E-Gene,
AsA; LINC00689, E-Gene, AsA; FBXW4P1, E-Gene, AsA; GPAA1P2, E-Gene, AsA; GOLGA6L5P,
E-Gene, AsA; AC004980.9, E-Gene, AsA; RP5-1112D6.4, E-Gene, AsA; LRRC37A15P, E-Gene,
AsA; MRPS18AP1, E-Gene, AsA; RP11-142M10.2, E-Gene, AsA; SPINK8, E-Gene, AsA;
KRT8P12, E-Gene, AsA; RP11-168O16.1, E-Gene, AsA; VDAC1P8, E-Gene, AsA; MRPL45P2, E-
Gene, AsA; FCF1P2, E-Gene, AsA; RP11-563N6.6, E-Gene, AsA; RP11-367F23.1, E-Gene, AsA;
MTND1P11, E-Gene, AsA; TOP3BP1, E-Gene, AsA; RP11-130C19.3, E-Gene, AsA; FAM66A, E-
Gene, AsA; RP11-165J3.6, E-Gene, AsA; RP1-167A14.2, E-Gene, AsA; LINC00376, E-Gene, AsA;
RP11-162D16.2, E-Gene, AsA; SUB1P1, E-Gene, AsA; CNN2P1, E-Gene, AsA; HORMAD2-AS1,
E-Gene, AsA; RP5-956O18.2, E-Gene, AsA; PDZPH1P, E-Gene, AsA; SRRM5, E-Gene, AsA;
PSMD5-AS1, E-Gene, AsA; LINC01535, E-Gene, AsA; UPK1A-AS1, E-Gene, AsA; ADAM1B, E-
Gene, AsA; ZNF192P1, E-Gene, AsA; RP1-151B14.9, E-Gene, AsA; AP4B1-AS1, E-Gene, AsA;
AF277315.13, E-Gene, AsA; LINC01534, E-Gene, AsA; LINC01529, E-Gene, AsA; AC016582.2, E-
Gene, AsA; LINC01980, E-Gene, AsA; LINC01703, E-Gene, AsA; HSPA7, E-Gene, AsA;
GOLGA2P7, E-Gene, AsA; LINC01766, E-Gene, AsA; LINC00240, E-Gene, AsA; KRT18P39, E-
Gene, AsA; AP000704.5, E-Gene, AsA; CETN4P, E-Gene, AsA; AC025165.8, E-Gene, AsA;
ZMIZ1-AS1, E-Gene, AsA; TMLHE-AS1, E-Gene, AsA; RP11-10L12.1, E-Gene, AsA; RPS23P10,
E-Gene, AsA; CCNT2-AS1, E-Gene, AsA; RP11-728K20.1, E-Gene, AsA; ENTPD3-AS1, E-Gene,
AsA; NSUN5P1, E-Gene, AsA; CKMT1A, E-Gene, AsA; FADS3, E-Gene, AsA; OR1F12, E-Gene,
AsA; FCF1P5, E-Gene, AsA; ZGLP1, E-Gene, AsA; ZSCAN12P1, E-Gene, AsA; ZNF602P, E-Gene,
AsA; TDRD15, E-Gene, AsA; ZNF192P2, E-Gene, AsA; IQCB2P, E-Gene, AsA; FNIP1, E-Gene,
AsA; CCDC7, E-Gene, AsA; GPR89P, E-Gene, AsA; ZNF603P, E-Gene, AsA; C1orf167, E-Gene,
AsA; CD27-AS1, E-Gene, AsA; DDX12P, E-Gene, AsA; OOSP1, E-Gene, AsA; RP11-726G1.1, E-
Gene, AsA; KRT8P26, E-Gene, AsA; FAM185BP, E-Gene, AsA; AS3MT, E-Gene, AsA;
AC004980.10, E-Gene, AsA; RPL13P2, E-Gene, AsA; TREX1, E-Gene, AsA; NCKIPSD, E-Gene,
AsA; SIPA1, E-Gene, AsA; DNLZ, E-Gene, AsA; FGFR1OP, E-Gene, AsA; AC016747.3, E-Gene,
AsA; IGHV3-20, E-Gene, AsA; IGHD3-3, E-Gene, AsA; GPX3, E-Gene, AsA; SNORD83A, E-Gene,
AsA; DOK6, E-Gene, AsA; DEFB134, E-Gene, AsA; CYS1, E-Gene, AsA; CATSPER2P1, E-Gene,
AsA; STAG3L1, E-Gene, AsA; PDE7A, E-Gene, AsA; ZNF783, E-Gene, AsA; FAM216A, E-Gene,
AsA; TCTN1, E-Gene, AsA; PNMAL2, E-Gene, AsA; ATXN2, E-Gene, AsA; ZNF204P, E-Gene,
AsA; TRIM27, E-Gene, AsA; ACOXL-AS1, E-Gene, AsA; TCEA3, E-Gene, AsA; C10orf128, E-
Gene, AsA; GGTA1P, E-Gene, AsA; RUFY2, E-Gene, AsA; TRAF3IP1, E-Gene, AsA; C6orf163, E-
Gene, AsA; C1orf53, E-Gene, AsA; STUM, E-Gene, AsA; INF2, E-Gene, AsA; LINC00449, E-Gene,
AsA; TBKBP1, E-Gene, AsA; L1CAM, E-Gene, AsA; C1orf68, E-Gene, AsA; SCAMP5, E-Gene,
AsA; RPL10A, E-Gene, AsA; PLXNB3, E-Gene, AsA; IPO9, E-Gene, AsA; TLK1, E-Gene, AsA;
C2CD4A, E-Gene, AsA; CNGA1, E-Gene, AsA; UVRAG, E-Gene, AsA; RP11-544M22.1, E-Gene,
AsA; ZKSCAN8, E-Gene, AsA; TMEM116, E-Gene, AsA; ZNF607, E-Gene, AsA; TMEM229B, E-
Gene, AsA; HIST1H2BK, E-Gene, AsA; RP1-253P7.4, E-Gene, AsA; PSAP, E-Gene, AsA; NMB, E-
Gene, AsA; C5orf42, E-Gene, AsA; SIPA1L1, E-Gene, AsA; C5orf56, E-Gene, AsA; SLC28A3, E-
Gene, AsA; STMN3, E-Gene, AsA; SHPK, E-Gene, AsA; SLC22A5, E-Gene, AsA; TRIM33, E-
Gene, AsA; ZNF165, E-Gene, AsA; C6orf141, E-Gene, AsA; HIST1H3J, E-Gene, AsA; PCNX3, E-
Gene, AsA; ZNF300P1, E-Gene, AsA; ZSCAN26, E-Gene, AsA; LAGE3, E-Gene, AsA; HCAR1, E-
Gene, AsA; C6orf106, E-Gene, AsA; ZSCAN16, E-Gene, AsA; TRPV1, E-Gene, AsA; ZNF781, E-
Gene, AsA; GTF2IRD2, E-Gene, AsA; CCDC189, E-Gene, AsA; NEMP2, E-Gene, AsA; ZKSCAN3,
E-Gene, AsA; ALKAL2, E-Gene, AsA; TSPYL1, E-Gene, AsA; UBE2Q2P1, E-Gene, AsA;
FAM120AOS, E-Gene, AsA; RPL14, E-Gene, AsA; FAM26F, E-Gene, AsA; BCL2L15, E-Gene,
AsA; JAKMIP3, E-Gene, AsA; LRRIQ4, E-Gene, AsA; PRSS45, E-Gene, AsA; ZSCAN23, E-Gene,
AsA; HIST1H1C, E-Gene, AsA; OR2B7P, E-Gene, AsA; ZKSCAN4, E-Gene, AsA; TLR5, E-Gene,
AsA; HIST1H1T, E-Gene, AsA; LCE4A, E-Gene, AsA; HYAL3, E-Gene, AsA; BCR, E-Gene, AsA;
DTX2P1, E-Gene, AsA; BTN3A2, E-Gene, AsA; PPP1CC, E-Gene, AsA; POLR1D, E-Gene, AsA;
TMLHE, E-Gene, AsA; TRIM69, E-Gene, AsA; SLC52A2, E-Gene, AsA; UBE2L3, E-Gene, AsA;
BRCC3, E-Gene, AsA; TCN2, E-Gene, AsA; SIGIRR, E-Gene, AsA; MIXL1, E-Gene, AsA;
ADSSL1, E-Gene, AsA; F8, E-Gene, AsA; SIVA1, E-Gene, AsA; RBM43, E-Gene, AsA; B3GALT5-
AS1, E-Gene, AsA; OSBP2, E-Gene, AsA; CDCA2, E-Gene, AsA; SPDYE12P, E-Gene, AsA;
FAM167A-AS1, E-Gene, AsA; ZFP1, E-Gene, AsA; ACTRT3, E-Gene, AsA; PKP3, E-Gene, AsA;
PRKD1, E-Gene, AsA; CLECL1, E-Gene, AsA; TACSTD2, E-Gene, AsA; IRAK1, E-Gene, AsA;
GOLGA6L4, E-Gene, AsA; NIPSNAP1, E-Gene, AsA; UQCR10, E-Gene, AsA; KMT5A, E-Gene,
AsA; LIN9, E-Gene, AsA; FAM162B, E-Gene, AsA; B3GALT5, E-Gene, AsA; NEB, E-Gene, AsA;
IGSF5, E-Gene, AsA; HMGN4, E-Gene, AsA; ACBD3, E-Gene, AsA; TRAK1, E-Gene, AsA;
OR2B8P, E-Gene, AsA; PDSS1P1, E-Gene, AsA; FBXL6, E-Gene, AsA; B4GALNT4, E-Gene, AsA;
ARL6IP4, E-Gene, AsA; TMEM259, E-Gene, AsA; PNMAL1, E-Gene, AsA; ADO, E-Gene, AsA;
HKR1, E-Gene, AsA; ZNF322, E-Gene, AsA; FKRP, E-Gene, AsA; SSR4, E-Gene, AsA; S1PR5, E-
Gene, AsA; ARHGAP45, E-Gene, AsA; TH, E-Gene, AsA; ATOH7, E-Gene, AsA; LACCI, E-Gene,
AsA; LSMEM2, E-Gene, AsA; HTR1D, E-Gene, AsA; EIF3J-AS1, E-Gene, AsA; WSCD1, E-Gene,
AsA; CALR, E-Gene, AsA; ZNF664, E-Gene, AsA; FARSA, E-Gene, AsA; RMI1, E-Gene, AsA;
TAF7, E-Gene, AsA; RFLNA, E-Gene, AsA; EFCAB13, E-Gene, AsA; TRIM73, E-Gene, AsA;
RPP25, E-Gene, AsA; P4HTM, E-Gene, AsA; WDR6, E-Gene, AsA; PRSS36, E-Gene, AsA;
LCORL, E-Gene, AsA; DALRD3, E-Gene, AsA; FAM26E, E-Gene, AsA; ADAMTSL1, E-Gene,
AsA; SVBP, E-Gene, AsA; TMEM187, E-Gene, AsA; AGTRAP, E-Gene, AsA; PTRF, E-Gene, AsA;
TRIM72, E-Gene, AsA; SCN4B, E-Gene, AsA; WDR73, E-Gene, AsA; TMEM80, E-Gene, AsA;
MTHFR, E-Gene, AsA; TOB2P1, E-Gene, AsA; SCAND2P, E-Gene, AsA; MAP3K19, E-Gene, AsA;
PHLDB3, E-Gene, AsA; DMRTA1, E-Gene, AsA; ZSCAN2, E-Gene, AsA; ORAI3, E-Gene, AsA;
DOK7, E-Gene, AsA; MSRA, E-Gene, AsA; EIF3KP1, E-Gene, AsA; B3GNTL1, E-Gene, AsA;
TOM1L2, E-Gene, AsA; RMI2, E-Gene, AsA; EIF1AD, E-Gene, AsA; PTPN2, E-Gene, AsA;
ATP2A2, E-Gene, AsA; GPR160, E-Gene, AsA; DPY19L1, E-Gene, AsA; TRAPPC3L, E-Gene,
AsA; RNF26, E-Gene, AsA; CCDC36, E-Gene, AsA; MAP3K11, E-Gene, AsA; PPP1R3B, E-Gene,
AsA; TNKS, E-Gene, AsA; IQCB1, E-Gene, AsA; MFSD4B, E-Gene, AsA; AHSA2, E-Gene, AsA;
ADCK5, E-Gene, AsA; HSPA6, E-Gene, AsA; HECTD4, E-Gene, AsA; DHCR7, E-Gene, AsA;
OVOL1, E-Gene, AsA; RASGRP1, E-Gene, AsA; FAM170B, E-Gene, AsA; HCFC1, E-Gene, AsA;
CLEC12A, E-Gene, AsA; LCE1D, E-Gene, AsA; NME6, E-Gene, AsA; KLF11, E-Gene, AsA;
LAMB2, E-Gene, AsA; DRC3, E-Gene, AsA; FRMD5, E-Gene, AsA; NHLH1, E-Gene, AsA;
LRRC34, E-Gene, AsA; PTGER4, E-Gene, AsA; NPTX1, E-Gene, AsA; TRIM8, E-Gene, AsA;
LINC00208, E-Gene, AsA; SDR16C5, E-Gene, AsA; NFXL1, E-Gene, AsA; ZNF212, E-Gene, AsA;
RNF150, E-Gene, AsA; GPR25, E-Gene, AsA; SPDYE5, E-Gene, AsA; UBE2E3, E-Gene, AsA;
MAP3K2, E-Gene, AsA; ZNF768, E-Gene, AsA; PYDC1, E-Gene, AsA; ITGAM, E-Gene, AsA;
HIC2, E-Gene, AsA; HINT1, E-Gene, AsA; CCDC8, E-Gene, AsA; RASSF6, E-Gene, AsA; PCSK9,
E-Gene, AsA; UPF3A, E-Gene, AsA; COMMD8, E-Gene, AsA; SPRY3, E-Gene, AsA; CTRB2, E-
Gene, AsA; CTRB1, E-Gene, AsA; TNIP2, E-Gene, AsA; IL12A, E-Gene, AsA; STAT3, E-Gene,
AsA; SLC20A2, E-Gene, AsA; FEN1, E-Gene, AsA; CCDC110, E-Gene, AsA; CX3CR1, E-Gene,
AsA; MOBP, E-Gene, AsA; RPSA, E-Gene, AsA; BEST1, E-Gene, AsA; KRT24, E-Gene, AsA;
TRPV3, E-Gene, AsA; PROSER3, E-Gene, AsA; VKORC1, E-Gene, AsA; ZNF646, E-Gene, AsA;
ZNF668, E-Gene, AsA; IRGQ, E-Gene, AsA; NOD2, E-Gene, AsA; DUSP18, E-Gene, AsA; STX3,
E-Gene, AsA; ATP23, E-Gene, AsA; STRCP1, E-Gene, AsA; PRKCB, E-Gene, AsA; XRRA1, E-
Gene, AsA; SYNPO2L, E-Gene, AsA; WBP1L, E-Gene, AsA; MYRFL, E-Gene, AsA; BRD7, E-
Gene, AsA; FUNDC2, E-Gene, AsA; CLEC1B, E-Gene, AsA; VSTM4, E-Gene, AsA; PCDH19, E-
Gene, AsA; NCF1C, E-Gene, AsA; GEM, E-Gene, AsA; DEFB1, E-Gene, AsA; HNF4G, E-Gene,
AsA; CTSB, E-Gene, AsA; PTTG1, E-Gene, AsA; SLC35A1, E-Gene, AsA; ACSL6, E-Gene, AsA;
TERT, E-Gene, AsA; ERAP2, E-Gene, AsA; ERAP1, E-Gene, AsA; PGGT1B, E-Gene, AsA;
NDUFAF2, E-Gene, AsA; ELOVL7, E-Gene, AsA; NDST3, E-Gene, AsA; GRM2, E-Gene, AsA;
TEX264, E-Gene, AsA; SHISA5, E-Gene, AsA; PLXNB1, E-Gene, AsA; FBXW12, E-Gene, AsA;
ZNF589, E-Gene, AsA; CAMP, E-Gene, AsA; CDC25A, E-Gene, AsA; BDH2, E-Gene, AsA;
ZNF691, E-Gene, AsA; CDCP1, E-Gene, AsA; ADAMTS9, E-Gene, AsA; ALB, E-Gene, AsA;
CTLA4, E-Gene, AsA; PRKCI, E-Gene, AsA; POGLUT1, E-Gene, AsA; C1orf106, E-Gene, AsA;
NIPAL1, E-Gene, AsA; DCAF16, E-Gene, AsA; COQ8A, E-Gene, AsA; H3F3A, E-Gene, AsA;
ARL5A, E-Gene, AsA; KIAA1841, E-Gene, AsA; PUS10, E-Gene, AsA; C1orf147, E-Gene, AsA;
KIF26B, E-Gene, AsA; LRRC52, E-Gene, AsA; VANGL2, E-Gene, AsA; NCSTN, E-Gene, AsA;
MYSM1, E-Gene, AsA; OMA1, E-Gene, AsA; WNT4, E-Gene, AsA; C1orf123, E-Gene, AsA;
SLC1A7, E-Gene, AsA; NEU3, E-Gene, AsA; SYCE2, E-Gene, AsA; RAVER1, E-Gene, AsA;
KLHL10, E-Gene, AsA; ZNF382, E-Gene, AsA; U2AF1L4, E-Gene, AsA; CCDC116, E-Gene, AsA;
YDJC, E-Gene, AsA; VPS28, E-Gene, AsA; UBQLN4, E-Gene, AsA; ANO10, E-Gene, AsA; VPS11,
E-Gene, AsA; C21orf2, E-Gene, AsA; ICOSLG, E-Gene, AsA; GAB3, E-Gene, AsA; RSPH1, E-
Gene, AsA; UBASH3A, E-Gene, AsA; NR2F6, E-Gene, AsA; ZFYVE28, E-Gene, AsA; HLCS, E-
Gene, AsA; SIM2, E-Gene, AsA; RAPGEF6, E-Gene, AsA; CDC42SE2, E-Gene, AsA; B4GALT3,
E-Gene, AsA; NIT1, E-Gene, AsA; F11R, E-Gene, AsA; ITLN2, E-Gene, AsA; ZSCAN12, E-Gene,
AsA; C12orf43, E-Gene, AsA; SPPL3, E-Gene, AsA; FCHO2, E-Gene, AsA; ADAMTSL3, E-Gene,
AsA; ADAMTS3, E-Gene, AsA; BATF, E-Gene, AsA; MCU, E-Gene, AsA; CLIC2, E-Gene, AsA;
RAB39B, E-Gene, AsA; VBP1, E-Gene, AsA; ELMO1, E-Gene, AsA; OXA1L, E-Gene, AsA;
PLCL2, E-Gene, AsA; MRPL39, E-Gene, AsA; PDLIM3, E-Gene, AsA; FAM167A, E-Gene, AsA;
TDH, E-Gene, AsA; JAZF1, E-Gene, AsA; TEX29, E-Gene, AsA; CAST, E-Gene, AsA; ANAPC1,
E-Gene, AsA; BANK1, E-Gene, AsA; RASGRP3, E-Gene, AsA; SPEF2, E-Gene, AsA; XRCC4, E-
Gene, AsA; ARL11, E-Gene, AsA; TMEM163, E-Gene, AsA; GLT1D1, E-Gene, AsA; CCDC122, E-
Gene, AsA; DPYSL4, E-Gene, AsA; FRMD4A, E-Gene, AsA; CDC123, E-Gene, AsA; FAM177A1,
E-Gene, AsA; DLG5, E-Gene, AsA; RAD9B, E-Gene, AsA; ANK3, E-Gene, AsA; PRSS53, E-Gene,
AsA; CD226, E-Gene, AsA; ITGB1, E-Gene, AsA; FADS1, E-Gene, AsA; GRIK4, E-Gene, AsA;
APIP, E-Gene, AsA; EIF4EBP2, E-Gene, AsA; DNAJB12, E-Gene, AsA; SEC16A, E-Gene, AsA;
INPP5E, E-Gene, AsA; STOM, E-Gene, AsA; NTRK2, E-Gene, AsA; SLC39A4, E-Gene, AsA;
RSPO2, E-Gene, AsA; RPL10, E-Gene, AsA; ZNF185, E-Gene, AsA; MFHAS1, E-Gene, AsA;
TMEM47, E-Gene, AsA; ASB15, E-Gene, AsA; POMZP3, E-Gene, AsA; IGFBP3, E-Gene, AsA;
ABRACL, E-Gene, AsA; RARS2, E-Gene, AsA; SCUBE3, E-Gene, AsA; HMGCLL1, E-Gene, AsA;
ABT1, E-Gene, AsA; MUT, E-Gene, AsA; PAM, E-Gene, AsA; PPIP5K2, E-Gene, AsA; GIN1, E-
Gene, AsA; CISD2, E-Gene, AsA; PYURF, E-Gene, AsA; MANF, E-Gene, AsA; AMT, E-Gene,
AsA; ADPRH, E-Gene, AsA; PLA1A, E-Gene, AsA; SLC25A38, E-Gene, AsA; CTDSP1, E-Gene,
AsA; UBXN4, E-Gene, AsA; GALNT2, E-Gene, AsA; RIT1, E-Gene, AsA; FCRL5, E-Gene, AsA;
SDHC, E-Gene, AsA; MGST3, E-Gene, AsA; ALDH9A1, E-Gene, AsA; IGSF3, E-Gene, AsA;
BCL10, E-Gene, AsA; SERBP1, E-Gene, AsA; AKT1, E-Gene, AsA; PFKL, E-Gene, AsA; RPTOR,
E-Gene, AsA; FN3KRP, E-Gene, AsA; TBCD, E-Gene, AsA; SCRN2, E-Gene, AsA; NPEPPS, E-
Gene, AsA; ARMC5, E-Gene, AsA; ITGAX, E-Gene, AsA; SCAMP2, E-Gene, AsA; ULK3, E-Gene,
AsA; TPM1, E-Gene, AsA; GPR65, E-Gene, AsA; VPS37B, E-Gene, AsA; SBNO1, E-Gene, AsA;
GLTP, E-Gene, AsA; SLC15A4, E-Gene, AsA; NDUFA9, E-Gene, AsA; KIAA1109, E-Gene, AsA;
NDST4, E-Gene, AsA; PPCDC, E-Gene, AsA; APH1B, E-Gene, AsA; COX17, E-Gene, AsA; NAB1,
E-Gene, AsA; ASNSD1, E-Gene, AsA; DNA2, E-Gene, AsA; RPS24, E-Gene, AsA; OIT3, E-Gene,
AsA; ZNF365, E-Gene, AsA; ASCC1, E-Gene, AsA; FAM149B1, E-Gene, AsA; MFSD13A, E-Gene,
AsA; ACTR1A, E-Gene, AsA; TMPRSS13, E-Gene, AsA; TMPRSS4, E-Gene, AsA; SLCO2B1, E-
Gene, AsA; RNF144B, E-Gene, AsA; PGBD1, E-Gene, AsA; ZSCAN9, E-Gene, AsA; TLR4, E-
Gene, AsA; IL1F10, E-Gene, AsA; IL36B, E-Gene, AsA; IL36RN, E-Gene, AsA; IL36A, E-Gene,
AsA; IL1RN, E-Gene, AsA; SKIL, E-Gene, AsA; BLK, E-Gene, AsA; ALPK3, E-Gene, AsA;
EDNRB, E-Gene, AsA; RCBTB1, E-Gene, AsA; SLC41A2, E-Gene, AsA; KCNMB4, E-Gene, AsA;
TEC, E-Gene, AsA; LTV1, E-Gene, AsA; SLC26A10, E-Gene, AsA; B4GALNT1, E-Gene, AsA;
PRR5L, E-Gene, AsA; ORC3, E-Gene, AsA; AKIRIN2, E-Gene, AsA; CCDC146, E-Gene, AsA;
TMEM258, E-Gene, AsA; FADS2, E-Gene, AsA; DAGLA, E-Gene, AsA; TIMM17A, E-Gene, AsA;
GRHL1, E-Gene, AsA; AP4B1, E-Gene, AsA; C1QTNF6, E-Gene, AsA; POSTN, E-Gene, AsA;
TRPC4, E-Gene, AsA; LPIN3, E-Gene, AsA; KIAA0907, E-Gene, AsA; BTBD3, E-Gene, AsA;
FIGNL1, E-Gene, AsA; RHPN2, E-Gene, AsA; NDFIP1, E-Gene, AsA; PSME3, E-Gene, AsA;
PDLIM4, E-Gene, AsA; C12orf65, E-Gene, AsA; LRP3, E-Gene, AsA; MPP1, E-Gene, AsA;
PLXNA3, E-Gene, AsA; DKC1, E-Gene, AsA; HIP1R, E-Gene, AsA; RSPH3, E-Gene, AsA; CDC16,
E-Gene, AsA; GALNT8, E-Gene, AsA; TNNI3, E-Gene, AsA; KRI1, E-Gene, AsA; VPS13C, E-
Gene, AsA; ORMDL1, E-Gene, AsA; OSGEPL1, E-Gene, AsA; GAD1, E-Gene, AsA; IRF5, E-Gene,
AsA; LIF, E-Gene, AsA; RAC2, E-Gene, AsA; MGAT3, E-Gene, AsA; SPINK2, E-Gene, AsA;
PMS2P3, E-Gene, AsA; EPS15L1, E-Gene, AsA; RAB3IP, E-Gene, AsA; WNK4, E-Gene, AsA;
COX6B1, E-Gene, AsA; IGFLR1, E-Gene, AsA; GFRA4, E-Gene, AsA; PSD4, E-Gene, AsA; IL37,
E-Gene, AsA; IL1B, E-Gene, AsA; SLC2A4RG, E-Gene, AsA; DOK4, E-Gene, AsA; MYRF, E-
Gene, AsA; DKFZP434K028, E-Gene, AsA; CDKN1A, E-Gene, AsA; TCP11, E-Gene, AsA; RPS10,
E-Gene, AsA; ZNF391, E-Gene, AsA; SNRPC, E-Gene, AsA; BTN1A1, E-Gene, AsA; LYPD3, E-
Gene, AsA; XG, E-Gene, AsA; SDC4, E-Gene, AsA; SLC12A5, E-Gene, AsA; PMS2P5, E-Gene,
AsA; MXD4, E-Gene, AsA; EBPL, E-Gene, AsA; HVCN1, E-Gene, AsA; IFT81, E-Gene, AsA;
CISD1, E-Gene, AsA; BICC1, E-Gene, AsA; CD244, E-Gene, AsA; COPA, E-Gene, AsA; ZRANB3,
E-Gene, AsA; CD80, E-Gene, AsA; POPDC2, E-Gene, AsA; ADCY7, E-Gene, AsA; UTP20, E-Gene,
AsA; TNFSF11, E-Gene, AsA; ENOX1, E-Gene, AsA; CNRIP1, E-Gene, AsA; CCDC92, E-Gene,
AsA; LDAH, E-Gene, AsA; SPP1, E-Gene, AsA; PKD2, E-Gene, AsA; MTR, E-Gene, AsA;
TMEM9, E-Gene, AsA; KIF21B, E-Gene, AsA; OLFML3, E-Gene, AsA; MFN2, E-Gene, AsA;
LAMTOR2, E-Gene, AsA; GRIN3B, E-Gene, AsA; DARS, E-Gene, AsA; USP34, E-Gene, AsA;
GLS, E-Gene, AsA; LANCL1, E-Gene, AsA; IL1A, E-Gene, AsA; ADAM23, E-Gene, AsA;
ARHGEF26, E-Gene, AsA; MAPKAPK3, E-Gene, AsA; HEMK1, E-Gene, AsA; FRMD4B, E-Gene,
AsA; CYB561D2, E-Gene, AsA; HYAL1, E-Gene, AsA; TIMMDC1, E-Gene, AsA; TCERG1, E-
Gene, AsA; TTC33, E-Gene, AsA; PPP2CA, E-Gene, AsA; SKP1, E-Gene, AsA; GNPDA1, E-Gene,
AsA; IL4, E-Gene, AsA; TARS, E-Gene, AsA; NPR3, E-Gene, AsA; PRSS16, E-Gene, AsA;
BTN2A1, E-Gene, AsA; FAM120B, E-Gene, AsA; CCR6, E-Gene, AsA; TRIM38, E-Gene, AsA;
COL9A1, E-Gene, AsA; FANCE, E-Gene, AsA; PPARD, E-Gene, AsA; HINT3, E-Gene, AsA;
SMIM8, E-Gene, AsA; DSE, E-Gene, AsA; KLRB1, E-Gene, AsA; RP11-22B23.1, E-Gene, AsA;
LPCAT3, E-Gene, AsA; OAS3, E-Gene, AsA; CDK2AP1, E-Gene, AsA; NAA25, E-Gene, AsA;
ALDH2, E-Gene, AsA; AKAP3, E-Gene, AsA; SH2B3, E-Gene, AsA; VPS29, E-Gene, AsA; GPN3,
E-Gene, AsA; TRPV4, E-Gene, AsA; MLEC, E-Gene, AsA; VWF, E-Gene, AsA; NCAPG, E-Gene,
AsA; UBE2D3, E-Gene, AsA; MANBA, E-Gene, AsA; NFKB1, E-Gene, AsA; SLAIN2, E-Gene,
AsA; HSD17B1P1, E-Gene, AsA; RAB5C, E-Gene, AsA; NUP88, E-Gene, AsA; RAI1, E-Gene,
AsA; KPNB1, E-Gene, AsA; PBLD, E-Gene, AsA; ZMIZ1, E-Gene, AsA; TFAM, E-Gene, AsA;
EDRF1, E-Gene, AsA; SUFU, E-Gene, AsA; MICU1, E-Gene, AsA; SPOCK2, E-Gene, AsA; VSIR,
E-Gene, AsA; KANK1, E-Gene, AsA; C5, E-Gene, AsA; LIMK1, E-Gene, AsA; NPTX2, E-Gene,
AsA; COBL, E-Gene, AsA; GRB10, E-Gene, AsA; VIPR2, E-Gene, AsA; PIK3CG, E-Gene, AsA;
CADM4, E-Gene, AsA; ETHE1, E-Gene, AsA; ETV2, E-Gene, AsA; UPK1A, E-Gene, AsA;
DNASE2, E-Gene, AsA; KLF1, E-Gene, AsA; GCDH, E-Gene, AsA; TMEM205, E-Gene, AsA;
CDC37, E-Gene, AsA; TYK2, E-Gene, AsA; ICAM5, E-Gene, AsA; MRPL4, E-Gene, AsA; PRKD2,
E-Gene, AsA; SLC1A5, E-Gene, AsA; KCTD9, E-Gene, AsA; MTMR9, E-Gene, AsA; ERI1, E-
Gene, AsA; ARMC1, E-Gene, AsA; EIF3E, E-Gene, AsA; JPH1, E-Gene, AsA; PLAT, E-Gene, AsA;
IKBKB, E-Gene, AsA; LACTB, E-Gene, AsA; KAT8, E-Gene, AsA; BCKDK, E-Gene, AsA; STX4,
E-Gene, AsA; SEC14L5, E-Gene, AsA; NAGPA, E-Gene, AsA; POLR2C, E-Gene, AsA; DGKH, E-
Gene, AsA; ARHGEF7, E-Gene, AsA; UBL4A, E-Gene, AsA; NAA10, E-Gene, AsA; MXRA5, E-
Gene, AsA; SIRPB1, E-Gene, AsA; CD40, E-Gene, AsA; PSMA6, E-Gene, AsA; SRP54, E-Gene,
AsA; PCNX1, E-Gene, AsA; MTMR3, E-Gene, AsA; ASCC2, E-Gene, AsA; TAB1, E-Gene, AsA;
SYNGR1, E-Gene, AsA; ZMAT5, E-Gene, AsA; RPL3, E-Gene, AsA; CABP7, E-Gene, AsA;
THOC5, E-Gene, AsA; SLC35E4, E-Gene, AsA; PES1, E-Gene, AsA; POLR2E, E-Gene, AsA;
SETD1A, E-Gene, AsA; HSD3B7, E-Gene, AsA; STX1B, E-Gene, AsA; MYO9B, E-Gene, AsA;
OCEL1, E-Gene, AsA; MAST3, E-Gene, AsA; JAK2, E-Gene, AsA; ZNF184, E-Gene, AsA;
HOOK2, E-Gene, AsA; PPP2R3C, E-Gene, AsA; ZFHX4, E-Gene, AsA; MYO15A, E-Gene, AsA;
DTX2, E-Gene, AsA; STRN4, E-Gene, AsA; ICAM1, E-Gene, AsA; RGS1, E-Gene, AsA; KCNH4,
E-Gene, AsA; BRAP, E-Gene, AsA; MAPKAPK5, E-Gene, AsA; COQ9, E-Gene, AsA; C3orf18, E-
Gene, AsA; DOCK3, E-Gene, AsA; ASAP3, E-Gene, AsA; SF3B2, E-Gene, AsA; SH3BP2, E-Gene,
AsA; MTMR2, E-Gene, AsA; LAT2, E-Gene, AsA; TMED2, E-Gene, AsA; CHERP, E-Gene, AsA;
MYNN, E-Gene, AsA; APOB, E-Gene, AsA; REST, E-Gene, AsA; NOA1, E-Gene, AsA; CYLD, E-
Gene, AsA; PLOD1, E-Gene, AsA; TNPO1, E-Gene, AsA; TRPM3, E-Gene, AsA; XPO1, E-Gene,
AsA; KCNK2, E-Gene, AsA; CCNT2, E-Gene, AsA; CACNA1S, E-Gene, AsA; TCF7, E-Gene, AsA;
MAGI3, E-Gene, AsA; KEAP1, E-Gene, AsA; PGM1, E-Gene, AsA; CARMIL1, E-Gene, AsA;
FDFT1, E-Gene, AsA; CDH17, E-Gene, AsA; VDAC3, E-Gene, AsA; LAMP3, E-Gene, AsA;
MCCC1, E-Gene, AsA; DYNC1I2, E-Gene, AsA; GPATCH1, E-Gene, AsA; MCM6, E-Gene, AsA;
TXK, E-Gene, AsA; SMARCE1, E-Gene, AsA; SCARB1, E-Gene, AsA; XRCC1, E-Gene, AsA;
P4HA2, E-Gene, AsA; UBE2D1, E-Gene, AsA; SREBF1, E-Gene, AsA; CYBRD1, E-Gene, AsA;
CPSF1, E-Gene, AsA; FAM3A, E-Gene, AsA; FAM50A, E-Gene, AsA; RPS6KA2, E-Gene, AsA;
POLB, E-Gene, AsA; RASSF1, E-Gene, AsA; PDZD4, E-Gene, AsA; MTFR1, E-Gene, AsA;
PDE4A, E-Gene, AsA; ASB1, E-Gene, AsA; WDR18, E-Gene, AsA; UHRF1BP1, E-Gene, AsA;
ZNF76, E-Gene, AsA; TAF11, E-Gene, AsA; PMS1, E-Gene, AsA; ABCA7, E-Gene, AsA; CNN2, E-
Gene, AsA; TNPO3, E-Gene, AsA; DGAT2, E-Gene, AsA; TRAF3IP2, E-Gene, AsA; GALC, E-
Gene, AsA; MCF2L2, E-Gene, AsA; USE1, E-Gene, AsA; PRSS8, E-Gene, AsA; BCAR1, E-Gene,
AsA; RFC2, E-Gene, AsA; UTS2, E-Gene, AsA; ERCC8, E-Gene, AsA; FAM120A, E-Gene, AsA;
HDAC9, E-Gene, AsA; MAP4, E-Gene, AsA; ANO2, E-Gene, AsA; POLR2B, E-Gene, AsA; CTNS,
E-Gene, AsA; RAI14, E-Gene, AsA; C6, E-Gene, AsA; CLEC16A, E-Gene, AsA; DEPDC1B, E-
Gene, AsA; PEX3, E-Gene, AsA; CENPQ, E-Gene, AsA; ARHGAP31, E-Gene, AsA; IBSP, E-Gene,
AsA; SLAMF7, E-Gene, AsA; CD44, E-Gene, AsA; RNASET2, E-Gene, AsA; DEF6, E-Gene, AsA;
AKAP11, E-Gene, AsA; RNH1, E-Gene, AsA; NLRP2, E-Gene, AsA; CPS1, E-Gene, AsA; RUNX3,
E-Gene, AsA; DNASE1L1, E-Gene, AsA; CLDN11, E-Gene, AsA; ZBTB32, E-Gene, AsA; ANOS1,
E-Gene, AsA; ABHD5, E-Gene, AsA; CLCN6, E-Gene, AsA; HFE, E-Gene, AsA; CD9, E-Gene,
AsA; DYRK4, E-Gene, AsA; REV3L, E-Gene, AsA; RPS20, E-Gene, AsA; PGLYRP1, E-Gene,
AsA; E2F2, E-Gene, AsA; CACNA2D2, E-Gene, AsA; NOS2, E-Gene, AsA; CX3CL1, E-Gene,
AsA; CIAPIN1, E-Gene, AsA; ZMYND10, E-Gene, AsA; ARHGAP33, E-Gene, AsA; HS3ST1, E-
Gene, AsA; ABCB5, P-Gene, AsA; ABTB2, P-Gene, AsA; ACAN, P-Gene, AsA; ACOXL, P-Gene,
AsA; ACOXL-AS1, P-Gene, AsA; ACTRT3, P-Gene, AsA; ADA, P-Gene, AsA; ADAM23, P-Gene,
AsA; ADAMTSL1, P-Gene, AsA; ADAMTSL3, P-Gene, AsA; ADCY7, P-Gene, AsA; ADGB, P-
Gene, AsA; ADO, P-Gene, AsA; AFF3, P-Gene, AsA; AKAP3, P-Gene, AsA; AKIRIN2, P-Gene,
AsA; ANK3, P-Gene, AsA; ANKRD17, P-Gene, AsA; ANO1, P-Gene, AsA; ANO10, P-Gene, AsA;
ANOS1, P-Gene, AsA; AP4B1, P-Gene, AsA; AP4B1-AS1, P-Gene, AsA; AP5B1, P-Gene, AsA;
APOB, P-Gene, AsA; ARFRP1, P-Gene, AsA; ARHGAP31, P-Gene, AsA; ARHGAP4, P-Gene, AsA;
ARHGAP45, P-Gene, AsA; ARSF, P-Gene, AsA; ASGR2, P-Gene, AsA; ATG5, P-Gene, AsA;
ATG7, P-Gene, AsA; ATOH7, P-Gene, AsA; AVPR2, P-Gene, AsA; B3GALT5, P-Gene, AsA;
B3GNTL1, P-Gene, AsA; B4GALNT1, P-Gene, AsA; BACH2, P-Gene, AsA; BANK1, P-Gene, AsA;
BATF, P-Gene, AsA; BCAR1, P-Gene, AsA; BCL9L, P-Gene, AsA; BCR, P-Gene, AsA; BLK, P-
Gene, AsA; BRCC3, P-Gene, AsA; BRD7, P-Gene, AsA; C12orf43, P-Gene, AsA; C19orf44, P-Gene,
AsA; C1orf52, P-Gene, AsA; C1orf53, P-Gene, AsA; C1QTNF5, P-Gene, AsA; CACNA1S, P-Gene,
AsA; CACNA2D2, P-Gene, AsA; CADM1, P-Gene, AsA; CARMIL1, P-Gene, AsA; CASC19, P-
Gene, AsA; CAST, P-Gene, AsA; CBLIF, P-Gene, AsA; CBLN2, P-Gene, AsA; CBX3P9, P-Gene,
AsA; CCDC112, P-Gene, AsA; CCDC8, P-Gene, AsA; CCL22, P-Gene, AsA; CCL7, P-Gene, AsA;
CCL8, P-Gene, AsA; CD226, P-Gene, AsA; CD247, P-Gene, AsA; CD6, P-Gene, AsA; CD99, P-
Gene, AsA; CDC123, P-Gene, AsA; CDH12, P-Gene, AsA; CDH17, P-Gene, AsA; CDH8, P-Gene,
AsA; CDKAL1, P-Gene, AsA; CEP128, P-Gene, AsA; CFAP97D2, P-Gene, AsA; CHCHD7, P-Gene,
AsA; CHERP, P-Gene, AsA; CHRDL2, P-Gene, AsA; CLCN6, P-Gene, AsA; CLEC12A-AS1, P-
Gene, AsA; CLEC16A, P-Gene, AsA; CMYA5, P-Gene, AsA; CNDP1, P-Gene, AsA; CNGB1, P-
Gene, AsA; CNIH3, P-Gene, AsA; CNRIP1, P-Gene, AsA; COBL, P-Gene, AsA; COL4A2, P-Gene,
AsA; COL9A1, P-Gene, AsA; CPNE5, P-Gene, AsA; CPS1, P-Gene, AsA; CPS1-IT1, P-Gene, AsA;
CR1, P-Gene, AsA; CRPPA, P-Gene, AsA; CSK, P-Gene, AsA; CSNK1G2P1, P-Gene, AsA;
CTDSP1, P-Gene, AsA; CTDSPL2, P-Gene, AsA; CTNNA3, P-Gene, AsA; CUX2, P-Gene, AsA;
CXCR5, P-Gene, AsA; DARS1, P-Gene, AsA; DARS1-AS1, P-Gene, AsA; DCLRE1B, P-Gene, AsA;
DGKH, P-Gene, AsA; DOCK3, P-Gene, AsA; DOCK5, P-Gene, AsA; DOK6, P-Gene, AsA; DPP6,
P-Gene, AsA; DPYD, P-Gene, AsA; DSE, P-Gene, AsA; DUSP10, P-Gene, AsA; EBF1, P-Gene,
AsA; EEA1, P-Gene, AsA; EFNA5, P-Gene, AsA; EIF3J-DT, P-Gene, AsA; ELMO1, P-Gene, AsA;
ELOVL7, P-Gene, AsA; ERBB3, P-Gene, AsA; ERC1, P-Gene, AsA; ETS1, P-Gene, AsA; EVC2, P-
Gene, AsA; FABP3P2, P-Gene, AsA; FADS1, P-Gene, AsA; FADS2, P-Gene, AsA; FAM120B, P-
Gene, AsA; FAM133GP, P-Gene, AsA; FAM167A, P-Gene, AsA; FAM76B, P-Gene, AsA; FBXL6,
P-Gene, AsA; FBXW2, P-Gene, AsA; FCGR2A, P-Gene, AsA; FCGR3B, P-Gene, AsA; FCRL5, P-
Gene, AsA; FDX2, P-Gene, AsA; FHIT, P-Gene, AsA; FKRP, P-Gene, AsA; FLI1, P-Gene, AsA;
FMN2, P-Gene, AsA; FRMD4A, P-Gene, AsA; FRMD4B, P-Gene, AsA; FTO, P-Gene, AsA;
FUCA2, P-Gene, AsA; FURIN, P-Gene, AsA; GABRB1, P-Gene, AsA; GALNT2, P-Gene, AsA;
GCDH, P-Gene, AsA; GFRA4, P-Gene, AsA; GJB6, P-Gene, AsA; GLTP, P-Gene, AsA; GNG2, P-
Gene, AsA; GPC5, P-Gene, AsA; GRIK4, P-Gene, AsA; GSN, P-Gene, AsA; H2AC6, P-Gene, AsA;
HDAC9, P-Gene, AsA; HERC6, P-Gene, AsA; HIP1, P-Gene, AsA; HIP1R, P-Gene, AsA; HISLA, P-
Gene, AsA; HLCS, P-Gene, AsA; HMGN2P2, P-Gene, AsA; HNF1A, P-Gene, AsA; HS3ST2, P-
Gene, AsA; HS6ST1, P-Gene, AsA; HSPB6, P-Gene, AsA; IGFBP3, P-Gene, AsA; IKBKE, P-Gene,
AsA; IL11RA, P-Gene, AsA; IL12A-AS1, P-Gene, AsA; IL18R1, P-Gene, AsA; IL1RAPL1, P-Gene,
AsA; IL21R, P-Gene, AsA; IL2RA, P-Gene, AsA; IL36B, P-Gene, AsA; IL4, P-Gene, AsA; INF2, P-
Gene, AsA; intron_variant, P-Gene, AsA; IPCEF1, P-Gene, AsA; IRAG1, P-Gene, AsA; IRAK1, P-
Gene, AsA; IRF5, P-Gene, AsA; IRGQ, P-Gene, AsA; ITGAX, P-Gene, AsA; ITGB1, P-Gene, AsA;
ITLN2, P-Gene, AsA; JAK2, P-Gene, AsA; JAKMIP3, P-Gene, AsA; JAZF1, P-Gene, AsA; KANK1,
P-Gene, AsA; KAZN, P-Gene, AsA; KCNK2, P-Gene, AsA; KCNMB2, P-Gene, AsA; KCNU1, P-
Gene, AsA; KEAP1, P-Gene, AsA; KIF26B, P-Gene, AsA; KLF1, P-Gene, AsA; KLF11, P-Gene,
AsA; KLK2, P-Gene, AsA; KLK3, P-Gene, AsA; KLRB1, P-Gene, AsA; KMO, P-Gene, AsA;
KRT223P, P-Gene, AsA; KRT8P21, P-Gene, AsA; LAMP3, P-Gene, AsA; LCE1B, P-Gene, AsA;
LCE1C, P-Gene, AsA; LCORL, P-Gene, AsA; LHX9, P-Gene, AsA; LILRA4, P-Gene, AsA;
LINC00687, P-Gene, AsA; LINC01100, P-Gene, AsA; LINC01509, P-Gene, AsA; LINC01845, P-
Gene, AsA; LINC01980, P-Gene, AsA; LINC02254, P-Gene, AsA; LINC02444, P-Gene, AsA;
LINC02470, P-Gene, AsA; LINC02580, P-Gene, AsA; LINC02640, P-Gene, AsA; LMNTD1, P-
Gene, AsA; LNC-LBCS, P-Gene, AsA; LOW, P-Gene, AsA; LPP, P-Gene, AsA; LRP1B, P-Gene,
AsA; LRRK2, P-Gene, AsA; LY9, P-Gene, AsA; MACIR, P-Gene, AsA; MACROD2, P-Gene, AsA;
MAGI2-AS3, P-Gene, AsA; MAML2, P-Gene, AsA; MANBA, P-Gene, AsA; MAP3K8, P-Gene,
AsA; MARCHF1, P-Gene, AsA; MAST3, P-Gene, AsA; MCU, P-Gene, AsA; MECP2, P-Gene, AsA;
MEIKIN, P-Gene, AsA; MFRP, P-Gene, AsA; MFSD4B, P-Gene, AsA; MIR2115, P-Gene, AsA;
MIR26B, P-Gene, AsA; MIR4686, P-Gene, AsA; MIR4696, P-Gene, AsA; MIR4804, P-Gene, AsA;
MIR718, P-Gene, AsA; MIR8072, P-Gene, AsA; MMP13, P-Gene, AsA; MOBP, P-Gene, AsA;
MODERATE, P-Gene, AsA; MODIFIER, P-Gene, AsA; MPP1, P-Gene, AsA; MRPS17P5, P-Gene,
AsA; MSR1, P-Gene, AsA; MTCO1P44, P-Gene, AsA; MTCP1, P-Gene, AsA; MTHFR, P-Gene,
AsA; MTMR3, P-Gene, AsA; MTND1P27, P-Gene, AsA; MTND2P19, P-Gene, AsA; MTR, P-Gene,
AsA; MXRA5, P-Gene, AsA; MYBPC1, P-Gene, AsA; MYNN, P-Gene, AsA; MYO9B, P-Gene,
AsA; MYRIP, P-Gene, AsA; NAALADL2, P-Gene, AsA; NAB1, P-Gene, AsA; NAGPA, P-Gene,
AsA; NBN, P-Gene, AsA; NCF2, P-Gene, AsA; NDST4, P-Gene, AsA; NDUFA3P6, P-Gene, AsA;
NEB, P-Gene, AsA; NEFHP1, P-Gene, AsA; NKAIN2, P-Gene, AsA; NLGN4X, P-Gene, AsA;
NLRP2, P-Gene, AsA; NMD_transcript_variant, P-Gene, AsA; NOD2, P-Gene, AsA; NOTCH1, P-
Gene, AsA; NPAS3, P-Gene, AsA; NR4A3, P-Gene, AsA; NRSN2, P-Gene, AsA; NTRK2, P-Gene,
AsA; OAS3, P-Gene, AsA; OPRM1, P-Gene, AsA; OR2W6P, P-Gene, AsA; ORC3, P-Gene, AsA;
OXA1L, P-Gene, AsA; OXA1L-DT, P-Gene, AsA; PA2G4, P-Gene, AsA; PAM, P-Gene, AsA;
PCDH11X, P-Gene, AsA; PCDH19, P-Gene, AsA; PCDH9, P-Gene, AsA; PCSK9, P-Gene, AsA;
PES1, P-Gene, AsA; PEX13, P-Gene, AsA; PEX7, P-Gene, AsA; PFKFB3, P-Gene, AsA; PGM1, P-
Gene, AsA; PHF2, P-Gene, AsA; PITPNC1, P-Gene, AsA; PKIG, P-Gene, AsA; PKP3, P-Gene, AsA;
PLA1A, P-Gene, AsA; PLCL2, P-Gene, AsA; PMS1, P-Gene, AsA; POLR2B, P-Gene, AsA;
POLR3A, P-Gene, AsA; POU2F1, P-Gene, AsA; PPM1H, P-Gene, AsA; PPP2R3B, P-Gene, AsA;
PRICKLE2, P-Gene, AsA; PRKCB, P-Gene, AsA; PRKN, P-Gene, AsA; PRORP, P-Gene, AsA;
PROSER3, P-Gene, AsA; PRR5L, P-Gene, AsA; PTPRC, P-Gene, AsA; PTPRH, P-Gene, AsA;
PTPRK, P-Gene, AsA; RAB44, P-Gene, AsA; RABGAP1, P-Gene, AsA; RAC2, P-Gene, AsA;
RASGRF2, P-Gene, AsA; RASGRP1, P-Gene, AsA; RASGRP3, P-Gene, AsA; REL, P-Gene, AsA;
REL-DT, P-Gene, AsA; REST, P-Gene, AsA; REV3L, P-Gene, AsA; RFC2, P-Gene, AsA; RFLNA,
P-Gene, AsA; RGS21, P-Gene, AsA; RHPN2, P-Gene, AsA; RMI2, P-Gene, AsA; RN7SKP258, P-
Gene, AsA; RN7SL441P, P-Gene, AsA; RN7SL561P, P-Gene, AsA; RNF150, P-Gene, AsA;
RNF217, P-Gene, AsA; RNF26, P-Gene, AsA; RNU6-234P, P-Gene, AsA; RNU6-474P, P-Gene,
AsA; RNU6-565P, P-Gene, AsA; RNU6-919P, P-Gene, AsA; RNU6-927P, P-Gene, AsA; RPL13AP7,
P-Gene, AsA; RPL27AP3, P-Gene, AsA; RPL35AP13, P-Gene, AsA; RPL36AP25, P-Gene, AsA;
RPL5P21, P-Gene, AsA; RPS23P9, P-Gene, AsA; RPS6P25, P-Gene, AsA; RPTOR, P-Gene, AsA;
RSBN1, P-Gene, AsA; RTEL1, P-Gene, AsA; RUNX3, P-Gene, AsA; RYR3, P-Gene, AsA; SBNO1,
P-Gene, AsA; SBNO1-AS1, P-Gene, AsA; SCN7A, P-Gene, AsA; SCUBE1, P-Gene, AsA; SCUBE3,
P-Gene, AsA; SDC4, P-Gene, AsA; SEC14L5, P-Gene, AsA; SEC61A2, P-Gene, AsA; SEPTIN7P1,
P-Gene, AsA; SIGIRR, P-Gene, AsA; SIGLEC6, P-Gene, AsA; SIRPG, P-Gene, AsA; SIRPG-AS1,
P-Gene, AsA; SKIL, P-Gene, AsA; SLAIN1, P-Gene, AsA; SLC11A1, P-Gene, AsA; SLC12A5, P-
Gene, AsA; SLC14A2, P-Gene, AsA; SLC15A4, P-Gene, AsA; SLC1A7, P-Gene, AsA; SLC20A2, P-
Gene, AsA; SLC25A6, P-Gene, AsA; SLC45A2, P-Gene, AsA; SLC52A2, P-Gene, AsA; SLC6A20,
P-Gene, AsA; SNED1, P-Gene, AsA; SNRPCP2, P-Gene, AsA; SOX13, P-Gene, AsA; SOX5, P-
Gene, AsA; SPINK8, P-Gene, AsA; SPRED2, P-Gene, AsA; SRRM5, P-Gene, AsA; SSPOP, P-Gene,
AsA; SSR2, P-Gene, AsA; STAT3, P-Gene, AsA; STAT4, P-Gene, AsA; STOM, P-Gene, AsA;
STUM, P-Gene, AsA; SUFU, P-Gene, AsA; SYMBOL, P-Gene, AsA; SYNGR1, P-Gene, AsA;
SYT1, P-Gene, AsA; TAGAP, P-Gene, AsA; TCEA3, P-Gene, AsA; TCF7, P-Gene, AsA; TEC, P-
Gene, AsA; TERT, P-Gene, AsA; TFAM, P-Gene, AsA; TH, P-Gene, AsA; THEMIS, P-Gene, AsA;
THSD7A, P-Gene, AsA; TIMM17A, P-Gene, AsA; TLK1, P-Gene, AsA; TLR5, P-Gene, AsA;
TMEM163, P-Gene, AsA; TMEM229B, P-Gene, AsA; TMEM232, P-Gene, AsA; TMEM249, P-
Gene, AsA; TMPRSS13, P-Gene, AsA; TMPRSS4, P-Gene, AsA; TNFRSF13B, P-Gene, AsA;
TNFSF15, P-Gene, AsA; TNIP1, P-Gene, AsA; TNIP2, P-Gene, AsA; TNKS, P-Gene, AsA; TNPO1,
P-Gene, AsA; TOX, P-Gene, AsA; TPM1, P-Gene, AsA; TPM1-AS, P-Gene, AsA; TRAK1, P-Gene,
AsA; TRIB3, P-Gene, AsA; TRIM44, P-Gene, AsA; TRPC4, P-Gene, AsA; TRPM3, P-Gene, AsA;
TRPV1, P-Gene, AsA; TUBB4BP1, P-Gene, AsA; TXNRD1, P-Gene, AsA; UBASH3A, P-Gene,
AsA; UBE2E3, P-Gene, AsA; UBE2L3, P-Gene, AsA; UGT3A1, P-Gene, AsA; UPP2, P-Gene, AsA;
UVRAG, P-Gene, AsA; VANGL2, P-Gene, AsA; VDAC1P8, P-Gene, AsA; VIPR2, P-Gene, AsA;
VSTM4, P-Gene, AsA; VWF, P-Gene, AsA; WARS1P1, P-Gene, AsA; WDFY4, P-Gene, AsA;
WWOX, P-Gene, AsA; XKR6, P-Gene, AsA; Y_RNA, P-Gene, AsA; ZBTB7C, P-Gene, AsA;
ZBTB7C-AS1, P-Gene, AsA; ZGLP1, P-Gene, AsA; ZGPAT, P-Gene, AsA; ZMIZ1, P-Gene, AsA;
ZMIZ1-AS1, P-Gene, AsA; ZMYND8, P-Gene, AsA; ZNF184, P-Gene, AsA; ZNF185, P-Gene, AsA;
ZNF212, P-Gene, AsA; ZNF423, P-Gene, AsA; ZNF576, P-Gene, AsA; ZNF875, P-Gene, AsA;
ABT1, T-Gene, AsA; ABTB2, T-Gene, AsA; AC004231.2, T-Gene, AsA; AC011363.1, T-Gene,
AsA; AC017083.1, T-Gene, AsA; AC017083.2, T-Gene, AsA; AC020743.2, T-Gene, AsA;
AC023024.1, T-Gene, AsA; AC025594.1, T-Gene, AsA; AC026801.2, T-Gene, AsA; AC079779.4, T-
Gene, AsA; AC092159.3, T-Gene, AsA; AC092327.1, T-Gene, AsA; AC093390.1, T-Gene, AsA;
AC114808.2, T-Gene, AsA; AC124014.1, T-Gene, AsA; AC130454.2, T-Gene, AsA; ACP1, T-Gene,
AsA; ADO, T-Gene, AsA; AF131215.3, T-Gene, AsA; AKAP11, T-Gene, AsA; AL136987.1, T-
Gene, AsA; AL138696.1, T-Gene, AsA; AL138810.1, T-Gene, AsA; AL611946.1, T-Gene, AsA;
ALDH1A3, T-Gene, AsA; ALPL, T-Gene, AsA; ANAPC16, T-Gene, AsA; ANKRD27, T-Gene,
AsA; APIP, T-Gene, AsA; ARHGAP17, T-Gene, AsA; ARMC5, T-Gene, AsA; ASB15, T-Gene,
AsA; ASB7, T-Gene, AsA; ASCC1, T-Gene, AsA; ATF4, T-Gene, AsA; ATP6V0A2, T-Gene, AsA;
AUH, T-Gene, AsA; BAIAP2L1, T-Gene, AsA; BCKDK, T-Gene, AsA; BLK, T-Gene, AsA; BRIX1,
T-Gene, AsA; BTG1, T-Gene, AsA; BTN2A2, T-Gene, AsA; BTN3A2, T-Gene, AsA; C10orf105, T-
Gene, AsA; C10orf54, T-Gene, AsA; C10orf68, T-Gene, AsA; C12orf65, T-Gene, AsA; C13orf30, T-
Gene, AsA; C16orf74, T-Gene, AsA; C19orf40, T-Gene, AsA; C1QA, T-Gene, AsA; C1QB, T-Gene,
AsA; C1QC, T-Gene, AsA; C1QTNF2, T-Gene, AsA; C5orf54, T-Gene, AsA; C7orf72, T-Gene, AsA;
C8orf12, T-Gene, AsA; CACNG3, T-Gene, AsA; CALU, T-Gene, AsA; CAPRIN1, T-Gene, AsA;
CAT, T-Gene, AsA; CBX7, T-Gene, AsA; CCDC122, T-Gene, AsA; CCDC7, T-Gene, AsA; CCNJL,
T-Gene, AsA; CCR7, T-Gene, AsA; CD44, T-Gene, AsA; CDC16, T-Gene, AsA; CDC6, T-Gene,
AsA; CDC73, T-Gene, AsA; CDH23, T-Gene, AsA; CDK2AP1, T-Gene, AsA; CDKL3, T-Gene,
AsA; CEBPG, T-Gene, AsA; CEP89, T-Gene, AsA; CERS3, T-Gene, AsA; CHST3, T-Gene, AsA;
CHSY1, T-Gene, AsA; CLYBL, T-Gene, AsA; CNRIP1, T-Gene, AsA; COBL, T-Gene, AsA;
COTL1, T-Gene, AsA; COX4I1, T-Gene, AsA; COX4NB, T-Gene, AsA; COX6A2, T-Gene, AsA;
CPSF6, T-Gene, AsA; CTC348L5.1, T-Gene, AsA; CTC493P15.3, T-Gene, AsA; CTC529G1.1, T-
Gene, AsA; CTD3076017.1, T-Gene, AsA; DCUN1D2AS2, T-Gene, AsA; DDIT4, T-Gene, AsA;
DDX46, T-Gene, AsA; DDX55, T-Gene, AsA; DIRAS2, T-Gene, AsA; DLEU1, T-Gene, AsA;
DLEU2, T-Gene, AsA; DLEU7, T-Gene, AsA; DLEU7AS1, T-Gene, AsA; DNAH10, T-Gene, AsA;
DNAJB12, T-Gene, AsA; DNAJC15, T-Gene, AsA; DOCK9, T-Gene, AsA; DOCK9AS1, T-Gene,
AsA; DYNC2LI1, T-Gene, AsA; EBPL, T-Gene, AsA; EGFR, T-Gene, AsA; EGR2, T-Gene, AsA;
EIF1, T-Gene, AsA; ENOX1, T-Gene, AsA; EPHA8, T-Gene, AsA; EPHB2, T-Gene, AsA; EPSTI1,
T-Gene, AsA; FAM124A, T-Gene, AsA; FAM150B, T-Gene, AsA; FAM167A, T-Gene, AsA;
FAM70B, T-Gene, AsA; FAM71F2, T-Gene, AsA; FBXO31, T-Gene, AsA; FBXO48, T-Gene, AsA;
FBXO9, T-Gene, AsA; FIGNL1, T-Gene, AsA; FLJ30679, T-Gene, AsA; FLJ41484, T-Gene, AsA;
FLJ44054, T-Gene, AsA; FLNC, T-Gene, AsA; FOXC2, T-Gene, AsA; FOXF1, T-Gene, AsA; FUS,
T-Gene, AsA; GAS6, T-Gene, AsA; GAS6AS1, T-Gene, AsA; GGA2, T-Gene, AsA; GINS2, T-Gene,
AsA; GJD3, T-Gene, AsA; GPATCH1, T-Gene, AsA; GPR18, T-Gene, AsA; GPR183, T-Gene, AsA;
GSTA1, T-Gene, AsA; GUSBP2, T-Gene, AsA; HCAR1, T-Gene, AsA; HDAC7, T-Gene, AsA;
HIST1H1E, T-Gene, AsA; HIST1H2AD, T-Gene, AsA; HIST1H2AG, T-Gene, AsA; HIST1H2BJ, T-
Gene, AsA; HIST1H2BK, T-Gene, AsA; HIST1H2BO, T-Gene, AsA; HIST1H3E, T-Gene, AsA;
HIST1H3G, T-Gene, AsA; HIST1H4C, T-Gene, AsA; HIST1H4G, T-Gene, AsA; HIST1H4H, T-
Gene, AsA; HIST1H4I, T-Gene, AsA; HMGN1, T-Gene, AsA; HMGN4, T-Gene, AsA; HSD3B7, T-
Gene, AsA; HSPG2, T-Gene, AsA; ICK, T-Gene, AsA; IGFBP4, T-Gene, AsA; IKZF1, T-Gene, AsA;
IRF5, T-Gene, AsA; IRF8, T-Gene, AsA; ITGAD, T-Gene, AsA; ITGAX, T-Gene, AsA; ITGB1, T-
Gene, AsA; ITLN2, T-Gene, AsA; KAT8, T-Gene, AsA; KCNRG, T-Gene, AsA; KCTD15, T-Gene,
AsA; KIAA0182, T-Gene, AsA; KIF5B, T-Gene, AsA; KRT14, T-Gene, AsA; KRT15, T-Gene, AsA;
KRT20, T-Gene, AsA; KRT222, T-Gene, AsA; KRT24, T-Gene, AsA; KRT25, T-Gene, AsA;
KRT26, T-Gene, AsA; KRT27, T-Gene, AsA; KRT28, T-Gene, AsA; KRT42P, T-Gene, AsA; KRT9,
T-Gene, AsA; LACC1, T-Gene, AsA; LACTBL1, T-Gene, AsA; LDLRAD3, T-Gene, AsA;
LINC00240, T-Gene, AsA; LINS, T-Gene, AsA; LMO2, T-Gene, AsA; LOC100129316, T-Gene,
AsA; LOC100129726, T-Gene, AsA; LOC100270746, T-Gene, AsA; LOC100506394, T-Gene, AsA;
LOC146513, T-Gene, AsA; LOC256021, T-Gene, AsA; LOC400550, T-Gene, AsA; LOC730101, T-
Gene, AsA; LOC732275, T-Gene, AsA; LRP3, T-Gene, AsA; LRRK1, T-Gene, AsA; LUZP1, T-
Gene, AsA; MGAT3, T-Gene, AsA; MIR1278, T-Gene, AsA; MIR12892, T-Gene, AsA; MIR146A,
T-Gene, AsA; MIR3142, T-Gene, AsA; MIR548AN, T-Gene, AsA; MIR623, T-Gene, AsA; MIR762,
T-Gene, AsA; MSL1, T-Gene, AsA; NCRNA00223, T-Gene, AsA; NDUFAB1, T-Gene, AsA; NFIL3,
T-Gene, AsA; NPTX2, T-Gene, AsA; NRBF2, T-Gene, AsA; NRP1, T-Gene, AsA; NUDT19, T-
Gene, AsA; OGFOD2, T-Gene, AsA; OPN1SW, T-Gene, AsA; OXER1, T-Gene, AsA; PA2G4, T-
Gene, AsA; PARD3, T-Gene, AsA; PCCAAS1, T-Gene, AsA; PDCD5, T-Gene, AsA; PDHX, T-
Gene, AsA; PHF15, T-Gene, AsA; PLEK, T-Gene, AsA; PPP2CA, T-Gene, AsA; PRKCB, T-Gene,
AsA; PRSS16, T-Gene, AsA; PRSS36, T-Gene, AsA; PRSS53, T-Gene, AsA; PRSS8, T-Gene, AsA;
PSAP, T-Gene, AsA; PTTG1, T-Gene, AsA; PYCARD, T-Gene, AsA; PYDC1, T-Gene, AsA; RAD1,
T-Gene, AsA; RAI14, T-Gene, AsA; RARA, T-Gene, AsA; RASA3, T-Gene, AsA; REEP3, T-Gene,
AsA; RGS1, T-Gene, AsA; RGS13, T-Gene, AsA; RGS2, T-Gene, AsA; RHPN2, T-Gene, AsA;
RILPL1, T-Gene, AsA; RILPL2, T-Gene, AsA; RNASEH2B, T-Gene, AsA; RNF40, T-Gene, AsA;
RNU716P, T-Gene, AsA; RNU738P, T-Gene, AsA; RNU86, T-Gene, AsA; ROR2, T-Gene, AsA;
RP11148O21.3, T-Gene, AsA; RP11148O21.4, T-Gene, AsA; RP11214F16.2, T-Gene, AsA;
RP11215P8.3, T-Gene, AsA; RP11286H14.8, T-Gene, AsA; RP11309L24.2, T-Gene, AsA;
RP11332K15.1, T-Gene, AsA; RP11342D11.2, T-Gene, AsA; RP11541P9.3, T-Gene, AsA;
RP11546D6.2, T-Gene, AsA; RP1169E9.1, T-Gene, AsA; RP1190B22.2, T-Gene, AsA; RP127K12.4,
T-Gene, AsA; RP168D18.3, T-Gene, AsA; RP4607I7.1, T-Gene, AsA; RP4683L5.1, T-Gene, AsA;
RP4724E13.2, T-Gene, AsA; RPL3, T-Gene, AsA; RPL6, T-Gene, AsA; RPS19BP1, T-Gene, AsA;
SBNO1, T-Gene, AsA; SELS, T-Gene, AsA; SETD1A, T-Gene, AsA; SETD8, T-Gene, AsA; SKP1,
T-Gene, AsA; SLC1A2, T-Gene, AsA; SLC29A3, T-Gene, AsA; SLU7, T-Gene, AsA; SMARCE1, T-
Gene, AsA; SMCR7L, T-Gene, AsA; SNORA36.1, T-Gene, AsA; SNORA68.1, T-Gene, AsA;
SNORD83A, T-Gene, AsA; snoU109.3, T-Gene, AsA; snoU13.218, T-Gene, AsA; snoU13.248, T-
Gene, AsA; SNRNP35, T-Gene, AsA; SNRPA1, T-Gene, AsA; SNRPC, T-Gene, AsA; SPOCK2, T-
Gene, AsA; SPRYD7, T-Gene, AsA; ST13P4, T-Gene, AsA; STX1B, T-Gene, AsA; STX4, T-Gene,
AsA; SVIL, T-Gene, AsA; SYK, T-Gene, AsA; SYNGR1, T-Gene, AsA; TAB1, T-Gene, AsA;
TBC1D10B, T-Gene, AsA; TCF7, T-Gene, AsA; TDRD12, T-Gene, AsA; THADA, T-Gene, AsA;
TM9SF2, T-Gene, AsA; TMED2, T-Gene, AsA; TMEM130, T-Gene, AsA; TMEM18, T-Gene, AsA;
TMEM99, T-Gene, AsA; TNFSF11, T-Gene, AsA; TNPO3, T-Gene, AsA; TNS4, T-Gene, AsA;
TOP2A, T-Gene, AsA; TPI1P2, T-Gene, AsA; TRIM72, T-Gene, AsA; TRIM8, T-Gene, AsA;
TRNAI2, T-Gene, AsA; TSPAN33, T-Gene, AsA; TTC23L, T-Gene, AsA; U1.108, T-Gene, AsA;
U2.33, T-Gene, AsA; U6.1198, T-Gene, AsA; U6.1250, T-Gene, AsA; U6.26, T-Gene, AsA; U6.414,
T-Gene, AsA; U6.992, T-Gene, AsA; U7.110, T-Gene, AsA; U7.115, T-Gene, AsA; UBAC2, T-Gene,
AsA; UBAC2AS1, T-Gene, AsA; UPF3A, T-Gene, AsA; UVRAG, T-Gene, AsA; VDAC1, T-Gene,
AsA; VPS37B, T-Gene, AsA; WDR88, T-Gene, AsA; WDR92, T-Gene, AsA; WIPF2, T-Gene, AsA;
WNT4, T-Gene, AsA; XKR6, T-Gene, AsA; ZDHHC7, T-Gene, AsA; ZFP36L2, T-Gene, AsA;
ZNF322, T-Gene, AsA; ZNF646, T-Gene, AsA; ZNF668, T-Gene, AsA; ZNF720, T-Gene, AsA;
ZNF828, T-Gene, AsA; ZPBP, T-Gene, AsA; CDH17, C-Genes, EA; IDNK, C-Genes, EA; OTUD3,
C-Genes, EA; QARS, C-Genes, EA; CR2, C-Genes, EA; FCGR2A, C-Genes, EA; AKAP3, C-Genes,
EA; NT5E, C-Genes, EA; TNFAIP3, C-Genes, EA; TYK2, C-Genes, EA; IFIH1, C-Genes, EA; CR1,
C-Genes, EA; EML2, C-Genes, EA; AGBL2, C-Genes, EA; AC007193.8, E-Gene, EA; AC007193.9,
E-Gene, EA; AC022201.5, E-Gene, EA; AC079753.5, E-Gene, EA; AC091132.1, E-Gene, EA;
AC109333.10, E-Gene, EA; AC124789.1, E-Gene, EA; ACSS2, E-Gene, EA; ADAMTS9-AS2, E-
Gene, EA; AHSA2, E-Gene, EA; AJ006998.2, E-Gene, EA; ALOX15, E-Gene, EA; ANKRD44, E-
Gene, EA; AP3B2, E-Gene, EA; APLP1, E-Gene, EA; ARHGAP27, E-Gene, EA; ARL17B, E-Gene,
EA; ARL3, E-Gene, EA; ARRB2, E-Gene, EA; AS3MT, E-Gene, EA; B3GNT2, E-Gene, EA;
BCAR1, E-Gene, EA; BLK, E-Gene, EA; BTN3A2, E-Gene, EA; C2orf42, E-Gene, EA; CAMTA2,
E-Gene, EA; CARM1, E-Gene, EA; CASP10, E-Gene, EA; CASP8, E-Gene, EA; CAST, E-Gene,
EA; CD38, E-Gene, EA; CHAC1, E-Gene, EA; CHCHD2, E-Gene, EA; CHP1, E-Gene, EA;
CIAPIN1, E-Gene, EA; CNKSR1, E-Gene, EA; COQ9, E-Gene, EA; CRHR1, E-Gene, EA; CRHR1-
IT1, E-Gene, EA; CTB-39G8.3, E-Gene, EA; CTC-506B8.1, E-Gene, EA; CTD-2260A17.3, E-Gene,
EA; CTD-2561B21.4, E-Gene, EA; CTRB1, E-Gene, EA; CTRB2, E-Gene, EA; CXCL16, E-Gene,
EA; CYP17A1-AS1, E-Gene, EA; DDX27, E-Gene, EA; DND1P1, E-Gene, EA; DOK4, E-Gene, EA;
EDEM2, E-Gene, EA; EIF6, E-Gene, EA; ENO3, E-Gene, EA; ETV7, E-Gene, EA; EXTL1, E-Gene,
EA; FAM136A, E-Gene, EA; FAM215B, E-Gene, EA; FAM69A, E-Gene, EA; FAM83C, E-Gene,
EA; FDFT1, E-Gene, EA; FMNL1, E-Gene, EA; GP1BA, E-Gene, EA; GPX4, E-Gene, EA;
GRPEL1, E-Gene, EA; GTF2H5, E-Gene, EA; GTF2IRD2, E-Gene, EA; HLCS, E-Gene, EA;
IL12RB1, E-Gene, EA; IL1RL1, E-Gene, EA; IL37, E-Gene, EA; IMPG2, E-Gene, EA; INO80, E-
Gene, EA; KANSL1, E-Gene, EA; KANSL1-AS1, E-Gene, EA; KCNB1, E-Gene, EA; KIAA1841, E-
Gene, EA; KIF1C, E-Gene, EA; KNSTRN, E-Gene, EA; LRRC37A, E-Gene, EA; LRRC37A17P, E-
Gene, EA; LRRC37A2, E-Gene, EA; LRRC37A4P, E-Gene, EA; MAP1LC3A, E-Gene, EA; MAPT,
E-Gene, EA; MAPT-AS1, E-Gene, EA; MIR1307, E-Gene, EA; MMP16, E-Gene, EA; MMP24, E-
Gene, EA; MRPL45, E-Gene, EA; MTFR1L, E-Gene, EA; MYH7B, E-Gene, EA; NCF2, E-Gene,
EA; NDUFAF1, E-Gene, EA; NEK7, E-Gene, EA; NIF3L1, E-Gene, EA; NMNAT1P1, E-Gene, EA;
NPTX1, E-Gene, EA; NRD1, E-Gene, EA; NSF, E-Gene, EA; NSFP1, E-Gene, EA; NT5C2, E-Gene,
EA; NT5C3B, E-Gene, EA; NT5E, E-Gene, EA; NUPR1L, E-Gene, EA; NUSAP1, E-Gene, EA;
OIP5, E-Gene, EA; OIP5-AS1, E-Gene, EA; OTUD3, E-Gene, EA; PAFAH2, E-Gene, EA; PAPOLG,
E-Gene, EA; PCYOX1, E-Gene, EA; PDIK1L, E-Gene, EA; PEBP1, E-Gene, EA; PELP1, E-Gene,
EA; PINX1, E-Gene, EA; PLCL1, E-Gene, EA; PLEK, E-Gene, EA; PLEKHM1, E-Gene, EA;
PMS2P5, E-Gene, EA; POLR2C, E-Gene, EA; PPIL3, E-Gene, EA; PPP5C, E-Gene, EA; PROCR, E-
Gene, EA; PRSS55, E-Gene, EA; PSPHP1, E-Gene, EA; PTPN7, E-Gene, EA; PXDN, E-Gene, EA;
RAB3B, E-Gene, EA; RNF167, E-Gene, EA; RNU6-312P, E-Gene, EA; RP1-317E23.3, E-Gene, EA;
RP1-317E23.7, E-Gene, EA; RP1-50J22.4, E-Gene, EA; RP11-135D11.2, E-Gene, EA; RP11-
155O18.6, E-Gene, EA; RP11-209K10.2, E-Gene, EA; RP11-20G6.2, E-Gene, EA; RP11-259G18.1,
E-Gene, EA; RP11-259G18.2, E-Gene, EA; RP11-259G18.3, E-Gene, EA; RP11-293E1.1, E-Gene,
EA; RP11-30P6.6, E-Gene, EA; RP11-350J20.5, E-Gene, EA; RP11-367J11.3, E-Gene, EA; RP11-
383F6.1, E-Gene, EA; RP11-386I23.1, E-Gene, EA; RP11-448G4.2, E-Gene, EA; RP11-669E14.4, E-
Gene, EA; RP11-669E14.6, E-Gene, EA; RP11-707O23.5, E-Gene, EA; RP11-724N1.1, E-Gene, EA;
RP11-753C18.8, E-Gene, EA; RP11-798G7.5, E-Gene, EA; RP11-798G7.8, E-Gene, EA; RP1L1, E-
Gene, EA; RP4-614O4.11, E-Gene, EA; RP4-614O4.12, E-Gene, EA; RP4-657D16.3, E-Gene, EA;
RP4-725G10.4, E-Gene, EA; RPAP1, E-Gene, EA; RPS26P8, E-Gene, EA; RPTOR, E-Gene, EA;
rs4644492, E-Gene, EA; RSBN1, E-Gene, EA; RTF1, E-Gene, EA; SDHC, E-Gene, EA; SENP7, E-
Gene, EA; SERAC1, E-Gene, EA; SF3B1, E-Gene, EA; SFXN2, E-Gene, EA; SIM2, E-Gene, EA;
SLC30A2, E-Gene, EA; SLC9A4, E-Gene, EA; SMARCA4, E-Gene, EA; SNHG5, E-Gene, EA;
SPAG7, E-Gene, EA; SPPL2C, E-Gene, EA; STAG3L1, E-Gene, EA; STAG3L2, E-Gene, EA;
SUDS3, E-Gene, EA; SYN2, E-Gene, EA; SYNJ2, E-Gene, EA; TADA2B, E-Gene, EA; TAX1BP3,
E-Gene, EA; TIMP4, E-Gene, EA; TMEM180, E-Gene, EA; TMEM57, E-Gene, EA; TRIM24, E-
Gene, EA; TRIM38, E-Gene, EA; TRIM63, E-Gene, EA; TRPC4AP, E-Gene, EA; UBASH3A, E-
Gene, EA; ULBP3, E-Gene, EA; UQCC1, E-Gene, EA; USP31, E-Gene, EA; VSIG10, E-Gene, EA;
WNT3, E-Gene, EA; YIPF2, E-Gene, EA; ZC3HAV1, E-Gene, EA; ZC3HAV1L, E-Gene, EA;
ZMYND15, E-Gene, EA; ZNF713, E-Gene, EA; FGFRL1, E-Gene, EA; KIF18A, E-Gene, EA;
SLC39A7, E-Gene, EA; ZDHHC16, E-Gene, EA; ASB16, E-Gene, EA; ABCB1, P-Gene, EA;
ABCC4, P-Gene, EA; ACTRT2, P-Gene, EA; ADAD1, P-Gene, EA; ADAM15, P-Gene, EA;
ADCYAP1R1, P-Gene, EA; ADORA3, P-Gene, EA; ADPRH, P-Gene, EA; AFF3, P-Gene, EA;
AGAP1, P-Gene, EA; AGBL2, P-Gene, EA; AICDA, P-Gene, EA; AIPL1, P-Gene, EA; AIRE, P-
Gene, EA; AK055570, P-Gene, EA; AK056252, P-Gene, EA; AK057451, P-Gene, EA; AK093525, P-
Gene, EA; AK094607, P-Gene, EA; AK094674, P-Gene, EA; AK094715, P-Gene, EA; AK097902, P-
Gene, EA; AK123834, P-Gene, EA; AK125001, P-Gene, EA; AKAP3, P-Gene, EA; AL832909, P-
Gene, EA; ALS2CR12, P-Gene, EA; AMPH, P-Gene, EA; ANG, P-Gene, EA; ANK3, P-Gene, EA;
ANKRD55, P-Gene, EA; ANP32B, P-Gene, EA; ANXA2P3, P-Gene, EA; ARHGAP31, P-Gene, EA;
ARHGAP44, P-Gene, EA; ARHGEF2, P-Gene, EA; ARID3A, P-Gene, EA; ARID5B, P-Gene, EA;
ARL1, P-Gene, EA; ARL4C, P-Gene, EA; ARPC2, P-Gene, EA; ARSI, P-Gene, EA; ASB3, P-Gene,
EA; ASCL2, P-Gene, EA; ATG16L2, P-Gene, EA; ATG5, P-Gene, EA; ATXN1, P-Gene, EA;
AX746871, P-Gene, EA; AX747401, P-Gene, EA; AX747415, P-Gene, EA; BACH2, P-Gene, EA;
BC009730, P-Gene, EA; BC032030, P-Gene, EA; BC032899, P-Gene, EA; BC034612, P-Gene, EA;
BC034940, P-Gene, EA; BC035094, P-Gene, EA; BC037927, P-Gene, EA; BC038767, P-Gene, EA;
BC038792, P-Gene, EA; BC039437, P-Gene, EA; BC041470, P-Gene, EA; BC046497, P-Gene, EA;
BC048118, P-Gene, EA; BC065754, P-Gene, EA; BC073918, P-Gene, EA; BC105019, P-Gene, EA;
BCAT1, P-Gene, EA; BCL2L11, P-Gene, EA; BCL6, P-Gene, EA; BEND3, P-Gene, EA; BNIP2, P-
Gene, EA; C12orf28, P-Gene, EA; C12orf49, P-Gene, EA; C15orf59, P-Gene, EA; C17orf51, P-Gene,
EA; C18orf1, P-Gene, EA; C1orf226, P-Gene, EA; C1orf55, P-Gene, EA; C5orf30, P-Gene, EA;
C6orf211, P-Gene, EA; C9orf103, P-Gene, EA; CA10, P-Gene, EA; CACNA1C, P-Gene, EA;
CACNA2D2, P-Gene, EA; CACNA2D3, P-Gene, EA; CADM1, P-Gene, EA; CAMK2D, P-Gene,
EA; CAMKK2, P-Gene, EA; CASP1, P-Gene, EA; CASR, P-Gene, EA; CBFA2T2, P-Gene, EA;
CCDC113, P-Gene, EA; CCDC155, P-Gene, EA; CCL17, P-Gene, EA; CCL22, P-Gene, EA; CCND1,
P-Gene, EA; CCR6, P-Gene, EA; CCR9, P-Gene, EA; CD2, P-Gene, EA; CD276, P-Gene, EA; CD5,
P-Gene, EA; CD6, P-Gene, EA; CD69, P-Gene, EA; CD80, P-Gene, EA; CD86, P-Gene, EA; CDH17,
P-Gene, EA; CDKAL1, P-Gene, EA; CDKL3, P-Gene, EA; CELF2, P-Gene, EA; CENPW, P-Gene,
EA; CEP57L1, P-Gene, EA; CHIT1, P-Gene, EA; CHRFAM7A, P-Gene, EA; CIAPIN1, P-Gene, EA;
CLEC16A, P-Gene, EA; CLEC4E, P-Gene, EA; CLECL1, P-Gene, EA; CLNK, P-Gene, EA;
CNRIP1, P-Gene, EA; COBL, P-Gene, EA; COL6A3, P-Gene, EA; COQ10B, P-Gene, EA; COQ9, P-
Gene, EA; COX7B2, P-Gene, EA; CPPED1, P-Gene, EA; CPSF3L, P-Gene, EA; CR1, P-Gene, EA;
CR2, P-Gene, EA; CSMD1, P-Gene, EA; CTAGE10, P-Gene, EA; CTLA4, P-Gene, EA; CTSH, P-
Gene, EA; CTXN3, P-Gene, EA; CUX1, P-Gene, EA; CX3CL1, P-Gene, EA; CXCR1, P-Gene, EA;
CXCR5, P-Gene, EA; DDX6, P-Gene, EA; DENND1A, P-Gene, EA; DEPTOR, P-Gene, EA; DGKH,
P-Gene, EA; DHCR7, P-Gene, EA; DKKL1, P-Gene, EA; DLC1, P-Gene, EA; DLEU1, P-Gene, EA;
DLG1, P-Gene, EA; DLGAP4, P-Gene, EA; DNAH6, P-Gene, EA; DNMT3A, P-Gene, EA;
DNMT3L, P-Gene, EA; DOCK8, P-Gene, EA; DOK4, P-Gene, EA; DOT1L, P-Gene, EA;
DQ586005, P-Gene, EA; DYTN, P-Gene, EA; EEPD1, P-Gene, EA; EFNA1, P-Gene, EA; ELK3, P-
Gene, EA; ELL, P-Gene, EA; EML2, P-Gene, EA; ENG, P-Gene, EA; ENTHD1, P-Gene, EA;
EPB41L4A, P-Gene, EA; ERBB2, P-Gene, EA; ETV6, P-Gene, EA; ETV7, P-Gene, EA; EVI5, P-
Gene, EA; EXOC2, P-Gene, EA; EYS, P-Gene, EA; FAM120B, P-Gene, EA; FAM181B, P-Gene,
EA; FAM184B, P-Gene, EA; FAM19A2, P-Gene, EA; FAM201A, P-Gene, EA; FAM27L, P-Gene,
EA; FAM69A, P-Gene, EA; FAM76B, P-Gene, EA; FARP1, P-Gene, EA; FARP2, P-Gene, EA;
FARS2, P-Gene, EA; FBN3, P-Gene, EA; FBRS, P-Gene, EA; FCGR2A, P-Gene, EA; FCHO2, P-
Gene, EA; FCRL1, P-Gene, EA; FKBP8, P-Gene, EA; FLJ33360, P-Gene, EA; FLJ45079, P-Gene,
EA; FLT1, P-Gene, EA; FOXB1, P-Gene, EA; GABRA4, P-Gene, EA; GABRB1, P-Gene, EA;
GALNT1, P-Gene, EA; GFI1, P-Gene, EA; GIMAP7, P-Gene, EA; GIMAP8, P-Gene, EA; GJA5, P-
Gene, EA; GLIS3, P-Gene, EA; GLT25D2, P-Gene, EA; GLUL, P-Gene, EA; GPR39, P-Gene, EA;
GRAP2, P-Gene, EA; GRB10, P-Gene, EA; GRM4, P-Gene, EA; GRM7, P-Gene, EA; GSDMB, P-
Gene, EA; HIF3A, P-Gene, EA; HLF, P-Gene, EA; HMP19, P-Gene, EA; HOXC9, P-Gene, EA;
HS3ST4, P-Gene, EA; HS6ST1, P-Gene, EA; HTA, P-Gene, EA; HTR5A, P-Gene, EA; ICA1, P-
Gene, EA; ICOS, P-Gene, EA; IFI16, P-Gene, EA; IFIH1, P-Gene, EA; IFNG, P-Gene, EA; IKZF2, P-
Gene, EA; IKZF3, P-Gene, EA; IKZF4, P-Gene, EA; IL10, P-Gene, EA; IL10RA, P-Gene, EA;
IL12A, P-Gene, EA; IL12RB1, P-Gene, EA; IL17RD, P-Gene, EA; IL18BP, P-Gene, EA; IL1R1, P-
Gene, EA; IL1R2, P-Gene, EA; IL2, P-Gene, EA; IL21, P-Gene, EA; IL28A, P-Gene, EA; IL29, P-
Gene, EA; IL2RA, P-Gene, EA; IL7R, P-Gene, EA; ILF3, P-Gene, EA; INO80D, P-Gene, EA;
INSIG2, P-Gene, EA; INSL4, P-Gene, EA; IPMK, P-Gene, EA; IPO5, P-Gene, EA; IPO8, P-Gene,
EA; IRF2BPL, P-Gene, EA; IRF7, P-Gene, EA; IRF8, P-Gene, EA; ITGAM, P-Gene, EA; ITPR2, P-
Gene, EA; JARID2, P-Gene, EA; JMJD1C, P-Gene, EA; KANK1, P-Gene, EA; KCNG1, P-Gene, EA;
KCNK2, P-Gene, EA; KCTD19, P-Gene, EA; KDM4B, P-Gene, EA; KIF1A, P-Gene, EA; KIF5A, P-
Gene, EA; KIRREL2, P-Gene, EA; KLF12, P-Gene, EA; KLF4, P-Gene, EA; KLF9, P-Gene, EA;
LARGE, P-Gene, EA; LBH, P-Gene, EA; LCT, P-Gene, EA; LINC00476, P-Gene, EA; LINC00477,
P-Gene, EA; LOC100128977, P-Gene, EA; LOC100129620, P-Gene, EA; LOC100130298, P-Gene,
EA; LOC100130451, P-Gene, EA; LOC100130476, P-Gene, EA; LOC100130581, P-Gene, EA;
LOC100131234, P-Gene, EA; LOC100288428, P-Gene, EA; LOC100506178, P-Gene, EA;
LOC150622, P-Gene, EA; LOC152225, P-Gene, EA; LOC283143, P-Gene, EA; LOC283867, P-
Gene, EA; LOC284751, P-Gene, EA; LOC285627, P-Gene, EA; LOC339290, P-Gene, EA;
LOC401134, P-Gene, EA; LOC541472, P-Gene, EA; LPHN3, P-Gene, EA; LPP-AS2, P-Gene, EA;
LRRC25, P-Gene, EA; LRRC34, P-Gene, EA; LTA4H, P-Gene, EA; LYN, P-Gene, EA; LYST, P-
Gene, EA; MAD1L1, P-Gene, EA; MAP1LC3B2, P-Gene, EA; MAP4K4, P-Gene, EA; MAST3, P-
Gene, EA; MGAT5, P-Gene, EA; MGAT5B, P-Gene, EA; MIR187, P-Gene, EA; MIR196A2, P-
Gene, EA; MIR320B1, P-Gene, EA; MIR3659, P-Gene, EA; MIR4686, P-Gene, EA; MIR4792, P-
Gene, EA; MIR548AN, P-Gene, EA; MKKS, P-Gene, EA; MORF4L1, P-Gene, EA; MPHOSPH9, P-
Gene, EA; MSRB2, P-Gene, EA; MTHFR, P-Gene, EA; MTRR, P-Gene, EA; MYCBP2, P-Gene, EA;
MYCNOS, P-Gene, EA; MYNN, P-Gene, EA; NALCN, P-Gene, EA; NANS, P-Gene, EA; NARS2,
P-Gene, EA; NEDD4L, P-Gene, EA; NEK6, P-Gene, EA; NEUROD6, P-Gene, EA; NFATC2, P-
Gene, EA; NFKBIZ, P-Gene, EA; NKX2, P-Gene, EA; NKX3, P-Gene, EA; NMNAT2, P-Gene, EA;
NOS2, P-Gene, EA; NOTCH2, P-Gene, EA; NOVA1, P-Gene, EA; NPAS2, P-Gene, EA; NRD1, P-
Gene, EA; NRG3, P-Gene, EA; NRP2, P-Gene, EA; NTM, P-Gene, EA; ODZ2, P-Gene, EA; ODZ4,
P-Gene, EA; OLIG3, P-Gene, EA; OR6S1, P-Gene, EA; ORMDL3, P-Gene, EA; P2RY6, P-Gene,
EA; PAPOLG, P-Gene, EA; PCDH15, P-Gene, EA; PCDH7, P-Gene, EA; PCIF1, P-Gene, EA;
PCK1, P-Gene, EA; PCNXL2, P-Gene, EA; PCSK9, P-Gene, EA; PDE3B, P-Gene, EA; PDS5A, P-
Gene, EA; PELI1, P-Gene, EA; PIGT, P-Gene, EA; PLA1A, P-Gene, EA; PLA2G2A, P-Gene, EA;
PLA2G2E, P-Gene, EA; PLCB1, P-Gene, EA; PLCB2, P-Gene, EA; PLCL2, P-Gene, EA; PLEK, P-
Gene, EA; PLEKHA6, P-Gene, EA; PLLP, P-Gene, EA; PLTP, P-Gene, EA; PLXDC2, P-Gene, EA;
POGLUT1, P-Gene, EA; POLR2C, P-Gene, EA; POP4, P-Gene, EA; PPIP5K2, P-Gene, EA; PPP5C,
P-Gene, EA; PRDM2, P-Gene, EA; PRR14, P-Gene, EA; PSMB1, P-Gene, EA; PTF1A, P-Gene, EA;
PTPN1, P-Gene, EA; PTPN2, P-Gene, EA; PTPRC, P-Gene, EA; PTPRD, P-Gene, EA; PTPRM, P-
Gene, EA; PUM2, P-Gene, EA; PVT1, P-Gene, EA; PXK, P-Gene, EA; QARS, P-Gene, EA;
RAD23B, P-Gene, EA; RAPGEF5, P-Gene, EA; RARB, P-Gene, EA; RBFOX1, P-Gene, EA;
RBMS3, P-Gene, EA; RHOB, P-Gene, EA; RIMBP2, P-Gene, EA; RLN2, P-Gene, EA; RNLS, P-
Gene, EA; RORA, P-Gene, EA; RPAP2, P-Gene, EA; SATB1, P-Gene, EA; SDC1, P-Gene, EA;
SF3B1, P-Gene, EA; SH2D1B, P-Gene, EA; SH2D4A, P-Gene, EA; SH3BP4, P-Gene, EA;
SH3PXD2B, P-Gene, EA; SHROOM3, P-Gene, EA; SIK2, P-Gene, EA; SKAP2, P-Gene, EA; SKI,
P-Gene, EA; SLAMF7, P-Gene, EA; SLC14A2, P-Gene, EA; SLC22A11, P-Gene, EA; SLC25A28,
P-Gene, EA; SLC2A13, P-Gene, EA; SLC2A9, P-Gene, EA; SLCO3A1, P-Gene, EA; SNX14, P-
Gene, EA; SNX19, P-Gene, EA; SORCS2, P-Gene, EA; SPEF2, P-Gene, EA; SPRED2, P-Gene, EA;
SPRYD7, P-Gene, EA; SRCA, P-Gene, EA; SRRT, P-Gene, EA; SRSF6, P-Gene, EA; SSBP4, P-
Gene, EA; ST8SIA4, P-Gene, EA; STARD10, P-Gene, EA; SYN3, P-Gene, EA; TASP1, P-Gene, EA;
TBX3, P-Gene, EA; TCEA3, P-Gene, EA; TCF7, P-Gene, EA; TCOF1, P-Gene, EA; TEC, P-Gene,
EA; THADA, P-Gene, EA; THSD4, P-Gene, EA; THSD7A, P-Gene, EA; TIMMDC1, P-Gene, EA;
TMEM18, P-Gene, EA; TMEM39A, P-Gene, EA; TNC, P-Gene, EA; TNFAIP3, P-Gene, EA;
TNFSF13B, P-Gene, EA; TNFSF8, P-Gene, EA; TNIK, P-Gene, EA; TNPO1, P-Gene, EA; TRAF3,
P-Gene, EA; TRMT11, P-Gene, EA; TRPC3, P-Gene, EA; TRPM3, P-Gene, EA; TTC28, P-Gene,
EA; TTC34, P-Gene, EA; TTYH1, P-Gene, EA; TWIST2, P-Gene, EA; TYK2, P-Gene, EA; UBE2K,
P-Gene, EA; UBXN2B, P-Gene, EA; USP14, P-Gene, EA; UTP23, P-Gene, EA; VAC14, P-Gene,
EA; VAV2, P-Gene, EA; VDAC1, P-Gene, EA; VENTXP7, P-Gene, EA; WDR64, P-Gene, EA;
WFDC2, P-Gene, EA; WSCD1, P-Gene, EA; YPEL5, P-Gene, EA; ZAN, P-Gene, EA; ZBP1, P-
Gene, EA; ZC3H4, P-Gene, EA; ZNF648, P-Gene, EA; ZPBP2, P-Gene, EA; A2ML1-AS2, T-Genes,
EA; ABCG4, T-Genes, EA; ABHD6, T-Genes, EA; ABP1, T-Genes, EA; AC010973.1, T-Genes, EA;
AC020743.2, T-Genes, EA; AC023102.1, T-Genes, EA; AC068570.1, T-Genes, EA; AC069304.1, T-
Genes, EA; AC084198.1, T-Genes, EA; AC092327.1, T-Genes, EA; AC109326.1, T-Genes, EA;
AC124014.1, T-Genes, EA; ACOX2, T-Genes, EA; ACSS2, T-Genes, EA; ADGRL3, T-Genes, EA;
ADNP, T-Genes, EA; AFMID, T-Genes, EA; AGAP3, T-Genes, EA; AL163636.2, T-Genes, EA;
ALG2, T-Genes, EA; ANAPC15, T-Genes, EA; ANG, T-Genes, EA; ANGEL1, T-Genes, EA;
ANKRD44, T-Genes, EA; ANXA6, T-Genes, EA; AP002954.3, T-Genes, EA; APEX1, T-Genes, EA;
APOBEC4, T-Genes, EA; ARL4D, T-Genes, EA; ARPC5, T-Genes, EA; ARRDC2, T-Genes, EA;
ASAP3, T-Genes, EA; ATF6, T-Genes, EA; ATP5H, T-Genes, EA; ATP5L, T-Genes, EA; BCAS4,
T-Genes, EA; BCL9L, T-Genes, EA; BEND3, T-Genes, EA; BHMG1, T-Genes, EA; BNIP2, T-
Genes, EA; BOLL, T-Genes, EA; BRAC1, T-Genes, EA; BX248409.1, T-Genes, EA; C14orf166B, T-
Genes, EA; C15orf62, T-Genes, EA; C16orf74, T-Genes, EA; C17ord108, T-Genes, EA; C1orf105, T-
Genes, EA; C1orf9, T-Genes, EA; C7orf72, T-Genes, EA; C9ord30-TMEFF1, T-Genes, EA;
CACNB1, T-Genes, EA; CASKIN2, T-Genes, EA; CCDC56, T-Genes, EA; CCDC61, T-Genes, EA;
CCDC69, T-Genes, EA; CCDC84, T-Genes, EA; CCNB1IP1, T-Genes, EA; CCNJL, T-Genes, EA;
CCR10, T-Genes, EA; CD3E, T-Genes, EA; CD40, T-Genes, EA; CD74, T-Genes, EA; CDK5RAP3,
T-Genes, EA; CDKL3, T-Genes, EA; CEBPB, T-Genes, EA; CENPL, T-Genes, EA; CEP192, T-
Genes, EA; CEP250, T-Genes, EA; CEP97, T-Genes, EA; CHAC1, T-Genes, EA; CHD7, T-Genes,
EA; CHURC1, T-Genes, EA; CHURC1-FNTB, T-Genes, EA; CIAPIN1, T-Genes, EA; CLEC4D, T-
Genes, EA; CLEC4E, T-Genes, EA; CLEC6A, T-Genes, EA; CLVS1, T-Genes, EA; COQ10B, T-
Genes, EA; COQ9, T-Genes, EA; COTL1, T-Genes, EA; COX4I1, T-Genes, EA; COX4NB, T-Genes,
EA; CTC-529G1.1, T-Genes, EA; CTD-2292M16.7, T-Genes, EA; CTSA, T-Genes, EA; CXCR5, T-
Genes, EA; DARS2, T-Genes, EA; DBIL5P, T-Genes, EA; DCTN4, T-Genes, EA; DDX46, T-Genes,
EA; DDX6, T-Genes, EA; DENND6A, T-Genes, EA; DHX8, T-Genes, EA; DISP2, T-Genes, EA;
DKFZP564C196, T-Genes, EA; DLD, T-Genes, EA; DLL4, T-Genes, EA; DNM3, T-Genes, EA;
DOK4, T-Genes, EA; DPAGT1, T-Genes, EA; DUSP12, T-Genes, EA; DYNLRB1, T-Genes, EA;
EDDM3A, T-Genes, EA; EDEM2, T-Genes, EA; EIF5A, T-Genes, EA; EIF6, T-Genes, EA; ELL, T-
Genes, EA; ELMO2, T-Genes, EA; ENSG00000202434, T-Genes, EA; ENSG00000225956, T-Genes,
EA; ENSG00000226439, T-Genes, EA; ENSG00000230686, T-Genes, EA; ENSG00000243295, T-
Genes, EA; ENSG00000254469, T-Genes, EA; ENSG00000260302, T-Genes, EA;
ENSG00000267480, T-Genes, EA; ENSG00000268810, T-Genes, EA; ENSG00000271420, T-Genes,
EA; ENSG00000272182, T-Genes, EA; ENSG00000273493, T-Genes, EA; ENSG00000278153, T-
Genes, EA; ERCC1, T-Genes, EA; EXD1, T-Genes, EA; EXTL1, T-Genes, EA; FAM107A, T-Genes,
EA; FAM163B, T-Genes, EA; FAM192A, T-Genes, EA; FAM55C, T-Genes, EA; FAM83C, T-
Genes, EA; FAM86C1, T-Genes, EA; FASLG, T-Genes, EA; FBXO31, T-Genes, EA; FCER1G, T-
Genes, EA; FCGR2A, T-Genes, EA; FCHO1, T-Genes, EA; FCRLA, T-Genes, EA; FCRLB, T-
Genes, EA; FIGNL1, T-Genes, EA; FKBP8, T-Genes, EA; FLJ30679, T-Genes, EA; FLNB, T-Genes,
EA; FLNB-AS1, T-Genes, EA; FOXB1, T-Genes, EA; FOXC2, T-Genes, EA; FOXF1, T-Genes, EA;
FOXR1, T-Genes, EA; G3BP1, T-Genes, EA; GAS5, T-Genes, EA; GBX1, T-Genes, EA;
GC01M023400, T-Genes, EA; GC02M128357, T-Genes, EA; GC02P191043, T-Genes, EA;
GC02P191061, T-Genes, EA; GC03M006543, T-Genes, EA; GC03M007443, T-Genes, EA;
GC04P061479, T-Genes, EA; GC05P151028, T-Genes, EA; GC05P160465, T-Genes, EA;
GC05P160475, T-Genes, EA; GC06M107116, T-Genes, EA; GC09P133952, T-Genes, EA;
GC09P133983, T-Genes, EA; GC10P006063, T-Genes, EA; GC11P072002, T-Genes, EA;
GC11P111651, T-Genes, EA; GC18M015662, T-Genes, EA; GC18M016019, T-Genes, EA;
GC18P012884, T-Genes, EA; GC20P046113, T-Genes, EA; GDF5, T-Genes, EA; GDI2, T-Genes,
EA; GEMIN4, T-Genes, EA; GGT7, T-Genes, EA; GIMAP1, T-Genes, EA; GIMAP4, T-Genes, EA;
GIMAP5, T-Genes, EA; GIMAP6, T-Genes, EA; GIMAP7, T-Genes, EA; GIMAP8, T-Genes, EA;
GINS2, T-Genes, EA; GLS, T-Genes, EA; GLT25D2, T-Genes, EA; GPRC5C, T-Genes, EA; GRM7,
T-Genes, EA; GRM7-AS1, T-Genes, EA; GSDMA, T-Genes, EA; GSDMB, T-Genes, EA; GSS, T-
Genes, EA; GSTZ1, T-Genes, EA; GTF3C3, T-Genes, EA; GTFA2A, T-Genes, EA; H2AFX, T-
Genes, EA; H3F3B, T-Genes, EA; HINFP, T-Genes, EA; HMGB3P1, T-Genes, EA; HMGN1P30, T-
Genes, EA; HSPA6, T-Genes, EA; HSPD1, T-Genes, EA; HSPE1, T-Genes, EA; HTD2, T-Genes,
EA; IFI30, T-Genes, EA; IFI35, T-Genes, EA; IKZF1, T-Genes, EA; IKZF3, T-Genes, EA; IL12RB1,
T-Genes, EA; IL15RA, T-Genes, EA; IL18BP, T-Genes, EA; IL2RA, T-Genes, EA; INAFM1, T-
Genes, EA; INO80, T-Genes, EA; IRF2BPL, T-Genes, EA; IRF8, T-Genes, EA; IRGM, T-Genes, EA;
ISM1, T-Genes, EA; ISM1-AS1, T-Genes, EA; ITCH, T-Genes, EA; JAK3, T-Genes, EA; KCNH2,
T-Genes, EA; KCTD2, T-Genes, EA; KCTD6, T-Genes, EA; KIAA0182, T-Genes, EA; KIAA1737,
T-Genes, EA; KLHL20, T-Genes, EA; KLHL33, T-Genes, EA; LAMC1, T-Genes, EA; LAMC2, T-
Genes, EA; LAMTOR1, T-Genes, EA; LOC100130476, T-Genes, EA; LOC100130581, T-Genes, EA;
LOC100506023, T-Genes, EA; LOC100506046, T-Genes, EA; LOC105372143, T-Genes, EA;
LOC105373610, T-Genes, EA; LOC105373805, T-Genes, EA; LOC105377106, T-Genes, EA;
LOC146513, T-Genes, EA; LOC152225, T-Genes, EA; LOC284751, T-Genes, EA; LOC400550, T-
Genes, EA; LOC732275, T-Genes, EA; LRRC25, T-Genes, EA; LRRC3C, T-Genes, EA; LRTOMT,
T-Genes, EA; MAP1LC3A, T-Genes, EA; MAST3, T-Genes, EA; MED1, T-Genes, EA; MED24, T-
Genes, EA; MEOX1, T-Genes, EA; METTL3, T-Genes, EA; MFAP5, T-Genes, EA; MIR-1289-2, T-
Genes, EA; MIR1289-1, T-Genes, EA; MIR1302-5, T-Genes, EA; MIR199A2, T-Genes, EA; MIR21,
T-Genes, EA; MIR2117, T-Genes, EA; MIR3142, T-Genes, EA; MIR3142HG, T-Genes, EA;
MIR4492, T-Genes, EA; MIR4736, T-Genes, EA; MIR499, T-Genes, EA; MIR499B, T-Genes, EA;
MIR645, T-Genes, EA; MMP24, T-Genes, EA; MNT, T-Genes, EA; MT1E, T-Genes, EA; MTHFSD,
T-Genes, EA; MYC, T-Genes, EA; MYH7B, T-Genes, EA; MYOC, T-Genes, EA; NARF, T-Genes,
EA; NARR, T-Genes, EA; NBR1, T-Genes, EA; NBR2, T-Genes, EA; NCF2, T-Genes, EA; NCOA5,
T-Genes, EA; NCOA6, T-Genes, EA; NDUFS2, T-Genes, EA; NEDD4L, T-Genes, EA; NEURL2, T-
Genes, EA; NFKBIZ, T-Genes, EA; NGB, T-Genes, EA; NLRX1, T-Genes, EA; NMAT2, T-Genes,
EA; NPM1P46, T-Genes, EA; NR4A3, T-Genes, EA; NUFIP2, T-Genes, EA; OIP5, T-Genes, EA;
OR11H4, T-Genes, EA; OR6S1, T-Genes, EA; ORMDL3, T-Genes, EA; OSGEP, T-Genes, EA;
PAFAH2, T-Genes, EA; PAQR7, T-Genes, EA; PARP2, T-Genes, EA; PCH1, T-Genes, EA; PCIF1,
T-Genes, EA; PDE12, T-Genes, EA; PDGFRB, T-Genes, EA; PFKFB3, T-Genes, EA; PGAP3, T-
Genes, EA; PHF15, T-Genes, EA; PHLDB1, T-Genes, EA; PIGC, T-Genes, EA; PIGU, T-Genes, EA;
PIR32263, T-Genes, EA; PIR51928, T-Genes, EA; PITPNC1, T-Genes, EA; PLCL1, T-Genes, EA;
PLEK, T-Genes, EA; PLEKHH3, T-Genes, EA; PLTP, T-Genes, EA; PNP, T-Genes, EA; POLR2C,
T-Genes, EA; POMT2, T-Genes, EA; PPP2CA, T-Genes, EA; PPP2R1B, T-Genes, EA; PPP5C, T-
Genes, EA; PRDX6, T-Genes, EA; PROCR, T-Genes, EA; PTGES3L, T-Genes, EA; PTGES3L-
AARSD1, T-Genes, EA; PTPN1, T-Genes, EA; PTPN2, T-Genes, EA; PTTG1, T-Genes, EA; PVT1,
T-Genes, EA; PXK, T-Genes, EA; QPCTL, T-Genes, EA; RAB34, T-Genes, EA; RAD51, T-Genes,
EA; RARRES2, T-Genes, EA; RBM17, T-Genes, EA; RBM22, T-Genes, EA; RGL1, T-Genes, EA;
RNASE1, T-Genes, EA; RNASE10, T-Genes, EA; RNASE11, T-Genes, EA; RNASE4, T-Genes, EA;
RNASE6, T-Genes, EA; RND2, T-Genes, EA; RNF114, T-Genes, EA; RNF121, T-Genes, EA;
RNU2-4P, T-Genes, EA; RNU6-1029P, T-Genes, EA; RP1-15D23.2, T-Genes, EA; RP1-6818.3, T-
Genes, EA; RP11-110I1.12, T-Genes, EA; RP11-203M5.7, T-Genes, EA; RP11-215P8.3, T-Genes,
EA; RP11-219E7.1, T-Genes, EA; RP11-33I11.2, T-Genes, EA; RP11-346K17.4, T-Genes, EA;
RP11-488C13.6, T-Genes, EA; RP11-4914.3, T-Genes, EA; RP11-541P9.3, T-Genes, EA; RP11-
770J1.4, T-Genes, EA; RP11-787D18.1, T-Genes, EA; RP11-84C10.4, T-Genes, EA; RP11-91I20.4,
T-Genes, EA; RP11-998D10.1, T-Genes, EA; RP11-998D10.8, T-Genes, EA; RP3-477O4.14, T-
Genes, EA; RP4-530I15.9, T-Genes, EA; RP4-584D14.6, T-Genes, EA; RP5-1070G24.2, T-Genes,
EA; RPAP1, T-Genes, EA; RPL13P2, T-Genes, EA; RPL19, T-Genes, EA; RPL23AP64, T-Genes,
EA; RPL24, T-Genes, EA; RPL27, T-Genes, EA; RPL32P23, T-Genes, EA; RPL7P1, T-Genes, EA;
RPP14, T-Genes, EA; RPS14, T-Genes, EA; RPS25, T-Genes, EA; RTF1, T-Genes, EA; RUNDC1,
T-Genes, EA; SAMD15, T-Genes, EA; SCARNA11.1, T-Genes, EA; SF3B1, T-Genes, EA; SIK2, T-
Genes, EA; SKP1, T-Genes, EA; SLC12A5, T-Genes, EA; SLC12A5-AS1, T-Genes, EA; SLC35C2,
T-Genes, EA; SLC35G6, T-Genes, EA; SLC36A1, T-Genes, EA; SLC37A4, T-Genes, EA; SLC9A11,
T-Genes, EA; SLFN11, T-Genes, EA; SMG7, T-Genes, EA; SMIM3, T-Genes, EA; SNAI1, T-Genes,
EA; SNORA32.1, T-Genes, EA; SNORD124, T-Genes, EA; SNORD44, T-Genes, EA; SNORD47, T-
Genes, EA; SNORD56.5, T-Genes, EA; SNORD75, T-Genes, EA; SNORD76, T-Genes, EA;
SNORD77, T-Genes, EA; SNORD78, T-Genes, EA; SNORD79, T-Genes, EA; SNORD80, T-Genes,
EA; SNORD81, T-Genes, EA; snoU13.248, T-Genes, EA; SOCS3, T-Genes, EA; SOCS7, T-Genes,
EA; SOST, T-Genes, EA; SPATA25, T-Genes, EA; SPINT1, T-Genes, EA; STAT1, T-Genes, EA;
STAT4, T-Genes, EA; STAU1, T-Genes, EA; STMN1, T-Genes, EA; SUMO2, T-Genes, EA;
SUPT16H, T-Genes, EA; SYMPK, T-Genes, EA; SYS1, T-Genes, EA; TAF15, T-Genes, EA;
TCEA3, T-Genes, EA; TCF7, T-Genes, EA; TCOF1, T-Genes, EA; TEP1, T-Genes, EA; TK1, T-
Genes, EA; TMEM106A, T-Genes, EA; TMEM106A-AS1, T-Genes, EA; TMEM171, T-Genes, EA;
TMEM176A, T-Genes, EA; TMEM55B, T-Genes, EA; TMEM63C, T-Genes, EA; TMEM75, T-
Genes, EA; TMEM99, T-Genes, EA; TMPRSS13, T-Genes, EA; TNFAIP3, T-Genes, EA; TNFSF18,
T-Genes, EA; TNFSF4, T-Genes, EA; TNIP1, T-Genes, EA; TNNC2, T-Genes, EA; TP53INP2, T-
Genes, EA; TRAF4, T-Genes, EA; TRAPPC4, T-Genes, EA; TREH, T-Genes, EA; TRIM25, T-
Genes, EA; TRPC4AP, T-Genes, EA; TSEN54, T-Genes, EA; TYRO3, T-Genes, EA; U1.108, T-
Genes, EA; U6.1022, T-Genes, EA; U6.1109, T-Genes, EA; U6.1226, T-Genes, EA; U6.1270, T-
Genes, EA; U6.141, T-Genes, EA; U6.305, T-Genes, EA; U6.419, T-Genes, EA; U6.564, T-Genes,
EA; U6.612, T-Genes, EA; U6.98, T-Genes, EA; U6atac.5, T-Genes, EA; U7.110, T-Genes, EA;
UBA52, T-Genes, EA; UBE2C, T-Genes, EA; UPK2, T-Genes, EA; UQCC, T-Genes, EA; VAMP2,
T-Genes, EA; VASH1, T-Genes, EA; VAT1, T-Genes, EA; VAV2, T-Genes, EA; VDAC1, T-Genes,
EA; VPS18, T-Genes, EA; WFDC13, T-Genes, EA; WFDC3, T-Genes, EA; ZBTB37, T-Genes, EA;
ZC3H4, T-Genes, EA; ZDHHC22, T-Genes, EA; ZDHHC7, T-Genes, EA; ZNF219, T-Genes, EA;
ZNF300, T-Genes, EA; ZNF300P1, T-Genes, EA; ZNF335, T-Genes, EA; ZNF436, T-Genes, EA;
ZNF436-AS1, T-Genes, EA; ZPBP, T-Genes, EA; ZPBP2, T-Genes, EA; ZPLD, T-Genes, EA;
BANK1, C-Genes, EA; TMEM39A, C-Genes, EA; ITGAM, C-Genes, EA; UHRF1BP1, C-Genes,
EA; UHRF1BP1, C-Genes, EA; RCAN1, C-Genes, EA; PTPN22, C-Genes, EA; IRF5, E-Gene, EA;
DEF6, E-Gene, EA; SCUBE3, E-Gene, EA; TCP11, E-Gene, EA; ZNF76, E-Gene, EA; ELP3, E-
Gene, EA; NUGGC, E-Gene, EA; AHCYP2, E-Gene, EA; FBXW2, E-Gene, EA; MEGF9, E-Gene,
EA; PSMD5, E-Gene, EA; PSMD5-AS1, E-Gene, EA; TRAF1, E-Gene, EA; MTTP, E-Gene, EA;
TRMT10A, E-Gene, EA; NEDD1, E-Gene, EA; CCDC171, E-Gene, EA; PHTF1, E-Gene, EA; CD44,
E-Gene, EA; CD40, E-Gene, EA; KCNK7, E-Gene, EA; MAP3K11, E-Gene, EA; NEAT1, E-Gene,
EA; SIPA1, E-Gene, EA; C6orf106, E-Gene, EA; SNRPC, E-Gene, EA; UHRF1BP1, E-Gene, EA;
TMEM80, E-Gene, EA; HOXA5, E-Gene, EA; HOXA7, E-Gene, EA; HOTAIRM1, E-Gene, EA;
HOXA-AS2, E-Gene, EA; HOXA1, E-Gene, EA; HOXA2, E-Gene, EA; SKAP2, E-Gene, EA;
ALG1L13P, E-Gene, EA; CLDN23, E-Gene, EA; ENPP7P1, E-Gene, EA; ERI1, E-Gene, EA;
FAM85B, E-Gene, EA; FAM86B3P, E-Gene, EA; MFHAS1, E-Gene, EA; SGK223, E-Gene, EA;
CTB-33G10.11, E-Gene, EA; FCGRT, E-Gene, EA; NOSIP, E-Gene, EA; PRR12, E-Gene, EA;
PRRG2, E-Gene, EA; RCN3, E-Gene, EA; RPS11, E-Gene, EA; TRPM4, E-Gene, EA; CASQ1, E-
Gene, EA; COPA, E-Gene, EA; NCSTN, E-Gene, EA; NHLH1, E-Gene, EA; VANGL2, E-Gene, EA;
C12orf23, E-Gene, EA; RIC8B, E-Gene, EA; RP11-412D9.4, E-Gene, EA; FCGR2C, E-Gene, EA;
IL12RB2, E-Gene, EA; SERBP1, E-Gene, EA; CFH, E-Gene, EA; CFHR1, E-Gene, EA; CFHR3, E-
Gene, EA; CFHR4, E-Gene, EA; KCNT2, E-Gene, EA; 43345, E-Gene, EA; ANO7, E-Gene, EA;
FARP2, E-Gene, EA; HDLBP, E-Gene, EA; STK25, E-Gene, EA; GIMAP8, E-Gene, EA; GTF2H1,
E-Gene, EA; HPS5, E-Gene, EA; LDHA, E-Gene, EA; SAA1, E-Gene, EA; GLT1D1, E-Gene, EA;
SLC15A4, E-Gene, EA; C15orf52, E-Gene, EA; RNA5SP392, E-Gene, EA; UBE2L3, E-Gene, EA;
CCDC116, E-Gene, EA; ABHD6, E-Gene, EA; PDHB, E-Gene, EA; PXK, E-Gene, EA; RPP14, E-
Gene, EA; CARD9, E-Gene, EA; DNLZ, E-Gene, EA; GPSM1, E-Gene, EA; INPP5E, E-Gene, EA;
PMPCA, E-Gene, EA; SDCCAG3, E-Gene, EA; SEC16A, E-Gene, EA; SNAPC4, E-Gene, EA;
AHI1, E-Gene, EA; LINC00271, E-Gene, EA; RP3-388E23.2, E-Gene, EA; POGLUT1, E-Gene, EA;
HTRA1, E-Gene, EA; PLEKHA1, E-Gene, EA; DIRC1, E-Gene, EA; GULP1, E-Gene, EA; ZFP90,
E-Gene, EA; CDH1, E-Gene, EA; SYNGR1, E-Gene, EA; CHRNB2, E-Gene, EA; FAM189B, E-
Gene, EA; IL6R, E-Gene, EA; RP11-350G8.9, E-Gene, EA; SHE, E-Gene, EA; TDRD10, E-Gene,
EA; UBE2Q1-AS1, E-Gene, EA; ACBD3, E-Gene, EA; H3F3A, E-Gene, EA; LEFTY2, E-Gene, EA;
RP11-275114.4, E-Gene, EA; IL7, E-Gene, EA; PKIA, E-Gene, EA; PRKRIRP7, E-Gene, EA;
ZC2HC1A, E-Gene, EA; RPS23P10, E-Gene, EA; AP002387.1, E-Gene, EA; DHCR7, E-Gene, EA;
KRTAP5-10, E-Gene, EA; KRTAP5-7, E-Gene, EA; KRTAP5-8, E-Gene, EA; KRTAP5-9, E-Gene,
EA; NADSYN1, E-Gene, EA; RP11-660L16.2, E-Gene, EA; RP11-684B2.3, E-Gene, EA; DEXI, E-
Gene, EA; HNRNPCP4, E-Gene, EA; PRIM2, E-Gene, EA; AC005702.4, E-Gene, EA; CTD-
2319I12.1, E-Gene, EA; CTD-2319I12.4, E-Gene, EA; NDUFB8P2, E-Gene, EA; RNFT1, E-Gene,
EA; ELL, E-Gene, EA; LRRC25, E-Gene, EA; SSBP4, E-Gene, EA; RP11-380P13.1, E-Gene, EA;
RP11-380P13.2, E-Gene, EA; RP13-497K6.1, E-Gene, EA; ADCK5, E-Gene, EA; CPSF1, E-Gene,
EA; FBXL6, E-Gene, EA; GS1-393G12.14, E-Gene, EA; RP11-661A12.14, E-Gene, EA; SLC39A4,
E-Gene, EA; SLC52A2, E-Gene, EA; COX5A, E-Gene, EA; CPLX3, E-Gene, EA; CSK, E-Gene, EA;
FAM219B, E-Gene, EA; ISLR2, E-Gene, EA; LMAN1L, E-Gene, EA; MPI, E-Gene, EA; PPCDC, E-
Gene, EA; SCAMP2, E-Gene, EA; ULK3, E-Gene, EA; EPB41L2, E-Gene, EA; SMLR1, E-Gene,
EA; ART3, E-Gene, EA; FAM47E, E-Gene, EA; NAAA, E-Gene, EA; SCARB2, E-Gene, EA; FBF1,
E-Gene, EA; GALK1, E-Gene, EA; MRPL38, E-Gene, EA; TEN1, E-Gene, EA; TRIM47, E-Gene,
EA; TRIM65, E-Gene, EA; UNC13D, E-Gene, EA; WBP2, E-Gene, EA; ADPRH, E-Gene, EA;
APOE, E-Gene, EA; CLPTM1, E-Gene, EA; ALDH2, E-Gene, EA; MAPKAPK5, E-Gene, EA;
NIPAL3, E-Gene, EA; RP4-594I10.3, E-Gene, EA; FOXP4, E-Gene, EA; RP11-328M4.2, E-Gene,
EA; ASIP, E-Gene, EA; CBFA2T2, E-Gene, EA; E2F1, E-Gene, EA; PXMP4, E-Gene, EA; RP1-
63M2.6, E-Gene, EA; CAMKK2, E-Gene, EA; AC012370.3, E-Gene, EA; C10orf107, E-Gene, EA;
RP11-809M12.1, E-Gene, EA; TMEM26, E-Gene, EA; AC009061.1, E-Gene, EA; ATP6V0D1, E-
Gene, EA; CTD-2012K14.7, E-Gene, EA; CTRL, E-Gene, EA; DPEP3, E-Gene, EA; DUS2, E-Gene,
EA; ELMO3, E-Gene, EA; ESRP2, E-Gene, EA; GFOD2, E-Gene, EA; HSD11B2, E-Gene, EA;
LCAT, E-Gene, EA; LRRC36, E-Gene, EA; NFATC3, E-Gene, EA; NUTF2, E-Gene, EA; PARD6A,
E-Gene, EA; PLA2G15, E-Gene, EA; PRMT7, E-Gene, EA; PSMB10, E-Gene, EA; RANBP10, E-
Gene, EA; RP11-67A1.2, E-Gene, EA; RP11-96D1.5, E-Gene, EA; RP11-96D1.7, E-Gene, EA;
SLC12A4, E-Gene, EA; SLC7A6, E-Gene, EA; TSNAXIP1, E-Gene, EA; ZDHHC1, E-Gene, EA;
KIF24, E-Gene, EA; NUDT2, E-Gene, EA; UBAP1, E-Gene, EA; AC026740.1, E-Gene, EA; CEP72,
E-Gene, EA; CTD-2589H19.6, E-Gene, EA; SLC9A3, E-Gene, EA; ZDHHC11, E-Gene, EA; RP11-
255B23.4, E-Gene, EA; CTD-2376I4.2, E-Gene, EA; CTD-2631K10.1, E-Gene, EA; FCHO2, E-
Gene, EA; AC064852.5, E-Gene, EA; C2orf82, E-Gene, EA; NGEF, E-Gene, EA; AGAP5, E-Gene,
EA; FUT11, E-Gene, EA; MYOZ1, E-Gene, EA; PLAU, E-Gene, EA; RP11-574K11.29, E-Gene, EA;
SEC24C, E-Gene, EA; GGA3, E-Gene, EA; LLGL2, E-Gene, EA; MIF4GD, E-Gene, EA; MRPS7, E-
Gene, EA; NUP85, E-Gene, EA; FADS1, E-Gene, EA; FADS2, E-Gene, EA; FADS3, E-Gene, EA;
MYRF, E-Gene, EA; RP11-703H8.7, E-Gene, EA; TMEM258, E-Gene, EA; HMGCLL1, E-Gene,
EA; RASIP1, E-Gene, EA; FUT2, E-Gene, EA; RGS1, E-Gene, EA; RGS18, E-Gene, EA; RP5-
1011O1.2, E-Gene, EA; WDFY4, E-Gene, EA; LYN, E-Gene, EA; RP11-446E9.1, E-Gene, EA;
RPS20, E-Gene, EA; C6orf183, E-Gene, EA; CCDC162P, E-Gene, EA; DGKQ, E-Gene, EA; GAK,
E-Gene, EA; IDUA, E-Gene, EA; SLC26A1, E-Gene, EA; ARNT2, E-Gene, EA; RP11-210M15.2, E-
Gene, EA; RP11-379K22.2, E-Gene, EA; SPATS2L, E-Gene, EA; NCK2, E-Gene, EA; CDK3, E-
Gene, EA; RP11-552F3.9, E-Gene, EA; SLC2A13, E-Gene, EA; AC079630.2, E-Gene, EA; LRRK2,
E-Gene, EA; RP11-476D10.1, E-Gene, EA; WFS1, E-Gene, EA; GSDMA, E-Gene, EA; GSDMB, E-
Gene, EA; ORMDL3, E-Gene, EA; ITIH2, E-Gene, EA; AC087650.1, E-Gene, EA; ARL4D, E-Gene,
EA; BRCA1, E-Gene, EA; CTC-501O10.1, E-Gene, EA; CTD-3193K9.3, E-Gene, EA; DHX8, E-
Gene, EA; IFI35, E-Gene, EA; LINC00854, E-Gene, EA; LINC00910, E-Gene, EA; MIR2117, E-
Gene, EA; NBR1, E-Gene, EA; NBR2, E-Gene, EA; PTRF, E-Gene, EA; RND2, E-Gene, EA;
TMEM106A, E-Gene, EA; TUBG2, E-Gene, EA; VAT1, E-Gene, EA; FBXO40, E-Gene, EA;
GOLGB1, E-Gene, EA; HCLS1, E-Gene, EA; IQCB1, E-Gene, EA; SLC15A2, E-Gene, EA; GALC,
E-Gene, EA; FAM203B, E-Gene, EA; FAM83H, E-Gene, EA; NRBP2, E-Gene, EA; PUF60, E-Gene,
EA; RP11-429J17.5, E-Gene, EA; RP11-429J17.8, E-Gene, EA; SCRIB, E-Gene, EA; FAM193A, E-
Gene, EA; SH3BP2, E-Gene, EA; ZFYVE28, E-Gene, EA; RP11-63K6.5, E-Gene, EA; ARL14EP, E-
Gene, EA; MPPED2, E-Gene, EA; RP4-710M3.1, E-Gene, EA; C11orf1, E-Gene, EA; COLCA1, E-
Gene, EA; COLCA2, E-Gene, EA; CRYAB, E-Gene, EA; LAYN, E-Gene, EA; PPIHP1, E-Gene, EA;
PPP2R1B, E-Gene, EA; SIK2, E-Gene, EA; CTD-2587H24.10, E-Gene, EA; HSPBP1, E-Gene, EA;
PPP6R1, E-Gene, EA; HSD17B1P1, E-Gene, EA; DCPS, E-Gene, EA; FAM118B, E-Gene, EA;
FOXRED1, E-Gene, EA; RP11-50B3.2, E-Gene, EA; RPUSD4, E-Gene, EA; CCDC36, E-Gene, EA;
CDHR4, E-Gene, EA; DOCK3, E-Gene, EA; FAM212A, E-Gene, EA; HYAL3, E-Gene, EA; RBM6,
E-Gene, EA; AP003774.1, E-Gene, EA; CCDC88B, E-Gene, EA; PLCB3, E-Gene, EA; PPP1R14B,
E-Gene, EA; RP11-783K16.5, E-Gene, EA; RPS6KA4, E-Gene, EA; CTNNAL1, E-Gene, EA;
IKBKAP, E-Gene, EA; TMEM245, E-Gene, EA; CCDC170, E-Gene, EA; ZBTB2, E-Gene, EA;
EPS8L2, E-Gene, EA; HRAS, E-Gene, EA; AC087269.1, E-Gene, EA; LINC00599, E-Gene, EA;
PPP1R3B, E-Gene, EA; RP11-115J16.2, E-Gene, EA; RP11-375N15.2, E-Gene, EA; RP11-62H7.2,
E-Gene, EA; TNKS, E-Gene, EA; EVI5, E-Gene, EA; RP1-20N18.4, E-Gene, EA; SPRR1B, E-Gene,
EA; SPRR2B, E-Gene, EA; SPRR2D, E-Gene, EA; SPRR2G, E-Gene, EA; LCE1D, E-Gene, EA;
LCE1E, E-Gene, EA; LCE3C, E-Gene, EA; SMCP, E-Gene, EA; RP1-111C20.4, E-Gene, EA; CTC-
458A3.1, E-Gene, EA; NDRG1, E-Gene, EA; ST3GAL1, E-Gene, EA; JAZF1, E-Gene, EA; JAZF1-
AS1, E-Gene, EA; HEATR3, E-Gene, EA; RP11-429P3.3, E-Gene, EA; CD226, E-Gene, EA;
KRT222, E-Gene, EA; KRT24, E-Gene, EA; SMARCE1, E-Gene, EA; BCKDK, E-Gene, EA;
C16orf93, E-Gene, EA; HSD3B7, E-Gene, EA; ITGAX, E-Gene, EA; KAT8, E-Gene, EA; PRR14, E-
Gene, EA; PRSS36, E-Gene, EA; RNF40, E-Gene, EA; STX1B, E-Gene, EA; STX4, E-Gene, EA;
ZNF668, E-Gene, EA; CTD-2587H24.5, E-Gene, EA; DNAAF3, E-Gene, EA; TNNI3, E-Gene, EA;
ASCC2, E-Gene, EA; NIPSNAP1, E-Gene, EA; RNF215, E-Gene, EA; RP1-130H16.16, E-Gene, EA;
THOC5, E-Gene, EA; DLX2, E-Gene, EA; MLTK, E-Gene, EA; ICAM4, E-Gene, EA; CCDC101, E-
Gene, EA; CDC37P1, E-Gene, EA; CLN3, E-Gene, EA; EIF3C, E-Gene, EA; EIF3CL, E-Gene, EA;
IL27, E-Gene, EA; LAT, E-Gene, EA; MIR4721, E-Gene, EA; NFATC2IP, E-Gene, EA; NPIPB6, E-
Gene, EA; NPIPB7, E-Gene, EA; NUPR1, E-Gene, EA; RP11-1348G14.4, E-Gene, EA; RP11-
1348G14.6, E-Gene, EA; RP11-22P6.2, E-Gene, EA; RP11-435I10.5, E-Gene, EA; SBK1, E-Gene,
EA; SH2B1, E-Gene, EA; SPNS1, E-Gene, EA; SULT1A1, E-Gene, EA; SULT1A2, E-Gene, EA;
TUFM, E-Gene, EA; COMMD7, E-Gene, EA; DNMT3B, E-Gene, EA; PIGK, E-Gene, EA;
CDC42BPA, E-Gene, EA; SNAP47, E-Gene, EA; F5, E-Gene, EA; SELL, E-Gene, EA; C3orf18, E-
Gene, EA; CACNA2D2, E-Gene, EA; CYB561D2, E-Gene, EA; GRM2, E-Gene, EA; HEMK1, E-
Gene, EA; HYAL1, E-Gene, EA; MANF, E-Gene, EA; MAPKAPK3, E-Gene, EA; RP11-804H8.6, E-
Gene, EA; RPL29, E-Gene, EA; TEX264, E-Gene, EA; U73166.2, E-Gene, EA; NEURL2, E-Gene,
EA; PLTP, E-Gene, EA; C5orf52, E-Gene, EA; CTB-109A12.1, E-Gene, EA; CTB-47B11.3, E-Gene,
EA; FNDC9, E-Gene, EA; CCDC18, E-Gene, EA; DR1, E-Gene, EA; RP4-713B5.2, E-Gene, EA;
RP4-717I23.3, E-Gene, EA; TMED5, E-Gene, EA; IFT88, E-Gene, EA; IL17D, E-Gene, EA; RNU2-
7P, E-Gene, EA; SLC35E1P1, E-Gene, EA; XPO4, E-Gene, EA; FAM167A, E-Gene, EA; LACC1, E-
Gene, EA; C6orf3, E-Gene, EA; KIAA1919, E-Gene, EA; REV3L, E-Gene, EA; RP5-1112D6.8, E-
Gene, EA; TRAF3IP2, E-Gene, EA; UNC5B, E-Gene, EA; UNC5B-AS1, E-Gene, EA; OAS1, E-
Gene, EA; OAS2, E-Gene, EA; OAS3, E-Gene, EA; RP1-71H24.1, E-Gene, EA; RP11-109L13.1, E-
Gene, EA; FAM98C, E-Gene, EA; C11orf35, E-Gene, EA; RP11-496I9.1, E-Gene, EA; RP11-
754B17.1, E-Gene, EA; SMO, E-Gene, EA; TNPO3, E-Gene, EA; TSPAN33, E-Gene, EA; ARMC7,
E-Gene, EA; NT5C, E-Gene, EA; RP11-649A18.12, E-Gene, EA; SLC25A19, E-Gene, EA;
AC004490.1, E-Gene, EA; AMH, E-Gene, EA; AP3D1, E-Gene, EA; DOT1L, E-Gene, EA;
IZUMO4, E-Gene, EA; MAP3K19, E-Gene, EA; MCM6, E-Gene, EA; ZRANB3, E-Gene, EA;
CCNO, E-Gene, EA; CDC20B, E-Gene, EA; DHX29, E-Gene, EA; GZMA, E-Gene, EA; MCIDAS,
E-Gene, EA; RNF138P1, E-Gene, EA; RP11-506H20.1, E-Gene, EA; SKIV2L2, E-Gene, EA; CTD-
2260A17.1, E-Gene, EA; ERAP1, E-Gene, EA; ERAP2, E-Gene, EA; RMI2, E-Gene, EA; MX1, E-
Gene, EA; CLEC10A, E-Gene, EA; TNFRSF21, E-Gene, EA; IFIT1, E-Gene, EA; HERC5, E-Gene,
EA; CLEC4A, E-Gene, EA; IFI6, E-Gene, EA; CCDC50, E-Gene, EA; SCAMP5, E-Gene, EA;
C1QB, E-Gene, EA; IFI44L, E-Gene, EA; TBX20, E-Gene, EA; ADO, P-Gene, EA; BANK1, P-Gene,
EA; BLK, P-Gene, EA; CD44, P-Gene, EA; ETS1, P-Gene, EA; FASLG, P-Gene, EA; IRF4, P-Gene,
EA; IRF5, P-Gene, EA; JAZF1, P-Gene, EA; LOC100506023, P-Gene, EA; MIR146A, P-Gene, EA;
PDHX, P-Gene, EA; PTTG1, P-Gene, EA; RASGRP3, P-Gene, EA; STAT4, P-Gene, EA; TNFSF4,
P-Gene, EA; TNIP1, P-Gene, EA; TNPO3, P-Gene, EA; TPI1P2, P-Gene, EA; WDFY4, P-Gene, EA;
ZNF365, P-Gene, EA; ABTB2, T-Genes, EA; AC004231.2, T-Genes, EA; AC011363.1, T-Genes,
EA; AC025594.1, T-Genes, EA; ACOX2, T-Genes, EA; ACTR2, T-Genes, EA; AL109947.1, T-
Genes, EA; AL138810.1, T-Genes, EA; APIP, T-Genes, EA; ARMC2, T-Genes, EA; BAG2, T-Genes,
EA; BLK, T-Genes, EA; C1QTNF2, T-Genes, EA; C5orf54, T-Genes, EA; C8orf12, T-Genes, EA;
CALU, T-Genes, EA; CAPRIN1, T-Genes, EA; CAT, T-Genes, EA; CCDC136, T-Genes, EA;
CCDC162P, T-Genes, EA; CCNJL, T-Genes, EA; CCR7, T-Genes, EA; CD164, T-Genes, EA; CD44,
T-Genes, EA; CDC6, T-Genes, EA; CDC73, T-Genes, EA; CEP57L1, T-Genes, EA; CEP68, T-Genes,
EA; CSK, T-Genes, EA; CTC-529G1.1, T-Genes, EA; DCPS, T-Genes, EA; DENND6A, T-Genes,
EA; DST, T-Genes, EA; EHF, T-Genes, EA; EIF1, T-Genes, EA; ENSG00000243295, T-Genes, EA;
ENSG00000254694, T-Genes, EA; ENSG00000254771, T-Genes, EA; ENSG00000254790, T-Genes,
EA; ENSG00000254833, T-Genes, EA; ENSG00000272182, T-Genes, EA; FAM107A, T-Genes, EA;
FAM118B, T-Genes, EA; FAM164A, T-Genes, EA; FAM167A, T-Genes, EA; FAM71F2, T-Genes,
EA; FLNB, T-Genes, EA; FLNB-AS1, T-Genes, EA; FOXRED1, T-Genes, EA; GJD3, T-Genes, EA;
HTD2, T-Genes, EA; IL7, T-Genes, EA; IRF5, T-Genes, EA; KCTD6, T-Genes, EA; KIAA1586, T-
Genes, EA; KLHL10, T-Genes, EA; KRT12, T-Genes, EA; KRT13, T-Genes, EA; KRT14, T-Genes,
EA; KRT15, T-Genes, EA; KRT16, T-Genes, EA; KRT17, T-Genes, EA; KRT19, T-Genes, EA;
KRT20, T-Genes, EA; KRT222, T-Genes, EA; KRT23, T-Genes, EA; KRT24, T-Genes, EA; KRT25,
T-Genes, EA; KRT26, T-Genes, EA; KRT27, T-Genes, EA; KRT28, T-Genes, EA; KRT31, T-Genes,
EA; KRT33A, T-Genes, EA; KRT37, T-Genes, EA; KRT38, T-Genes, EA; KRT40, T-Genes, EA;
KRT42P, T-Genes, EA; KRT9, T-Genes, EA; KRTAP1-5, T-Genes, EA; KRTAP16-1, T-Genes, EA;
KRTAP17-1, T-Genes, EA; KRTAP2-3, T-Genes, EA; KRTAP2-4, T-Genes, EA; KRTAP3-1, T-
Genes, EA; KRTAP4-12, T-Genes, EA; KRTAP4-6, T-Genes, EA; KRTAP9-2, T-Genes, EA;
KRTAP9-6, T-Genes, EA; KRTAP9-9, T-Genes, EA; LDLRAD3, T-Genes, EA; LMO2, T-Genes,
EA; LOC147093, T-Genes, EA; MICAL1, T-Genes, EA; MIR1278, T-Genes, EA; MIR146A, T-
Genes, EA; MIR4513, T-Genes, EA; MSL1, T-Genes, EA; OPN1SW, T-Genes, EA; PDE12, T-Genes,
EA; PDHX, T-Genes, EA; PKIA, T-Genes, EA; PPIL6, T-Genes, EA; PRIM2, T-Genes, EA; PXK, T-
Genes, EA; RAB1A, T-Genes, EA; RAB23, T-Genes, EA; RARA, T-Genes, EA; RGS1, T-Genes,
EA; RNU4-86P, T-Genes, EA; RNU7-16P, T-Genes, EA; RP1-68D18.3, T-Genes, EA; RP11-
148O21.3, T-Genes, EA; RP11-148O21.4, T-Genes, EA; RP11-286H14.8, T-Genes, EA; RP11-
309L24.2, T-Genes, EA; RP4-607I7.1, T-Genes, EA; RP4-683L5.1, T-Genes, EA; RP5-919F19.5, T-
Genes, EA; RPP14, T-Genes, EA; RPUSD4, T-Genes, EA; SLC1A2, T-Genes, EA; SLU7, T-Genes,
EA; SMARCE1, T-Genes, EA; SMPD2, T-Genes, EA; SNORA74.1, T-Genes, EA; snoU109.3, T-
Genes, EA; snoU13.218, T-Genes, EA; snoU13.89, T-Genes, EA; SPRED2, T-Genes, EA; TNPO3, T-
Genes, EA; TOP2A, T-Genes, EA; TPI1P2, T-Genes, EA; TSPAN33, T-Genes, EA; U6.116, T-Genes,
EA; U6.85, T-Genes, EA; UVRAG, T-Genes, EA; XKR6, T-Genes, EA; ZBTB24, T-Genes, EA;
ZC2HC1A, T-Genes, EA; ZNF451, T-Genes, EA; CTC-493P15.3, T-Genes, EA; FLNC, T-Genes,
EA; U6.1291, T-Genes, EA;

TABLE 15A
EA Immunochip MCODE clusters (FIG. 9). Listed by: MCODE cluster, gene;

1, KRT12; 1, KRT13; 1, KRT16; 1, KRT17; 1, KRT19; 1, KRT23; 1, KRT31; 1, KRT33A; 1, KRT37;
1, KRT38; 1, KRT40; 2, BCL6; 2, CASP1; 2, CR1; 2, CR2; 2, IFI16; 2, IFT88; 2, IL1R1; 2, PTPN22;
2, VAV2; 2, ACOX2; 2, ACTR2; 2, ADORA3; 2, AMPH; 2, ARPC2; 2, ARRB2; 2, ASB16; 2, ASIP;
2, ATXN1; 2, BAG2; 2, CASP10; 2, CASP8; 2, CASR; 2, CCDC84; 2, CCL17; 2, CCR10; 2, CCR9;
2, COMMD7; 2, CRYAB; 2, CXCL16; 2, CXCR1; 2, DLL4; 2, DNM3; 2, EEPD1; 2, EIF5A; 2,
ELP3; 2, ENG; 2, ENTHD1; 2, EXOC2; 2, FBXO40; 2, FBXW2; 2, FCGR2A; 2, FCHO1; 2, FLNB;
2, GAK; 2, GRM4; 2, GRM7; 2, GTF3C3; 2, HERC5; 2, HSPBP1; 2, HSPD1; 2, HTR5A; 2, IFI30; 2,
IFI35; 2, IFI44L; 2, IFI6; 2, IFIH1; 2, IFIT1; 2, IKBKAP; 2, IL18BP; 2, IL27; 2, IRF4; 2, IRF7; 2,
IRGM; 2, ITCH; 2, KCTD6; 2, KDM4B; 2, MKKS; 2, MRPL45; 2, MRPS7; 2, MX1; 2, MYOZ1; 2,
NBR1; 2, NEDD4L; 2, NEURL2; 2, NFATC2IP; 2, NLRX1; 2, NOTCH2; 2, OAS2; 2, OR11H4; 2,
OR6S1; 2, OTUD3; 2, PAPOLG; 2, PARD6A; 2, PPIL3; 2, PPIL6; 2, PTGES3L; 2, PUF60; 2, QARS;
2, RBM17; 2, RBM22; 2, RNF215; 2, RPAP1; 2, RPS25; 2, SAA1; 2, SCARB2; 2, SF3B1; 2,
SH3PXD2B; 2, SKI; 2, SKIV2L2; 2, SLC22A11; 2, SMO; 2, SNAP47; 2, SOCS3; 2, SPPL2C; 2,
SRRT; 2, SRSF6; 2, STAT1; 2, SYMPK; 2, SYN2; 2, SYN3; 2, SYNJ2; 2, TMEM55B; 2, TNFAIP3;
2, TRIM25; 2, TRIM63; 2, TRMT11; 2, TTC28; 2, UBA52; 2, UBAP1; 2, UBE2C; 2, UBE2K; 2,
VAMP2; 2, WBP2; 2, XPO4; 2, ZBP1; 3, AHI1; 3, CEP192; 3, CEP250; 3, CEP72; 3, CEP97; 3,
FBF1; 3, KIF24; 3, LPHN3; 3, MRPL38; 3, NEDD1; 4, ATP5H; 4, CHCHD2; 4, DNMT3L; 4,
EIF3C; 4, EIF3CL; 4, EIF6; 4, ERCC1; 4, FAM86C1; 4, ILF3; 4, INO80; 4, INO80D; 4, IPO5; 4,
KCNB1; 4, KCNG1; 4, KCTD19; 4, NUDT2; 4, NUFIP2; 4, PARP2; 4, PELP1; 4, RAD23B; 4,
RPL19; 4, RPL24; 4, RPL27; 4, RPL29; 4, RPP14; 4, RPS11; 4, SAMD15; 4, SMG7; 4, SUMO2; 4,
TEP1; 4, TUFM; 4, UTP23; 5, DGKQ; 5, FLT1; 5, GPR39; 5, IPMK; 5, ITPR2; 5, P2RY6; 5, PLCB1;
5, PLCB2; 5, PLCB3; 7, IL6R; 7, APEX1; 7, CD2; 7, CD3E; 7, CD5; 7, GIMAP1; 7, GIMAP4; 7,
GIMAP5; 7, GIMAP6; 7, GIMAP7; 7, GIMAP8; 7, GZMA; 7, HCLS1; 7, ICOS; 7, IFNG; 7, IL10; 7,
IL10RA; 7, IL17D; 7, IL2; 7, IL21; 7, IL7; 7, JAK3; 7, RNASE6; 8, ADNP; 8, CHD7; 8, CPSF3L; 8,
DHX8; 8, ELL; 8, RPAP2; 8, RTF1; 8, SNAPC4; 8, SUPT16H; 8, ZC3H4; 10, ADCYAP1R1; 10,
CA10; 10, CRHR1; 10, CTXN3; 10, FAM163B; 10, NEUROD6; 10, NGB; 10, POMT2; 10,
RBFOX1; 10, RLN2; 11, APLP1; 11, ATF6; 11, CD86; 11, CENPL; 11, CENPW; 11, CNKSR1; 11,
CPPED1; 11, CTSA; 11, CUX1; 11, DCTN4; 11, DDX27; 11, DENND1A; 11, DENND6A; 11, F5;
11, GDI2; 11, GEMIN4; 11, GOLGB1; 11, H2AFX; 11, HSPE1; 11, IL17RD; 11, IPO8; 11, ITIH2;
11, IZUMO4; 11, KIF18A; 11, KIF1A; 11, KIF1C; 11, KIF5A; 11, KNSTRN; 11, LAMC1; 11,
LAMC2; 11, LAT; 11, LEFTY2; 11, MAD1L1; 11, MAPT; 11, NCOA5; 11, NEK6; 11, NEK7; 11,
NSF; 11, NUP85; 11, OIP5; 11, PDS5A; 11, PEBP1; 11, PPP2R1B; 11, RAB1A; 11, RHOB; 11,
SDC1; 11, STAU1; 11, STK25; 11, STMN1; 11, TK1; 11, TNC; 11, TRAPPC4; 11, TRIM24; 11,
UNC13D; 11, UNC5B; 11, VAT1; 11, WFS1; 11, ZC3HAV1; 12, APOE; 12, HDLBP; 12, LCAT; 12,
PCYOX1; 12, PLA2G15; 12, PLTP; 12, SEC24C; 13, CARM1; 13, H3F3B; 13, MED1; 13, MED24;
13, NCOA6; 13, NPAS2; 13, RARB; 13, RGL1; 13, RORA; 13, TNFRSF21; 14, CLEC4D; 14,
CLEC6A; 14, FCER1G; 14, LAMTOR1; 14, ORMDL3; 14, PDGFRB; 14, PLAU; 15, CD38; 15,
NT5C; 15, NT5C3B; 15, NT5E; 15, PDE3B; 15, PNP; 17, ANAPC15; 17, BRCA1; 17, CCND1; 17,
CDC20B; 17, CDH1; 17, CEBPB; 17, E2F1; 17, ETV6; 17, FAM83H; 17, GTF2H1; 17, GTF2H5; 17,
HIF3A; 17, NUSAP1; 17, PSMB10; 17, PSMD5; 17, RAD51; 17, SMARCA4; 17, TNFSF13B; 17,
TRAF3; 17, USP14; 18, CD74; 18, CHIT1; 18, CTSH; 18, LTA4H; 18, YPEL5; 19, AP3B2; 19,
AP3D1; 19, ARL17B; 19, GGA3; 19, VPS18; 22, CCDC101; 22, DR1; 22, FOXR1; 22, MORF4L1;
22, RNFT1; 22, TADA2B;

TABLE 15B
AsA Immunochip MCODE clusters (FIG. 9). Listed by: MCODE cluster, gene;

1, HIST1H4G; 1, PHF2; 1, TSPYL1; 1, HIST1H2AG; 1, HIST1H1C; 1, HFE; 1, HIST1H4I; 1,
HIST1H4H; 1, HIST1H1E; 1, HIST1H4C; 1, CHAMP1; 1, H2AFB1; 1, HIST1H3E; 1,
HIST1H3H; 1, HIST1H3G; 1, HIST1H3J; 1, HIST1H2AC; 1, HIST1H2BB; 1, HIST1H2BO; 1,
HIST1H2BJ; 1, HIST1H2BK; 1, HIST1H2AD; 1, POLR2E; 2, ZNF768; 2, UBQLN4; 2, UBL4A;
2, NAA25; 2, TFAM; 2, OXA1L; 2, NUP88; 2, POM121C; 2, SREBF1; 2, IPO9; 2, HIST1H1T;
2, TMA7; 2, TAF7; 2, HCFC1; 2, HDAC9; 2, KMT2B; 2, KLK3; 2, KLK2; 2, GPN3; 2, FLI1; 2,
HMGN1; 2, MYSM1; 2, RPSA; 2, RPS10; 2, RPS24; 2, EIF1AD; 2, NAB1; 2, EGR2; 2,
KPNB1; 2, HDAC7; 2, SETD8; 2, SLC25A6; 2, VBP1; 2, SRP54; 2, REST; 2, RPL10; 2,
PARK2; 2, CENPQ; 2, NAA10; 2, TAF11; 2, CCNT2; 2, MRPL4; 2, C12orf65; 2, RPL14; 2,
RPL3; 2, RPL6; 2, POLR2B; 2, RPL10A; 2, HIST1H2BN; 2, PSMA6; 2, EIF3E; 2, POU2F1; 2,
SEC61A2; 2, SETD1A; 2, BRCC3; 2, DARS; 2, PA2G4; 2, POLR1D; 3, TLR5; 3, MGAT3; 3,
IL1A; 3, F11R; 3, PYDC1; 3, PYCARD; 3, ERC1; 3, NOD2; 3, TAB1; 3, CYLD; 3, PRKCI; 3,
TNIP2; 3, BRD7; 3, REL; 3, TLR4; 3, PPP2R3B; 3, BCL10; 3, MTCP1; 3, LAMTOR2; 3,
PIK3CG; 3, NOTCH1; 3, PFKL; 3, ERBB3; 3, MAP3K8; 3, CDC37; 3, GNG2; 3, ADCY7; 3,
IRAK1; 3, ADCK3; 4, PGGT1B; 4, WDR73; 4, POLB; 4, XRCC1; 4, RAD9B; 4, RFC2; 4,
DNA2; 4, RAD1; 4, MPP1; 4, RTEL1; 4, RPP25; 4, POLR3A; 4, FEN1; 4, RMI1; 4, FANCE; 4,
C19orf40; 4, NBN; 4, DKC1; 4, TERT; 4, AKT1; 4, KRI1; 4, DDX55; 4, ABT1; 5, XRCC4; 5,
XRCC6BP1; 5, PMS1; 6, IL1B; 6, IL1RN; 6, SIGIRR; 6, IL18R1; 6, ICAM1; 6, LIF; 6, IL4; 6,
NTRK2; 6, WWOX; 6, CEP290; 6, FGFR1OP; 6, CPSF6; 6, BCR; 6, FUS; 6, STAT3; 7,
KCNU1; 7, KCNMB2; 7, KCNMB4; 9, MCU; 9, MRPL39; 9, NDUFA9; 9, COX6B1; 9,
UQCR10; 9, CISD2; 9, NDUFAB1; 9, COX6A2; 9, CISD1; 9, COX17; 9, VDAC3; 9, HINT1;
10, TBCD; 10, JAK2; 10, ATG7; 10, TRAK1; 10, VWF; 10, CLCN6; 10, AGTRAP; 11, IL36RN;
11, IL1RAPL1; 11, IL36B; 11, IL36A; 11, IL1F10; 12, ADAMTSL1; 12, ADAMTSL3; 12,
ADAMTS3; 12, ADAMTS9; 12, ACAN; 13, RAI14; 13, SCN7A; 13, SCN4B; 13, CDCA2; 13,
F8; 13, GRIN3B; 13, CNIH3; 13, GRIK4; 13, RPS6KA2; 13, PRKD2; 13, PRKD1; 13, BATF;
13, CEBPG; 13, WDR92; 13, ATF4; 13, TRIB3; 13, PPP1CC; 13, AKAP11; 15, WNT4; 15,
PLXNB1; 15, PLXNB3; 15, LIMK1; 15, HMHA1; 15, PLXNA3; 15, NRP1; 15, DPYSL4; 15,
PARD3; 15, ELMO1; 15, RAC2; 16, P4HA2; 16, SPP1; 16, COL9A1; 16, PLOD1; 16, RUNX3;
16, SYK; 16, SCARB1; 16, MSR1; 16, SDC4; 16, GPC5; 16, C6; 16, KMO; 16, COL4A2; 16,
IBSP; 16, ALPL; 16, ITIH2; 16, APOB; 16, ALB; 16, PEX3; 16, ACSL6; 17, TCERG1; 17,
SNRNP35; 17, NOA1; 17, ORC3; 17, XPO1; 17, UTP20; 17, ZMAT5; 17, RARS2; 17,
DALRD3; 17, SF3B2; 17, U2AF1L4; 17, CHERP; 17, WDR6; 17, PSME3; 17, TARS; 17,
DPYD; 17, TDRD12; 17, SNRPA1; 17, VIMP; 17, ADA; 17, BRIX1; 17, PES1; 17, LTV1; 17,
WDR18; 19, MGST3; 19, GSTA1; 19, GPX3; 21, RASGRP1; 21, RASGRF2; 21, CACNA1S; 21,
PPP2R3C; 21, CACNG3; 21, ADAM23; 22, USP34; 22, PEX7; 22, PEX13; 22, IKBKE; 22,
ERCC8; 22, KEAP1; 22, SLC22A5; 22, UBE2E3; 22, UBE2D3; 22, EIF2AK1; 22, ASB7; 22,
ASB15; 22, COMMD8; 22, ASB1; 22, VPS29; 22, IKBKB; 22, UBE2D1; 22, NFKB1; 23,
RASSF5; 23, MYO15A; 23, ITGAD; 23, NDST3; 23, NDST4; 23, HS3ST2; 23, HS3ST1; 23,
L1CAM; 23, EFNA5; 23, NCAPG; 23, CD9; 23, ITGB1; 23, LAMB2; 23, NCKIPSD; 23,
ARHGEF7; 23, HSPG2; 23, EPHB2; 23, EGFR; 23, PRKCB; 23, MMP13; 25, IL21R; 25,
IL11RA; 25, CCL7; 25, CX3CR1; 25, CCL15; 25, CCL8; 25, C1QC; 25, C1QA; 28, MUT; 28,
NIT1; 28, FHIT; 28, MCCC1; 28, AUH; 29, TRAF3IP1; 29, IFT81; 29, DYNC2LI1; 29,
DYNC1I2; 29, DNAH10; 29, C1orf106; 29, GALNT2; 29, KIF5B; 29, ACTR1A; 30, MECP2;
30, LAT2; 30, SYT1; 30, MOBP; 30, AMT; 30, CNDP1; 30, ALDH9A1; 30, GAD1; 30,
ADSSL1; 31, NPR3; 31, PKD2; 31, CNGB1; 31, CNGA1; 31, RAB3IP; 31, CCDC146; 40,
SKIL; 40, E2F2; 40, CDC25A; 40, CDC16; 40, ANAPC16; 40, TRIM33; 40, CBX7; 40,
ANAPC1; 40, CDKN1A; 41, CHCHD7; 41, CL4BL; 41, CPS1; 41, LRRK1; 41, MFN2; 41,
MICU1; 41, NME6; 41, OSGEPL1; 41, TIMM7A;

TABLE 15C
Shared Immunochip MCODE clusters (FIG. 9). Listed by: MCODE cluster, gene;

1, IL2RA; 1, STAT4; 1, HSPA6; 1, CD44; 1, ANK3; 1, IRF8; 1, IRF5; 1, LYN; 1, ITGAX; 1,
FCGR2A; 1, FLNC; 1, CCL22; 1, CXCR5; 1, GPR29; 1, CCR7; 1, HYAL3; 1, DHCR7; 1,
SMARCE1; 1, GRM2; 1, OAS3; 2, KRT28; 2, KRT25; 2, KRT26; 2, KRT27; 2, KRT15; 2, KRT24;
3, TCF7; 3, CTLA4; 3, IKZF1; 3, BANK1; 3, UHRF1BP1; 3, BLK; 3, FAM167A; 3, TNIP1; 3,
ITGAM; 3, RASGRP3; 3, UBE2L3; 3, XKR6; 3, CDH17; 3, CSK; 3, KRT9; 3, CD40; 3, SLC15A4;
3, FDFT1; 3, PTPRC; 3, PPP2CA; 3, CADM1; 3, TNPO3; 3, NOS2; 3, CX3CL1; 3, CD80; 3, CD226;
3, SLAMF7; 3, KRT14; 3, KRT20; 3, HYAL1; 3, CR1; 3, ETS1; 3, JAZF1; 3, EIF1; 3, SIPA1; 5,
GINS2; 5, CCNJL; 5, C3orf18; 5, CDC6; 5, CD6; 5, REV3L; 5, FBXW2; 5, RMI2; 5, SKP1; 5,
PTTG1; 5, TOP2A; 5, MCM6; 5, HEMK1; 5, ZRANB3; 6, C7orf72; 6, ZPBP; 6, THSD7A; 6,
FIGNL1; 6, CCDC36; 6, VANGL2; 6, COBL; 6, KLHL10; 7, ALDH2; 7, MTHFR; 9, CAT; 9,
COPA; 9, DGKH; 9, RPTOR; 9, WDFY4; 9, CLEC16A; 9, BCAR1; 9, RGS1; 9, C1QB; 9, PLEK; 9,
NCF2; 9, CLECL1; 9, GRB10; 9, DOK4; 9, INPP5E; 9, TEC; 9, SH3BP2; 12, CPSF1; 12, ASAP3;
12, TCEA3; 12, SNRPC; 12, LMO2; 12, CDC73; 12, POLR2C; 12, RNF40; 12, RARA; 14, ZNF365;
14, AFF3; 14, PTPN2; 14, BACH2; 14, UBASH3A; 14, IL12A; 14, TYK2; 14, IL37;

TABLE 15D
AsA GWAS validation clusters (FIG. 12). Listed by: MCODE cluster, gene;

1, ZYG11A; 1, ZYG11B; 1, SERP2; 1, SPCS3; 1, RNF5; 1, RNF169; 1, RNF11; 1, USP25; 1,
ZFAND5; 1, UBQLNL; 1, PPP1R9A; 1, SPCS2; 1, PARP11; 1, NPAS3; 1, SLIT2; 1, KCNQ5; 1,
KCNG3; 1, RAD23B; 1, GFM1; 1, SRP54; 1, FRRS1; 1, USP47; 1, FAM168A; 1, EPM2A; 1,
MRPL13; 1, WNT3A; 1, HBS1L; 1, DCAF12; 1, UROD; 1, DCAF6; 1, RPS27L; 1, RSRC1; 1,
SKIV2L; 1, RPL9; 1, RPL6; 1, SSR1; 1, DCUN1D3; 1, PPP2CA; 1, RPS10; 1, RPS25; 1, RPS18; 1,
RPL3; 1, RPL10A; 1, RPS6; 1, RPS5; 1, SEC61A1; 1, ARCN1; 1, CERKL; 1, TCEB1; 1, GAN; 1,
ASB14; 1, ANAPC7; 1, HERC1; 1, UBE2G1; 1, CUL5; 1, HECTD3; 1, FBXL22; 1, FBXW5; 1,
RNF25; 1, KCTD7; 1, SKP2; 1, CBLB; 1, UBE2R2; 1, HACE1; 1, RNF114; 1, SKP1; 1, ANAPC13;
1, RPL8; 1, UBA52; 1, PHC3; 1, RPS8; 2, NEUROD6; 2, KIAA1751; 2, MAPK9; 2, PAQR5; 2,
TUB; 2, OPN1SW; 2, MAPK13; 2, GZMA; 2, GPSM3; 2, NPY2R; 2, NPY1R; 2, GABBR1; 2,
NPY5R; 2, HTR5A; 2, SSTR2; 2, HTR1E; 2, CCR3; 2, CCR1; 2, OXER1; 2, CCR9; 2, CCR5; 2,
CXCR5; 2, PMCH; 2, CXCL16; 2, CXCR6; 2, CCL20; 2, OXGR1; 2, SLC12A2; 2, RAPGEF3; 2,
RAPGEF4; 2, NMUR2; 2, CX3CL1; 2, ADCY3; 2, GNB1; 2, GNG5; 2, PDE2A; 2, PDE7B; 2,
PDE8B; 2, GNB5; 3, INTS8; 3, GTF2E2; 3, GPN3; 3, RBM42; 3, EIF2B3; 3, EIF2AK2; 3, EEFSEC;
3, SLC30A9; 3, DAW1; 3, DIS3L2; 3, CIRH1A; 3, CWF19L2; 3, LSM2; 3, SRRM1; 3, SF3A3; 3,
DDX46; 3, CPSF2; 3, U2AF1L4; 3, SNRNP27; 3, SNRPA; 3, SNRPD3; 3, CD2BP2; 3, EIF2S1; 3,
ELF1; 3, SART3; 3, AMD1; 3, CCT8; 3, LSM8; 3, AGPAT1; 3, RPP21; 3, DPY30; 3, SUZ12; 3,
POLR2B; 3, NUDT2; 3, MOCS1; 3, NTPCR; 3, POLR2C; 3, SRPK1; 3, SRRM3; 3, PABPN1; 3,
ACIN1; 3, DHX15; 3, UTP15; 3, WDR36; 3, UTP6; 3, IMP4; 3, MPHOSPH10; 4, TCP11L1; 4,
MOB1B; 4, MOB1A; 4, LRRC39; 4, MLF1; 4, EML4; 4, MZT2B; 4, NDEL1; 4, KIF24; 4, NEDD1;
4, NEK2; 4, CEP97; 4, TUBB; 4, CEP63; 4, C2CD3; 4, NINL; 4, YWHAE; 4, HAUS4; 4, AHI1; 5,
PTPRK; 5, IL15; 5, TSLP; 5, IFNA21; 5, NR2F2; 5, PTPN18; 5, TAC3; 5, P2RY6; 5, NTS; 5,
KALRN; 5, EDNRB; 5, ERBB2IP; 5, CCL22; 5, CCL17; 5, PLCB4; 5, PLCB1; 5, SENP1; 5,
NFKBIL1; 5, PTAFR; 5, PPP2R3C; 5, OR5H15; 5, OR2J3; 5, OR5V1; 5, OR11A1; 5, OR2H1; 5,
OR2L13; 5, OR2M5; 5, OR5AC2; 5, OR5H2; 5, OR5K4; 5, OR10A7; 5, XCR1; 5, P2RY2; 5,
OR2M4; 5, OR52A5; 5, SLC9A5; 5, IL17RD; 5, OR4K1; 5, OR2B6; 5, ENSG00000250151; 5,
WIPF3; 5, VPS29; 5, ITPR3; 5, AHNAK2; 5, MAPK14; 5, REPS1; 5, ARRB1; 5, ARRB2; 5, STAM;
5, HIP1; 5, SYT9; 5, DAB2; 5, IL7R; 5, EGFR; 5, ARPC3; 5, AGFG1; 5, PRKCB; 6, USP37; 6,
TAP2; 6, TAP1; 6, UBLCP1; 6, JAZF1; 6, SOD1; 6, TNKS; 6, PELP1; 6, STAMBP; 6, CDKL1; 6,
NAA10; 6, TUBAL3; 6, TUBA3E; 6, FKBP9; 6, RUVBL1; 6, TTLL3; 6, MARS; 6, COPRS; 6,
DDX39B; 6, CHTOP; 6, BCLAF1; 6, HSP90AB1; 6, PSMB11; 6, PSMB8; 6, PSMB9; 6, CCNE2; 6,
CCT6A; 6, AHCY; 6, NOTCH4; 6, CCND1; 6, JAK2; 6, PSMB5; 6, ABCB5; 7, TADA1; 7, TONSL;
7, USP3; 7, HIST1H2BE; 7, PARPBP; 7, HIST1H1B; 7, CBX5; 7, BRD2; 7, HIST1H4L; 7,
HIST1H4K; 7, CMC2; 7, BMI1; 7, DLD; 7, PDHX; 8, SYNGR1; 8, LAMP1; 8, FLNB; 8, LSAMP; 8,
CNTN5; 8, LYPD6B; 8, GP2; 8, LY6G6C; 8, NEGR1; 8, THY1; 8, CNTN4; 8, ITGA3; 8, CD80; 8,
CD226; 8, PRMT5; 8, FAM20A; 8, CALU; 8, LTBP1; 8, PDIA6; 8, EVA1A; 8, C4A; 8, ITGAL; 8,
RAB44; 8, SLC15A4; 8, HVCN1; 8, CD47; 8, CIAPIN1; 8, ATP6VID; 8, APOM; 8, FLNC; 8,
ANGPTL3; 8, SERPINC1; 8, CPB2; 8, ITGB2; 8, ITGB5; 8, ALDH8A1; 8, FGG; 8, A1BG; 9, SIK2;
9, PPP1R11; 9, PPP4R2; 9, SH2D4A; 9, PPP1R1A; 9, PPP1R13B; 9, URI1; 9, NFATC3; 9, XRCC3;
9, INO80E; 9, IKBKE; 9, IFI27; 9, RPAP3; 9, KDM4A; 9, KIF5C; 9, YEATS4; 9, FOXR1; 9,
RSAD2; 9, IFI44; 9, EIF2AK1; 9, CTSO; 9, PPP1R8; 9, CSNK1G3; 9, PAPOLB; 9, CNST; 9,
RAD9B; 9, RAD51B; 9, UIMC1; 9, PLK3; 9, NBN; 9, CENPN; 9, CENPQ; 9, CENPL; 9, YWHAQ;
9, CDCA7; 9, CLSPN; 9, CDC7; 9, TWIST2; 9, LAMA2; 9, TRIM6; 9, TRIM5; 9, TRIM22; 9, IRF5;
9, OAS2; 9, OAS3; 9, HLA-DQB2; 9, CD274; 9, ORC3; 9, CACNA2D3; 9, TADA3; 9, HIST3H2A;
9, TCF7; 9, KAT5; 9, ATXN1; 9, MARK3; 9, KSR1; 9, YWHAH; 9, TTK; 9, ARHGAP11B; 9,
CYTH3; 9, KLC1; 9, KIF26A; 9, KIFC1; 9, KIFC2; 9, KIF2B; 9, ARF4; 9, DCTN1; 9, TIAM1; 9,
CD44; 9, ANK3; 9, POLA2; 9, CHEK2; 9, MYO1A; 9, ADCY10; 9, PPP1CC; 9, MYL12A; 12,
COL28A1; 12, COL8A2; 12, PPIB; 12, P4HA1; 12, COL8A1; 12, COL11A2; 12, ADAMTS3; 13,
WWC1; 13, MPP5; 13, LRP1; 13, AGO4; 13, ETS1; 13, ERN1; 13, KIAA0391; 13, AGO3; 13,
NOTCH3; 13, AGO1; 13, H2AFX; 13, HIST1H2BL; 13, HIST1H2BK; 13, ATP2A2; 13, ATF6B; 13,
HSPA1A; 13, HSPA1B; 13, PPID; 14, NLGN1; 14, SHANK2; 14, NRXN3; 14, NCAM2; 14,
DLGAP3; 14, DLGAP1; 14, KCNJ2; 14, DLG2; 14, SORBS2; 14, GRIA4; 14, ERBB4; 14, PDE4D;
15, TDRD9; 15, TDRD5; 15, MAEL; 15, TDRD6; 15, DDX6; 17, ZSCAN16; 17, ZSCAN23; 17,
ZSCAN30; 17, ZBED9; 17, ZNF396; 17, ZKSCAN4; 17, ZKSCAN3; 17, ZSCAN12; 17, ZKSCAN8;
17, ZNF24; 17, PGBD1; 18, PFKM; 18, PDXK; 18, NEU1; 18, MGST3; 18, GBE1; 18, UGDH; 18,
ERO1LB; 18, PRDX1; 18, TNFAIP6; 18, ASAH1; 18, GUSB; 18, ARSG; 18, PRDX6; 18, GGH; 18,
HYAL4; 18, PYGB; 18, ARSB; 18, TXNDC5; 18, GNS; 18, GPX5; 18, GPX6; 21, TNIP1; 21, LTB;
21, TNF; 21, LTA; 21, TNFAIP3; 21, EPHX2; 21, MPV17; 21, PEX13; 21, CROT; 21, CDYL2; 21,
STAT1; 21, NOS2; 21, ASL; 21, TAB1; 21, PSMA6; 21, CALML6; 21, ACSL1; 21, SCP2; 21,
ABCD2; 22, TYW1; 22, ZBTB11; 22, GPR149; 22, EXOSC4; 22, TTC27; 22, NAF1; 22, CCDC86;
22, SURF6; 22, ZNRD1; 22, RPF1; 22, TFB2M; 22, RRP15; 22, RTCA; 22, ABT1; 23, VAC14; 23,
PI4K2B; 23, INPP4B; 23, PLCD4; 23, IMPA2; 23, PIKFYVE; 23, PIP4K2A; 23, PIK3C3; 23, ATG5;
24, MRPL55; 24, MRPL48; 24, DMXL2; 24, C14orf2; 24, C17orf80; 24, ATP6V0B; 24, MRPS21;
24, ATP6V1G2; 24, ATP6V1C2; 24, CCDC115; 24, COX6B1; 24, ATP5L; 24, ACADVL; 25, RGS1;
25, CTSB; 25, FGF19; 25, NTF3; 25, SLC7A7; 25, SLCO2B1; 25, EREG; 25, CORO1A; 25, PTPRC;
25, WDFY4; 25, IKZF1; 25, TLR7; 25, BLK; 25, AREG; 25, CSF1R; 25, ANGPT2; 25, PLD4; 25,
F13A1; 28, PLA2G15; 28, TNFSF10; 28, HTRA2; 28, GDPD3; 28, POU5F1; 28, DNAJC1; 28,
DNAJA3; 28, DAPK2; 28, FGF2; 28, TERT; 28, STAT6; 28, PGR; 28, IFNGR1; 28, CSNK2B; 28,
SMAD1; 28, RASA1; 28, BAG5; 28, BAG2; 28, RPS6KA5; 28, CREB3L4; 28, PLA2G7; 28, PTGS2;
28, PLA2G16; 28, ALOX15; 28, CYP4A22; 28, ALDH9A1; 28, CYP4A11; 28, UGT2B4; 28,
ALDH1A1; 28, AGER; 32, PMS1; 32, PMS2; 32, FAN1; 32, FANCC; 32, DCLRE1A; 33, FER; 33,
MGAT3; 33, CDH3; 33, KLK7; 33, FLOT1; 33, SKIL; 33, CTNNA3; 33, CDH18; 33, CDH4; 33,
CDH24; 33, PKP4; 33, MEF2A; 33, CDH1; 33, MAPK3; 34, VPS53; 34, RABGEF1; 34, RAB3C; 34,
DOC2A; 34, RAB6A; 34, RAB23; 34, C12orf4; 34, RAB17; 34, BOLA3;

TABLE 15E
E-MTAB-11191 DE clusters (FIG. 15). Listed by: MCODE cluster, gene;

1, TROVE2; 1, TMEM43; 1, RNF145; 1, TSPAN18; 1, MYCBP2; 1, LDLRAD4; 1, TXNIP; 1,
DISC1; 1, ZYG11B; 1, WIPI1; 1, HERC4; 1, KLHL9; 1, VPRBP; 1, CUL2; 1, CUL5; 1, FBXW2; 1,
FBXL16; 1, ITCH; 1, WWP1; 1, UBR2; 1, UBE2J1; 1, UBE2W; 1, FBXO11; 1, LONRF1; 1, DZIP3;
1, RNF130; 1, KLHL5; 1, KBTBD7; 1, KLHL2; 1, KCTD6; 1, MYLIP; 1, CFAP97; 1, UBE2G2; 1,
ZBTB16; 1, KLHL22; 1, SOCS1; 1, FBXO21; 1, ASB8; 1, KLHL3; 1, FBXO22; 1, DTX3L; 1,
RNF126; 1, UBE2L6; 1, KCTD7; 1, UBE2K; 1, HECW2; 1, RNF144B; 1, TRIM21; 1, FBXO30; 1,
UBE2C; 1, UBE2B; 1, ATG7; 1, SIAH1; 1, JAG1; 1, SMURF2; 1, TTN; 1, UBE2D1; 1, UBE2D3; 1,
FBXW7; 1, FBXO6; 1, TRIM32; 1, ANAPC1; 1, ANAPC5; 2, ZBED6; 2, TMED4; 2, SPCS3; 2,
PAIP2; 2, TIMM10B; 2, MAP4; 2, SAT1; 2, UHMK1; 2, ZNF207; 2, HNRNPUL2; 2, HN1L; 2,
NUDT3; 2, SREK1; 2, FNBP4; 2, FASTKD2; 2, FAM120A; 2, ZCCHC6; 2, STK16; 2, DPH3; 2,
DNAJA2; 2, SMG7; 2, UPF1; 2, SFSWAP; 2, ERLIN1; 2, DNAJC7; 2, SNX1; 2, CSDE1; 2, EIF3L;
2, EIF3K; 2, COA1; 2, YTHDC1; 2, LUC7L; 2, EIF4B; 2, CD164; 2, RPS5; 2, RPS19; 2, RPLP0; 2,
NACA; 2, RPL37A; 2, RPL10; 2, DOCK4; 2, GSPT1; 2, RPL23A; 2, RPL14; 2, SIL1; 2, EIF1AX; 2,
HABP4; 2, RPS4X; 2, RPL7A; 2, RPL3; 2, EIF5A; 2, RPLP2; 2, RPL38; 2, RPL10L; 2, EIF5B; 2,
RPS11; 2, C18orf32; 2, CCDC130; 2, KIAA1143; 2, NKTR; 2, LUC7L2; 2, RPS9; 2, ATXN2; 2,
ATXN1; 2, SMG1; 2, GART; 2, PRPF8; 2, SNRNP200; 2, SNRPA1; 2, SYF2; 2, SLU7; 2, SF3B3; 2,
SRRM2; 2, SNRPN; 2, SRRM1; 2, MAGOH; 2, PPIH; 2, HNRNPA1; 2, HNRNPA3; 2, DDX46; 2,
SRRT; 2, ISY1; 2, SRSF6; 2, U2SURP; 2, HNRNPU; 2, SRSF11; 2, CWC25; 2, HNRNPUL1; 2,
PTBP1; 2, RBMX; 2, U2AF1L4; 2, HNRNPH1; 2, WBP4; 2, HNRNPH2; 2, EEF2; 2, AQR; 2,
RPS20; 2, RPL18; 2, RPS15; 2, RBM17; 2, PABPC1; 2, EIF3H; 2, AMD1; 2, SF1; 2, PPP2R2A; 2,
CLK4; 2, EIF3M; 2, UBQLN4; 2, EEF1G; 2, BUD31; 2, GNB2L1; 2, STRBP; 2, PCBP2; 2, NARS;
2, EIF3B; 3, PILRA; 3, ICAM2; 3, KCNA3; 3, SAMSN1; 3, RB1CC1; 3, FPR2; 3, TMC6; 3, CD177;
3, SCAMP1; 3, LAMTOR3; 3, ATP11A; 3, TMBIM1; 3, TSPAN14; 3, CD59; 3, MOSPD2; 3,
HGSNAT; 3, KCNAB2; 3, CKAP4; 3, CD47; 3, DNAJC5; 3, ITGAL; 3, FCAR; 3, PLAUR; 3,
GPR97; 3, MCEMP1; 3, RAP2B; 3, C3AR1; 3, CLEC4D; 3, CD53; 3, SLC15A4; 3, BST1; 3,
CEACAM8; 3, TMEM30A; 3, PGRMC1; 3, CMTM6; 3, CEACAM1; 3, SLC2A3; 3, ADAM8; 4,
SLC11A2; 4, TTLL5; 4, NIN; 4, MOB1A; 4, MACF1; 4, TMEM214; 4, RGS2; 4, KCNJ15; 4,
KCNJ2; 4, SFXN3; 4, SERPINB1; 4, DEFA1; 4, WDR42A; 4, CRYBG3; 4, PER1; 4, TDRD7; 4,
SLAIN2; 4, SIRPG; 4, SKAP2; 4, CCDC18; 4, BBS2; 4, BBS1; 4, AUP1; 4, ATXN3; 4, ATP2B1; 4,
ELANE; 4, LCN2; 4, NEU1; 4, DEFA4; 4, KPNB1; 4, BPI; 4, ERP44; 4, ARG1; 4, ATG4B; 4,
S100A10; 4, ANKRD32; 4, TCTN3; 4, RAB3IP; 4, TMEM216; 4, HAUS5; 4, CKAP5; 4, ODF2; 4,
SFI1; 4, CDK1; 4, KIF24; 4, MAPRE1; 4, DYNC1H1; 4, OFD1; 4, CEP97; 4, TUBB; 4, SCLT1; 4,
TTBK2; 4, RAB11A; 4, DCTN3; 4, TUBA4A; 4, B9D2; 4, HAUS4; 4, ALMS1; 4, SPTAN1; 4,
CANT1; 4, LTF; 4, MMP8; 4, GGH; 4, TIMP2; 4, ORM1; 4, QSOX1; 4, CAMP; 4, PTX3; 4, QPCT;
4, CRISP3; 4, OLFM4; 4, CSNK1E; 4, CXCR4; 4, FPR1; 4, GABBR1; 4, GPR183; 4, CCL5; 4,
GNAI3; 4, CXCR3; 4, S1PR2; 4, ANXA1; 4, GPSM2; 4, VSIG4; 4, CCL28; 4, GPR18; 4, LPAR5; 4,
GNAI2; 4, CXCL16; 4, CXCR5; 4, ADORA3; 4, GNB4; 4, MGAM; 4, SIRPA; 4, RAP2C; 4,
CYSTM1; 4, SLC11A1; 4, VCL; 4, YWHAE; 5, NENF; 5, ST8SIA4; 5, PHACTR4; 5, NETO2; 5,
RNASEL; 5, IP6K2; 5, NCAM1; 5, FAM98B; 5, DNAJC16; 5, SMCHD1; 5, STAT2; 5, GOLGA3; 5,
RSAD2; 5, IFIT3; 5, IFIT1; 5, MX2; 5, IFIT2; 5, TRIM6; 5, TRIM22; 5, OAS2; 5, TRIM2; 5,
TRIM46; 5, OAS3; 5, CCZ1B; 5, TRIM25; 5, GBP1; 5, HLA-DMA; 5, HLA-DMB; 5, NR3C2; 5,
CSPP1; 5, TAP1; 5, HLA-C; 5, HLA-G; 5, HLA-E; 5, HLA-DRB1; 5, HLA-DQB1; 5, HLA-DRB5; 5,
HLA-DRA; 5, HLA-DPA1; 5, OASL; 5, BICD1; 5, OAZ2; 5, KIF5B; 5, AZIN1; 5, RAB7A; 5,
MTPN; 5, SPTBN1; 5, DCTN4; 5, CAPZA2; 5, COG8; 5, COG7; 5, ANK3; 5, COPZ1; 5, COPA; 6,
OGFOD1; 6, NEK7; 6, ISG20; 6, ZNHIT6; 6, IKBKAP; 6, HSPA6; 6, PUS1; 6, HMBOX1; 6,
NSUN7; 6, HNRNPLL; 6, DYNLT1; 6, DNAH1; 6, DDX52; 6, DIEXF; 6, NGDN; 6, LTV1; 6,
DKC1; 6, NOP2; 6, RRP1B; 6, NSUN6; 6, NSUN4; 6, DDX59; 6, KRI1; 6, NOL6; 6, PPAN; 6,
GLTSCR2; 6, RBM28; 6, SP100; 6, CADM1; 6, RRP15; 6, UTP14A; 6, SLC25A6; 6, DDX39B; 6,
NUDC; 6, NDEL1; 6, CLIP1; 6, CLASP2; 6, LSM12; 6, TAOK1; 6, PADI2; 6, MAN2B1; 6, DSN1;
6, GCA; 6, C6orf120; 6, TRAPPC1; 6, STK11IP; 6, SDCBP; 6, AZU1; 6, API5; 6, MUTYH; 6,
FRAT1; 6, FRAT2; 6, MAPRE2; 6, RAB1A; 6, CD55; 6, GOSR2; 6, PSMC2; 6, SHFM1; 6,
PPP2R5E; 6, PPP2R5A; 6, HRAS; 6, KDM7A; 6, APBB1IP; 6, MPRIP; 6, QKI; 6, ARAF; 6, RAF1;
6, ZC3HAV1; 6, AGTRAP; 6, AP3B1; 6, POM121C; 6, NUP160; 6, NUP210; 6, NUP214; 6,
NUP205; 6, NUP43; 6, NUP133; 6, CECR1; 6, PRKCD; 6, SMC1A; 6, RAD21; 6, NAT10; 6,
ARRB1; 7, TP53INP1; 7, USP24; 7, ZNF385A; 7, ZBTB4; 7, RAB32; 7, RAB31; 7, PRDM1; 7,
ZRSR2; 7, RC3H2; 7, IPO9; 7, PLSCR1; 7, SREBF2; 7, STAT4; 7, RAB21; 7, HINT3; 7, STK4; 7,
RAB13; 7, ELK4; 7, ELK3; 7, MKNK1; 7, DUSP16; 7, YIPF4; 7, HSPA1L; 7, DDX60L; 7, IFI44; 7,
PARP9; 7, SP110; 7, DACH1; 7, RALB; 7, NRF1; 7, PPP6R3; 7, NR1D2; 7, CSGALNACT2; 7,
CSGALNACT1; 7, TP53RK; 7, CRIP1; 7, RAB30; 7, RAB36; 7, IFIT5; 7, CMPK2; 7, SRPK1; 7,
KTN1; 7, SHISA5; 7, IPO7; 7, CHST15; 7, VCAN; 7, CHST11; 7, ZBED1; 7, CEBPD; 7, EBF1; 7,
TRIB1; 7, MED19; 7, MED14; 7, MED26; 7, CDK10; 7, IL21R; 7, LMAN2L; 7, LMAN2; 7,
TRAPPC10; 7, SEC22C; 7, STX17; 7, IGFBP3; 7, MAP4K1; 7, MAPKAPK2; 7, DNTTIP1; 7,
DDX58; 7, SAMD9L; 7, PRKCSH; 7, MBTPS1; 7, RCN1; 7, SBNO1; 7, CHD3; 7, MED13; 7,
SMYD2; 7, BLZF1; 7, EHBP1L1; 7, THRAP3; 7, RBM14; 7, BCLAF1; 7, PAG1; 7, CBX5; 7,
NCOR1; 7, NCOR2; 7, BACH2; 7, KIF5C; 7, BBX; 7, PHF21A; 7, RAB33B; 7, ATG16L1; 7,
GMFB; 7, SCRN1; 7, ING2; 7, SAP30; 7, DAAM1; 7, RAB2A; 7, RAB22A; 7, F5; 7, LRP1; 7, LSR;
7, APOC3; 7, KHSRP; 7, MEN1; 7, CSNK2A1; 7, APC; 7, CD3E; 7, APPL2; 7, TFG; 7, LGALS1; 7,
ANXA11; 7, NFKBIA; 7, CEBPB; 7, SIN3B; 7, CBX6; 7, PHC1; 7, RING1; 7, PHC2; 7, MAPK14;
7, NCOA3; 7, AGFG2; 7, SEC31A; 7, SEC24D; 7, EIF2AK2; 7, TGFA; 7, HELZ2; 7, NCOA2; 7,
LPL; 7, MED1; 7, TP53; 7, ABCB1; 7, FCHO2; 7, AGFG1; 7, FNBP1; 7, NUMB; 7, REPS2; 7,
AMPH; 7, BIN1; 7, ARPC5; 7, RAB5A; 7, STAM2; 7, PACSIN2; 7, CD4; 7, AAK1; 9, PLEKHA1;
9, PGM3; 9, TNKS2; 9, TET2; 9, PIH1D1; 9, TMEM55A; 9, NUCKS1; 9, SKIL; 9, KLF6; 9,
RNF146; 9, TRAP1; 9, CR1; 9, HEXIM1; 9, PHAX; 9, RPRD2; 9, TAF8; 9, INTS4; 9, NABP1; 9,
SNAPC2; 9, CCNI; 9, CDK14; 9, CCNY; 9, CBFA2T2; 9, C11orf58; 9, SETD5; 9, SRBD1; 9,
POU2F2; 9, BATF2; 9, USP13; 9, SUPT4H1; 9, EPAS1; 9, ANKRD22; 9, SH3BP2; 9, CDC14A; 9,
EGLN1; 9, KMT2A; 9, CDKN2D; 9, CDK4; 9, KDM6B; 9, PTEN; 9, E2F3; 9, CDK6; 9, CCND2; 9,
CCND3; 9, RUNX1; 9, LIMD1; 9, EIF4E3; 9, RC3H1; 9, AGO2; 9, CCNT1; 9, ELL2; 9, AFF1; 9,
SMAD3; 10, ZMIZ1; 10, RFFL; 10, TBXAS1; 10, PTGDS; 10, TLR5; 10, USP5; 10, SLC8A1; 10,
LRRFIP2; 10, HLX; 10, KAT7; 10, FLT3; 10, WDR11; 10, PPP1R13B; 10, PEA15; 10, VPS72; 10,
DDX3X; 10, HIPK3; 10, CTBP2; 10, CYLD; 10, IRS2; 10, IKBKE; 10, LY96; 10, GAB2; 10,
CSRP2BP; 10, DR1; 10, MSL1; 10, MSL3; 10, CFLAR; 10, TNFSF10; 10, TNFRSF10A; 10, TLR1;
10, CASP10; 10, ORAI2; 10, FAS; 10, RASGRP2; 10, EP400; 10, TLR4; 10, BCL10; 10, WDFY3;
10, PIP4K2B; 10, ARL6IP4; 10, FADD; 10, TCF7L2; 10, TCF7; 10, BCL9L; 10, TLE4; 10, TLE2;
10, GRAP; 10, TANK; 10, APBA3; 10, APOA1BP; 10, SHC1; 10, PTGS2; 10, PTGS1; 10, CYP2U1;
10, PLA2G6; 10, KBTBD2; 10, CYP4F2; 10, CYP4F3; 10, ALOX15B; 10, PIK3R5; 10, PIK3CG; 10,
ITPR3; 10, ITPR1; 10, PRKCA; 10, XIAP; 10, INHBB; 10, ACVR1C; 10, ACVR1B; 11, SRPR; 11,
USP32; 11, USP15; 11, POLR3B; 11, WDR83OS; 11, MRPL9; 11, GUF1; 11, MRPL19; 11,
ATPAF2; 11, PEBP1; 11, MRPS16; 11, AARS2; 12, USP28; 12, ZMPSTE24; 12, GATAD2B; 12,
PRRC2C; 12, PRRC2B; 12, PPARD; 12, ZBTB7A; 12, PABPC3; 12, DYRK2; 12, KDM1A; 12,
RCOR1; 12, YWHAH; 12, ATM; 12, MAOA; 12, PSMC3IP; 12, IKBKG; 12, MDM4; 12, ETS2; 12,
EDC4; 12, TNRC6A; 12, TNRC6C; 12, TNRC6B; 12, XPO5; 12, AGO3; 12, DCP2; 12, AGO4; 12,
NLK; 12, TSEN54; 12, MAML3; 15, RTN3; 15, OSBPL7; 15, OSBPL3; 15, OSBPL2; 15, PPM1L;
15, HP1BP3; 15, TSPAN2; 15, RNF13; 15, OSBPL1A; 15, OSBPL10; 15, LAMP2; 15, MAGT1; 15,
VAPA; 15, VNN1; 15, CD63; 15, BRI3; 15, NDUFC2; 15, MANBA; 15, CEACAM6; 15, ATP11B;
15, CANX; 15, PSEN1; 15, SIN3A; 15, TRAF6; 15, VAMP2; 16, NDUFA3; 16, NDUFB3; 16, MT-
ND2; 16, MRPL42; 16, MRPL41; 16, MRPL52; 16, MRPL30; 16, NDUFB4; 16, MT-ND3; 16,
HTATIP2; 16, FAM3C; 16, MRPL44; 16, UXT; 16, TSFM; 16, DNAJC30; 16, NDUFS1; 16,
NDUFB11; 16, PTCD3; 16, NDUFB8; 16, NDUFB10; 16, APOOL; 16, OXA1L; 18, PANK2; 18,
LRRC4; 18, IL18RAP; 18, LILRA6; 18, LILRA2; 18, LILRB3; 18, IKZF3; 18, TRAF3IP3; 18, SELL;
18, LY9; 18, CD52; 18, VNN3; 18, NTNG2; 18, CPM; 18, CD79A; 18, ARHGAP5; 18, GNA13; 18,
S100A9; 18, VNN2; 18, FCGR3B; 18, CEACAM4; 18, AQP9; 18, LTB4R; 18, F2RL1; 18, P2RY10;
18, CD28; 18, TRIO; 18, ABLIM1; 19, TBC1D20; 19, PHF19; 19, PHF1; 19, PKNOX1; 19,
KANSL1; 19, IFFO2; 19, NFYA; 19, ZFAND6; 19, HMGB3; 19, MXD1; 19, STRA13; 19, DUSP1;
19, PSIP1; 19, TDP1; 19, SPIN2B; 19, FANCI; 19, MAX; 19, TAF9B; 19, TAF2; 19, TAF7; 19,
DSTN; 19, NAP1L1; 19, CHD1L; 19, CHD6; 19, DIDO1; 19, MLLT3; 19, BCOR; 19, BAZ1A; 19,
CCDC101; 19, TAF12; 19, ATXN7L1; 19, HBP1; 19, USP9X; 19, XRCC4; 19, RNF168; 19, UIMC1;
19, RAD1; 19, TOP3A; 19, DCLRE1C; 19, RMI1; 19, SPIDR; 19, NELFE; 19, TCEB3; 19, MSH3;
19, PIAS4; 19, HUS1; 19, SUPT16H; 19, KDM4B; 19, ZC3H13; 19, ASH1L; 19, ARID4B; 19,
ARID4A; 19, ARFIP1; 19, TRRAP; 19, TCEA1; 19, UVSSA; 19, RSF1; 19, EHMT1; 19, RBBP4; 19,
TBK1; 19, CHD9; 19, NCOA6; 19, PEX3; 19, PEX16; 19, PPARA; 19, ACSL1; 19, PEX13; 19,
ACSL5; 19, CROT; 19, GSTK1; 19, PECR; 19, GCDH; 19, ECH1; 19, MRE11A; 19, NBN; 19,
MCCC2; 19, ACAT2; 19, ACOX1; 19, ACADVL; 19, FARSB; 20, IL13RA1; 20, TOR1A; 20,
TESPA1; 20, TOR1B; 20, LAT; 20, STAT1; 20, RAD23B; 20, TULP4; 20, SOCS5; 20, COPS6; 20,
GTF2H2; 20, CDK7; 20, PSMB2; 20, PSMB7; 20, PSMC4; 20, PSMD4; 20, JAK2; 20, MAPK1; 20,
PSMD6; 21, STXBP5; 21, RABEP2; 21, SLC4A7; 21, STX3; 21, STX11; 21, STX7; 21, EHD4; 21,
RHOBTB3; 21, ZMYM2; 21, LRRFIP1; 21, FGFR1OP2; 21, KIF27; 21, NBAS; 21, KIF1B; 21,
VPS53; 21, VPS52; 21, MAN1A1; 21, MAN1A2; 21, CUX1; 21, MAN1C1; 21, MAN2A2; 21,
VPS51; 21, MAN2A1; 21, KIFC2; 21, TMF1; 21, CCDC186; 21, GANAB; 21, NSF; 23, YLPM1; 23,
NAIP; 23, MTHFR; 23, IL12RB1; 23, MYO1G; 23, MCMBP; 23, SENP1; 23, ZNFX1; 23, MIER1;
23, IFRD1; 23, KPNA6; 23, ZNF76; 23, SCYL2; 23, GGTLC1; 23, GSS; 23, GSTA1; 23, MGST3;
23, GSTM3; 23, GATAD1; 23, REPIN1; 23, IL6ST; 23, MAF; 23, JDP2; 23, FOSL2; 23, PGD; 23,
EPHA4; 23, UBN1; 23, HIRA; 23, FKBP2; 23, GPX4; 23, RERE; 23, MGA; 23, DOCK8; 23,
DNPEP; 23, FBRSL1; 23, GSR; 23, SOCS7; 23, ZNF44; 23, STARD10; 23, IL23A; 23, CSF2RA; 23,
GGT7; 23, CSAD; 23, NEDD9; 23, FGFR2; 23, JMJD6; 23, DOCK5; 23, DOK2; 23, KIDINS220; 23,
SOS2; 23, CREM; 23, INO80D; 23, CIR1; 23, CLOCK; 23, ZNF827; 23, ZNF219; 23, SIRT2; 23,
FUBP3; 23, UBTF; 23, GATA2; 23, GATA3; 23, RRN3; 23, TYMS; 23, KIAA0101; 23, IKZF1; 23,
ITGB7; 23, RBL2; 23, NMI; 23, H2AFY; 23, H2AFY2; 23, CAST; 23, EMC1; 23, KPNA4; 23,
RRM2; 23, MARCKS; 23, SHMT2; 23, IL2RG; 23, PCMT1; 23, UBR5; 23, HELLS; 23, HFE; 23,
RAI1; 23, KLHDC10; 23, TCF12; 23, STAT5A; 23, BMX; 23, TCF4; 23, BRD9; 23, CD19; 23,
CARD16; 23, BCL11B; 23, UBXN4; 23, BCL11A; 23, TAF1C; 23, BAZ1B; 23, EMD; 23, RYBP;
23, AUTS2; 23, MAP1LC3B; 23, ASXL2; 23, UBAP2L; 23, ASXL1; 23, GAB1; 23, PHF20L1; 23,
PBRM1; 23, WAC; 23, ARID2; 23, SMARCD1; 23, BCL7A; 23, BCL7C; 23, SMARCC1; 23,
ARID1B; 23, PGM2L1; 23, CSNK2B; 23, USP7; 23, GNPTAB; 23, EPHB1; 23, APH1B; 23,
HMGB2; 23, APEX1; 23, TRIM33; 23, EHMT2; 23, TKT; 23, ENO1; 23, ENO2; 23, ENO3; 23,
PGM2; 23, G6PC3; 23, DBT; 23, TPI1; 23, PRKCZ; 23, NFE2L2; 23, FOS; 23, CASP1; 23, CASP5;
23, NLRC4; 23, IFI16; 23, CASP4; 23, NLRP6; 23, AIM2; 23, HDAC1; 23, MDM2; 23, EIF4E2; 23,
C1QB; 23, NFIC; 23, PRKCB; 23, ADAM17; 23, KLF5; 23, YY1; 23, ACSL3; 23, NRIP1; 23,
SUN2; 23, SUN1; 23, LMNB1; 23, ACD; 23, ALDH6A1; 23, DLST; 23, TTC39C; 23, CYFIP2; 23,
CRK; 23, VAV3; 23, RAPH1; 23, ZNF511; 23, NHSL2; 23, UBXN11; 23, NCKAP1L; 23, ELMO2;
23, ABI2; 25, GPBP1L1; 25, MSI2; 25, CNOT8; 25, TOB1; 25, RQCD1; 25, CPEB2; 25, CPEB3; 25,
CPEB4; 25, CNOT2; 25, CNOT6L; 25, MYB; 25, RAD54L2; 25, PDE12; 25, PUM1; 25, DDX6; 25,
CNOT7; 28, NUDT16; 28, POLR2J3; 28, UPP1; 28, DCTPP1; 28, IFNAR1; 28, IFNGR1; 28,
IFNGR2; 28, CAMK2D; 28, CACNA2D4; 28, RASGRF2; 28, NAMPT; 28, MEF2A; 28, TTC5; 28,
LRP10; 28, PARP1; 28, CREB3L2; 28, ENTPD1; 28, NT5C2; 28, PDE4A; 28, PDE8A; 28, PDE4B;
28, NT5C3A; 28, AHCYL2; 28, AHCYL1; 28, ADK; 28, CALM2; 29, TMC8; 29, MAP3K12; 29,
TNIK; 29, MINK1; 29, MAP3K4; 29, MAPK8IP3; 29, NFATC2; 29, NSMAF; 29, ZBP1; 29,
MAP3K3; 29, RASAL3; 29, CD40; 29, MS4A1; 29, TDP2; 29, CRADD; 29, TRAF1; 29, TRADD;
29, CASP2; 29, PTBP2; 29, MAP2K6; 29, SPAG9; 29, BNIP2; 29, PELI1; 29, PELI2; 29, TAB1; 29,
TAOK3; 29, GADD45A; 29, MAP2K4; 29, CHUK; 29, MAP3K8; 29, LRRK2; 31, KDM5A; 31,
KDM5C; 31, EZR; 31, DMXL1; 31, MT-ATP6; 31, ATP6V1F; 31, ATP6V1C1; 31, KIAA2013; 31,
ATP6V1H; 31, PPA2; 31, ATP6V0E1; 31, ATP6V1E1; 31, ATP1B1; 33, KPNA1; 33, THUMPD1;
33, PRMT2; 33, EIF2D; 33, FCF1; 33, NOL9; 33, EXOSC10; 33, TRMT1L; 33, DDX50; 33, RTCA;
33, CIRH1A; 33, NAA25; 33, SDAD1; 33, MRPL43; 33, POLR1C; 33, BANF1; 33, G3BP1; 33,
PRMT5; 33, PABPC4; 33, FBL; 33, APRT; 33, PRKRA; 38, MTMR3; 38, MTMR6; 38, MTMR2; 38,
MTMR14; 38, IL1RAP; 38, MPI; 38, PFKFB3; 38, PFKFB4; 38, LRIG1; 38, PREX1; 38, GRB10; 38,
FLT3LG; 38, LAT2; 38, FCER1A; 38, F11R; 38, EML4; 38, RANBP9; 38, FZD3; 38, CTNND1; 38,
FGFR1; 38, FLT1; 38, DOCK7; 38, IL1R1; 38, CD79B; 38, HGF; 38, TRAT1; 38, TIAM1; 38, HK2;
38, PIK3R1; 38, DENND1B; 38, FKBP5; 38, FOXO1; 38, THEM4; 38, PFKFB2; 38, RICTOR; 38,
AKT3; 38, CDC37; 38, RALGAPA2; 38, AKT2; 38, CREB1; 38, BCL6; 56, PPAT; 56, GLUD1; 56,
HAL; 56, CARNS1; 56, EEF2K; 56, GLS; 56, C14orf159; 56, IDH3G; 56, CS;

TABLE 15F
Random clusters (FIG. 12). Listed by: MCODE cluster, gene;

1, SMO; 1, MAP2K6; 1, RGS11; 1, AKAP13; 1, GNAI3; 1, ADCY9; 1, APLNR; 1, SSTR2; 1, FPR3;
1, CASR; 1, LPAR3; 1, RXFP3; 1, NPY4R; 1, GPR55; 1, TAS2R20; 1, CXCL11; 1, CCL19; 1,
P2RY14; 1, CAMK4; 1, NMU; 1, CCR7; 1, HEBP1; 1, ADCY8; 2, GLP2R; 2, VIPR1; 2, TAAR1; 2,
SCT; 2, SCTR; 2, CALCA; 2, VIP; 2, TAAR5; 2, GPHB5; 2, TSHR; 3, MRPL19; 3, MRPL44; 3,
MRPL39; 3, HIBCH; 3, RPL23L; 3, MRPS35; 3, MRPS18B; 3, MRPS36; 3, MRPL57; 3, MRPS27;
3, MRPL42; 4, TMCO2; 4, NSA2; 4, SRRM2; 4, UTP23; 4, USP39; 4, CASP6; 4, EPPK1; 4, SF3B5;
4, MRPS10; 4, SNRPA; 4, NHP2L1; 4, CDC5L; 4, MRPL13; 4, PABPN1L; 4, MRPS12; 4, DEPDC7;
4, GCFC2; 4, SART1; 4, MSI1; 4, DNAJC4; 4, RALY; 4, PTBP1; 4, FUBP1; 4, SNRNP40; 4, SF3B4;
4, RP9; 4, CAPG; 4, HNRNPLL; 4, ZNHIT3; 4, EIF4G3; 4, TFIP11; 4, SLC7A9; 4, SNRNP27; 4,
CCDC97; 4, CSTF2T; 4, ZMAT5; 4, WDR33; 4, HSPA12A; 4, WDR83OS; 4, SNRNP200; 4,
PLRG1; 4, DDX17; 4, PPIH; 4, AQR; 4, GEMIN4; 5, UBOX5; 5, TRAF7; 5, KLHL25; 5, ENC1; 5,
FBXL5; 5, MGRN1; 5, FBXO15; 5, KCTD7; 5, MYLIP; 5, RNF130; 5, FBXW9; 5, MIB2; 5, UBR2;
5, CIAO1; 5, UBA5; 5, FMN1; 5, UBE2L3; 7, GNA11; 7, CCL15; 7, P2RY6; 7, PTAFR; 7, P2RY1;
7, HCRTR2; 7, PTGER1; 7, XCR1; 7, CYSLTR2; 7, XCL1; 7, P2RY11; 8, MUCL1; 8, GALNT11; 8,
GALNT15; 8, MUC4; 8, MUC12; 8, ST3GAL3; 8, GCNT1; 8, MUC5B; 8, ST3GAL3; 8, MUC12; 8,
GCNT1; 8, MUCL1; 8, GALNT11; 8, MUC4; 8, GALNT15; 8, MUC5B; 9, SCARB2; 9, ARPC1A; 9,
CD3D; 9, AP2M1; 9, PSCA; 9, CD52; 9, GZMK; 9, ALPL; 9, DCAF4; 9, ZDHHC17; 9, CD48; 9,
FEM1C; 9, HCST; 9, COPS8; 9, LYPD5; 9, STON2; 9, CEACAM5; 9, EGF; 9, SPRY2; 9, IPMK; 9,
ACTR3C; 9, CD3G; 9, DCAF5; 9, PTPN18; 9, SYNJ2; 9, ALPPL2; 9, PRSS21; 9, SYT11; 9, CD1D;
9, COPS2; 9, DCUN1D1; 9, MSLN; 9, AP4M1; 9, ARPC3; 9, CTLA4; 9, FEM1B; 9, CD1A; 9,
PICALM; 9, SPRN; 9, RECK; 9, LSAMP; 10, RAB7B; 10, FCGR1B; 10, COX18; 10, SPTB; 10,
YKT6; 10, LGMN; 10, RAB3GAP2; 10, GSPT1; 10, COPB2; 10, MAPRE3; 10, CHTOP; 10,
CYTH4; 10, KIF7; 10, TMED10; 10, SCFD1; 10, TUBB6; 10, CDC20B; 10, KIF13B; 10, KIF11; 10,
GBP7; 10, ARFIP2; 10, VCAM1; 10, HLA-DMB; 10, TUBGCP6; 10, HLA-DRB5; 10, SEC61A1;
10, COPG2; 10, RPS6KC1; 10, KIFAP3; 10, MAN2A2; 10, KIFC2; 10, GUF1; 10, EARS2; 10,
DCTN4; 10, RPLP2; 10, DYNC1I1; 10, PDS5B; 10, N6AMT1; 10, OAS3; 10, PLSCR1; 10, IRF6; 10,
RAB6A; 10, ARF5; 10, RPL12; 10, TMEM115; 10, CENPE; 10, TRIM46; 10, ARHGEF5; 10,
TUBA3C; 10, MAN1A1; 10, C4orf33; 11, RRP7A; 11, GRWD1; 11, ZNHIT6; 11, ACIN1; 11,
WDR74; 11, ATAD3C; 11, FAM86C1; 11, RPF1; 11, NMD3; 11, TYW1; 11, NSUN3; 11, DPH5; 11,
DPH2; 11, RIOK1; 11, NSUN2; 11, TMEM8A; 11, GNL1; 11, TRUB2; 11, DDX24; 11, RANBP6;
13, AGPAT5; 13, AGPAT6; 13, PPAPDC1B; 13, GPD1; 13, LPIN3; 13, PNLIP; 13, GPAM; 13,
MOGAT2; 16, IL12RB2; 16, PDGFB; 16, FGG; 16, AGGF1; 16, FAM20C; 16, STAT3; 16,
SERPINE1; 16, KTN1; 16, TNS3; 16, STAT1; 16, DOK1; 16, CTNND1; 16, TOR4A; 16, IGF2; 16,
HGF; 16, SLC9A3R2; 16, TNS4; 16, KIAA1549; 16, KIT; 16, KLK3; 16, PEBP1; 16, MMRN1; 16,
TMEM30A; 16, LPGAT1; 16, CPNE3; 16, LAMTOR1; 16, AGPAT2; 16, TMC6; 16, TMEM179B;
16, LPCAT1; 16, CD63; 16, PSEN1; 16, TRPM2; 16, NETO2; 19, ATP6V0A4; 19, ATP6AP1L; 19,
ATP6V1F; 19, NDUFA6; 19, NDUFS8; 19, C14orf2; 19, KDM5A; 19, KDM5B; 19, NDUFA9; 19,
ATP6V1E1; 19, ATP5O; 19, ATP1A2; 20, PEX1; 20, MPV17; 20, CHD8; 20, ZNF143; 20, PHYH;
20, IDE; 20, AMACR; 20, DAO; 20, PEX14; 20, HYLS1; 20, MUL1; 20, PAF1; 20, PXMP4; 20,
PEX2; 20, RNF5; 20, TULP2; 20, RNF20; 20, SUPT5H; 20, WAC; 20, INTS7; 20, CTR9; 20,
SUPT6H; 20, TCEB3CL2; 21, HSD3B2; 21, HSD3B1; 21, CYP11A1; 21, SRD5A3; 21, UGT1A7;
21, HSD11B1; 21, SRD5A1; 27, E2F8; 27, CENPU; 27, SGOL2; 27, ASF1B; 27, ARL2; 27, TRIB3;
27, DIAPH3; 27, SMC4; 27, MYBL2; 27, KIAA1524; 27, TOP2A; 27, CIT; 27, TROAP; 27,
RECQL4; 27, FANCD2; 27, HIST1H4C; 27, ANTXR1; 27, HAT1; 27, NUP153; 27, CEP70; 27,
TPX2; 27, ANTXRL; 27, HIST1H41; 27, CDC25A; 28, KLF5; 28, PTX3; 28, SEMG1; 28, DDX58;
28, RAC1; 28, UNC13D; 28, IFNA16; 28, ERP44; 28, LTF; 28, IFNA10; 28, PRSS3; 28, TOLLIP;
28, NFKB1; 28, CNN2; 33, GALT; 33, DTYMK; 33, RAD18; 33, CTBP2; 33, PGR; 33, BRD4; 33,
AK2; 33, ENTPD3; 33, DMAP1; 33, GTF2A1; 33, TRRAP; 33, DNAJC9; 33, NMRK2; 33, TAL1;
33, ENPP3; 33, ZGRF1; 33, TFF1; 33, CCDC157; 33, POLE3; 33, TAF7; 33, TTI1; 33, GUCY1A2;
33, YEATS4; 33, SMAD3; 33, CCDC101; 33, SAP130; 33, ARID4B; 33, KAT2B; 34, EGLN1; 34,
HSPA14; 34, ODC1; 34, MAP3K14; 34, AHNAK; 34, TNFRSF1A; 34, PSME3; 34, PSMB10; 34,
RCC1; 34, TNFRSF11A; 36, ALDH5A1; 36, SERPINE2; 36, KYNU; 36, ANG; 36, F13B; 36,
ALDH8A1; 36, SULT1C2; 37, SNX4; 37, CPLX1; 37, PPIF; 37, CPLX3; 37, SNAP47; 37,
ENSG00000168970; 37, RIMS1; 37, SCO2; 37, PPFIA4; 37, VDAC3; 37, DNAI1; 37, DOC2B; 37,
SYT4; 37, STX7; 37, CCDC114; 39, CAMKK1; 39, LEP; 39, PRKAB1; 39, PPM1A; 39, TSC2; 39,
MED13L; 39, PRKAA1; 40, ADH7; 40, ADH6; 40, ISCA2; 40, ACSS2; 40, PCCB; 40, ACADM; 40,
ADH1C; 40, GLUD1; 40, ACYP1; 40, DLST; 40, ALDH9A1; 40, LDHA; 40, AKR1B1; 40, GFPT2;
40, ACAT1; 42, SERP2; 42, TTC37; 42, PPP1R9A; 42, EIF4A2; 42, OSTC; 42, TUSC3; 42, CDNF;
42, RPL7; 42, RPN1; 42, BCAP29; 42, ATP6V0D1; 42, PSMC3; 42, SPCS2; 42, CCT5; 43,
UBAP2L; 43, RARA; 43, CBX7; 43, ASXL1; 43, POFUT1; 43, MBD6; 43, PHC2; 43, SUMO3; 48,
PTCRA; 48, HEYL; 48, HEY2; 48, SPEN; 48, MAML2; 48, AGO4; 49, FARSA; 49, LARS2; 49,
IARS2; 49, GTPBP3; 49, VARS; 49, PPT2; 49, EFHD2; 50, COL7A1; 50, CLGN; 50, HEBP2; 50,
SREBF2; 50, ANXA5; 50, PEF1; 50, SEC24D; 50, PDCD6;

TABLE 15G
EA all Immunochip genes (FIG. 10). Listed by: MCODE cluster, gene;

1, KRT25; 1, KRT17; 1, KRT16; 1, KRT15; 1, KRT14; 2, KRT9; 2, KRT12; 2, KRT33A; 2, KRT37;
2, KRT38; 2, KRT27; 2, KRT28; 2, KRT26; 2, KRT40; 2, KRT31; 2, KRT13; 2, KRT20; 2, KRT19;
2, KRT24; 2, KRT23; 3, ATG5; 3, IFIH1; 3, IRF4; 3, STAT1; 3, CASP8; 3, CASP10; 3, IRF8; 3,
CIAPIN1; 3, HERC5; 3, MX1; 3, OAS3; 3, IFI35; 3, IRF7; 3, IFI6; 3, OAS1; 3, OAS2; 3, IFI44L; 3,
IFIT1; 3, IRF5; 3, NLRX1; 3, IL21; 3, NFATC3; 3, IRGM; 3, SOCS7; 4, HSPBP1; 4, HSPE1; 4,
ATP5H; 4, ANAPC15; 4, CDC6; 4, UBE2C; 4, CDC20B; 4, PTTG1; 4, UBA52; 4, BRCA1; 4,
MCM6; 4, ILF3; 4, PARP2; 4, ITCH; 4, DUSP12; 4, TRIM25; 4, RPL19; 4, MRPS7; 4, RPS11; 4,
RPL27; 4, RPS20; 4, RPS14; 4, RPL24; 4, HSPA6; 4, ERCC1; 4, OIP5; 4, UBE2K; 4, KIF18A; 4,
RMI2; 4, TNKS; 4, RAD51; 4, SUMO2; 4, TOP2A; 4, NUSAP1; 4, C6orf106; 4, TNFAIP3; 4,
CCDC69; 4, TRAF3; 4, TK1; 4, CDK3; 4, GINS2; 4, PRIM2; 4, POLR2C; 4, CENPL; 4, CENPW; 4,
KNSTRN; 4, HIF3A; 4, EIF6; 4, SERBP1; 4, RPS25; 4, TUFM; 4, DOT1L; 4, RPL29; 4, EIF5A; 4,
QARS; 4, RAD23B; 4, FAM86C1; 4, MRPL38; 4, FBXW2; 4, SPRED2; 4, FIGNL1; 4, SLC9C2; 4,
KDM4B; 4, UBXN2B; 4, MKKS; 4, RPP14; 4, ZBP1; 4, REV3L; 4, KCNB1; 4, KCNG1; 4, NBR1; 4,
VRK1; 4, MRPL45; 4, OTUD3; 4, PDE12; 4, PDS5A; 4, USP14; 4, SKI; 4, TMEM258; 5, ACOX2;
5, NOS2; 5, ACTR2; 5, NCK2; 5, COTL1; 5, GRPEL1; 5, FCGR2A; 5, ARPC5; 5, ARPC2; 5,
LRRK2; 5, HCLS1; 5, ANP32B; 5, COBL; 5, ATP5L; 5, AIRE; 5, RAB1A; 5, APOE; 5, HDLBP; 5,
SAA1; 5, PCYOX1; 5, SDC1; 5, LCAT; 5, PLTP; 5, MAPT; 5, MAP3K11; 5, ARL1; 5, NSF; 5,
NRD1; 5, PPP2R1B; 5, HRAS; 5, PPP2CA; 5, ASIP; 5, VAMP2; 5, VDAC1; 5, RPTOR; 5, PPP5C; 5,
BCAR1; 5, PTPN1; 5, ITGAX; 5, LDHA; 5, BCKDK; 5, CAMK2D; 5, RORA; 5, MYC; 5, LAT; 5,
FCER1G; 5, CACNA1C; 5, RIMBP2; 5, RASGRP3; 5, CACNA2D3; 5, CACNA2D2; 5, CACNB1; 5,
IFNG; 5, FLNC; 5, FLNB; 5, STMN1; 5, CARM1; 5, TNFRSF21; 5, MED1; 5, RGL1; 5, FADS1; 5,
NCOA6; 5, NPAS2; 5, FDFT1; 5, CASKIN2; 5, CASP1; 5, IL1R2; 5, CCL22; 5, IL12A; 5, IL10; 5,
SMARCE1; 5, IL2; 5, CD80; 5, CD86; 5, IL2RA; 5, MAP4K4; 5, SEC24C; 5, DNM3; 5, IL1R1; 5,
CTLA4; 5, RNASE6; 5, STX4; 5, CELF2; 5, CPLX3; 5, STX1B; 5, LMAN1L; 5, SNAP47; 5, IPO8;
5, GDI2; 5, SCRIB; 5, NRG3; 5, EXOC2; 5, F5; 5, SEC16A; 5, TRAPPC4; 5, IFI30; 5, NFATC2; 5,
TNPO1; 5, SELL; 5, SH2D4A; 5, RAPGEF5; 5, IKZF3; 5, RAB23; 5, IFNL1; 5, TYK2; 5, NCOA5;
5, TNC; 5, PLA2G15; 5, WNT3; 5, STK25; 5, TNIK; 5, SYN3; 7, APEX1; 7, GZMA; 7, GIMAP4; 7,
CD2; 7, GIMAP8; 7, GIMAP7; 7, ICOS; 7, CD6; 7, CD5; 7, GIMAP6; 7, GIMAP5; 7, GIMAP1; 8,
ITPR2; 8, FLT1; 8, PLCB2; 8, PLCB1; 8, PLCB3; 8, CASR; 8, DGKH; 8, DGKQ; 8, INPP5E; 8,
SYNJ2; 8, TMEM55B; 8, IPMK; 8, TRPC3; 9, CD38; 12, IPO5; 12, EIF3C; 12, EIF3CL; 12, DHX29;
12, EIF1; 13, SKIV2L2; 13, UTP23; 13, PINX1; 13, HGH1; 13, TRMT11; 13, HEATR3; 14, ADNP;
14, RTF1; 14, CDC73; 14, BAG2; 14, SLU7; 14, TCF7; 14, E2F1; 14, TNPO3; 14, SYMPK; 14,
SUPT16H; 14, ELL; 14, RNF40; 14, PTRF; 14, CPSF1; 14, PAPOLG; 14, SRSF6; 14, NUP85; 14,
ZC3H4; 14, GTF3C3; 14, TSEN54; 14, THOC5; 14, CPSF3L; 14, RPAP2; 14, PUF60; 14, SNRPC;
14, GTF2H1; 14, XPO4; 14, GTF2H5; 14, TAF15; 14, SRRT; 14, PPIL3; 15, SMARCA4; 15,
JARID2; 15, DNMT3A; 15, GFI1; 15, DNMT3B; 15, DNMT3L; 16, ADORA3; 16, C1QB; 16, ANG;
16, RNASE1; 16, BLK; 16, EFNA1; 16, VAV2; 16, PTPRC; 16, IKZF1; 16, FCRLA; 16, NGEF; 16,
CD74; 16, RGS18; 16, TMEM176A; 16, GRAP2; 16, DYNLRB1; 16, IFT88; 16, NEDD4L; 16,
UBAP1; 16, IKZF4; 16, IKZF2; 17, AHSA2; 17, HSPD1; 17, PTGES3L-AARSD1; 17, PTGES3L;
17, TTC28; 17, PPIL6; 17, PPP1R14B; 18, COMMD7; 18, VAC14; 18, IL37; 18, JAK3; 18, CD40;
18, SOCS3; 18, ETS1; 18, IL6R; 18, IL12RB2; 18, PTPN2; 18, STAT4; 18, IL15RA; 18, IL27; 18,
PTPN7; 19, H3F3B; 19, H3F3A; 19, PELP1; 19, MORF4L1; 19, CCDC101; 19, KANSL1; 19,
TADA2B; 19, MSL1; 19, KAT8; 19, DR1; 19, SUDS3; 19, RNFT1; 19, EMC8; 19, FOXR1; 19,
NUPR1; 19, RPS6KA4; 20, IL7R; 20, IL12RB1; 20, IFNL2; 20, IL17D; 20, IL7; 20, SCARB2; 21,
ZC3HAV1; 21, TRIM24; 21, BCL2L11; 21, RARA; 21, CUX1; 21, STAU1; 22, BCAT1; 22, GSS;
22, GPX4; 22, CEBPB; 22, CHAC1; 22, GGT7; 22, PRDX6; 23, CLEC4D; 23, CLEC4E; 23,
CLEC6A; 25, PSMD5; 25, PSMB1; 25, PSMB10; 25, MTHFR; 26, ELMO2; 26, DOCK3; 26,
ELMO3; 27, CTRB1; 27, CTRB2; 27, CTRL; 28, PDGFRB; 28, BANK1; 28, ITGAM; 28, CCND1;
28, H2AFX; 28, CRYAB; 28, CCNO; 28, CCNJL; 28, SKP1; 28, FBXO31; 28, FBXL6; 28, FBXO40;
28, GP1BA; 28, ICAM4; 28, PLAU; 28, SKAP2; 28, MYCBP2; 29, DHX8; 29, DDX27; 29, RBM22;
29, SDE2; 29, HOXA5; 31, CAT; 31, ALDH2; 31, GLS; 31, GLUL; 31, LRTOMT; 31, AOC1; 31,
DLD; 33, ACSS2; 33, PCK1; 33, PDHB; 33, AFMID; 33, PPP6R1; 33, ENO3; 33, FAM120B; 33,
MED24; 34, ARRB2; 34, CSK; 34, CCR7; 34, SMO; 34, PEBP1; 34, CXCR5; 34, CXCR1; 34,
IL17RD; 34, OR6S1; 34, OR11H4; 34, CCR10; 34, GPR29; 34, CCR9; 34, ENG; 34, TCOF1; 34,
FCHO2; 34, FCHO1; 34, LYN; 34, BCL6; 34, ERBB2; 34, CD3E; 34, CTSA; 34, SGK223; 35,
ADAM15; 35, CDH1; 35, DLG1; 35, LAMC1; 35, LAMC2; 35, CDH17; 35, NUTF2; 35, PARD6A;
35, SNAI1; 35, ESRP2; 35, PTPRM; 35, NDRG1; 35, NCSTN; 35, DLL4; 35, NOTCH2; 35, DST;
35, MEGF9; 35, POGLUT1; 36, VPS18; 36, AP3D1; 36, ARL17B; 36, GAK; 36, UVRAG; 37,
PLEK; 37, WDFY4; 37, IL10RA; 37, CLECL1; 37, RANBP10; 37, RPAP1; 37, MANF; 38, ACBD3;
38, GOLGB1; 38, ANK3; 38, COPA; 38, CD44; 38, AP3B2; 38, ETV6; 38, SF3B1; 38, GGA3;

TABLE 15H
AsA all Immunochip genes (FIG. 10). Listed by: MCODE cluster, gene;

1, SLC1A2; 1, ZNF768; 1, UBQLN4; 1, PPIP5K2; 1, TCEA3; 1, UBL4A; 1, TFAM; 1, OXA1L; 1,
MAP4; 1, LMO2; 1, HIST1H4G; 1, TSPYL1; 1, HIST1H1T; 1, TMA7; 1, TAF7; 1, HCFC1; 1, PHF2;
1, HIST1H2AG; 1, HIST1H4I; 1, HIST1H4H; 1, HIST1H1E; 1, TNPO1; 1, MYSM1; 1, EIF1AD; 1,
RPS10; 1, RPS24; 1, RPSA; 1, EIF1; 1, KPNB1; 1, HSPA6; 1, RPP25; 1, SERBP1; 1, VBP1; 1,
SRP54; 1, RPL10; 1, ERCC8; 1, HIST1H4C; 1, CHAMP1; 1, NAA10; 1, TAF11; 1, CCNT2; 1,
H3F3A; 1, HIST1H3E; 1, HIST1H3H; 1, HIST1H3G; 1, RPS20; 1, MRPL4; 1, C12orf65; 1, RPL14;
1, RPL3; 1, RPL6; 1, POLR2B; 1, RPL10A; 1, POLR2C; 1, HIST1H3J; 1, SMARCE1; 1, PSMA6; 1,
EIF3E; 1, HIST1H2AC; 1, H2AFB1; 1, HIST1H2BO; 1, HIST1H2BJ; 1, HIST1H2BB; 1,
HIST1H2AD; 1, POU2F1; 1, SEC61A2; 1, PA2G4; 1, POLR2E; 2, REV3L; 2, PGGT1B; 2, FIGNL1;
2, POLB; 2, XRCC1; 2, RAD9B; 2, DNA2; 2, MPP1; 2, THADA; 2, UTP20; 2, NUP88; 2, XPO1; 2,
RTEL1; 2, ORC3; 2, MCM6; 2, RAD1; 2, DEPDC1B; 2, RMI2; 2, RFC2; 2, FEN1; 2, GINS2; 2,
NCAPG; 2, TOP2A; 2, RMI1; 2, FANCE; 2, C19orf40; 2, PTTG1; 3, USP34; 3, SKIL; 3, STRN4; 3,
PPP1R3B; 3, PPCDC; 3, SNRNP35; 3, PLCL2; 3, TROVE2; 3, RAI14; 3, KIAA1109; 3, SH2B3; 3,
MAP3K11; 3, MYO15A; 3, RASSF5; 3, PLAT; 3, IL21R; 3, GTF2I; 3, FLNC; 3, FBXW2; 3,
FBXW12; 3, FBXO9; 3, FBXL6; 3, FARSA; 3, SUFU; 3, HMGN1; 3, EIF4EBP2; 3, PTPN2; 3,
TYK2; 3, FBXO31; 3, TCERG1; 3, ZMAT5; 3, SNRPC; 3, STAT4; 3, RARS2; 3, DALRD3; 3,
DDX46; 3, SF3B2; 3, E2F2; 3, CDCA2; 3, CDC25A; 3, SLC22A5; 3, CCNJL; 3, IL1B; 3, IL1A; 3,
IL12A; 3, CCL22; 3, CX3CR1; 3, GPR29; 3, CXCR5; 3, SKP1; 3, CAST; 3, GRIN3B; 3, GRIK4; 3,
CACNA2D2; 3, RASGRP1; 3, RASGRF2; 3, CACNA1S; 3, RAB44; 3, CCL8; 3, C1QC; 3, C1QB; 3,
SLCO2B1; 3, C1QA; 3, SNRPA1; 3, TNIP2; 3, BRD7; 3, UVRAG; 3, DEF6; 3, IKZF1; 3, REL; 3,
BCKDK; 3, ITGAX; 3, ITGAD; 3, B7RP1; 3, UBE2E3; 3, TBCD; 3, WDR92; 3, COMMD8; 3,
ITGB1; 3, ITGAM; 3, RAB5C; 3, ANKRD17; 3, CDC16; 3, ANAPC16; 3, TRIM33; 3, CBX7; 3,
ANAPC1; 3, PPP2CA; 3, CDKN1A; 3, PPP2R3B; 3, IL2RA; 3, UBE2D1; 3, BCL10; 3, CTLA4; 3,
CD80; 3, MAP3K8; 3, PPP1CC; 3, AKAP11; 3, JAK2; 3, PITPNC1; 3, ATG7; 3, TRAK1; 3, VWF; 3,
CLCN6; 3, PDHX; 3, CSK; 3, AGTRAP; 3, CACNG3; 3, LRRK2; 4, KRT24; 4, KRT20; 4, KRT15;
4, KRT27; 4, KRT26; 4, KRT25; 4, KRT28; 4, KRT9; 4, KRT14; 4, CCDC8; 5, SIGIRR; 5, IL37; 5,
IL18R1; 5, IL11RA; 5, TAGAP; 5, IRF8; 5, GPR183; 5, GPR18; 5, DOCK5; 5, DOCK3; 5, NCF2; 5,
ICAM1; 5, MYO9B; 5, FCGR2A; 5, CLEC12A; 5, CD244; 5, CD247; 5, IL4; 5, LIF; 5, CCR7; 5,
RASGRP3; 5, WDFY4; 5, PLEK; 5, LY9; 5, PTPRC; 5, BCR; 5, RGS1; 5, ELMO1; 5, BLK; 5,
CD40; 5, MTCP1; 5, LAMTOR2; 5, NOS2; 5, PFKL; 5, ERBB3; 5, STAT3; 5, RAC2; 5, CDC37; 5,
ADCY7; 6, HIST1H1C; 6, HFE; 6, HDAC9; 6, KMT2B; 6, KLK3; 6, GPN3; 6, FLI1; 6, EGR2; 6,
HDAC7; 6, SETD8; 6, POLR3A; 6, KLK2; 6, REST; 6, CENPQ; 6, NAB1; 6, RNF40; 6, CDC73; 6,
NBN; 6, HIST1H2BK; 6, HIST1H2BN; 6, TCF7; 6, BCL9L; 6, MAPKAPK3; 6, SETD1A; 6,
BRCC3; 6, DKC1; 6, RARA; 6, AKT1; 6, KRI1; 6, POLR1D; 6, DDX55; 6, ABT1; 7, MCU; 7,
MRPL39; 7, EMC8; 7, TIMMDC1; 7, SDHC; 7, OMA1; 7, COX6B1; 7, UQCR10; 7, NDUFA9; 7,
CISD2; 7, NDUFAB1; 7, COX4I1; 7, COX6A2; 7, CISD1; 7, COX17; 7, HINT1; 7, COQ9; 8,
LCE4A; 8, LCE1C; 8, LCE3C; 8, LCE1D; 8, LCE1B; 8, KRTAP5-9; 9, TSPAN33; 9, C5; 9, C6; 10,
ADAMTSL1; 10, ADAMTSL3; 10, THSD7A; 10, ADAMTS3; 10, ADAMTS9; 10, ACAN; 11,
BEST1; 11, CLIC2; 11, ANO2; 11, GABRB1; 11, ANO1; 12, NIPSNAP1; 12, MGAT3; 12, IL1RN;
12, F11R; 12, PYDC1; 12, PYCARD; 12, ERC1; 12, TAB1; 12, PARD3; 12, PRKCI; 12, NOD2; 12,
IKBKB; 12, IRAK1; 12, ADCK3; 13, CDH23; 13, CDH8; 13, CDH17; 13, CDH12; 14, STX3; 14,
SIRPG; 14, POGLUT1; 14, ZMIZ1; 14, SSR2; 14, INPP5E; 14, MAML2; 14, MECP2; 14, LAT2; 14,
SYT1; 14, GAB3; 14, FURIN; 14, LILRA4; 14, TXK; 14, TEC; 14, EPHA8; 14, SPPL3; 14, USE1;
14, P4HA2; 14, PLOD1; 14, SEC16A; 14, MOBP; 14, CLEC1B; 14, RUNX3; 14, CD9; 14, COL9A1;
14, FCGR3B; 14, SH3BP2; 14, NUDT19; 14, TXNRD1; 14, PEX7; 14, CALU; 14, MANF; 14, F8;
14, STX4; 14, SSR4; 14, CNIH3; 14, PEX13; 14, BTN3A2; 14, SPP1; 14, SYK; 14, BANK1; 14,
HMGCLL1; 14, LAMB2; 14, SCARB1; 14, HSPG2; 14, CALR; 14, VIMP; 14, MSR1; 14, SDC4; 14,
GPC5; 14, KMO; 14, NCSTN; 14, DTX2; 14, EPHB2; 14, APH1B; 14, CD44; 14, COPA; 14,
TMED2; 14, ANK3; 14, AMT; 14, COL4A2; 14, IBSP; 14, ALPL; 14, ALDH9A1; 14, CNDP1; 14,
GLS; 14, ALDH2; 14, ITIH2; 14, CPS1; 14, APOB; 14, ALB; 14, LYN; 14, PIK3CG; 14, RPTOR;
14, NOTCH1; 14, PGM1; 14, ADSSL1; 14, GAD1; 14, ADO; 14, PDE4A; 14, STX1B; 14, ADAM23;
14, PEX3; 14, CAT; 14, ACSL6; 14, MMP13; 15, PLXNB1; 15, PLXNB3; 15, LIMK1; 15, PLXNA3;
15, NRP1; 15, DPYSL4; 16, IL36RN; 16, IL1RAPL1; 16, IL36B; 16, IL36A; 16, IL1F10; 18, GSN;
18, SLC25A6; 18, TIMM17A; 18, DNAJC15; 18, PARK2; 18, VDAC3; 18, VDAC1; 18, MFN2; 18,
ATG5; 19, RPS6KA2; 19, PRKD2; 19, PRKD1; 19, BATF; 19, CEBPG; 19, EIF2AK1; 19, ATF4; 19,
TRIB3; 20, NOA1; 20, WDR6; 20, TARS; 20, DPYD; 20, TDRD12; 20, CDKAL1; 20, ADA; 20,
BRIX1; 20, PES1; 20, LTV1; 20, WDR18; 21, XRCC4; 21, XRCC6BP1; 21, PMS1; 22, KCNU1; 22,
KCNMB2; 22, KCNMB4; 24, MGST3; 24, GSTA1; 24, GPX3; 25, NEB; 25, TNNI3; 25, MYBPC1;
25, PDLIM3; 25, DLG5; 25, POM121C; 25, WWOX; 25, THOC5; 25, KIF21B; 25, PCNXL3; 25,
SLU7; 25, CPSF1; 25, TPM1; 25, CNN2; 25, U2AF1L4; 25, CHERP; 25, CEP290; 25, DCLRE1B;
25, FGFR1OP; 25, CPSF6; 25, FUS; 25, PPP2R3C; 27, WDR73; 27, LIN9; 27, LIN37; 27, PSME3;
27, CDC6; 28, TRAF3IP1; 28, IFT81; 28, DYNC2LI1; 28, DYNC1I2; 28, DNAH10; 28, C1orf106;
28, GALNT2; 28, KIF5B; 28, ACTR1A; 29, MUT; 29, HLCS; 29, NIT1; 29, FHIT; 29, MCCC1; 29,
AUH; 30, NPR3; 30, PKD2; 30, CNGB1; 30, CNGA1; 30, RAB3IP; 30, CCDC146; 31, TLR5; 31,
TNIP1; 31, ETS1; 31, IKBKE; 31, CAMP; 31, TLR4; 31, TERT; 31, NFKB1; 32, TRPV4; 32,
PFKFB3; 32, PTRF; 32, PTPRK; 32, ZFYVE28; 32, IPCEF1; 32, LRRK1; 32, TNS4; 32, NTRK2; 32,
LPIN3; 32, HIP1; 32, KEAP1; 32, IGFBP3; 32, UBASH3A; 32, EFNA5; 32, BCAR1; 32,
ATP6V0A2; 32, ATP2A2; 32, UBE2D3; 32, ARHGEF7; 32, EPS15L1; 32, L1CAM; 32, ALDH1A3;
32, GRB10; 32, EGFR; 32, GNG2; 32, PRKCB;

Table 16. Cluster analysis of SLE SNP-predicted protein clusters using EA, AsA and shared genes from the Immunochip. Gene set enrichments for each cluster were determined using BIG-C (functional categories), I-SCOPE (cellular categories) and IPA (canonical pathways). Functional categories in bold-face indicate those the lowest P-value and highest odds ratio. P-values are from Fisher's exact test that measures the significance of overlap between analysis-ready genes in each cluster and genes within an annotation.

EA Immunochip pathways and functional/cellular enrichment

| | Functional | Cellular | | P |
|---|---|---|---|---|
| Cluter | categories | categories | IPA Canonical Pathway | value |
| 2 | IFN stimulated | | Interferon Signaling | 1.04E−06 |
| | genes | | Pathogen induced cytokine storm | 3.14E−06 |
| | PRRs | | signaling pathway | 3.62E−05 |
| | mRNA processing | | Role of RIG1-like receptors in | 2.02E−05 |
| | Endocytosis | | antiviral innate immunity | 5.44E−05 |
| | Ub & sumoylation | | Activation of IRF by cytosolic PRRs | |
| | | | Phagosome formation | |
| 7 | Immune signaling | Anergic or | TH1 pathway | 1.85E−13 |
| | Immune secreted | Activated T cell | Th1 and Th2 activation pathway | 3.02E−12 |
| | Immune cell surface | T, B & myeloid | TH2 pathway | 3.19E−09 |
| | | NK or T cell | T helper cell differentiation | 8.93E−09 |
| | | T & myeloid | CDX gastrointestinal cancer | 3.26E−08 |
| | | | signaling pathway | |
| 14 | Immune cell | Myeloid | Lipid antigen presentation by CD1 | 6.84E−03 |
| | surface | | Cytotoxic T lymphocyte-meduated | 8.90E−03 |
| | | | apoptosis of target cells | 9.21E−03 |
| | | | Coagulation system | 1.03E−02 |
| | | | Antiproliferative role of TOB in T cell | 1.13E−02 |
| | | | signaling | |
| | | | Oncostatin M signaling | |
| 18 | MHC class II | APCs | Leukotriene biosynthesis | 3.17E−03 |
| | | Monocytes | MIF-mediated glucocortocoid | 7.60E−03 |
| | | | regulation | 8.23E−03 |
| | | | Antigen presentation pathway | 9.29E−03 |
| | | | MIF regulation of Innate immunity | 1.47E−02 |
| | | | Eicosanoid signaling | |
| 11 | Pro-cell cycle | | Kinetochore metaphase signaling | 1.13E−04 |
| | Integrin signaling | | pathway | 1.38E−03 |
| | Golgi | | CTLA4 signaling in cytotoxic T | 2.41E−03 |
| | | | lymphocytes | 3.17E−03 |
| | | | CDK5 signaling | 6.16E−03 |
| | | | GP6 signaling pathway | |
| | | | Phagosome maturation | |
| 4 | mRNA splicing | | EIF2 signaling | 5.14E−07 |
| | | | Nucleotide excision repair pathway | 1.04E−03 |
| | | | NER pathway | 8.68E−03 |
| | | | Regulation of eIF4 and p70S6K | 2.15E−02 |
| | | | signaling | 2.29E−02 |
| | | | RAN signaling | |
| 17 | Proteasome | | Role of BRCA1 in DNA damage | 3.30E−05 |
| | General | | response | 5.46E−05 |
| | transcription | | Protein ubiquitination pathway | 1.53E−04 |
| | Pro-cell cycle | | Adipogenesis pathway | 1.74E−04 |
| | | | Hereditary breast cancer signaling | 3.47E−04 |
| | | | Nucleotide excision repair pathway | |
| 3 | Pro-cell cycle | | no enrichment | |
| 5 | Cytoplasm & | | CREB signaling in neurons | 8.58E−09 |
| | biochemistry | | P2Y purigenic receptor signaling | 6.04E−08 |
| | | | pathway | 6.56E−08 |
| | | | D-myo-inositol (1,4,5)-trisphosphate | 1.28E−07 |
| | | | biosynthesis | 3.88E−07 |
| | | | G protein signaling mediated by | |
| | | | Tubby | |
| | | | Superpathway of inositol phosphate | |
| | | | compounts | |
| 15 | Cytoplasm & | T & B cells | Guanosine nucleotides degradation | 1.12E−09 |
| | biochemistry | | III | 1.49E−09 |
| | | | Urate biosynthesis/inosine 5′ | 4.17E−09 |
| | | | phosphate degradation | 4.17E−09 |
| | | | Adenosine nucleotide degradation II | 1.26E−05 |
| | | | Purine nucleotides degradation II | |
| | | | NAD salvage pathway II | |
| 10 | General cell | | Circadian Rythym signaling | 1.30E−02 |
| | surface | | Breats cancer signaling by Stathmin 1 | 2.15E−02 |
| | | | CREB signaling in neurons | 2.19E−02 |
| | | | G alpha s | 4.16E−02 |
| 1 | General cell | | Glucocortocoid receptor signaling | 1.68E−18 |
| | surface | | Wound healing signaling pathway | 5.77E−03 |
| 8 | General | | Splicesomal cycle | 1.89E−02 |
| | transcription | | | |
| 22 | General | | Assembly of RNA polymerase II | 1.10E−02 |
| | transcription | | complex | |
| 19 | Endosome & | | Virus entry via endocytic pathways | 2.45E−04 |
| | vesicles | | Clathrin mediated endocytosis | 7.59E−04 |
| | Golgi | | signaling | |
| 12 | Nuclear receptor | | LXR/RXR activation | 1.14E−08 |
| | transcription | | FXR/RXR activation | 1.34E−08 |
| | | | Atherosclerosis signaling | 1.37E−06 |
| | | | Phospholipases | 1.20E−04 |
| | | | IL12 signaling and production in | 5.02E−04 |
| | | | macrophages | |
| 13 | Nuclear receptor | | PPARa/RXRa activation | 4.73E−05 |
| | transcription | | RAR activation | 4.95E−05 |
| | | | Estrogen receptor signaling | 2.35E−04 |
| | | | TR/RXR activation | 4.78E−04 |
| | | | Aryl hydrocarbon receptor signaling | 1.37E−03 |

AsA Immunochip pathways and functional/cellular enrichment

| | Functional | Cellular | | P |
|---|---|---|---|---|
| Cluster | categories | categories | IPA Canonical Pathway | value |
| 3 | PRRs | Myeloid | Role of PRRs in recognition of bacteria and | 4.09E−06 |
| | intracellular | | viruses | 8.74E−06 |
| | signaling | | NOD1/2 signaling pathway | 9.11E−06 |
| | | | Hepatic cholestasis | 1.80E−05 |
| | | | HER-2 signaling in breast cancer | 2.10E−05 |
| | | | IL12 signaling and production in | |
| | | | macrophages | |
| 6 | Immune | T & myeloid | IL33 signaling pathway | 3.25E−08 |
| | secreted | Myeloid | Pathogen induced cytokine storm signaling | 1.08E−06 |
| | | | pathway | 1.60E−06 |
| | | | HMGB1 signaling | 2.81E−06 |
| | | | Hepatic cholestasis | 3.19E−06 |
| | | | Role of cytokines in mediating | |
| | | | communication between immune cells | |
| 1 | Chromatin | | NAD signaling pathway | 1.88E−07 |
| | remodeling | | NER pathway | 2.00E−06 |
| | | | Transcriptional regulatory network in ES | 1.45E−05 |
| | | | cells | 1.30E−04 |
| | | | DNA methylation and transcriptional | 2.11E−04 |
| | | | repression signaling | |
| | | | Ferroptosis signaling pathway | |
| 2 | mRNA | | EIF2 signaling | 1.34E−10 |
| | processing | | Coronavirus pathogenesis pathway | 5.30E−07 |
| | General | | Adipogenesis pathway | 1.98E−05 |
| | transcription | | Prostate cancer signaling | 1.56E−04 |
| | Chromatin | | Assembly of RNA pol II complex | 2.29E−04 |
| | remodeling | | | |
| 10 | Autophagy | | Sirtuin signaling pathway | 2.23E−03 |
| | | | Role of JAK1, JAK2 and TYK2 in interferon | 6.59E−03 |
| | | | signaling | |
| 17 | mRNA splicing | | tRNA charging | 7.18E−04 |
| | Transpososn | | Spliceosomal cycle | 1.13E−03 |
| | control | | Uracil degradation II | 3.00E−03 |
| | Mito. DNA to | | Thymine degradation | 3.00E−03 |
| | RNA | | Adenine and adenosine salvage II | 7.00E−03 |
| | Nucleus & | | | |
| | nucleolus | | | |
| 4 | DNA repair | | BER pathway | 9.00E−08 |
| | | | Hereditary breast cancer signaling | 2.26E−07 |
| | | | Role of BRCA1 in DNA damage response | 1.00E−06 |
| | | | Role of CHK proteins in cell cycle | 2.41E−05 |
| | | | checkpoint control | 8.21E−05 |
| | | | DNA Double strand break repair by non- | |
| | | | homologous end joining | |
| 40 | Pro cell cyclce | | senescence pathway | 1.08E−10 |
| | Antiproliferation | | Estrogen-mediated S-phase entry signaling | 6.62E−08 |
| | | | Role of CHK proteins in cell cycle | 7.82E−07 |
| | | | checkpoint control | 1.21E−06 |
| | | | Mitotic roles of polo-like kinase | 1.27E−06 |
| | | | Cell cycle: G1/S checkpoint regulation | |
| 5 | DNA repair | | no enrichment | |
| 9 | OXPHOS | | Mitochondrial dysfunction | 2.30E−12 |
| | Mitochondria | | Oxidative phosphorylation | 1.58E−11 |
| | general | | HER-2 signaling in breast cancer | 2.30E−04 |
| | | | Sirtuin signaling pathway | 4.90E−04 |
| | | | Granzyme A signaling | 7.60E−04 |
| 19 | ROS protection | | Glutathione redox reactions I | 1.67E−09 |
| | | | Apelin adipocyte signaling pathway | 5.73E−08 |
| | | | Mitochondrial dysfunction | 3.00E−06 |
| | | | Glutathione mediated detoxification | 7.17E−06 |
| | | | Xenobiotic AHR signaling pathway | 4.00E−05 |
| 22 | Peroxisomes | | TNFR2 signaling | 1.00E−06 |
| | Ub & | | 4-1BB signaling in T lymphocytes | 1.20E−06 |
| | sumoylation | | TWAEK signaling | 1.60E−06 |
| | Intracellular | | IL17A signaling in fibroblasts | 1.73E−06 |
| | signaling | | April meduated signaling | 2.35E−06 |
| 30 | Glycolysis | | Histamine degradation | 4.13E−05 |
| | Cytoplasm & | | Fatty acid alpha oxidation | 4.13E−05 |
| | biochemistry | | Putrescine degradation III | 4.50E−05 |
| | | | Ethanol degradation IV | 5.89E−05 |
| | | | Tryptophan degradation | 6.89E−05 |
| 16 | Fatty acid | | Tryptophan degradation | 6.25E−05 |
| | biosynthesis | | LXR/RXR activation | 1.18E−04 |
| | Intracellular | | Atherosclerosis signaling | 1.29E−04 |
| | signaling | | FXR/RXR activation | 1.34E−04 |
| | | | NAD biosynthesis II | 2.73E−04 |
| 28 | Mitochondria | | Leucine degradation I | 1.90E−06 |
| | general | | Biotin carboxyl carrier protein assembly | 7.60E−04 |
| | | | Methylmalonyl pathway | 1.00E−03 |
| | | | 2-oxobutanoate degradation I | 1.27E−03 |
| | | | Isoleucine degradation I | 4.57E−03 |
| 41 | Mitochondria | | Urea cycle | 1.50E−03 |
| | general | | Superpathway of citrilline metabolism | 3.81E−03 |
| 11 | Secreted & | Myeloid | PPAR signaling | 1.70E−12 |
| | ECM | | p38 MAPK signaling | 3.12E−12 |
| | | | LXR/RXR activation | 3.54E−12 |
| | | | IL6 signaling | 4.51E−12 |
| | | | Granulocyte adhesion and diapedesis | 3.10E−11 |
| 23 | Integrin | Platelet | Axonal guidance system | 2.72E−08 |
| | Golgi | | Xenobiotic metabolism CAR signaling | 6.45E−09 |
| | General cell | | pathway | |
| | surface | | | |
| 12 | Secreted & | | Axonal guidance signaling | 6.57E−03 |
| | ECM | | Semaphorin neuronal repulsive signaling | 3.75E−02 |
| | Integrin | | pathway | |
| 25 | Immune | Monocytes | Pathogen induced cytokine storm signaling | 5.40E−06 |
| | secreted | | pathway | 3.34E−05 |
| | | | Granulocyte adhesion and diapedesis | 4.50E−05 |
| | | | Agranulocyte adhesion and diapedesis | 7.70E−05 |
| | | | Complement systems | 9.88E−04 |
| | | | STAT3 pathway | |
| 29 | Cytoskeleton | | Kinetochore metaphase signaling pathway | 3.70E−02 |
| 21 | RAS | | nNOS signaling in skeletal muscle cells | 6.00E−05 |
| | superfamily | | Netrin signaling | 1.37E−04 |
| | Transporters | | | |
| 13 | Intracellular | | Synaptic long term potentiation | 9.00E−07 |
| | signaling | | Dopamine feedback in cAMP signaling | 3.51E−06 |
| 15 | General cell | | Semaphorin signaling in neurons | 1.22E−11 |
| | surface | | Axonal guidance signaling | 5.37E−07 |
| 27 | Endoplasmic | | Insulin secretion signaling pathway | 7.82E−04 |
| | reticulum | | | |
| 7 | Transporters | | Prolactin signaling | 6.32E−08 |

Shared Immunochip pathways and functional/cellular enrichment

| | Functional | Cellular | | P |
|---|---|---|---|---|
| Cluster | categories | categories | IPA Canonical Pathway | value |
| 1 | PRRs | Myeloid | IL12 signaling and production in | 1.60E−05 |
| | Immue signaling | | macrophages | 3.00E−04 |
| | Immue cell | | NK cell signaling | 3.64E−03 |
| | surface | | T helper cell differentiation | 3.80E−03 |
| | | | TH2 pathway | 6.00E−03 |
| | | | TH1 and TH2 activation pathway | |
| 3 | Immue | Anergic or | Complement system | 1.22E−03 |
| | signaling | activated T cell | TCR signaling | 1.54E−03 |
| | Immue cell | T cell | Regulation of eIF4 and p70S6K signaling | 2.00E−03 |
| | surface | T, B & myeloid | Epoxysqualene biosynthesis | 2.80E−03 |
| | | T & myeloid | MSP-RON signaling pathway | 3.00E−03 |
| | | B cell | | |
| 14 | Immune | T & B cell | Role of | 9.27E−07 |
| | secreted | Myeloid cell | hypercytokinemia/hyperchemokinemia in | 9.93E−06 |
| | Immune | | the pathogenesis of flu | 1.21E−05 |
| | signaling | | Macrophage classical activation signaling | 1.21E−05 |
| | | | pathway | 1.75E−05 |
| | | | Macrophage alternative activation | |
| | | | signaling pathway | |
| | | | Role of JAK1, JAK2 and TYK2 in | |
| | | | interferon signaling | |
| | | | Interferon signaling | |
| 5 | Pro cell cycle | | Cell cycle: G2/M DNA damage checkpoint | 2.00E−04 |
| | DNA repair | | regulation | 2.46E−04 |
| | Ub & | | Cell cycle control of chromosomal | 2.80E−04 |
| | sumoylation | | replication | 2.84E−04 |
| | | | Mitotic roles of polo like kinase | 3.54E−04 |
| | | | Cell cycle: G1/S checkpoint regulation | |
| | | | Cyclins and cell cycle regulation | |
| 12 | General | | Cleavage & polyadenylation of pre-mRNA | 4.57E−03 |
| | transcription | | NER pathway | 1.33E−02 |
| | Nuclear receptor | | Role of OCT4 in mammalian ESC | 1.74E−02 |
| | transcription | | pluripotency | 1.89E−02 |
| | | | Assembly of RNA poI II complex | 1.95E−02 |
| | | | Glucocorticoid receptor signaling | |
| 9 | Mitochondria | Myeloid | Leukocyte extravasation signaling | 3.36E−04 |
| | general | | Apelin adipocyte signaling pathway | 1.97E−03 |
| | Intracellular | | Insulin receptor signaling | 4.48E−03 |
| | signaling | | Superoxide radicals degradation | 5.75E−03 |
| 2 | Secreted & | | Glucocorticoid receptor signaling | 2.19E−10 |
| | ECM | | | |
| 6 | WNT signaling | | no enrichment | |

TABLE 17
Cluster analysis of SLE SNP-predicted protein clusters using the entire cohort of EA
and AsA genes from the Immunochip. Gene set enrichments for each cluster were determinged
using BIG-C (functional categories), I-SCOPE (cellular catgories) and IPA (canonical
pathways). Functional categories in bold-face indicate those the lowest P-value and highest
odds ratio. P-values are from Fisher's exact test that measures the significance
of overlap between analysis-ready genes in each cluster and genes within an annotation.
EA Immunochip pathways and functional/cellular enrichment

| | Functional | | | P |
|---|---|---|---|---|
| Cluster | categories | Cellular categories | IPA Canonical Pathway | value |
| 4 | Pro cell | | EIF2 signaling | 4.90E−08 |
| | cycle | | | |
| | DNA repair | | Cell cycle control of chromosomal | 1.00E−06 |
| | | | replication | |
| | Mitochondrial DNA to RNA | | NER pathway | 2.17E−05 |
| | mRNA | | Protein ubiquitination pathway | 2.84E−05 |
| | processing | | | |
| | PRRs | | Coronavirus pathogenesis pathway | 5.12E−05 |
| 12 | mRNA | | Regulation of eIF4 and p70S6K | 5.70E−04 |
| | processing | | | |
| | | | EIF2 signaling | 9.00E−04 |
| | | | RAN signaling | 3.60E−03 |
| | | | Pyrimidine ribonucleotides | 8.00E−03 |
| | | | interconversion | |
| | | | Pyrimidine ribonucleotides de novo | 8.66E−03 |
| | | | biosynthesis | |
| 15 | Chromatin | | DNA methylation and transcriptional | 3.30E−04 |
| | remodeling | | repression signaling | |
| | | | HOTAIR regulatory pathway | 6.98E−04 |
| | | | Role of OCT4 in mammalian embryonic | 1.16E−02 |
| | | | stem cell pluripotency | |
| | | | Role of BRCA1 in DNA damage | 2.00E−02 |
| | | | response | |
| | | | Sumoylation pathway | 2.60E−02 |
| 19 | Chromatin | | DNA methylation and transcriptional | 2.25E−03 |
| | remodeling | | repression signaling | |
| | | | p38 MAPK Signaling | 2.58E−03 |
| | | | Macrophage alternative activation | 7.11E−03 |
| | | | signaling pathway | |
| | | | ERK/MAPK signaling | 8.00E−03 |
| | | | Role of IL17F in allergic inflammatory | 3.00E−02 |
| | | | airway diseases | |
| 28 | Pro cell | Erythrocyte | PTEN signaling | 1.60E−04 |
| | cycle | | | |
| | Immune | Platelet | Cell cycle: G1/S checkpoint regulation | 1.08E−03 |
| | signaling | T and myeloid cell | Pulmonary fibrosis ideopathic signaling | 1.54E−03 |
| | | | pathway | |
| | | | Cyclins and cell cycle regulation | 1.68E−03 |
| | | | Regulation of cellular mechanics by | 1.88E−03 |
| | | | calpain protease | |
| 25 | Proteasome | | FAT 10 signaling pathway | 5.05E−08 |
| | | | BAG2 signaling pathway | 1.73E−07 |
| | | | Inhibition of ARE-mediated mRNA | 1.26E−06 |
| | | | degradation pathway | |
| | | | Protein ubiquitination pathway | 6.07E−06 |
| | | | Huntington's disease pathway | 6.76E−06 |
| 13 | Antiproliferation | | Telomere extension by telomerase | 3.81E−03 |
| 29 | mRNA | | Spliceosomal cycle | 1.00E−02 |
| | splicing | | | |
| 38 | mRNA | | Coronavirus replication pathway | 1.70E−02 |
| | splicing | | | |
| | Golgi | | Spliceosomal cycle | 1.85E−02 |
| 5 | Immune | Myeloid cell | Synaptogenesis signaling pathway | 2.48E−10 |
| | secreted | | | |
| | Intracellular | | Cardiac hypertrophy signaling | 5.75E−10 |
| | signaling | | (enhanced) | |
| | | | SNARE signaling pathway | 8.30E−10 |
| | | | IL10 signaling | 2.79E−09 |
| | | | LXR/RXR activation | 6.29E−09 |
| 3 | Interferon stimulated genes | | Interferon signaling | 1.07E−12 |
| | PRRs | | Role of | 2.50E−10 |
| | | | hypercytokinemia/hyperchemokinemia | |
| | | | in the pathogenesis of flu | |
| | Pro- | | Role of PRRs in recognition of bacterial | 9.23E−09 |
| | apoptosis | | and viruses | |
| | | | Coronavirus pathogenesis pathway | 4.58E−08 |
| | | | Role of RIGI-like receptors in antiviral | 1.30E−07 |
| | | | innate immunity | |
| 16 | Immune | Treg | Ephrin A signaling | 9.60E−06 |
| | signaling | | | |
| | MHC class II | B cells | Fc Epislon RI signaling | 4.89E−03 |
| | Immune cell | APCs | GP6 Signaling pathway | 5.64E−03 |
| | surface | | | |
| | | | Ephrin receptor signaling | 1.30E−02 |
| | | | PI3K signaling in B lymphocytes | 1.40E−02 |
| 18 | Immune | B and myeloid cell | TH1 Pathway | 9.60E−06 |
| | signaling | | | |
| | Immune | T and myeloid cell | TH1 and TH2 activation pathway | 4.90E−03 |
| | secreted | | | |
| | Immune cell | | STAT3 pathway | 5.60E−03 |
| | surface | | | |
| | | | TH2 pathway | 1.38E−02 |
| | | | Pathogen induced cytokine storm | 1.48E−02 |
| | | | signaling pathway | |
| 37 | Immune cell | Myeloid cell | TH1 pathway | 2.76E−13 |
| | surface | | | |
| | | | STAT3 pathway | 3.17E−12 |
| | | | IL10 signaling | 1.10E−08 |
| 20 | Immune | NK/T cell | Role of JAK1 and JAK2 in cytokine | 1.18E−08 |
| | secreted | | signaling | |
| | Immune cell | Myeloid cell | IL7 signaling pathway | 1.66E−06 |
| | surface | | Cardiac hypertrophy signaling | 3.54E−02 |
| | | | (enhanced) | |
| | | | STAT3 pathway | 3.90E−02 |
| | | | PI3K/AKT signaling | 4.48E−02 |
| 23 | Immune cell | Monocyte | Phagosome formation | 8.30E−05 |
| | surface | | | |
| | | Myeloid cell | Role of PRRs in recognition of bacterial | 1.07E−04 |
| | | | and viruses | |
| 7 | Immune | T and B cell | Granzyme A signaling | 1.16E−04 |
| | signaling | | | |
| | Immune cell | Anergic or activated T | BER pathway | 3.21E−04 |
| | surface | cell | | |
| | | NK/T cell | tRNA splicing | 7.00E−04 |
| | | T, B and myeloid cell, | Primary immunodeficiency signaling | 2.55E−03 |
| | | T and myeloid cell | | |
| | | | SPINK1 general cancer pathway | 2.00E−02 |
| 22 | ROS | | gamma-glutamyl cycle | 4.56E−09 |
| | protection | | | |
| | | | Ferroptosis signaling pathway | 5.89E−06 |
| | | | Glutahione redox reactions I | 3.00E−05 |
| | | | Apelin adipocyte signaling pathway | 3.12E−04 |
| | | | Glutathione biosynthesis | 8.90E−04 |
| 34 | Immune | T, B and myeloid cell | Granulocyte adhesion and diapedesis | 4.15E−10 |
| | signaling | | | |
| | Immune cell | T and myeloid cell | Agranulocyte adhesion and diapedesis | 8.67E−10 |
| | surface | | | |
| | General cell | | FAK signaling | 2.85E−07 |
| | surface | | | |
| | | | Phagosome formation | 4.19E−05 |
| | | | GPCR signaling | 4.47E−05 |
| 35 | Integrin | | Regulation of EMT pathway | 5.00E−09 |
| | pathway | | | |
| | General cell | | Notch signaling | 3.10E−06 |
| | surface | | | |
| | | | Myelination signaling pathway | 3.66E−06 |
| | | | Regulation of EMT by growth factors | 1.19E−05 |
| | | | pathway | |
| | | | Regulation of EMT in development | 3.79E−05 |
| | | | pathway | |
| 31 | TCA cycle | | Histamine degradation | 1.58E−05 |
| | Mitochondria | | Ethanol degradation IV | 2.25E−05 |
| | I general | | Dopamine degradation | 3.72E−05 |
| | | | Noradrenaline and adrenaline | 5.00E−05 |
| | | | degradation | |
| | | | Glutamate signaling | 1.60E−04 |
| 33 | Fatty acid beta oxidation | | Acetate conversion to Acetyl-CoA | 1.70E−03 |
| | TCA cycle | | Tryptophan degradation to 2-amino- | 2.00E−03 |
| | | | 3carboxymuconate semialdehyde | |
| | General | | Acetyl-CoA biosynthesis I | 2.37E−03 |
| | transcription | | | |
| | | | Sirtuin signaling pathwau | 4.09E−03 |
| | | | NAD biosynthesis II | 4.40E−03 |
| 21 | Nuclear receptor trascription | | Coronavirus pathogenesis pathway | 1.09E−03 |
| | | | RAR activation | 1.10E−03 |
| | | | Glucocorticoid receptor signaling | 8.52E−03 |
| | | | Role of OCT4 in mammalian embryonic | 1.16E−02 |
| | | | stem cell pluripotency | |
| | | | MYC mediated apoptosis signaling | 1.26E−02 |
| 30 | Autophagy | | Role of MAPK in promoting the | 1.92E−02 |
| | | | pathogenesis of flu | |
| | Endoplasmic | | Iron homeostasis signaling pathway | 2.32E−02 |
| | reticulum | | | |
| 26 | RAS | | CXCR4 signaling | 1.50E−04 |
| | superfamily | | | |
| | Cytoskeleton | | Phagosome formation | 2.55E−03 |
| 36 | Endosome | | Clathrin-mediated endocytosis | 4.58E−04 |
| | & vesicle | | signaling | |
| | | | Autophagy | 4.94E−04 |
| 8 | Cytoplasm & biochemistry | | Superpathway of inositol phosphate | 1.40E−11 |
| | | | compounds | |
| | | | GPCR-mediated nutrient sensing in | 3.57E−09 |
| | | | enteroendocrine cells | |
| 1 & | Secreted & | | Glucocortocoid receptor signaling | 1.68E−18 |
| 2 | ECM | | | |

AsA Immunochip pathways and functional/cellular enrichment

| | Functional | Cellular | | |
|---|---|---|---|---|
| Cluster | categories | categories | IPA Canonical Pathway | P value |
| 1 | mRNA | | EIF2 signaling | 2.90E−11 |
| | processing | | | |
| | Chromatin | | NER pathway (enhanced) | 4.40E−10 |
| | remodeling | | | |
| | Mitochondrial DNA to RNA | | Assembly of RNA pol II complex | 2.60E−07 |
| | General | | Coronavirus pathogenesis pathway | 1.48E−06 |
| | transcription | | | |
| | | | NER pathway | 2.57E−06 |
| 2 | DNA repair | | Cell cycle control of chromosome replication | 2.00E−07 |
| | Pro cell cycle | | BER pathway | 9.10E−06 |
| | | | Role of BRCA1 in DNA damage response | 5.50E−05 |
| | | | NER pathway (enhanced) | 1.20E−04 |
| | | | Mismatch repair in eukaryotes | 1.26E−04 |
| 6 | Chromatin remodeling | | Adipogenesis pathway | 8.30E−07 |
| | Transcription factors | | Telomerase signaling | 1.00E−05 |
| | | | Prostate cancer signaling | 1.30E−05 |
| | | | Hereditary breast cancer signaling | 3.00E−05 |
| | | | NAD signaling pathway | 3.80E−05 |
| 20 | Transposon control | T & B cells | Uracil degradation II | 1.20E−03 |
| | | | Thymine degradation | 1.20E−03 |
| | | | Adenine and adenosine salvage III | 3.00E−03 |
| 21 | DNA repair | | DNA double strand break repair by non- | 3.55E−03 |
| | | | homologous end joining | |
| 27 | | | Cell cycle control of chromosome replication | 1.20E−02 |
| | | | FAT10 signaling pathway | 1.20E−02 |
| 25 | Proteasome | | Cleavage and polyadenylation of pre-mRNA | 5.00E−05 |
| | | | Spliceosomal cycle | 8.65E−04 |
| 3 | Immune | Anergic or | PI3K/AKT signaling | 5.50E−12 |
| | secreted | Activated T cell | | |
| | Integrin | T and B cell | Senescence pathway | 6.60E−11 |
| | immune | Myeloid cell | STAT3 pathway | 2.10E−12 |
| | signaling | | | |
| | Ubiquitylation | Monocyte | Cardiac hypertrophy signaling (enhanced) | 4.87E−10 |
| | & sumylation | | | |
| | mRNA | T and | Molecular mechanisms of cancer | 2.70E−10 |
| | splicing | myeloid cell | | |
| 12 | PRRs | Myeloid cell | Hepatic cholestasis | 1.31E−06 |
| | | | TLR signaling | 5.60E−06 |
| | | | Role of macrophages, fibrobalsts and | 1.17E−05 |
| | | | endothelial cells in RA | |
| | | | Hepatic fibrosis/hepatic stellate cell activation | 3.00E−05 |
| | | | Acute phase response signaling | 7.46E−05 |
| 5 | Immune secreted | NK/T cell | Pathogen induced cytokine storm signaling | 2.28E−12 |
| | | | pathway | |
| | Immune cell | Myeloid cell | Glucocorticoid receptor signaling | 1.26E−10 |
| | surface | | | |
| | | | STAT3 pathway | 5.50E−10 |
| | | | Differential regulation of cytokine production in | 2.08E−09 |
| | | | intestinal epithelial cells by IL17A and IL17F | |
| | | | Cardiac hypertrophy signaling (enhanced) | 2.62E−09 |
| 9 | Immune secreted | Monocyte | Complement systems | 1.00E−12 |
| 31 | PRRs | T and | NOD1/2 signaling pathway | 2.70E−07 |
| | | myeloid cell | | |
| | Intracellular | Neutrophil | iNOS signaling | 4.12E−07 |
| | signaling | | | |
| | Intracellular | T cell | Angiopoietin signaling | 1.78E−06 |
| | signaling | | | |
| | | | TREM1 signaling | 1.85E−06 |
| | | | TLR signaling | 1.90E−06 |
| 5 | Immune cell | Myeloid cell | HMGB1 signaling | 7.90E−08 |
| | surface | | | |
| | RAS superfamily | Anergic or | Macrophage classical activation signaling | 1.44E−06 |
| | | Activated T cell | pathway | |
| | | B & myeloid | Production of NO and ROS in macrophages | 2.78E−06 |
| | | B cell, T & | NK cell signaling | 3.30E−06 |
| | | myeloid | | |
| | | T. B & | CDX gastrointestinal cancer signaling pathway | 3.66E−06 |
| | | myeloid | | |
| 7 | OXPHOS | | Oxidative phosphorylation | 4.30E−15 |
| | Mitochondrial TCA cycle | | Mitochondrial dysfunction | 6.00E−13 |
| | Mitochondrial | | HER-2 signaling in breast cancer | 1.80E−05 |
| | general | | | |
| | | | Estrogen receptor signaling | 1.77E−04 |
| | | | Sirtuin signaling | 1.13E−03 |
| 24 | ROS | | Glutathione redox reactions I | 1.67E−09 |
| | protection | | | |
| | | | Apelin adipocyte signaling pathway | 5.70E−08 |
| | | | Mitochondrial dysfunction | 3.10E−06 |
| | | | Glutathione mediated detoxification | 7.17E−06 |
| | | | xenobiotic metablism AHR signaling pathway | 4.00E−05 |
| 14 | Peroxisome | NK/T cell | Notch signaling | 4.60E−09 |
| | Glycolysis | Monocyte | Role of osteoclasts in RA signaling pathway | 1.70E−06 |
| | Integrin | | IL15 production | 5.54E−06 |
| | ER | | GP6 signaling pathway | 6.66E−06 |
| | Immune | | Sperm motility | 4.00E−06 |
| | signaling | | | |
| 32 | Glycolysis | | Axonal guidance signaling | 7.00E−07 |
| | Integrin | | Thrombin signaling | 8.69E−05 |
| | General cell | | Sperm motility | 1.45E−04 |
| | surface | | | |
| | | | Huntington′s disease signaling | 2.00E−04 |
| | | | CLEAR signaling pathway | 2.16E−04 |
| 18 | Mitochondria | | Sirtuin signaling pathway | 1.57E−06 |
| | general | | | |
| | Autophagy | | Necroptosis signaling pathway | 1.55E−05 |
| | | | Mitochondrial dysfunction | 1.64E−04 |
| 29 | Mitochondria | | Leucine degradation I | 1.94E−06 |
| | general | | | |
| | | | Biotin-carboxyl carrier protein assembly | 7.62E−04 |
| 16 | Secreted & ECM | Myeloid | PPAR signaling | 1.74E−12 |
| | | | Granulocyte adhesion and diapedesis | 3.11E−11 |
| 10 | Secreted & ECM | | Axonal duidance signaling | 6.57E−03 |
| | Integrin | | Semaphorin neuronal repulsive signaling | 3.75E−02 |
| | | | pathway | |
| 4 | Secreted & ECM | | Glucocorticoid receptor signaling | 3.00E−11 |
| 19 | Intracellular | | Opiod signaling pathway | 5.58E−05 |
| | signaling | | | |
| | | | Role of IL17F in allergic inflammatory airway | 8.00E−05 |
| | | | disease | |
| | | | Role of macrophages, fibroblasts and | 9.26E−05 |
| | | | endothelial cells in RA | |
| 15 | General cell | | Semaphorin signaling in neurons | 5.80E−13 |
| | surface | | | |
| | | | Semaphorin neuronal replusive signaling | 5.79E−11 |
| | | | pathway | |
| | | | Axonal guidance signaling | 2.70E−08 |
| 22 | Transporters | | Prolactin signaling | 1.25E−06 |
| 11 | Transporters | | Glutathione redox reactions I | 6.10E−03 |
| | | | Apelin adipocyte signaling pathway | 1.90E−02 |
| 28 | Cytoskeleton | | Kinetochore metaphase signaling pathway | 3.70E−02 |

TABLE 18
AsA validation GWAS SNPs and their genomic and functional designation.
Listed by: SNP, Genomic annotation, Functional category;

rs10245913, 3′UTR, Coding; rs1045960, 3′UTR, Coding; rs1048783, 3′UTR, Coding; rs1049281,
3′UTR, Coding; rs10859101, 3′UTR, Coding; rs10903080, 3′UTR, Coding; rs11224565, 3′UTR,
Coding; rs1128334, 3′UTR, Coding; rs1136284, 3′UTR, Coding; rs11489, 3′UTR, Coding;
rs11539780, 3′UTR, Coding; rs11559013, 3′UTR, Coding; rs13447557, 3′UTR, Coding; rs16870907,
3′UTR, Coding; rs17254634, 3′UTR, Coding; rs2066951, 3′UTR, Coding; rs2071128, 3′UTR, Coding;
rs241451, 3′UTR, Coding; rs3077, 3′UTR, Coding; rs3117440, 3′UTR, Coding; rs3129302, 3′UTR,
Coding; rs3733370, 3′UTR, Coding; rs3795501, 3′UTR, Coding; rs3802735, 3′UTR, Coding; rs3830,
3′UTR, Coding; rs3853839, 3′UTR, Coding; rs426922, 3′UTR, Coding; rs449074, 3′UTR, Coding;
rs4552421, 3′UTR, Coding; rs4746971, 3′UTR, Coding; rs4849234, 3′UTR, Coding; rs4937333,
3′UTR, Coding; rs494003, 3′UTR, Coding; rs6673, 3′UTR, Coding; rs702348, 3′UTR, Coding;
rs758799, 3′UTR, Coding; rs7627498, 3′UTR, Coding; rs7756516, 3′UTR, Coding; rs8177834, 3′UTR,
Coding; rs8193023, 3′UTR, Coding; rs9276558, 3′UTR, Coding; rs9469341, 3′UTR, Coding;
rs948992, 3′UTR, Coding; rs9502116, 3′UTR, Coding; rs9585461, 3′UTR, Coding; rs9846199, 3′UTR,
Coding; rs11546678, 5′UTR, Coding; rs12287284, 5′UTR, Coding; rs17833769, 5′UTR, Coding;
rs1887428, 5′UTR, Coding; rs2295364, 5′UTR, Coding; rs2523506, 5′UTR, Coding; rs6903617,
5′UTR, Coding; rs806975, 5′UTR, Coding; rs922483, 5′UTR, Coding; rs9391734, 5′UTR, Coding;
rs1041981, Missense, Coding; rs1042190, Missense, Coding; rs10936599, Missense, Coding;
rs11235604, Missense, Coding; rs12419635, Missense, Coding; rs1249958, Missense, Coding;
rs2071596, Missense, Coding; rs2229094, Missense, Coding; rs2230926, Missense, Coding;
rs2301626, Missense, Coding; rs2303007, Missense, Coding; rs2305772, Missense, Coding;
rs28934585, Missense, Coding; rs4818950, Missense, Coding; rs5988, Missense, Coding; rs7097397,
Missense, Coding; rs7742542, Missense, Coding; rs1573649, Nonsense, Coding; rs494620, Nonsense,
Coding; rs6781844, Nonsense, Coding; rs1061357, Synonymous, Coding; rs11575839, Synonymous,
Coding; rs11623422, Synonymous, Coding; rs11767629, Synonymous, Coding; rs1378, Synonymous,
Coding; rs17184114, Synonymous, Coding; rs2071551, Synonymous, Coding; rs2073148,
Synonymous, Coding; rs2240335, Synonymous, Coding; rs241441, Synonymous, Coding; rs3134942,
Synonymous, Coding; rs3738476, Synonymous, Coding; rs453098, Synonymous, Coding; rs1548555,
Splice region, Non-coding; rs10034922, Intergenic, Non-coding; rs10066135, Intergenic, Non-coding;
rs10103079, Intergenic, Non-coding; rs10276619, Intergenic, Non-coding; rs10807113, Intergenic,
Non-coding; rs11697848, Intergenic, Non-coding; rs11748666, Intergenic, Non-coding; rs11757058,
Intergenic, Non-coding; rs12337277, Intergenic, Non-coding; rs12351527, Intergenic, Non-coding;
rs12526605, Intergenic, Non-coding; rs12640231, Intergenic, Non-coding; rs12798614, Intergenic,
Non-coding; rs13375357, Intergenic, Non-coding; rs154977, Intergenic, Non-coding; rs1568412,
Intergenic, Non-coding; rs17005911, Intergenic, Non-coding; rs17214519, Intergenic, Non-coding;
rs172274, Intergenic, Non-coding; rs2047391, Intergenic, Non-coding; rs2107189, Intergenic, Non-
coding; rs2202219, Intergenic, Non-coding; rs2213572, Intergenic, Non-coding; rs2585422,
Intergenic, Non-coding; rs2621322, Intergenic, Non-coding; rs2621323, Intergenic, Non-coding;
rs2621331, Intergenic, Non-coding; rs2647050, Intergenic, Non-coding; rs2722467, Intergenic, Non-
coding; rs2856705, Intergenic, Non-coding; rs2856718, Intergenic, Non-coding; rs2857114,
Intergenic, Non-coding; rs2858308, Intergenic, Non-coding; rs28724900, Intergenic, Non-coding;
rs3093671, Intergenic, Non-coding; rs3093998, Intergenic, Non-coding; rs3130542, Intergenic, Non-
coding; rs454746, Intergenic, Non-coding; rs5029394, Intergenic, Non-coding; rs529552, Intergenic,
Non-coding; rs6703281, Intergenic, Non-coding; rs6910071, Intergenic, Non-coding; rs7039011,
Intergenic, Non-coding; rs7076718, Intergenic, Non-coding; rs7518551, Intergenic, Non-coding;
rs7744293, Intergenic, Non-coding; rs7748472, Intergenic, Non-coding; rs7763262, Intergenic, Non-
coding; rs7769979, Intergenic, Non-coding; rs7770024, Intergenic, Non-coding; rs7774452,
Intergenic, Non-coding; rs8188001, Intergenic, Non-coding; rs9258559, Intergenic, Non-coding;
rs9264508, Intergenic, Non-coding; rs9267431, Intergenic, Non-coding; rs9275388, Intergenic, Non-
coding; rs9275390, Intergenic, Non-coding; rs9275393, Intergenic, Non-coding; rs9275406,
Intergenic, Non-coding; rs9275407, Intergenic, Non-coding; rs9275418, Intergenic, Non-coding;
rs9275424, Intergenic, Non-coding; rs9275428, Intergenic, Non-coding; rs9275439, Intergenic, Non-
coding; rs9275464, Intergenic, Non-coding; rs9275793, Intergenic, Non-coding; rs9276017,
Intergenic, Non-coding; rs9276162, Intergenic, Non-coding; rs9276461, Intergenic, Non-coding;
rs9277334, Intergenic, Non-coding; rs9502981, Intergenic, Non-coding; rs9842140, Intergenic, Non-
coding; rs9975425, Intergenic, Non-coding; rs1001455, Intergenic, Non-coding; rs10043883,
Intergenic, Non-coding; rs10058830, Intergenic, Non-coding; rs10086746, Intergenic, Non-coding;
rs10121985, Intergenic, Non-coding; rs10172745, Intergenic, Non-coding; rs10489265, Intergenic,
Non-coding; rs10517986, Intergenic, Non-coding; rs1068156, Intergenic, Non-coding; rs10798269,
Intergenic, Non-coding; rs10826460, Intergenic, Non-coding; rs10882672, Intergenic, Non-coding;
rs10888312, Intergenic, Non-coding; rs10910196, Intergenic, Non-coding; rs10952049, Intergenic,
Non-coding; rs10952629, Intergenic, Non-coding; rs10996405, Intergenic, Non-coding; rs11005650,
Intergenic, Non-coding; rs11088840, Intergenic, Non-coding; rs11162536, Intergenic, Non-coding;
rs11196522, Intergenic, Non-coding; rs11204176, Intergenic, Non-coding; rs11213783, Intergenic,
Non-coding; rs11235667, Intergenic, Non-coding; rs1145423, Intergenic, Non-coding; rs11710313,
Intergenic, Non-coding; rs11744430, Intergenic, Non-coding; rs11895901, Intergenic, Non-coding;
rs11910489, Intergenic, Non-coding; rs11918863, Intergenic, Non-coding; rs11926801, Intergenic,
Non-coding; rs11954949, Intergenic, Non-coding; rs1202519, Intergenic, Non-coding; rs12152285,
Intergenic, Non-coding; rs12161091, Intergenic, Non-coding; rs12200632, Intergenic, Non-coding;
rs12202739, Intergenic, Non-coding; rs12206125, Intergenic, Non-coding; rs12247850, Intergenic,
Non-coding; rs12280203, Intergenic, Non-coding; rs1234317, Intergenic, Non-coding; rs12350632,
Intergenic, Non-coding; rs12366033, Intergenic, Non-coding; rs12397712, Intergenic, Non-coding;
rs1246397, Intergenic, Non-coding; rs12467926, Intergenic, Non-coding; rs12480904, Intergenic,
Non-coding; rs12495067, Intergenic, Non-coding; rs12507862, Intergenic, Non-coding; rs12519302,
Intergenic, Non-coding; rs12542661, Intergenic, Non-coding; rs12633549, Intergenic, Non-coding;
rs13020448, Intergenic, Non-coding; rs13064646, Intergenic, Non-coding; rs13092494, Intergenic,
Non-coding; rs13099895, Intergenic, Non-coding; rs13118223, Intergenic, Non-coding; rs13148043,
Intergenic, Non-coding; rs13187024, Intergenic, Non-coding; rs1319208, Intergenic, Non-coding;
rs13195441, Intergenic, Non-coding; rs13197350, Intergenic, Non-coding; rs13211318, Intergenic,
Non-coding; rs13229173, Intergenic, Non-coding; rs13233677, Intergenic, Non-coding; rs13277113,
Intergenic, Non-coding; rs1342032, Intergenic, Non-coding; rs13433791, Intergenic, Non-coding;
rs1360555, Intergenic, Non-coding; rs1371438, Intergenic, Non-coding; rs1372389, Intergenic, Non-
coding; rs1382271, Intergenic, Non-coding; rs1391537, Intergenic, Non-coding; rs1513172,
Intergenic, Non-coding; rs156195, Intergenic, Non-coding; rs1562895, Intergenic, Non-coding;
rs1578624, Intergenic, Non-coding; rs1600249, Intergenic, Non-coding; rs1626392, Intergenic, Non-
coding; rs16829738, Intergenic, Non-coding; rs16870488, Intergenic, Non-coding; rs16876855,
Intergenic, Non-coding; rs17028314, Intergenic, Non-coding; rs17069252, Intergenic, Non-coding;
rs17132781, Intergenic, Non-coding; rs17291128, Intergenic, Non-coding; rs17387185, Intergenic,
Non-coding; rs1922120, Intergenic, Non-coding; rs1935105, Intergenic, Non-coding; rs1948680,
Intergenic, Non-coding; rs1993321, Intergenic, Non-coding; rs2048830, Intergenic, Non-coding;
rs2373488, Intergenic, Non-coding; rs2409781, Intergenic, Non-coding; rs2436919, Intergenic, Non-
coding; rs2546863, Intergenic, Non-coding; rs2590621, Intergenic, Non-coding; rs2618455,
Intergenic, Non-coding; rs2618476, Intergenic, Non-coding; rs2618479, Intergenic, Non-coding;
rs26192, Intergenic, Non-coding; rs2621416, Intergenic, Non-coding; rs2736340, Intergenic, Non-
coding; rs276368, Intergenic, Non-coding; rs282742, Intergenic, Non-coding; rs2831254, Intergenic,
Non-coding; rs2856664, Intergenic, Non-coding; rs2857204, Intergenic, Non-coding; rs2857210,
Intergenic, Non-coding; rs2857211, Intergenic, Non-coding; rs2857595, Intergenic, Non-coding;
rs2857700, Intergenic, Non-coding; rs2858312, Intergenic, Non-coding; rs2867656, Intergenic, Non-
coding; rs3105839, Intergenic, Non-coding; rs3113763, Intergenic, Non-coding; rs3132681,
Intergenic, Non-coding; rs3135005, Intergenic, Non-coding; rs326814, Intergenic, Non-coding;
rs34889541, Intergenic, Non-coding; rs35866326, Intergenic, Non-coding; rs422089, Intergenic, Non-
coding; rs4284147, Intergenic, Non-coding; rs441576, Intergenic, Non-coding; rs4711176, Intergenic,
Non-coding; rs4713172, Intergenic, Non-coding; rs4723604, Intergenic, Non-coding; rs4725496,
Intergenic, Non-coding; rs487726, Intergenic, Non-coding; rs4917129, Intergenic, Non-coding;
rs4917131, Intergenic, Non-coding; rs4928191, Intergenic, Non-coding; rs4971212, Intergenic, Non-
coding; rs547977, Intergenic, Non-coding; rs602457, Intergenic, Non-coding; rs6096689, Intergenic,
Non-coding; rs6128926, Intergenic, Non-coding; rs6425217, Intergenic, Non-coding; rs6456832,
Intergenic, Non-coding; rs6457580, Intergenic, Non-coding; rs6457658, Intergenic, Non-coding;
rs6457661, Intergenic, Non-coding; rs6559061, Intergenic, Non-coding; rs6591078, Intergenic, Non-
coding; rs660895, Intergenic, Non-coding; rs6671832, Intergenic, Non-coding; rs6700383, Intergenic,
Non-coding; rs6802343, Intergenic, Non-coding; rs6812030, Intergenic, Non-coding; rs6860468,
Intergenic, Non-coding; rs6866752, Intergenic, Non-coding; rs6877314, Intergenic, Non-coding;
rs6882535, Intergenic, Non-coding; rs6901084, Intergenic, Non-coding; rs6925972, Intergenic, Non-
coding; rs6927849, Intergenic, Non-coding; rs6932056, Intergenic, Non-coding; rs6937545,
Intergenic, Non-coding; rs6987057, Intergenic, Non-coding; rs6993775, Intergenic, Non-coding;
rs7009832, Intergenic, Non-coding; rs7031422, Intergenic, Non-coding; rs704840, Intergenic, Non-
coding; rs7078783, Intergenic, Non-coding; rs7382159, Intergenic, Non-coding; rs7526891,
Intergenic, Non-coding; rs7614652, Intergenic, Non-coding; rs7618000, Intergenic, Non-coding;
rs7701515, Intergenic, Non-coding; rs7704938, Intergenic, Non-coding; rs7705218, Intergenic, Non-
coding; rs7726414, Intergenic, Non-coding; rs7729194, Intergenic, Non-coding; rs7754969,
Intergenic, Non-coding; rs7788569, Intergenic, Non-coding; rs7788572, Intergenic, Non-coding;
rs8129911, Intergenic, Non-coding; rs822745, Intergenic, Non-coding; rs844644, Intergenic, Non-
coding; rs850456, Intergenic, Non-coding; rs900979, Intergenic, Non-coding; rs903943, Intergenic,
Non-coding; rs911178, Intergenic, Non-coding; rs9258788, Intergenic, Non-coding; rs9262560,
Intergenic, Non-coding; rs9270986, Intergenic, Non-coding; rs9271055, Intergenic, Non-coding;
rs9271366, Intergenic, Non-coding; rs9271775, Intergenic, Non-coding; rs9275245, Intergenic, Non-
coding; rs9275312, Intergenic, Non-coding; rs9275328, Intergenic, Non-coding; rs9275330,
Intergenic, Non-coding; rs9275333, Intergenic, Non-coding; rs9275915, Intergenic, Non-coding;
rs9368502, Intergenic, Non-coding; rs9445221, Intergenic, Non-coding; rs9473492, Intergenic, Non-
coding; rs9495845, Intergenic, Non-coding; rs9496060, Intergenic, Non-coding; rs9501426,
Intergenic, Non-coding; rs953028, Intergenic, Non-coding; rs9609463, Intergenic, Non-coding;
rs978929, Intergenic, Non-coding; rs9845508, Intergenic, Non-coding; rs9846187, Intergenic, Non-
coding; rs9852178, Intergenic, Non-coding; rs9852405, Intergenic, Non-coding; rs9863292,
Intergenic, Non-coding; rs986399, Intergenic, Non-coding; rs9872785, Intergenic, Non-coding;
rs9883825, Intergenic, Non-coding; rs9999874, Intergenic, Non-coding; rs10001879, Intron, Non-
coding; rs10007253, Intron, Non-coding; rs10031986, Intron, Non-coding; rs10034409, Intron, Non-
coding; rs10040019, Intron, Non-coding; rs10042987, Intron, Non-coding; rs10052333, Intron, Non-
coding; rs10055809, Intron, Non-coding; rs10057591, Intron, Non-coding; rs10085491, Intron, Non-
coding; rs10100115, Intron, Non-coding; rs10113553, Intron, Non-coding; rs10121357, Intron, Non-
coding; rs10123125, Intron, Non-coding; rs10160596, Intron, Non-coding; rs10168266, Intron, Non-
coding; rs10182929, Intron, Non-coding; rs10197709, Intron, Non-coding; rs10207131, Intron, Non-
coding; rs10214910, Intron, Non-coding; rs10216107, Intron, Non-coding; rs10218559, Intron, Non-
coding; rs10218623, Intron, Non-coding; rs10226732, Intron, Non-coding; rs1023449, Intron, Non-
coding; rs10250103, Intron, Non-coding; rs102518, Intron, Non-coding; rs10256306, Intron, Non-
coding; rs10267842, Intron, Non-coding; rs1028888, Intron, Non-coding; rs1038643, Intron, Non-
coding; rs10399874, Intron, Non-coding; rs10441664, Intron, Non-coding; rs10497041, Intron, Non-
coding; rs10497582, Intron, Non-coding; rs10499153, Intron, Non-coding; rs10516138, Intron, Non-
coding; rs10517254, Intron, Non-coding; rs10736424, Intron, Non-coding; rs10753267, Intron, Non-
coding; rs10791824, Intron, Non-coding; rs10807150, Intron, Non-coding; rs10830916, Intron, Non-
coding; rs10830996, Intron, Non-coding; rs10857650, Intron, Non-coding; rs10874449, Intron, Non-
coding; rs10901656, Intron, Non-coding; rs10935422, Intron, Non-coding; rs10979661, Intron, Non-
coding; rs11000258, Intron, Non-coding; rs11004662, Intron, Non-coding; rs11022074, Intron, Non-
coding; rs11026069, Intron, Non-coding; rs11031413, Intron, Non-coding; rs11032963, Intron, Non-
coding; rs1109771, Intron, Non-coding; rs11100014, Intron, Non-coding; rs11101535, Intron, Non-
coding; rs11101537, Intron, Non-coding; rs11118082, Intron, Non-coding; rs11128974, Intron, Non-
coding; rs11135570, Intron, Non-coding; rs11136611, Intron, Non-coding; rs11192156, Intron, Non-
coding; rs11249152, Intron, Non-coding; rs11254813, Intron, Non-coding; rs11263845, Intron, Non-
coding; rs11498370, Intron, Non-coding; rs11505605, Intron, Non-coding; rs11567714, Intron, Non-
coding; rs11573133, Intron, Non-coding; rs1157938, Intron, Non-coding; rs11679952, Intron, Non-
coding; rs11680343, Intron, Non-coding; rs11686127, Intron, Non-coding; rs117026326, Intron, Non-
coding; rs11709815, Intron, Non-coding; rs11712248, Intron, Non-coding; rs11719628, Intron, Non-
coding; rs11733406, Intron, Non-coding; rs11734340, Intron, Non-coding; rs11741929, Intron, Non-
coding; rs11746609, Intron, Non-coding; rs1175122, Intron, Non-coding; rs11754824, Intron, Non-
coding; rs11776830, Intron, Non-coding; rs11784845, Intron, Non-coding; rs11800609, Intron, Non-
coding; rs11804204, Intron, Non-coding; rs11889341, Intron, Non-coding; rs11917894, Intron, Non-
coding; rs11920533, Intron, Non-coding; rs1 1925129, Intron, Non-coding; rs11926726, Intron, Non-
coding; rs11932133, Intron, Non-coding; rs11933079, Intron, Non-coding; rs11933431, Intron, Non-
coding; rs1193787, Intron, Non-coding; rs11944579, Intron, Non-coding; rs1 1946839, Intron, Non-
coding; rs11948936, Intron, Non-coding; rs11949459, Intron, Non-coding; rs11953798, Intron, Non-
coding; rs11955321, Intron, Non-coding; rs1 1959133, Intron, Non-coding; rs11970564, Intron, Non-
coding; rs11970868, Intron, Non-coding; rs11971232, Intron, Non-coding; rs11979016, Intron, Non-
coding; rs11981568, Intron, Non-coding; rs11989012, Intron, Non-coding; rs11997593, Intron, Non-
coding; rs12003358, Intron, Non-coding; rs1202542, Intron, Non-coding; rs12026092, Intron, Non-
coding; rs12054287, Intron, Non-coding; rs12075005, Intron, Non-coding; rs12078339, Intron, Non-
coding; rs12082952, Intron, Non-coding; rs12083651, Intron, Non-coding; rs12095514, Intron, Non-
coding; rs12109018, Intron, Non-coding; rs12112578, Intron, Non-coding; rs12130544, Intron, Non-
coding; rs12132539, Intron, Non-coding; rs12153855, Intron, Non-coding; rs12154894, Intron, Non-
coding; rs12198173, Intron, Non-coding; rs12256635, Intron, Non-coding; rs12276773, Intron, Non-
coding; rs12293461, Intron, Non-coding; rs1233284, Intron, Non-coding; rs1234313, Intron, Non-
coding; rs1234315, Intron, Non-coding; rs12345414, Intron, Non-coding; rs12359596, Intron, Non-
coding; rs12365149, Intron, Non-coding; rs12401806, Intron, Non-coding; rs12403964, Intron, Non-
coding; rs12404160, Intron, Non-coding; rs12466158, Intron, Non-coding; rs12476060, Intron, Non-
coding; rs12499194, Intron, Non-coding; rs12501107, Intron, Non-coding; rs12515747, Intron, Non-
coding; rs12518265, Intron, Non-coding; rs12521169, Intron, Non-coding; rs12529247, Intron, Non-
coding; rs12633960, Intron, Non-coding; rs12644506, Intron, Non-coding; rs12649891, Intron, Non-
coding; rs12659024, Intron, Non-coding; rs12663608, Intron, Non-coding; rs12676149, Intron, Non-
coding; rs12676308, Intron, Non-coding; rs12719344, Intron, Non-coding; rs12726307, Intron, Non-
coding; rs12761196, Intron, Non-coding; rs12794220, Intron, Non-coding; rs1280946, Intron, Non-
coding; rs1287275, Intron, Non-coding; rs13001867, Intron, Non-coding; rs13010749, Intron, Non-
coding; rs13035366, Intron, Non-coding; rs13050595, Intron, Non-coding; rs13097956, Intron, Non-
coding; rs13105711, Intron, Non-coding; rs13126272, Intron, Non-coding; rs13130159, Intron, Non-
coding; rs13130621, Intron, Non-coding; rs13168551, Intron, Non-coding; rs13170924, Intron, Non-
coding; rs13172853, Intron, Non-coding; rs13174179, Intron, Non-coding; rs1317487, Intron, Non-
coding; rs13179010, Intron, Non-coding; rs1319898, Intron, Non-coding; rs13199524, Intron, Non-
coding; rs1320476, Intron, Non-coding; rs13206957, Intron, Non-coding; rs13215574, Intron, Non-
coding; rs132673, Intron, Non-coding; rs13275001, Intron, Non-coding; rs13314127, Intron, Non-
coding; rs13318819, Intron, Non-coding; rs13321848, Intron, Non-coding; rs13358038, Intron, Non-
coding; rs13358286, Intron, Non-coding; rs13361596, Intron, Non-coding; rs13375083, Intron, Non-
coding; rs13377162, Intron, Non-coding; rs13391903, Intron, Non-coding; rs13399735, Intron, Non-
coding; rs13407419, Intron, Non-coding; rs13414946, Intron, Non-coding; rs1341599, Intron, Non-
coding; rs13434010, Intron, Non-coding; rs1344645, Intron, Non-coding; rs1394796, Intron, Non-
coding; rs1426096, Intron, Non-coding; rs1431399, Intron, Non-coding; rs1431400, Intron, Non-
coding; rs1431401, Intron, Non-coding; rs1456893, Intron, Non-coding; rs1461131, Intron, Non-
coding; rs1474788, Intron, Non-coding; rs1476818, Intron, Non-coding; rs1485383, Intron, Non-
coding; rs1492909, Intron, Non-coding; rs1501913, Intron, Non-coding; rs1502747, Intron, Non-
coding; rs1513284, Intron, Non-coding; rs1530565, Intron, Non-coding; rs1547741, Intron, Non-
coding; rs1549020, Intron, Non-coding; rs1568854, Intron, Non-coding; rs1581774, Intron, Non-
coding; rs1583434, Intron, Non-coding; rs1604380, Intron, Non-coding; rs1609407, Intron, Non-
coding; rs16831742, Intron, Non-coding; rs16833215, Intron, Non-coding; rs16835865, Intron, Non-
coding; rs16840397, Intron, Non-coding; rs16871071, Intron, Non-coding; rs16876102, Intron, Non-
coding; rs16883819, Intron, Non-coding; rs16885926, Intron, Non-coding; rs16893651, Intron, Non-
coding; rs16894919, Intron, Non-coding; rs1689798, Intron, Non-coding; rs16911182, Intron, Non-
coding; rs16929202, Intron, Non-coding; rs16935778, Intron, Non-coding; rs16938746, Intron, Non-
coding; rs16988435, Intron, Non-coding; rs16990685, Intron, Non-coding; rs16993266, Intron, Non-
coding; rs16996395, Intron, Non-coding; rs17006749, Intron, Non-coding; rs17016165, Intron, Non-
coding; rs17025852, Intron, Non-coding; rs17046616, Intron, Non-coding; rs17057310, Intron, Non-
coding; rs1706143, Intron, Non-coding; rs17121972, Intron, Non-coding; rs1 7123728, Intron, Non-
coding; rs171329, Intron, Non-coding; rs17172433, Intron, Non-coding; rs1 7366139, Intron, Non-
coding; rs17394821, Intron, Non-coding; rs17455052, Intron, Non-coding; rs17475331, Intron, Non-
coding; rs17476188, Intron, Non-coding; rs174852, Intron, Non-coding; rs17587143, Intron, Non-
coding; rs17593228, Intron, Non-coding; rs17603856, Intron, Non-coding; rs17760881, Intron, Non-
coding; rs17794300, Intron, Non-coding; rs17801031, Intron, Non-coding; rs17846948, Intron, Non-
coding; rs1835826, Intron, Non-coding; rs1859838, Intron, Non-coding; rs1864331, Intron, Non-
coding; rs1866179, Intron, Non-coding; rs1876832, Intron, Non-coding; rs1884397, Intron, Non-
coding; rs1894407, Intron, Non-coding; rs1894408, Intron, Non-coding; rs1903501, Intron, Non-
coding; rs1925579, Intron, Non-coding; rs1950091, Intron, Non-coding; rs1980789, Intron, Non-
coding; rs2006165, Intron, Non-coding; rs2009453, Intron, Non-coding; rs2016705, Intron, Non-
coding; rs2042126, Intron, Non-coding; rs204267, Intron, Non-coding; rs204990, Intron, Non-coding;
rs204991, Intron, Non-coding; rs2051549, Intron, Non-coding; rs2063543, Intron, Non-coding;
rs2071349, Intron, Non-coding; rs2071351, Intron, Non-coding; rs2071353, Intron, Non-coding;
rs2071473, Intron, Non-coding; rs2071479, Intron, Non-coding; rs2071592, Intron, Non-coding;
rs2071652, Intron, Non-coding; rs2077180, Intron, Non-coding; rs2077580, Intron, Non-coding;
rs2100224, Intron, Non-coding; rs2139950, Intron, Non-coding; rs2156237, Intron, Non-coding;
rs2157338, Intron, Non-coding; rs2187823, Intron, Non-coding; rs2188582, Intron, Non-coding;
rs2207316, Intron, Non-coding; rs2207401, Intron, Non-coding; rs2215980, Intron, Non-coding;
rs2217128, Intron, Non-coding; rs2250243, Intron, Non-coding; rs2251824, Intron, Non-coding;
rs2256974, Intron, Non-coding; rs2267828, Intron, Non-coding; rs2273919, Intron, Non-coding;
rs2291875, Intron, Non-coding; rs2296330, Intron, Non-coding; rs2297550, Intron, Non-coding;
rs2301220, Intron, Non-coding; rs2301224, Intron, Non-coding; rs2301226, Intron, Non-coding;
rs2301271, Intron, Non-coding; rs2304010, Intron, Non-coding; rs232289, Intron, Non-coding;
rs2358666, Intron, Non-coding; rs2363095, Intron, Non-coding; rs2391495, Intron, Non-coding;
rs2395256, Intron, Non-coding; rs241443, Intron, Non-coding; rs2421068, Intron, Non-coding;
rs2421184, Intron, Non-coding; rs2516478, Intron, Non-coding; rs2523512, Intron, Non-coding;
rs2524074, Intron, Non-coding; rs2548655, Intron, Non-coding; rs2594762, Intron, Non-coding;
rs2596487, Intron, Non-coding; rs2597245, Intron, Non-coding; rs2610809, Intron, Non-coding;
rs2621326, Intron, Non-coding; rs2663019, Intron, Non-coding; rs2663030, Intron, Non-coding;
rs2663041, Intron, Non-coding; rs2663052, Intron, Non-coding; rs2671712, Intron, Non-coding;
rs267181, Intron, Non-coding; rs2683314, Intron, Non-coding; rs2687975, Intron, Non-coding;
rs2688791, Intron, Non-coding; rs2707096, Intron, Non-coding; rs2743561, Intron, Non-coding;
rs2752933, Intron, Non-coding; rs2784100, Intron, Non-coding; rs2797909, Intron, Non-coding;
rs2823632, Intron, Non-coding; rs2857103, Intron, Non-coding; rs3018678, Intron, Non-coding;
rs3020212, Intron, Non-coding; rs3024886, Intron, Non-coding; rs3024896, Intron, Non-coding;
rs3024912, Intron, Non-coding; rs308076, Intron, Non-coding; rs3093974, Intron, Non-coding;
rs3094549, Intron, Non-coding; rs3094911, Intron, Non-coding; rs3104369, Intron, Non-coding;
rs3117578, Intron, Non-coding; rs3120699, Intron, Non-coding; rs3130050, Intron, Non-coding;
rs3130284, Intron, Non-coding; rs3132956, Intron, Non-coding; rs3134608, Intron, Non-coding;
rs3134930, Intron, Non-coding; rs3213767, Intron, Non-coding; rs3213829, Intron, Non-coding;
rs334057, Intron, Non-coding; rs340630, Intron, Non-coding; rs34214527, Intron, Non-coding;
rs34241101, Intron, Non-coding; rs34997637, Intron, Non-coding; rs350677, Intron, Non-coding;
rs35265698, Intron, Non-coding; rs374379, Intron, Non-coding; rs3763305, Intron, Non-coding;
rs3763354, Intron, Non-coding; rs3763355, Intron, Non-coding; rs3778504, Intron, Non-coding;
rs3799373, Intron, Non-coding; rs3800755, Intron, Non-coding; rs3806670, Intron, Non-coding;
rs3807306, Intron, Non-coding; rs3817964, Intron, Non-coding; rs3821236, Intron, Non-coding;
rs387608, Intron, Non-coding; rs3982269, Intron, Non-coding; rs411538, Intron, Non-coding;
rs41277398, Intron, Non-coding; rs4148871, Intron, Non-coding; rs4148874, Intron, Non-coding;
rs420436, Intron, Non-coding; rs420463, Intron, Non-coding; rs4248169, Intron, Non-coding;
rs440336, Intron, Non-coding; rs4544722, Intron, Non-coding; rs4563443, Intron, Non-coding;
rs4563976, Intron, Non-coding; rs4610965, Intron, Non-coding; rs4610966, Intron, Non-coding;
rs4647361, Intron, Non-coding; rs4660142, Intron, Non-coding; rs4846443, Intron, Non-coding;
rs4853540, Intron, Non-coding; rs4865868, Intron, Non-coding; rs4896840, Intron, Non-coding;
rs4916334, Intron, Non-coding; rs4944082, Intron, Non-coding; rs4944950, Intron, Non-coding;
rs4959068, Intron, Non-coding; rs5029939, Intron, Non-coding; rs517108, Intron, Non-coding;
rs558059, Intron, Non-coding; rs560514, Intron, Non-coding; rs5914778, Intron, Non-coding;
rs597325, Intron, Non-coding; rs5997729, Intron, Non-coding; rs6057705, Intron, Non-coding;
rs6080708, Intron, Non-coding; rs6118393, Intron, Non-coding; rs61616683, Intron, Non-coding;
rs621007, Intron, Non-coding; rs624354, Intron, Non-coding; rs6446975, Intron, Non-coding;
rs6457281, Intron, Non-coding; rs6484065, Intron, Non-coding; rs652043, Intron, Non-coding;
rs6537101, Intron, Non-coding; rs6553453, Intron, Non-coding; rs6556123, Intron, Non-coding;
rs6570779, Intron, Non-coding; rs670369, Intron, Non-coding; rs6726164, Intron, Non-coding;
rs6756098, Intron, Non-coding; rs6762714, Intron, Non-coding; rs6804441, Intron, Non-coding;
rs6808534, Intron, Non-coding; rs6837395, Intron, Non-coding; rs6838147, Intron, Non-coding;
rs6840437, Intron, Non-coding; rs6867835, Intron, Non-coding; rs6875678, Intron, Non-coding;
rs6882531, Intron, Non-coding; rs6884965, Intron, Non-coding; rs6887062, Intron, Non-coding;
rs6891892, Intron, Non-coding; rs6903608, Intron, Non-coding; rs6905408, Intron, Non-coding;
rs6908631, Intron, Non-coding; rs6911777, Intron, Non-coding; rs6914849, Intron, Non-coding;
rs6918329, Intron, Non-coding; rs6923504, Intron, Non-coding; rs6927023, Intron, Non-coding;
rs6929796, Intron, Non-coding; rs6943711, Intron, Non-coding; rs6946855, Intron, Non-coding;
rs6964720, Intron, Non-coding; rs6964780, Intron, Non-coding; rs6996073, Intron, Non-coding;
rs7011609, Intron, Non-coding; rs7011894, Intron, Non-coding; rs7018416, Intron, Non-coding;
rs7023664, Intron, Non-coding; rs7027037, Intron, Non-coding; rs7039284, Intron, Non-coding;
rs7075349, Intron, Non-coding; rs707937, Intron, Non-coding; rs7094742, Intron, Non-coding;
rs7096295, Intron, Non-coding; rs7108897, Intron, Non-coding; rs7124129, Intron, Non-coding;
rs715299, Intron, Non-coding; rs719150, Intron, Non-coding; rs7282300, Intron, Non-coding;
rs73135369, Intron, Non-coding; rs7341300, Intron, Non-coding; rs7382309, Intron, Non-coding;
rs739389, Intron, Non-coding; rs7453920, Intron, Non-coding; rs7475717, Intron, Non-coding;
rs752637, Intron, Non-coding; rs7549511, Intron, Non-coding; rs7571914, Intron, Non-coding;
rs7574865, Intron, Non-coding; rs7582694, Intron, Non-coding; rs7592105, Intron, Non-coding;
rs7601754, Intron, Non-coding; rs760979, Intron, Non-coding; rs7615524, Intron, Non-coding;
rs7629754, Intron, Non-coding; rs7632524, Intron, Non-coding; rs7643591, Intron, Non-coding;
rs7662915, Intron, Non-coding; rs7671436, Intron, Non-coding; rs7673199, Intron, Non-coding;
rs767725, Intron, Non-coding; rs7690725, Intron, Non-coding; rs7704876, Intron, Non-coding;
rs7712698, Intron, Non-coding; rs7713223, Intron, Non-coding; rs7724542, Intron, Non-coding;
rs7726159, Intron, Non-coding; rs7751015, Intron, Non-coding; rs7766854, Intron, Non-coding;
rs7767493, Intron, Non-coding; rs7769836, Intron, Non-coding; rs7821000, Intron, Non-coding;
rs7836918, Intron, Non-coding; rs7838697, Intron, Non-coding; rs7851794, Intron, Non-coding;
rs7852741, Intron, Non-coding; rs7900165, Intron, Non-coding; rs8118727, Intron, Non-coding;
rs891851, Intron, Non-coding; rs893944, Intron, Non-coding; rs904375, Intron, Non-coding;
rs909253, Intron, Non-coding; rs9257940, Intron, Non-coding; rs9258206, Intron, Non-coding;
rs9258209, Intron, Non-coding; rs9258213, Intron, Non-coding; rs9258218, Intron, Non-coding;
rs9261191, Intron, Non-coding; rs9261869, Intron, Non-coding; rs9264602, Intron, Non-coding;
rs9268199, Intron, Non-coding; rs9268832, Intron, Non-coding; rs9268862, Intron, Non-coding;
rs9268880, Intron, Non-coding; rs9268911, Intron, Non-coding; rs9268977, Intron, Non-coding;
rs9268980, Intron, Non-coding; rs9269043, Intron, Non-coding; rs9275653, Intron, Non-coding;
rs9276427, Intron, Non-coding; rs9349131, Intron, Non-coding; rs9390649, Intron, Non-coding;
rs9393890, Intron, Non-coding; rs9393944, Intron, Non-coding; rs9394052, Intron, Non-coding;
rs9398481, Intron, Non-coding; rs9399137, Intron, Non-coding; rs9450870, Intron, Non-coding;
rs9461455, Intron, Non-coding; rs9463229, Intron, Non-coding; rs9463286, Intron, Non-coding;
rs9468290, Intron, Non-coding; rs9490984, Intron, Non-coding; rs9494883, Intron, Non-coding;
rs9625522, Intron, Non-coding; rs9654550, Intron, Non-coding; rs9657175, Intron, Non-coding;
rs9766788, Intron, Non-coding; rs9809469, Intron, Non-coding; rs9810562, Intron, Non-coding;
rs9810646, Intron, Non-coding; rs981132, Intron, Non-coding; rs981580, Intron, Non-coding;
rs9823236, Intron, Non-coding; rs9833639, Intron, Non-coding; rs9837184, Intron, Non-coding;
rs9844370, Intron, Non-coding; rs9844884, Intron, Non-coding; rs9849219, Intron, Non-coding;
rs9852651, Intron, Non-coding; rs9853999, Intron, Non-coding; rs9854727, Intron, Non-coding;
rs9867081, Intron, Non-coding; rs9871932, Intron, Non-coding; rs9872099, Intron, Non-coding;
rs9876869, Intron, Non-coding; rs9886297, Intron, Non-coding; rs9918368, Intron, Non-coding;
rs995983, Intron, Non-coding; rs9993633, Intron, Non-coding; rs9995753, Intron, Non-coding;
rs2213565, Splice region variant, Non-coding; rs10000263, Intergenic, Non-coding; rs10066427,
Intergenic, Non-coding; rs10119641, Intergenic, Non-coding; rs10216228, Intergenic, Non-coding;
rs11940156, Intergenic, Non-coding; rs11974020, Intergenic, Non-coding; rs13199787, Intergenic,
Non-coding; rs1573648, Intergenic, Non-coding; rs16894948, Intergenic, Non-coding; rs17552904,
Intergenic, Non-coding; rs17837474, Intergenic, Non-coding; rs1799964, Intergenic, Non-coding;
rs1800630, Intergenic, Non-coding; rs2009658, Intergenic, Non-coding; rs2027856, Intergenic, Non-
coding; rs2395175, Intergenic, Non-coding; rs2734573, Intergenic, Non-coding; rs2844480,
Intergenic, Non-coding; rs28490179, Intergenic, Non-coding; rs2859071, Intergenic, Non-coding;
rs3025657, Intergenic, Non-coding; rs3132454, Intergenic, Non-coding; rs3763310, Intergenic, Non-
coding; rs3957146, Intergenic, Non-coding; rs3957148, Intergenic, Non-coding; rs3997854,
Intergenic, Non-coding; rs3998159, Intergenic, Non-coding; rs4590339, Intergenic, Non-coding;
rs4717901, Intergenic, Non-coding; rs4728142, Intergenic, Non-coding; rs4917014, Intergenic, Non-
coding; rs5019296, Intergenic, Non-coding; rs6457009, Intergenic, Non-coding; rs6457644,
Intergenic, Non-coding; rs6902723, Intergenic, Non-coding; rs6903130, Intergenic, Non-coding;
rs6933331, Intergenic, Non-coding; rs7007508, Intergenic, Non-coding; rs7382794, Intergenic, Non-
coding; rs7454108, Intergenic, Non-coding; rs7673539, Intergenic, Non-coding; rs7738261,
Intergenic, Non-coding; rs7755597, Intergenic, Non-coding; rs7771335, Intergenic, Non-coding;
rs7773068, Intergenic, Non-coding; rs7773407, Intergenic, Non-coding; rs7773694, Intergenic, Non-
coding; rs7821172, Intergenic, Non-coding; rs9261817, Intergenic, Non-coding; rs9276291,
Intergenic, Non-coding; rs9276296, Intergenic, Non-coding; rs9276370, Intergenic, Non-coding;
rs9276586, Intergenic, Non-coding; rs9276595, Intergenic, Non-coding; rs9276598, Intergenic, Non-
coding; rs9393737, Intergenic, Non-coding; rs9473668, Intergenic, Non-coding; rs9840829,
Intergenic, Non-coding; rs9847208, Intergenic, Non-coding; rs12022635, Intergenic, Non-coding;
rs10047547, LncRNA, Non-coding RNA; rs1009285, LncRNA, Non-coding RNA; rs10133460,
LncRNA, Non-coding RNA; rs10138985, LncRNA, Non-coding RNA; rs10139092, LncRNA, Non-
coding RNA; rs10144697, LncRNA, Non-coding RNA; rs10149380, LncRNA, Non-coding RNA;
rs1016033, LncRNA, Non-coding RNA; rs10169356, LncRNA, Non-coding RNA; rs10175265,
LncRNA, Non-coding RNA; rs10186017, LncRNA, Non-coding RNA; rs10188239, LncRNA, Non-
coding RNA; rs10192425, LncRNA, Non-coding RNA; rs10196236, LncRNA, Non-coding RNA;
rs10197738, LncRNA, Non-coding RNA; rs10198209, LncRNA, Non-coding RNA; rs10200094,
LncRNA, Non-coding RNA; rs1021483, LncRNA, Non-coding RNA; rs1034378, LncRNA, Non-
coding RNA; rs1034627, LncRNA, Non-coding RNA; rs10400971, LncRNA, Non-coding RNA;
rs10408505, LncRNA, Non-coding RNA; rs10438459, LncRNA, Non-coding RNA; rs10483530,
LncRNA, Non-coding RNA; rs10495579, LncRNA, Non-coding RNA; rs10498294, LncRNA, Non-
coding RNA; rs10507145, LncRNA, Non-coding RNA; rs10744102, LncRNA, Non-coding RNA;
rs10845606, LncRNA, Non-coding RNA; rs10851974, LncRNA, Non-coding RNA; rs10865411,
LncRNA, Non-coding RNA; rs10878490, LncRNA, Non-coding RNA; rs10893872, LncRNA, Non-
coding RNA; rs11044662, LncRNA, Non-coding RNA; rs11044741, LncRNA, Non-coding RNA;
rs11055457, LncRNA, Non-coding RNA; rs11076616, LncRNA, Non-coding RNA; rs11080123,
LncRNA, Non-coding RNA; rs11104219, LncRNA, Non-coding RNA; rs11109762, LncRNA, Non-
coding RNA; rs11150586, LncRNA, Non-coding RNA; rs1115303, LncRNA, Non-coding RNA;
rs11172990, LncRNA, Non-coding RNA; rs1119821, LncRNA, Non-coding RNA; rs11221205,
LncRNA, Non-coding RNA; rs1126307, LncRNA, Non-coding RNA; rs1143834, LncRNA, Non-
coding RNA; rs11613879, LncRNA, Non-coding RNA; rs11619347, LncRNA, Non-coding RNA;
rs11620945, LncRNA, Non-coding RNA; rs11659324, LncRNA, Non-coding RNA; rs11667602,
LncRNA, Non-coding RNA; rs11691629, LncRNA, Non-coding RNA; rs11695567, LncRNA, Non-
coding RNA; rs1170426, LncRNA, Non-coding RNA; rs11835748, LncRNA, Non-coding RNA;
rs11836190, LncRNA, Non-coding RNA; rs1183669, LncRNA, Non-coding RNA; rs11836915,
LncRNA, Non-coding RNA; rs11837888, LncRNA, Non-coding RNA; rs11838634, LncRNA, Non-
coding RNA; rs11849407, LncRNA, Non-coding RNA; rs11851602, LncRNA, Non-coding RNA;
rs11855733, LncRNA, Non-coding RNA; rs11857616, LncRNA, Non-coding RNA; rs11860625,
LncRNA, Non-coding RNA; rs11861392, LncRNA, Non-coding RNA; rs11868642, LncRNA, Non-
coding RNA; rs11870746, LncRNA, Non-coding RNA; rs11872273, LncRNA, Non-coding RNA;
rs11873123, LncRNA, Non-coding RNA; rs11883240, LncRNA, Non-coding RNA; rs1196819,
LncRNA, Non-coding RNA; rs12100517, LncRNA, Non-coding RNA; rs12100709, LncRNA, Non-
coding RNA; rs12104258, LncRNA, Non-coding RNA; rs12184995, LncRNA, Non-coding RNA;
rs12185262, LncRNA, Non-coding RNA; rs12231184, LncRNA, Non-coding RNA; rs12288456,
LncRNA, Non-coding RNA; rs12292417, LncRNA, Non-coding RNA; rs12297193, LncRNA, Non-
coding RNA; rs12300549, LncRNA, Non-coding RNA; rs12300895, LncRNA, Non-coding RNA;
rs12307645, LncRNA, Non-coding RNA; rs12311669, LncRNA, Non-coding RNA; rs12313025,
LncRNA, Non-coding RNA; rs12314874, LncRNA, Non-coding RNA; rs12318192, LncRNA, Non-
coding RNA; rs12318506, LncRNA, Non-coding RNA; rs12321453, LncRNA, Non-coding RNA;
rs12322292, LncRNA, Non-coding RNA; rs12328592, LncRNA, Non-coding RNA; rs1233456,
LncRNA, Non-coding RNA; rs12369002, LncRNA, Non-coding RNA; rs12371784, LncRNA, Non-
coding RNA; rs1241681, LncRNA, Non-coding RNA; rs12430783, LncRNA, Non-coding RNA;
rs1243082, LncRNA, Non-coding RNA; rs12439146, LncRNA, Non-coding RNA; rs12446053,
LncRNA, Non-coding RNA; rs12450679, LncRNA, Non-coding RNA; rs12458730, LncRNA, Non-
coding RNA; rs12459935, LncRNA, Non-coding RNA; rs12467735, LncRNA, Non-coding RNA;
rs12473840, LncRNA, Non-coding RNA; rs1254678, LncRNA, Non-coding RNA; rs12576753,
LncRNA, Non-coding RNA; rs12584300, LncRNA, Non-coding RNA; rs12590429, LncRNA, Non-
coding RNA; rs12599402, LncRNA, Non-coding RNA; rs12609167, LncRNA, Non-coding RNA;
rs12610602, LncRNA, Non-coding RNA; rs12621957, LncRNA, Non-coding RNA; rs12624187,
LncRNA, Non-coding RNA; rs1265877, LncRNA, Non-coding RNA; rs12692358, LncRNA, Non-
coding RNA; rs12716890, LncRNA, Non-coding RNA; rs12786668, LncRNA, Non-coding RNA;
rs12803698, LncRNA, Non-coding RNA; rs12819747, LncRNA, Non-coding RNA; rs1285608,
LncRNA, Non-coding RNA; rs12898094, LncRNA, Non-coding RNA; rs12900339, LncRNA, Non-
coding RNA; rs12918436, LncRNA, Non-coding RNA; rs12944145, LncRNA, Non-coding RNA;
rs12948107, LncRNA, Non-coding RNA; rs12958881, LncRNA, Non-coding RNA; rs12973798,
LncRNA, Non-coding RNA; rs12980791, LncRNA, Non-coding RNA; rs12988883, LncRNA, Non-
coding RNA; rs13022719, LncRNA, Non-coding RNA; rs1330502, LncRNA, Non-coding RNA;
rs1330514, LncRNA, Non-coding RNA; rs13337667, LncRNA, Non-coding RNA; rs13385731,
LncRNA, Non-coding RNA; rs13414726, LncRNA, Non-coding RNA; rs13417676, LncRNA, Non-
coding RNA; rs13419081, LncRNA, Non-coding RNA; rs1349217, LncRNA, Non-coding RNA;
rs1368511, LncRNA, Non-coding RNA; rs1372998, LncRNA, Non-coding RNA; rs1379145,
LncRNA, Non-coding RNA; rs1385374, LncRNA, Non-coding RNA; rs1444623, LncRNA, Non-
coding RNA; rs1481407, LncRNA, Non-coding RNA; rs1496330, LncRNA, Non-coding RNA;
rs1518684, LncRNA, Non-coding RNA; rs1531357, LncRNA, Non-coding RNA; rs1534815,
LncRNA, Non-coding RNA; rs1597045, LncRNA, Non-coding RNA; rs1610555, LncRNA, Non-
coding RNA; rs163230, LncRNA, Non-coding RNA; rs163260, LncRNA, Non-coding RNA;
rs16926286, LncRNA, Non-coding RNA; rs16945627, LncRNA, Non-coding RNA; rs16948798,
LncRNA, Non-coding RNA; rs16953706, LncRNA, Non-coding RNA; rs16954146, LncRNA, Non-
coding RNA; rs16956643, LncRNA, Non-coding RNA; rs16958866, LncRNA, Non-coding RNA;
rs16961779, LncRNA, Non-coding RNA; rs16965104, LncRNA, Non-coding RNA; rs16972959,
LncRNA, Non-coding RNA; rs1698528, LncRNA, Non-coding RNA; rs17035046, LncRNA, Non-
coding RNA; rs17077147, LncRNA, Non-coding RNA; rs17096939, LncRNA, Non-coding RNA;
rs17100147, LncRNA, Non-coding RNA; rs17118971, LncRNA, Non-coding RNA; rs17122173,
LncRNA, Non-coding RNA; rs17190139, LncRNA, Non-coding RNA; rs17213203, LncRNA, Non-
coding RNA; rs17258903, LncRNA, Non-coding RNA; rs17321999, LncRNA, Non-coding RNA;
rs1756299, LncRNA, Non-coding RNA; rs17722716, LncRNA, Non-coding RNA; rs1786813,
LncRNA, Non-coding RNA; rs178770, LncRNA, Non-coding RNA; rs1816589, LncRNA, Non-
coding RNA; rs1882861, LncRNA, Non-coding RNA; rs1950423, LncRNA, Non-coding RNA;
rs1951455, LncRNA, Non-coding RNA; rs1959905, LncRNA, Non-coding RNA; rs196946, LncRNA,
Non-coding RNA; rs1999342, LncRNA, Non-coding RNA; rs2002472, LncRNA, Non-coding RNA;
rs2005775, LncRNA, Non-coding RNA; rs2033862, LncRNA, Non-coding RNA; rs2055270,
LncRNA, Non-coding RNA; rs2056610, LncRNA, Non-coding RNA; rs2106670, LncRNA, Non-
coding RNA; rs2150351, LncRNA, Non-coding RNA; rs2150483, LncRNA, Non-coding RNA;
rs2156697, LncRNA, Non-coding RNA; rs221097, LncRNA, Non-coding RNA; rs2225003, LncRNA,
Non-coding RNA; rs223881, LncRNA, Non-coding RNA; rs2239193, LncRNA, Non-coding RNA;
rs2293320, LncRNA, Non-coding RNA; rs2304426, LncRNA, Non-coding RNA; rs2373114,
LncRNA, Non-coding RNA; rs2398260, LncRNA, Non-coding RNA; rs2415584, LncRNA, Non-
coding RNA; rs2537793, LncRNA, Non-coding RNA; rs261434, LncRNA, Non-coding RNA;
rs265589, LncRNA, Non-coding RNA; rs279148, LncRNA, Non-coding RNA; rs2792102, LncRNA,
Non-coding RNA; rs2806306, LncRNA, Non-coding RNA; rs283989, LncRNA, Non-coding RNA;
rs2840241, LncRNA, Non-coding RNA; rs2978376, LncRNA, Non-coding RNA; rs349955, LncRNA,
Non-coding RNA; rs3782331, LncRNA, Non-coding RNA; rs4255691, LncRNA, Non-coding RNA;
rs4384685, LncRNA, Non-coding RNA; rs4389195, LncRNA, Non-coding RNA; rs4392002,
LncRNA, Non-coding RNA; rs4423343, LncRNA, Non-coding RNA; rs4443040, LncRNA, Non-
coding RNA; rs4545954, LncRNA, Non-coding RNA; rs4622329, LncRNA, Non-coding RNA;
rs4627574, LncRNA, Non-coding RNA; rs4639966, LncRNA, Non-coding RNA; rs4662230,
LncRNA, Non-coding RNA; rs4762432, LncRNA, Non-coding RNA; rs4764430, LncRNA, Non-
coding RNA; rs4770162, LncRNA, Non-coding RNA; rs4777725, LncRNA, Non-coding RNA;
rs4779658, LncRNA, Non-coding RNA; rs4786789, LncRNA, Non-coding RNA; rs4797078,
LncRNA, Non-coding RNA; rs4851415, LncRNA, Non-coding RNA; rs4902213, LncRNA, Non-
coding RNA; rs4906415, LncRNA, Non-coding RNA; rs4913251, LncRNA, Non-coding RNA;
rs4971724, LncRNA, Non-coding RNA; rs607010, LncRNA, Non-coding RNA; rs6494408, LncRNA,
Non-coding RNA; rs6539899, LncRNA, Non-coding RNA; rs6563031, LncRNA, Non-coding RNA;
rs6571811, LncRNA, Non-coding RNA; rs6650482, LncRNA, Non-coding RNA; rs6705628,
LncRNA, Non-coding RNA; rs6721762, LncRNA, Non-coding RNA; rs6727697, LncRNA, Non-
coding RNA; rs7119913, LncRNA, Non-coding RNA; rs7155726, LncRNA, Non-coding RNA;
rs7163683, LncRNA, Non-coding RNA; rs7220877, LncRNA, Non-coding RNA; rs7221079,
LncRNA, Non-coding RNA; rs7230084, LncRNA, Non-coding RNA; rs7306167, LncRNA, Non-
coding RNA; rs7313438, LncRNA, Non-coding RNA; rs7315954, LncRNA, Non-coding RNA;
rs7319464, LncRNA, Non-coding RNA; rs7329174, LncRNA, Non-coding RNA; rs7335023,
LncRNA, Non-coding RNA; rs7494878, LncRNA, Non-coding RNA; rs7579944, LncRNA, Non-
coding RNA; rs766304, LncRNA, Non-coding RNA; rs7934496, LncRNA, Non-coding RNA;
rs7977848, LncRNA, Non-coding RNA; rs7985525, LncRNA, Non-coding RNA; rs8010667,
LncRNA, Non-coding RNA; rs8012094, LncRNA, Non-coding RNA; rs8012564, LncRNA, Non-
coding RNA; rs8012609, LncRNA, Non-coding RNA; rs8036390, LncRNA, Non-coding RNA;
rs8039643, LncRNA, Non-coding RNA; rs8050197, LncRNA, Non-coding RNA; rs8068124,
LncRNA, Non-coding RNA; rs8069771, LncRNA, Non-coding RNA; rs8079112, LncRNA, Non-
coding RNA; rs8098654, LncRNA, Non-coding RNA; rs865952, LncRNA, Non-coding RNA;
rs893298, LncRNA, Non-coding RNA; rs944722, LncRNA, Non-coding RNA; rs9535434, LncRNA,
Non-coding RNA; rs9556348, LncRNA, Non-coding RNA; rs9556447, LncRNA, Non-coding RNA;
rs9564837, LncRNA, Non-coding RNA; rs9591402, LncRNA, Non-coding RNA; rs9600236,
LncRNA, Non-coding RNA; rs9603823, LncRNA, Non-coding RNA; rs9651975, LncRNA, Non-
coding RNA; rs9783661, LncRNA, Non-coding RNA; rs9888674, LncRNA, Non-coding RNA;
rs989092, LncRNA, Non-coding RNA; rs9898036, LncRNA, Non-coding RNA; rs9916655, LncRNA,
Non-coding RNA; rs9921200, LncRNA, Non-coding RNA; rs9923791, LncRNA, Non-coding RNA;
rs993504, LncRNA, Non-coding RNA; rs9946641, LncRNA, Non-coding RNA; rs9956715, LncRNA,
Non-coding RNA; rs9959662, LncRNA, Non-coding RNA; rs9971728, LncRNA, Non-coding RNA;
rs12151634, CTCF, Regulatory; rs17576984, CTCF, Regulatory; rs6434666, CTCF, Regulatory;
rs7439917, CTCF, Regulatory; rs844648, CTCF, Regulatory; rs9275224, CTCF, Regulatory;
rs11609867, Enhancer, Regulatory; rs17089381, Enhancer, Regulatory; rs6464534, Enhancer,
Regulatory; rs73366469, Enhancer, Regulatory; rs12244316, OCR, Regulatory; rs12643514, OCR,
Regulatory; rs12991297, OCR, Regulatory; rs1355567, OCR, Regulatory; rs6598270, OCR,
Regulatory; rs9587391, OCR, Regulatory; rs10239000, PFR, Regulatory; rs10957923, PFR,
Regulatory; rs11896207, PFR, Regulatory; rs12480103, PFR, Regulatory; rs12593568, PFR,
Regulatory; rs12662462, PFR, Regulatory; rs17721983, PFR, Regulatory; rs1885889, PFR,
Regulatory; rs2395309, PFR, Regulatory; rs376877, PFR, Regulatory; rs422544, PFR, Regulatory;
rs4396974, PFR, Regulatory; rs4916319, PFR, Regulatory; rs4972960, PFR, Regulatory; rs6479787,
PFR, Regulatory; rs7936180, PFR, Regulatory; rs9271348, PFR, Regulatory; rs9493230, PFR,
Regulatory; rs964570, PFR, Regulatory; rs12290644, TFBS, Regulatory; rs9275374, TFBS, Regulatory;

TABLE 19
AsA validation GWAS SNP-predicted genes. Listed by: Gene, Source, Ancestry.

1-Mar, P-Gene, AsA; 9-Sep, P-Gene, AsA; 44075, E-Gene, AsA; 7SK, P-Gene, AsA; A1BG, E-Gene,
AsA; A1BG-AS1, E-Gene, AsA; ABCA2, E-Gene, AsA; ABCB5, P-Gene, AsA; ABCD2, E-Gene,
AsA; ABCG4, T-Gene, AsA; ABHD11-AS1, E-Gene, AsA; ABHD12, E-Gene, AsA; ABHD17B, E-
Gene, AsA; ABLIM1, P-Gene, AsA; ABO, E-Gene, AsA; ABT1, P-Gene, AsA; AC003956.1, E-
Gene, AsA; AC004253.1, E-Gene, AsA; AC004593.3, T-Gene, AsA; AC004869.3, P-Gene, AsA;
AC004982.2, E-Gene, AsA; AC005077.4, E-Gene, AsA; AC005307.1, P-Gene, AsA; AC005332.1, E-
Gene, AsA; AC005332.3, E-Gene, AsA; AC005332.5, E-Gene, AsA; AC005532.1, E-Gene, AsA;
AC006001.2, E-Gene, AsA; AC006001.3, E-Gene, AsA; AC006007.1, E-Gene, AsA; AC006007.1, P-
Gene, AsA; AC006019.3, P-Gene, AsA; AC006322.1, P-Gene, AsA; AC006372.3, E-Gene, AsA;
AC006372.4, P-Gene, AsA; AC006926.1, P-Gene, AsA; AC007038.2, E-Gene, AsA; AC007255.1, E-
Gene, AsA; AC007255.7, P-Gene, AsA; AC007285.7, T-Gene, AsA; AC007319.1, P-Gene, AsA;
AC007364.1, P-Gene, AsA; AC007381.1, E-Gene, AsA; AC007563.5, P-Gene, AsA; AC007682.1, P-
Gene, AsA; AC007743.1, E-Gene, AsA; AC008014.1, E-Gene, AsA; AC008267.2, E-Gene, AsA;
AC008267.3, E-Gene, AsA; AC008267.6, E-Gene, AsA; AC008443.6, E-Gene, AsA; AC008610.1, E-
Gene, AsA; AC008703.1, P-Gene, AsA; AC009095.1, E-Gene, AsA; AC009133.20, P-Gene, AsA;
AC009542.1, E-Gene, AsA; AC009695.1, P-Gene, AsA; AC009754.1, E-Gene, AsA; AC009974.1, E-
Gene, AsA; AC010186.2, E-Gene, AsA; AC010207.1, E-Gene, AsA; AC010329.1, E-Gene, AsA;
AC010395.1, E-Gene, AsA; AC010615.1, E-Gene, AsA; AC010615.2, E-Gene, AsA; AC010739.1, P-
Gene, AsA; AC011288.2, P-Gene, AsA; AC011306.1, E-Gene, AsA; AC011747.3, P-Gene, AsA;
AC012313.2, E-Gene, AsA; AC012313.5, E-Gene, AsA; AC012313.8, E-Gene, AsA; AC012510.1, E-
Gene, AsA; AC012645.1, E-Gene, AsA; AC012645.2, E-Gene, AsA; AC013461.1, P-Gene, AsA;
AC013468.1, E-Gene, AsA; AC015853.2, E-Gene, AsA; AC016768.1, P-Gene, AsA; AC016970.1, P-
Gene, AsA; AC016993.1, E-Gene, AsA; AC017067.1, E-Gene, AsA; AC018638.5, E-Gene, AsA;
AC018731.3, P-Gene, AsA; AC018737.3, P-Gene, AsA; AC018799.1, P-Gene, AsA; AC018804.1, E-
Gene, AsA; AC020743.2, T-Gene, AsA; AC020743.3, P-Gene, AsA; AC020743.4, P-Gene, AsA;
AC021148.1, E-Gene, AsA; AC021785.1, E-Gene, AsA; AC021887.1, P-Gene, AsA; AC022075.1, E-
Gene, AsA; AC022075.3, E-Gene, AsA; AC022239.1, E-Gene, AsA; AC022239.2, E-Gene, AsA;
AC022239.3, E-Gene, AsA; AC022239.4, E-Gene, AsA; AC022596.1, E-Gene, AsA; AC022819.1, E-
Gene, AsA; AC023043.3, E-Gene, AsA; AC023794.2, E-Gene, AsA; AC023794.5, E-Gene, AsA;
AC023906.4, E-Gene, AsA; AC024597.1, E-Gene, AsA; AC026150.1, E-Gene, AsA; AC026150.2, E-
Gene, AsA; AC026150.3, E-Gene, AsA; AC027644.1, E-Gene, AsA; AC027644.3, E-Gene, AsA;
AC035139.1, E-Gene, AsA; AC036108.2, E-Gene, AsA; AC036108.3, E-Gene, AsA; AC062028.1, P-
Gene, AsA; AC067945.3, E-Gene, AsA; AC067945.4, P-Gene, AsA; AC068305.3, E-Gene, AsA;
AC068533.2, E-Gene, AsA; AC068535.2, P-Gene, AsA; AC069146.2, P-Gene, AsA; AC069243.1, P-
Gene, AsA; AC069285.1, E-Gene, AsA; AC073218.1, P-Gene, AsA; AC073236.3, P-Gene, AsA;
AC073263.1, T-Gene, AsA; AC073288.1, E-Gene, AsA; AC073288.2, E-Gene, AsA; AC073335.1, E-
Gene, AsA; AC073335.2, E-Gene, AsA; AC073343.1, E-Gene, AsA; AC073343.2, E-Gene, AsA;
AC073349.1, E-Gene, AsA; AC074093.1, P-Gene, AsA; AC074391.2, P-Gene, AsA; AC078795.1, E-
Gene, AsA; AC078828.1, P-Gene, AsA; AC079140.1, P-Gene, AsA; AC080013.1, E-Gene, AsA;
AC080013.3, E-Gene, AsA; AC080013.4, E-Gene, AsA; AC080094.1, P-Gene, AsA; AC083862.2, E-
Gene, AsA; AC083949.1, E-Gene, AsA; AC084125.2, E-Gene, AsA; AC084193.1, P-Gene, AsA;
AC087203.1, E-Gene, AsA; AC087203.2, E-Gene, AsA; AC087481.1, E-Gene, AsA; AC087481.2, E-
Gene, AsA; AC087481.3, E-Gene, AsA; AC089998.1, E-Gene, AsA; AC090616.2, E-Gene, AsA;
AC090616.3, E-Gene, AsA; AC090825.1, E-Gene, AsA; AC090825.2, P-Gene, AsA; AC091042.1, P-
Gene, AsA; AC091057.1, E-Gene, AsA; AC091057.2, E-Gene, AsA; AC091057.3, E-Gene, AsA;
AC091153.3, E-Gene, AsA; AC092364.2, E-Gene, AsA; AC092364.4, P-Gene, AsA; AC092611.2, E-
Gene, AsA; AC092687.3, E-Gene, AsA; AC092718.2, E-Gene, AsA; AC092718.4, E-Gene, AsA;
AC092962.1, P-Gene, AsA; AC093171.1, P-Gene, AsA; AC093297.2, E-Gene, AsA; AC093582.1, E-
Gene, AsA; AC093843.1, P-Gene, AsA; AC093865.1, P-Gene, AsA; AC095055.1, E-Gene, AsA;
AC096559.1, P-Gene, AsA; AC096751.1, E-Gene, AsA; AC096763.1, P-Gene, AsA; AC098679.2, E-
Gene, AsA; AC098850.2, E-Gene, AsA; AC098850.3, E-Gene, AsA; AC099754.1, P-Gene, AsA;
AC103881.1, P-Gene, AsA; AC104261.1, E-Gene, AsA; AC104441.1, P-Gene, AsA; AC104574.2, E-
Gene, AsA; AC104667.2, E-Gene, AsA; AC104819.3, E-Gene, AsA; AC104837.1, P-Gene, AsA;
AC104964.3, E-Gene, AsA; AC105252.1, P-Gene, AsA; AC105402.4, T-Gene, AsA; AC106795.2, E-
Gene, AsA; AC107375.1, E-Gene, AsA; AC108739.1, P-Gene, AsA; AC108866.1, E-Gene, AsA;
AC110086.1, P-Gene, AsA; AC112198.2, P-Gene, AsA; AC112487.1, E-Gene, AsA; AC113195.1, P-
Gene, AsA; AC114788.2, P-Gene, AsA; AC115621.1, E-Gene, AsA; AC116158.1, E-Gene, AsA;
AC116407.1, E-Gene, AsA; AC117401.1, P-Gene, AsA; AC123767.1, E-Gene, AsA; AC123912.1, E-
Gene, AsA; AC123912.4, E-Gene, AsA; AC124014.1, T-Gene, AsA; AC126323.1, E-Gene, AsA;
AC126323.6, E-Gene, AsA; AC127502.1, E-Gene, AsA; AC127502.2, E-Gene, AsA; AC128688.2, E-
Gene, AsA; AC130352.1, E-Gene, AsA; AC132803.1, P-Gene, AsA; AC133633.1, P-Gene, AsA;
AC135803.1, E-Gene, AsA; AC136628.3, E-Gene, AsA; AC136632.1, E-Gene, AsA; AC138207.6, E-
Gene, AsA; AC139426.1, E-Gene, AsA; AC139453.1, P-Gene, AsA; AC140125.2, E-Gene, AsA;
AC244034.2, E-Gene, AsA; ACADVL, C-Gene, AsA; ACADVL, P-Gene, AsA; ACIN1, T-Gene,
AsA; ACPL2, P-Gene, AsA; ACSL1, P-Gene, AsA; ACSM1, E-Gene, AsA; ACSM3, E-Gene, AsA;
ACSM3, P-Gene, AsA; ACSM5, E-Gene, AsA; ACTG2, T-Gene, AsA; ACTL8, P-Gene, AsA;
ADAM18, E-Gene, AsA; ADAM18, P-Gene, AsA; ADAM23, E-Gene, AsA; ADAM3A, E-Gene,
AsA; ADAM5, E-Gene, AsA; ADAMTS3, P-Gene, AsA; ADCY10, P-Gene, AsA; ADCY3, E-Gene,
AsA; ADGRB3, E-Gene, AsA; ADO, E-Gene, AsA; AE000661.37, P-Gene, AsA; AEBP2, E-Gene,
AsA; AEBP2, P-Gene, AsA; AF117829.1, E-Gene, AsA; AF131215.5, E-Gene, AsA; AF131215.6, E-
Gene, AsA; AF131216.3, E-Gene, AsA; AF131217.1, P-Gene, AsA; AF165147.1, E-Gene, AsA;
AF201337.1, E-Gene, AsA; AFF1, P-Gene, AsA; AGER, T-Gene, AsA; AGFG1, E-Gene, AsA;
AGFG1, P-Gene, AsA; AGO1, E-Gene, AsA; AGO3, E-Gene, AsA; AGO4, E-Gene, AsA; AGPAT1,
P-Gene, AsA; AGPAT1, T-Gene, AsA; AHCY, P-Gene, AsA; AHI1, E-Gene, AsA; AHI1, T-Gene,
AsA; AHNAK2, E-Gene, AsA; AHNAK2, P-Gene, AsA; AIF1, P-Gene, AsA; AJ006995.3, P-Gene,
AsA; AKAP2, E-Gene, AsA; AL035604.1, E-Gene, AsA; AL049552.1, E-Gene, AsA; AL049875.1,
E-Gene, AsA; AL078587.1, E-Gene, AsA; AL096803.2, E-Gene, AsA; AL109917.1, E-Gene, AsA;
AL118511.1, E-Gene, AsA; AL121603.2, E-Gene, AsA; AL121658.1, E-Gene, AsA; AL121821.1, E-
Gene, AsA; AL121932.1, P-Gene, AsA; AL121974.1, E-Gene, AsA; AL121983.2, E-Gene, AsA;
AL132639.2, E-Gene, AsA; AL132780.1, E-Gene, AsA; AL136311.1, E-Gene, AsA; AL138498.1, P-
Gene, AsA; AL138787.2, E-Gene, AsA; AL139353.1, E-Gene, AsA; AL139815.1, T-Gene, AsA;
AL160286.2, E-Gene, AsA; AL160286.3, E-Gene, AsA; AL161912.1, E-Gene, AsA; AL162231.2, E-
Gene, AsA; AL162759.1, P-Gene, AsA; AL353593.1, E-Gene, AsA; AL353593.2, E-Gene, AsA;
AL353593.3, E-Gene, AsA; AL353596.1, E-Gene, AsA; AL353622.1, E-Gene, AsA; AL356154.1, P-
Gene, AsA; AL356258.1, E-Gene, AsA; AL356489.2, E-Gene, AsA; AL357060.1, T-Gene, AsA;
AL357060.2, E-Gene, AsA; AL359532.1, E-Gene, AsA; AL359853.1, E-Gene, AsA; AL359853.3, E-
Gene, AsA; AL390198.1, E-Gene, AsA; AL390198.2, E-Gene, AsA; AL391538.1, P-Gene, AsA;
AL391807.1, E-Gene, AsA; AL450124.1, E-Gene, AsA; AL450384.2, E-Gene, AsA; AL450996.1, E-
Gene, AsA; AL512328.1, E-Gene, AsA; AL583784.1, P-Gene, AsA; AL583810.2, E-Gene, AsA;
AL589745.1, E-Gene, AsA; AL591623.1, E-Gene, AsA; AL591848.3, E-Gene, AsA; AL645933.2, E-
Gene, AsA; AL662844.4, E-Gene, AsA; AL669914.1, P-Gene, AsA; AL671883.1, E-Gene, AsA;
AL671883.1, P-Gene, AsA; AL671883.2, E-Gene, AsA; AL731892.1, E-Gene, AsA; ALCAM, P-
Gene, AsA; ALDH1A1, P-Gene, AsA; ALDH8A1, E-Gene, AsA; ALDH9A1, E-Gene, AsA;
ALG1L11P, E-Gene, AsA; ALOX15, E-Gene, AsA; AMD1, T-Gene, AsA; AMZ2, E-Gene, AsA;
ANAPC13, E-Gene, AsA; ANAPC13, P-Gene, AsA; ANAPC7, E-Gene, AsA; ANGPT2, P-Gene,
AsA; ANGPTL3, P-Gene, AsA; ANK3, P-Gene, AsA; ANKAR, E-Gene, AsA; ANKEF1, E-Gene,
AsA; ANKHD1, P-Gene, AsA; ANKRA2, E-Gene, AsA; ANKRA2, P-Gene, AsA; ANKRD13C, P-
Gene, AsA; ANKRD45, E-Gene, AsA; ANKRD61, E-Gene, AsA; ANKRD62, E-Gene, AsA;
ANKRD62, P-Gene, AsA; ANKRD7, E-Gene, AsA; ANKRD7, P-Gene, AsA; ANKS1B, P-Gene,
AsA; ANXA10, E-Gene, AsA; ANXA10, P-Gene, AsA; AP000255.1, E-Gene, AsA; AP000473.5, P-
Gene, AsA; AP000722.1, E-Gene, AsA; AP000766.1, E-Gene, AsA; AP000769.1, E-Gene, AsA;
AP000797.4, P-Gene, AsA; AP001001.1, E-Gene, AsA; AP001085.1, E-Gene, AsA; AP001189.1, E-
Gene, AsA; AP001189.3, E-Gene, AsA; AP001453.2, E-Gene, AsA; AP001542.1, E-Gene, AsA;
AP001542.3, E-Gene, AsA; AP001972.1, E-Gene, AsA; AP001992.1, E-Gene, AsA; AP001999.1, E-
Gene, AsA; AP001999.3, E-Gene, AsA; AP002004.1, E-Gene, AsA; AP002360.4, E-Gene, AsA;
AP002414.2, E-Gene, AsA; AP002414.3, E-Gene, AsA; AP002954.3, T-Gene, AsA; AP003068.2, E-
Gene, AsA; AP003072.2, E-Gene, AsA; AP003730.2, E-Gene, AsA; AP005233.2, E-Gene, AsA;
AP005264.4, E-Gene, AsA; AP005264.5, E-Gene, AsA; AP005329.1, E-Gene, AsA; AP005329.2, E-
Gene, AsA; AP5B1, E-Gene, AsA; AP5B1, P-Gene, AsA; AP5B1, T-Gene, AsA; APH1B, E-Gene,
AsA; APIP, E-Gene, AsA; APOL3, E-Gene, AsA; APOL3, P-Gene, AsA; APOL4, E-Gene, AsA;
APOLD1, E-Gene, AsA; APOM, E-Gene, AsA; APOM, T-Gene, AsA; APPL1, E-Gene, AsA; AQP1,
P-Gene, AsA; AQP4-AS1, E-Gene, AsA; ARAP1, E-Gene, AsA; ARAP1, T-Gene, AsA; ARAP1-
AS1, T-Gene, AsA; ARAP1-AS2, T-Gene, AsA; ARCN1, T-Gene, AsA; AREG, P-Gene, AsA;
ARF4, E-Gene, AsA; ARF4-AS1, E-Gene, AsA; ARHGAP10, P-Gene, AsA; ARHGAP11B, E-Gene,
AsA; ARHGAP42, E-Gene, AsA; ARHGEF17, T-Gene, AsA; ARHGEF28, E-Gene, AsA;
ARHGEF4, E-Gene, AsA; ARL6IP6, P-Gene, AsA; ARMC3, P-Gene, AsA; ARMC3, T-Gene, AsA;
ARPC3, E-Gene, AsA; ARPC4-TTLL3, P-Gene, AsA; ARPP19, P-Gene, AsA; ARRB1, E-Gene,
AsA; ARRB2, E-Gene, AsA; ARRB2, P-Gene, AsA; ARSB, P-Gene, AsA; ARSG, E-Gene, AsA;
ASAH1, E-Gene, AsA; ASB14, E-Gene, AsA; ASL, E-Gene, AsA; ASNSD1, E-Gene, AsA; ASPG,
E-Gene, AsA; ASPG, P-Gene, AsA; ASPG, T-Gene, AsA; ASTN2, P-Gene, AsA; ATF6B, T-Gene,
AsA; ATG16L2, C-Gene, AsA; ATG16L2, P-Gene, AsA; ATG16L2, T-Gene, AsA; ATG5, E-Gene,
AsA; ATG5, P-Gene, AsA; ATMIN, E-Gene, AsA; ATP10A, P-Gene, AsA; ATP2A2, E-Gene, AsA;
ATP5L, T-Gene, AsA; ATP6V0B, E-Gene, AsA; ATP6V1C2, E-Gene, AsA; ATP6V1D, E-Gene,
AsA; ATP6V1G2, E-Gene, AsA; ATP6V1G2, P-Gene, AsA; ATP6V1G2, T-Gene, AsA; ATP6V1G2-
DDX39B, P-Gene, AsA; ATP6V1G2-DDX39B, T-Gene, AsA; ATPBD4, P-Gene, AsA; ATRNL1, P-
Gene, AsA; ATXN1, P-Gene, AsA; AUP1, T-Gene, AsA; AXDND1, E-Gene, AsA; B3GAT1, E-
Gene, AsA; B4GALNT2, P-Gene, AsA; BACH2, P-Gene, AsA; BAG2, E-Gene, AsA; BAG5, E-
Gene, AsA; BAG6, E-Gene, AsA; BAG6, P-Gene, AsA; BCHE, E-Gene, AsA; BCHE, P-Gene, AsA;
BCL11A, P-Gene, AsA; BCL9L, T-Gene, AsA; BCLAF1, T-Gene, AsA; BCMO1, T-Gene, AsA;
BCS1L, E-Gene, AsA; BEND6, E-Gene, AsA; BEST4, E-Gene, AsA; BLK, E-Gene, AsA; BLK, P-
Gene, AsA; BLMH, P-Gene, AsA; BMI1, P-Gene, AsA; BMP5, P-Gene, AsA; BMP6, P-Gene, AsA;
BMPR1B, E-Gene, AsA; BMPR1B, P-Gene, AsA; BNIPL, E-Gene, AsA; BOLA2, E-Gene, AsA;
BOLA3, T-Gene, AsA; BOLA3-AS1, T-Gene, AsA; BPIFB6, P-Gene, AsA; BRD2, T-Gene, AsA;
BRD2-IT1, P-Gene, AsA; BTBD19, E-Gene, AsA; BTNL2, P-Gene, AsA; BX284668.6, E-Gene,
AsA; BZW1P2, E-Gene, AsA; C10orf112, P-Gene, AsA; C10orf115, T-Gene, AsA; C10orf90, P-
Gene, AsA; C11orf53, P-Gene, AsA; C12orf26, P-Gene, AsA; C12orf4, P-Gene, AsA; C12orf40, P-
Gene, AsA; C12orf55, P-Gene, AsA; C12orf63, T-Gene, AsA; C12orf76, E-Gene, AsA; C13orf44-
AS1, P-Gene, AsA; C14orf119, T-Gene, AsA; C14orf164, P-Gene, AsA; C14orf180, E-Gene, AsA;
C14orf2, E-Gene, AsA; C14orf2, T-Gene, AsA; C14orf93, T-Gene, AsA; C15orf29, P-Gene, AsA;
C17orf80, E-Gene, AsA; C1GALT1, E-Gene, AsA; C1orf147, E-Gene, AsA; C1orf198, E-Gene, AsA;
C1orf216, E-Gene, AsA; C1orf216, P-Gene, AsA; C1orf228, E-Gene, AsA; C2, E-Gene, AsA;
C2CD3, E-Gene, AsA; C2orf65, T-Gene, AsA; C2orf78, T-Gene, AsA; C2orf81, T-Gene, AsA;
C3orf15, P-Gene, AsA; C3orf52, E-Gene, AsA; C4A, E-Gene, AsA; C4A, T-Gene, AsA; C4B, E-
Gene, AsA; C4orf27, P-Gene, AsA; C4orf46, P-Gene, AsA; C6orf10, P-Gene, AsA; C6orf10, T-Gene,
AsA; C6orf100, T-Gene, AsA; C6orf106, E-Gene, AsA; C6orf118, P-Gene, AsA; C6orf136, T-Gene,
AsA; C6orf15, T-Gene, AsA; C6orf163, E-Gene, AsA; C6orf25, P-Gene, AsA; C6orf26, T-Gene,
AsA; C7orf26, E-Gene, AsA; C7orf49, E-Gene, AsA; C7orf72, T-Gene, AsA; C8orf49, E-Gene, AsA;
C8orf59, E-Gene, AsA; C9orf152, E-Gene, AsA; C9orf152, P-Gene, AsA; C9orf85, E-Gene, AsA;
CA13, E-Gene, AsA; CAAP1, P-Gene, AsA; CACNA2D3, P-Gene, AsA; CALD1, P-Gene, AsA;
CALML6, E-Gene, AsA; CALU, T-Gene, AsA; CAMK2G, P-Gene, AsA; CAPN9, E-Gene, AsA;
CAPS2, P-Gene, AsA; CASC10, E-Gene, AsA; CASP1, E-Gene, AsA; CATSPERB, P-Gene, AsA;
CBFA2T3, P-Gene, AsA; CBLB, E-Gene, AsA; CBX5, E-Gene, AsA; CCDC115, E-Gene, AsA;
CCDC117, E-Gene, AsA; CCDC129, E-Gene, AsA; CCDC129, P-Gene, AsA; CCDC136, T-Gene,
AsA; CCDC14, E-Gene, AsA; CCDC144A, E-Gene, AsA; CCDC148-AS1, E-Gene, AsA; CCDC183-
AS1, E-Gene, AsA; CCDC196, E-Gene, AsA; CCDC58P3, E-Gene, AsA; CCDC74B, E-Gene, AsA;
CCDC84, T-Gene, AsA; CCDC85A, E-Gene, AsA; CCDC85A, P-Gene, AsA; CCDC86, E-Gene,
AsA; CCHCR1, E-Gene, AsA; CCHCR1, T-Gene, AsA; CCL17, E-Gene, AsA; CCL20, P-Gene,
AsA; CCL22, P-Gene, AsA; CCND1, E-Gene, AsA; CCNE2, E-Gene, AsA; CCNE2, P-Gene, AsA;
CCR1, E-Gene, AsA; CCR3, E-Gene, AsA; CCR5, E-Gene, AsA; CCR9, E-Gene, AsA; CCRL2, E-
Gene, AsA; CCT6A, P-Gene, AsA; CCT6P3, E-Gene, AsA; CCT8, E-Gene, AsA; CCZ1, E-Gene,
AsA; CCZ1B, E-Gene, AsA; CD226, E-Gene, AsA; CD226, P-Gene, AsA; CD274, E-Gene, AsA;
CD2BP2, E-Gene, AsA; CD38, E-Gene, AsA; CD38, P-Gene, AsA; CD44, P-Gene, AsA; CD46, P-
Gene, AsA; CD47, E-Gene, AsA; CD5, E-Gene, AsA; CD80, P-Gene, AsA; CDC42SE1, E-Gene,
AsA; CDC7, P-Gene, AsA; CDCA7, E-Gene, AsA; CDH1, E-Gene, AsA; CDH18, P-Gene, AsA;
CDH20, P-Gene, AsA; CDH24, T-Gene, AsA; CDH3, E-Gene, AsA; CDH4, P-Gene, AsA; CDKL1,
P-Gene, AsA; CDKL3, T-Gene, AsA; CDSN, T-Gene, AsA; CDYL2, T-Gene, AsA; CEBPE, T-Gene,
AsA; CELF4, P-Gene, AsA; CELSR1, P-Gene, AsA; CENPBD1IP1, E-Gene, AsA; CENPL, E-Gene,
AsA; CENPN, E-Gene, AsA; CENPN, T-Gene, AsA; CENPQ, E-Gene, AsA; CEP170B, E-Gene,
AsA; CEP44, E-Gene, AsA; CEP63, E-Gene, AsA; CEP97, E-Gene, AsA; CERKL, P-Gene, AsA;
CERNA1, E-Gene, AsA; CERS2, E-Gene, AsA; CFAP74, E-Gene, AsA; CFH, E-Gene, AsA; CFH,
P-Gene, AsA; CFHR1, E-Gene, AsA; CFHR3, E-Gene, AsA; CHEK2, E-Gene, AsA; CHN2, E-Gene,
AsA; CHN2, T-Gene, AsA; CHORDC1, P-Gene, AsA; CHRDL2, E-Gene, AsA; CHRFAM7A, E-
Gene, AsA; CHRNA7, E-Gene, AsA; CHST9-AS1, P-Gene, AsA; CHTOP, E-Gene, AsA; CIAPIN1,
E-Gene, AsA; CICP24, E-Gene, AsA; CIR1, P-Gene, AsA; CITED2, T-Gene, AsA; CLBA1, E-Gene,
AsA; CLDN11, E-Gene, AsA; CLEC16A, P-Gene, AsA; CLGN, E-Gene, AsA; CLIC1, E-Gene, AsA;
CLIC1, T-Gene, AsA; CLMAT3, E-Gene, AsA; CLSPN, E-Gene, AsA; CLYBL, P-Gene, AsA;
CMC2, E-Gene, AsA; CMC2, T-Gene, AsA; CMYA5, P-Gene, AsA; CNBD1, P-Gene, AsA; CNOT9,
E-Gene, AsA; CNST, E-Gene, AsA; CNTN4, P-Gene, AsA; CNTN5, P-Gene, AsA; CNTN6, P-Gene,
AsA; CNTNAP2, E-Gene, AsA; CNTNAP5, P-Gene, AsA; COA7, E-Gene, AsA; COL11A2, T-Gene,
AsA; COL28A1, P-Gene, AsA; COL8A1, E-Gene, AsA; COL8A2, E-Gene, AsA; COLQ, E-Gene,
AsA; COPRS, E-Gene, AsA; COQ9, E-Gene, AsA; CORO1A, E-Gene, AsA; COX6B1, E-Gene,
AsA; COX6B1, P-Gene, AsA; COX7A2L, E-Gene, AsA; CPA6, P-Gene, AsA; CPB2, P-Gene, AsA;
CPB2-AS1, E-Gene, AsA; CPO, E-Gene, AsA; CPOX, T-Gene, AsA; CPSF2, P-Gene, AsA; CPVL,
T-Gene, AsA; CRCP, E-Gene, AsA; CREB3L4, E-Gene, AsA; CRISP2, E-Gene, AsA; CROT, P-
Gene, AsA; CRTC1, P-Gene, AsA; CRYBG3, T-Gene, AsA; CRYZ, E-Gene, AsA; CRYZ, P-Gene,
AsA; CSF1R, E-Gene, AsA; CSGALNACT1, E-Gene, AsA; CSMD1, P-Gene, AsA; CSNK1G3, P-
Gene, AsA; CSNK2B, P-Gene, AsA; CSRNP3, E-Gene, AsA; CSTA, P-Gene, AsA; CTAGE6, E-
Gene, AsA; CTB-12O2.1, P-Gene, AsA; CTB-57H20.1, P-Gene, AsA; CTBS, E-Gene, AsA; CTC-
254B4.1, P-Gene, AsA; CTC-564N23.3, P-Gene, AsA; CTD-2010I22.2, P-Gene, AsA; CTD-
2012I17.1, P-Gene, AsA; CTD-2023M8.1, P-Gene, AsA; CTD-2151A2.1, P-Gene, AsA; CTD-
2201G16.1, T-Gene, AsA; CTD-2232E5.2, P-Gene, AsA; CTD-2281M20.1, P-Gene, AsA; CTD-
2307P3.1, P-Gene, AsA; CTD-2308B18.3, P-Gene, AsA; CTD-2335K5.1, P-Gene, AsA; CTD-
2336H13.2, P-Gene, AsA; CTD-2341M24.1, P-Gene, AsA; CTD-2540C19.1, P-Gene, AsA;
CTNNA3, P-Gene, AsA; CTSB, E-Gene, AsA; CTSO, E-Gene, AsA; CTSW, E-Gene, AsA; CUL5, P-
Gene, AsA; CWF19L2, E-Gene, AsA; CX3CL1, E-Gene, AsA; CXCL16, E-Gene, AsA; CXCR5, T-
Gene, AsA; CXCR6, E-Gene, AsA; CYB5B, E-Gene, AsA; CYB5B, P-Gene, AsA; CYP21A1P, E-
Gene, AsA; CYP21A2, E-Gene, AsA; CYP24A1, E-Gene, AsA; CYP24A1, P-Gene, AsA; CYP27A1,
E-Gene, AsA; CYP27B1, E-Gene, AsA; CYP39A1, E-Gene, AsA; CYP39A1, P-Gene, AsA;
CYP4A11, E-Gene, AsA; CYP4A22, E-Gene, AsA; CYP4A22-AS1, E-Gene, AsA; CYP4B1, E-Gene,
AsA; CYP4X1, E-Gene, AsA; CYP4X1, P-Gene, AsA; CYP4Z1, E-Gene, AsA; CYP4Z1, P-Gene,
AsA; CYTH3, E-Gene, AsA; DAAM1, E-Gene, AsA; DAAM1, P-Gene, AsA; DAB2, P-Gene, AsA;
DACH1, P-Gene, AsA; DAGLA, E-Gene, AsA; DAGLA, P-Gene, AsA; DAND5, E-Gene, AsA;
DAND5, P-Gene, AsA; DAPK2, E-Gene, AsA; DAQB-12N14.5, P-Gene, AsA; DBIL5P, E-Gene,
AsA; DCAF12, P-Gene, AsA; DCAF4L1, E-Gene, AsA; DCAF6, E-Gene, AsA; DCK, E-Gene, AsA;
DCK, P-Gene, AsA; DCLK2, E-Gene, AsA; DCLRE1A, P-Gene, AsA; DCT, E-Gene, AsA; DCTN1,
T-Gene, AsA; DCUN1D3, P-Gene, AsA; DDR1, P-Gene, AsA; DDX10P1, E-Gene, AsA; DDX39B,
C-Gene, AsA; DDX39B, T-Gene, AsA; DDX39B-AS1, T-Gene, AsA; DDX46, T-Gene, AsA; DDX6,
T-Gene, AsA; DECR1, T-Gene, AsA; DEF6, E-Gene, AsA; DEF6, P-Gene, AsA; DEFB131E, E-
Gene, AsA; DEFB134, E-Gene, AsA; DENND3, P-Gene, AsA; DENND6A, E-Gene, AsA;
DENND6A-AS1, E-Gene, AsA; DERA, P-Gene, AsA; DGUOK, T-Gene, AsA; DGUOK-AS1, P-
Gene, AsA; DHRS4-AS1, E-Gene, AsA; DHX15, E-Gene, AsA; DHX15, P-Gene, AsA; DIS3L2, P-
Gene, AsA; DKFZP434K028, E-Gene, AsA; DKK2, P-Gene, AsA; DLD, T-Gene, AsA; DLEU1, P-
Gene, AsA; DLG2, P-Gene, AsA; DLGAP1, P-Gene, AsA; DLGAP3, E-Gene, AsA; DMRTA1, E-
Gene, AsA; DMRTA1, P-Gene, AsA; DMRTA1, T-Gene, AsA; DMXL2, E-Gene, AsA; DNAH12, E-
Gene, AsA; DNAJA3, T-Gene, AsA; DNAJC1, P-Gene, AsA; DNER, P-Gene, AsA; DNM1P46, E-
Gene, AsA; DOC2A, E-Gene, AsA; DOK4, E-Gene, AsA; DOK6, E-Gene, AsA; DPAGT1, T-Gene,
AsA; DPCR1, T-Gene, AsA; DPF2, E-Gene, AsA; DPH6, E-Gene, AsA; DPH6-AS1, E-Gene, AsA;
DPP6, P-Gene, AsA; DPT, P-Gene, AsA; DPY19L2P3, E-Gene, AsA; DPY19L2P3, T-Gene, AsA;
DPY30, E-Gene, AsA; DPY30, P-Gene, AsA; DRAM1, P-Gene, AsA; DTNB, P-Gene, AsA;
DTWD2, P-Gene, AsA; DUSP11, T-Gene, AsA; DUSP18, E-Gene, AsA; DUSP18, P-Gene, AsA;
DUTP6, E-Gene, AsA; DYNLRB2, T-Gene, AsA; DYNLT1, E-Gene, AsA; E2F5, E-Gene, AsA;
EBLN2, E-Gene, AsA; ECHDC2, E-Gene, AsA; EDARADD, E-Gene, AsA; EDNRB, E-Gene, AsA;
EEFSEC, E-Gene, AsA; EEFSEC, P-Gene, AsA; EFEMP2, E-Gene, AsA; EGFR, P-Gene, AsA;
EHBP1L1, E-Gene, AsA; EHBP1L1, P-Gene, AsA; EIF1AD, E-Gene, AsA; EIF2AK1, E-Gene, AsA;
EIF2AK2, E-Gene, AsA; EIF2AK2, P-Gene, AsA; EIF2B3, E-Gene, AsA; EIF2C3, P-Gene, AsA;
EIF2S1, E-Gene, AsA; EIF3FP3, E-Gene, AsA; ELF1, P-Gene, AsA; ELMOD1, E-Gene, AsA;
ELMOD1, P-Gene, AsA; ELMOD2, E-Gene, AsA; ELN-AS1, E-Gene, AsA; ELOVL4, E-Gene, AsA;
ELP4, P-Gene, AsA; EMB, P-Gene, AsA; EMCN, E-Gene, AsA; EMCN, P-Gene, AsA; EML4, E-
Gene, AsA; ENDOU, E-Gene, AsA; ENOX1, P-Gene, AsA; ENPP7P12, E-Gene, AsA; ENTPD6, E-
Gene, AsA; EPB41L4A, E-Gene, AsA; EPHA3, E-Gene, AsA; EPHA3, P-Gene, AsA; EPHA4, P-
Gene, AsA; EPHB6, E-Gene, AsA; EPHB6, P-Gene, AsA; EPHX2, E-Gene, AsA; EPHX2, P-Gene,
AsA; EPM2A, P-Gene, AsA; ERBB2IP, P-Gene, AsA; ERBB4, E-Gene, AsA; ERBB4, P-Gene, AsA;
EREG, E-Gene, AsA; ERI2, E-Gene, AsA; ERN1, P-Gene, AsA; ERO1B, E-Gene, AsA; ESPNL, E-
Gene, AsA; ETFDH, E-Gene, AsA; ETS1, P-Gene, AsA; ETV2, E-Gene, AsA; EVA1B, E-Gene,
AsA; EXOSC4, E-Gene, AsA; EYS, P-Gene, AsA; F13A1, C-Gene, AsA; F13A1, P-Gene, AsA;
FAM116A, P-Gene, AsA; FAM133B, P-Gene, AsA; FAM13A, P-Gene, AsA; FAM149A, E-Gene,
AsA; FAM149A, P-Gene, AsA; FAM153C, E-Gene, AsA; FAM153C, P-Gene, AsA; FAM155A, P-
Gene, AsA; FAM163A, E-Gene, AsA; FAM167A, E-Gene, AsA; FAM167A-AS1, E-Gene, AsA;
FAM168A, T-Gene, AsA; FAM174A, P-Gene, AsA; FAM176A, P-Gene, AsA; FAM177A1, E-Gene,
AsA; FAM182A, E-Gene, AsA; FAM182B, E-Gene, AsA; FAM190A, P-Gene, AsA; FAM198B, E-
Gene, AsA; FAM198B, P-Gene, AsA; FAM198B-AS1, E-Gene, AsA; FAM20A, E-Gene, AsA;
FAM20A, P-Gene, AsA; FAM20B, E-Gene, AsA; FAM214A, E-Gene, AsA; FAM216A, E-Gene,
AsA; FAM220A, E-Gene, AsA; FAM47E, E-Gene, AsA; FAM57B, E-Gene, AsA; FAM65B, P-Gene,
AsA; FAM66A, E-Gene, AsA; FAM66D, E-Gene, AsA; FAM71F2, T-Gene, AsA; FAM81B, P-Gene,
AsA; FAM90A25P, E-Gene, AsA; FAN1, E-Gene, AsA; FANCC, P-Gene, AsA; FAR2P1, E-Gene,
AsA; FAR2P2, E-Gene, AsA; FASTKD2, E-Gene, AsA; FASTKD2, P-Gene, AsA; FAT3, P-Gene,
AsA; FBXL22, E-Gene, AsA; FBXO8, P-Gene, AsA; FBXW5, E-Gene, AsA; FCHSD2, P-Gene,
AsA; FCHSD2, T-Gene, AsA; FDFT1, E-Gene, AsA; FER, E-Gene, AsA; FER, P-Gene, AsA; FEZ2,
E-Gene, AsA; FGF19, P-Gene, AsA; FGF2, P-Gene, AsA; FGG, P-Gene, AsA; FIBP, E-Gene, AsA;
FIGN, E-Gene, AsA; FIGNL1, T-Gene, AsA; FKBP14, T-Gene, AsA; FKBP9, E-Gene, AsA; FKBPL,
P-Gene, AsA; FKBPL, T-Gene, AsA; FLJ20021, E-Gene, AsA; FLNB, E-Gene, AsA; FLNB-AS1, E-
Gene, AsA; FLNC, T-Gene, AsA; FLOT1, E-Gene, AsA; FLT1P1, E-Gene, AsA; FOLH1, P-Gene,
AsA; FOXD4L1, P-Gene, AsA; FOXL2, E-Gene, AsA; FOXP2, P-Gene, AsA; FOXR1, T-Gene,
AsA; FRAS1, P-Gene, AsA; FRMD4A, P-Gene, AsA; FRMD8, P-Gene, AsA; FRRS1, P-Gene, AsA;
FRS2, E-Gene, AsA; FRS2, P-Gene, AsA; FSTL5, P-Gene, AsA; FTH1P4, E-Gene, AsA; FTLP14, E-
Gene, AsA; FUK, P-Gene, AsA; FUT9, P-Gene, AsA; FYCO1, E-Gene, AsA; G2E3, P-Gene, AsA;
GABARAPL1, E-Gene, AsA; GABBR1, T-Gene, AsA; GABRG3, P-Gene, AsA; GAL3ST3, E-Gene,
AsA; GALM, P-Gene, AsA; GALNT13, P-Gene, AsA; GALNT15, E-Gene, AsA; GALNT17, E-
Gene, AsA; GALNT8, P-Gene, AsA; GALNTL2, P-Gene, AsA; GALNTL6, P-Gene, AsA; GALT, E-
Gene, AsA; GAN, T-Gene, AsA; GAP43, P-Gene, AsA; GAPDHP52, E-Gene, AsA; GAS1, E-Gene,
AsA; GAS5, E-Gene, AsA; GBE1, P-Gene, AsA; GBGT1, P-Gene, AsA; GCDH, E-Gene, AsA;
GCSAM, E-Gene, AsA; GCSH, E-Gene, AsA; GCSH, T-Gene, AsA; GDPD3, E-Gene, AsA; GFM1,
E-Gene, AsA; GFM1, P-Gene, AsA; GFPT2, C-Gene, AsA; GFPT2, E-Gene, AsA; GFPT2, P-Gene,
AsA; GGH, E-Gene, AsA; GLCE, P-Gene, AsA; GLRA1, E-Gene, AsA; GLS, E-Gene, AsA; GLS2,
E-Gene, AsA; GLTP, E-Gene, AsA; GMDS, P-Gene, AsA; GNB1, E-Gene, AsA; GNB5, E-Gene,
AsA; GNG5, E-Gene, AsA; GNG5, P-Gene, AsA; GNS, E-Gene, AsA; GOLGA2P5, E-Gene, AsA;
GOLGA8H, E-Gene, AsA; GOLGA8Q, E-Gene, AsA; GOLGA8R, E-Gene, AsA; GOLGA8T, E-
Gene, AsA; GOLGA8UP, E-Gene, AsA; GOLIM4, E-Gene, AsA; GOLPH3L, E-Gene, AsA; GP2, P-
Gene, AsA; GPATCH2, P-Gene, AsA; GPBP1, P-Gene, AsA; GPC6, P-Gene, AsA; GPN3, E-Gene,
AsA; GPN3, P-Gene, AsA; GPR110, P-Gene, AsA; GPR137B, P-Gene, AsA; GPR149, P-Gene, AsA;
GPR160, E-Gene, AsA; GPR160, P-Gene, AsA; GPR182, E-Gene, AsA; GPRIN2, P-Gene, AsA;
GPSM3, P-Gene, AsA; GPSM3, T-Gene, AsA; GPT, E-Gene, AsA; GPX5, P-Gene, AsA; GPX5, T-
Gene, AsA; GPX6, P-Gene, AsA; GPX6, T-Gene, AsA; GRHL3, P-Gene, AsA; GRIA4, E-Gene,
AsA; GRIA4, P-Gene, AsA; GRIP1, P-Gene, AsA; GS1-122H1.2, P-Gene, AsA; GS1-124K5.4, E-
Gene, AsA; GS1-211B7.1, P-Gene, AsA; GSC, P-Gene, AsA; GTF2E2, E-Gene, AsA; GTF2I, P-
Gene, AsA; GTF2IP23, E-Gene, AsA; GTF2IP5, E-Gene, AsA; GTF2IP7, E-Gene, AsA; GTF2IP9, E-
Gene, AsA; GTF2IRD1, P-Gene, AsA; GTF2IRD2, E-Gene, AsA; GULOP, E-Gene, AsA; GUSB, E-
Gene, AsA; GXYLT2, E-Gene, AsA; GZMA, E-Gene, AsA; H1FNT, E-Gene, AsA; H2AFX, T-Gene,
AsA; HACD2, E-Gene, AsA; HACE1, P-Gene, AsA; HAUS4, E-Gene, AsA; HBS1L, C-Gene, AsA;
HBS1L, E-Gene, AsA; HBS1L, P-Gene, AsA; HCG14, T-Gene, AsA; HCG15, T-Gene, AsA; HCG17,
P-Gene, AsA; HCG20, E-Gene, AsA; HCG21, T-Gene, AsA; HCG22, E-Gene, AsA; HCG23, P-Gene,
AsA; HCG23, T-Gene, AsA; HCG26, T-Gene, AsA; HCG9, T-Gene, AsA; HCN1, P-Gene, AsA;
HCP5, E-Gene, AsA; HEATR7B2, P-Gene, AsA; HECTD3, E-Gene, AsA; HERC1, E-Gene, AsA;
HERC2P10, E-Gene, AsA; HHLA3, E-Gene, AsA; HIBCH, E-Gene, AsA; HINFP, T-Gene, AsA;
HIP1, P-Gene, AsA; HIST1H1B, T-Gene, AsA; HIST1H2BE, P-Gene, AsA; HIST1H2BK, T-Gene,
AsA; HIST1H2BL, T-Gene, AsA; HIST1H4K, T-Gene, AsA; HIST1H4L, T-Gene, AsA; HIST3H2A,
E-Gene, AsA; HLA-DQB2, C-Gene, AsA; HMP19, P-Gene, AsA; HNRNPA1P4, E-Gene, AsA;
HNRNPA1P40, E-Gene, AsA; HOMEZ, E-Gene, AsA; HOMEZ, T-Gene, AsA; HORMAD1, E-Gene,
AsA; HOTAIR, E-Gene, AsA; HOTAIR, P-Gene, AsA; HSD17B3, P-Gene, AsA; HSDL2, E-Gene,
AsA; HSDL2, P-Gene, AsA; HSP90AB1, E-Gene, AsA; HSPA1A, T-Gene, AsA; HSPA1B, T-Gene,
AsA; HTR1E, P-Gene, AsA; HTR5A, P-Gene, AsA; HTRA2, T-Gene, AsA; HVCN1, E-Gene, AsA;
HYAL4, P-Gene, AsA; HYOU1, T-Gene, AsA; IAH1, E-Gene, AsA; IBA57, E-Gene, AsA; IFI27, P-
Gene, AsA; IFI27L1, E-Gene, AsA; IFI44, E-Gene, AsA; IFNA21, P-Gene, AsA; IFNGR1, T-Gene,
AsA; IFT81, E-Gene, AsA; IGHJ4, E-Gene, AsA; IKBKE, P-Gene, AsA; IKZF1, P-Gene, AsA;
IKZF1, T-Gene, AsA; IL11RA, E-Gene, AsA; IL15, E-Gene, AsA; IL15, P-Gene, AsA; IL17RD, E-
Gene, AsA; IL1RAP, P-Gene, AsA; IL20RB, E-Gene, AsA; IL20RB, P-Gene, AsA; IL25, T-Gene,
AsA; IL31RA, P-Gene, AsA; IL7R, P-Gene, AsA; ILDR2, E-Gene, AsA; IMP4, E-Gene, AsA;
IMPA2, E-Gene, AsA; IMPG2, E-Gene, AsA; INO80E, E-Gene, AsA; INPP4B, E-Gene, AsA;
INPP4B, P-Gene, AsA; INTS6-AS1, E-Gene, AsA; INTS8, E-Gene, AsA; INTU, P-Gene, AsA; IRF5,
E-Gene, AsA; IRF5, P-Gene, AsA; IRF5, T-Gene, AsA; ITGA3, C-Gene, AsA; ITGA3, P-Gene, AsA;
ITGAL, E-Gene, AsA; ITGB2, E-Gene, AsA; ITGB5, E-Gene, AsA; ITPR3, P-Gene, AsA; JAK2, P-
Gene, AsA; JAZF1, P-Gene, AsA; KALRN, P-Gene, AsA; KAT5, E-Gene, AsA; KATNBL1, E-Gene,
AsA; KCNG3, P-Gene, AsA; KCNH1, P-Gene, AsA; KCNH7, P-Gene, AsA; KCNH8, P-Gene, AsA;
KCNIP4, E-Gene, AsA; KCNIP4, P-Gene, AsA; KCNJ2, P-Gene, AsA; KCNK7, E-Gene, AsA;
KCNQ5, P-Gene, AsA; KCNT1, E-Gene, AsA; KCNT1, P-Gene, AsA; KCTD16, P-Gene, AsA;
KCTD7, E-Gene, AsA; KCTD7, P-Gene, AsA; KCTD8, P-Gene, AsA; KDM4A, P-Gene, AsA;
KERA, P-Gene, AsA; KIAA0319L, E-Gene, AsA; KIAA0391, E-Gene, AsA; KIAA1033, P-Gene,
AsA; KIAA1586, E-Gene, AsA; KIAA1958, E-Gene, AsA; KIAA1984, P-Gene, AsA; KIF24, E-
Gene, AsA; KIF26A, T-Gene, AsA; KIF2B, P-Gene, AsA; KIF5C, T-Gene, AsA; KIFC1, T-Gene,
AsA; KIFC2, E-Gene, AsA; KLB, E-Gene, AsA; KLC1, E-Gene, AsA; KLF12, E-Gene, AsA;
KLF12, P-Gene, AsA; KLF2P1, E-Gene, AsA; KLK14, E-Gene, AsA; KLK7, E-Gene, AsA; KLRC1,
E-Gene, AsA; KLRC2, E-Gene, AsA; KLRC3, E-Gene, AsA; KLRC4, E-Gene, AsA; KLRD1, E-
Gene, AsA; KLRD1, P-Gene, AsA; KLRK1, E-Gene, AsA; KRBA1, P-Gene, AsA; KREMEN1, E-
Gene, AsA; KRT16P2, E-Gene, AsA; KRT16P6, E-Gene, AsA; KRT17P1, E-Gene, AsA; KRT17P4,
E-Gene, AsA; KRT25, P-Gene, AsA; KRT8P26, E-Gene, AsA; KRTAP10-10, C-Gene, AsA;
KRTAP10-10, P-Gene, AsA; KRTAP13-1, P-Gene, AsA; KSR1, E-Gene, AsA; L3HYPDH, E-Gene,
AsA; LACC1, T-Gene, AsA; LACTB, P-Gene, AsA; LAMA2, P-Gene, AsA; LAMP1, P-Gene, AsA;
LARGE1, E-Gene, AsA; LARS2, E-Gene, AsA; LBH, E-Gene, AsA; LBH, P-Gene, AsA; LCN15, E-
Gene, AsA; LCN8, E-Gene, AsA; LGALS9, E-Gene, AsA; LHX4, E-Gene, AsA; LIAS, E-Gene,
AsA; LIAS, P-Gene, AsA; LINC00174, E-Gene, AsA; LINC00208, E-Gene, AsA; LINC00243, E-
Gene, AsA; LINC00271, E-Gene, AsA; LINC00271, T-Gene, AsA; LINC00284, T-Gene, AsA;
LINC00299, E-Gene, AsA; LINC00333, P-Gene, AsA; LINC00446, P-Gene, AsA; LINC00458, P-
Gene, AsA; LINC00471, P-Gene, AsA; LINC00478, P-Gene, AsA; LINC00501, P-Gene, AsA;
LINC00526, E-Gene, AsA; LINC00533, P-Gene, AsA; LINC00536, P-Gene, AsA; LINC00539, E-
Gene, AsA; LINC00636, E-Gene, AsA; LINC00638, E-Gene, AsA; LINC00667, E-Gene, AsA;
LINC00853, E-Gene, AsA; LINC00870, E-Gene, AsA; LINC00871, E-Gene, AsA; LINC01023, E-
Gene, AsA; LINC01047, E-Gene, AsA; LINC01170, E-Gene, AsA; LINC01192, E-Gene, AsA;
LINC01239, E-Gene, AsA; LINC01443, E-Gene, AsA; LINC01515, E-Gene, AsA; LINC01529, E-
Gene, AsA; LINC01592, E-Gene, AsA; LINC01697, E-Gene, AsA; LINC01783, E-Gene, AsA;
LINC01856, E-Gene, AsA; LINC01857, E-Gene, AsA; LINC02018, E-Gene, AsA; LINC02058, E-
Gene, AsA; LINC02147, E-Gene, AsA; LINC02152, E-Gene, AsA; LINC02170, E-Gene, AsA;
LINC02208, E-Gene, AsA; LINC02249, E-Gene, AsA; LINC02305, E-Gene, AsA; LINC02352, E-
Gene, AsA; LINC02398, E-Gene, AsA; LINC02432, E-Gene, AsA; LINC02462, E-Gene, AsA;
LINC02494, E-Gene, AsA; LINC02552, E-Gene, AsA; LINC02568, E-Gene, AsA; LL22NC03-
13G6.2, P-Gene, AsA; LLCFC1, E-Gene, AsA; LOC100130476, T-Gene, AsA; LOC100132735, T-
Gene, AsA; LPP, P-Gene, AsA; LRBA, E-Gene, AsA; LRBA, P-Gene, AsA; LRFN2, P-Gene, AsA;
LRFN5, E-Gene, AsA; LRFN5, P-Gene, AsA; LRP1, E-Gene, AsA; LRP2BP, E-Gene, AsA;
LRRC16A, P-Gene, AsA; LRRC18, E-Gene, AsA; LRRC28, E-Gene, AsA; LRRC34, E-Gene, AsA;
LRRC37B, E-Gene, AsA; LRRC39, E-Gene, AsA; LRRC52, E-Gene, AsA; LRRCC1, E-Gene, AsA;
LRRIQ1, P-Gene, AsA; LSAMP, P-Gene, AsA; LSM2, T-Gene, AsA; LSM8, E-Gene, AsA; LST1, P-
Gene, AsA; LST1, T-Gene, AsA; LTA, C-Gene, AsA; LTA, P-Gene, AsA; LTB, T-Gene, AsA;
LTBP1, P-Gene, AsA; LTBP3, E-Gene, AsA; LUZP2, P-Gene, AsA; LXN, E-Gene, AsA; LY6G6C,
E-Gene, AsA; LYPD6, T-Gene, AsA; LYPD6B, E-Gene, AsA; LYPD6B, P-Gene, AsA; LYPD6B, T-
Gene, AsA; LYSMD1, E-Gene, AsA; LYSMD2, E-Gene, AsA; LYSMD4, E-Gene, AsA; LYZL1, P-
Gene, AsA; MAB21L2, E-Gene, AsA; MAEL, E-Gene, AsA; MAGI2, P-Gene, AsA; MAMDC4, E-
Gene, AsA; MAN1A1, P-Gene, AsA; MAP2, E-Gene, AsA; MAP3K11, E-Gene, AsA; MAP3K20, E-
Gene, AsA; MAP7D1, E-Gene, AsA; MAPK13, E-Gene, AsA; MAPK14, E-Gene, AsA; MAPK14, P-
Gene, AsA; MAPK3, E-Gene, AsA; MAPK9, E-Gene, AsA; MAPRE2, E-Gene, AsA; MARCKS, P-
Gene, AsA; MARK3, E-Gene, AsA; MARS, E-Gene, AsA; MAS1L, P-Gene, AsA; MCCD1, P-Gene,
AsA; MCCD1, T-Gene, AsA; MCPH1, E-Gene, AsA; MCTP1, P-Gene, AsA; MDFIC, E-Gene, AsA;
MDFIC, P-Gene, AsA; MDH1B, E-Gene, AsA; MECOM, P-Gene, AsA; MED15P5, E-Gene, AsA;
MED15P9, E-Gene, AsA; MEF2A, P-Gene, AsA; Metazoa_SRP, P-Gene, AsA; METTL9, E-Gene,
AsA; MFF, E-Gene, AsA; MFSD1, E-Gene, AsA; MGAT3, E-Gene, AsA; MGAT4C, E-Gene, AsA;
MGAT4D, E-Gene, AsA; MGLL, E-Gene, AsA; MGST3, E-Gene, AsA; MGST3, P-Gene, AsA;
MICA, E-Gene, AsA; MICB, E-Gene, AsA; MICB, P-Gene, AsA; MICU2, E-Gene, AsA; MIOS, E-
Gene, AsA; MIPEPP3, E-Gene, AsA; MIPOL1, P-Gene, AsA; MIR1269A, P-Gene, AsA; MIR1289-2,
T-Gene, AsA; MIR219-1, T-Gene, AsA; MIR3135B, P-Gene, AsA; MIR3150BHG, E-Gene, AsA;
MIR3924, P-Gene, AsA; MIR4492, T-Gene, AsA; MIR4692, T-Gene, AsA; MIR5583-1, P-Gene,
AsA; MIR877, T-Gene, AsA; MLF1, E-Gene, AsA; MLIP, E-Gene, AsA; MLIP, P-Gene, AsA;
MLLT11, E-Gene, AsA; MLPH, E-Gene, AsA; MLPH, P-Gene, AsA; MMAA, E-Gene, AsA;
MMP23A, E-Gene, AsA; MOB1A, T-Gene, AsA; MOB1B, E-Gene, AsA; MOBKL1B, T-Gene, AsA;
MOCS1, P-Gene, AsA; MOG, P-Gene, AsA; MOGS, T-Gene, AsA; MPC2, E-Gene, AsA; MPIG6B,
E-Gene, AsA; MPP5, E-Gene, AsA; MPV17, P-Gene, AsA; MPZL1, E-Gene, AsA; MRM3, E-Gene,
AsA; MROH2B, E-Gene, AsA; MRPL13, P-Gene, AsA; MRPL19, P-Gene, AsA; MRPL48, E-Gene,
AsA; MRPL48, T-Gene, AsA; MRPL55, E-Gene, AsA; MRPS17P1, E-Gene, AsA; MRPS21, P-Gene,
AsA; MRPS6, P-Gene, AsA; MS4A10, C-Gene, AsA; MS4A10, P-Gene, AsA; MS4A6E, E-Gene,
AsA; MSRB2, T-Gene, AsA; MT1F, E-Gene, AsA; MTCO1P40, E-Gene, AsA; MTCO2P11, E-Gene,
AsA; MTDHP3, E-Gene, AsA; MTDHP4, E-Gene, AsA; MTHFD2, T-Gene, AsA; MTHFD2L, E-
Gene, AsA; MTHFD2L, P-Gene, AsA; MTMR10, E-Gene, AsA; MTMR10, P-Gene, AsA; MTSS1L,
E-Gene, AsA; MUC13, P-Gene, AsA; MUC22, E-Gene, AsA; MUC22, P-Gene, AsA; MUC22, T-
Gene, AsA; MUT, E-Gene, AsA; MYEOV, E-Gene, AsA; MYH6, T-Gene, AsA; MYH7, T-Gene,
AsA; MYL12A, E-Gene, AsA; MYL12B, E-Gene, AsA; MYL12B, P-Gene, AsA; MYNN, E-Gene,
AsA; MYNN, P-Gene, AsA; MYO1A, E-Gene, AsA; MYOM1, E-Gene, AsA; MYRF, E-Gene, AsA;
MZT2B, E-Gene, AsA; N6AMT1, E-Gene, AsA; NAA10, P-Gene, AsA; NAAA, E-Gene, AsA;
NAALADL2, P-Gene, AsA; NAB2, E-Gene, AsA; NAF1, E-Gene, AsA; NANP, E-Gene, AsA;
NAT1, P-Gene, AsA; NAV2, P-Gene, AsA; NBN, E-Gene, AsA; NBPF1, E-Gene, AsA; NCAM2, P-
Gene, AsA; NCR3, E-Gene, AsA; NCR3, P-Gene, AsA; NCR3, T-Gene, AsA; NDEL1, E-Gene, AsA;
NDEL1, P-Gene, AsA; NDST4, P-Gene, AsA; NDUFAF6, E-Gene, AsA; NEAT1, E-Gene, AsA;
NEDD1, E-Gene, AsA; NEDD1, T-Gene, AsA; NEFH, E-Gene, AsA; NEGR1, P-Gene, AsA; NEIL2,
E-Gene, AsA; NEK1, P-Gene, AsA; NEK2, E-Gene, AsA; NELL1, P-Gene, AsA; NEU1, T-Gene,
AsA; NEU3, E-Gene, AsA; NEUROD6, P-Gene, AsA; NFATC3, E-Gene, AsA; NFKBIL1, P-Gene,
AsA; NFKBIL1, T-Gene, AsA; NID1, P-Gene, AsA; NINL, E-Gene, AsA; NIPAL3, E-Gene, AsA;
NKAIN2, P-Gene, AsA; NKAIN3, P-Gene, AsA; NKAPL, T-Gene, AsA; NLGN1, P-Gene, AsA;
NLRC4, E-Gene, AsA; NLRX1, T-Gene, AsA; NMNAT3, E-Gene, AsA; NMUR2, E-Gene, AsA;
NOC2LP1, E-Gene, AsA; NOL4, P-Gene, AsA; NOS2, P-Gene, AsA; NOS2P4, E-Gene, AsA;
NOTCH3, P-Gene, AsA; NOTCH4, E-Gene, AsA; NOTCH4, P-Gene, AsA; NOTCH4, T-Gene, AsA;
NOVA1-AS1, P-Gene, AsA; NOX5, E-Gene, AsA; NPAP1P6, E-Gene, AsA; NPAS3, P-Gene, AsA;
NPIPB11, E-Gene, AsA; NPIPB12, E-Gene, AsA; NPIPB14P, E-Gene, AsA; NPY1R, E-Gene, AsA;
NPY1R, P-Gene, AsA; NPY2R, E-Gene, AsA; NPY5R, E-Gene, AsA; NPY5R, P-Gene, AsA;
NR2F2, P-Gene, AsA; NRBF2P2, E-Gene, AsA; NRXN3, P-Gene, AsA; NT5C3, P-Gene, AsA;
NT5C3A, E-Gene, AsA; NTF3, P-Gene, AsA; NTPCR, E-Gene, AsA; NTS, E-Gene, AsA; NUBPL,
P-Gene, AsA; NUDT19P5, E-Gene, AsA; NUDT2, E-Gene, AsA; NUP210L, E-Gene, AsA;
NUP210L, P-Gene, AsA; NXPE3, E-Gene, AsA; NYAP2, P-Gene, AsA; OAS2, E-Gene, AsA; OAS2,
P-Gene, AsA; OAS3, E-Gene, AsA; OBP2B, E-Gene, AsA; OBSCN, E-Gene, AsA; OBSCN-AS1, E-
Gene, AsA; OCM, E-Gene, AsA; ODF1, P-Gene, AsA; ODZ2, P-Gene, AsA; OLIG3, T-Gene, AsA;
OPN1SW, T-Gene, AsA; OPTC, P-Gene, AsA; OR10A7, P-Gene, AsA; OR11A1, P-Gene, AsA;
OR2B6, P-Gene, AsA; OR2H1, P-Gene, AsA; OR2J3, P-Gene, AsA; OR2L13, E-Gene, AsA;
OR2L13, P-Gene, AsA; OR2L1P, E-Gene, AsA; OR2M1P, E-Gene, AsA; OR2M4, P-Gene, AsA;
OR2M5, P-Gene, AsA; OR4K1, P-Gene, AsA; OR52A5, P-Gene, AsA; OR5AC2, T-Gene, AsA;
OR5AC4P, E-Gene, AsA; OR5H15, P-Gene, AsA; OR5H2, T-Gene, AsA; OR5H5P, E-Gene, AsA;
OR5K4, T-Gene, AsA; OR5V1, P-Gene, AsA; ORC3, P-Gene, AsA; ORMDL1, E-Gene, AsA;
OSGEPL1, E-Gene, AsA; OSGEPL1-AS1, E-Gene, AsA; OSGIN2, E-Gene, AsA; OVOL1, E-Gene,
AsA; OVOL1, P-Gene, AsA; OXER1, E-Gene, AsA; OXGR1, P-Gene, AsA; P2RX5, E-Gene, AsA;
P2RY2, T-Gene, AsA; P2RY6, T-Gene, AsA; P4HA1, P-Gene, AsA; PAAF1, E-Gene, AsA;
PABPC4L, E-Gene, AsA; PABPN1, T-Gene, AsA; PADI4, E-Gene, AsA; PADI4, P-Gene, AsA;
PAK7, P-Gene, AsA; PANK4, P-Gene, AsA; PAPOLB, E-Gene, AsA; PAQR5, E-Gene, AsA;
PARM1, E-Gene, AsA; PARP11, E-Gene, AsA; PARP11, P-Gene, AsA; PARP8, E-Gene, AsA;
PARPBP, E-Gene, AsA; PCAT18, E-Gene, AsA; PCDH10, E-Gene, AsA; PCDH15, P-Gene, AsA;
PCDHA1, P-Gene, AsA; PCNX3, E-Gene, AsA; PCNXL2, P-Gene, AsA; PCNXL3, P-Gene, AsA;
PCSK2, P-Gene, AsA; PDCD10, E-Gene, AsA; PDCL3P4, E-Gene, AsA; PDE2A, E-Gene, AsA;
PDE2A, T-Gene, AsA; PDE4D, P-Gene, AsA; PDE7B, T-Gene, AsA; PDE8B, E-Gene, AsA; PDF, E-
Gene, AsA; PDHX, E-Gene, AsA; PDHX, P-Gene, AsA; PDIA6, E-Gene, AsA; PDIA6, P-Gene,
AsA; PDXK, E-Gene, AsA; PDZRN3, E-Gene, AsA; PDZRN4, P-Gene, AsA; PELP1, E-Gene, AsA;
PERP, T-Gene, AsA; PEX13, E-Gene, AsA; PFKM, E-Gene, AsA; PGA3, E-Gene, AsA; PGAM1P8,
E-Gene, AsA; PGBD1, T-Gene, AsA; PGR, P-Gene, AsA; PHACTR1, P-Gene, AsA; PHC3, E-Gene,
AsA; PHF1, T-Gene, AsA; PHF15, T-Gene, AsA; PHLDB1, T-Gene, AsA; PHPT1, E-Gene, AsA;
PI4K2B, E-Gene, AsA; PID1, P-Gene, AsA; PIK3C3, E-Gene, AsA; PIK3C3, P-Gene, AsA;
PIKFYVE, E-Gene, AsA; PIP4K2A, E-Gene, AsA; PIP4K2A, T-Gene, AsA; PKD1L2, T-Gene, AsA;
PKP4, E-Gene, AsA; PLA2G15, E-Gene, AsA; PLA2G16, E-Gene, AsA; PLA2G7, E-Gene, AsA;
PLAC9P1, E-Gene, AsA; PLCB1, P-Gene, AsA; PLCB4, E-Gene, AsA; PLCD4, E-Gene, AsA;
PLD4, E-Gene, AsA; PLEK2, E-Gene, AsA; PLEK2, P-Gene, AsA; PLEKHB1, E-Gene, AsA; PLK3,
P-Gene, AsA; PLXDC1, P-Gene, AsA; PMCH, P-Gene, AsA; PMS1, E-Gene, AsA; PMS1, P-Gene,
AsA; PMS2, E-Gene, AsA; PMS2, P-Gene, AsA; PMS2CL, E-Gene, AsA; PMS2P3, E-Gene, AsA;
PMS2P5, E-Gene, AsA; PODN, T-Gene, AsA; POGK, E-Gene, AsA; POGK, P-Gene, AsA; POGZ,
E-Gene, AsA; POLA2, E-Gene, AsA; POLR2B, P-Gene, AsA; POLR2C, E-Gene, AsA; POM121L2,
P-Gene, AsA; POTED, P-Gene, AsA; POU5F1, E-Gene, AsA; POU5F1, T-Gene, AsA; PPARGC1B,
E-Gene, AsA; PPARGC1B, P-Gene, AsA; PPEF2, P-Gene, AsA; PPHLN1, E-Gene, AsA; PPIAP84,
E-Gene, AsA; PPIB, E-Gene, AsA; PPID, E-Gene, AsA; PPP1CC, E-Gene, AsA; PPP1R11, T-Gene,
AsA; PPP1R13B, E-Gene, AsA; PPP1R18, E-Gene, AsA; PPP1R1A, C-Gene, AsA; PPP1R1A, P-
Gene, AsA; PPP1R8, P-Gene, AsA; PPP1R9A, P-Gene, AsA; PPP2CA, T-Gene, AsA; PPP2R3C, E-
Gene, AsA; PPP4R2, E-Gene, AsA; PPP4R2, P-Gene, AsA; PPTC7, E-Gene, AsA; PQLC3, E-Gene,
AsA; PRDX1, E-Gene, AsA; PRDX6, E-Gene, AsA; PRELID2, P-Gene, AsA; PRKACB, P-Gene,
AsA; PRKCB, P-Gene, AsA; PRKCI, P-Gene, AsA; PRKG1, P-Gene, AsA; PRMT5, E-Gene, AsA;
PRMT5, T-Gene, AsA; PRPF4B, P-Gene, AsA; PRR15, E-Gene, AsA; PRR15, T-Gene, AsA;
PRRC2A, P-Gene, AsA; PRRC2A, T-Gene, AsA; PRRT1, E-Gene, AsA; PRRT1, P-Gene, AsA;
PRRT1, T-Gene, AsA; PRSS16, T-Gene, AsA; PRSS42, E-Gene, AsA; PRSS48, E-Gene, AsA;
PRUNE, P-Gene, AsA; PRUNE1, E-Gene, AsA; PSD3, P-Gene, AsA; PSMA6, E-Gene, AsA;
PSMB11, E-Gene, AsA; PSMB5, E-Gene, AsA; PSMB5, P-Gene, AsA; PSMB8, T-Gene, AsA;
PSMB9, P-Gene, AsA; PSMB9, T-Gene, AsA; PSORS1C1, E-Gene, AsA; PSORS1C1, T-Gene, AsA;
PSORS1C2, E-Gene, AsA; PSORS1C2, T-Gene, AsA; PSORS1C3, E-Gene, AsA; PTAFR, E-Gene,
AsA; PTENP1, E-Gene, AsA; PTGDR, E-Gene, AsA; PTGDR, P-Gene, AsA; PTGS2, P-Gene, AsA;
PTH2R, P-Gene, AsA; PTPN18, E-Gene, AsA; PTPN18, T-Gene, AsA; PTPRC, P-Gene, AsA;
PTPRD, P-Gene, AsA; PTPRK, P-Gene, AsA; PYGB, E-Gene, AsA; RAB17, E-Gene, AsA; RAB23,
E-Gene, AsA; RAB3C, P-Gene, AsA; RAB44, E-Gene, AsA; RAB6A, E-Gene, AsA; RAB6C, E-
Gene, AsA; RAB6C, P-Gene, AsA; RAB6C-AS1, E-Gene, AsA; RABGAP1L, E-Gene, AsA;
RABGEF1, E-Gene, AsA; RABGEF1, P-Gene, AsA; RAD23B, P-Gene, AsA; RAD51B, P-Gene,
AsA; RAD9B, E-Gene, AsA; RADIL, E-Gene, AsA; RADIL, P-Gene, AsA; RALGPS2, P-Gene,
AsA; RALYL, P-Gene, AsA; RAPGEF2, P-Gene, AsA; RAPGEF3, E-Gene, AsA; RAPGEF4, E-
Gene, AsA; RARRES1, E-Gene, AsA; RASA1, E-Gene, AsA; RASGRP1, E-Gene, AsA; RASGRP3,
P-Gene, AsA; RASSF9, E-Gene, AsA; RBFOX3, P-Gene, AsA; RBM23, E-Gene, AsA; RBM42, E-
Gene, AsA; RBM45, P-Gene, AsA; RCAN2, E-Gene, AsA; RCAN3, E-Gene, AsA; RD3L, E-Gene,
AsA; RDH16, E-Gene, AsA; RDH16, P-Gene, AsA; RELT, T-Gene, AsA; REPS1, T-Gene, AsA;
REXO5, E-Gene, AsA; RFLNB, E-Gene, AsA; RFPL2, P-Gene, AsA; RGS1, P-Gene, AsA; RGS21,
P-Gene, AsA; RHOJ, E-Gene, AsA; RHOJ, P-Gene, AsA; RHOT1, E-Gene, AsA; RIC3, E-Gene,
AsA; RIPK2, E-Gene, AsA; RIPK2, T-Gene, AsA; RIPPLY2, P-Gene, AsA; RMST, P-Gene, AsA;
RN5S117, P-Gene, AsA; RN5S144, P-Gene, AsA; RN5S182, P-Gene, AsA; RN5S38, P-Gene, AsA;
RN5S384, P-Gene, AsA; RN5S64, P-Gene, AsA; RN5S79, P-Gene, AsA; RN7SL138P, E-Gene, AsA;
RN7SL239P, E-Gene, AsA; RNASE10, P-Gene, AsA; RNASEH2C, E-Gene, AsA; RNF11, P-Gene,
AsA; RNF114, P-Gene, AsA; RNF149, P-Gene, AsA; RNF169, E-Gene, AsA; RNF169, P-Gene,
AsA; RNF187, E-Gene, AsA; RNF212B, E-Gene, AsA; RNF216P1, E-Gene, AsA; RNF25, E-Gene,
AsA; RNF5, E-Gene, AsA; RNU6-36, P-Gene, AsA; RNU6-38, P-Gene, AsA; RNU6-54, P-Gene,
AsA; RNU6-62, P-Gene, AsA; RNU7-14P, P-Gene, AsA; RNU7-16P, T-Gene, AsA; RNU7-63P, T-
Gene, AsA; RNY4P27, P-Gene, AsA; ROBO2, P-Gene, AsA; ROPN1, E-Gene, AsA; RP1-153P14.8,
P-Gene, AsA; RP1-23E21.2, P-Gene, AsA; RP1-269M15.3, P-Gene, AsA; RP1-35C21.2, P-Gene,
AsA; RP1-90J4.1, P-Gene, AsA; RP11-1080G15.1, P-Gene, AsA; RP11-1084I9.3, P-Gene, AsA;
RP11-10J5.1, P-Gene, AsA; RP11-10O22.1, P-Gene, AsA; RP11-10O22.2, P-Gene, AsA; RP11-
110I1.11, T-Gene, AsA; RP11-110I1.12, T-Gene, AsA; RP11-1114I9.1, P-Gene, AsA; RP11-
111A21.1, P-Gene, AsA; RP11-114H21.2, P-Gene, AsA; RP11-114J13.1, P-Gene, AsA; RP11-
115F18.1, P-Gene, AsA; RP11-123M21.1, P-Gene, AsA; RP11-124D2.2, T-Gene, AsA; RP11-
124D2.3, T-Gene, AsA; RP11-128D14.1, P-Gene, AsA; RP11-129M6.1, P-Gene, AsA; RP11-
130F10.1, P-Gene, AsA; RP11-131K5.1, P-Gene, AsA; RP11-139J15.5, P-Gene, AsA; RP11-
147L13.2, P-Gene, AsA; RP11-151E14.1, P-Gene, AsA; RP11-152K4.2, P-Gene, AsA; RP11-
152L20.3, P-Gene, AsA; RP11-158L12.5, P-Gene, AsA; RP11-169D4.2, T-Gene, AsA; RP11-
173D9.3, P-Gene, AsA; RP11-180M15.3, P-Gene, AsA; RP11-18F14.1, P-Gene, AsA; RP11-1A16.1,
P-Gene, AsA; RP11-202H2.1, P-Gene, AsA; RP11-204C23.1, P-Gene, AsA; RP11-20B7.1, P-Gene,
AsA; RP11-215P8.3, T-Gene, AsA; RP11-21C17.1, P-Gene, AsA; RP11-231E6.1, P-Gene, AsA;
RP11-240A16.1, P-Gene, AsA; RP11-246K15.1, P-Gene, AsA; RP11-251P6.1, P-Gene, AsA; RP11-
260M19.1, P-Gene, AsA; RP11-260O18.1, P-Gene, AsA; RP11-264E20.1, P-Gene, AsA; RP11-
272B17.1, P-Gene, AsA; RP11-275I4.2, P-Gene, AsA; RP11-277P12.20, P-Gene, AsA; RP11-
279O17.2, P-Gene, AsA; RP11-286H14.8, T-Gene, AsA; RP11-292B1.2, P-Gene, AsA; RP11-
296O14.3, P-Gene, AsA; RP11-298I3.5, T-Gene, AsA; RP11-298O21.5, P-Gene, AsA; RP11-
299H22.7, P-Gene, AsA; RP11-2G1.1, P-Gene, AsA; RP11-305B6.3, P-Gene, AsA; RP11-309L24.2,
T-Gene, AsA; RP11-30J20.1, P-Gene, AsA; RP11-313L9.1, P-Gene, AsA; RP11-314D7.4, P-Gene,
AsA; RP11-314O13.1, P-Gene, AsA; RP11-315H15.3, P-Gene, AsA; RP11-315I14.3, T-Gene, AsA;
RP11-318I4.1, P-Gene, AsA; RP11-319E16.1, P-Gene, AsA; RP11-321L2.2, P-Gene, AsA; RP11-
326L24.4, P-Gene, AsA; RP11-328K4.1, P-Gene, AsA; RP11-344L13.2, P-Gene, AsA; RP11-
348J24.2, P-Gene, AsA; RP11-349J5.2, T-Gene, AsA; RP11-353P15.1, P-Gene, AsA; RP11-356I2.2,
P-Gene, AsA; RP11-356I2.4, P-Gene, AsA; RP11-35O7.1, P-Gene, AsA; RP11-360A9.2, P-Gene,
AsA; RP11-367G18.1, P-Gene, AsA; RP11-379J13.2, P-Gene, AsA; RP11-37B2.1, P-Gene, AsA;
RP11-384J4.2, P-Gene, AsA; RP11-390P2.4, T-Gene, AsA; RP11-394G3.2, P-Gene, AsA; RP11-
395D3.1, P-Gene, AsA; RP11-398A8.3, P-Gene, AsA; RP11-399D6.2, P-Gene, AsA; RP11-
403N16.2, P-Gene, AsA; RP11-413B19.2, P-Gene, AsA; RP11-415C15.2, P-Gene, AsA; RP11-
420N3.2, P-Gene, AsA; RP11-423J7.1, P-Gene, AsA; RP11-428G2.1, P-Gene, AsA; RP11-429O1.1,
P-Gene, AsA; RP11-436F21.1, P-Gene, AsA; RP11-442N1.1, P-Gene, AsA; RP11-445F6.2, T-Gene,
AsA; RP11-451L19.1, P-Gene, AsA; RP11-452B18.2, P-Gene, AsA; RP11-456I15.2, T-Gene, AsA;
RP11-460H9.1, P-Gene, AsA; RP11-463J7.2, P-Gene, AsA; RP11-469N6.3, P-Gene, AsA; RP11-
489P6.1, P-Gene, AsA; RP11-497D6.4, P-Gene, AsA; RP11-521D12.5, P-Gene, AsA; RP11-
523O18.7, P-Gene, AsA; RP11-525K10.3, P-Gene, AsA; RP11-526F3.1, P-Gene, AsA; RP11-
527H14.2, P-Gene, AsA; RP11-531P20.1, P-Gene, AsA; RP11-534N16.1, P-Gene, AsA; RP11-
542B15.1, P-Gene, AsA; RP11-542P2.1, P-Gene, AsA; RP11-545P6.2, P-Gene, AsA; RP11-
554A11.8, P-Gene, AsA; RP11-572M18.1, P-Gene, AsA; RP11-577G20.2, P-Gene, AsA; RP11-
594C13.2, P-Gene, AsA; RP11-611L7.1, P-Gene, AsA; RP11-617I14.1, P-Gene, AsA; RP11-
618G20.1, P-Gene, AsA; RP11-622K12.1, P-Gene, AsA; RP11-631B21.2, P-Gene, AsA; RP11-
63B19.1, P-Gene, AsA; RP11-649A16.1, P-Gene, AsA; RP11-64D24.2, P-Gene, AsA; RP11-
653B10.1, P-Gene, AsA; RP11-655G22.1, P-Gene, AsA; RP11-655H13.2, P-Gene, AsA; RP11-
655M19.1, P-Gene, AsA; RP11-65D17.1, P-Gene, AsA; RP11-65D24.2, P-Gene, AsA; RP11-
660M18.2, P-Gene, AsA; RP11-662J14.1, P-Gene, AsA; RP11-672A2.4, P-Gene, AsA; RP11-
676F20.1, P-Gene, AsA; RP11-679C8.2, P-Gene, AsA; RP11-686G23.2, P-Gene, AsA; RP11-6C14.1,
P-Gene, AsA; RP11-702M1.1, P-Gene, AsA; RP11-707G14.1, T-Gene, AsA; RP11-719J20.1, P-Gene,
AsA; RP11-72L22.1, P-Gene, AsA; RP11-731J8.2, P-Gene, AsA; RP11-731N10.1, P-Gene, AsA;
RP11-756H20.1, P-Gene, AsA; RP11-764E7.1, P-Gene, AsA; RP11-770J1.4, T-Gene, AsA; RP11-
788H18.1, P-Gene, AsA; RP11-799O21.1, P-Gene, AsA; RP11-800A3.2, T-Gene, AsA; RP11-
802H3.2, P-Gene, AsA; RP11-804N13.1, P-Gene, AsA; RP11-809F4.3, P-Gene, AsA; RP11-
809H16.2, P-Gene, AsA; RP11-835E18.5, P-Gene, AsA; RP11-849H4.2, T-Gene, AsA; RP11-
886D15.1, P-Gene, AsA; RP11-8L2.1, P-Gene, AsA; RP11-91K9.1, P-Gene, AsA; RP11-945A11.1,
P-Gene, AsA; RP11-94P11.4, P-Gene, AsA; RP11-96H19.1, P-Gene, AsA; RP11-996F15.2, P-Gene,
AsA; RP11-99H20.1, P-Gene, AsA; RP13-143G15.3, T-Gene, AsA; RP13-143G15.4, T-Gene, AsA;
RP3-410C9.1, P-Gene, AsA; RP3-449H6.1, P-Gene, AsA; RP3-463P15.1, P-Gene, AsA; RP3-
523C21.1, P-Gene, AsA; RP4-651E10.4, P-Gene, AsA; RP4-735C1.6, P-Gene, AsA; RP4-745K6.1, P-
Gene, AsA; RP4-764O22.2, P-Gene, AsA; RP5-1010E17.1, P-Gene, AsA; RP5-1029F21.4, P-Gene,
AsA; RP5-1186N24.3, P-Gene, AsA; RP5-1186P10.1, P-Gene, AsA; RP5-837I24.2, P-Gene, AsA;
RP5-839B4.8, P-Gene, AsA; RP5-858B6.1, P-Gene, AsA; RP9P, E-Gene, AsA; RPAP3, E-Gene,
AsA; RPF1, E-Gene, AsA; RPL10A, E-Gene, AsA; RPL13AP2, E-Gene, AsA; RPL23AP64, T-Gene,
AsA; RPL3, E-Gene, AsA; RPL6, E-Gene, AsA; RPL7AP10, E-Gene, AsA; RPL8, E-Gene, AsA;
RPL9, E-Gene, AsA; RPLP0P2, E-Gene, AsA; RPP21, P-Gene, AsA; RPP21, T-Gene, AsA; RPS10,
E-Gene, AsA; RPS18, T-Gene, AsA; RPS25, T-Gene, AsA; RPS27L, E-Gene, AsA; RPS4XP16, E-
Gene, AsA; RPS5, E-Gene, AsA; RPS6, P-Gene, AsA; RPS6KA5, E-Gene, AsA; RPS6KA5, P-Gene,
AsA; RPS8, E-Gene, AsA; RPUSD3, E-Gene, AsA; RRP15, E-Gene, AsA; RRP15, P-Gene, AsA;
RSAD2, P-Gene, AsA; RSPH10B, E-Gene, AsA; RSPH10B2, E-Gene, AsA; RSRC1, E-Gene, AsA;
RTCA, E-Gene, AsA; RTKN, T-Gene, AsA; RTKN2, E-Gene, AsA; RTKN2, P-Gene, AsA; RUFY3,
E-Gene, AsA; RUVBL1, E-Gene, AsA; RXFP1, E-Gene, AsA; S100PBP, P-Gene, AsA; SAMD5, P-
Gene, AsA; SAPCD1, P-Gene, AsA; SAR1A, P-Gene, AsA; SASS6, E-Gene, AsA; SATB1, P-Gene,
AsA; SBSN, E-Gene, AsA; SCAF4, P-Gene, AsA; SCAND3, P-Gene, AsA; SCAND3, T-Gene, AsA;
SCN2A, E-Gene, AsA; SCN2A, P-Gene, AsA; SCOC, E-Gene, AsA; SCP2, E-Gene, AsA; SCUBE3,
E-Gene, AsA; SDK1, P-Gene, AsA; SDR9C7, E-Gene, AsA; SEC61A1, E-Gene, AsA; SELENBP1,
E-Gene, AsA; SENP1, P-Gene, AsA; SENP7, E-Gene, AsA; SEPHS1P1, E-Gene, AsA; SEPHS2, E-
Gene, AsA; SEPSECS, P-Gene, AsA; SERP2, T-Gene, AsA; SERPINC1, E-Gene, AsA; SERPINE3,
P-Gene, AsA; SERPINI2, P-Gene, AsA; SF3A3, P-Gene, AsA; SFMBT1, P-Gene, AsA; SFTA2, E-
Gene, AsA; SGCZ, P-Gene, AsA; SH2D4A, E-Gene, AsA; SHANK2, P-Gene, AsA; SIGLEC12, E-
Gene, AsA; SIGLEC6, C-Gene, AsA; SIGLEC6, P-Gene, AsA; SIK2, P-Gene, AsA; SIPA1, E-Gene,
AsA; SIPA1L2, P-Gene, AsA; SKIL, E-Gene, AsA; SKIV2L, E-Gene, AsA; SKIV2L, P-Gene, AsA;
SKP1, E-Gene, AsA; SKP1, T-Gene, AsA; SKP2, E-Gene, AsA; SLC12A2, P-Gene, AsA; SLC14A1,
P-Gene, AsA; SLC15A4, P-Gene, AsA; SLC16A7, E-Gene, AsA; SLC17A2, P-Gene, AsA; SLC1A7,
T-Gene, AsA; SLC22A10, E-Gene, AsA; SLC22A10, P-Gene, AsA; SLC22A17, T-Gene, AsA;
SLC22A20P, E-Gene, AsA; SLC22A25, E-Gene, AsA; SLC25A27, E-Gene, AsA; SLC26A8, E-
Gene, AsA; SLC29A1, P-Gene, AsA; SLC30A1, P-Gene, AsA; SLC30A6, E-Gene, AsA; SLC30A8,
P-Gene, AsA; SLC30A9, E-Gene, AsA; SLC30A9, P-Gene, AsA; SLC35A1, E-Gene, AsA;
SLC35A3, E-Gene, AsA; SLC35E4, E-Gene, AsA; SLC35F1, P-Gene, AsA; SLC36A4, P-Gene, AsA;
SLC37A4, T-Gene, AsA; SLC38A11, E-Gene, AsA; SLC39A10, P-Gene, AsA; SLC39A11, P-Gene,
AsA; SLC39A7, T-Gene, AsA; SLC44A1, P-Gene, AsA; SLC44A4, C-Gene, AsA; SLC44A4, P-
Gene, AsA; SLC48A1, E-Gene, AsA; SLC4A1AP, P-Gene, AsA; SLC4A5, T-Gene, AsA; SLC7A6,
E-Gene, AsA; SLC7A7, E-Gene, AsA; SLC7A8, T-Gene, AsA; SLC9A5, P-Gene, AsA; SLC9A9, P-
Gene, AsA; SLC9A9-AS1, E-Gene, AsA; SLC9C1, C-Gene, AsA; SLC9C1, E-Gene, AsA; SLC9C1,
P-Gene, AsA; SLCO2B1, E-Gene, AsA; SLIT2, P-Gene, AsA; SLITRK5, P-Gene, AsA; SLMAP, E-
Gene, AsA; SLMAP, P-Gene, AsA; SMAD1, P-Gene, AsA; SMCHD1, E-Gene, AsA; SMG1P5, E-
Gene, AsA; SMG1P6, E-Gene, AsA; SMIM18, E-Gene, AsA; SMIM2, E-Gene, AsA; SMIM2-AS1,
E-Gene, AsA; SMIM2-IT1, T-Gene, AsA; SMPD4, E-Gene, AsA; SMPDL3B, E-Gene, AsA;
SMYD3, E-Gene, AsA; snoMe28S-Am2634.2, T-Gene, AsA; SNORA38, T-Gene, AsA;
SNORA40.16, T-Gene, AsA; SNORA70, P-Gene, AsA; SNORD117, T-Gene, AsA; SNORD59, P-
Gene, AsA; SNORD78, P-Gene, AsA; SNORD84, T-Gene, AsA; snoU13, P-Gene, AsA; snoU13.218,
T-Gene, AsA; snoU13.248, T-Gene, AsA; snoU13.304, T-Gene, AsA; snoU13.57, T-Gene, AsA;
SNRNP27, P-Gene, AsA; SNX22, E-Gene, AsA; SNX25, E-Gene, AsA; SNX27, P-Gene, AsA;
SNX32, E-Gene, AsA; SOAT1, E-Gene, AsA; SOD1, E-Gene, AsA; SORBS2, P-Gene, AsA;
SORCS3, P-Gene, AsA; SOS2, P-Gene, AsA; SOSTDC1, P-Gene, AsA; SOX1, P-Gene, AsA; SOX5,
P-Gene, AsA; SOX9-AS1, E-Gene, AsA; SPACA1, P-Gene, AsA; SPAST, E-Gene, AsA; SPATA1,
E-Gene, AsA; SPATA17, P-Gene, AsA; SPCS2, E-Gene, AsA; SPCS3, P-Gene, AsA; SPDYE12P, E-
Gene, AsA; SPDYE5, E-Gene, AsA; SPESP1, T-Gene, AsA; SPINK5, E-Gene, AsA; SPINK5, P-
Gene, AsA; SPINK6, E-Gene, AsA; SPRYD4, E-Gene, AsA; SRBD1, P-Gene, AsA; SRP54, E-Gene,
AsA; SRP54-AS1, E-Gene, AsA; SRPK1, E-Gene, AsA; SRRM1, P-Gene, AsA; SRRM3, P-Gene,
AsA; SSR1, E-Gene, AsA; SSTR2, E-Gene, AsA; ST3GAL5, P-Gene, AsA; ST6GAL1, E-Gene,
AsA; ST6GAL1, P-Gene, AsA; ST8SIA4, P-Gene, AsA; STAG3L1, E-Gene, AsA; STAG3L4, E-
Gene, AsA; STAM, E-Gene, AsA; STAM, P-Gene, AsA; STAM-AS1, E-Gene, AsA; STAMBP, T-
Gene, AsA; STARD10, E-Gene, AsA; STARD10, T-Gene, AsA; STARD10-AS1, T-Gene, AsA;
STAT1, P-Gene, AsA; STAT4, E-Gene, AsA; STAT4, P-Gene, AsA; STAT6, E-Gene, AsA;
STEAP1B, E-Gene, AsA; STEAP1B, P-Gene, AsA; STK19, P-Gene, AsA; STK19, T-Gene, AsA;
STK19B, E-Gene, AsA; STK36, E-Gene, AsA; STK36, P-Gene, AsA; STK40, E-Gene, AsA; STPG1,
E-Gene, AsA; STX7, P-Gene, AsA; STYXL1, E-Gene, AsA; SULF1, E-Gene, AsA; SURF6, E-Gene,
AsA; SUSD1, E-Gene, AsA; SUZ12, E-Gene, AsA; SV2C, P-Gene, AsA; SYBU, P-Gene, AsA;
SYNGR1, E-Gene, AsA; SYNGR1, P-Gene, AsA; SYNGR2, E-Gene, AsA; SYNM, E-Gene, AsA;
SYT17, P-Gene, AsA; SYT9, P-Gene, AsA; SYTL3, E-Gene, AsA; TAB1, E-Gene, AsA; TAC3, E-
Gene, AsA; TADA1, E-Gene, AsA; TADA3, E-Gene, AsA; TAGLN3, E-Gene, AsA; TAP1, T-Gene,
AsA; TAP2, T-Gene, AsA; TAPBP, T-Gene, AsA; TBC1D23, E-Gene, AsA; TBC1D30, E-Gene,
AsA; TBC1D30, P-Gene, AsA; TBC1D9B, E-Gene, AsA; TBL2, E-Gene, AsA; TBX6, E-Gene, AsA;
TCEB1, P-Gene, AsA; TCF7, E-Gene, AsA; TCF7, P-Gene, AsA; TCF7, T-Gene, AsA; TCHP, E-
Gene, AsA; TCN2, E-Gene, AsA; TCP11, E-Gene, AsA; TCP11L1, P-Gene, AsA; TCTEX1D4, E-
Gene, AsA; TDGF1, E-Gene, AsA; TDH, E-Gene, AsA; TDO2, P-Gene, AsA; TDRD5, E-Gene, AsA;
TDRD5, P-Gene, AsA; TDRD6, E-Gene, AsA; TDRD9, E-Gene, AsA; TEKT2, E-Gene, AsA; TERT,
P-Gene, AsA; TESPA1, E-Gene, AsA; TET3, T-Gene, AsA; TFAP2E, E-Gene, AsA; TFB2M, E-
Gene, AsA; TFB2M, P-Gene, AsA; TGFB2-AS1, E-Gene, AsA; THUMPD1, E-Gene, AsA; THY1, T-
Gene, AsA; TIAM1, P-Gene, AsA; TIGD4, P-Gene, AsA; TLR7, P-Gene, AsA; TM4SF20, E-Gene,
AsA; TM9SF2, E-Gene, AsA; TM9SF2, P-Gene, AsA; TMED6, E-Gene, AsA; TMEFF2, P-Gene,
AsA; TMEM106C, E-Gene, AsA; TMEM120A, E-Gene, AsA; TMEM133, E-Gene, AsA; TMEM140,
E-Gene, AsA; TMEM141, E-Gene, AsA; TMEM144, E-Gene, AsA; TMEM151A, E-Gene, AsA;
TMEM18, T-Gene, AsA; TMEM181, E-Gene, AsA; TMEM181, P-Gene, AsA; TMEM2, P-Gene,
AsA; TMEM2, T-Gene, AsA; TMEM232, P-Gene, AsA; TMEM245, P-Gene, AsA; TMEM248, E-
Gene, AsA; TMEM25, T-Gene, AsA; TMEM253, E-Gene, AsA; TMEM35B, E-Gene, AsA;
TMEM50A, E-Gene, AsA; TMEM52, E-Gene, AsA; TMEM52, P-Gene, AsA; TMPRSS4, T-Gene,
AsA; TMPRSS7, E-Gene, AsA; TMTC2, P-Gene, AsA; TNF, P-Gene, AsA; TNF, T-Gene, AsA;
TNFAIP3, C-Gene, AsA; TNFAIP3, P-Gene, AsA; TNFAIP3, T-Gene, AsA; TNFAIP6, E-Gene,
AsA; TNFSF10, P-Gene, AsA; TNFSF4, E-Gene, AsA; TNFSF4, P-Gene, AsA; TNIP1, E-Gene,
AsA; TNIP1, P-Gene, AsA; TNKS, P-Gene, AsA; TNPO3, E-Gene, AsA; TNPO3, T-Gene, AsA;
TNR, P-Gene, AsA; TNRC18, E-Gene, AsA; TNXA, E-Gene, AsA; TNXB, P-Gene, AsA; TNXB, T-
Gene, AsA; TONSL, E-Gene, AsA; TP53I3, E-Gene, AsA; TP53INP1, E-Gene, AsA; TPCN2, E-
Gene, AsA; TPI1P2, T-Gene, AsA; TPRKB, T-Gene, AsA; TPST1I, E-Gene, AsA; TRAPPC4, T-
Gene, AsA; TRAV12-1, P-Gene, AsA; TRAV3, P-Gene, AsA; TRAV8-5, E-Gene, AsA; TRBC1, E-
Gene, AsA; TRBC2, E-Gene, AsA; TRBV19, E-Gene, AsA; TRBV21-1, E-Gene, AsA; TRBV24-1,
E-Gene, AsA; TRBV25-1, P-Gene, AsA; TRBV26OR9-2, E-Gene, AsA; TRBV27, E-Gene, AsA;
TRBV28, E-Gene, AsA; TRBV29-1, E-Gene, AsA; TRBV30, E-Gene, AsA; TRBVB, E-Gene, AsA;
TREH, T-Gene, AsA; TRIM22, E-Gene, AsA; TRIM39-RPP21, T-Gene, AsA; TRIM5, E-Gene, AsA;
TRIM5, P-Gene, AsA; TRIM6, E-Gene, AsA; TRMT13, P-Gene, AsA; TRNAI6, T-Gene, AsA;
TRPM1, E-Gene, AsA; TRPM3, T-Gene, AsA; TRPV2, E-Gene, AsA; TRPV4, E-Gene, AsA;
TRPV5, E-Gene, AsA; TRPV6, E-Gene, AsA; TSC22D1, E-Gene, AsA; TSC22D1, T-Gene, AsA;
TSLP, E-Gene, AsA; TSPAN33, T-Gene, AsA; TSPEAR-AS2, E-Gene, AsA; TTC23, E-Gene, AsA;
TTC23, P-Gene, AsA; TTC27, E-Gene, AsA; TTC28, E-Gene, AsA; TTC28, P-Gene, AsA; TTC36,
T-Gene, AsA; TTK, E-Gene, AsA; TTK, P-Gene, AsA; TTLL2, P-Gene, AsA; TTLL3, E-Gene, AsA;
TTLL4, E-Gene, AsA; TUB, E-Gene, AsA; TUB, P-Gene, AsA; TUBA3E, E-Gene, AsA; TUBA3E,
P-Gene, AsA; TUBAL3, E-Gene, AsA; TUBAL3, P-Gene, AsA; TUBB, T-Gene, AsA; TWIST2, E-
Gene, AsA; TXNDC5, E-Gene, AsA; TYW1, E-Gene, AsA; TYW3, E-Gene, AsA; U1, P-Gene, AsA;
U2.51, T-Gene, AsA; U2AF1L4, E-Gene, AsA; U3, P-Gene, AsA; U4, P-Gene, AsA; U4atac, P-Gene,
AsA; U6, P-Gene, AsA; U6.1196, T-Gene, AsA; U6.1230, T-Gene, AsA; U6.347, T-Gene, AsA;
U6.408, T-Gene, AsA; U6.414, T-Gene, AsA; U6.484, T-Gene, AsA; U6.546, T-Gene, AsA; U6.55,
T-Gene, AsA; U6.612, T-Gene, AsA; U6.898, T-Gene, AsA; U6atac, P-Gene, AsA; U7.110, T-Gene,
AsA; U7.74, T-Gene, AsA; U7.79, T-Gene, AsA; UBA52, E-Gene, AsA; UBAC2-AS1, E-Gene, AsA;
UBAP1, E-Gene, AsA; UBE2G1, P-Gene, AsA; UBE2R2, E-Gene, AsA; UBLCP1, E-Gene, AsA;
UBQLNL, E-Gene, AsA; UCP2, E-Gene, AsA; UEVLD, P-Gene, AsA; UGDH, E-Gene, AsA;
UGT2A1, P-Gene, AsA; UGT2B4, E-Gene, AsA; UGT3A2, P-Gene, AsA; UHRF1BP1, E-Gene,
AsA; UIMC1, P-Gene, AsA; ULK4P2, E-Gene, AsA; ULK4P3, E-Gene, AsA; UNC13C, P-Gene,
AsA; UOX, E-Gene, AsA; UPK1A, E-Gene, AsA; UPK1A-AS1, E-Gene, AsA; UPK2, T-Gene, AsA;
URI1, P-Gene, AsA; UROD, E-Gene, AsA; USH2A, P-Gene, AsA; USP25, P-Gene, AsA; USP3, E-
Gene, AsA; USP3, P-Gene, AsA; USP3-AS1, E-Gene, AsA; USP32P1, E-Gene, AsA; USP37, E-
Gene, AsA; USP42, P-Gene, AsA; USP47, E-Gene, AsA; USP47, P-Gene, AsA; UST, P-Gene, AsA;
UTP15, E-Gene, AsA; UTP4, E-Gene, AsA; UTP6, E-Gene, AsA; UTP6, P-Gene, AsA; VAC14, E-
Gene, AsA; VAC14, P-Gene, AsA; VAC14-AS1, E-Gene, AsA; VARS2, E-Gene, AsA; VDAC1, T-
Gene, AsA; VIL1, E-Gene, AsA; VKORC1L1, E-Gene, AsA; VN1R32P, E-Gene, AsA; VN1R81P, E-
Gene, AsA; VN1R84P, E-Gene, AsA; VPS28, E-Gene, AsA; VPS29, E-Gene, AsA; VPS37C, E-
Gene, AsA; VPS53, E-Gene, AsA; VPS8, P-Gene, AsA; VRK2, E-Gene, AsA; VRK2, P-Gene, AsA;
WASF5P, E-Gene, AsA; WASHC3, E-Gene, AsA; WBSCR17, P-Gene, AsA; WDFY4, C-Gene, AsA;
WDFY4, E-Gene, AsA; WDFY4, P-Gene, AsA; WDR36, E-Gene, AsA; WDR36, P-Gene, AsA;
WDR69, P-Gene, AsA; WDR91, E-Gene, AsA; WDR92, P-Gene, AsA; WFDC13, P-Gene, AsA;
WIPF3, T-Gene, AsA; WNT3A, E-Gene, AsA; WNT3A, P-Gene, AsA; WNT9A, E-Gene, AsA;
WSCD1, P-Gene, AsA; WWC1, E-Gene, AsA; WWC1, P-Gene, AsA; WWOX, P-Gene, AsA; XCR1,
E-Gene, AsA; XCR1, P-Gene, AsA; XDH, E-Gene, AsA; XPR1, P-Gene, AsA; XRCC3, E-Gene,
AsA; XRRA1, E-Gene, AsA; XXbac-BPG154L12.4, P-Gene, AsA; XXbac-BPG154L12.4, T-Gene,
AsA; XXbac-BPG246D15.9, P-Gene, AsA; XXbac-BPG246D15.9, T-Gene, AsA; XXbac-
BPG248L24.13, P-Gene, AsA; XXbac-BPG249D20.9, T-Gene, AsA; XXbac-BPG25018.13, T-Gene,
AsA; XXbac-BPG254F23.7, P-Gene, AsA; XYLT1, P-Gene, AsA; Y_RNA, E-Gene, AsA; Y_RNA,
P-Gene, AsA; YEATS4, E-Gene, AsA; YIPF4, E-Gene, AsA; YPEL3, E-Gene, AsA; YWHAE, P-
Gene, AsA; YWHAH, E-Gene, AsA; YWHAQ, E-Gene, AsA; Z84485.1, E-Gene, AsA; Z93930.3, E-
Gene, AsA; ZBED3, E-Gene, AsA; ZBED3-AS1, E-Gene, AsA; ZBTB11, E-Gene, AsA; ZBTB11-
AS1, E-Gene, AsA; ZBTB20, P-Gene, AsA; ZBTB32, E-Gene, AsA; ZBTB8OS, P-Gene, AsA;
ZC3H10, E-Gene, AsA; ZC3H12C, P-Gene, AsA; ZCCHC11, T-Gene, AsA; ZDHHC20, E-Gene,
AsA; ZDHHC20, P-Gene, AsA; ZDHHC20-IT1, E-Gene, AsA; ZDHHC3, E-Gene, AsA; ZDHHC3,
P-Gene, AsA; ZDHHC4, E-Gene, AsA; ZFAND5, T-Gene, AsA; ZFHX2, T-Gene, AsA; ZFP57, P-
Gene, AsA; ZFP90, E-Gene, AsA; ZFP90, P-Gene, AsA; ZFPM1, P-Gene, AsA; ZIC2, P-Gene, AsA;
ZKSCAN3, P-Gene, AsA; ZKSCAN3, T-Gene, AsA; ZKSCAN4, T-Gene, AsA; ZMYM1, E-Gene,
AsA; ZMYM4, E-Gene, AsA; ZMYND15, E-Gene, AsA; ZNF100, E-Gene, AsA; ZNF100, P-Gene,
AsA; ZNF107, E-Gene, AsA; ZNF117, E-Gene, AsA; ZNF12, E-Gene, AsA; ZNF132, E-Gene, AsA;
ZNF138, P-Gene, AsA; ZNF142, E-Gene, AsA; ZNF165, P-Gene, AsA; ZNF165, T-Gene, AsA;
ZNF175, E-Gene, AsA; ZNF184, T-Gene, AsA; ZNF187, T-Gene, AsA; ZNF192, T-Gene, AsA;
ZNF208, E-Gene, AsA; ZNF227, P-Gene, AsA; ZNF24, E-Gene, AsA; ZNF251, E-Gene, AsA;
ZNF251, P-Gene, AsA; ZNF273, E-Gene, AsA; ZNF300P1, E-Gene, AsA; ZNF311, T-Gene, AsA;
ZNF316, E-Gene, AsA; ZNF337, E-Gene, AsA; ZNF365, E-Gene, AsA; ZNF365, P-Gene, AsA;
ZNF391, T-Gene, AsA; ZNF396, E-Gene, AsA; ZNF396, P-Gene, AsA; ZNF429, E-Gene, AsA;
ZNF430, E-Gene, AsA; ZNF430, P-Gene, AsA; ZNF431, E-Gene, AsA; ZNF438, E-Gene, AsA;
ZNF438, P-Gene, AsA; ZNF451, P-Gene, AsA; ZNF493, E-Gene, AsA; ZNF497, E-Gene, AsA;
ZNF497, P-Gene, AsA; ZNF517, E-Gene, AsA; ZNF525, E-Gene, AsA; ZNF584, E-Gene, AsA;
ZNF608, E-Gene, AsA; ZNF608, P-Gene, AsA; ZNF624, P-Gene, AsA; ZNF626, E-Gene, AsA;
ZNF641, E-Gene, AsA; ZNF66, E-Gene, AsA; ZNF680, E-Gene, AsA; ZNF708, E-Gene, AsA;
ZNF714, E-Gene, AsA; ZNF738, E-Gene, AsA; ZNF76, E-Gene, AsA; ZNF763, P-Gene, AsA;
ZNF765, P-Gene, AsA; ZNF771, P-Gene, AsA; ZNF815P, E-Gene, AsA; ZNF827, E-Gene, AsA;
ZNF827, P-Gene, AsA; ZNF837, E-Gene, AsA; ZNF85, E-Gene, AsA; ZNF890P, E-Gene, AsA;
ZNRD1, T-Gene, AsA; ZNRD1-AS1, P-Gene, AsA; ZNRD1-AS1, T-Gene, AsA; ZPBP, T-Gene,
AsA; ZSCAN12, T-Gene, AsA; ZSCAN16, P-Gene, AsA; ZSCAN16, T-Gene, AsA; ZSCAN23, T-
Gene, AsA; ZSCAN30, E-Gene, AsA; ZSWIM5, E-Gene, AsA; ZYG11A, E-Gene, AsA; ZYG11B, E-
Gene, AsA; ZYG11B, T-Gene, AsA;

TABLE 20
Cluster analysis of SLE SNP-predicted protein clusters
using genes derived from the AsA validation GWAS SNP cohort.
Gene set enrichments for each cluster were determinged using
BIG-C (functional categories), I-SCOPE (cellular catgories)
and IPA (canonical pathways). Functional categories in
bold-face indicate those the lowest P-value and highest
odds ratio. P-values are from Fisher's exact test that
measures the significance of overlap between analysis-ready
genes in each cluster and genes within an annotation.
AsA GWAS pathways and functional/cellular enrichment

| | Functional | Cellular | | |
|---|---|---|---|---|
| Cluster | categories | categories | IPA Canonical Pathway | P value |
| 9 | PRRs | | Kinetochore metaphase signaling | 1.36E−09 |
| | | | pathway | |
| | IFN-stimulated | | HIPPO signaling | 8.54E−06 |
| | genes | | | |
| | Pro-cell cycle | | Cell cycle control of chromosomal | 3.35E−05 |
| | | | replication | |
| | Intracellular | | ATM signaling | 2.85E−04 |
| | signaling | | | |
| | | | Protein kinase A signaling | 3.19E−04 |
| 25 | Immune | B cells | Neurinflammation signaling pathway | 1.35E−03 |
| | signaling | | | |
| | | pDCs | ErbB signaling | 2.21E−03 |
| | | | Neuregulin signaling | 2.75E−03 |
| | | | IL-15 production | 3.63E−03 |
| | | | PI3K singnaling in B lymphocytes | 4.69E−03 |
| 2 | Immune cell | T & B cells | CREB singaling in neurons | 5.83E−18 |
| | surface | | | |
| | General cell | | Breast cancer regulation by strathmin 1 | 1.61E−16 |
| | surface | | | |
| | | | cAMP mediated signaling | 1.71E−12 |
| | | | | 1.02E−11 |
| | | | G protein coupled receptor signaling | |
| | | | G alpha i signaling | 9.39E−10 |
| 1 | Ub & | | EIF2 signaling | 9.26E−14 |
| | sumoylation | | | |
| | mRNA splicing | | Regulation of eIF4 and p70S6K signaling | 3.09E−08 |
| | | | mTOR singaling | 1.90E−07 |
| | | | Coronavirus pathogenesis pathway | 2.96E−07 |
| | | | Protein ubiquitination pathway | 1.57E−05 |
| 3 | mRNA | | Sytemic lupus erythematosus signaling | 6.55E−06 |
| | processing | | | |
| | mRNA splicing | | Spliceosomal cycle | 1.26E−04 |
| | | | Assembly of RNA polymerase II complex | 1.42E−04 |
| | | | Cleavage and polyedenylation of pre- | 2.61E−04 |
| | | | mRNA | |
| | | | Nuclear excision repair pathway | 2.29E−03 |
| 5 | Endocytosis | | Cholecystokon/gastrin mediated signaling | 4.27E−07 |
| | Immune | | CREB signaling in neurons | 1.46E−06 |
| | secreted | | | |
| | | | Cardiac hypertrophy signaling | 2.72E−06 |
| | | | (enhanced) | |
| | | | GNRH signaling | 3.79E−06 |
| | | | Endothelin-1 signaling | 6.12E−06 |
| 6 | Proteasome | | Antigen presentation pathway | 1.96E−07 |
| | MHC class I | | Protein ubiquitination pathway | 1.32E−06 |
| | Pro-cell cycle | | Interferon signaling | 1.44E−05 |
| | | | Primary immunodecificiency signaling | 1.98E−03 |
| | | | Prostate cancer signaling | 6.41E−03 |
| 4 | Pro-cell cycle | | HIPPO signaling | 1.40E−03 |
| | Intracellular | | 14-3-3 mediated signaling | 3.10E−03 |
| | signaling | | | |
| | | | Coronavirus pathogenesis pathway | 2.93E−02 |
| | | | Cell cycle: G2/M DNA damage checkpoint | 3.19E−02 |
| | | | regulation | |
| | | | remodeling of epithelial adherens | 4.40E−02 |
| | | | junctions | |
| 22 | mRNA | | no enrichment | |
| | processing | | | |
| 17 | Transcrption | | no enrichment | |
| | factors | | | |
| 23 | Autophagy | | D-myo-inositol (1,4,5)-trisphosphate | 7.94E−11 |
| | | | biosynthesis | |
| | | | Superpathway of inositol phosphate | 2.68E−09 |
| | | | compounds | |
| | | | D-myo-inositol-5-phosphate metabolism | 1.77E−05 |
| | | | Aldersterone signaling in epithelial cells | 1.80E−05 |
| | | | 3-phosphoinositide biosynthesis | 2.09E−09 |
| 32 | Autophagy | | Tetrahydrolfolate salvage from 5,10- | 1.32E−03 |
| | | | methenyltetrahydrofolate | |
| | | | Histidine degradation III | 2.11E−03 |
| | | | Folate transformations I | 2.37E−03 |
| | | | GABA receptor signaling | 2.48E−02 |
| 13 | microRNA | | Inhibition of ARE-mediated mRNA | 5.26E−05 |
| | processing | | degradation pathway | |
| | | | HOTAIR regulatory pathway | 1.18E−04 |
| | | | Regulation of eIF4 and p70S6K signaling | 1.31E−04 |
| | | | EIF2 signaling | 3.17E−04 |
| | | | Insulin secretion signaling pathway | 4.08E−04 |
| 24 | Lysosome | | Iron homeostasis pathway | 3.41E−05 |
| | | | Phagosome maturation | 4.56E−05 |
| | | | Oxidative phosphorylation | 1.22E−03 |
| | | | Mitochondrial dysfunction | 2.96E−03 |
| 18 | ROS protection | | Choindroitin sulfate degradation | 7.89E−05 |
| | | | Glutathione redox reactions I | 1.81E−04 |
| | | | Glutathione mediated detoxification | 3.24E−04 |
| | | | Glutamate removal from folates | 8.36E−04 |
| | | | UDP-D-xylose and UDP-D-glucuronate | 1.67E−03 |
| | | | biosynthesis | |
| 28 | Glycolysis | | Fatty acid alpha oxidation | 1.03E−09 |
| | | | Eicosanoid signaling | 1.50E−07 |
| | | | Histamine degradation | 2.35E−07 |
| | | | Oxidative ethanol degradation III | 3.34E−07 |
| | | | Putrescine degradation III | 4.58E−07 |
| 21 | Fatty acid | | iNOS signaling | 5.80E−06 |
| | biosynthesis | | | |
| | Peroxisomes | | Citrulline nitric oxide cycle | 5.92E−06 |
| | | | Necroptosis signaling pathway | 6.22E−06 |
| | | | NF-KB signaling | 1.04E−05 |
| | | | Dendritic cell maturation | 1.16E−05 |
| 7 | Mito. TCA cycle | | DNA methylation and transcriptional | 1.89E−04 |
| | | | repression signaling | |
| | | | Transcriptional regulatory network in ES | 4.52E−04 |
| | | | cells | |
| | | | NER pathway | 1.63E−03 |
| | | | 2-ketoglutartate dehydrogenase complex | 2.35E−03 |
| | | | Branched-chain a-keto acid | 2.35E−03 |
| | | | dehydrogenase complex | |
| 8 | Integrin | Monocytes | Caveolar-mediated endocytosis signaling | 1.25E−07 |
| | signaling | | | |
| | Secreted & ECM | | Regulation of cellular mechanics by | 6.15E−06 |
| | | | calpain proteases | |
| | | | Actin-nucleation by ARP-WASP complex | 8.30E−06 |
| | | | | 2.34E−05 |
| | | | Regulation of Actin-based motility by Rho | |
| | | | FAK signaling | 2.53E−05 |
| 12 | Integrin | | GP6 signaling [athway | 2.77E−06 |
| | signaling | | | |
| | | | Hepatic fibrosis | 1.06E−05 |
| | | | Apelin liver signaling pathway | 6.84E−03 |
| | | | Intrinsic prothrombin activation pathway | 1.10E−02 |
| | | | Activation of IRF by cytosolic PRRs | 1.65E−02 |
| 33 | General cell | | G alpha 12/13 signaling | 1.00E−13 |
| | surface | | | |
| | | | Signaling by Rho faily GTPases | 2.88E−09 |
| | | | Synaptogenesis signaling pathway | 1.01E−08 |
| | | | RhoGDi signaling | 4.60E−08 |
| | | | Germ cell-sertoli cell junction signaling | 1.13E−04 |
| 14 | | | Synaptogenesis signaling pathway | 3.90E−04 |
| | | | Sperm motility | 4.97E−03 |
| | | | tRNA splicing | 2.06E−02 |
| | | | nNOS signaling in neurons | 2.25E−02 |
| | | | Glutamate receptor signaling | 2.27E−02 |
| 34 | Endosome & | | Role of MAPK signaling in the | 3.12E−02 |
| | vesicle | | pathogenesis of influenza | |

TABLE 21
Cluster analysis of differentially expressed genes
from the whole blood of AsA SLE patients. Gene set
enrichments for each cluster were determinged using
BIG-C (functional categories), I-SCOPE (cellular catgories)
and IPA (canonical pathways). Functional categories in
bold-face indicate those the lowest P-value and highest
odds ratio. P-values are from Fisher's exact test that
measures the significance of overlap between analysis-ready
genes in each cluster and genes within an annotation.
DE pathways and functional/cellular enrichment

| | Functional | Cellular | | |
|---|---|---|---|---|
| Cluster | categories | categories | IPA Canonical Pathway | P value |
| 5 | IFN-stimulated | | Antigen presentation pathway | 3.75E−21 |
| | genes | | | |
| | MHC class I | | PD-1, PD-1L cancer immunotherapy | 3.50E−14 |
| | | | pathway | |
| | MHC class II | | B cell development | 5.71E−12 |
| | PRRs | | IL-4 signaling | 2.96E−11 |
| | | | Phagosome maturation | 7.69E−11 |
| 3 | Immune cell | T & myeloid | Complement system | 1.43E−03 |
| | surface | | | |
| | | | Phagosome formation | 1.47E−03 |
| | | | ERK/MAPK signaling | 4.09E−03 |
| | | | Glioma invasiveness signaling | 5.45E−03 |
| | | | Regulation of cellular mechanics by | 8.00E−03 |
| | | | calpain protease | |
| 29 | PRRs | B & myeloid | RANK signaling in osteoclasts | 6.10E−14 |
| | Pro-apoptosis | | CD27 signaling in lymphocytes | 2.36E−13 |
| | | | B cell receptor signaling | 2.87E−09 |
| | | | TNFR1 signaling | 2.97E−09 |
| | | | NGF signaling | 3.25E−09 |
| 18 | Immune cell | LDGs | SPINK1 pancreatic cancer pathway | 2.39E−03 |
| | surface | | | |
| | | Monocytes | Systemic lupus erythematosus in B cell | 4.44E−03 |
| | | | signaling pathway | |
| | | T, B & | Role of NFAT in regulation of the | 4.56E−03 |
| | | myeloid | immune response | |
| | | Granulocytes | Breast cancer regulation by strathmin 1 | 5.31E−03 |
| | | | CREB signaling in neurons | 5.63E−03 |
| 2 | Ub & | | Protein ubiquitination pathway | 5.01E−16 |
| | sumoylation | | | |
| | Unfolded protein | | Hypoxia signaling in the cardiovascular | 3.71E−13 |
| | & stress | | system | |
| | | | Adipogenesis pathway | 5.69E−03 |
| | | | Mitotic roles of polo like kinases | 1.35E−03 |
| | | | Kinetochore metaphase signaling | 3.23E−03 |
| | | | pathway | |
| 1 | mRNA splicing | | EIF2 signaling | 5.59E−30 |
| | mRNA processing | | Regulation of eIF4 and p70S6K | 6.30E−16 |
| | | | signaling | |
| | Transcription | | Spliceosomal cycle | 4.26E−14 |
| | factors | | | |
| | | | mTOR singaling | 2.96E−12 |
| | | | Coronavirus pathogenesis pathway | 7.04E−05 |
| 6 | mRNA splicing | | UVC-induced MAPK signaling | 3.06E−05 |
| | mRNA processing | | Kinetochore metaphase signaling | 3.39E−05 |
| | | | pathway | |
| | Transcription | | Telomerase signaling | 3.71E−05 |
| | factors | | | |
| | Nucleus & | | Mitotic roles of polo like kinases | 8.47E−05 |
| | nucleolus | | | |
| | | | IL-8 signaling | 9.77E−05 |
| 7 | Chromatin | T cells | VDR/RXR activation | 2.67E−08 |
| | remodeling | | | |
| | IFN-stimulated | | Adipogenesis pathway | 1.88E−06 |
| | genes | | | |
| | Immune signaling | | PPARa/RXRa activation | 3.09E−06 |
| | | | TR/RXR activation | 1.31E−05 |
| | | | IL-6 signaling | 1.43E−05 |
| 33 | mRNA | | Adenine and adenosine salvage I | 2.46E−03 |
| | processing | | | |
| | | | Methionine degradation I | 1.79E−02 |
| | | | Cysteine biosynthesis III | 1.95E−02 |
| | | | Superpathway of methionine | 2.99E−02 |
| | | | degradation | |
| 31 | Chromatin | | Role of MAPK signaling in the | 3.01E−09 |
| | remodeling | | pathogenesis of influenza | |
| | Lysosome | | Iron homeostasis signaling pathway | 8.95E−09 |
| | | | Phagosome maturation | 1.76E−08 |
| | | | Regulation of cellular mechanics by | 4.88E−02 |
| | | | calpain protease | |
| 9 | Pro-cell cycle | | Cell cycle: G1/S checkpoint regulation | 4.98E−09 |
| | | | Glioma signaling | 1.87E−07 |
| | | | Cyclins and cell cycle regulation | 7.56E−07 |
| | | | Small cell lung cancer signaling | 1.46E−06 |
| | | | Chronic myeloid leukemia signaling | 2.51E−06 |
| 10 | Pro-apoptosis | T & myeloid | Death receptor signaling | 2.60E−12 |
| | Anti-apoptosis | | Necroptosis signaling | 2.25E−10 |
| | Cytoplasm & | | Neuroinflammation signaling pathway | 3.86E−10 |
| | biochemistry | | | |
| | | | Erythropoietin signaling pathway | 1.58E−08 |
| | | | Endothelin 1 signaling | 2.85E−08 |
| 12 | microRNA | | Inhibition of ARE-mediated mRNA | 5.82E−07 |
| | processing | | degradation pathway | |
| | | | Cell cycle: G2/M DNA damage | 1.85E−05 |
| | | | checkpoint regulation | |
| | | | HOTAIR regulatory pathway | 2.25E−05 |
| | | | Senescence pathway | 3.46E−03 |
| 20 | microRNA | T & myeloid | Melatonin degradation II | 4.14E−03 |
| | processing | | | |
| | | | BAG2 signaling pathway | 1.47E−11 |
| | | | FAT10 signaling pathway | 2.94E−10 |
| | | | Inhibition of ARE-mediated mRNA | 7.75E−10 |
| | | | degradation pathway | |
| | | | Huntington's disease signaling | 2.18E−08 |
| 25 | microRNA | | Inhibition of ARE-mediated mRNA | 5.82E−07 |
| | processing | | degradation pathway | |
| | | | Oxidized GTP and dGTP detoxification | 4.14E−03 |
| | | | Cell cycle regulation by BTG family | 3.77E−02 |
| | | | proteins | |
| 15 | Unfolded protein | APCs | Phagosome maturation | 5.77E−04 |
| | & stress | | | |
| | | NK cells | Tight junction signaling | 8.01E−04 |
| | | | Autophagy | 1.34E−03 |
| 16 | OXPHOS | | Oxidative phosphorylation | 2.62E.13 |
| | Mito. DNA to RNA | | Mitochondrial dysfunction | 6.20E−12 |
| | | | Sirtuin signaling | 2.78E−09 |
| 23 | Glycolysis | T & B cells | Aryl hydrocarbon receptor signaling | 2.45E−07 |
| | Chromatin | T, B & | Inflammasome pathway | 2.60E−07 |
| | remodeling | myeloid | | |
| | PRRs | T cells | RAR activation | 1.67E−06 |
| | Transcription | | Prolactin signaling | 2.97E−06 |
| | factors | | | |
| | | | Molecular mechanisms of cancer | 2.18E−05 |
| 19 | Peroxisomes | | DNA double strand break by non- | 9.89E−08 |
| | | | homologus end joining | |
| | Fatty acid | | ATM signaling | 1.59E−05 |
| | biosynthesis | | | |
| | Chromatin | | Assembly of RNA polymerase complex II | 2.08E−05 |
| | remodeling | | | |
| | | | Role of CHK proteins in cell cycle | 3.51E−05 |
| | | | checkpoint control | |
| | | | DNA double strand break repair by | 9.18E−04 |
| | | | homologus recombination | |
| 38 | Glycolysis | | IL-7 signaling pathway | 3.58E−09 |
| | | | Hepatic fibrosis signaling pathway | 2.49E−08 |
| | | | PI3K/AKT signaling | 3.78E−08 |
| | | | Role of osteoblasts, osteoclasts and | 8.50E−08 |
| | | | chondrocytes in RA | |
| | | | AMK signaling | 1.44E−07 |
| 56 | Mito. TCA cycle | | TCA cycle II | 2.87E−05 |
| | | | Glutamate degradation I | 6.91E−04 |
| | | | Glutamate biosynthesis II | 6.91E−04 |
| | | | Glutamate degradation X | 6.91E−04 |
| | | | 5-aminoimidazole ribonucleotide | 1.03E−03 |
| | | | biosynthesis I | |
| 11 | Mito. DNA to | | Protein ubiquitination pathway | 7.19E−03 |
| | RNA | | | |
| 4 | | | Granulocyte adhesion and diapedesis | 5.14E−09 |
| | | | Agranulocyte adhesion and diapedesis | 2.23E−07 |
| | | | G alpha i signaling | 2.12E−05 |
| | | | Breast cancer regulation by strathmin 1 | 2.80E−05 |
| | | | Leukocyte extravasation signaling | 1.37E−04 |
| 21 | Endosome & | | Synaptogenesis signaling | 4.75E−02 |
| | vesicle | | | |

TABLE 22
GSVA signatures (gene symbols)
Gene cluster: Interferon signatures/IFNA2
ACSL1; ADAR; AGT; AIM2; AKAP2; APOBEC3B; APOBEC3G; APOL3; ATF3; ATF5; BAG1;
BARD1; BCL7B; BLVRA; BRCA1; BRCA2; BST2; BUB1; C2; CACNA1A; CAD; CAMK2A;
CASP1; CASP10; CASP5; CBR1; CBWD1; CCL13; CCL7; CCL8; CCNA1; CCND2; CD2AP;
CD38; CD4; CD69; CDC42EP1; CDK4; CDKN1A; CFB; CH25H; CHKA; CNTN6; COL3A1;
CTSL; CXCL10; CXCL11; CXCL9; CXCR2; CYP2J2; DAB2; DEFB1; DLL1; DSC2; DUSP5;
DUSP7; DYNLT1; DYSF; ECE1; EDN1; EIF2AK2; EIF2B1; EIF4ENIF1; ENPP2; EPB41; ETV4;
F8; FAF1; FAS; FGF1; FLNA; FOXO1; FTL; FUT4; GADD45B; GBAP1; GBP1; GBP2; GCH1;
GCNT1; GLB1; GLS; GMPR; GPR161; GUK1; HBG2; HCAR3; HIST2H2AA3; HLA-DOA; HLA-
DRB5; HS6ST1; HSP90AA1; IDO1; IFI16; IFI27; IFI35; IFI44; IFI44L; IFI6; IFIT1; IFIT5; IFITM1;
IFITM2; IFITM3; IFNG; IFRD1; IGL; IKBKG; IL15; IL15RA; IL1RN; IL6; INPPL1; IRF2; IRF7;
ISG15; ISG20; ITIH2; JAK2; JUP; KCNA3; KDELR2; KIF20B; KLF6; KPNB1; KRT8; LAG3;
LAMP3; LAP3; LEPR; LGALS2; LGALS3BP; LGALS9; LGMN; LMNB1; LMO2; LY6E;
MAP2K5; MCL1; MED1; MGLL; MMP16; MNDA; MRPS15; MSR1; MX1; MX2; MYD88;
NAMPT; NFE2L3; NKTR; NMI; NR3C1; NUB1; NUPR1; OAS1; OAS2; OAS3; OSBPL1A; PATJ;
PDGFB; PDGFRL; PGGT1B; PKD2; PLSCR1; PMAIP1; PML; PRKRA; PSMB9; PTCH1; RBCK1;
RET; RGS1; RGS6; RPS9; RTP4; SAT1; SCARB2; SERPING1; SIT1; SLAMF1; SOCS1; SP100;
SP110; SP140; SPIB; ST3GAL5; STAP1; STAT1; STAT2; STX11; SUPT3H; SYN2; TAF5L; TAP1;
TAP2; TARBP1; TCN2; TFDP2; TGM1; TLR3; TLR7; TNFRSF11A; TNFSF10; TNFSF6; TNK2;
TOR1B; TRA2B; TRD; TRIM21; TRIM22; TRIM26; TRIM34; TRIM38; UBA7; UBE2L6; UBE2S;
UBE3A; UNC93B1; USP18; VAMP5; WARS; WT1; XAF1; © 20;
Gene cluster: Interferon signatures/IFNA1
ACLY; ACSL1; ADAM19; ADAP2; ADAR; ADGRE2; ADM; AFF3; AGT; AIM2; AKAP10;
AKAP2; ALOX12; ALOX5; ANXA4; APOBEC3B; APOBEC3G; APOL3; ATF3; ATF5; ATM;
ATP13A1; B4GAT1; BAG1; BAK1; BARD1; BCL11A; BCL7B; BGN; BLNK; BLVRA; BLZF1;
BRCA1; BRCA2; BST2; BUB1; C3AR1; CACNA1A; CAD; CALD1; CAMK2A; CAPN2; CASP1;
CASP10; CASP5; CBR1; CBWD1; CCL13; CCL3L1; CCL4; CCL7; CCL8; CCNA1; CCND2;
CCR1; CCR5; CCRL2; CD163; CD164; CD2AP; CD38; CD4; CD59; CD69; CD72; CD86; CDK17;
CDKN1A; CENPA; CENPE; CFB; CFLAR; CH25H; CHI3L2; CHKA; CISH; CKB; CMAHP;
CNTN6; CNTRL; COL3A1; COX17; CSF2RB; CTSL; CXCL10; CXCL11; CXCL2; CXCL9;
CXCR2; CYBB; CYP19A1; CYP2J2; DAB2; DEFA1; DEFB1; DHFR; DLL1; DMXL1; DNMT1;
DRAP1; DSC2; DUSP5; DUSP7; DYNLT1; DYSF; E2F1; ECE1; EDN1; EGR1; EIF2AK2; EIF2B1;
EIF4ENIF1; ELF1; ELF4; ENPP2; EPB41; ETV4; ETV6; F8; FAF1; FAS; FBXW2; FCGR1A;
FCMR; FGF1; FLNA; FMR1; FOXO1; FPR2; FTL; FUT4; GADD45B; GBAP1; GBP1; GBP2;
GCH1; GCNT1; GLS; GMPR; GPI; GPR161; GUK1; HBG2; HCAR3; HHEX; HIST2H2AA3; HK2;
HLA-DOA; HS6ST1; HSP90AA1; HSPA1A; HSPA1L; IDO1; IFI16; IFI27; IFI35; IFI44; IFI6;
IFIT1; IFIT5; IFITM1; IFITM2; IFITM3; IFNG; IFRD1; IGL; IKBKE; IKBKG; IL15; IL15RA;
IL18BP; IL18R1; IL1RN; IL6; IL7; INPP5D; INPPL1; IRF1; IRF2; IRF4; IRF7; IRF9; ISG15; ISG20;
ITGAL; ITGAX; JAK2; JCHAIN; JUP; KCNA3; KCNMB1; KDELR2; KIF20B; KLF2; KLF6;
KLRB1; KPNB1; KRT8; LAG3; LAMP3; LANCL1; LAP3; LBR; LEPR; LGALS2; LGALS3BP;
LGALS9; LGMN; LILRA1; LINC00597; LMNB1; LMO2; LTA; LTB4R; LY6E; LYN; MAP2K5;
MAP3K8; MARCKS; MBNL; MCL1; MED1; MEF2A; MFHAS1; MGLL; MNDA; MRPS15;
MS4A7; MSR1; MX1; MX2; MYD88; NAMPT; NAPSA; NBN; NCF1; NCOA2; NEBL; NEK4;
NFE2L3; NKTR; NMI; NOTCH1; NR3C1; NR4A3; NUB1; NUPR1; OAS1; OAS2; OAS3; PATJ;
PAX5; PAX8; PDE4B; PDGFB; PDGFRL; PFKFB3; PFKP; PIM2; PKD2; PLEK; PLSCR1;
PMAIP1; PML; PMS2; PPP2R2A; PRKAG1; PRKRA; PRKX; PSMB8; PSMB9; PTCH1; PTGER2;
RALB; RASGRP1; RBBP6; RBCK1; RERE; RGS1; RGS6; RIN1; RIPK1; RIPK3; RIPOR2;
RNF114; RPS6KA5; RPS9; RRBP1; RTP4; SAT1; SCARB2; SDS; SELL; SERPIND1; SERPING1;
SFTPB; SIDT2; SIT1; SLAMF1; SMO; SNX2; SOCS1; SOS1; SP100; SP110; SP140; SPIB; SPTA1;
SPTLC2; SRRM2; SSB; ST3GAL5; STAP1; STAT1; STAT2; STOML2; STX11; SUPT3H; TANK;
TAP1; TAP2; TAPBP; TARBP1; TBX21; TCN2; TFDP2; TFF1; TGM1; THY1; TLR1; TLR3;
TLR7; TNFAIP2; TNFRSF11A; TNFSF10; TNFSF6; TNK2; TOR1B; TRA2B; TRD; TRG; TRIM21;
TRIM22; TRIM26; TRIM34; TRIM38; TSPAN15; TXK; UBA7; UBE2L6; UBE2S; UBE3A;
UBQLN2; UNC93B1; USP15; USP18; USP25; USPL1; UVRAG; VAMP5; WARS; WIPF1; WT1;
XAF1; ZNF107;
Gene cluster: Interferon signatures/IFNG
ACLY; ACSL1; AFF2; AIM2; AKAP10; APOL3; ATF3; ATM; C1QB; C4A; CALD1; CASP1;
CASP10; CCL8; CCND2; CCR5; CD38; CDKN1A; CFB; CKB; CLEC10A; CPT1B; CSF2RB;
CTNND2; CXCL10; CXCL11; CXCL9; CYBB; EDN1; EPB41; ETAA1; ETV4; F8; FAS; FBLN1;
FBXL2; FCGR1A; FLII; GADD45B; GBP1; GBP2; GCH1; GCNT1; GLS; GSTM5; HBG2; HHEX;
HP; ICAM1; IDO1; IFI27; IFI44; IL15; IL15RA; IL18BP; IL1A; IL7; IRF1; IRF8; JAK2; JCHAIN;
KLF2; LAP3; LIMK2; LMNB1; MMP25; MRPS15; MSR1; NET1; NIN; NKTR; NLRP1; NR3C1;
OAS1; OAS3; P2RY13; PCDH9; PLA2G4C; PLEK; POLR2B; PSMB9; PTCH1; RALB; RGS1;
SERPIND1; SERPING1; SFTPB; SLAMF1; SLC1A5; SOCS1; SP100; SPRY4; SRRM2; STAT1;
STAT2; STX11; TAP1; TAP2; TBX21; TENM1; TFF1; TNFAIP2; TNFSF10; UBD; UBE2C;
UBE2L6; UBE3A; VAMP5; VSNL1; WARS; XRN1;
Gene cluster: Interferon signatures/IFNK
ACE2; ANKRD22; ANTXR2; APOBEC3F; B3GNT7; B4GALNT2; BBC3; BCL2L13; BEST3;
BMP4; BTN3A1; BTN3A3; C1orf141; C21orf91; C5orf56; CACNB4; CARD16; CASP7; CBR3;
CD274; CEMIP; CENPT; CLEC7A; DDX60L; FAM46C; FAM90A1; FBXO6; FER1L6; FRMPD1;
GBP5; GIMAP2; HCP5; HDX; HELZ2; HLA-B; HLA-F; HRASLS2; HSPG2; IFNK; IL22RA1;
KBTBD8; KCNB2; KIAA0040; KIAA1239; LHFPL2; LOC100130093; LOC100507463;
LOC153684; MAK; MALT1; MASTL; MBL1P; MMP13; NCOA7; NLRC5; OGFR; PARP14;
PLA2G4E; PLEKHA4; PPARGC1A; PPIF; PRDM8; PRKD2; PROX1; RASGRP3; RBM11;
RBM43; RHPN1-AS1; RNF122; RUNX2; SECTM1; SIDT1; SLC13A5; SLC15A3; SLC16A12;
SLC25A28; SLC25A36; SLC28A3; SLFN5; SP140L; STARD5; SYNPO2; TAGAP; TCF4;
TMEM140; TMEM229B; TMEM27; TNF; TRANK1; TREX1; TRIM25; TRIML2; TRPM6; UBA7;
WFDC5; ZFP42; ZHX2; ZNF107; ZNF608;
Gene cluster: Interferon signatures/IFNW1
ABCB10; ACLY; ACSL1; ADAR; ADM; AGT; AIM2; AKAP10; AKAP2; ALOX12; ANXA4;
APOBEC3B; APOBEC3G; APOL3; ATF3; ATF5; ATM; B4GAT1; BAG1; BARD1; BCL11A;
BCL7B; BLVRA; BLZF1; BRCA1; BRCA2; BRD4; BST2; C3AR1; CAD; CALD1; CAMK2A;
CAPN2; CASK; CASP1; CASP10; CASP5; CBR1; CBWD1; CCL13; CCL3L1; CCL7; CCL8;
CCNA1; CCND2; CCR1; CCR5; CCR7; CCRL2; CD164; CD2AP; CD38; CD4; CD47; CD59; CD69;
CDKN1A; CENPE; CFB; CFLAR; CHKA; CKB; CMAHP; CNTN6; CNTRL; COL3A1; CSF2RB;
CTSL; CXCL10; CXCL11; CXCL9; CXCR2; CYBB; CYP19A1; CYP2J2; DEFB1; DLL1; DSC2;
DUSP5; DUSP7; DYNLT1; DYSF; E2F1; ECE1; EDN1; EGR1; EIF2AK2; EIF2B1; EIF4ENIF1;
ENPP2; EPB41; ERCC4; ETV4; ETV6; F8; FAF1; FAS; FCER1G; FGF1; FGF13; FGL2; FLNA;
FMR1; FOXO1; FTL; FUT4; GADD45B; GBAP1; GBP1; GBP2; GCH1; GCNT1; GLB1; GLS;
GMPR; GPR161; GSTM5; GUK1; HBG2; HHEX; HIST2H2AA3; HLA-DOA; HS6ST1; HSP90AA1;
HSPA1A; IDO1; IFI16; IFI27; IFI35; IFI44; IFI6; IFIT1; IFIT5; IFITM1; IFITM2; IFITM3; IFRD1;
IGL; IKBKG; IL15; IL15RA; IL18R1; IL1RN; IL6; IL7; INPPL1; IRF1; IRF2; IRF7; IRF8; ISG15;
ISG20; ITIH2; JAK2; JCHAIN; JUP; KCNA3; KDELR2; KIF20B; KLF6; KPNB1; KRT8; LAG3;
LAMP3; LAP3; LEPR; LGALS2; LGALS3BP; LGALS9; LGMN; LINC00597; LMNB1; LMO2;
LY6E; LYN; MAP2K5; MARCKS; MBNL1; MCL1; MED1; MEF2A; MGLL; MLF1; MMP16;
MNDA; MRPS15; MS4A7; MSR1; MX1; MX2; MYD88; NAMPT; NCF1; NFE2L3; NKTR; NMI;
NPTX1; NR3C1; NUB1; NUPR1; OAS1; OAS2; OAS3; OSBPL1A; PATJ; PAX8; PDGFB;
PDGFRL; PKD2; PLEK; PLSCR1; PMAIP1; PML; PPP2R2A; PRKAG1; PRKRA; PSMB9; PTCH1;
PTGER2; RALB; RBBP6; RBCK1; RERE; RGS1; RGS6; RPS6KA5; RTP4; SAT1; SCARB2; SDS;
SELL; SERPIND1; SERPING1; SFT2D2; SIT1; SLC30A4; SOCS1; SOS1; SP100; SP110; SP140;
SPIB; SRRM2; ST3GAL5; STAP1; STAT1; STAT2; STX11; SUPT3H; TAP1; TAP2; TARBP1;
TBX21; TCN2; TFDP2; TFF1; TGM1; THY1; TLR3; TLR7; TNFAIP3; TNFRSF11A; TNFSF10;
TNFSF6; TNK2; TOR1B; TRA2B; TRD; TRIM21; TRIM22; TRIM34; TRIM38; UBA7; UBE2C;
UBE2L6; UBE2S; UNC93B1; USP18; USP25; WARS; WIPF1; WT1; XAF1; ZNF107;
Gene cluster: Interferon signatures/TYPE I and TYPE II IFN Core
ACSL1; AIM2; APOL3; ATF3; CASP1; CASP10; CCL8; CCND2; CD38; CDKN1A; CFB; CXCL10;
CXCL11; EDN1; EPB41; ETV4; F8; GADD45B; GBP1; GBP2; GCH1; GCNT1; GLS; HBG2; IDO1;
IFI27; IFI44; IL15; IL15RA; JAK2; LAP3; LMNB1; CXCL9; MRPS15; MSR1; NKTR; NR3C1;
OAS1; OAS3; PSMB9; PTCH1; RGS1; SERPING1; SOCS1; SP100; STAT1; STAT2; STX11;
TAP1; TAP2; FAS; TNFSF10; UBE2L6; WARS;
Gene cluster: Interferon signatures/TYPE I IFN Core
ACSL1; ADAR; AGT; AIM2; AKAP2; APOBEC3B; APOBEC3G; APOL3; ATF3; ATF5; BAG1;
BARD1; BCL7B; BLVRA; BRCA1; BRCA2; BST2; CAD; CAMK2A; CASP1; CASP10; CASP5;
CBR1; CBWD1; CCL13; CCL7; CCL8; CCNA1; CCND2; CD2AP; CD38; CD4; CD69; CDKN1A;
CFB; CHKA; CNTN6; COL3A1; CTSL; CXCL10; CXCL11; CXCL9; CXCR2; CYP2J2; DEFB1;
DLL1; DSC2; DUSP5; DUSP7; DYNLT1; DYSF; ECE1; EDN1; EIF2AK2; EIF2B1; EIF4ENIF1;
ENPP2; EPB41; ETV4; F8; FAF1; FAS; FASLG; FGF1; FLNA; FOXO1; FTL; FUT4; GADD45B;
GBAP1; GBP1; GBP2; GCH1; GCNT1; GLS; GMPR; GPR161; GUK1; HBG2; HIST2H2AA3;
HLA-DOA; HS6ST1; HSP90AA1; IDO1; IFI16; IFI27; IFI35; IFI44; IFI6; IFIT1; IFIT5; IFITM1;
IFITM2; IFITM3; IFRD1; IGL; IKBKG; IL15; IL15RA; IL1RN; IL6; INPPL1; IRF2; IRF7; ISG15;
ISG20; JAK2; JUP; KCNA3; KDELR2; KIF20B; KLF6; KPNB1; KRT8; LAG3; LAMP3; LAP3;
LEPR; LGALS2; LGALS3BP; LGALS9; LGMN; LMNB1; LMO2; LY6E; MAP2K5; MCL1; MED1;
MGLL; MNDA; MRPS15; MSR1; MX1; MX2; MYD88; NAMPT; NFE2L3; NKTR; NMI; NR3C1;
NUB1; NUPR1; OAS1; OAS2; OAS3; PATJ; PDGFB; PDGFRL; PKD2; PLSCR1; PMAIP1; PML;
PRKRA; PSMB9; PTCH1; RBCK1; RGS1; RGS6; RTP4; SAT1; SCARB2; SERPING1; SIT1;
SOCS1; SP100; SP110; SP140; SPIB; ST3GAL5; STAP1; STAT1; STAT2; STX11; SUPT3H; TAP1;
TAP2; TARBP1; TCN2; TFDP2; TGM1; TLR3; TLR7; TNFRSF11A; TNFSF10; TNK2; TOR1B;
TRA2B; TRD; TRIM21; TRIM22; TRIM34; TRIM38; UBA7; UBE2L6; UBE2S; UNC93B1; USP18;
WARS; WT1; XAF1;
Gene cluster: Interferon signatures/RIG-I Pathway
DDX58; IFIH1; DHX58; FADD; RIPK1; TRAF6; TRAF3; TRIM25; MAVS; RNF125; RNF122;
TNFAIP2; DHX29;
Gene cluster: Interferon signatures/DNA/RNA sensors
DHX36; DHX9; LRRFIP1; CGAS; IFI16; IFIX; ZBP1; MRE11; DDX41; STING1; TBK1; IRF3;
IFIH1; IFI16;
Gene cluster: Interferon signatures/TFN
ACLY; ACSL1; ADGRE2; AK3; AKAP10; AMPD3; APOL3; ARID3A; ARSE; ASAP1; B4GALT5;
BCL2A1; BHLHE41; BHMT; BIRC3; BRCA1; CALD1; CASP1; CASP10; CCL15; CCL20; CCL23;
CCL3L1; CD37; CD38; CD83; CDKN3; CKB; CR2; CTNND2; CXCL1; CXCL2; CXCL3; CXCL8;
CYP27B1; DAB2; EBI3; EGR1; EGR2; EPB41; EREG; ETAA1; F3; FABP1; FBXL2; FCER2;
FCGR2A; FLJ11129; FLNA; G0S2; GBP1; GCH1; GJB2; GLS; GMIP; GP1BA; GRK3; HCAR3;
HHEX; HOMER2; HP; ICAM1; IDO1; IFI44; IKBKG; IL16; IL18; IL1A; IL1B; IL1RN; IL6;
INHBA; INSIG1; ITGA6; KITLG; KLF1; KMO; LGALS3BP; MAP3K4; MARCKS; MGLL;
MMP19; MN1; MRPS15; MSC; MTF1; MX1; NAMPT; NELL2; NFKB1; NFKB2; NFKBIA;
NFKBIZ; NKX3-2; NR3C1; OAS3; PATJ; PDE4DIP; PDPN; PIAS4; PLAUR; PTGES; PTGS2;
RELB; RPGR; RPS9; SDC4; SERPIND1; SFRP1; SH3BP5; SLAMF1; SLC30A4; SOD2; SPI1;
SSPN; STAT4; TAF15; TAP2; TBX3; TFF1; TNF; TNFAIP2; TNFAIP3; TNFRSF11A; TRAF1;
TSC22D1; TYROBP; UBE2C; VEGFA; WT1;
Gene cluster: Metabolic & Oxidative stress signatures/Complement
C1QA; C1QB; C1QC; C2; C3; C4BPA; C4BPB; C5; C7; C6; C8a; C9; CFP;
Gene cluster: Metabolic & Oxidative stress signatures/OXPHOS
COX6B1; COX17; NDUFA5; COX6A2; NDUFA9; NDUFAB1; NDUFAF2; UQCR10; UQCRQ;
Gene cluster: Metabolic & Oxidative stress signatures/TCA cycle
ACO2; CS; DLAT; DLD; DLST; FH; GLUD1; IDH1; IDH2; IDH3A; IDH3B; IDH3G; MDH2;
MPC1; MPC2; OGDH; OGDHL; PDHA1; PDHA2; PDHB; PDHX; PDK1; PDK2; PDK3; PDK4;
PDP1; PDP2; PDPR; SDHA; SDHAF1; SDHAF2; SDHAF3; SDHAF4; SDHB; SDHC; SDHD;
SUCLA2; SUCLG1; SUCLG2; SUGCT;
Gene cluster: Metabolic & Oxidative stress signatures/Glycolysis
AKR1A; ALDH1A1; ALDH2; ALDOB; ENO1; FBP1; G6PD; GALE; GAPDH; GOT1; HKDC1;
LDHB; PCK1; PFKFB2; PFKP; PGM1; PKLR; RGN; SLC2A1; SLC2A5; SLC37A4;
Gene cluster: Metabolic & Oxidative stress signatures/Fatty Acid Beta Oxidation(FABO)
ABCD1; ABCD2; ABCD3; ACAA2; ACACB; ACAD11; ACADL; ACADM; ACADS; ACADVL;
ACAT1; ACAT2; ACOX1; ACOX2; ACOX3; ACOXL; ACSBG2; ACSL5; ADIPOQ; AKT2; AUH;
BDH2; CPT1A; CPT2; CROT; DECR1; ECHDC1; ECHDC2; ECHS1; ECI1; ECI2; EHHADH;
ETFA; ETFB; ETFDH; FABP1; GCDH; HADH; HADHA; HADHB; HIBCH; HSD17B4; IRS1;
IRS2; IVD; LEP; PEX2; PEX5; PEX7; SESN2; SLC25A17; SLC27A2; TWIST1;
Gene cluster: Metabolic & Oxidative stress signatures/Oxidative Stress
FOS; GPX1; GPX3; DUSP1; GPX4; PRDX1; PRDX3; PRDX4; PRDX5; S100A9;
Gene cluster: Metabolic & Oxidative stress signatures/Mitochondrial Dysfunction
COX17; COX6A2; COX6B1; NDUFA5; NDUFA9; NDUFAB1; NDUFAF2; UQCR10; UQCRQ;
ATP5MG;
Gene cluster: Shared signatures/TLR
CD14; CHUK; ECSIT; EIF2AK2; ELK1; FOS; IKBKB; IKBKG; IRAK1; JUN; LY96; MAP2K3;
MAP2K4; MAP2K6; MAP3K1; MAP3K14; MAP3K7; MAPK14; MAPK8; MYD88; NFKB1;
NFKBIA; PGLYRP1; PPARA; RELA; TAB1; TAB2; TIRAP; TLR10; TLR2; TLR3; TLR4; TLR6;
TLR7; TLR9; TOLLIP; TRAF6;
Gene cluster: Shared signatures/Cell cycle
ASPM; AURKA; AURKB; BRCA1; CCNB1; CCNB2; CCNE1; CDC20; CENPM; CEP55; E2F3;
GINS2; MCM10; MCM2; MKI67; NCAPG; NDC80; PTTG1; TYMS;
Gene cluster: Shared signatures/Inflammasome
AIM2; CASP1; CTSB; NLRC4; NLRP2; NOD2; PYCARD; P2RX7; NLRP1; RIPK2; POP1;
Gene cluster: Cell type signatures/Monocytes
BST1; C1QA; C1QB; C1QC; C1R; C1RL; CCL2; CCL8; CD14; CD163; CD300C; CD33; CD68;
CLEC12A; CLEC4D; CLEC4E; CSF2; CXCL1; CXCL2; CXCR2; FCGR1A; FCGR1B; FCGR3B;
FUT4; GRN; IK; IL18RAP; IL1B; IL1RN; LILRA5; LILRA6; LILRB2; LILRB3; MNDA; MRC1;
OSCAR; S100A8; SEMA4A; SIGLEC1; THBD;
Gene cluster: Cell type signatures/LDGs
AZU1; CAMP; CEACAM6; CEACAM8; CTSG; DEFA4; ELANE; LCN2; LTF; MPO; OLFM4;
RNASE3;
Gene cluster: Cell type signatures/B cells
BANK1; BLK; BLNK; CD22; CD79A; CD79B; DAPP1; FCRL1; FCRL2; FCRL3; FCRLA; GON4L;
GPR183; IGHM; KLHL6; MS4A1; PAX5; PLCL2; SH3BP5; SIGLEC6; VPREB1; ZNF318;
Gene cluster: Cell type signatures/T cells
CCR3; CD226; CD247; CD28; CD3D; CD3E; CD3G; CD4; CD5; CD8A; CD8B; ETS1; GATA3;
GRAP2; LEF1; SH2D1A; TRAC; TRBC1; TRDC;
Gene cluster: Cell type signatures/Anergic or activated T cells
CD160; CD244; CD96; CTLA4; KLRG1; LAG3; PBX3; TIGIT; VSIR;
Gene cluster: Cell type signatures/NK cells
KLRF1; NCAM1; NCR1; NCR3; SH2D1B;
Gene cluster: Cell type signatures/pDCs
CLEC4C; IL3RA; NRP1;
Gene cluster: Cell type signatures/Random
SH3YL1; TARP; VPS51; ARL2BP; RPS28; NSA2; ACYP2; SPOUT1; HIVEP2; SDR39U1; BRIX1;
FBXO21; PEBP1; PAOX; JADE1; POGLUT1; DENND2D; TMEM8B; EXOSC1; TMEM14B;
FAM159A; SLC2A4RG; DDX1; PHF5A; VKORC1; PSMC5; MAEA; KDSR; FKBP4; LINC01278;
SECISBP2L; ALKBH2; UBE2N; SLC3A2; CEP41; LYPLAL1; OXCT1; RANGAP1; SRSF4; NENF;
VDAC3; SLC30A5; ZNF275; PHF10; ACD; HSCB; ZNF485; PPP1R11; MCM7; RPUSD1; ZNF3;
AUH; ANAPC15; TNPO2; FAM192A; SCLT1; PI4KB; CALML4; C4orf32; CCNA2; SAP30L;
MANBAL; FAM45A; ATP11B; RBM10; SPTBN4; SPAG9; PTTG1IP; PRELID1; GGA3; ZNF493;
TAZ; RAP2C; RAB32; GPR1; RNU4-2; LRWD1; LARP4B; LILRB3; MYADM; ACTA2; CTRL;
TMEM170B; ABCA7; TMEM120A; PKM; NAMPT; MSL1; ACSL4; ZDHHC19; PELI1;
FAM214B; SLC2A3; TRIB1; COLGALT1; STAT3; MPO; OLR1; PSMD1; MAP3K3;

TABLE 23
SLE Datasets

| | | | | | SLE | Healthy | |
|---|---|---|---|---|---|---|---|
| Dataset | Sample Type | Sex | Ancestry | SLEDAI | Patients | Controls | |
| FDAPBMC3 | PBMC | Female | EA | 0-8 | 12 | 5 | # |
| GSE81622 | PBMC | Mixed | AsA | Unknown* | 12 | 21 | X |
| EMTAB11191 | Wholeblood | Mixed | AsA | Active >6 | 16 | 86 | X |
| GSE164457 | CD14+ monocytes | Mixed | EA and | 0-16 | 56 EA; | 0 | X |
| | (FACS sorted) | | AsA | | 61 AsA | | |

While preferred embodiments have been shown and described herein, it will be obvious to those skilled in the art that such embodiments are provided by way of example only. Numerous variations, changes, and substitutions will now occur to those skilled in the art without departing from the scope of the disclosure. It may be understood that various alternatives to the embodiments described herein may be employed in practice. Numerous different combinations of embodiments described herein are possible, and such combinations are considered part of the present disclosure. In addition, all features discussed in connection with any one embodiment herein may be readily adapted for use in other embodiments herein. It is intended that the following claims define the scope of the disclosure and that methods and structures within the scope of these claims and their equivalents be covered thereby.

1. A method for diagnosis of lupus in a patient, the method comprising:
a) analyzing a data set comprising or derived from gene expression measurements of at least 2 genes selected from the genes listed in each of one or more Tables selected from Tables: 1 to 11 to determine one or more sets of genes enriched in a biological sample obtained or derived from the patient; and
b) diagnosing lupus in the patient based on enrichment of the one or more sets of genes,
wherein the gene expression measurements are obtained from the biological sample.

2. The method of claim 1, wherein the data set comprises or is derived from gene expression measurements of effective number of genes selected from the genes listed in each of the one or more Tables selected from Tables: 1 to 11.

3. The method of claim 1, wherein the data set comprises or is derived from gene expression measurements of all genes listed in each of the one or more Tables selected from Tables: 1 to 11.

4. The method of claim 1, wherein Tables: 1 to 11 are selected.

5. The method of claim 1, wherein the data set is derived from the gene expression measurements using GSVA, gene set enrichment analysis (GSEA), enrichment algorithm, multiscale embedded gene co-expression network analysis (MEGENA), weighted gene co-expression network analysis (WGCNA), differential expression analysis, Z-score, log 2 expression analysis, or any combination thereof.

6. The method of claim 1, wherein the data set is derived from the gene expression measurements using GSVA.

7. The method of claim 6, wherein the data set comprises one or more GSVA scores of the patient, each GSVA score is generated based on one of the one or more selected Tables, wherein for each selected Table, the genes selected from the selected Table forms an input gene set for generating the GSVA score based on the selected Table, using GSVA.

8. The method of claim 1, further comprising administering a treatment to the patient based on the enrichment of the sets of genes.

9. The method of claim 8, wherein the treatment is configured to treat lupus.

10. The method of claim 8, wherein the treatment is configured to reduce severity of lupus.

11. The method of claim 8, wherein the treatment is configured to reduce risk of having lupus.

12. The method of claim 8, wherein: the one or more sets of genes comprise a set of genes selected from Table 1, and the treatment targets a JAK signaling pathway; the one or more sets of genes comprise a set of genes selected from Table 2, and the treatment targets an oxidative phosphorylation pathway; the one or more sets of genes comprise a set of genes selected from Table 3, and the treatment targets a sirtuin signaling pathway; the one or more sets of genes comprise a set of genes selected from Table 4, and the treatment targets a mitochondrial dysfunction pathway; the one or more sets of genes comprise a set of genes selected from Table 5, and the treatment targets a glycolysis pathway; the one or more sets of genes comprise a set of genes selected from Table 6, and the treatment targets a reactive oxygen species (ROS) protection pathway; the one or more sets of genes comprise a set of genes selected from Table 7, and the treatment targets an MTOR signaling pathway; the one or more sets of genes comprise a set of genes selected from Table 8, and the treatment targets a JAK signaling pathway; the one or more sets of genes comprise a set of genes selected from Table 9, and the treatment targets a microRNA processing pathway; the one or more sets of genes comprise a set of genes selected from Table 10, and the treatment targets a mitochondrial dysfunction pathway; the one or more sets of genes comprise a set of genes selected from Table 11, and the treatment targets a TNF signaling pathway; or any combination thereof.

13. The method of claim 12, wherein the treatment targeting the JAK signaling pathway comprises baricitinib, carfilzomib, curcumol, decernotinib, delgocitinib, ruxolitinib, solicitinib, tofacitinib, upadacitinib, bortezomib, densosumab, filgotinib, idelalisib, KZR-616, peficitinib, or any combination thereof; the treatment targeting the oxidative phosphorylation pathway comprises metformin, phenformin, BAY84-2243, CAI, ME344, fenofibrate, lonidamine, arsenic trioxide, atovaquone, hydrocortisone, a-TOS, thapsigargin, or any combination thereof; the treatment targeting the sirtuin signaling pathway comprises resveratrol, and/or cyclosporin A; the treatment targeting the mitochondrial dysfunction pathway comprises resveratrol, N-acetyl L-cysteine, SKQ1, ubiquinone, mito VitE, mitoTEMPO, vitamin E, vitamin C, or any combination thereof; the treatment targeting the glycolysis pathway comprises Cylcosporin A; the treatment targeting the reactive oxygen species (ROS) protection pathway comprises resveratrol, N-acetyl L-cysteine, SKQ1, ubiquinone, mitoVitE, mitoTEMPO, vitamin E, vitamin C, ALT-2074, Ebselen, GC4419, or any combination thereof; the treatment targeting the MTOR signaling pathway comprises sirolimus, everolimus, temsirolimus, or any combination thereof; the treatment targeting microRNA processing pathway comprises cyclosporin A, and/or thapsigargin; and the treatment targeting the TNF signaling pathway comprises adalimumab, AMG-811, baricitinib, BMS-986165, certolizumab, dacomitinib, etanercept, filgotinib, iguratimod, infliximab, ruxolitinib, solicitinib, tabalumab, trofinetide, upadacitinib, or any combination thereof.

14. The method of claim 1, wherein the biological sample comprises a blood sample, isolated peripheral blood mononuclear cells (PBMCs), a tissue biopsy sample, or any derivative thereof.

15. The method of claim 1, wherein the biological sample comprises a blood sample, isolated peripheral blood mononuclear cells (PBMCs), or any derivative thereof.

16. The method of claim 1, wherein the patient has lupus.

17. The method of claim 1, wherein the patient is at elevated risk of having lupus.

18. The method of claim 1, wherein the patient is suspected of having lupus.

19. The method of claim 1, wherein the patient is asymptomatic for lupus.

20. The method of claim 1, wherein the patient is of Asian ancestry and/or European ancestry.
